SEQUENCE LISTING (1626 sequences)

SEQ ID NO: 1 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 2 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 3 - skipped

SEQ ID NO: 4 - RNA, 69 nt, synthetic construct
  source 1..69: mol_type = other RNA
acagcttatttggaagctgaaatgtgaggtttataacactcacaagaatcctgaaatggtacatccaac

SEQ ID NO: 5 - skipped

SEQ ID NO: 6 - RNA, 20 nt, synthetic construct
  source 1..20: mol_type = other RNA
attgctccttacgaggagac

SEQ ID NO: 7 - protein, 36 aa, synthetic construct
SDLYIERGGDPRDVHQQVETKPKGKRKSEIRILKIR

SEQ ID NO: 8 - protein, 31 aa, synthetic construct
SDYIVDHGGDPEKVFFETKSKKDKTKRYKRR

SEQ ID NO: 9 - protein, 698 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 10 - protein, 704 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 11 - protein, 608 aa, synthetic construct
VNHYLRLTSLPALYAGWERVAANDGGPGVDRVSINKFDVALDRDLERLHRELVSFTYRCMPLRRYEIPKLNGKIRTLAVPTVRDRVVQSSALIVLQPVIERELERCSFAYRPGLSRLTAIEQIRQLREQGYRWVVDADIESFFDNVEFGLLLRRFRELVPENHTVALIEQWLKAPVLFHGRLIKRTKGLPQGSPISPVLANLFLDKFDEALLAHNHKLIRYADDFIILCKTKPRAEEALRLTEELLAGLHLRLNAEKTAVTSFDQGFKYLGAIFVKSIIIPQSGTKPQRRKKQMNAQSPDPPLRQNEKRRKSAAAKKKEEITVLAQALLEALNAKQMTTADLVRTPPTRPPVPNGAPEPPSAPPNVSPFMRTLYIQEQGCWLRLDGERFVVATGGEEGVALYEIPTVKVEQIMIFGTCLITPAAMRYALLHTIPITLLSSRGQYFGRIESTYGADITLERTQFLHSVDTAFVLQTARTIVQAKLRNTKALLQRHQTKHEMISAAIVQLTRLEDQVANATSVDQVRGYEGSGSAVFFDVFDELMPGKGFTFEKRTRRPPTDPVNAMLSFGYTLLFNNIYSMARLHRLNPYVGSLHAALSEHKQALTCSY

SEQ ID NO: 12 - protein, 362 aa, synthetic construct
MYRCVKLPVAENMPNTDSQLNTVGWDEIDWRKVERYVYKLHKRIYAASRRGDVKQVRKLQKTLMSSWSNQVLSVRKVTQDNRGKRTAGVDGIHILSSEARINLAGSLKITGKSHPTRRVWIPKPGTEEKRPLGIPTIYDRVVQNLVKTALEPEWEAIFEPNSFGFRPGRSCHDAIWQIKNSIQNRPKFILDADIAKCFDRINHLALLQKLGYTGKIRQQIKAWLKSGVIDRGAFTATSEGTMQGGVISPLLANIALHGMEKMLMEFAKTLDMRRNDKPNCQISWQQKVKSLTFVRYADDFVLIHKDLNVVRRCRDLISEWLKDIGLELKPSKTRIAHTKKIRERGSPEAKRYLYRGGNATRA

SEQ ID NO: 13 - protein, 579 aa, synthetic construct
MMAHRKIKANHGSKTKGMDKQNVIDFKNMVTEEYLSEIKLELLDYKSGLIRRKEIPKPNGGVRPLGISCYKDKIIEEAIKQVIEPIVEAKFHPDSYGFRPGRSAQEAIQMSQTLVRAGYTYACEFDIKKCFDNIKHRLIRKKLWALGIRDTRLLTIISKRLRANIYDPKTKTVSQSTLGTVQGGVLSPLLANVVLNSLDWWIASQWERFDFGIEENDYKNYASFHAAMNYRLNKTRLKSGRIVRYADDFVIFCKTEMEAKAWYFAVKDWMKCNLKLEISEEKSRIVDLTKDSIDFLGFELKANKEGKKCVKSVDRSNRGSYKYTKTSVTTKNLNKIIQTLKTKIKIILKTSDKKAKAVLITLLNITILGYHNYWRIASESSINFYNIYQRTKLSWWKLRRQADSKTHISPAFRKLYKEYVDSAFSIDGNTIYPIWGCRHTTLNPKKRDYNWYEDSPSIPENISSQMKIMCCNPIIHRSVEYNDNRLSKYSMQLGKDYITGKFVMAQDVHCHHKTMVSKGGKDNFDNLVILSKVTHGWVHQVNPEIPYMTKVQMDRLNDLREKCGREPLKNKKKSLKSEN

SEQ ID NO: 14 - protein, 430 aa, synthetic construct
MRKWYSLIDKVYARANLLEAFDKVRRNKGGKTSGIDGISVQAFKEHLTANLCQLHEELRSGTYRPQAVKRVYIDKEDGGKRPLGIPTVKDRVVQQALLNVLQPIFEPEFHPSSYGYRPGRSAHHAIAKAERFTRYYGLDHVVVMDLSKCFDTLDHELILSSVNEKVSDGKVLRLIDSFLKSGIIDKGKFEPTEKGSPQGGIISPLLTNIYLNKFDQYMKTLGIRIVRYADDILVFASTKSEAGKYRAIATTYLEGELMLTVNREKTHLTTPYKGIPYLGFEIHNYGVSASEKSIRKFKEKVRKLTPRNQGYKLDYYLTELNFLLKGYSMYFRVARSKSLFRNLMSWVRRRLRMMIMKSWKSWKGLHKQLRRMGYKGSLEKISVTRWRNSSCQLLHMALPNTWFDEQHLFNMEKVTTGTLHQYYNFVLNKV

SEQ ID NO: 15 - protein, 625 aa, synthetic construct
VNRAVLTLADLARPDALFDAWQQAERRKAGPGTDGQTVQVFGQQVEEHLEGLSKEIREGGYQPRPAQRIWMVKDSGGERGITIFTVRDRVALGAMRRTLGRLIGPTLSTASFAYREGLGALRAAHRLIEHRRSGRGWVVRSDIKEFFDTISHEILLEQLRVFLDARALELFGTILRTPIRDGYGISVPERGVAQGSPISPMLANLYLSGFDAALNCDGRGFVRFADDFVIAALSVKGAAEGLEIAQAQLEGLELRLNDDKTRIVSFEQGFDFLGFHFDADTVRVSETSLAEFKAHLEALLVSRFEQFSPGAVKKANDLIRGWRNYFQLGDVRKDYAALEEWIEARFGHKARQLERLLPDARGQRVPQLGGYGRVSRERQPRRSGQANTASARPQKRQRPYPNVKLDANDTPLTVRRNKAVLNVATLPALPETRVSRAILDELEQASLFHRANTACRWSTPDAADAVSNLAAAMSGRIPIRQAIEAYDKALFAQLKPGADPRGARGTLRANLRRIAWAALLEAGIDLGPEQPRKTPTLGNALADAYECLTADLALLAASRNDQLHAPQGAFERSLAFKPAYQRPGVQGQAHATAWMMILRLEAEAMRRMLETNGQTVYRVWRFGLP

SEQ ID NO: 16 - protein, 417 aa, synthetic construct
MEAVVERENMIAAYKRVVANRGAAGVDGMTVDALKAYLQPHWPRIRKELLEGRYRPQPVLEVEIPKPAGGMRKLGIPTVVDRLIQQAIHQVLEPIFDKDFSESSYGFRRGKSAHQAVKQAREYVKEGRRWVVDIDLEKFFDRVNHDILMARVARKVRDKRILRTIRQYLQAGIMTGGLVTARTEGTPQGSPLSPLLSNIMLDDWDKELERRGHRFCRYADDCNVYVHSKRAGERVMESLRQFLEKRLRLRVNTEKSRVDHPWQLKFLGYSMTWEKNTRLKVASQSVERFKSKLRLIFRQGRGRNVQIVINELNLVLRGWVQYFRLAEVKNIFEEMDGWIRRKLRCTLWKQWKRTYTRAKNLMKRGLEEVRAWQSATNGRGAWWNSAASHMNEAYPKSYFDKAGLVSLLQLLWQIQLT

SEQ ID NO: 17 - protein, 443 aa, synthetic construct
MSEPMEDKPEAVSERIKRAGELLRHWMWTEPTVWTERMLTALAQGVKGGKWFSLIDKIHPERTLRAAYGHVAANKGSAGVDHVTTTMFGDHLDEEVSKLSEQLRNGSYRPQAIRRHYIPKPGSQEKRPLGIPTVRDRVVQTALRMVIEPIFEREFAEDSYGFRPGRGCKDALRRVDELLKDGYIYIIDADLKSYFDTIPHDRLMDLVRQKISDGRVLNLIEAFLKQGILENLQEWIPESGSPQGAVVSPLLSNIYLDPLDHKMAQEGYQMVRYADDFVILCRTPEKAERALAAVQEWTAAAGLTLHPTKTRIVNATIDSFEFLGYRFSKGKRFPRTKSMQKLKDTIRAKTKRTNGQCLPAIIGSLNPTLRGWFEYFKHSYKTTFPELDGWIRGRLRSILRKRQKKRGRGRGSDHQRWPNAYFADLGLYSLKTAHKLACQSSSR

SEQ ID NO: 18 - protein, 430 aa, synthetic construct
MKGRMQKKSIDTCQQKNRTESDGYVEGQTFIWITENNLTDKYQLGNGLLEFILSPNNLNQAYHQVKRNKGAGGIDKMEVESLKDYLVRHKDELIKSILEGKYRPSPVRRVEIPKETGKTRQLGIPTVVDRVIQQSISQVLSQIYEKQFSDTSYGFRPRRGAHQALRTCQKNITEGYVWSVDMDLEKFFDTVNHSKLIEVLSRTIKDGRVISLIHKYLNAGVIVRNNFEETEVGVPQGGPLSPLLSNIMLNELDREIENRGHRFVRYADDMVILCKSKRSAVRTLENLLPFIEGKLFLKVNKEKTSVAQISKIKFLGYSFYRLNGECQLRVHPKSVLKMREKMKSLTSRSNGWGNERRKESLRQYIVGWVNYFKLANMKKLLLRTDEWYRRRLRMVIWKQWKRISTKLINLIKLGINKYKAWELVNTRKGY

SEQ ID NO: 19 - protein, 428 aa, synthetic construct
MSLATPERIRSLQRKLYCKAKAEPAFRFYTLYDKICRADILAHAYEVARSNAGAPGVDGRTFEHIEALGLQEWLAGLREDLVARTYRPMPVRRVMIPKPGGGERPLGIPTIRDRVIQTAAKLVLEPIFEADFEDSAYGYRPRRGASDAIKEVHRLLCRGHTDVADADLSKYFDTIPHAQLLRCVARRIVDGEVLRLIKLWLKTPIEGRDADGKRRLTGGKRSRCGTPQGGVVSPLLANLYMNRFLKHWRFTGCGEVFRAQIIAYADDFVILSCGHAAEAMMWTKAVMTKLGLNLNEAKTSIRDARKERFEFLGYSFGPHYWWKNGQRYLGASPSKKSVQRIKAKISELLVPGNKGSWPDVRDQLNRLLSGWSAYFNHGTRVPAYRAVDAHVYDRVQNFLCRRHNVRRRGTSRFSFGDVFGELAVLHLM

SEQ ID NO: 20 - protein, 400 aa, synthetic construct
MDEDFLTEAFYQLRKDAAAGIDEMTATEYEVNLGGRIADLHRRLVAQEYRAQPARRVWIPKGDGGQRPLAILVLEDKIVQRAVAMLLEAIYEPHFCGFSYGFRREHNAHQALTYLRQQCLELGINWIIDADIQKFFDTISWEQLRAILQRRMNDGAVLRLIGMWLHVGVLEAGQVINQELGTPQGAPISPILANIFLHIVLDEWFQSQVRPRMRGNCFLVRYADDFVMGYSLKGDAERVYEVLPKRFERYGLRIHPEKSRMVQFSRPYWQRGKGPGSFAFLGFTHYWAKTRSGSWTIKRKTQGKRLSRFLSGIADWCKENLHEAIGEQHRILSAKLRGHYQYYGVRGNFKLLEVAYEHTRGMWKKWLGRRNSMNRMSWEKFVEQVESVFTLPLPRIVHAF

SEQ ID NO: 21 - protein, 424 aa, synthetic construct
MPKTFYSLYDRLLHRRALARAFAKVRRAKGAKTPGQDGQTAADFAADEEAELDRLVHELRTKTYRPQPVRRVTIPKPGGGERNLGIPAVRDRVVQQSLLDILQPIFDPDFHPSSYGYRPGRSSQQAVAKATRFVRQYGLAHAVDMDLSKCFDRLDHDLIVASFRRRVRDGSILALMRMFLESGVMIGDDWEATEVGSPQGGVISPLISNVYLDAFDQEMKRRGYRIVRYADDILILCRSKSAALHAREVATAILEGPLRLTVNKEKTHLVHASQGVKFLGVEIGLTWTRIQDKKVAAFKAQVKRLTRRNSPVNLGQVIADLNPVLRGFANYFRMANCKGLLSELMSWIRRRLRAKQLALWKKPGRLHRRLRQLGYQGDFPKIRMTRWRNSRSLQASWSLPNGWFRELGLYELTAVETGVLPQAT

SEQ ID NO: 22 - protein, 427 aa, synthetic construct
MSELLEKILSKDNMNTAYKRVCANKGAGGVDDVTVEELGAYVKENWESIREQIRRREYTPQPVRRVEIPKPDGGERKLGIPTVMDRVIQQGIVQIISPMCEPLFSKWSYGFRPNRSCEMAIRQLLEYLNEGYEWIVDIDLEKFFDNVPQDRLMSLVHDIIKDGDTESLIRKYLKAGAMTPQGYKETKLGTPQGGNLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 23 - protein, 419 aa, synthetic construct
MEQVVERSNMLAALKRVERNGGAPGIDGVPTERLRDQIRAEWRRIREELLAGTYRPEPVRRVEIPKPGGGERLLGIPTVMDRLIQQALLQVLTPIFDPQFSDSSYGFRPGRRAHDAVKRARQYVEEGYEWAVDLDIEKFFDRVNHDILMARVARRVTDKRVLTLIRRYLQAGVMVNGVVMETAEGTPQGGPLSPLLANILLDDLDKELEKRGHKFVRYADDCNIYVKSKRAGERVMASVRNFLQERLKLKINEQKSAVDRPWKLKFLGFSMYKPKGGVILIRLASQTIDRVKAKIRGITARNKPISMDERIERLNTYLGGWIGYFALADTPSVFKDIEGWMRRRLRMCLWKQWKRVRTRYRELRALGLPEWVVHHFANARKGPWRMAHGPMNRALGNAYWQSHGLMSLTERYHRLRQAW

SEQ ID NO: 24 - protein, 427 aa, synthetic construct
MSELLERILSNDNMNAAYKRVCANKGAGGVDGVTVEELGDYIKENWSSIREQIRRRQYKPQPVRRVEIPKPNGGVRNLGIPTVMDRVIQQGIVQVLSPMCEPLFSERSYGFRPNRSCEMAIRQLLVYLNEGCEWIVDIDLEKFFDNVPQDRLMSLVHDIINDGDTESLIRKYLKAGVMTPQGYEETKLGTPQGGNLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 25 - protein, 502 aa, synthetic construct
VKPREAGMGGPSSSLAQVQTPSCEEGDSLLEKMLERENLLLALRRVEANKGAPGVDGVTVAQLRSYIQTHWADIRQQLLAGTYKPQPVRRVEIPKPGGGVRGLGIPTVIDRFIQQALLQVLNPIFDPEFSDNSFGFRPGRSAHDAVRRAQQYIQQGYRWAVDLDLAKFFDRVNHDKLMARVARKVKDKRVLKLIRAYLNAGIMADGVVVRNEEGTPQGGPLSPLLANIMLDDFDKELKKRGHRFVRYADDCNVYVKSRRAGERVMASLTRYLEGTLKLQVNQEKSAVDRPWKLKFLGFSFLPDKLATIRLAPKTLERFKERVRQITSRSRSMPIAERIRQLNAYIMGWVAYYRLAEMKRHCERFDEWIRRRLRMCIWKQWKRVRTRYRELRALGQPEWVVHMTANSRRGPWFMARMLNQAMDKTYFESTGTAQSSGEIPYTSWCFMNRRMRTRTYGGVRGRGLGAPSYSIGGTSTRCIVAHRIVVTFGQCAPSGFTEVSGGR

SEQ ID NO: 26 - protein, 430 aa, synthetic construct
MEQLGGDPRYCLMEQVVERANMTKALRRVERNGGAAGVDGMEPEALRSYLKEHWPRIKEELLAGTYRPMPVRRVEIPKPGGGVRLLGIPTVLDRLIQQALLQVLTPIFDPGFSPHSYDFRPGRSAHQAVEQARRYVAQGYRHVVDLDLAQFFDRVNHDLLMARVARKVKDKRVLKLIRAYLQAGVMINGCCVRTEEGTPQGGPLSPLLANIILDDLDKELERRGHKFVRYADDSNVYVRSCRAGQRVFESLKRFLEQRLKLRINEEKSAVDYAWRRGILGFSFTWEKEPRIRLAPQTVKRFKQRIRWLTKRSRSVNMAERLARLNEYLKGWMGYFRLIETPSTLQALDEWIRRRLRMCLLKQWKRPRTRRRNLVALGIPEDWARHISGSRKGCWRLANTPQVNKALGLAYWRSQGLVSLVDRYRELRSAS

SEQ ID NO: 27 - protein, 470 aa, synthetic construct
VRSREGRRQPKTLNRNCPREEVVKPRGIVGEPSPSPAQSGTSPRGDQGDSLMEKVVARDNMLAALKRVEQNKGAPGVDGIPTENLREQIRAEWPRIREELLAGTYRPQPVRRVEIPKPGGGKRMLGIPTVMDRLIQQALLQVLTPIFDPEFSEASYGFRPGRRAHDAVKKARQYVEEGYEWGVDMDLEKFFDRVNHDVLMARVARKVTDKRVLKVIRRYLQAGIVVNGVVMEREEGTPQGGPLSPLLANILLDDLDKELEKRGHKFVRYADDANIYVRSRRAGERVLASVRKFLQERLKLKLNEQKSTVDRPWKLKFLGFSMYKPKAGEIRIRLARQSIDRVKTKIRALTARNTPIAMEERIRRLNTYLGGWIGYFALAETPSIFEEIDGWIRRRLRMCLWKQWKRVRTRYRELRALGLPEWVVHPFANARKGPWRMAHGPMNRALGNAYWQAQGLMSLTERYRRLRQAW

SEQ ID NO: 28 - protein, 425 aa, synthetic construct
MEKQSMIINNILSNDNLNHAYKQVVKNKGAAGIDGMECEALFSHLRTNGAQLKESIRNQAYNPLPVKRVEISKEDGSKRNLGIPTVTDRLIQQAVAQVLTPIYEKVFHRNSYGFRPGKSAQQAVLKAVEYMNGGYNWVVDIDLEKFFDTVNHNKLISILNKEIKDGKVLSLIRKFLVSGVMVGEHVEETEIGTPQGGNLSPLLANIILNELDWELEKRKLKFVRYADDCIILVRSQKAAQRVMDSISKYIESKLLLKVNRKKSKIGRPIEIKYLGFTFYNQFKAKKYKAKAHEKSVQKVVRKLKQLTSRRWGVSNSVKAQKIAEVLRGWINYFKIGSILTITRKLDTMLRYRFRMCIWKHWKTPQNKYKNLVKLGVEPRNARRAAWSHGYARICRTEAVCYAMSNARLVKFGLLSAEAYFVKVSS

SEQ ID NO: 29 - protein, 427 aa, synthetic construct
MHEPTLLSQIIHPANLNQAYKQVMKNKGAPGIDDMPITALKAHLALHKETLVHQLQRREYKPQPVKRVEIPKASGGVRLLGIPTVTDRFIQQAIAQVLTPIFDKQFHDNSYGFRPKRYAEMAILQALENMNEGYEWLVDIDLERFFDTVHHDRLMNIVARTVTDGDVISLVRKFLVSGVMVQDEYQETIIGTPQGGNLSPLLSNIMLHELDKELANRDLRFVRYADDCLIFVKSDMAARRVMRSVSKFIEEKLGLIVNVTKSKVRRPEDEETKFLGFGFYYDWNDRLYKARPHKTSLQAIKFKLKCLTRRNWGVPTKSRVERINSLLRGWVNYFKIGKMKKHLIQLDANIRVRLRMCIWKQWKTPKNRRKNLIKLGMNRYEAYKYSYTSKSYARVAYSWILTTTITNQRLTKFGLISATEHYSNIHV

SEQ ID NO: 30 - protein, 425 aa, synthetic construct
MLDRDNLRLAYKRVVRNGGAPGVDGVTVAALQSYLNTHWDRVKNELLAGTYRPAPVRRVEIPKPGGGVRLLGIPTVMDRFLQQALLQVMNPIFDAHFSWYSYGFRPGKRAHDAVMQAQRYIRDGYRWVVDLDLEKFFDRVNHDMLMARVARKVKDKRVLKLIRAYLNAGVMINGVCQRTEEGTPQGGPLSPLLANILLDDLDKELTRRGLRFVRYADDCNIFVASKRAGERVMESAIRFLEGKLKLKVNRDKSAVDRPWKRKFLGFSFLSDKEATIRLAPKTISRFKERVREITSRSRPIPMEERIRRLNQYTMGWVSYFRLASMKNHCERFDQWIRRRLRMCLWKQWKRVRTRIRELRALGVPDWACFAMANSRRGPWEMSRNINNALPTSYWEAKGLKSMLTRYMVLRQPFGTAWCGPACQVV

SEQ ID NO: 31 - protein, 781 aa, synthetic construct
VHTHEDRIALSSPPNQADLQIHTGTLSPYTSHWGYSFLDPSIQEAPLLGQSEGQFLPPNLFSYPSGTLSSQEMPDPSPSTPELKVLPNTLKYVFLGPNETLPVIISSKLTMEQECKLTKVLLEHKAAIGWSLSDLSGISPSYCTHRIYTEEEARPSREMQRRLNPNMREVVKNEIIKWLDAGIIYPISDSPWVSPIHVVPKKAGLTIRVNDRGESIPTRTPTSWRVCVDYRKLNAATKKDHFPLPFIDQILERLAGNKFFCFLDGFSGYNQVAIDPRDQEKTTFTCPFGTFTFRRMPFGLCNAPATFQRCMLAIFHDMVGDFLEVFMDDFSVFGPSFDECLANLTRILRRCIDVNLVLSWEKSHFMVHEGIVLGHIISDRGIEVDRAKLDIIADLPVPGSVKQIRSFLGHVGFYRRFIKDFSKISRPLTHLLSTDVPFDMGPDCIQAFTQLRNAVTKAPVLQPPDWSLNFELMCDASDLAVGAILGQRRDRHPFVVYYASKTLDEAQRNYTTTEKELLAVVFGLEKFRSYLLGSHVIVFTDHAAIRHLMHKKDAKPRLIRWILLLQEFDIEIRDKVGAQNVVADHLSRLDPDHIGLTLPINEVFPDEHLLAINVASGPWYADIANYLASGSLPRGWPKNQRERLMAEAKYYFWDNPLLFRIGADQIIRRCIPDSEFPSVLSFCHDQACGGHFSGRKTAAKVLQCGFFWPSLHQDAHTYAKHCTRCQSLGSMARRDMMPLQHILAAEIFDIWGIDFMGPFPPSNGFEFILVAVDYVSKWIEA

SEQ ID NO: 32 - protein, 761 aa, synthetic construct
ENPIKIDFFEPLEIEPLNLIDVNFVEKMDMDIDLSYEQDSLLKQNLKNLRLDHCNKEEKDAIRKLCFDYRDIFYCEQIPLSFTSEITHKIKLNDESPIYTKSYRFPEIHKKEVKDQIAKMLDQGIIQHSISPWSSPIWVVPKKLDASGRRKWRLVIDYRKLNEKSCNDRYPLPNITDILDKLGRANYFTTLDLASGFHQIQVDPDDIPKTAFSTEGGHYEFKRMPFGLKNAPSTFQRVMDNILRGLHNEICLVYLDDIIIFSTSLDEHIQRLKSVFDRLRKSNFKLQLDKSEFLQKTVQYLGHIITPQGVKPNPDKVSTIKRFPIPRTQKDIKSFLGLLGYYRRFIKDFAKITKPLTKCLKKNAKVTHDQNFIDAFNTCKEILVNDPILQYPDFSKPFILTTDASDVALGAILSQGTLPNDRPVAYASRTLNETESKYSTIEKELLAIVWACKHFRPYLYGRKFTIYTDHRPLTWLFSLKEPNSKLVRWRLKLEEFEYDIIYKKGKLNTNADCLSRVTLNAIDNESMLNNPGDIDQDILDTLQNVTQNLQTLQDFQKPSTSNTNDKINIISDIQIKPPDNSQNDNTNTSTQHSTANETTNDGIKIFDEIINNKTNQILVFPNVIYKLDIKRETYENHKIVTVKIPIINNEQMILQFLKENTDPKHVYCMYFQSDELYNDFCTVYLKHFSEKGPKLIRCLKLVNTVADKEEQILLIKNHHGSKTNHRGINETLEKLKFNYYWKNMKSTVSNFINACDICQRA

SEQ ID NO: 33 - protein, 799 aa, synthetic construct
MIAMDDEEWGEEDIREFTKLVENSEKAWEPASEKLEVINLGNKQEKKELKIGTLVTTEERNKLVSLLHEYADVFAWTYADMPGLDTDIVVHKIPLIEGSKPVKQKTRRMRPDILLKVKSEIQKQWDAGFLDVVQYPQWVANVVVVPKKDGKIRVCVDYRDLNKASPKDDFPLPHIDILVDNAARNATYSFMDGFSGYNQIRMAEEDKEKTTFVTPWGTFCYKVMPFGLKNAGATYQRAMVTLFHDMMHREVEVYVDDILAKSKDEEDHVQVLRRLFERLRKYQLKLNPAKCSFGVKTGKLLGFIVSGRGIEVDPDKAKAIQEMPAPKTEKKVRSFLGRLNYIARFISQLTVTCEPIFRLLRKKNPGVWDNDCQEAFDKIKRYLQNPPLLVPPTPGRPLILYLTVTETAMGCVLGQHDESGRKEQAIYYLSKKFNDCESRYTTIERLCCALVWSAKRLRQYMLYYTTWLISKLDPLKYICEKPYLSSRIARWQVLLAEYDIVFMTRKAVKGSIIADHLADHAMENYEPLNFDLPDEDVIVIENEGGESDQWTLYFDGAVNVSGNGAGAVVISPENKQYPVSTRLLFECTNNTAEYEACIIGLEAALELKAKKLEVFGDSLLIICQVKGEWQTKDEKLKLYQNYLLRLANEFEEIKFTHISRDKNQFADALATLASMTQVNAKSKIQPIDIEVRSFQAHCCLIEDSLDGKPWYNDIKKFLQHREYPQGISKTDEKTLRRMTMNFYLDGEILYKRSFDGTLLRCLNENEIEQALKEVHEGICAAHANGHTMAKQMQRSGYSG

SEQ ID NO: 34 - protein, 553 aa, synthetic construct
MDIPVRPVRQRRRKFNEERRLVVKEETQKLLSAGHIREIQYPEWLANVVLVKKANGKWRMCVDFTDLNKASPKDSYPLSSIDALVDSASGCEMLSFLDAFSGYNQIKMHPRDEGKTTFMMETCSYCYKVMPFGLKNAGATYQRLMDKVLAPMLGRNVQAYVDDMVVASHDRRRHTADLEELFVTISKYRLKLNPKKCVFGVEAGKFLGFMLTKRGIEANPDKCAAIIAMRSPTSVKEVQQLTGRMTALSRFVSAGGEKGYHYFQCLKRNSRFAWTDECEAAFVKLKEYLATSPVLCKPVASVPLRLYFAVTERAISSVLVQERNQVQRPIYFVIKALQGPETRYQSLEKAALAVVFSARRLRHYFHSFTIVVMINLPIQKVLQKPDVAGRMVRWAVELSEFDIQYEPRGSIKGQVYADFVAELSPGGEQEVESGSQWLLSVDGSSNQQGSGAGIVLEGPNGVLIEQALRFAFKASNNQDEYEALIAGMLLAKEMGARNLLVKSDSQLITGKVLGEFQAKDPQMAAYLRYVQLLKGAFSALELVHVPREQNARA

SEQ ID NO: 35 - protein, 707 aa, synthetic construct
MGSFLLVPEADYNLLGRDLMIELGINLEVRNKEIAVKLYALTAEDEARINPEVWYTPESVGKLDITPFEITIMNPEIPIRVKQYPMSQEGKKGLQSVIQRLLKQGLLEPCMSPYNTPILPIKKPDGSYRLVQDLREVNKRTLTRFPVVANPYTLLSQLSPELKWYSVIDLKDAFWACPLKEECRNYFAFEWEDPETHPKQQFRWTVLPQGFTESSNLFGQALDQVLSTYELNPEVTLVQYVDDLLLAGENQEAVREESIKLLNFLTLKGLKVSKSKLQFTEEEVKYLGHWLSKGTKKLDPERVKGILSLQAPKSKRQVRQLLGLLGYCRQWIENYSKKVKFLYQKLVREGLIKWSQTDKKCFEDLKADLVNAPVLSLPDTKKPFYLFVNTEEGMAYGVLTQKWADRKKLIGYFSKLLDPVSRGWPTCLQAIVATALLVEEAGKVTLGGELKVYTPHNIRGVLQQKAKKWITDARLLKYEGILIHSPKLEIETTSLQNPAQFLYGEPSEELTHDCVRLIESQTKIREDLEEEELPSGEKLFVDGSLRVVNGKRKSVYAIIDGNTSEVKESGPLDTTWSAQACELFAVLKALQLLKGKVGSIFTDSRYAYGVVNTFGKIWEERGLINTQGKNLVHETLIRQTLEALRGPRQVAVVHVKGHQKGASVQIRGNNLADEEAKRAALLIVKETEKPREATGNSKSFTARGIEK

SEQ ID NO: 36 - protein, 753 aa, synthetic construct
VGGQSVDFLLDTGATYSVLTEAPGPLSSRSASVMGLSGRAKRYYFSYSLSCNWDSVLFSHEFLIVPESPSPLLGRDILSKVHASVFMNMEPSLSLPLVEQNVNPRVWADGKSVGRAQNAIPVVVKLKDPHVFPHKKQYPLKPEVKEGLKPIIENLKEQGLLIPCNSPCNTPILGIKKSNGKWRLVQDLQIINEAVVPLHPVVPNPYTLLSEIPERAKYFSVIDLKDAFYSVPLAEESQFLFAFEDPTQPASQLTWTVLPQGFRDSPHLFGQSLSRDLQNFNSSEAVVLQYVDDILLCAETEEACSRASEDFLNFLAGCGYKASREKAQLCQQSVRYLGLIISEGTRAIGPERIKPILNHPLPMTLRQLRGFLGITGYCRIWIPGYGELARPLYKLIAETQQAQTDKLVWSPETQKAFKVLQTALLQAPALSLPTGSEFNLFVTERKGVALGVLTQPRGPHQQPIAYLSRELDVVSRGWPHCLRVIGAAALLAPEALKIINGRNLTVLTSHDVSGILNSKVNIWMTDSRLLKYQSLLLEGPVTKLKVCGNLNPATFLPEKENETPDHDCSQFLTLNYAAREDLKDTPLDNPDMEIFTDGSSFVRDGKRKAGYAVVTAEQVLEAKSLPQGTSAQLAELVALTRALELSKGQRMGLIIYVSLLLLTPKILSLPLDPQDNVFLSWAHSYAAFHNRSNCWVCGALPSSSVEGFPWWTSPLQGKDFLQVCEYLRQQSHAMPLLHLMTSTNPKMDWCNTL

SEQ ID NO: 37 - protein, 771 aa, synthetic construct
MLSEEALEVLKTVPPCVWSQGKCDVGRMVNAKPVHVRPKTNYRPNIRQYPLKEDAKVGIKPVIEEMLKAGILRECPDASCNTPIFPVKKADGQSWRMIQDLRAVNDAVQSTAPTVPDPHTLMNGLKPNMAWFSVIDLSNAFFSIPVEKDSQGWFGFTHEGTKYTYTRLPQGYVDSPTIFSREIANCLATLQIPDTSQILVYVDDILVASPTQEESTKVTLQVLTHLAKTGNKVSRQKLQFVKEEVIFLGHNLNREGRRIHSERKQTVLNEPKPTTKKQMMQFLGLTNYCRAWIPDYAGMTQPLLDCIYSDNDMKMSGKIEWTQEAEAAFESTKQCLVGSSVLALPDYEKPFVQTVDCKKGFMTSVLAQKHGTKLRPVAYYSKRLDPVAQALPVCVQAVCAAAMAVHCTAEIVLFHPLTLMVPHAVTMLLHDTKMAFLSPARYLALTATLMSQPHIVIKRCNILNPATLIPTAEDGEPHCCKEETDRTCKPRPDLKDIQLLSGETWFVDGSCSKSITGQNQTGFAVVSHSQVIKAGRLPHTYSAQAAELVALTEACKAGVGKDVTMWTDSQYAHSTVHIFAAQWARRGMKTSTGKPVTHAQLLTDLLKAVLLPKSIAICKCAAHTSGKDAVTLGNAHADKVAKLAAMGEYGFHILLQKGESVSQPIPLVILRDMQNSAPDREKKKWLTDGATTDPEGTFRINNKIVLPVSLYKTAAHLSHGPCHVSTGGMVTIINEHFHTYNYITFSKNFCRACVVCCRHNAQGNERPQRGK

SEQ ID NO: 38 - protein, 698 aa, synthetic construct
MVMALLAATSAPTVPTVPVELSDISETLWTTISTETGLLKSAEPVYITYHTDIPLPRKMQYPLPKEAIEGIQPVLQQLLTQGIIKEATSPCNTPILPVKKPKPGSYGKPVYRFVHDLRLINAIVIPRAPVIPNPATVLTAIPPEATYFTVIDLSSAFFSNPVHPDSQFLFAFTFDGRQYIWTRLPQGYRESPTIFSQYLKWDLDSIRCAMGSTLIQYVDDLLLCSTAKDACVQDTRILLFALAEKGHKVVLSKLQFAQSEVEYLWHRISYVSTALTQDRIESVLSKPRPTTKKQLHQLLGASGYCRAWIPAYVELVHPLTDLLLDSIPEPLPWTTLHDNALTALKQALTSAPALGRPDYAKPFTLFCHECYGTASGVLTQPFGPGQRMVAYFSVLLDPCAQGLPPCLRAVGAVAMLVETALGLTLGHPLLVQVPHAVAALLNRGSTQHLTAARLTKYELALLANQTITLHRCEVLNPATLLPLPEDGMPHNCLHLMDLISTPRPDLSDVPLQNPDLQLFCDGSAHQDEFGALRVGYAIVILMTTLEAHSLPTQKSAQAAELVALVRACQLAENKTVAIYTDSKYAFSVCHSTGQLWKHHGFLTSCGSQISHAALISALLEAIQLPAAVSVTHCDFVCVKIHRRKHCLEPWWSAPAQVLLTTPTAVKIEGIDRWVHCSHCKKVPAPTSPTTAITTDALT

SEQ ID NO: 39 - protein, 770 aa, synthetic construct
VFAWDAYDAPGVDPSLICHHLNVNPSSTPKKQPPRRPSKEHASAVKDEVAKLKRAGAIKEVFYPEWLANTVVVKKKTGKWRVCVDFTDLNKACPKDPFPLPRIDRLVDATVGHPRMSFLDAFQGYHQIPLALEDQEKTAFMTPIGNYHYKVMPFGLKNAGSTYQRMMTRMFEPQLGKIIEVYIDDMVVKSRRASEHVKDLGTIFTILREHRLRLNASKCSFGVGSGKFLGYMVTHRGIEVSPDQIKAINSLQAPRNPKEVQKLTGMIAALNRFISRSADRCRPFYLLINKWKEFAWSEDCVQAFQQLKDYLSRPPIMSSPEADEVLFAYIAVAPHAVSLVLIRDDNGVQRPVYYVSKSLHEAEVRYLPLEKAILAIVHATRKLPHYFQAHTVIVLTQLPLRAVLRSADYTGRIAMWSALLGAFDIKYMPRSSIKGQVLADLVAEFAEPSIETITEKRDMDGKSVGAISTGRNTHWKVYVDGAANQRGSGVGIVLISPDGAAIEKSLRLGFSATNNEAEYEALLQGMTMVQRLGGKMIEAFSDSRLVVGQVMGELEARDTRMQEYLGQVKRLQTNFESFNLTHISRSVNTHADSLATLATSSAHNLPRVILVEDLAQASPISRGPARVHQIRKSHSWMDPIKNFLQNDVLPEENWKPRRYEGMLLGFGCQRTTNYIGAPILDRTYSAYTQKNPNLCLRSYTRGFVEVIPEGDRWHTGHLPRGTGGRTCRERPKNTLRSAISAKDSPRISTNPEEFSTPSRVHGRSHNGALI

SEQ ID NO: 40 - protein, 655 aa, synthetic construct
VLIRDTSITTNPLPARYHDFSDVFEKKNADRLPEHRLYDCPIDLQEGAHPPFGPIYGLAEPELEALREYLKENLAKGFIQPSKSPAGAPILFVKKKDGSLRLCVDYRGLNRVTVRNRYPLPLIPELLDRLRTGRVFSKIDLRGAYNLVRIKPGDEWKTAFRTRYGHFEYKVMPFGLTNAPAVFQHMMNHIFREYLDHFVVIYLDDILVFSPSMEEHTRHVRLILTKLREHGLYAKSEKCEFDRTSVEFLGYVISPAGITMDPRKVATIHNWPIPTRLKEVQSFLGFANFYRRFIDRFSTIVQPLISLTRKDVPFVWTATTQHAFDALKQAFMSAPVLVHPNPAKPFQVETDASDFALGAILSQLDDDGTLHPVAYYSRKFLASEINYPVYDKELAAIISAFAEWRPYLVGAQHRIQVVTDHKNLLYFASSRTLNRRQARWSIFLADYDFEIIFRPGIQHGKADALSRRPDLALCPGDDAYTQQSCSLLKPDQLQLSATFMLHDDSLLQEIAQATKTDTFATEILKRLQDSSPATKRADLHHFTTHDGLLYRNHLLYVPEGPCRTQVLQTCHDDPLAGHFGVAKTLELVSRGFWWPQPWKLVKEFVKTCDVCARSKAVHHRPYGLLHPLPIPNRPWASISMDFITDLPPVNGVDTV

SEQ ID NO: 41 - protein, 631 aa, synthetic construct
VSAPDPPVPHADKIIWETQIPVWVDQWPLTQEKLKAASMLVQEQLAADHVEPSTSPWNTPIFVIKKKSGKWRLLQDLRAINRVMRPMGALQPGIPTPVAIPKDYYKMVIDLKDCFFTIPLHPEDRPYFAFSLPRINFQGPMQRFQWKVLPQGMANSPSLCQKYVAQAVDPVRKRWPQIYILHYMDDLLIAAPDTHNLNGCYLDLYKALTTLGLQIAPDKVQTQDPYTYLGLQLQGNQIQTPKIQLRVDHLHTLNDFQKLMGDIQWLRPYLKLPTCVLLPLNDILKGDSHPTSPRKLTPEAQQALNQVTKAITQQFVTQISYNLPLILVILHTPHTPTGIFWQRDPHFKNKGCPLFWVHLPTTSSKVITVYPAQVASVIVKGRLLSRQHFGKDPDKILIPYTKDQTQFLLQTEDEWSIALSGFLGTIDNHLPSDPLLHFAQLHPFIFPKITQKAPIPQALTVFTDGSSNGCAVFVVGNQPTTFETAYQSAQLVELFAIAQVFITFLDKPVNIYTDSAYIAHSVPLLEISPAIKASSNAAFLFHQLQQLIQTRRHPFFLGHIRAHSDLPGPLTQGNAQADAATRSIFPIFAGSIQRATELHTLHHLNSQSLRLFCKITRQQAREIVKACPACT

SEQ ID NO: 42 - protein, 578 aa, synthetic construct
VSAPDPPVPHADKIIWETQIPVWVDQWPLTQEKLKAASMLVQEQLAADHVEPSTSPWNTPIFVIKKKSGKWRLLQDLRAINRVMRPMGALQPGIPTPVAIPKDYYKMVIDLKDCFFTIPLHPEDRPYFAFSLPRINFQGPMQRFQWKVLPQGMANSPSLCQKYVAQAVDPVRKRWPQIYILHYMDDLLIATPDTHNLNGCYLDLYKALTTLGLQIAPDKVQTQDPYTYLGLQLQGNQIQTPKIQLRVDHLHTLNDFQKLMGDIQWLRPYLKLPTCVLLPLNDILKGDSHPTSPRKLTPEAQQALNQVTKAITQQFVTQISYNLPLILVILHTPHTPTGIFWQRDPHFKNKGCPLFWVHLPTTSSKVITVYPAQVASVIVKGRLLSRQHFGKDPDKILIPYTKDQTQFLLQTEDEWSIALSGFLGTIDNHLPSDPLLHFAQLHPFIFPKITQKAPIPQALTVFTDGSSNGCAVFVVGNQPTTFETAYQSAQLVELFAIAQVFITFLDKPVNIYTDSAYIAHSVPLLEISPAIKASSNAAFLFHQLQQLIQTRRHPFFLGHIRAHSDLPGPLTQGNAQAD

SEQ ID NO: 43 - protein, 609 aa, synthetic construct
VEEAAPIQGNNRDQQAANAMEREIGGKTFKLGRLLSQEKQEGVAEVISRHLDAFAWSASDMPGIDPDFLCHHLSMDVAVRPVRQRRRKYNEERQLVVKEETQKLLSAGHIRVIQYPEWLANVVLVKKANGKWRMCVDFTDLNKACPKDSCPLPSIDALVDSASSCKVLSFLDAFSGYNQIKMHPRDESKTAFMIETCSYCYKVMPFGLKNTGTTYQRLMDKVLAPMLGRNVYAYVDDMVVASQDREHHVADLEELFVTISKYRLKLNPEKCVFGVDAGKFLGFMLTERGKEANPNKCAVIIAMRSPTSVKEVQQLTGRMAALSRFVSAGGQKGHPYFQCLKRNSRFAWTDECEAAFIKLKEYLATPPVLCKSVTGVPLRLYFTVTERAISSVLVQEQDHSQKPISFVSKALQGAETRYQALEKAALAVVFSARRLRHYFHSFTVVVIANLPIQKVLQKPDVAGRMVRWAVELSEFDIQYEPRGSIKGQVYADFVAELSPGGEQEVEVGSQWLLSVDGSSNQQGSGAGIVLEGPNDVLIEQALRFAFKASNNQAEDEALIAGMLLAKEMVAQNLLVKSDSQLITGQVSGEFQAKDPQMAAYLKYVQLLKG

SEQ ID NO: 44 - protein, 767 aa, synthetic construct
VYRKQFKIPEAHQTFIEQTLDEWLKLGVVKRSNSLYNSPLFCVPKKQGRGLRIVQDFRELNNHSHIDKYSMKEITECIGDIGRANSSIFSTLDLTSGFWQMKLDEASQPLTAFTIPGQGQFQWITSPMGLLGCPASFQRLMETVLRGIKNVLVYIDDLLIHTATHEEHLIVLEQVFERLHKNHLKINLEKCVFGNPEVSYLGYTLTPNGIKPGRNKLQAIKDAQPPTNIKMVRSFVGLCNFFRTHIKDFAIIAAPLFKVTRKDSNYKSGQLPPDALHAFRVLQLQLTSEPVMAYPRSDRQYALITDAATGTAETPGGLGAILTQIDKSGNHFAISFASRQLKDHEKNYSPFLLEAAAAVWGMDVFNEYLRGKQFILFTDHKPLEKLGHLHTKTLNRLQTALLEHDFVIQYKKGAIMPADYLSRLPTLQVQNVEATVAAFDPFQPDLSKLQVPFQVNTAADVIAAFDPFQPNLKELQFQDTDLQAIYLFLKNGQWLPHLTKRQINSLSALSQKVFFDKNKLAWIRLDDYKYPRTALWLPEFYRKEALCESHNQIFAGHNAAQKTYLKLTSSYFWPNVYSHVLQHTQTCFRCQQRKSSRAKQPPLAPLPIPDLPNVRIHADLFGPMLGVEKKFAYVLCITDAFTKYAMVTKVDNKDAETVARAIFDNWFCKFGIPAQIHTDGGKEFVNKLSAELFELLNVQHSKTSPYHPQCNAQVEVFNKTVKKYLASYVDKTTLNWNDFLPALMLAYNTSYHSTIATTPFELLFGVK

SEQ ID NO: 45 - protein, 697 aa, synthetic construct
MKEDHRKIEELVPRKFLKWRKVFGKVESERMPTRKVWDHAIDLKEMFKPQKGRIYPLSKNKREEVQNFVEDQLRKGYIRPSKSPQTLPVFFVGKKDGSKWMVMDYHNLNNQTVKNNYLLPLITELIDNMGSKKVFTKMDLRWGFNNMRIKEGDEWKGVFTMHIGSFEPIVMFFGMTNLPATFQAMINEILRDLINKGKVAAFVDDVLVGTETEKGHDKIVEEILRRLEENDLYVKPEKCIWKVWKIGFLGVVMEPNGIEMEEEKVDRVLSWPELENVKDIRKFLGLANYYRRFIKDFARVARPINMLMRKDVKWQWGVEQQKAFDKLKRVFTTKLVLAAPDLDKEFRVEADVSNYATRGVLSMKCSDEMWRPIAFISKSLSDTERNYEIHDKKMLAVVRCLEAWRYFLEGATTKFEIWTDHKNLEYFMKAQKLNRRQARWALYLSRFNFMLKHVLGSKMGKADSLSRRPDWEVGVEKDNKDQKLVKPEWLEVRKTEMVEIIVDGVDLLEEVRKSKVRDDEVVKVVEEMKKAGVKILRDEEWREADGVMYKKEKVYVPKDNKLRAEIIRLHHDMPVGGHGRQWKMVELVTQNFWWPGITKEVKRYVEGCDACQHNKNRTEQPAGKLMPNSIPEKPWTHISVDFITKLPLVQGYDSILVVVDWLTKMVHFIPTMEKTSAEGLARLFRDNVWKLHGLLES

SEQ ID NO: 46 - protein, 777 aa, synthetic construct
MKEDHRKIEELVPRKFLKWRKVFGKVESERMPTRKVWDHAIDLKEMFKPQKGRIYPLSKNKREEVQNFVEDQLRKGYIRPSKSPQTLPVFFVGKKDGSKWMVMDYHNLNNQTVKNNYLLPLITELIDNMGSKKVFTKMDLRWGFNNMRIKEGDEWKGVFTMHIGSFEPIVMFFGMTNLPATFQAMINEILRDLINKGKVAAFVDDVLVGTETEKGHDKIVEEILRRLEENDLYVKPEKCIWKVWKIGFLGVVMEPNGIEMEEEKVDRVLSWPELENVKDIRKFLGLANYYRRFIKDFARVARPINMLMRKDVKWQWGVEQQKAFDKLKRVFTTKLVLAAPDLDKEFRVEADVSNYATRGVLSMKCSDEMWRPIAFISKSLSDTERNYEIHDKKMLAVVRCLEAWRYFLEGATTKFEIWTDHKNLEYFMKAQKLNRRQARWALYLSRFNFMLKHVLGSKMGKADSLSRRPDWEVGVEKDNKDQKLVKPEWLEVRKTEMVEIIVDGVDLLEEVRKSKVRDDEVVKVVEEMKKAGVKILRDEEWREADGVMYKKEKVYVPKDNKLRAEIIRLHHDMPVGGHGRQWKMVELVTQNFWWPGITKEVKRYVEGCDACQHNKNRTEQPAGKLMPNSIPEKPWTHISVDFITKLPLVQGYDSILVVVDWLTKMVHFIPTMEKTSAEGLARLFRDNVWKLHGLLESIISDRGPQFAAGLIRELNGILGIASKLLTVFHPQTNGQTERVNQELEQYLRMFINYRQEQWPEWLGTAEFAYNNKVHSST

SEQ ID NO: 47 - protein, 706 aa, synthetic construct
MVKIRGDLSSEMKQQLYNPLKEFKDIFAWSYKDMPGLDFEIVQHRLPLKPECHPIKQRLRRMKPEVSLKIKEEVEKQFNAGFLAVAQYPQWVANVVPVPKKDGSVRMCVDYRDLNRASPKDDFPLPHIDTLVDNTATSSLYSFMDGFSGYNQIKMTPEDMEKITFITLWGTFCYKVMSFGLKNAGATYQRAMMALFHDMMHKEIEVYVDDMIAKSESEEEHLVNLKKLFERLRKYKLRLNPSKCTFGVKSGKLLGFIVSQKGIEVDPDKVRAILEMPHPCTEKQVRGFLGRLNYIARFISQLTATCEPIFKLLRKNQVVKWNENCQNAFDKIKEYLKEPPILHPPVPGRPLILYLTVLDGSMGCVLGQHDGTGKKEHVIYYLSKKFTDCEQRYSSLEKTCCVLAWTAHRLRQYMLSHTTWLVSKMDPIKYIFEKPALTGRIARWQMLLSEFNIVYVIQKAIKGSALAEYLAQQPIDDYQPMQPEFPDEDIMTLFVSEDDGNERKWVLFFDGSSNALGHGIGAVLISPEKQYIPMTARLCFDCTNNIAEYEACAMGIRAAIEYKARRLNVYGDSALVIHQVKGEWETRDQKLIPYKAYIRGLVEYFDEIEFHHISREDNQLADALATLSSMFAISQEEELPMIKMQSHENPVYCNFIEEEVDGKPWYFDIKRYLQNREYPDSASENDKRMLRRLASSFILDGDVLYK

SEQ ID NO: 48 - protein, 706 aa, synthetic construct
MVKIRGDLSSEMKQQLYNPLKEFKDIFAWSYKDMPGLDFEIVQHRLPLKPECHPIKQRLRRMKPEVSLKIKEEVEKQFNAGFLAVAQYPQWVANVVPVPKKDGSVRMCVDYRDLNRASPKDDFPLPHIDTLVDNTATSSLYSFMDGFSGYNQIKMTPEDMEKITFITLWGTFCYKVMSFGLKNAGATYQRAMMALFHDMMHKEIEVYVDDMIAKSESEEEHLVNLKKLFERLRKYKLRLNPSKCTFGVKSGKLLGFIVSQKGIEVDPDKVRAILEMPHPCTEKQVRGFLGRLNYIARFISQLTATCEPIFKLLRKNQVVKWNENCQNAFDKIKEYLKEPPILHPPVPGRPLILYLTVLDGSMGCVLGQHDGTGKKEHVIYYLSKKFTDCEQRYSSLEKTCCVLAWTAHRLRQYMLSHTTWLVSKMDPIKYIFEKPALTGRIARWQMLLSEFNIVYVIQKAIKGSALAEYLAQQPIDDYQPMQPEFPDEDIMTLFVSEDDGNERKWILFFDGSSNALGHGIGAVLISPEKQYIPMTARLCFDCTNNIAEYEACAMGIRAAIEYKARRLNVYGDSALVIHQVKGEWETRDQKLIPYKAYIRGLVEYFDEIEFHHISREDNQLADALATLSSMFAISQEEELPMIKMQSHENPVYCNFIEEEVDGKPWYFDIKRYLQNREYPDSASENDKRMLRRLASSFILDGDVLYK

SEQ ID NO: 49 - protein, 688 aa, synthetic construct
MKYPYSNVYSITCYDQIDKCVQQVCDFDCEDGLSITLSYGYDFTKIEEMERHVCVPQNVHESVLALQALQTVPHGNLFVDLVLSHKKLLPSILQAPELELKPLPDNLKYVFIGDNNTLPVIIAKGLTNIQEEKLVKLLCDHKTAIGWTLADIKGISPSMCMHHILLEDNAKPTREMQRRLNPPMMEVVKAEILKLLDAGVIYPITDSKWVAPIHVVPKKTGITLVKNKNDELIPTRISSGWRMCVDYRKLNLSTRKDHFPLPFMDQMLERLAGKSFYCFLDGYSGYNQIVINPEDQEKTTFTCPFGTYAYRRMPFGLCNAPATFQRCMMSIFSDYVERIIEVFMDDFTVYGDSFDKCLENLSLILKRCIETNLVLNYEKCYFMVEQGIVLGHVVSSRGLEVDKAKIDVISSLPYPSCVREVRSFLGHAGFYRRFIKDFSKITAPLCRLLAKEVDFVFDQACKDAHDELKRRVTSAPIIQPPNWDEPFEIMCDASDYAVGAVLGQRIGKNLHVIAYASRMLDGAQCNYHTTEKELFAVVFALEKFRSYLLGTKVIVFTDHAALRYLLKKKESKPRLIRWILLLQEFDLEIKDKKGAENHVADHLSRLRTEDIQIETIRETFPDEQLYMLHSSTRPWYADLVNYLVTKEFPPGLSKPQKDKLRADAKYYFWDDPYLWKFCADQVVRRCVP

SEQ ID NO: 50 - protein, 527 aa, synthetic construct
MPGVPRNLIEHSLNVSKTARPIKQKLRRFARDKKEAIRAEITRLLAAGFIKEVYHPDWLANPVLVRKKNNEWRMCVDYTDLNKHCPKDPFGLPRIDEVVDSTAGCELLSFLDCYSGYHQISLKEEDQIKTSFITPFGAYCYTTMSFGLKNAGATYQRAIQQCLHDEIRDDLVEAYVDDVVVKTRDASTLIDNLDRTFKALNRYKWKLNPKKCIFGVPSGLLLGNVVSRDGIRPNPSKVKAVLDMRPPKNVKDIQKLTGCMAALSRFISRLGEKGLPFFKLLKASEKFSWTEEADTAFNQLKTFLTSPPVLTAPQPNEDLLLYIAATDRVVSTAIVVERDEPGHVFKVQRPVYFISEVLNESKARYPQIQKLIYAILITSRKLKHYFDGHRVLVTTSFPLGDILRNKDANGRIVKWAMELCPFSLEFQSRTTVKSQALVDFIAEWTDLNEPSVPDVSDHWSMFFDGSLNINGAGAGILFVSPNKDKLRYVLRILFPASNNVAEYEACLHGIRLAVELGVKRLYVYGDS

SEQ ID NO: 51 - protein, 572 aa, synthetic construct
VPGNEDLDFRKQISPELDEEETEKLVKALGRFVDVFAKGNEPLGMCNMAEHEIPLVPNAKPVYQSLGSGAYKEQLIHRELMSKMKARGAIEVSVGPWGARVLLAQKKDGTWRFCVDYRLLNALTVNSVYPMPSIDGTLARLHGAKIFSIMDLECGYWQIPIKKEDRVKTAFITADGVFQFICMPFGLCTAPSTFQRTMDLVLGGLRWSVCLVYLDDIIVYARNTEEHRQRLVMVLSALRKANLKLKLAKCRFAEKEITALGHRISAEGVRPDPDKVRAVKEFPGLPTSGKRADLVKWVKSFLGLVSYYRRFFPGFAEVAKPLMDLTKDKTLFIWGASHQKSFDELKERLANAAVQAYPDYSLEFEIHPDACGYGVGAVLVQRQMGEERPLAFASRIMTASERNYSITEKECLALVWAVKKFRFFIWGRPIRIVTDHHALCWLQSKKDLAGRLSRWAMQLMEYKYTIAHKDGRLHADADALSRYPLALGLGSDATDKESRDLVVNVVTQCDRSELQEGQRSEWAYVFNNHEQEKETANYTIRNGLLFRIRLWMASRGKLRCDSACRRPCGRGY

SEQ ID NO: 52 - protein, 277 aa, synthetic construct
MSLMEEVVSRENMQAAYRRVVRNRGAAGPDGMTVEDLADHCRQHWPRIREELLAGRYRPQPVRRVTIPKPGGGERALGIPTVLDRMIQQALLQVLQPVFDPRFSPDSYGFRPGRGAHDAVLRARDHVRGGRRWVVDLDLEKFFDRVNHDVLMSRVARRIEDKGVLRLIRRFLQAGVLEGGVASPRLEGTPQGGPLSPLLSNILLDEWDKELERRGHRFVRYADDCQIYVQSQRAGERVMRSMTRFLEEVLRLRVNRVKSAVARPWERQFLGYGFTQH

SEQ ID NO: 53 - protein, 298 aa, synthetic construct
MEIETPQRLRKLQQVLHRNAKANSKWRAWSLYADLLREDVLAHAAAKVIANKGAAGIDGTSVDDIRSSESRERFLSDLREELSSKAYKPSPVLCVRIPKKDGKTRALGIPTVKDRVVQPALVLLLEPIFEADMHAESYGYRKGKNAHQAMDSICRALYSGRHEVIDADISGYFDNIDHAMLMKLVKRRVSDGSILKLIKSFLTAPVVENGTGRKNDKGTPQGGVLSPLLANLYLNGLDHQVNGKAGQGAQMVRYADDLVILCHRGKGAELLERLDRYLATKGLSLNRRKTRRVDFTKE

SEQ ID NO: 54 - protein, 345 aa, synthetic construct
LGIPTLTDRLIQQALHQVLSPIFEAEFSESSYGFRPGRNAHQAVKAAKQYVAEGRRFVVDMDLEKFFDRVNHDVLMAKVAKKVEDGRVLKLIRRYLEAGMMAEGVVSPRTQGTPQGGPLSPLLSNILLTDLDRELERRGLAFCRYADDCNIYVRSQAAGERVLAGISRFLSERLKLRVNEAKSAVARPWERKFLGYSLTWHKTPKLRIAPASLQRLKGKIRKMLKGASGRSVRRTIEELNPVLRGWAAYFKLAETKSALEELDGWLRRKLRCILWRQWKRPYTRARNLMKAGLKEERAFRSAFNQRGPWWNSGASHMNAAFRNVYFDRLGLVSLLDTTRRLQYLS

SEQ ID NO: 55 - protein, 651 aa, synthetic construct
EYKVSIAPIPEYILGIDIMSGLTLQTTVGEFRLRERCISIRAVQAIIRGHAKIEPIFLPQPRCITNTKQYRLSGGEEEFTKIVQELERVGIIRPAHSPYNSPIWPVRKPDGTWRMTVDYRELNKVTPPIHAAVPNIASLMDTLSREIETYHCVLDLANAFFSIPIAKESQDQFAFTWEGRQWTFQVLPQGYVHSPTFCHNLVASDLANWNKPSTVKMFHYIDDLMLTSDSIEALEKTVPSLITYLQEKGWAINPQKVQGPGLSVKFLGVVWSGKTKVLPSAIIDKIQAFPVPTKPKQLQEFLGILGYWRSFIPHLAQLLKPLYRLTKKGQVWDWGRTEQEAFQQAKIAVKQAQALGIFDPTLPAELDVHVTQEGFGWGLWQRQGSVRIPIGFWSQIWHGAEERYSMVEKQLLATYSALQAVEPITQTAEVIVKTTLPIQGWVKDLTHIPKTGVAQSQTVARWVAYLSQRSRLSSSPLKEELQKILGPVTYHSETPEEIVVTCPEESPVQEGKYPIPEDAWYTDGSSRGNPSRWRAVAYHPSTETIWFEEGDGQSSQWAELRAVWMVITQEPGNSALNICTDSWAVYRGLTLWIAQWATQDWTIHARPIWGKDMWVDIWNVVRHRTVRAYHVSGHQPLQSPGNDEADTLARV

SEQ ID NO: 56 - protein, 762 aa, synthetic construct
MIDGLGFANSFQNPSHNSLIFISTYVNKRIMNIMVDTGSQHSFINKNCLREFDKLQSFEISPQNFFMADGMTTFQVKDTIRLNISIGTFDTTAVAFVTTHLCTDMILGMDYLSQYDIEIKTKQNYITFNFGNEQVILSTNLESSSKRHSSSNPELIRSSINQQFHTSALSSINNLEQIISNLANHMTNPTQQNNLKSLLFKFQSTFDTSKYTIAKTEIFHVIETETHIPPASKCYPGNPTLNAELRKIVDKLLDAGLIANSQSPYAAPALLVKKKDNTWRLVIDYKKLNSITLKDNYPLPNMEMALQTLGCGYSYFSKFDLRSGFWQLPIDPKDRFKTAFATPFGLFEWLVLPQGLRNSPPTFQRTMNRVLSACTEFSLVYLDDIVIFSHTFDDHLVHIEKVLSALREHNLTLAPSKCELAKQSIEYLGHVISSKTIAPLPDRIKSIILLPEPKTLTQANKFIGSLSWYRKFIPAFATLAAPIHSITNLSKSNRHKFHWGIDQSKSFAALKQFLTSSPIFLDFPVDGQPIFLATDASLVGLGGVLYQEVQGEKRILYYHSELLTPAQKRYHPLELEALAIFKCVNRMKSFLLGREIFVYTDNCPICHMLDKKVSNKRVEKISILLQEFNIRRIIHVKGEYNCLPDYLSRHPIEQDNEFLNSNYGLEFVRDDSSSIQIAGAVVTRSKAKALPPPPTNSSQTNSQQTTSLISPTSNRDSLNELEPEPESVDMNFDLTQIKQHQLGDVRIQQVIEDLKTNPNSSF

SEQ ID NO: 57 - protein, 427 aa, synthetic construct; identical to SEQ ID NO: 22

SEQ ID NO: 58 - protein, 427 aa, synthetic construct; identical to SEQ ID NO: 24

SEQ ID NO: 59 - protein, 427 aa, synthetic construct
MSELLEKILSKDNMNAAYKRVCANKGAGGVDDVTVEELGAYVKENWESIREQIRRRKYTPQPVRRVEIPKPDGGERKLGIPTVMDRVIQQGIVQIISPMCEPLFSKWSYGFRPNRSCEMAIRQLLEYLNEGYEWIVDIDLEKFFDNVPQDRLMSLVHDIIKDGDTESLIRKYLKAGVMTPQGYKETKLGTPQGGNLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIGKINQVTRGWINYYALGSMKTAMTEIDAHLRTRLRVIIWKQWKVPKKRQWGLEKLGVSKELARRTAYCGDSYYWVATKTCVVRAISKEVFAKSGLVSCLDYYNERHALKLC

SEQ ID NO: 60 - protein, 427 aa, synthetic construct
MSELLEKILSKDNMNAAYKRVCANKGAGGIDDVTIEELGDYIKENWESIREQIRHREYKPQPVRRVEIPKPNGGVRELGIPTVMDRVIQQGIVQIISPMCEPLFSEWSYGFRPNRSCEMAVRQLLVYLNEGYEWIVDIDLEKFFDNVPQDRLMSLVHDIIKDGDTESLIRKYLKAGAMTPQGYEETNLGTPQGGNLSPLLSNIILNELDKELEGRGLRFTRYADDCVIAVRSESSAKRVMRTVADWIQRKLGLKVNMTKTRIARPQKLKYLGFGFYKDSKAKEWKCRPHQESVKKLKDRLKELTCRKKPGTVTSKIAEINQVTRGWINYYALGSMKTAMTEIDAHLRTRLRIIIWKQWKVPKKRQWGLQKLGIGKDTARLTAYCGDRYYWVATKTCVVRAISKDVFAKAGLVSCLDYYNERHALKLC

SEQ ID NO: 61 - protein, 427 aa, synthetic construct
MSELLEKILSKENMNAAYKRVCANKGAGGVDGVTVEELGDYIKEHWRSIREQIRQRQYKPQPVRRVEIPKQNGGVRKLGIPTVMDRVIQQGIVQVINPMCEPLFSEWSYGFRPNRSCEMAIRQLLIYLNEGYEWIVDIDLEKFFDNVPQDRLMSLVHVIINDGDTESLIRKYLKAGVMTPQGYEETRLGTPQGGNLSPLLSNIMLNELDKELEARGLRFTRYADDCVIALKSESSAKRVMRTVSDWIQRKLGLKVNMTKTHITKPLKLKYLGFGFYKDSKTKEWKCRAHQDSIVKLKRKLKELTCRKMPGTVREKIEKINQVTRGWINYYALGSMKTAMTAIDAHLRTRLRVIIWKQWKVPKRRQWGLQKLGVGKDLARLTSYCGDRYQWVVTKTCVVRAISKEVLTKAGLISCLEYYNKRHALKLC

SEQ ID NO: 62 - protein, 427 aa, synthetic construct
MSELLEKILSKENMNTAYKRVCANKGAGGVDGVTVEELGDYIKEHWRSIGEQIRQRQYKPQPVRRVEIPKQNGGVRKLGIPTVMDRVIQQGIVQVINPMCEPLFSEWSYGFRPNRSCEMAIRQLLIYLNEGYEWIVDIDLEKFFDNVPQDRLMSLVHVIINDGDTESLIRKYLKAGVMTPQGYEETRLGTPQGGNLSPLLSNIMLNELDKELEARGLRFTRYADDCVIAVKSETSAKRVMRTVSDWIQRKLGLKVNMTKTHITRPLELKYLGFGFYKDSKTKEWKCRAHQDSIVKIKRRLKELTCRKTPGKVREKIEKINQVTRGWINYYALGSMKTAMTAIDAHLRTRLRVIIWKRWKVPKKRQWGLQKLGIGKDLARLTSYCGDRYQWVVTKTCVVRAISEEVLAKAGLISCLEYYNKRHALKLC

SEQ ID NO: 63 - protein, 427 aa, synthetic construct
MSELLDKILHKDNMNEAYKKVCANKGAGGVDGVSTEELGEYIKTNWPTIKEQILRREYKPQPVRRVEIPKPNGGKRKLGIPTAMDRVIQQAIVQVIGPICDPHFSEFSYGFRPNRSCEMAIRKVLEYLNEDYTWIVDIDLEKFFDNVPQDRLLSYVHILINDGDTESLIRKYLKVGVMTPEGYEHTEMGTPQGGNLSPLLSNIMLNELDKELENRGLHFARYADDCIIAVKSEASAKRVMNSITSWIERKLGLKVNAEKTKIVRPTKVKYLGFGFYKDTKTLEWKCKPHQDSVAKFKRRLKALTQRKNSMRLGIRIKKINQVTRGWINYFALGSMKMAISDIDAHLRTRLRTIIWKQWKVPSKRQWGLQKLGVSKDLARLTSYCGDRYQWVVTKTCVKRAISKEVLGRAGLISCLDYYNERHALKLN

SEQ ID NO: 64 - protein, 427 aa, synthetic construct
MSELLDKILDRENMNEAYKRVCANKGAGGVDGVSTEELGDYIRKNWPEIKEQIKRREYKPQPVRRVEIPKPSGGKRKLGIPTAMDRVIQQAIVQAIGPICDPHFSEFSYGFRPKRSCEMAIRKVLEYLNEDYTWIVDIDLEKFFDNVPQDRLMSYLHILINDGDTESLICKYLKAGVMTPEGYERTEMGTPQGGNLSPLLSNIMLNELDKELEHRGLHFARYADDCIIAVKSEASAKRVMYSITSWIERKLGLKVNAEKTKIVRPTNLKYLGFGFYKDYKADEWKCKPHQEAIAKFKRKLKALTQRKSSMKLGLRIEKINQVTRGWINYFALGSMKMALADIDAHLRTRLRTIIWKQWKVPSKRQWGLQKLGVNKDLARLTSYRGNRYQWVVTKTCVVRAISKEVLGKAGLISCLDYYTERHALKLS

SEQ ID NO: 65 - protein, 427 aa, synthetic construct
MSELLEKILDKRNMNEAYKKVCANKGAGGVDGMEIDELDGYIRGNWESIKEQIRKRSYTPQPVRRVEIPKSNGSKRKLGIPTVMDRVIQQGIAQVISPMCEPLFSGNSYGFRPNRSCEMAIREMLVFLNEGYEWIVDIDLEKFFDNVPQDRLMSLVHNIINDGDTESLIRKYLKAGVMIQGRYEKTDRGTPQGGNLSPLLSNIMLNELDKELEKRGLRFVRYADDCVIAVRSEASAKRVMYSITDWIERKLGLKVNAEKTHITRPGKLKYLGFGFYKDPKAKEWKSRPHGESVAKFRRTLKKLTNRSQSMAFAERVQKLNRVIRGWINYFALGSMKTAMNDIDAHLRTRLRVIIWKQWKVPSKRQWGLQKLGIGEDLARVTAYCGNRYQWIVTKTCVVRAISKEKLSQAGLVSCYDYYMERHALKLC

SEQ ID NO: 66 - protein, 427 aa, synthetic construct
MSELLDRILSRDNMNLAYKRVKANKGAGGIDDVEVDELYSYIKENWKSIEGQIRQRTYKPQPVRRVEIPKPNGGKRELGIPTVMDRVIQQAIVQVISPMCEPYFSDRSYGFRPNRSCETAIRKVLEYLNDGYTWIVDIDLEKFFDNVPQDRLMSLVHRIIDDGDTESLIRKYLKAGVMVRGKYERTELGTPQGGNLSPLLSNIMLNELDRELEKRGLNFTRYADDCIIAVRSEASAKRVMHRITEWIERKLGLKVNATKTKITRPSGLKYLGFGFYRDPKADEWKCKPHKDSIAKFKHKLKQLTERRWSINFRVRIAKINQVTRGWINYYALGSMKTAMAEIDAHLRTRLRMIIWKQWKVPAKRQWGLQKLGIGKDLARVTAYCGNRYYWVVTKTCVVRAISKEKLALAGIVSLSDYYAERHMLKLI

SEQ ID NO: 67 - protein, 426 aa, synthetic construct
MSEMLERILDDENIKTAYKRVYANKGAGGVDGVTTEELEEYMRVNWRSIKEQIRERKYNPQAVKRVEIPKPNGGIRKLGIPTVIDRVIEQAITQILTPIFDPLFSDSSYGFRPNRRCEQAIIKLLEHFNDGYVWIVDIDLEKFFDNVPQDKLMSYVGRVIHDGDTESLIRKYLKAGVMVNGRYEATNLGTPQGGNLSPLLSNIMLDELDKELENRGLHFTRYADDCVIVLKSKAAATRVMYSITDWIERKLGLKVNATKTKITPPNKLKYLGFGFWKDKQEWKARPHEDSVEKLKRKLKALCKRNWSIDLTTRIKKINEVTRGWINYFRIASMKSKLQNIDEHLRTQLRVIIWKQWKVPKKREWGLAKLGVGKDLARLTAYCGDRYQWVVTKTCVVRAISKEKLTKKGLVSCLDYYAERRHILNLN

SEQ ID NO: 68 - protein, 412 aa, synthetic construct
MSELLEKILDRDNMNAAYKRVCANKGAGGIDGVTVEELSDYIKENWGSIREQIRQRKYKPQPVRRVEIPKLNGGVRKLGIPTVMDRVIQQGIVQVVSPMCEPMFSERSYGFRPNRSCEMAIRQLLVYLNEGYEWIVDIDLEKFFDNVPQDRLMSLVHDIVKDGDTESLIRKYLKAGVMTRQGYEETNLGTPQGGNLSPLLSNIMLDKLDKELEARGLRFARYADDCVIAVKSEASAKRVMHTVTDWIQRKLGLKVNMTKTHITRPMKLKYLGFGFYKDNKTEEWKCRPHKDSIVRLKSKLKELTCRRMPGKVSEKIKRINQVTRGWINYYALGSMKTVMAEVDAHLRTRLRMNIWKQWKVPKKRQWGLQKLGVDKDSARLTAYYGNHYYKVVKGTCVKRAISKEVLARAGLI

SEQ ID NO: 69 - protein, 424 aa, synthetic construct
MLEKILSKDNMNKAYKAVAANKGSSGVDNVTIEELGSFIKEYWIEIREQIRQRQYKPQPVRRVEIPKPNGGIRKLGIPTVMDRVIQQAIVQVISPICEPYFSDRSYGFRPGRSCEMAIVKLLEYLNDGYEWIVDIDLEKFFDNVPQDKLMSYVHNIIDDGDTESLIRKYLKAGVMTPEGYEETRLGTPQGGNLSPLLSNIMLNELDKELEARGLNFARYADDCVIAAKTDMSARRIMRNITSWIERKLGLKVNATKTKVVRPTKLKYLGFGFYKDIQTKKWMSRPHEESVEKFKRKLKQLTCRSTSMKFKIRINKLNQVIRGWINYFSLGSMKTKMKEIDEHLRTRLRVVLWKQWKKPSKRQWALQKLGIGSDLARQTAYMGDHYQWVVTKTCVVRAISKENLGQFGLVSCLDYYTERHSLKFN

SEQ ID NO: 70 - protein, 439 aa, synthetic construct
MLQLLEEVLDRNNMNLAYEKVYANKGAGGVDGVTLDELKAYIKENWPDVQTQIRNRTYQPKPVKRVEIPKANGGTRNLGIPTVMDRVIQQAMVQVLSPICEPHFSEYSYGFRPKRSCEMAILKLLDYFNDGFLWIVDIDLEKFFDNVPQDRLMSYVHNIINDGDTESLIRKYLKAGVMINGVKHKTEVGTPQGGNLSPLLSNIMLTELDKELEARGLKFTRYADDCIIVVKSEASAKRVMRTITDWIERKLGLKVNMTKTQVTGPTKLKYLGFGFYFDYKSKTWKTRPHEDSVTKFKRKLKQLTKRNWSINLRERIKKINGVIRGWINYFSLCSMKTHMEKIDRHLRTRIRVIVWKQWKVPSKRQWGLQKLGIGRDLAKQTSYMGNHYQWVVTKTCVVRAISKEKLAQAGLVHCLDYYLNQHTLKFNRTAVCRTACTVV

SEQ ID NO: 71 - protein, 449 aa, synthetic construct
MSELLEKILNRENMNKAYKRVKANKGTSGIDEITIEDAYVYIKENWESIRAEITERKYKPQPVKRVEIPKPNGGTRNLGIPTVMDRIIQQAMVQVLSPICETFFSDYSYGFRPNRSCEQAINKLLEYINDGYEWIVDIDLEKFFDNVPQDKLMSYVHIIINDGDTESLIRKYLKAGIMINGKYEKSEKGTPQGGNLSPLLSNIILNELDKELESRGLHFTRYADDCVIAVKSRASANRVMHTITKWIEHKLGLKVNATAAAGLKVNATKTHITRPNKLKYLGFGFYYDTKAEKYCARPHASSIQRFKRKLKQLTIRKNTMALKERIRRLNQVIRGWINYYSICNMKTHMTRIDEHLRTRLRVIIWKQWKVPSKRQWGLQKLGISKDRARQTSYMGDHYQWVVTKTCVVRAISKEKLAQKGLVSCLDYYIERHALKLKRTAVYGTVRTVV

SEQ ID NO: 72 - protein, 439 aa, synthetic construct
MSELLEKILNRDNMNQAYKRVKANKGTSGIDEVTVNEAYGYIEENWERIKQEITERRYKPQPVKRVEIPKPNGGTRNLGIPTVMDRIIQQAMVQVLSPMCEEIFSEYSYGFRPGRSCEQAIIKVLEYFNDGYGWIVDIDLEKFFDNVPQDKLMSYVHTIINDGDTESLIRKFLKAGIMINGRYEESEKGTPQGGNLSPLLSNIILSELDKELESRGLKFVRYADDCVIAVKSRASANRVMHTITSWIEGKLGLKVNATKTQITKPNKLKYLGFGFYYHPKEKKYCARPHESSIQQFKRKLKRLTIRKNTMTLEVRIKQLNQVIRGWINYYAIGNMKTHMARIDSHLRTRLRVIIWKQWKVPSKRQWGLQKLGINKDRARQTSYMGDHYQWVVTKTCVVRAISKERLTQKGLVSCLDYYMDRHNLKLERTAVYGTVRTVV

SEQ ID NO: 73 - protein, 453 aa, synthetic construct
MRNVNEQILHYLGRIHENGKEKAHKAMSELLEKILSKDNMNAAYKRVYANKGAGGVDDVTVGELGGYIKENWESIREQIRRREYKPQPVRRVEIPKPNGGVRELGIPTVMDRVIQQGIVQVISPVCEPLFSECSYGFRPNRSCEMAVRQLLIYLNEGYEWIVDIDLERFFDNVPQDKLMSLVHNIINDGDTESLIRKYLKAGVMTPDGYEETKLGTPQGGNLSPLLSNIMLNELDKELEARGLRFTRYADDCVIAVRSESSAKRVMRTVSDWIQRKLGLKVNTTKTHITRPQKLKYLGFGFYKDSKAKEWKCRPHQESVKKLKRRLKELTCRKMPGTVTGRIAKINQVTRGWINYYALGSMKTAMTEIDAHLRTRLRIIIWKQWKVPKKRQWGLQKLGVDKDLARLTAYCGDRYYWVATKTCVVRAISKDVFAKAGLISCLDYYNERHALKLC

SEQ ID NO: 74 - protein, 427 aa, synthetic construct
MSELLEKILSKENMNTAYKRVCANKGAGGVDGVTVEELGDYIKEHWRSIREQIRQRQYKPQPVRRVEIPKQNGGVRELGIPTVMDRVIQQGIVQVINPMCEPLFSEWSYGFRPNRSCEMAIRQLLIYLNEGYEWIVDIDLEKFFDNVPQDRLMSLVHDIINDGDTESLIRKYLKAGVMTPQGYEETRLGTPQGGNLSPLLSNIMLNELDKELEARGLRFTRYADDCVIAVKSESSAKRVMRTVSDWIQRKLGLKVNMTKTHITRPLKLKYLGFGFYKDSKTKEWKCRAHQDSIVKLKRKLKELTCRKTPGKVREKIEKINQVTRGWINYYALGSMKTAMTAIDAHLRTRLRVIIWKQWKVPKKRQWGLQKLGVGKDLARLTSYCGDRYQWVVTKTCVVRAISKEVLAKAGLISCLEYYNKRHALKLC

SEQ ID NO: 75 - protein, 427 aa, synthetic construct
MSELLEKILSKNNMNAAYKKVCANKGAGGVDNVTVEELGDYIKENWESIREQIRRREYKPQPVRRVEIPKPDGGVRKLGIPTVMDRVIQQGIVQVISPMCEPVFSEWSYGFRPNRSCEMAVRQLLVYLNEGYEWIVDIDLERFFDNVPQDRLMSLVHDIINDGDTESLIRKYLKAGVMSRQGYEETKLGTPQGGNLSPLLSNIMLNELDKELEARGLRFTRYADDCVIVVRSESSAKRVMRTVADWIQRKLGLKVNMTKTHITRPQKLKYLGFGFYRDSRAKEWKCRPHQDSVKKFKRKLKELTCRKMPGTVTGRIVQINQVTRGWINYYALGSMKTAMTEIDAHLRTRLRIIIWKQWKVPKKRQWGLQKLGIGKDLARLTAYCGDRYYWVATKTCVVRAISKDVFARSGLISCLDYYNERHALKLC

SEQ ID NO: 76 - protein, 427 aa, synthetic construct
MSELLEKILSKKNMNAAYKKICANKGAGGVDDVTVEELGDYIKENWESIREQIRRREYKPQPVRRVEIPKPDGGVRKLGIPTVMDRVIQQGIVQIISPMCEPLFSEWSYGFRPNRSCEMAVRQLLMYLNEGYEWIVDIDLEKFFDNVPQDRLMSLVHDIINDGDTESLIRKYLKAGVMTPQGYKETKLGTPQGGNLSPLLSNIMLNELDKELEARGLRFTRYADDCVIAVRSESSAKRVMRTVTDWIQRKLGLKVNMTKTHITRPQKLKYLGFGFYKDSKAKEWKCRPHQDSVKKFKRKLKELTCRKMPGTVTGKIVKINQVTRGWVNYYALGSMKTAMTEIDAHLRTRLRIIIWKQWKAPKKRQWGLQKLGIDKDLARLTAYCGDRYYWVATKTCVNRAISKKVLTKAGLVSCLDYYNERHALKLC

SEQ ID NO: 77 - protein, 427 aa, synthetic construct
MSELLEKILSQNNMNAAYKKVCANKGAGGVDNVTVEELGDYIKENWESIREQIRRREYKPQPVRRVEIPKPDGGVRKLGIPTVMDRVIQQGIVQVISPMCEPMFSEWSYGFRPNRSCEMAIRQLLVYLNEGYEWIVDIDLERFFDNVPQDRLMSLVHDIINDGDTESLIRKYLKAGVMSRQGYEETKLGTPQGGNLSPLLSNIMLNELDKELGARGLRFTRYADDCVIAVSSESSAKRVMRTVADWIQRKLGLKVNMTKTHITRPQKLKYLGFGFYKDSREKQWKCRPHQDSVKKFKRKLKELTCRKMPGTVTGKIVQINQVTRGWINYYALGSMKTAMTEIDAHLRTRLRIIIWKQWKVPKKRQWGLQKLGIGKDLARLTAYCGDRYYWVATKTCVVRAISKDVFAKAGLISCLDYYNEHHALKLC

SEQ ID NO: 78 - protein, 449 aa, synthetic construct
MSRYCNHLGQIHENGKEKAHKAMSELLEKVLSKDNMNAAYKRVCANKGAGGVDDVTVEELGDYIKENWEGIREQIRQREYRPQPVRRVEIPKPNGGVRKLGIPTVMDRVIQQGIVQIISPMCEPLFSERSYGFRPDRSCEMAVRQLLVYLNEGYEWIVDIDLEKFFDNVPQDRLMSLVHDIIKDGDTESLIRKYLKAGVMTPQGYEETNLGTPQGGNLSPLLSNIMLNELDKELEERGLRFTRYADDCVIAVKSEASAKRVMRTVTGWIQRKLGLKVNMTKTRITRPQRLKYLGFGLYKDSKAKEWKCRPHQESVKKFKGRLKELTCRKMPGTVTGRIAEINQVTRGWINYYALGSMKTAMTETDAHLRTRLRIIIWKQWKVPKKRQWGLQKLGVSKDLARLTAYCGDRYYWVATKTCVVRAISKDVFAKAGLVSCLDYYNERHALKLC

SEQ ID NO: 79 - protein, 427 aa, synthetic construct
MSELLEKILSKNNMNAAYKKICANKGAGGVDDVTVEELGDYIKENWESIREQIRRREYKPQPVRRVEIPKSDGGVRKLGIPTVMDRVIQQGIVQIISPMCEPLFSEWSYGFRPNRSCEMAVRQLLMYLNEGYEWIVDIDLEKFFDNVPQDRLMSLVHDIINDGDTESLIRKYLKAGVMTPQGYKETKFGTPQGGNLSPLLSNIMLNELDKELEARGLRFTRYADDCVIAVRSESSAKRVMRTVTDWIQRKLGLKVNMTKTHITRPQKLKYLGFGFYKDSKAKEWKCRPHQDSVKKFKRKLKELTCRKMPGTVTGKIVKINQVTRGWVNYYALGSMKTAMTEIDAHLRTRLRIIIWKQWKAPKKRQWGLQKLGIDKDLARLTAYCGDRYYWVATKTCVNRAISKKVLTKAGLVSCLDYYNERHALKLC

SEQ ID NO: 80 - protein, 427 aa, synthetic construct
MSELLEKILSKRNMNTAYKRVCANKGAGGVDDVTVEELGAYVKENWESIREQIRRREYTPQPVRRVEIPKPRGGERKLGIPTVMDRVIQQGIVQIISPMCEPLFSKWSYGFRPNRSCEMAIRQLLEYLNEGYEWIVDIDLEKFFDNVPQDRLMSLVHDIIKDGDTESLIRKYLKAGAMTPQGYKETKLGTPQGGRLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 81 - protein, 427 aa, synthetic construct
MSELLEKILSKRNMNTAYKRVCARKGAGGVDDVTVEELGAYVKENWESIREQIRRREYTPQPVRRVEIPKPRGGERKLGIPTVMDRVIQQGIVQIISPMCEPLFSKWSYGFRPKRSCEMAIRQLLEYLNEGYEWIVDIDLEKFFDNVPQDRLMSLVHDIIKDGDTESLIRKYLKAGAMTPQGYKETKLGTPQGGNLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 82 - protein, 427 aa, synthetic construct
MSELLEKILSKRNMNTAYKRVCARKGAGGVDDVTVEELGAYVKENWESIREQIRRREYTPQPVRRVEIPKPRGGERKLGIPTVMDRVIQQGIVQIISPMCEPLFSKWSYGFRPNRSCEMAIRQLLEYLNEGYEWIVDIDLEKFFDNVPQDRLMSLVHDIIKDGDTESLIRKYLKAGAMTPQGYKETKLGTPQGGRLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 83 - protein, 427 aa, synthetic construct
MSELLEKILSKRNMNTAYKRVCANKGAGGVDDVTVEELGAYVKENWESIREQIRRREYTPQPVRRVEIPKPRGGERKLGIPTVMDRVIQQGIVQIISPMCEPLFSKWSYGFRPKRSCEMAIRQLLEYLNEGYEWIVDIDLEKFFDNVPQDRLMSLVHDIIKDGDTESLIRKYLKAGAMTPQGYKETKLGTPQGGRLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 84 - protein, 427 aa, synthetic construct
MSELLEKILSKRNMNTAYKRVCARKGAGGVDDVTVEELGAYVKENWESIREQIRRREYTPQPVRRVEIPKPRGGERKLGIPTVMDRVIQQGIVQIISPMCEPLFSKWSYGFRPKRSCEMAIRQLLEYLNEGYEWIVDIDLEKFFDNVPQDRLMSLVHDIIKDGDTESLIRKYLKAGAMTPQGYKETKLGTPQGGRLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 85 - protein, 427 aa, synthetic construct
MSELLERILSNRNMNAAYKRVCANKGAGGVDGVTVEELGDYIKENWSSIREQIRRRQYKPQPVRRVEIPKPRGGVRNLGIPTVMDRVIQQGIVQVLSPMCEPLFSERSYGFRPNRSCEMAIRQLLVYLNEGCEWIVDIDLEKFFDNVPQDRLMSLVHDIINDGDTESLIRKYLKAGVMTPQGYEETKLGTPQGGRLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 86 - protein, 427 aa, synthetic construct
MSELLERILSNRNMNAAYKRVCARKGAGGVDGVTVEELGDYIKENWSSIREQIRRRQYKPQPVRRVEIPKPRGGVRNLGIPTVMDRVIQQGIVQVLSPMCEPLFSERSYGFRPKRSCEMAIRQLLVYLNEGCEWIVDIDLEKFFDNVPQDRLMSLVHDIINDGDTESLIRKYLKAGVMTPQGYEETKLGTPQGGNLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 87 - protein, 427 aa, synthetic construct
MSELLERILSNRNMNAAYKRVCARKGAGGVDGVTVEELGDYIKENWSSIREQIRRRQYKPQPVRRVEIPKPRGGVRNLGIPTVMDRVIQQGIVQVLSPMCEPLFSERSYGFRPNRSCEMAIRQLLVYLNEGCEWIVDIDLEKFFDNVPQDRLMSLVHDIINDGDTESLIRKYLKAGVMTPQGYEETKLGTPQGGRLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 88 - protein, 427 aa, synthetic construct
MSELLERILSNRNMNAAYKRVCANKGAGGVDGVTVEELGDYIKENWSSIREQIRRRQYKPQPVRRVEIPKPRGGVRNLGIPTVMDRVIQQGIVQVLSPMCEPLFSERSYGFRPKRSCEMAIRQLLVYLNEGCEWIVDIDLEKFFDNVPQDRLMSLVHDIINDGDTESLIRKYLKAGVMTPQGYEETKLGTPQGGRLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 89 - protein, 427 aa, synthetic construct
MSELLERILSNRNMNAAYKRVCARKGAGGVDGVTVEELGDYIKENWSSIREQIRRRQYKPQPVRRVEIPKPRGGVRNLGIPTVMDRVIQQGIVQVLSPMCEPLFSERSYGFRPKRSCEMAIRQLLVYLNEGCEWIVDIDLEKFFDNVPQDRLMSLVHDIINDGDTESLIRKYLKAGVMTPQGYEETKLGTPQGGRLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 90 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 91 - protein, 1844 aa, synthetic construct
MSELLERILSNDNMNAAYKRVCANKGAGGVDGVTVEELGDYIKENWSSIREQIRRRQYKPQPVRRVEIPKPNGGVRNLGIPTVMDRVIQQGIVQVLSPMCEPLFSERSYGFRPNRSCEMAIRQLLVYLNEGCEWIVDIDLEKFFDNVPQDRLMSLVHDIINDGDTESLIRKYLKAGVMTPQGYEETKLGTPQGGNLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLCGSSGGSPAGSPTSTEEGTSESATPESGPGTSTEPSEGSAPGSPAGSGGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 92 - protein, 1842 aa, synthetic construct
MLDRDNLRLAYKRVVRNGGAPGVDGVTVAALQSYLNTHWDRVKNELLAGTYRPAPVRRVEIPKPGGGVRLLGIPTVMDRFLQQALLQVMNPIFDAHFSWYSYGFRPGKRAHDAVMQAQRYIRDGYRWVVDLDLEKFFDRVNHDMLMARVARKVKDKRVLKLIRAYLNAGVMINGVCQRTEEGTPQGGPLSPLLANILLDDLDKELTRRGLRFVRYADDCNIFVASKRAGERVMESAIRFLEGKLKLKVNRDKSAVDRPWKRKFLGFSFLSDKEATIRLAPKTISRFKERVREITSRSRPIPMEERIRRLNQYTMGWVSYFRLASMKNHCERFDQWIRRRLRMCLWKQWKRVRTRIRELRALGVPDWACFAMANSRRGPWEMSRNINNALPTSYWEAKGLKSMLTRYMVLRQPFGTAWCGPACQVVGSSGGSPAGSPTSTEEGTSESATPESGPGTSTEPSEGSAPGSPAGSGGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 93 - protein, 2178 aa, synthetic construct
ENPIKIDFFEPLEIEPLNLIDVNFVEKMDMDIDLSYEQDSLLKQNLKNLRLDHCNKEEKDAIRKLCFDYRDIFYCEQIPLSFTSEITHKIKLNDESPIYTKSYRFPEIHKKEVKDQIAKMLDQGIIQHSISPWSSPIWVVPKKLDASGRRKWRLVIDYRKLNEKSCNDRYPLPNITDILDKLGRANYFTTLDLASGFHQIQVDPDDIPKTAFSTEGGHYEFKRMPFGLKNAPSTFQRVMDNILRGLHNEICLVYLDDIIIFSTSLDEHIQRLKSVFDRLRKSNFKLQLDKSEFLQKTVQYLGHIITPQGVKPNPDKVSTIKRFPIPRTQKDIKSFLGLLGYYRRFIKDFAKITKPLTKCLKKNAKVTHDQNFIDAFNTCKEILVNDPILQYPDFSKPFILTTDASDVALGAILSQGTLPNDRPVAYASRTLNETESKYSTIEKELLAIVWACKHFRPYLYGRKFTIYTDHRPLTWLFSLKEPNSKLVRWRLKLEEFEYDIIYKKGKLNTNADCLSRVTLNAIDNESMLNNPGDIDQDILDTLQNVTQNLQTLQDFQKPSTSNTNDKINIISDIQIKPPDNSQNDNTNTSTQHSTANETTNDGIKIFDEIINNKTNQILVFPNVIYKLDIKRETYENHKIVTVKIPIINNEQMILQFLKENTDPKHVYCMYFQSDELYNDFCTVYLKHFSEKGPKLIRCLKLVNTVADKEEQILLIKNHHGSKTNHRGINETLEKLKFNYYWKNMKSTVSNFINACDICQRAGSSGGSPAGSPTSTEEGTSESATPESGPGTSTEPSEGSAPGSPAGSGGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 94 - protein, 2072 aa, synthetic construct
VLIRDTSITTNPLPARYHDFSDVFEKKNADRLPEHRLYDCPIDLQEGAHPPFGPIYGLAEPELEALREYLKENLAKGFIQPSKSPAGAPILFVKKKDGSLRLCVDYRGLNRVTVRNRYPLPLIPELLDRLRTGRVFSKIDLRGAYNLVRIKPGDEWKTAFRTRYGHFEYKVMPFGLTNAPAVFQHMMNHIFREYLDHFVVIYLDDILVFSPSMEEHTRHVRLILTKLREHGLYAKSEKCEFDRTSVEFLGYVISPAGITMDPRKVATIHNWPIPTRLKEVQSFLGFANFYRRFIDRFSTIVQPLISLTRKDVPFVWTATTQHAFDALKQAFMSAPVLVHPNPAKPFQVETDASDFALGAILSQLDDDGTLHPVAYYSRKFLASEINYPVYDKELAAIISAFAEWRPYLVGAQHRIQVVTDHKNLLYFASSRTLNRRQARWSIFLADYDFEIIFRPGIQHGKADALSRRPDLALCPGDDAYTQQSCSLLKPDQLQLSATFMLHDDSLLQEIAQATKTDTFATEILKRLQDSSPATKRADLHHFTTHDGLLYRNHLLYVPEGPCRTQVLQTCHDDPLAGHFGVAKTLELVSRGFWWPQPWKLVKEFVKTCDVCARSKAVHHRPYGLLHPLPIPNRPWASISMDFITDLPPVNGVDTVGSSGGSPAGSPTSTEEGTSESATPESGPGTSTEPSEGSAPGSPAGSGGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 95 - protein, 1844 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSSGGSPAGSPTSTEEGTSESATPESGPGTSTEPSEGSAPGSPAGSGGGSMSELLEKILSKDNMNTAYKRVCANKGAGGVDDVTVEELGAYVKENWESIREQIRRREYTPQPVRRVEIPKPDGGERKLGIPTVMDRVIQQGIVQIISPMCEPLFSKWSYGFRPNRSCEMAIRQLLEYLNEGYEWIVDIDLEKFFDNVPQDRLMSLVHDIIKDGDTESLIRKYLKAGAMTPQGYKETKLGTPQGGNLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 96 - protein, 1844 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSSGGSPAGSPTSTEEGTSESATPESGPGTSTEPSEGSAPGSPAGSGGGSMSELLERILSNDNMNAAYKRVCANKGAGGVDGVTVEELGDYIKENWSSIREQIRRRQYKPQPVRRVEIPKPNGGVRNLGIPTVMDRVIQQGIVQVLSPMCEPLFSERSYGFRPNRSCEMAIRQLLVYLNEGCEWIVDIDLEKFFDNVPQDRLMSLVHDIINDGDTESLIRKYLKAGVMTPQGYEETKLGTPQGGNLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 97 - protein, 1842 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSSGGSPAGSPTSTEEGTSESATPESGPGTSTEPSEGSAPGSPAGSGGGSMLDRDNLRLAYKRVVRNGGAPGVDGVTVAALQSYLNTHWDRVKNELLAGTYRPAPVRRVEIPKPGGGVRLLGIPTVMDRFLQQALLQVMNPIFDAHFSWYSYGFRPGKRAHDAVMQAQRYIRDGYRWVVDLDLEKFFDRVNHDMLMARVARKVKDKRVLKLIRAYLNAGVMINGVCQRTEEGTPQGGPLSPLLANILLDDLDKELTRRGLRFVRYADDCNIFVASKRAGERVMESAIRFLEGKLKLKVNRDKSAVDRPWKRKFLGFSFLSDKEATIRLAPKTISRFKERVREITSRSRPIPMEERIRRLNQYTMGWVSYFRLASMKNHCERFDQWIRRRLRMCLWKQWKRVRTRIRELRALGVPDWACFAMANSRRGPWEMSRNINNALPTSYWEAKGLKSMLTRYMVLRQPFGTAWCGPACQVV

SEQ ID NO: 98 - protein, 2178 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSSGGSPAGSPTSTEEGTSESATPESGPGTSTEPSEGSAPGSPAGSGGGSENPIKIDFFEPLEIEPLNLIDVNFVEKMDMDIDLSYEQDSLLKQNLKNLRLDHCNKEEKDAIRKLCFDYRDIFYCEQIPLSFTSEITHKIKLNDESPIYTKSYRFPEIHKKEVKDQIAKMLDQGIIQHSISPWSSPIWVVPKKLDASGRRKWRLVIDYRKLNEKSCNDRYPLPNITDILDKLGRANYFTTLDLASGFHQIQVDPDDIPKTAFSTEGGHYEFKRMPFGLKNAPSTFQRVMDNILRGLHNEICLVYLDDIIIFSTSLDEHIQRLKSVFDRLRKSNFKLQLDKSEFLQKTVQYLGHIITPQGVKPNPDKVSTIKRFPIPRTQKDIKSFLGLLGYYRRFIKDFAKITKPLTKCLKKNAKVTHDQNFIDAFNTCKEILVNDPILQYPDFSKPFILTTDASDVALGAILSQGTLPNDRPVAYASRTLNETESKYSTIEKELLAIVWACKHFRPYLYGRKFTIYTDHRPLTWLFSLKEPNSKLVRWRLKLEEFEYDIIYKKGKLNTNADCLSRVTLNAIDNESMLNNPGDIDQDILDTLQNVTQNLQTLQDFQKPSTSNTNDKINIISDIQIKPPDNSQNDNTNTSTQHSTANETTNDGIKIFDEIINNKTNQILVFPNVIYKLDIKRETYENHKIVTVKIPIINNEQMILQFLKENTDPKHVYCMYFQSDELYNDFCTVYLKHFSEKGPKLIRCLKLVNTVADKEEQILLIKNHHGSKTNHRGINETLEKLKFNYYWKNMKSTVSNFINACDICQRA

SEQ ID NO: 99 - protein, 2072 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSSGGSPAGSPTSTEEGTSESATPESGPGTSTEPSEGSAPGSPAGSGGGSVLIRDTSITTNPLPARYHDFSDVFEKKNADRLPEHRLYDCPIDLQEGAHPPFGPIYGLAEPELEALREYLKENLAKGFIQPSKSPAGAPILFVKKKDGSLRLCVDYRGLNRVTVRNRYPLPLIPELLDRLRTGRVFSKIDLRGAYNLVRIKPGDEWKTAFRTRYGHFEYKVMPFGLTNAPAVFQHMMNHIFREYLDHFVVIYLDDILVFSPSMEEHTRHVRLILTKLREHGLYAKSEKCEFDRTSVEFLGYVISPAGITMDPRKVATIHNWPIPTRLKEVQSFLGFANFYRRFIDRFSTIVQPLISLTRKDVPFVWTATTQHAFDALKQAFMSAPVLVHPNPAKPFQVETDASDFALGAILSQLDDDGTLHPVAYYSRKFLASEINYPVYDKELAAIISAFAEWRPYLVGAQHRIQVVTDHKNLLYFASSRTLNRRQARWSIFLADYDFEIIFRPGIQHGKADALSRRPDLALCPGDDAYTQQSCSLLKPDQLQLSATFMLHDDSLLQEIAQATKTDTFATEILKRLQDSSPATKRADLHHFTTHDGLLYRNHLLYVPEGPCRTQVLQTCHDDPLAGHFGVAKTLELVSRGFWWPQPWKLVKEFVKTCDVCARSKAVHHRPYGLLHPLPIPNRPWASISMDFITDLPPVNGVDTV

SEQ ID NO: 100 - RNA, 20 nt, synthetic construct
  source 1..20: mol_type = other RNA
gtttctgtgattttcttttg

SEQ ID NO: 101 - RNA, 20 nt, synthetic construct
  source 1..20: mol_type = other RNA
tgattttcttttggattgca

SEQ ID NO: 102 - RNA, 20 nt, synthetic construct
  source 1..20: mol_type = other RNA
attttcttttggattgcatc

SEQ ID NO: 103 - RNA, 20 nt, synthetic construct
  source 1..20: mol_type = other RNA
caatccaaaagaaaatcaca

SEQ ID NO: 104 - RNA, 20 nt, synthetic construct
  source 1..20: mol_type = other RNA
gatgcaatccaaaagaaaat

SEQ ID NO: 105 - RNA, 20 nt, synthetic construct
  source 1..20: mol_type = other RNA
cagtagatgcaatccaaaag

SEQ ID NO: 106 - RNA, 20 nt, synthetic construct
  source 1..20: mol_type = other RNA
gattgcatctactgtatagg

SEQ ID NO: 107 - RNA, 20 nt, synthetic construct
  source 1..20: mol_type = other RNA
tacagtagatgcaatccaaa

SEQ ID NO: 108 - RNA, 20 nt, synthetic construct
  source 1..20: mol_type = other RNA
catctactgtatagggaccc

SEQ ID NO: 109 - RNA, 20 nt, synthetic construct
  source 1..20: mol_type = other RNA
ctgtatagggaccctccttc

SEQ ID NO: 110 - RNA, 20 nt, synthetic construct
  source 1..20: mol_type = other RNA
tatagggaccctccttccat

SEQ ID NO: 111 - RNA, 20 nt, synthetic construct
  source 1..20: mol_type = other RNA
gaaggagggtccctatacag

SEQ ID NO: 112 - RNA, 20 nt, synthetic construct
  source 1..20: mol_type = other RNA
tagggaccctccttccatga

SEQ ID NO: 113 - RNA, 20 nt, synthetic construct
  source 1..20: mol_type = other RNA
gggaccctccttccatgact

SEQ ID NO: 114 - RNA, 20 nt, synthetic construct
  source 1..20: mol_type = other RNA
agtcatggaaggagggtccc

SEQ ID NO: 115 - RNA, 20 nt, synthetic construct
  source 1..20: mol_type = other RNA
actcaagcttggctctggcc

SEQ ID NO: 116 - RNA, 20 nt, synthetic construct
  source 1..20: mol_type = other RNA
ggacaggccagagccaagct

SEQ ID NO: 117 - RNA, 20 nt, synthetic construct
  source 1..20: mol_type = other RNA
gctctggcctgtcctaagac

SEQ ID NO: 118 - RNA, 20 nt, synthetic construct
  source 1..20: mol_type = other RNA
gcctgtcctaagacctgctc

SEQ ID NO: 119 - RNA, 20 nt, synthetic construct
  source 1..20: mol_type = other RNA
agcaggtcttaggacaggcc

SEQ ID NO: 120 - RNA, 20 nt, synthetic construct
  source 1..20: mol_type = other RNA
tcctaagacctgctcagctt

SEQ ID NO: 121 - RNA, 20 nt, synthetic construct
  source 1..20: mol_type = other RNA
agacctgctcagcttcttcc

SEQ ID NO: 122 - RNA, 20 nt, synthetic construct
  source 1..20: mol_type = other RNA
aggaagaagctgagcaggtc

SEQ ID NO: 123 - RNA, 20 nt, synthetic construct
  source 1..20: mol_type = other RNA
ctcagcttcttccttagctt

SEQ ID NO: 124 - RNA, 20 nt, synthetic construct
  source 1..20: mol_type = other RNA
gaagctaaggaagaagctga

SEQ ID NO: 125 - RNA, 20 nt, synthetic construct
  source 1..20: mol_type = other RNA
gctggaagctaaggaagaag

SEQ ID NO: 126 - RNA, 20 nt, synthetic construct
  source 1..20: mol_type = other RNA
gcttccagccattgtgttga

SEQ ID NO: 127 - RNA, 20 nt, synthetic construct
  source 1..20: mol_type = other RNA
aaggattcaacacaatggct

SEQ ID NO: 128 - RNA, 20 nt, synthetic construct
  source 1..20: mol_type = other RNA
ttaaaggattcaacacaatg

SEQ ID NO: 129 - RNA, 20 nt, synthetic construct
  source 1..20: mol_type = other RNA
tgttgaatcctttaacattt

SEQ ID NO: 130 - RNA, 20 nt, synthetic construct
  source 1..20: mol_type = other RNA
ttgaatcctttaacatttca

SEQ ID NO: 131 - RNA, 20 nt, synthetic construct
  source 1..20: mol_type = other RNA
aaatgttaaaggattcaaca

SEQ ID NO: 132 - RNA, 20 nt, synthetic construct
  source 1..20: mol_type = other RNA
aatcctttaacatttcattc

SEQ ID NO: 133 - RNA, 20 nt, synthetic construct
  source 1..20: mol_type = other RNA
aatgaaatgttaaaggattc

SEQ ID NO: 134 - RNA, 20 nt, synthetic construct
  source 1..20: mol_type = other RNA
acatttcattcaactgttgc

SEQ ID NO: 135 - RNA, 20 nt, synthetic construct
  source 1..20: mol_type = other RNA
ttgcctccggttctgaaggt

SEQ ID NO: 136 - RNA, 20 nt, synthetic construct
  source 1..20: mol_type = other RNA
cctccggttctgaaggtgtt

SEQ ID NO: 137 - RNA, 20 nt, synthetic construct
  source 1..20: mol_type = other RNA
caagaacaccttcagaaccg

SEQ ID NO: 138 - RNA, 20 nt, synthetic construct
  source 1..20: mol_type = other RNA
aaggtgttcttgtacttcat

SEQ ID NO: 139 - RNA, 20 nt, synthetic construct
  source 1..20: mol_type = other RNA
aagtacaagaacaccttcag

SEQ ID NO: 140 - RNA, 20 nt, synthetic construct
  source 1..20: mol_type = other RNA
ttcttgtacttcatcccact

SEQ ID NO: 141 - RNA, 20 nt, synthetic construct
  source 1..20: mol_type = other RNA
ggatgaagtacaagaacacc

SEQ ID NO: 142 - RNA, 20 nt, synthetic construct
  source 1..20: mol_type = other RNA
tacttcatcccactgattct

SEQ ID NO: 143 - RNA, 20 nt, synthetic construct
  source 1..20: mol_type = other RNA
cttcatcccactgattctga

SEQ ID NO: 144 - RNA, 20 nt, synthetic construct
  source 1..20: mol_type = other RNA
gaacaatcattacggatcga

SEQ ID NO: 145 - RNA, 20 nt, synthetic construct
  source 1..20: mol_type = other RNA
atgattgttctagcctcttg

SEQ ID NO: 146 - RNA, 20 nt, synthetic construct
  source 1..20: mol_type = other RNA
attgttctagcctcttgatt

SEQ ID NO: 147 - RNA, 20 nt, synthetic construct
  source 1..20: mol_type = other RNA
ttctagcctcttgattgctg

SEQ ID NO: 148 - RNA, 20 nt, synthetic construct
  source 1..20: mol_type = other RNA
gcctcttgattgctggtctt

SEQ ID NO: 149 - RNA, 20 nt, synthetic construct
  source 1..20: mol_type = other RNA
attgctggtcttgtttttca

SEQ ID NO: 150 - RNA, 20 nt, synthetic construct
  source 1..20: mol_type = other RNA
aaaaacaagaccagcaatca

SEQ ID NO: 151 - RNA, 20 nt, synthetic construct
  source 1..20: mol_type = other RNA
ctggtcttgtttttcaaatt

SEQ ID NO: 152 - RNA, 20 nt, synthetic construct
  source 1..20: mol_type = other RNA
gtcttgtttttcaaattttg

SEQ ID NO: 153 - RNA, 20 nt, synthetic construct
  source 1..20: mol_type = other RNA
tttttcaaattttgggcagc

SEQ ID NO: 154 - RNA, 20 nt, synthetic construct
  source 1..20: mol_type = other RNA
cccaaaatttgaaaaacaag

SEQ ID NO: 155 - RNA, 20 nt, synthetic construct
  source 1..20: mol_type = other RNA
ccgctgcccaaaatttgaaa

SEQ ID NO: 156 - RNA, 20 nt, synthetic construct
  source 1..20: mol_type = other RNA
ggcagcggtaatgagttctt

SEQ ID NO: 157 - RNA, 20 nt, synthetic construct
  source 1..20: mol_type = other RNA
gaagaactcattaccgctgc

SEQ ID NO: 158 - RNA, 20 nt, synthetic construct
  source 1..20: mol_type = other RNA
atgagttcttccaactgggg

SEQ ID NO: 159 - RNA, 20 nt, synthetic construct
  source 1..20: mol_type = other RNA
agttcttccaactggggacg

SEQ ID NO: 160 - RNA, 20 nt, synthetic construct
  source 1..20: mol_type = other RNA
gaacagaggcgtccccagtt

SEQ ID NO: 161 - RNA, 20 nt, synthetic construct
  source 1..20: mol_type = other RNA
gggacgcctctgttccaaat

SEQ ID NO: 162 - RNA, 20 nt, synthetic construct
  source 1..20: mol_type = other RNA
caggatttggaacagaggcg

SEQ ID NO: 163 - RNA, 20 nt, synthetic construct
  source 1..20: mol_type = other RNA
ttccaaatcctgcattgttg

SEQ ID NO: 164 - RNA, 20 nt, synthetic construct
  source 1..20: mol_type = other RNA
cttgatgatcatctcgttga

SEQ ID NO: 165 - RNA, 20 nt, synthetic construct
  source 1..20: mol_type = other RNA
atgatcatctcgttgatatc

SEQ ID NO: 166 - RNA, 20 nt, synthetic construct
  source 1..20: mol_type = other RNA
tcaacgagatgatcatcaag

SEQ ID NO: 167 - RNA, 20 nt, synthetic construct
  source 1..20: mol_type = other RNA
atcatctcgttgatatcctc

SEQ ID NO: 168 - RNA, 20 nt, synthetic construct
  source 1..20: mol_type = other RNA
aggatatcaacgagatgatc

SEQ ID NO: 169 - RNA, 20 nt, synthetic construct
  source 1..20: mol_type = other RNA
accttgaggatatcaacgag

SEQ ID NO: 170 - RNA, 20 nt, synthetic construct
  source 1..20: mol_type = other RNA
atatcctcaaggtcacccac

SEQ ID NO: 171 - RNA, 20 nt, synthetic construct
  source 1..20: mol_type = other RNA
ggtgaccttgaggatatcaa

SEQ ID NO: 172 - RNA, 20 nt, synthetic construct
  source 1..20: mol_type = other RNA
tcctcaaggtcacccaccat

SEQ ID NO: 173 - RNA, 20 nt, synthetic construct
  source 1..20: mol_type = other RNA
gtgggtgaccttgaggatat

SEQ ID NO: 174 - RNA, 20 nt, synthetic construct
  source 1..20: mol_type = other RNA
atggtgggtgaccttgagga

SEQ ID NO: 175 - RNA, 20 nt, synthetic construct
  source 1..20: mol_type = other RNA
aaatcacagagggtgatggt

SEQ ID NO: 176 - RNA, 20 nt, synthetic construct
  source 1..20: mol_type = other RNA
taaaatcacagagggtgatg

SEQ ID NO: 177 - RNA, 20 nt, synthetic construct
  source 1..20: mol_type = other RNA
atcaagttataaaatcacag

SEQ ID NO: 178 - RNA, 20 nt, synthetic construct
  source 1..20: mol_type = other RNA
attttataacttgatcaagc

SEQ ID NO: 179 - RNA, 20 nt, synthetic construct
  source 1..20: mol_type = other RNA
cttgatcaagttataaaatc

SEQ ID NO: 180 - RNA, 20 nt, synthetic construct
  source 1..20: mol_type = other RNA
taacttgatcaagcagagaa

SEQ ID NO: 181 - RNA, 20 nt, synthetic construct
  source 1..20: mol_type = other RNA
acttgatcaagcagagaaag

SEQ ID NO: 182 - RNA, 20 nt, synthetic construct
  source 1..20: mol_type = other RNA
atcaagcagagaaagccagt

SEQ ID NO: 183 - RNA, 20 nt, synthetic construct
  source 1..20: mol_type = other RNA
gctttctctgcttgatcaag

SEQ ID NO: 184 - RNA, 20 nt, synthetic construct
  source 1..20: mol_type = other RNA
ccgactggctttctctgctt

SEQ ID NO: 185 - RNA, 20 nt, synthetic construct
  source 1..20: mol_type = other RNA
gacagaacttaccgactggc

SEQ ID NO: 186 - RNA, 20 nt, synthetic construct
  source 1..20: mol_type = other RNA
agttctgtccaagcccggtt

SEQ ID NO: 187 - RNA, 20 nt, synthetic construct
  source 1..20: mol_type = other RNA
tccaagcccggttgaaatct

SEQ ID NO: 188 - RNA, 20 nt, synthetic construct
  source 1..20: mol_type = other RNA
gcagatttcaaccgggcttg

SEQ ID NO: 189 - RNA, 20 nt, synthetic construct
  source 1..20: mol_type = other RNA
ctctggcagatttcaaccgg

SEQ ID NO: 190 - RNA, 20 nt, synthetic construct
  source 1..20: mol_type = other RNA
aaatctgccagagcaggtac

SEQ ID NO: 191 - RNA, 20 nt, synthetic construct
  source 1..20: mol_type = other RNA
cctgctctggcagatttcaa

SEQ ID NO: 192 - RNA, 20 nt, synthetic construct
  source 1..20: mol_type = other RNA
ccagagcaggtacctccaac

SEQ ID NO: 193 - RNA, 20 nt, synthetic construct
  source 1..20: mol_type = other RNA
gaggtacctgctctggcaga

SEQ ID NO: 194 - RNA, 20 nt, synthetic construct
  source 1..20: mol_type = other RNA
ttggaggtacctgctctggc

SEQ ID NO: 195 - RNA, 20 nt, synthetic construct
  source 1..20: mol_type = other RNA
atgttggaggtacctgctct

SEQ ID NO: 196 - RNA, 20 nt, synthetic construct
  source 1..20: mol_type = other RNA
cctccaacatcaaggaagat

SEQ ID NO: 197 - RNA, 20 nt, synthetic construct
  source 1..20: mol_type = other RNA
ccatcttccttgatgttgga

SEQ ID NO: 198 - RNA, 20 nt, synthetic construct
  source 1..20: mol_type = other RNA
gaaatgccatcttccttgat

SEQ ID NO: 199 - RNA, 20 nt, synthetic construct
  source 1..20: mol_type = other RNA
gcatttctagtttggagatg

SEQ ID NO: 200 - RNA, 20 nt, synthetic construct
  source 1..20: mol_type = other RNA
ccatctccaaactagaaatg

SEQ ID NO: 201 - RNA, 20 nt, synthetic construct
  source 1..20: mol_type = other RNA
gtttggagatggcagtttcc

SEQ ID NO: 202 - RNA, 20 nt, synthetic construct
  source 1..20: mol_type = other RNA
gagatggcagtttccttagt

SEQ ID NO: 203 - RNA, 20 nt, synthetic construct
  source 1..20: mol_type = other RNA
aggaaactgccatctccaaa

SEQ ID NO: 204 - RNA, 20 nt, synthetic construct
  source 1..20: mol_type = other RNA
ctaaggaaactgccatctcc

SEQ ID NO: 205 - RNA, 20 nt, synthetic construct
  source 1..20: mol_type = other RNA
gcagtttccttagtaaccac

SEQ ID NO: 206 - RNA, 20 nt, synthetic construct
  source 1..20: mol_type = other RNA
gttactaaggaaactgccat

SEQ ID NO: 207 - RNA, 20 nt, synthetic construct
  source 1..20: mol_type = other RNA
tggttactaaggaaactgcc

SEQ ID NO: 208 - RNA, 20 nt, synthetic construct
  source 1..20: mol_type = other RNA
gtaaccacaggttgtgtcac

SEQ ID NO: 209 - RNA, 20 nt, synthetic construct
  source 1..20: mol_type = other RNA
acacaacctgtggttactaa

SEQ ID NO: 210 - RNA, 20 nt, synthetic construct
  source 1..20: mol_type = other RNA
accacaggttgtgtcaccag

SEQ ID NO: 211 - RNA, 20 nt, synthetic construct
  source 1..20: mol_type = other RNA
gtgacacaacctgtggttac

SEQ ID NO: 212 - RNA, 20 nt, synthetic construct
  source 1..20: mol_type = other RNA
ctctggtgacacaacctgtg

SEQ ID NO: 213 - RNA, 20 nt, synthetic construct
  source 1..20: mol_type = other RNA
ttactctggtgacacaacct

SEQ ID NO: 214 - RNA, 20 nt, synthetic construct
  source 1..20: mol_type = other RNA
tgtcaccagagtaacagtct

SEQ ID NO: 215 - RNA, 20 nt, synthetic construct
  source 1..20: mol_type = other RNA
tcaccagagtaacagtctga

SEQ ID NO: 216 - RNA, 20 nt, synthetic construct
  source 1..20: mol_type = other RNA
ctcagactgttactctggtg

SEQ ID NO: 217 - RNA, 20 nt, synthetic construct
  source 1..20: mol_type = other RNA
caaacagctgtcagacagaa

SEQ ID NO: 218 - RNA, 20 nt, synthetic construct
  source 1..20: mol_type = other RNA
tctgacagctgtttgcagac

SEQ ID NO: 219 - RNA, 20 nt, synthetic construct
  source 1..20: mol_type = other RNA
acagctgtttgcagacctcc

SEQ ID NO: 220 - RNA, 20 nt, synthetic construct
  source 1..20: mol_type = other RNA
tttgcagacctcctgccacc

SEQ ID NO: 221 - RNA, 20 nt, synthetic construct
  source 1..20: mol_type = other RNA
gcaggaggtctgcaaacagc

SEQ ID NO: 222 - RNA, 20 nt, synthetic construct
  source 1..20: mol_type = other RNA
cagacctcctgccaccgcag

SEQ ID NO: 223 - RNA, 20 nt, synthetic construct
  source 1..20: mol_type = other RNA
cggtggcaggaggtctgcaa

SEQ ID NO: 224 - RNA, 20 nt, synthetic construct
  source 1..20: mol_type = other RNA
aatctgcggtggcaggaggt

SEQ ID NO: 225 - RNA, 20 nt, synthetic construct
  source 1..20: mol_type = other RNA
ccaccgcagattcaggcttc

SEQ ID NO: 226 - RNA, 20 nt, synthetic construct
  source 1..20: mol_type = other RNA
ggaagcctgaatctgcggtg

SEQ ID NO: 227 - RNA, 20 nt, synthetic construct
  source 1..20: mol_type = other RNA
caggaaaaattgggaagcct

SEQ ID NO: 228 - RNA, 20 nt, synthetic construct
  source 1..20: mol_type = other RNA
ttctacaggaaaaattggga

SEQ ID NO: 229 - RNA, 20 nt, synthetic construct
  source 1..20: mol_type = other RNA
ccagtattctacaggaaaaa

SEQ ID NO: 230 - RNA, 20 nt, synthetic construct
  source 1..20: mol_type = other RNA
tagaatactggcatctgttt

SEQ ID NO: 231 - RNA, 20 nt, synthetic construct
  source 1..20: mol_type = other RNA
gaatactggcatctgttttt

SEQ ID NO: 232 - RNA, 20 nt, synthetic construct
  source 1..20: mol_type = other RNA
ctggcatctgtttttgagga

SEQ ID NO: 233 - RNA, 20 nt, synthetic construct
  source 1..20: mol_type = other RNA
gcatctgtttttgaggattg

SEQ ID NO: 234 - RNA, 20 nt, synthetic construct
  source 1..20: mol_type = other RNA
tttttgaggattgctgaatt

SEQ ID NO: 235 - RNA, 20 nt, synthetic construct
  source 1..20: mol_type = other RNA
attcagcaatcctcaaaaac

SEQ ID NO: 236 - RNA, 20 nt, synthetic construct
  source 1..20: mol_type = other RNA
aggattgctgaattatttct

SEQ ID NO: 237 - RNA, 20 nt, synthetic construct
  source 1..20: mol_type = other RNA
ctgaattatttcttccccag

SEQ ID NO: 238 - RNA, 20 nt, synthetic construct
  source 1..20: mol_type = other RNA
gggaagaaataattcagcaa

SEQ ID NO: 239 - RNA, 20 nt, synthetic construct
  source 1..20: mol_type = other RNA
aattatttcttccccagttg

SEQ ID NO: 240 - RNA, 20 nt, synthetic construct
  source 1..20: mol_type = other RNA
caactggggaagaaataatt

SEQ ID NO: 241 - RNA, 20 nt, synthetic construct
  source 1..20: mol_type = other RNA
tttcttccccagttgcattc

SEQ ID NO: 242 - RNA, 20 nt, synthetic construct
  source 1..20: mol_type = other RNA
aatgcaactggggaagaaat

SEQ ID NO: 243 - RNA, 20 nt, synthetic construct
  source 1..20: mol_type = other RNA
tcagaacattgaatgcaact

SEQ ID NO: 244 - RNA, 20 nt, synthetic construct
  source 1..20: mol_type = other RNA
cattcaatgttctgacaaca

SEQ ID NO: 245 - RNA, 20 nt, synthetic construct
  source 1..20: mol_type = other RNA
ttgtcagaacattgaatgca

SEQ ID NO: 246 - RNA, 20 nt, synthetic construct
  source 1..20: mol_type = other RNA
ttctgacaacagtttgccgc

SEQ ID NO: 247 - RNA, 20 nt, synthetic construct
  source 1..20: mol_type = other RNA
acaacagtttgccgctgccc

SEQ ID NO: 248 - RNA, 20 nt, synthetic construct
  source 1..20: mol_type = other RNA
ggcagcggcaaactgttgtc

SEQ ID NO: 249 - RNA, 20 nt, synthetic construct
  source 1..20: mol_type = other RNA
gcattgggcagcggcaaact

SEQ ID NO: 250 - RNA, 20 nt, synthetic construct
  source 1..20: mol_type = other RNA
ccgctgcccaatgccatcct

SEQ ID NO: 251 - RNA, 20 nt, synthetic construct
  source 1..20: mol_type = other RNA
cccaatgccatcctggagtt

SEQ ID NO: 252 - RNA, 20 nt, synthetic construct
  source 1..20: mol_type = other RNA
agataccatttgtatttagc

SEQ ID NO: 253 - RNA, 20 nt, synthetic construct
  source 1..20: mol_type = other RNA
aatacaaatggtatcttaag

SEQ ID NO: 254 - RNA, 20 nt, synthetic construct
  source 1..20: mol_type = other RNA
ctaaatacaaatggtatctt

SEQ ID NO: 255 - RNA, 20 nt, synthetic construct
  source 1..20: mol_type = other RNA
ccatttgtatttagcatgtt

SEQ ID NO: 256 - RNA, 20 nt, synthetic construct
  source 1..20: mol_type = other RNA
ggaacatgctaaatacaaat

SEQ ID NO: 257 - RNA, 20 nt, synthetic construct
  source 1..20: mol_type = other RNA
tatttagcatgttcccaatt

SEQ ID NO: 258 - RNA, 20 nt, synthetic construct
  source 1..20: mol_type = other RNA
tttagcatgttcccaattct

SEQ ID NO: 259 - RNA, 20 nt, synthetic construct
  source 1..20: mol_type = other RNA
gcatgttcccaattctcagg

SEQ ID NO: 260 - RNA, 20 nt, synthetic construct
  source 1..20: mol_type = other RNA
agaattgggaacatgctaaa

SEQ ID NO: 261 - RNA, 20 nt, synthetic construct
  source 1..20: mol_type = other RNA
ttcccaattctcaggaattt

SEQ ID NO: 262 - RNA, 20 nt, synthetic construct
  source 1..20: mol_type = other RNA
tgtctttctgagaaactgtt

SEQ ID NO: 263 - RNA, 20 nt, synthetic construct
  source 1..20: mol_type = other RNA
tctttctgagaaactgttca

SEQ ID NO: 264 - RNA, 20 nt, synthetic construct
  source 1..20: mol_type = other RNA
aacagtttctcagaaagaca

SEQ ID NO: 265 - RNA, 20 nt, synthetic construct
  source 1..20: mol_type = other RNA
agaaactgttcagcttctgt

SEQ ID NO: 266 - RNA, 20 nt, synthetic construct
  source 1..20: mol_type = other RNA
acagaagctgaacagtttct

SEQ ID NO: 267 - RNA, 20 nt, synthetic construct
  source 1..20: mol_type = other RNA
ttcagcttctgttagccact

SEQ ID NO: 268 - RNA, 20 nt, synthetic construct
  source 1..20: mol_type = other RNA
gctaacagaagctgaacagt

SEQ ID NO: 269 - RNA, 20 nt, synthetic construct
  source 1..20: mol_type = other RNA
atcagtggctaacagaagct

SEQ ID NO: 270 - RNA, 20 nt, synthetic construct
  source 1..20: mol_type = other RNA
ttagccactgattaaatatc

SEQ ID NO: 271 - RNA, 20 nt, synthetic construct
  source 1..20: mol_type = other RNA
tttaatcagtggctaacaga

SEQ ID NO: 272 - RNA, 20 nt, synthetic construct
  source 1..20: mol_type = other RNA
gccactgattaaatatcttt

SEQ ID NO: 273 - RNA, 20 nt, synthetic construct
  source 1..20: mol_type = other RNA
aagatatttaatcagtggct

SEQ ID NO: 274 - RNA, 20 nt, synthetic construct
  source 1..20: mol_type = other RNA
taaagatatttaatcagtgg

SEQ ID NO: 275 - RNA, 20 nt, synthetic construct
  source 1..20: mol_type = other RNA
tatcataatgaaaacgccgc

SEQ ID NO: 276 - RNA, 20 nt, synthetic construct
  source 1..20: mol_type = other RNA
tcataatgaaaacgccgcca

SEQ ID NO: 277 - RNA, 20 nt, synthetic construct
  source 1..20: mol_type = other RNA
gcggcgttttcattatgata

SEQ ID NO: 278 - RNA, 20 nt, synthetic construct
  source 1..20: mol_type = other RNA
atgaaaacgccgccatttct

SEQ ID NO: 279 - RNA, 20 nt, synthetic construct
  source 1..20: mol_type = other RNA
aaaacgccgccatttctcaa

SEQ ID NO: 280 - RNA, 20 nt, synthetic construct
  source 1..20: mol_type = other RNA
agaaatggcggcgttttcat

SEQ ID NO: 281 - RNA, 20 nt, synthetic construct
  source 1..20: mol_type = other RNA
ttgagaaatggcggcgtttt

SEQ ID NO: 282 - RNA, 20 nt, synthetic construct
  source 1..20: mol_type = other RNA
acagatctgttgagaaatgg

SEQ ID NO: 283 - RNA, 68 nt, synthetic construct
  source 1..68: mol_type = other RNA
acagcttatttggaagctgaaatgtgaggtttataacactcacaagaatcctgaaaaggatgccaaac

SEQ ID NO: 284-465 - skipped (182 entries)

SEQ ID NO: 466 - RNA, 88 nt, synthetic construct
  source 1..88: mol_type = other RNA
acagcttatttggaagctgaaatgtgaggtttataacactcacaagaatcctgaaaaggatgccaaacgtttctgtgattttcttttg

SEQ ID NO: 467 - RNA, 88 nt, synthetic construct
  source 1..88: mol_type = other RNA
acagcttatttggaagctgaaatgtgaggtttataacactcacaagaatcctgaaaaggatgccaaactgattttcttttggattgca

SEQ ID NO: 468 - RNA, 88 nt, synthetic construct
  source 1..88: mol_type = other RNA
acagcttatttggaagctgaaatgtgaggtttataacactcacaagaatcctgaaaaggatgccaaacattttcttttggattgcatc

SEQ ID NO: 469 - RNA, 88 nt, synthetic construct
  source 1..88: mol_type = other RNA
acagcttatttggaagctgaaatgtgaggtttataacactcacaagaatcctgaaaaggatgccaaaccaatccaaaagaaaatcaca

SEQ ID NO: 470 - RNA, 88 nt, synthetic construct
  source 1..88: mol_type = other RNA
acagcttatttggaagctgaaatgtgaggtttataacactcacaagaatcctgaaaaggatgccaaacgatgcaatccaaaagaaaat

SEQ ID NO: 471 - RNA, 88 nt, synthetic construct
  source 1..88: mol_type = other RNA
acagcttatttggaagctgaaatgtgaggtttataacactcacaagaatcctgaaaaggatgccaaaccagtagatgcaatccaaaag

SEQ ID NO: 472 - RNA, 88 nt, synthetic construct
  source 1..88: mol_type = other RNA
acagcttatttggaagctgaaatgtgaggtttataacactcacaagaatcctgaaaaggatgccaaacgattgcatctactgtatagg

SEQ ID NO: 473 - RNA, 88 nt, synthetic construct
  source 1..88: mol_type = other RNA
acagcttatttggaagctgaaatgtgaggtttataacactcacaagaatcctgaaaaggatgccaaactacagtagatgcaatccaaa

SEQ ID NO: 474 - RNA, 88 nt, synthetic construct
  source 1..88: mol_type = other RNA
acagcttatttggaagctgaaatgtgaggtttataacactcacaagaatcctgaaaaggatgccaaaccatctactgtatagggaccc

SEQ ID NO: 475 - RNA, 88 nt, synthetic construct
  source 1..88: mol_type = other RNA
acagcttatttggaagctgaaatgtgaggtttataacactcacaagaatcctgaaaaggatgccaaacctgtatagggaccctccttc

SEQ ID NO: 476 - RNA, 88 nt, synthetic construct
  source 1..88: mol_type = other RNA
acagcttatttggaagctgaaatgtgaggtttataacactcacaagaatcctgaaaaggatgccaaactatagggaccctccttccat

SEQ ID NO: 477 - RNA, 88 nt, synthetic construct
  source 1..88: mol_type = other RNA
acagcttatttggaagctgaaatgtgaggtttataacactcacaagaatcctgaaaaggatgccaaacgaaggagggtccctatacag

SEQ ID NO: 478 - RNA, 88 nt, synthetic construct
  source 1..88: mol_type = other RNA
acagcttatttggaagctgaaatgtgaggtttataacactcacaagaatcctgaaaaggatgccaaactagggaccctccttccatga

SEQ ID NO: 479 - RNA, 88 nt, synthetic construct
  source 1..88: mol_type = other RNA
acagcttatttggaagctgaaatgtgaggtttataacactcacaagaatcctgaaaaggatgccaaacgggaccctccttccatgact

SEQ ID NO: 480 - RNA, 88 nt, synthetic construct
  source 1..88: mol_type = other RNA
acagcttatttggaagctgaaatgtgaggtttataacactcacaagaatcctgaaaaggatgccaaacagtcatggaaggagggtccc

SEQ ID NO: 481 - RNA, 88 nt, synthetic construct
  source 1..88: mol_type = other RNA
acagcttatttggaagctgaaatgtgaggtttataacactcacaagaatcctgaaaaggatgccaaacactcaagcttggctctggcc

SEQ ID NO: 482 - RNA, 88 nt, synthetic construct
  source 1..88: mol_type = other RNA
acagcttatttggaagctgaaatgtgaggtttataacactcacaagaatcctgaaaaggatgccaaacggacaggccagagccaagct

SEQ ID NO: 483 - RNA, 88 nt, synthetic construct
  source 1..88: mol_type = other RNA
acagcttatttggaagctgaaatgtgaggtttataacactcacaagaatcctgaaaaggatgccaaacgctctggcctgtcctaagac

SEQ ID NO: 484 - RNA, 88 nt, synthetic construct
  source 1..88: mol_type = other RNA
acagcttatttggaagctgaaatgtgaggtttataacactcacaagaatcctgaaaaggatgccaaacgcctgtcctaagacctgctc

SEQ ID NO: 485 - RNA, 88 nt, synthetic construct
  source 1..88: mol_type = other RNA
acagcttatttggaagctgaaatgtgaggtttataacactcacaagaatcctgaaaaggatgccaaacagcaggtcttaggacaggcc

SEQ ID NO: 486 - RNA, 88 nt, synthetic construct
  source 1..88: mol_type = other RNA
acagcttatttggaagctgaaatgtgaggtttataacactcacaagaatcctgaaaaggatgccaaactcctaagacctgctcagctt

SEQ ID NO: 487 - RNA, 88 nt, synthetic construct
  source 1..88: mol_type = other RNA
acagcttatttggaagctgaaatgtgaggtttataacactcacaagaatcctgaaaaggatgccaaacagacctgctcagcttcttcc

SEQ ID NO: 488 - RNA, 88 nt, synthetic construct
  source 1..88: mol_type = other RNA
acagcttatttggaagctgaaatgtgaggtttataacactcacaagaatcctgaaaaggatgccaaacaggaagaagctgagcaggtc

SEQ ID NO: 489 - RNA, 88 nt, synthetic construct
  source 1..88: mol_type = other RNA
acagcttatttggaagctgaaatgtgaggtttataacactcacaagaatcctgaaaaggatgccaaacctcagcttcttccttagctt

SEQ ID NO: 490 - RNA, 88 nt, synthetic construct
  source 1..88: mol_type = other RNA
acagcttatttggaagctgaaatgtgaggtttataacactcacaagaatcctgaaaaggatgccaaacgaagctaaggaagaagctga

SEQ ID NO: 491 - RNA, 88 nt, synthetic construct
  source 1..88: mol_type = other RNA
acagcttatttggaagctgaaatgtgaggtttataacactcacaagaatcctgaaaaggatgccaaacgctggaagctaaggaagaag

SEQ ID NO: 492 - RNA, 88 nt, synthetic construct
  source 1..88: mol_type = other RNA
acagcttatttggaagctgaaatgtgaggtttataacactcacaagaatcctgaaaaggatgccaaacgcttccagccattgtgttga

SEQ ID NO: 493 - RNA, 88 nt, synthetic construct
  source 1..88: mol_type = other RNA
acagcttatttggaagctgaaatgtgaggtttataacactcacaagaatcctgaaaaggatgccaaacaaggattcaacacaatggct

SEQ ID NO: 494 - RNA, 88 nt, synthetic construct
  source 1..88: mol_type = other RNA
acagcttatttggaagctgaaatgtgaggtttataacactcacaagaatcctgaaaaggatgccaaacttaaaggattcaacacaatg

SEQ ID NO: 495 - RNA, 88 nt, synthetic construct
  source 1..88: mol_type = other RNA
acagcttatttggaagctgaaatgtgaggtttataacactcacaagaatcctgaaaaggatgccaaactgttgaatcctttaacattt

SEQ ID NO: 496 - RNA, 88 nt, synthetic construct
  source 1..88: mol_type = other RNA
acagcttatttggaagctgaaatgtgaggtttataacactcacaagaatcctgaaaaggatgccaaacttgaatcctttaacatttca

SEQ ID NO: 497 - RNA, 88 nt, synthetic construct
  source 1..88: mol_type = other RNA
acagcttatttggaagctgaaatgtgaggtttataacactcacaagaatcctgaaaaggatgccaaacaaatgttaaaggattcaaca

SEQ ID NO: 498 - RNA, 88 nt, synthetic construct
  source 1..88: mol_type = other RNA
acagcttatttggaagctgaaatgtgaggtttataacactcacaagaatcctgaaaaggatgccaaacaatcctttaacatttcattc

SEQ ID NO: 499 - RNA, 88 nt, synthetic construct
  source 1..88: mol_type = other RNA
acagcttatttggaagctgaaatgtgaggtttataacactcacaagaatcctgaaaaggatgccaaacaatgaaatgttaaaggattc

SEQ ID NO: 500 - RNA, 88 nt, synthetic construct
  source 1..88: mol_type = other RNA
acagcttatttggaagctgaaatgtgaggtttataacactcacaagaatcctgaaaaggatgccaaacacatttcattcaactgttgc

SEQ ID NO: 501 - RNA, 88 nt, synthetic construct
  source 1..88: mol_type = other RNA
acagcttatttggaagctgaaatgtgaggtttataacactcacaagaatcctgaaaaggatgccaaacttgcctccggttctgaaggt

SEQ ID NO: 502 - RNA, 88 nt, synthetic construct
  source 1..88: mol_type = other RNA
acagcttatttggaagctgaaatgtgaggtttataacactcacaagaatcctgaaaaggatgccaaaccctccggttctgaaggtgtt

SEQ ID NO: 503 - RNA, 88 nt, synthetic construct
  source 1..88: mol_type = other RNA
acagcttatttggaagctgaaatgtgaggtttataacactcacaagaatcctgaaaaggatgccaaaccaagaacaccttcagaaccg

SEQ ID NO: 504 - RNA, 88 nt, synthetic construct
  source 1..88: mol_type = other RNA
acagcttatttggaagctgaaatgtgaggtttataacactcacaagaatcctgaaaaggatgccaaacaaggtgttcttgtacttcat

SEQ ID NO: 505 - RNA, 88 nt, synthetic construct
  source 1..88: mol_type = other RNA
acagcttatttggaagctgaaatgtgaggtttataacactcacaagaatcctgaaaaggatgccaaacaagtacaagaacaccttcag

SEQ ID NO: 506 - RNA, 88 nt, synthetic construct
  source 1..88: mol_type = other RNA
acagcttatttggaagctgaaatgtgaggtttataacactcacaagaatcctgaaaaggatgccaaacttcttgtacttcatcccact

SEQ ID NO: 507 - RNA, 88 nt, synthetic construct
  source 1..88: mol_type = other RNA
acagcttatttggaagctgaaatgtgaggtttataacactcacaagaatcctgaaaaggatgccaaacggatgaagtacaagaacacc

SEQ ID NO: 508 - RNA, 88 nt, synthetic construct
  source 1..88: mol_type = other RNA
acagcttatttggaagctgaaatgtgaggtttataacactcacaagaatcctgaaaaggatgccaaactacttcatcccactgattct

SEQ ID NO: 509 - RNA, 88 nt, synthetic construct
  source 1..88: mol_type = other RNA
acagcttatttggaagctgaaatgtgaggtttataacactcacaagaatcctgaaaaggatgccaaaccttcatcccactgattctga

SEQ ID NO: 510 - RNA, 88 nt, synthetic construct
  source 1..88: mol_type = other RNA
acagcttatttggaagctgaaatgtgaggtttataacactcacaagaatcctgaaaaggatgccaaacgaacaatcattacggatcga

SEQ ID NO: 511 - RNA, 88 nt, synthetic construct
  source 1..88: mol_type = other RNA
acagcttatttggaagctgaaatgtgaggtttataacactcacaagaatcctgaaaaggatgccaaacatgattgttctagcctcttg

SEQ ID NO: 512 - RNA, 88 nt, synthetic construct
  source 1..88: mol_type = other RNA
acagcttatttggaagctgaaatgtgaggtttataacactcacaagaatcctgaaaaggatgccaaacattgttctagcctcttgatt

SEQ ID NO: 513 - RNA, 88 nt, synthetic construct
  source 1..88: mol_type = other RNA
acagcttatttggaagctgaaatgtgaggtttataacactcacaagaatcctgaaaaggatgccaaacttctagcctcttgattgctg

SEQ ID NO: 514 - RNA, 88 nt, synthetic construct
  source 1..88: mol_type = other RNA
acagcttatttggaagctgaaatgtgaggtttataacactcacaagaatcctgaaaaggatgccaaacgcctcttgattgctggtctt

SEQ ID NO: 515 - RNA, 88 nt, synthetic construct
  source 1..88: mol_type = other RNA
acagcttatttggaagctgaaatgtgaggtttataacactcacaagaatcctgaaaaggatgccaaacattgctggtcttgtttttca

SEQ ID NO: 516 - RNA, 88 nt, synthetic construct
  source 1..88: mol_type = other RNA
acagcttatttggaagctgaaatgtgaggtttataacactcacaagaatcctgaaaaggatgccaaacaaaaacaagaccagcaatca

SEQ ID NO: 517 - RNA, 88 nt, synthetic construct
  source 1..88: mol_type = other RNA
acagcttatttggaagctgaaatgtgaggtttataacactcacaagaatcctgaaaaggatgccaaacctggtcttgtttttcaaatt

SEQ ID NO: 518 - RNA, 88 nt, synthetic construct
  source 1..88: mol_type = other RNA
acagcttatttggaagctgaaatgtgaggtttataacactcacaagaatcctgaaaaggatgccaaacgtcttgtttttcaaattttg

SEQ ID NO: 519 - RNA, 88 nt, synthetic construct
  source 1..88: mol_type = other RNA
acagcttatttggaagctgaaatgtgaggtttataacactcacaagaatcctgaaaaggatgccaaactttttcaaattttgggcagc

SEQ ID NO: 520 - RNA, 88 nt, synthetic construct
  source 1..88: mol_type = other RNA
acagcttatttggaagctgaaatgtgaggtttataacactcacaagaatcctgaaaaggatgccaaaccccaaaatttgaaaaacaag

SEQ ID NO: 521 - RNA, 88 nt, synthetic construct
  source 1..88: mol_type = other RNA
acagcttatttggaagctgaaatgtgaggtttataacactcacaagaatcctgaaaaggatgccaaacccgctgcccaaaatttgaaa

SEQ ID NO: 522 - RNA, 88 nt, synthetic construct
  source 1..88: mol_type = other RNA
acagcttatttggaagctgaaatgtgaggtttataacactcacaagaatcctgaaaaggatgccaaacggcagcggtaatgagttctt

SEQ ID NO: 523 - RNA, 88 nt, synthetic construct
  source 1..88: mol_type = other RNA
acagcttatttggaagctgaaatgtgaggtttataacactcacaagaatcctgaaaaggatgccaaacgaagaactcattaccgctgc

SEQ ID NO: 524 - RNA, 88 nt, synthetic construct
  source 1..88: mol_type = other RNA
acagcttatttggaagctgaaatgtgaggtttataacactcacaagaatcctgaaaaggatgccaaacatgagttcttccaactgggg

SEQ ID NO: 525 - RNA, 88 nt, synthetic construct
  source 1..88: mol_type = other RNA
acagcttatttggaagctgaaatgtgaggtttataacactcacaagaatcctgaaaaggatgccaaacagttcttccaactggggacg

SEQ ID NO: 526 - RNA, 88 nt, synthetic construct
  source 1..88: mol_type = other RNA
acagcttatttggaagctgaaatgtgaggtttataacactcacaagaatcctgaaaaggatgccaaacgaacagaggcgtccccagtt

SEQ ID NO: 527 - RNA, 88 nt, synthetic construct
  source 1..88: mol_type = other RNA
acagcttatttggaagctgaaatgtgaggtttataacactcacaagaatcctgaaaaggatgccaaacgggacgcctctgttccaaat

SEQ ID NO: 528 - RNA, 88 nt, synthetic construct
  source 1..88: mol_type = other RNA
acagcttatttggaagctgaaatgtgaggtttataacactcacaagaatcctgaaaaggatgccaaaccaggatttggaacagaggcg

SEQ ID NO: 529 - RNA, 88 nt, synthetic construct
  source 1..88: mol_type = other RNA
acagcttatttggaagctgaaatgtgaggtttataacactcacaagaatcctgaaaaggatgccaaacttccaaatcctgcattgttg

SEQ ID NO: 530 - RNA, 88 nt, synthetic construct
  source 1..88: mol_type = other RNA
acagcttatttggaagctgaaatgtgaggtttataacactcacaagaatcctgaaaaggatgccaaaccttgatgatcatctcgttga

SEQ ID NO: 531 - RNA, 88 nt, synthetic construct
  source 1..88: mol_type = other RNA
acagcttatttggaagctgaaatgtgaggtttataacactcacaagaatcctgaaaaggatgccaaacatgatcatctcgttgatatc

SEQ ID NO: 532 - RNA, 88 nt, synthetic construct
  source 1..88: mol_type = other RNA
acagcttatttggaagctgaaatgtgaggtttataacactcacaagaatcctgaaaaggatgccaaactcaacgagatgatcatcaag

SEQ ID NO: 533 - RNA, 88 nt, synthetic construct
  source 1..88: mol_type = other RNA
acagcttatttggaagctgaaatgtgaggtttataacactcacaagaatcctgaaaaggatgccaaacatcatctcgttgatatcctc

SEQ ID NO: 534 - RNA, 88 nt, synthetic construct
  source 1..88: mol_type = other RNA
acagcttatttggaagctgaaatgtgaggtttataacactcacaagaatcctgaaaaggatgccaaacaggatatcaacgagatgatc

SEQ ID NO: 535 - RNA, 88 nt, synthetic construct
  source 1..88: mol_type = other RNA
acagcttatttggaagctgaaatgtgaggtttataacactcacaagaatcctgaaaaggatgccaaacaccttgaggatatcaacgag

SEQ ID NO: 536 - RNA, 88 nt, synthetic construct
  source 1..88: mol_type = other RNA
acagcttatttggaagctgaaatgtgaggtttataacactcacaagaatcctgaaaaggatgccaaacatatcctcaaggtcacccac

SEQ ID NO: 537 - RNA, 88 nt, synthetic construct
  source 1..88: mol_type = other RNA
acagcttatttggaagctgaaatgtgaggtttataacactcacaagaatcctgaaaaggatgccaaacggtgaccttgaggatatcaa

SEQ ID NO: 538 - RNA, 88 nt, synthetic construct
  source 1..88: mol_type = other RNA
acagcttatttggaagctgaaatgtgaggtttataacactcacaagaatcctgaaaaggatgccaaactcctcaaggtcacccaccat

SEQ ID NO: 539 - RNA, 88 nt, synthetic construct
  source 1..88: mol_type = other RNA
acagcttatttggaagctgaaatgtgaggtttataacactcacaagaatcctgaaaaggatgccaaacgtgggtgaccttgaggatat

SEQ ID NO: 540 - RNA, 88 nt, synthetic construct
  source 1..88: mol_type = other RNA
acagcttatttggaagctgaaatgtgaggtttataacactcacaagaatcctgaaaaggatgccaaacatggtgggtgaccttgagga

SEQ ID NO: 541 - RNA, 88 nt, synthetic construct
  source 1..88: mol_type = other RNA
acagcttatttggaagctgaaatgtgaggtttataacactcacaagaatcctgaaaaggatgccaaacaaatcacagagggtgatggt

SEQ ID NO: 542 - RNA, 88 nt, synthetic construct
  source 1..88: mol_type = other RNA
acagcttatttggaagctgaaatgtgaggtttataacactcacaagaatcctgaaaaggatgccaaactaaaatcacagagggtgatg

SEQ ID NO: 543 - RNA, 88 nt, synthetic construct
  source 1..88: mol_type = other RNA
acagcttatttggaagctgaaatgtgaggtttataacactcacaagaatcctgaaaaggatgccaaacatcaagttataaaatcacag

SEQ ID NO: 544 - RNA, 88 nt, synthetic construct
  source 1..88: mol_type = other RNA
acagcttatttggaagctgaaatgtgaggtttataacactcacaagaatcctgaaaaggatgccaaacattttataacttgatcaagc

SEQ ID NO: 545 - RNA, 88 nt, synthetic construct
  source 1..88: mol_type = other RNA
acagcttatttggaagctgaaatgtgaggtttataacactcacaagaatcctgaaaaggatgccaaaccttgatcaagttataaaatc

SEQ ID NO: 546 - RNA, 88 nt, synthetic construct
  source 1..88: mol_type = other RNA
acagcttatttggaagctgaaatgtgaggtttataacactcacaagaatcctgaaaaggatgccaaactaacttgatcaagcagagaa

SEQ ID NO: 547 - RNA, 88 nt, synthetic construct
  source 1..88: mol_type = other RNA
acagcttatttggaagctgaaatgtgaggtttataacactcacaagaatcctgaaaaggatgccaaacacttgatcaagcagagaaag

SEQ ID NO: 548 - RNA, 88 nt, synthetic construct
  source 1..88: mol_type = other RNA
acagcttatttggaagctgaaatgtgaggtttataacactcacaagaatcctgaaaaggatgccaaacatcaagcagagaaagccagt

SEQ ID NO: 549 - RNA, 88 nt, synthetic construct
  source 1..88: mol_type = other RNA
acagcttatttggaagctgaaatgtgaggtttataacactcacaagaatcctgaaaaggatgccaaacgctttctctgcttgatcaag

SEQ ID NO: 550 - RNA, 88 nt, synthetic construct
  source 1..88: mol_type = other RNA
acagcttatttggaagctgaaatgtgaggtttataacactcacaagaatcctgaaaaggatgccaaacccgactggctttctctgctt

SEQ ID NO: 551 - RNA, 88 nt, synthetic construct
  source 1..88: mol_type = other RNA
acagcttatttggaagctgaaatgtgaggtttataacactcacaagaatcctgaaaaggatgccaaacgacagaacttaccgactggc

SEQ ID NO: 552 - RNA, 88 nt, synthetic construct
  source 1..88: mol_type = other RNA
acagcttatttggaagctgaaatgtgaggtttataacactcacaagaatcctgaaaaggatgccaaacagttctgtccaagcccggtt

SEQ ID NO: 553 - RNA, 88 nt, synthetic construct
  source 1..88: mol_type = other RNA
acagcttatttggaagctgaaatgtgaggtttataacactcacaagaatcctgaaaaggatgccaaactccaagcccggttgaaatct

SEQ ID NO: 554 - RNA, 88 nt, synthetic construct
  source 1..88: mol_type = other RNA
acagcttatttggaagctgaaatgtgaggtttataacactcacaagaatcctgaaaaggatgccaaacgcagatttcaaccgggcttg

SEQ ID NO: 555 - RNA, 88 nt, synthetic construct
  source 1..88: mol_type = other RNA
acagcttatttggaagctgaaatgtgaggtttataacactcacaagaatcctgaaaaggatgccaaacctctggcagatttcaaccgg

SEQ ID NO: 556 - RNA, 88 nt, synthetic construct
  source 1..88: mol_type = other RNA
acagcttatttggaagctgaaatgtgaggtttataacactcacaagaatcctgaaaaggatgccaaacaaatctgccagagcaggtac

SEQ ID NO: 557 - RNA, 88 nt, synthetic construct
  source 1..88: mol_type = other RNA
acagcttatttggaagctgaaatgtgaggtttataacactcacaagaatcctgaaaaggatgccaaaccctgctctggcagatttcaa

SEQ ID NO: 558 - RNA, 88 nt, synthetic construct
  source 1..88: mol_type = other RNA
acagcttatttggaagctgaaatgtgaggtttataacactcacaagaatcctgaaaaggatgccaaacccagagcaggtacctccaac

SEQ ID NO: 559 - RNA, 88 nt, synthetic construct
  source 1..88: mol_type = other RNA
acagcttatttggaagctgaaatgtgaggtttataacactcacaagaatcctgaaaaggatgccaaacgaggtacctgctctggcaga

SEQ ID NO: 560 - RNA, 88 nt, synthetic construct
  source 1..88: mol_type = other RNA
acagcttatttggaagctgaaatgtgaggtttataacactcacaagaatcctgaaaaggatgccaaacttggaggtacctgctctggc

SEQ ID NO: 561 - RNA, 88 nt, synthetic construct
  source 1..88: mol_type = other RNA
acagcttatttggaagctgaaatgtgaggtttataacactcacaagaatcctgaaaaggatgccaaacatgttggaggtacctgctct

SEQ ID NO: 562 - RNA, 88 nt, synthetic construct
  source 1..88: mol_type = other RNA
acagcttatttggaagctgaaatgtgaggtttataacactcacaagaatcctgaaaaggatgccaaaccctccaacatcaaggaagat

SEQ ID NO: 563 - RNA, 88 nt, synthetic construct
  source 1..88: mol_type = other RNA
acagcttatttggaagctgaaatgtgaggtttataacactcacaagaatcctgaaaaggatgccaaacccatcttccttgatgttgga

SEQ ID NO: 564 - RNA, 88 nt, synthetic construct
  source 1..88: mol_type = other RNA
acagcttatttggaagctgaaatgtgaggtttataacactcacaagaatcctgaaaaggatgccaaacgaaatgccatcttccttgat

SEQ ID NO: 565 - RNA, 88 nt, synthetic construct
  source 1..88: mol_type = other RNA
acagcttatttggaagctgaaatgtgaggtttataacactcacaagaatcctgaaaaggatgccaaacgcatttctagtttggagatg

SEQ ID NO: 566 - RNA, 88 nt, synthetic construct
  source 1..88: mol_type = other RNA
acagcttatttggaagctgaaatgtgaggtttataacactcacaagaatcctgaaaaggatgccaaacccatctccaaactagaaatg

SEQ ID NO: 567 - RNA, 88 nt, synthetic construct
  source 1..88: mol_type = other RNA
acagcttatttggaagctgaaatgtgaggtttataacactcacaagaatcctgaaaaggatgccaaacgtttggagatggcagtttcc

SEQ ID NO: 568 - RNA, 88 nt, synthetic construct
  source 1..88: mol_type = other RNA
acagcttatttggaagctgaaatgtgaggtttataacactcacaagaatcctgaaaaggatgccaaacgagatggcagtttccttagt

SEQ ID NO: 569 - RNA, 88 nt, synthetic construct
  source 1..88: mol_type = other RNA
acagcttatttggaagctgaaatgtgaggtttataacactcacaagaatcctgaaaaggatgccaaacaggaaactgccatctccaaa

SEQ ID NO: 570 - RNA, 88 nt, synthetic construct
  source 1..88: mol_type = other RNA
acagcttatttggaagctgaaatgtgaggtttataacactcacaagaatcctgaaaaggatgccaaacctaaggaaactgccatctcc

SEQ ID NO: 571 - RNA, 88 nt, synthetic construct
  source 1..88: mol_type = other RNA
acagcttatttggaagctgaaatgtgaggtttataacactcacaagaatcctgaaaaggatgccaaacgcagtttccttagtaaccac

SEQ ID NO: 572 - RNA, 88 nt, synthetic construct
  source 1..88: mol_type = other RNA
acagcttatttggaagctgaaatgtgaggtttataacactcacaagaatcctgaaaaggatgccaaacgttactaaggaaactgccat

SEQ ID NO: 573 - RNA, 88 nt, synthetic construct
  source 1..88: mol_type = other RNA
acagcttatttggaagctgaaatgtgaggtttataacactcacaagaatcctgaaaaggatgccaaactggttactaaggaaactgcc

SEQ ID NO: 574 - RNA, 88 nt, synthetic construct
  source 1..88: mol_type = other RNA
acagcttatttggaagctgaaatgtgaggtttataacactcacaagaatcctgaaaaggatgccaaacgtaaccacaggttgtgtcac

SEQ ID NO: 575 - RNA, 88 nt, synthetic construct
  source 1..88: mol_type = other RNA
acagcttatttggaagctgaaatgtgaggtttataacactcacaagaatcctgaaaaggatgccaaacacacaacctgtggttactaa

SEQ ID NO: 576 - RNA, 88 nt, synthetic construct
  source 1..88: mol_type = other RNA
acagcttatttggaagctgaaatgtgaggtttataacactcacaagaatcctgaaaaggatgccaaacaccacaggttgtgtcaccag

SEQ ID NO: 577 - RNA, 88 nt, synthetic construct
  source 1..88: mol_type = other RNA
acagcttatttggaagctgaaatgtgaggtttataacactcacaagaatcctgaaaaggatgccaaacgtgacacaacctgtggttac

SEQ ID NO: 578 - RNA, 88 nt, synthetic construct
  source 1..88: mol_type = other RNA
acagcttatttggaagctgaaatgtgaggtttataacactcacaagaatcctgaaaaggatgccaaacctctggtgacacaacctgtg

SEQ ID NO: 579 - RNA, 88 nt, synthetic construct
  source 1..88: mol_type = other RNA
acagcttatttggaagctgaaatgtgaggtttataacactcacaagaatcctgaaaaggatgccaaacttactctggtgacacaacct

SEQ ID NO: 580 - RNA, 88 nt, synthetic construct
  source 1..88: mol_type = other RNA
acagcttatttggaagctgaaatgtgaggtttataacactcacaagaatcctgaaaaggatgccaaactgtcaccagagtaacagtct

SEQ ID NO: 581 - RNA, 88 nt, synthetic construct
  source 1..88: mol_type = other RNA
acagcttatttggaagctgaaatgtgaggtttataacactcacaagaatcctgaaaaggatgccaaactcaccagagtaacagtctga

SEQ ID NO: 582 - RNA, 88 nt, synthetic construct
  source 1..88: mol_type = other RNA
acagcttatttggaagctgaaatgtgaggtttataacactcacaagaatcctgaaaaggatgccaaacctcagactgttactctggtg

SEQ ID NO: 583 - RNA, 88 nt, synthetic construct
  source 1..88: mol_type = other RNA
acagcttatttggaagctgaaatgtgaggtttataacactcacaagaatcctgaaaaggatgccaaaccaaacagctgtcagacagaa

SEQ ID NO: 584 - RNA, 88 nt, synthetic construct
  source 1..88: mol_type = other RNA
acagcttatttggaagctgaaatgtgaggtttataacactcacaagaatcctgaaaaggatgccaaactctgacagctgtttgcagac

SEQ ID NO: 585 - RNA, 88 nt, synthetic construct
  source 1..88: mol_type = other RNA
acagcttatttggaagctgaaatgtgaggtttataacactcacaagaatcctgaaaaggatgccaaacacagctgtttgcagacctcc

SEQ ID NO: 586 - RNA, 88 nt, synthetic construct
  source 1..88: mol_type = other RNA
acagcttatttggaagctgaaatgtgaggtttataacactcacaagaatcctgaaaaggatgccaaactttgcagacctcctgccacc

SEQ ID NO: 587 - RNA, 88 nt, synthetic construct
  source 1..88: mol_type = other RNA
acagcttatttggaagctgaaatgtgaggtttataacactcacaagaatcctgaaaaggatgccaaacgcaggaggtctgcaaacagc

SEQ ID NO: 588 - RNA, 88 nt, synthetic construct
  source 1..88: mol_type = other RNA
acagcttatttggaagctgaaatgtgaggtttataacactcacaagaatcctgaaaaggatgccaaaccagacctcctgccaccgcag

SEQ ID NO: 589 - RNA, 88 nt, synthetic construct
  source 1..88: mol_type = other RNA
acagcttatttggaagctgaaatgtgaggtttataacactcacaagaatcctgaaaaggatgccaaaccggtggcaggaggtctgcaa

SEQ ID NO: 590 - RNA, 88 nt, synthetic construct
  source 1..88: mol_type = other RNA
acagcttatttggaagctgaaatgtgaggtttataacactcacaagaatcctgaaaaggatgccaaacaatctgcggtggcaggaggt

SEQ ID NO: 591 - RNA, 88 nt, synthetic construct
  source 1..88: mol_type = other RNA
acagcttatttggaagctgaaatgtgaggtttataacactcacaagaatcctgaaaaggatgccaaacccaccgcagattcaggcttc

SEQ ID NO: 592 - RNA, 88 nt, synthetic construct
  source 1..88: mol_type = other RNA
acagcttatttggaagctgaaatgtgaggtttataacactcacaagaatcctgaaaaggatgccaaacggaagcctgaatctgcggtg

SEQ ID NO: 593 - RNA, 88 nt, synthetic construct
  source 1..88: mol_type = other RNA
acagcttatttggaagctgaaatgtgaggtttataacactcacaagaatcctgaaaaggatgccaaaccaggaaaaattgggaagcct

SEQ ID NO: 594 - RNA, 88 nt, synthetic construct
  source 1..88: mol_type = other RNA
acagcttatttggaagctgaaatgtgaggtttataacactcacaagaatcctgaaaaggatgccaaacttctacaggaaaaattggga

SEQ ID NO: 595 - RNA, 88 nt, synthetic construct
  source 1..88: mol_type = other RNA
acagcttatttggaagctgaaatgtgaggtttataacactcacaagaatcctgaaaaggatgccaaacccagtattctacaggaaaaa

SEQ ID NO: 596 - RNA, 88 nt, synthetic construct
  source 1..88: mol_type = other RNA
acagcttatttggaagctgaaatgtgaggtttataacactcacaagaatcctgaaaaggatgccaaactagaatactggcatctgttt

SEQ ID NO: 597 - RNA, 88 nt, synthetic construct
  source 1..88: mol_type = other RNA
acagcttatttggaagctgaaatgtgaggtttataacactcacaagaatcctgaaaaggatgccaaacgaatactggcatctgttttt

SEQ ID NO: 598 - RNA, 88 nt, synthetic construct
  source 1..88: mol_type = other RNA
acagcttatttggaagctgaaatgtgaggtttataacactcacaagaatcctgaaaaggatgccaaacctggcatctgtttttgagga

SEQ ID NO: 599 - RNA, 88 nt, synthetic construct
  source 1..88: mol_type = other RNA
acagcttatttggaagctgaaatgtgaggtttataacactcacaagaatcctgaaaaggatgccaaacgcatctgtttttgaggattg

SEQ ID NO: 600 - RNA, 88 nt, synthetic construct
  source 1..88: mol_type = other RNA
acagcttatttggaagctgaaatgtgaggtttataacactcacaagaatcctgaaaaggatgccaaactttttgaggattgctgaatt

SEQ ID NO: 601 - RNA, 88 nt, synthetic construct
  source 1..88: mol_type = other RNA
acagcttatttggaagctgaaatgtgaggtttataacactcacaagaatcctgaaaaggatgccaaacattcagcaatcctcaaaaac

SEQ ID NO: 602 - RNA, 88 nt, synthetic construct
  source 1..88: mol_type = other RNA
acagcttatttggaagctgaaatgtgaggtttataacactcacaagaatcctgaaaaggatgccaaacaggattgctgaattatttct

SEQ ID NO: 603 - RNA, 88 nt, synthetic construct
  source 1..88: mol_type = other RNA
acagcttatttggaagctgaaatgtgaggtttataacactcacaagaatcctgaaaaggatgccaaacctgaattatttcttccccag

SEQ ID NO: 604 - RNA, 88 nt, synthetic construct
  source 1..88: mol_type = other RNA
acagcttatttggaagctgaaatgtgaggtttataacactcacaagaatcctgaaaaggatgccaaacgggaagaaataattcagcaa

SEQ ID NO: 605 - RNA, 88 nt, synthetic construct
  source 1..88: mol_type = other RNA
acagcttatttggaagctgaaatgtgaggtttataacactcacaagaatcctgaaaaggatgccaaacaattatttcttccccagttg

SEQ ID NO: 606 - RNA, 88 nt, synthetic construct
  source 1..88: mol_type = other RNA
acagcttatttggaagctgaaatgtgaggtttataacactcacaagaatcctgaaaaggatgccaaaccaactggggaagaaataatt

SEQ ID NO: 607 - RNA, 88 nt, synthetic construct
  source 1..88: mol_type = other RNA
acagcttatttggaagctgaaatgtgaggtttataacactcacaagaatcctgaaaaggatgccaaactttcttccccagttgcattc

SEQ ID NO: 608 - RNA, 88 nt, synthetic construct
  source 1..88: mol_type = other RNA
acagcttatttggaagctgaaatgtgaggtttataacactcacaagaatcctgaaaaggatgccaaacaatgcaactggggaagaaat

SEQ ID NO: 609 - RNA, 88 nt, synthetic construct
  source 1..88: mol_type = other RNA
acagcttatttggaagctgaaatgtgaggtttataacactcacaagaatcctgaaaaggatgccaaactcagaacattgaatgcaact

SEQ ID NO: 610 - RNA, 88 nt, synthetic construct
  source 1..88: mol_type = other RNA
acagcttatttggaagctgaaatgtgaggtttataacactcacaagaatcctgaaaaggatgccaaaccattcaatgttctgacaaca

SEQ ID NO: 611 - RNA, 88 nt, synthetic construct
  source 1..88: mol_type = other RNA
acagcttatttggaagctgaaatgtgaggtttataacactcacaagaatcctgaaaaggatgccaaacttgtcagaacattgaatgca

SEQ ID NO: 612 - RNA, 88 nt, synthetic construct
  source 1..88: mol_type = other RNA
acagcttatttggaagctgaaatgtgaggtttataacactcacaagaatcctgaaaaggatgccaaacttctgacaacagtttgccgc

SEQ ID NO: 613 - RNA, 88 nt, synthetic construct
  source 1..88: mol_type = other RNA
acagcttatttggaagctgaaatgtgaggtttataacactcacaagaatcctgaaaaggatgccaaacacaacagtttgccgctgccc

SEQ ID NO: 614 - RNA, 88 nt, synthetic construct
  source 1..88: mol_type = other RNA
acagcttatttggaagctgaaatgtgaggtttataacactcacaagaatcctgaaaaggatgccaaacggcagcggcaaactgttgtc

SEQ ID NO: 615 - RNA, 88 nt, synthetic construct
  source 1..88: mol_type = other RNA
acagcttatttggaagctgaaatgtgaggtttataacactcacaagaatcctgaaaaggatgccaaacgcattgggcagcggcaaact

SEQ ID NO: 616 - RNA, 88 nt, synthetic construct
  source 1..88: mol_type = other RNA
acagcttatttggaagctgaaatgtgaggtttataacactcacaagaatcctgaaaaggatgccaaacccgctgcccaatgccatcct

SEQ ID NO: 617 - RNA, 88 nt, synthetic construct
  source 1..88: mol_type = other RNA
acagcttatttggaagctgaaatgtgaggtttataacactcacaagaatcctgaaaaggatgccaaaccccaatgccatcctggagtt

SEQ ID NO: 618 - RNA, 88 nt, synthetic construct
  source 1..88: mol_type = other RNA
acagcttatttggaagctgaaatgtgaggtttataacactcacaagaatcctgaaaaggatgccaaacagataccatttgtatttagc

SEQ ID NO: 619 - RNA, 88 nt, synthetic construct
  source 1..88: mol_type = other RNA
acagcttatttggaagctgaaatgtgaggtttataacactcacaagaatcctgaaaaggatgccaaacaatacaaatggtatcttaag

SEQ ID NO: 620 - RNA, 88 nt, synthetic construct
  source 1..88: mol_type = other RNA
acagcttatttggaagctgaaatgtgaggtttataacactcacaagaatcctgaaaaggatgccaaacctaaatacaaatggtatctt

SEQ ID NO: 621 - RNA, 88 nt, synthetic construct
  source 1..88: mol_type = other RNA
acagcttatttggaagctgaaatgtgaggtttataacactcacaagaatcctgaaaaggatgccaaacccatttgtatttagcatgtt

SEQ ID NO: 622 - RNA, 88 nt, synthetic construct
  source 1..88: mol_type = other RNA
acagcttatttggaagctgaaatgtgaggtttataacactcacaagaatcctgaaaaggatgccaaacggaacatgctaaatacaaat

SEQ ID NO: 623 - RNA, 88 nt, synthetic construct
  source 1..88: mol_type = other RNA
acagcttatttggaagctgaaatgtgaggtttataacactcacaagaatcctgaaaaggatgccaaactatttagcatgttcccaatt

SEQ ID NO: 624 - RNA, 88 nt, synthetic construct
  source 1..88: mol_type = other RNA
acagcttatttggaagctgaaatgtgaggtttataacactcacaagaatcctgaaaaggatgccaaactttagcatgttcccaattct

SEQ ID NO: 625 - RNA, 88 nt, synthetic construct
  source 1..88: mol_type = other RNA
acagcttatttggaagctgaaatgtgaggtttataacactcacaagaatcctgaaaaggatgccaaacgcatgttcccaattctcagg

SEQ ID NO: 626 - RNA, 88 nt, synthetic construct
  source 1..88: mol_type = other RNA
acagcttatttggaagctgaaatgtgaggtttataacactcacaagaatcctgaaaaggatgccaaacagaattgggaacatgctaaa

SEQ ID NO: 627 - RNA, 88 nt, synthetic construct
  source 1..88: mol_type = other RNA
acagcttatttggaagctgaaatgtgaggtttataacactcacaagaatcctgaaaaggatgccaaacttcccaattctcaggaattt

SEQ ID NO: 628 - RNA, 88 nt, synthetic construct
  source 1..88: mol_type = other RNA
acagcttatttggaagctgaaatgtgaggtttataacactcacaagaatcctgaaaaggatgccaaactgtctttctgagaaactgtt

SEQ ID NO: 629 - RNA, 88 nt, synthetic construct
  source 1..88: mol_type = other RNA
acagcttatttggaagctgaaatgtgaggtttataacactcacaagaatcctgaaaaggatgccaaactctttctgagaaactgttca

SEQ ID NO: 630 - RNA, 88 nt, synthetic construct
  source 1..88: mol_type = other RNA
acagcttatttggaagctgaaatgtgaggtttataacactcacaagaatcctgaaaaggatgccaaacaacagtttctcagaaagaca

SEQ ID NO: 631 - RNA, 88 nt, synthetic construct
  source 1..88: mol_type = other RNA
acagcttatttggaagctgaaatgtgaggtttataacactcacaagaatcctgaaaaggatgccaaacagaaactgttcagcttctgt

SEQ ID NO: 632 - RNA, 88 nt, synthetic construct
  source 1..88: mol_type = other RNA
acagcttatttggaagctgaaatgtgaggtttataacactcacaagaatcctgaaaaggatgccaaacacagaagctgaacagtttct

SEQ ID NO: 633 - RNA, 88 nt, synthetic construct
  source 1..88: mol_type = other RNA
acagcttatttggaagctgaaatgtgaggtttataacactcacaagaatcctgaaaaggatgccaaacttcagcttctgttagccact

SEQ ID NO: 634 - RNA, 88 nt, synthetic construct
  source 1..88: mol_type = other RNA
acagcttatttggaagctgaaatgtgaggtttataacactcacaagaatcctgaaaaggatgccaaacgctaacagaagctgaacagt

SEQ ID NO: 635 - RNA, 88 nt, synthetic construct
  source 1..88: mol_type = other RNA
acagcttatttggaagctgaaatgtgaggtttataacactcacaagaatcctgaaaaggatgccaaacatcagtggctaacagaagct

SEQ ID NO: 636 - RNA, 88 nt, synthetic construct
  source 1..88: mol_type = other RNA
acagcttatttggaagctgaaatgtgaggtttataacactcacaagaatcctgaaaaggatgccaaacttagccactgattaaatatc

SEQ ID NO: 637 - RNA, 88 nt, synthetic construct
  source 1..88: mol_type = other RNA
acagcttatttggaagctgaaatgtgaggtttataacactcacaagaatcctgaaaaggatgccaaactttaatcagtggctaacaga

SEQ ID NO: 638 - RNA, 88 nt, synthetic construct
  source 1..88: mol_type = other RNA
acagcttatttggaagctgaaatgtgaggtttataacactcacaagaatcctgaaaaggatgccaaacgccactgattaaatatcttt

SEQ ID NO: 639 - RNA, 88 nt, synthetic construct
  source 1..88: mol_type = other RNA
acagcttatttggaagctgaaatgtgaggtttataacactcacaagaatcctgaaaaggatgccaaacaagatatttaatcagtggct

SEQ ID NO: 640 - RNA, 88 nt, synthetic construct
  source 1..88: mol_type = other RNA
acagcttatttggaagctgaaatgtgaggtttataacactcacaagaatcctgaaaaggatgccaaactaaagatatttaatcagtgg

SEQ ID NO: 641 - RNA, 88 nt, synthetic construct
  source 1..88: mol_type = other RNA
acagcttatttggaagctgaaatgtgaggtttataacactcacaagaatcctgaaaaggatgccaaactatcataatgaaaacgccgc

SEQ ID NO: 642 - RNA, 88 nt, synthetic construct
  source 1..88: mol_type = other RNA
acagcttatttggaagctgaaatgtgaggtttataacactcacaagaatcctgaaaaggatgccaaactcataatgaaaacgccgcca

SEQ ID NO: 643 - RNA, 88 nt, synthetic construct
  source 1..88: mol_type = other RNA
acagcttatttggaagctgaaatgtgaggtttataacactcacaagaatcctgaaaaggatgccaaacgcggcgttttcattatgata

SEQ ID NO: 644 - RNA, 88 nt, synthetic construct
  source 1..88: mol_type = other RNA
acagcttatttggaagctgaaatgtgaggtttataacactcacaagaatcctgaaaaggatgccaaacatgaaaacgccgccatttct

SEQ ID NO: 645 - RNA, 88 nt, synthetic construct
  source 1..88: mol_type = other RNA
acagcttatttggaagctgaaatgtgaggtttataacactcacaagaatcctgaaaaggatgccaaacaaaacgccgccatttctcaa

SEQ ID NO: 646 - RNA, 88 nt, synthetic construct
  source 1..88: mol_type = other RNA
acagcttatttggaagctgaaatgtgaggtttataacactcacaagaatcctgaaaaggatgccaaacagaaatggcggcgttttcat

SEQ ID NO: 647 - RNA, 88 nt, synthetic construct
  source 1..88: mol_type = other RNA
acagcttatttggaagctgaaatgtgaggtttataacactcacaagaatcctgaaaaggatgccaaacttgagaaatggcggcgtttt

SEQ ID NO: 648 - RNA, 88 nt, synthetic construct
  source 1..88: mol_type = other RNA
acagcttatttggaagctgaaatgtgaggtttataacactcacaagaatcctgaaaaggatgccaaacacagatctgttgagaaatgg

SEQ ID NO: 649 - RNA, 89 nt, synthetic construct
  source 1..89: mol_type = other RNA
gacagcttatttggaagctgaaatgtgaggtttataacactcacaagaatcctgaaaaggatgccaaacgtttctgtgattttcttttg

SEQ ID NO: 650 - RNA, 89 nt, synthetic construct
  source 1..89: mol_type = other RNA
gacagcttatttggaagctgaaatgtgaggtttataacactcacaagaatcctgaaaaggatgccaaactgattttcttttggattgca

SEQ ID NO: 651 - RNA, 89 nt, synthetic construct
  source 1..89: mol_type = other RNA
gacagcttatttggaagctgaaatgtgaggtttataacactcacaagaatcctgaaaaggatgccaaacattttcttttggattgcatc

SEQ ID NO: 652 - RNA, 89 nt, synthetic construct
  source 1..89: mol_type = other RNA
gacagcttatttggaagctgaaatgtgaggtttataacactcacaagaatcctgaaaaggatgccaaaccaatccaaaagaaaatcaca

SEQ ID NO: 653 - RNA, 89 nt, synthetic construct
  source 1..89: mol_type = other RNA
gacagcttatttggaagctgaaatgtgaggtttataacactcacaagaatcctgaaaaggatgccaaacgatgcaatccaaaagaaaat

SEQ ID NO: 654 - RNA, 89 nt, synthetic construct
  source 1..89: mol_type = other RNA
gacagcttatttggaagctgaaatgtgaggtttataacactcacaagaatcctgaaaaggatgccaaaccagtagatgcaatccaaaag

SEQ ID NO: 655 - RNA, 89 nt, synthetic construct
  source 1..89: mol_type = other RNA
gacagcttatttggaagctgaaatgtgaggtttataacactcacaagaatcctgaaaaggatgccaaacgattgcatctactgtatagg

SEQ ID NO: 656 - RNA, 89 nt, synthetic construct
  source 1..89: mol_type = other RNA
gacagcttatttggaagctgaaatgtgaggtttataacactcacaagaatcctgaaaaggatgccaaactacagtagatgcaatccaaa

SEQ ID NO: 657 - RNA, 89 nt, synthetic construct
  source 1..89: mol_type = other RNA
gacagcttatttggaagctgaaatgtgaggtttataacactcacaagaatcctgaaaaggatgccaaaccatctactgtatagggaccc

SEQ ID NO: 658 - RNA, 89 nt, synthetic construct
  source 1..89: mol_type = other RNA
gacagcttatttggaagctgaaatgtgaggtttataacactcacaagaatcctgaaaaggatgccaaacctgtatagggaccctccttc

SEQ ID NO: 659 - RNA, 89 nt, synthetic construct
  source 1..89: mol_type = other RNA
gacagcttatttggaagctgaaatgtgaggtttataacactcacaagaatcctgaaaaggatgccaaactatagggaccctccttccat

SEQ ID NO: 660 - RNA, 89 nt, synthetic construct
  source 1..89: mol_type = other RNA
gacagcttatttggaagctgaaatgtgaggtttataacactcacaagaatcctgaaaaggatgccaaacgaaggagggtccctatacag

SEQ ID NO: 661 - RNA, 89 nt, synthetic construct
  source 1..89: mol_type = other RNA
gacagcttatttggaagctgaaatgtgaggtttataacactcacaagaatcctgaaaaggatgccaaactagggaccctccttccatga

SEQ ID NO: 662 - RNA, 89 nt, synthetic construct
  source 1..89: mol_type = other RNA
gacagcttatttggaagctgaaatgtgaggtttataacactcacaagaatcctgaaaaggatgccaaacgggaccctccttccatgact

SEQ ID NO: 663 - RNA, 89 nt, synthetic construct
  source 1..89: mol_type = other RNA
gacagcttatttggaagctgaaatgtgaggtttataacactcacaagaatcctgaaaaggatgccaaacagtcatggaaggagggtccc

SEQ ID NO: 664 - RNA, 89 nt, synthetic construct
  source 1..89: mol_type = other RNA
gacagcttatttggaagctgaaatgtgaggtttataacactcacaagaatcctgaaaaggatgccaaacactcaagcttggctctggcc

SEQ ID NO: 665 - RNA, 89 nt, synthetic construct
  source 1..89: mol_type = other RNA
gacagcttatttggaagctgaaatgtgaggtttataacactcacaagaatcctgaaaaggatgccaaacggacaggccagagccaagct

SEQ ID NO: 666 - RNA, 89 nt, synthetic construct
  source 1..89: mol_type = other RNA
gacagcttatttggaagctgaaatgtgaggtttataacactcacaagaatcctgaaaaggatgccaaacgctctggcctgtcctaagac

SEQ ID NO: 667 - RNA, 89 nt, synthetic construct
  source 1..89: mol_type = other RNA
gacagcttatttggaagctgaaatgtgaggtttataacactcacaagaatcctgaaaaggatgccaaacgcctgtcctaagacctgctc

SEQ ID NO: 668 - RNA, 89 nt, synthetic construct
  source 1..89: mol_type = other RNA
gacagcttatttggaagctgaaatgtgaggtttataacactcacaagaatcctgaaaaggatgccaaacagcaggtcttaggacaggcc

SEQ ID NO: 669 - RNA, 89 nt, synthetic construct
  source 1..89: mol_type = other RNA
gacagcttatttggaagctgaaatgtgaggtttataacactcacaagaatcctgaaaaggatgccaaactcctaagacctgctcagctt

SEQ ID NO: 670 - RNA, 89 nt, synthetic construct
  source 1..89: mol_type = other RNA
gacagcttatttggaagctgaaatgtgaggtttataacactcacaagaatcctgaaaaggatgccaaacagacctgctcagcttcttcc

SEQ ID NO: 671 - RNA, 89 nt, synthetic construct
  source 1..89: mol_type = other RNA
gacagcttatttggaagctgaaatgtgaggtttataacactcacaagaatcctgaaaaggatgccaaacaggaagaagctgagcaggtc

SEQ ID NO: 672 - RNA, 89 nt, synthetic construct
  source 1..89: mol_type = other RNA
gacagcttatttggaagctgaaatgtgaggtttataacactcacaagaatcctgaaaaggatgccaaacctcagcttcttccttagctt

SEQ ID NO: 673 - RNA, 89 nt, synthetic construct
  source 1..89: mol_type = other RNA
gacagcttatttggaagctgaaatgtgaggtttataacactcacaagaatcctgaaaaggatgccaaacgaagctaaggaagaagctga

SEQ ID NO: 674 - RNA, 89 nt, synthetic construct
  source 1..89: mol_type = other RNA
gacagcttatttggaagctgaaatgtgaggtttataacactcacaagaatcctgaaaaggatgccaaacgctggaagctaaggaagaag

SEQ ID NO: 675 - RNA, 89 nt, synthetic construct
  source 1..89: mol_type = other RNA
gacagcttatttggaagctgaaatgtgaggtttataacactcacaagaatcctgaaaaggatgccaaacgcttccagccattgtgttga

SEQ ID NO: 676 - RNA, 89 nt, synthetic construct
  source 1..89: mol_type = other RNA
gacagcttatttggaagctgaaatgtgaggtttataacactcacaagaatcctgaaaaggatgccaaacaaggattcaacacaatggct

SEQ ID NO: 677 - RNA, 89 nt, synthetic construct
  source 1..89: mol_type = other RNA
gacagcttatttggaagctgaaatgtgaggtttataacactcacaagaatcctgaaaaggatgccaaacttaaaggattcaacacaatg

SEQ ID NO: 678 - RNA, 89 nt, synthetic construct
  source 1..89: mol_type = other RNA
gacagcttatttggaagctgaaatgtgaggtttataacactcacaagaatcctgaaaaggatgccaaactgttgaatcctttaacattt

SEQ ID NO: 679 - RNA, 89 nt, synthetic construct
  source 1..89: mol_type = other RNA
gacagcttatttggaagctgaaatgtgaggtttataacactcacaagaatcctgaaaaggatgccaaacttgaatcctttaacatttca

SEQ ID NO: 680 - RNA, 89 nt, synthetic construct
  source 1..89: mol_type = other RNA
gacagcttatttggaagctgaaatgtgaggtttataacactcacaagaatcctgaaaaggatgccaaacaaatgttaaaggattcaaca

SEQ ID NO: 681 - RNA, 89 nt, synthetic construct
  source 1..89: mol_type = other RNA
gacagcttatttggaagctgaaatgtgaggtttataacactcacaagaatcctgaaaaggatgccaaacaatcctttaacatttcattc

SEQ ID NO: 682 - RNA, 89 nt, synthetic construct
  source 1..89: mol_type = other RNA
gacagcttatttggaagctgaaatgtgaggtttataacactcacaagaatcctgaaaaggatgccaaacaatgaaatgttaaaggattc

SEQ ID NO: 683 - RNA, 89 nt, synthetic construct
  source 1..89: mol_type = other RNA
gacagcttatttggaagctgaaatgtgaggtttataacactcacaagaatcctgaaaaggatgccaaacacatttcattcaactgttgc

SEQ ID NO: 684 - RNA, 89 nt, synthetic construct
  source 1..89: mol_type = other RNA
gacagcttatttggaagctgaaatgtgaggtttataacactcacaagaatcctgaaaaggatgccaaacttgcctccggttctgaaggt

SEQ ID NO: 685 - RNA, 89 nt, synthetic construct
  source 1..89: mol_type = other RNA
gacagcttatttggaagctgaaatgtgaggtttataacactcacaagaatcctgaaaaggatgccaaaccctccggttctgaaggtgtt

SEQ ID NO: 686 - RNA, 89 nt, synthetic construct
  source 1..89: mol_type = other RNA
gacagcttatttggaagctgaaatgtgaggtttataacactcacaagaatcctgaaaaggatgccaaaccaagaacaccttcagaaccg

SEQ ID NO: 687 - RNA, 89 nt, synthetic construct
  source 1..89: mol_type = other RNA
gacagcttatttggaagctgaaatgtgaggtttataacactcacaagaatcctgaaaaggatgccaaacaaggtgttcttgtacttcat

SEQ ID NO: 688 - RNA, 89 nt, synthetic construct
  source 1..89: mol_type = other RNA
gacagcttatttggaagctgaaatgtgaggtttataacactcacaagaatcctgaaaaggatgccaaacaagtacaagaacaccttcag

SEQ ID NO: 689 - RNA, 89 nt, synthetic construct
  source 1..89: mol_type = other RNA
gacagcttatttggaagctgaaatgtgaggtttataacactcacaagaatcctgaaaaggatgccaaacttcttgtacttcatcccact

SEQ ID NO: 690 - RNA, 89 nt, synthetic construct
  source 1..89: mol_type = other RNA
gacagcttatttggaagctgaaatgtgaggtttataacactcacaagaatcctgaaaaggatgccaaacggatgaagtacaagaacacc

SEQ ID NO: 691 - RNA, 89 nt, synthetic construct
  source 1..89: mol_type = other RNA
gacagcttatttggaagctgaaatgtgaggtttataacactcacaagaatcctgaaaaggatgccaaactacttcatcccactgattct

SEQ ID NO: 692 - RNA, 89 nt, synthetic construct
  source 1..89: mol_type = other RNA
gacagcttatttggaagctgaaatgtgaggtttataacactcacaagaatcctgaaaaggatgccaaaccttcatcccactgattctga

SEQ ID NO: 693 - RNA, 89 nt, synthetic construct
  source 1..89: mol_type = other RNA
gacagcttatttggaagctgaaatgtgaggtttataacactcacaagaatcctgaaaaggatgccaaacgaacaatcattacggatcga

SEQ ID NO: 694 - RNA, 89 nt, synthetic construct
  source 1..89: mol_type = other RNA
gacagcttatttggaagctgaaatgtgaggtttataacactcacaagaatcctgaaaaggatgccaaacatgattgttctagcctcttg

SEQ ID NO: 695 - RNA, 89 nt, synthetic construct
  source 1..89: mol_type = other RNA
gacagcttatttggaagctgaaatgtgaggtttataacactcacaagaatcctgaaaaggatgccaaacattgttctagcctcttgatt

SEQ ID NO: 696 - RNA, 89 nt, synthetic construct
  source 1..89: mol_type = other RNA
gacagcttatttggaagctgaaatgtgaggtttataacactcacaagaatcctgaaaaggatgccaaacttctagcctcttgattgctg

SEQ ID NO: 697 - RNA, 89 nt, synthetic construct
  source 1..89: mol_type = other RNA
gacagcttatttggaagctgaaatgtgaggtttataacactcacaagaatcctgaaaaggatgccaaacgcctcttgattgctggtctt

SEQ ID NO: 698 - RNA, 89 nt, synthetic construct
  source 1..89: mol_type = other RNA
gacagcttatttggaagctgaaatgtgaggtttataacactcacaagaatcctgaaaaggatgccaaacattgctggtcttgtttttca

SEQ ID NO: 699 - RNA, 89 nt, synthetic construct
  source 1..89: mol_type = other RNA
gacagcttatttggaagctgaaatgtgaggtttataacactcacaagaatcctgaaaaggatgccaaacaaaaacaagaccagcaatca

SEQ ID NO: 700 - RNA, 89 nt, synthetic construct
  source 1..89: mol_type = other RNA
gacagcttatttggaagctgaaatgtgaggtttataacactcacaagaatcctgaaaaggatgccaaacctggtcttgtttttcaaatt

SEQ ID NO: 701 - RNA, 89 nt, synthetic construct
  source 1..89: mol_type = other RNA
gacagcttatttggaagctgaaatgtgaggtttataacactcacaagaatcctgaaaaggatgccaaacgtcttgtttttcaaattttg

SEQ ID NO: 702 - RNA, 89 nt, synthetic construct
  source 1..89: mol_type = other RNA
gacagcttatttggaagctgaaatgtgaggtttataacactcacaagaatcctgaaaaggatgccaaactttttcaaattttgggcagc

SEQ ID NO: 703 - RNA, 89 nt, synthetic construct
  source 1..89: mol_type = other RNA
gacagcttatttggaagctgaaatgtgaggtttataacactcacaagaatcctgaaaaggatgccaaaccccaaaatttgaaaaacaag

SEQ ID NO: 704 - RNA, 89 nt, synthetic construct
  source 1..89: mol_type = other RNA
gacagcttatttggaagctgaaatgtgaggtttataacactcacaagaatcctgaaaaggatgccaaacccgctgcccaaaatttgaaa

SEQ ID NO: 705 - RNA, 89 nt, synthetic construct
  source 1..89: mol_type = other RNA
gacagcttatttggaagctgaaatgtgaggtttataacactcacaagaatcctgaaaaggatgccaaacggcagcggtaatgagttctt

SEQ ID NO: 706 - RNA, 89 nt, synthetic construct
  source 1..89: mol_type = other RNA
gacagcttatttggaagctgaaatgtgaggtttataacactcacaagaatcctgaaaaggatgccaaacgaagaactcattaccgctgc

SEQ ID NO: 707 - RNA, 89 nt, synthetic construct
  source 1..89: mol_type = other RNA
gacagcttatttggaagctgaaatgtgaggtttataacactcacaagaatcctgaaaaggatgccaaacatgagttcttccaactgggg

SEQ ID NO: 708 - RNA, 89 nt, synthetic construct
  source 1..89: mol_type = other RNA
gacagcttatttggaagctgaaatgtgaggtttataacactcacaagaatcctgaaaaggatgccaaacagttcttccaactggggacg

SEQ ID NO: 709 - RNA, 89 nt, synthetic construct
  source 1..89: mol_type = other RNA
gacagcttatttggaagctgaaatgtgaggtttataacactcacaagaatcctgaaaaggatgccaaacgaacagaggcgtccccagtt

SEQ ID NO: 710 - RNA, 89 nt, synthetic construct
  source 1..89: mol_type = other RNA
gacagcttatttggaagctgaaatgtgaggtttataacactcacaagaatcctgaaaaggatgccaaacgggacgcctctgttccaaat

SEQ ID NO: 711 - RNA, 89 nt, synthetic construct
  source 1..89: mol_type = other RNA
gacagcttatttggaagctgaaatgtgaggtttataacactcacaagaatcctgaaaaggatgccaaaccaggatttggaacagaggcg

SEQ ID NO: 712 - RNA, 89 nt, synthetic construct
  source 1..89: mol_type = other RNA
gacagcttatttggaagctgaaatgtgaggtttataacactcacaagaatcctgaaaaggatgccaaacttccaaatcctgcattgttg

SEQ ID NO: 713 - RNA, 89 nt, synthetic construct
  source 1..89: mol_type = other RNA
gacagcttatttggaagctgaaatgtgaggtttataacactcacaagaatcctgaaaaggatgccaaaccttgatgatcatctcgttga

SEQ ID NO: 714 - RNA, 89 nt, synthetic construct
  source 1..89: mol_type = other RNA
gacagcttatttggaagctgaaatgtgaggtttataacactcacaagaatcctgaaaaggatgccaaacatgatcatctcgttgatatc

SEQ ID NO: 715 - RNA, 89 nt, synthetic construct
  source 1..89: mol_type = other RNA
gacagcttatttggaagctgaaatgtgaggtttataacactcacaagaatcctgaaaaggatgccaaactcaacgagatgatcatcaag

SEQ ID NO: 716 - RNA, 89 nt, synthetic construct
  source 1..89: mol_type = other RNA
gacagcttatttggaagctgaaatgtgaggtttataacactcacaagaatcctgaaaaggatgccaaacatcatctcgttgatatcctc

SEQ ID NO: 717 - RNA, 89 nt, synthetic construct
  source 1..89: mol_type = other RNA
gacagcttatttggaagctgaaatgtgaggtttataacactcacaagaatcctgaaaaggatgccaaacaggatatcaacgagatgatc

SEQ ID NO: 718 - RNA, 89 nt, synthetic construct
  source 1..89: mol_type = other RNA
gacagcttatttggaagctgaaatgtgaggtttataacactcacaagaatcctgaaaaggatgccaaacaccttgaggatatcaacgag

SEQ ID NO: 719 - RNA, 89 nt, synthetic construct
  source 1..89: mol_type = other RNA
gacagcttatttggaagctgaaatgtgaggtttataacactcacaagaatcctgaaaaggatgccaaacatatcctcaaggtcacccac

SEQ ID NO: 720 - RNA, 89 nt, synthetic construct
  source 1..89: mol_type = other RNA
gacagcttatttggaagctgaaatgtgaggtttataacactcacaagaatcctgaaaaggatgccaaacggtgaccttgaggatatcaa

SEQ ID NO: 721 - RNA, 89 nt, synthetic construct
  source 1..89: mol_type = other RNA
gacagcttatttggaagctgaaatgtgaggtttataacactcacaagaatcctgaaaaggatgccaaactcctcaaggtcacccaccat

SEQ ID NO: 722 - RNA, 89 nt, synthetic construct
  source 1..89: mol_type = other RNA
gacagcttatttggaagctgaaatgtgaggtttataacactcacaagaatcctgaaaaggatgccaaacgtgggtgaccttgaggatat

SEQ ID NO: 723 - RNA, 89 nt, synthetic construct
  source 1..89: mol_type = other RNA
gacagcttatttggaagctgaaatgtgaggtttataacactcacaagaatcctgaaaaggatgccaaacatggtgggtgaccttgagga

SEQ ID NO: 724 - RNA, 89 nt, synthetic construct
  source 1..89: mol_type = other RNA
gacagcttatttggaagctgaaatgtgaggtttataacactcacaagaatcctgaaaaggatgccaaacaaatcacagagggtgatggt

SEQ ID NO: 725 - RNA, 89 nt, synthetic construct
  source 1..89: mol_type = other RNA
gacagcttatttggaagctgaaatgtgaggtttataacactcacaagaatcctgaaaaggatgccaaactaaaatcacagagggtgatg

SEQ ID NO: 726 - RNA, 89 nt, synthetic construct
  source 1..89: mol_type = other RNA
gacagcttatttggaagctgaaatgtgaggtttataacactcacaagaatcctgaaaaggatgccaaacatcaagttataaaatcacag

SEQ ID NO: 727 - RNA, 89 nt, synthetic construct
  source 1..89: mol_type = other RNA
gacagcttatttggaagctgaaatgtgaggtttataacactcacaagaatcctgaaaaggatgccaaacattttataacttgatcaagc

SEQ ID NO: 728 - RNA, 89 nt, synthetic construct
  source 1..89: mol_type = other RNA
gacagcttatttggaagctgaaatgtgaggtttataacactcacaagaatcctgaaaaggatgccaaaccttgatcaagttataaaatc

SEQ ID NO: 729 - RNA, 89 nt, synthetic construct
  source 1..89: mol_type = other RNA
gacagcttatttggaagctgaaatgtgaggtttataacactcacaagaatcctgaaaaggatgccaaactaacttgatcaagcagagaa

SEQ ID NO: 730 - RNA, 89 nt, synthetic construct
  source 1..89: mol_type = other RNA
gacagcttatttggaagctgaaatgtgaggtttataacactcacaagaatcctgaaaaggatgccaaacacttgatcaagcagagaaag

SEQ ID NO: 731 - RNA, 89 nt, synthetic construct
  source 1..89: mol_type = other RNA
gacagcttatttggaagctgaaatgtgaggtttataacactcacaagaatcctgaaaaggatgccaaacatcaagcagagaaagccagt

SEQ ID NO: 732 - RNA, 89 nt, synthetic construct
  source 1..89: mol_type = other RNA
gacagcttatttggaagctgaaatgtgaggtttataacactcacaagaatcctgaaaaggatgccaaacgctttctctgcttgatcaag

SEQ ID NO: 733 - RNA, 89 nt, synthetic construct
  source 1..89: mol_type = other RNA
gacagcttatttggaagctgaaatgtgaggtttataacactcacaagaatcctgaaaaggatgccaaacccgactggctttctctgctt

SEQ ID NO: 734 - RNA, 89 nt, synthetic construct
  source 1..89: mol_type = other RNA
gacagcttatttggaagctgaaatgtgaggtttataacactcacaagaatcctgaaaaggatgccaaacgacagaacttaccgactggc

SEQ ID NO: 735 - RNA, 89 nt, synthetic construct
  source 1..89: mol_type = other RNA
gacagcttatttggaagctgaaatgtgaggtttataacactcacaagaatcctgaaaaggatgccaaacagttctgtccaagcccggtt

SEQ ID NO: 736 - RNA, 89 nt, synthetic construct
  source 1..89: mol_type = other RNA
gacagcttatttggaagctgaaatgtgaggtttataacactcacaagaatcctgaaaaggatgccaaactccaagcccggttgaaatct

SEQ ID NO: 737 - RNA, 89 nt, synthetic construct
  source 1..89: mol_type = other RNA
gacagcttatttggaagctgaaatgtgaggtttataacactcacaagaatcctgaaaaggatgccaaacgcagatttcaaccgggcttg

SEQ ID NO: 738 - RNA, 89 nt, synthetic construct
  source 1..89: mol_type = other RNA
gacagcttatttggaagctgaaatgtgaggtttataacactcacaagaatcctgaaaaggatgccaaacctctggcagatttcaaccgg

SEQ ID NO: 739 - RNA, 89 nt, synthetic construct
  source 1..89: mol_type = other RNA
gacagcttatttggaagctgaaatgtgaggtttataacactcacaagaatcctgaaaaggatgccaaacaaatctgccagagcaggtac

SEQ ID NO: 740 - RNA, 89 nt, synthetic construct
  source 1..89: mol_type = other RNA
gacagcttatttggaagctgaaatgtgaggtttataacactcacaagaatcctgaaaaggatgccaaaccctgctctggcagatttcaa

SEQ ID NO: 741 - RNA, 89 nt, synthetic construct
  source 1..89: mol_type = other RNA
gacagcttatttggaagctgaaatgtgaggtttataacactcacaagaatcctgaaaaggatgccaaacccagagcaggtacctccaac

SEQ ID NO: 742 - RNA, 89 nt, synthetic construct
  source 1..89: mol_type = other RNA
gacagcttatttggaagctgaaatgtgaggtttataacactcacaagaatcctgaaaaggatgccaaacgaggtacctgctctggcaga

SEQ ID NO: 743 - RNA, 89 nt, synthetic construct
  source 1..89: mol_type = other RNA
gacagcttatttggaagctgaaatgtgaggtttataacactcacaagaatcctgaaaaggatgccaaacttggaggtacctgctctggc

SEQ ID NO: 744 - RNA, 89 nt, synthetic construct
  source 1..89: mol_type = other RNA
gacagcttatttggaagctgaaatgtgaggtttataacactcacaagaatcctgaaaaggatgccaaacatgttggaggtacctgctct

SEQ ID NO: 745 - RNA, 89 nt, synthetic construct
  source 1..89: mol_type = other RNA
gacagcttatttggaagctgaaatgtgaggtttataacactcacaagaatcctgaaaaggatgccaaaccctccaacatcaaggaagat

SEQ ID NO: 746 - RNA, 89 nt, synthetic construct
  source 1..89: mol_type = other RNA
gacagcttatttggaagctgaaatgtgaggtttataacactcacaagaatcctgaaaaggatgccaaacccatcttccttgatgttgga

SEQ ID NO: 747 - RNA, 89 nt, synthetic construct
  source 1..89: mol_type = other RNA
gacagcttatttggaagctgaaatgtgaggtttataacactcacaagaatcctgaaaaggatgccaaacgaaatgccatcttccttgat

SEQ ID NO: 748 - RNA, 89 nt, synthetic construct
  source 1..89: mol_type = other RNA
gacagcttatttggaagctgaaatgtgaggtttataacactcacaagaatcctgaaaaggatgccaaacgcatttctagtttggagatg

SEQ ID NO: 749 - RNA, 89 nt, synthetic construct
  source 1..89: mol_type = other RNA
gacagcttatttggaagctgaaatgtgaggtttataacactcacaagaatcctgaaaaggatgccaaacccatctccaaactagaaatg

SEQ ID NO: 750 - RNA, 89 nt, synthetic construct
  source 1..89: mol_type = other RNA
gacagcttatttggaagctgaaatgtgaggtttataacactcacaagaatcctgaaaaggatgccaaacgtttggagatggcagtttcc

SEQ ID NO: 751 - RNA, 89 nt, synthetic construct
  source 1..89: mol_type = other RNA
gacagcttatttggaagctgaaatgtgaggtttataacactcacaagaatcctgaaaaggatgccaaacgagatggcagtttccttagt

SEQ ID NO: 752 - RNA, 89 nt, synthetic construct
  source 1..89: mol_type = other RNA
gacagcttatttggaagctgaaatgtgaggtttataacactcacaagaatcctgaaaaggatgccaaacaggaaactgccatctccaaa

SEQ ID NO: 753 - RNA, 89 nt, synthetic construct
  source 1..89: mol_type = other RNA
gacagcttatttggaagctgaaatgtgaggtttataacactcacaagaatcctgaaaaggatgccaaacctaaggaaactgccatctcc

SEQ ID NO: 754 - RNA, 89 nt, synthetic construct
  source 1..89: mol_type = other RNA
gacagcttatttggaagctgaaatgtgaggtttataacactcacaagaatcctgaaaaggatgccaaacgcagtttccttagtaaccac

SEQ ID NO: 755 - RNA, 89 nt, synthetic construct
  source 1..89: mol_type = other RNA
gacagcttatttggaagctgaaatgtgaggtttataacactcacaagaatcctgaaaaggatgccaaacgttactaaggaaactgccat

SEQ ID NO: 756 - RNA, 89 nt, synthetic construct
  source 1..89: mol_type = other RNA
gacagcttatttggaagctgaaatgtgaggtttataacactcacaagaatcctgaaaaggatgccaaactggttactaaggaaactgcc

SEQ ID NO: 757 - RNA, 89 nt, synthetic construct
  source 1..89: mol_type = other RNA
gacagcttatttggaagctgaaatgtgaggtttataacactcacaagaatcctgaaaaggatgccaaacgtaaccacaggttgtgtcac

SEQ ID NO: 758 - RNA, 89 nt, synthetic construct
  source 1..89: mol_type = other RNA
gacagcttatttggaagctgaaatgtgaggtttataacactcacaagaatcctgaaaaggatgccaaacacacaacctgtggttactaa

SEQ ID NO: 759 - RNA, 89 nt, synthetic construct
  source 1..89: mol_type = other RNA
gacagcttatttggaagctgaaatgtgaggtttataacactcacaagaatcctgaaaaggatgccaaacaccacaggttgtgtcaccag

SEQ ID NO: 760 - RNA, 89 nt, synthetic construct
  source 1..89: mol_type = other RNA
gacagcttatttggaagctgaaatgtgaggtttataacactcacaagaatcctgaaaaggatgccaaacgtgacacaacctgtggttac

SEQ ID NO: 761 - RNA, 89 nt, synthetic construct
  source 1..89: mol_type = other RNA
gacagcttatttggaagctgaaatgtgaggtttataacactcacaagaatcctgaaaaggatgccaaacctctggtgacacaacctgtg

SEQ ID NO: 762 - RNA, 89 nt, synthetic construct
  source 1..89: mol_type = other RNA
gacagcttatttggaagctgaaatgtgaggtttataacactcacaagaatcctgaaaaggatgccaaacttactctggtgacacaacct

SEQ ID NO: 763 - RNA, 89 nt, synthetic construct
  source 1..89: mol_type = other RNA
gacagcttatttggaagctgaaatgtgaggtttataacactcacaagaatcctgaaaaggatgccaaactgtcaccagagtaacagtct

SEQ ID NO: 764 - RNA, 89 nt, synthetic construct
  source 1..89: mol_type = other RNA
gacagcttatttggaagctgaaatgtgaggtttataacactcacaagaatcctgaaaaggatgccaaactcaccagagtaacagtctga

SEQ ID NO: 765 - RNA, 89 nt, synthetic construct
  source 1..89: mol_type = other RNA
gacagcttatttggaagctgaaatgtgaggtttataacactcacaagaatcctgaaaaggatgccaaacctcagactgttactctggtg

SEQ ID NO: 766 - RNA, 89 nt, synthetic construct
  source 1..89: mol_type = other RNA
gacagcttatttggaagctgaaatgtgaggtttataacactcacaagaatcctgaaaaggatgccaaaccaaacagctgtcagacagaa

SEQ ID NO: 767 - RNA, 89 nt, synthetic construct
  source 1..89: mol_type = other RNA
gacagcttatttggaagctgaaatgtgaggtttataacactcacaagaatcctgaaaaggatgccaaactctgacagctgtttgcagac

SEQ ID NO: 768 - RNA, 89 nt, synthetic construct
  source 1..89: mol_type = other RNA
gacagcttatttggaagctgaaatgtgaggtttataacactcacaagaatcctgaaaaggatgccaaacacagctgtttgcagacctcc

SEQ ID NO: 769 - RNA, 89 nt, synthetic construct
  source 1..89: mol_type = other RNA
gacagcttatttggaagctgaaatgtgaggtttataacactcacaagaatcctgaaaaggatgccaaactttgcagacctcctgccacc

SEQ ID NO: 770 - RNA, 89 nt, synthetic construct
  source 1..89: mol_type = other RNA
gacagcttatttggaagctgaaatgtgaggtttataacactcacaagaatcctgaaaaggatgccaaacgcaggaggtctgcaaacagc

SEQ ID NO: 771 - RNA, 89 nt, synthetic construct
  source 1..89: mol_type = other RNA
gacagcttatttggaagctgaaatgtgaggtttataacactcacaagaatcctgaaaaggatgccaaaccagacctcctgccaccgcag

SEQ ID NO: 772 - RNA, 89 nt, synthetic construct
  source 1..89: mol_type = other RNA
gacagcttatttggaagctgaaatgtgaggtttataacactcacaagaatcctgaaaaggatgccaaaccggtggcaggaggtctgcaa

SEQ ID NO: 773 - RNA, 89 nt, synthetic construct
  source 1..89: mol_type = other RNA
gacagcttatttggaagctgaaatgtgaggtttataacactcacaagaatcctgaaaaggatgccaaacaatctgcggtggcaggaggt

SEQ ID NO: 774 - RNA, 89 nt, synthetic construct
  source 1..89: mol_type = other RNA
gacagcttatttggaagctgaaatgtgaggtttataacactcacaagaatcctgaaaaggatgccaaacccaccgcagattcaggcttc

SEQ ID NO: 775 - RNA, 89 nt, synthetic construct
  source 1..89: mol_type = other RNA
gacagcttatttggaagctgaaatgtgaggtttataacactcacaagaatcctgaaaaggatgccaaacggaagcctgaatctgcggtg

SEQ ID NO: 776 - RNA, 89 nt, synthetic construct
  source 1..89: mol_type = other RNA
gacagcttatttggaagctgaaatgtgaggtttataacactcacaagaatcctgaaaaggatgccaaaccaggaaaaattgggaagcct

SEQ ID NO: 777 - RNA, 89 nt, synthetic construct
  source 1..89: mol_type = other RNA
gacagcttatttggaagctgaaatgtgaggtttataacactcacaagaatcctgaaaaggatgccaaacttctacaggaaaaattggga

SEQ ID NO: 778 - RNA, 89 nt, synthetic construct
  source 1..89: mol_type = other RNA
gacagcttatttggaagctgaaatgtgaggtttataacactcacaagaatcctgaaaaggatgccaaacccagtattctacaggaaaaa

SEQ ID NO: 779 - RNA, 89 nt, synthetic construct
  source 1..89: mol_type = other RNA
gacagcttatttggaagctgaaatgtgaggtttataacactcacaagaatcctgaaaaggatgccaaactagaatactggcatctgttt

SEQ ID NO: 780 - RNA, 89 nt, synthetic construct
  source 1..89: mol_type = other RNA
gacagcttatttggaagctgaaatgtgaggtttataacactcacaagaatcctgaaaaggatgccaaacgaatactggcatctgttttt

SEQ ID NO: 781 - RNA, 89 nt, synthetic construct
  source 1..89: mol_type = other RNA
gacagcttatttggaagctgaaatgtgaggtttataacactcacaagaatcctgaaaaggatgccaaacctggcatctgtttttgagga

SEQ ID NO: 782 - RNA, 89 nt, synthetic construct
  source 1..89: mol_type = other RNA
gacagcttatttggaagctgaaatgtgaggtttataacactcacaagaatcctgaaaaggatgccaaacgcatctgtttttgaggattg

SEQ ID NO: 783 - RNA, 89 nt, synthetic construct
  source 1..89: mol_type = other RNA
gacagcttatttggaagctgaaatgtgaggtttataacactcacaagaatcctgaaaaggatgccaaactttttgaggattgctgaatt

SEQ ID NO: 784 - RNA, 89 nt, synthetic construct
  source 1..89: mol_type = other RNA
gacagcttatttggaagctgaaatgtgaggtttataacactcacaagaatcctgaaaaggatgccaaacattcagcaatcctcaaaaac

SEQ ID NO: 785 - RNA, 89 nt, synthetic construct
  source 1..89: mol_type = other RNA
gacagcttatttggaagctgaaatgtgaggtttataacactcacaagaatcctgaaaaggatgccaaacaggattgctgaattatttct

SEQ ID NO: 786 - RNA, 89 nt, synthetic construct
  source 1..89: mol_type = other RNA
gacagcttatttggaagctgaaatgtgaggtttataacactcacaagaatcctgaaaaggatgccaaacctgaattatttcttccccag

SEQ ID NO: 787 - RNA, 89 nt, synthetic construct
  source 1..89: mol_type = other RNA
gacagcttatttggaagctgaaatgtgaggtttataacactcacaagaatcctgaaaaggatgccaaacgggaagaaataattcagcaa

SEQ ID NO: 788 - RNA, 89 nt, synthetic construct
  source 1..89: mol_type = other RNA
gacagcttatttggaagctgaaatgtgaggtttataacactcacaagaatcctgaaaaggatgccaaacaattatttcttccccagttg

SEQ ID NO: 789 - RNA, 89 nt, synthetic construct
  source 1..89: mol_type = other RNA
gacagcttatttggaagctgaaatgtgaggtttataacactcacaagaatcctgaaaaggatgccaaaccaactggggaagaaataatt

SEQ ID NO: 790 - RNA, 89 nt, synthetic construct
  source 1..89: mol_type = other RNA
gacagcttatttggaagctgaaatgtgaggtttataacactcacaagaatcctgaaaaggatgccaaactttcttccccagttgcattc

SEQ ID NO: 791 - RNA, 89 nt, synthetic construct
  source 1..89: mol_type = other RNA
gacagcttatttggaagctgaaatgtgaggtttataacactcacaagaatcctgaaaaggatgccaaacaatgcaactggggaagaaat

SEQ ID NO: 792 - RNA, 89 nt, synthetic construct
  source 1..89: mol_type = other RNA
gacagcttatttggaagctgaaatgtgaggtttataacactcacaagaatcctgaaaaggatgccaaactcagaacattgaatgcaact

SEQ ID NO: 793 - RNA, 89 nt, synthetic construct
  source 1..89: mol_type = other RNA
gacagcttatttggaagctgaaatgtgaggtttataacactcacaagaatcctgaaaaggatgccaaaccattcaatgttctgacaaca

SEQ ID NO: 794 - RNA, 89 nt, synthetic construct
  source 1..89: mol_type = other RNA
gacagcttatttggaagctgaaatgtgaggtttataacactcacaagaatcctgaaaaggatgccaaacttgtcagaacattgaatgca

SEQ ID NO: 795 - RNA, 89 nt, synthetic construct
  source 1..89: mol_type = other RNA
gacagcttatttggaagctgaaatgtgaggtttataacactcacaagaatcctgaaaaggatgccaaacttctgacaacagtttgccgc

SEQ ID NO: 796 - RNA, 89 nt, synthetic construct
  source 1..89: mol_type = other RNA
gacagcttatttggaagctgaaatgtgaggtttataacactcacaagaatcctgaaaaggatgccaaacacaacagtttgccgctgccc

SEQ ID NO: 797 - RNA, 89 nt, synthetic construct
  source 1..89: mol_type = other RNA
gacagcttatttggaagctgaaatgtgaggtttataacactcacaagaatcctgaaaaggatgccaaacggcagcggcaaactgttgtc

SEQ ID NO: 798 - RNA, 89 nt, synthetic construct
  source 1..89: mol_type = other RNA
gacagcttatttggaagctgaaatgtgaggtttataacactcacaagaatcctgaaaaggatgccaaacgcattgggcagcggcaaact

SEQ ID NO: 799 - RNA, 89 nt, synthetic construct
  source 1..89: mol_type = other RNA
gacagcttatttggaagctgaaatgtgaggtttataacactcacaagaatcctgaaaaggatgccaaacccgctgcccaatgccatcct

SEQ ID NO: 800 - RNA, 89 nt, synthetic construct
  source 1..89: mol_type = other RNA
gacagcttatttggaagctgaaatgtgaggtttataacactcacaagaatcctgaaaaggatgccaaaccccaatgccatcctggagtt

SEQ ID NO: 801 - RNA, 89 nt, synthetic construct
  source 1..89: mol_type = other RNA
gacagcttatttggaagctgaaatgtgaggtttataacactcacaagaatcctgaaaaggatgccaaacagataccatttgtatttagc

SEQ ID NO: 802 - RNA, 89 nt, synthetic construct
  source 1..89: mol_type = other RNA
gacagcttatttggaagctgaaatgtgaggtttataacactcacaagaatcctgaaaaggatgccaaacaatacaaatggtatcttaag

SEQ ID NO: 803 - RNA, 89 nt, synthetic construct
  source 1..89: mol_type = other RNA
gacagcttatttggaagctgaaatgtgaggtttataacactcacaagaatcctgaaaaggatgccaaacctaaatacaaatggtatctt

SEQ ID NO: 804 - RNA, 89 nt, synthetic construct
  source 1..89: mol_type = other RNA
gacagcttatttggaagctgaaatgtgaggtttataacactcacaagaatcctgaaaaggatgccaaacccatttgtatttagcatgtt

SEQ ID NO: 805 - RNA, 89 nt, synthetic construct
  source 1..89: mol_type = other RNA
gacagcttatttggaagctgaaatgtgaggtttataacactcacaagaatcctgaaaaggatgccaaacggaacatgctaaatacaaat

SEQ ID NO: 806 - RNA, 89 nt, synthetic construct
  source 1..89: mol_type = other RNA
gacagcttatttggaagctgaaatgtgaggtttataacactcacaagaatcctgaaaaggatgccaaactatttagcatgttcccaatt

SEQ ID NO: 807 - RNA, 89 nt, synthetic construct
  source 1..89: mol_type = other RNA
gacagcttatttggaagctgaaatgtgaggtttataacactcacaagaatcctgaaaaggatgccaaactttagcatgttcccaattct

SEQ ID NO: 808 - RNA, 89 nt, synthetic construct
  source 1..89: mol_type = other RNA
gacagcttatttggaagctgaaatgtgaggtttataacactcacaagaatcctgaaaaggatgccaaacgcatgttcccaattctcagg

SEQ ID NO: 809 - RNA, 89 nt, synthetic construct
  source 1..89: mol_type = other RNA
gacagcttatttggaagctgaaatgtgaggtttataacactcacaagaatcctgaaaaggatgccaaacagaattgggaacatgctaaa

SEQ ID NO: 810 - RNA, 89 nt, synthetic construct
  source 1..89: mol_type = other RNA
gacagcttatttggaagctgaaatgtgaggtttataacactcacaagaatcctgaaaaggatgccaaacttcccaattctcaggaattt

SEQ ID NO: 811 - RNA, 89 nt, synthetic construct
  source 1..89: mol_type = other RNA
gacagcttatttggaagctgaaatgtgaggtttataacactcacaagaatcctgaaaaggatgccaaactgtctttctgagaaactgtt

SEQ ID NO: 812 - RNA, 89 nt, synthetic construct
  source 1..89: mol_type = other RNA
gacagcttatttggaagctgaaatgtgaggtttataacactcacaagaatcctgaaaaggatgccaaactctttctgagaaactgttca

SEQ ID NO: 813 - RNA, 89 nt, synthetic construct
  source 1..89: mol_type = other RNA
gacagcttatttggaagctgaaatgtgaggtttataacactcacaagaatcctgaaaaggatgccaaacaacagtttctcagaaagaca

SEQ ID NO: 814 - RNA, 89 nt, synthetic construct
  source 1..89: mol_type = other RNA
gacagcttatttggaagctgaaatgtgaggtttataacactcacaagaatcctgaaaaggatgccaaacagaaactgttcagcttctgt

SEQ ID NO: 815 - RNA, 89 nt, synthetic construct
  source 1..89: mol_type = other RNA
gacagcttatttggaagctgaaatgtgaggtttataacactcacaagaatcctgaaaaggatgccaaacacagaagctgaacagtttct

SEQ ID NO: 816 - RNA, 89 nt, synthetic construct
  source 1..89: mol_type = other RNA
gacagcttatttggaagctgaaatgtgaggtttataacactcacaagaatcctgaaaaggatgccaaacttcagcttctgttagccact

SEQ ID NO: 817 - RNA, 89 nt, synthetic construct
  source 1..89: mol_type = other RNA
gacagcttatttggaagctgaaatgtgaggtttataacactcacaagaatcctgaaaaggatgccaaacgctaacagaagctgaacagt

SEQ ID NO: 818 - RNA, 89 nt, synthetic construct
  source 1..89: mol_type = other RNA
gacagcttatttggaagctgaaatgtgaggtttataacactcacaagaatcctgaaaaggatgccaaacatcagtggctaacagaagct

SEQ ID NO: 819 - RNA, 89 nt, synthetic construct
  source 1..89: mol_type = other RNA
gacagcttatttggaagctgaaatgtgaggtttataacactcacaagaatcctgaaaaggatgccaaacttagccactgattaaatatc

SEQ ID NO: 820 - RNA, 89 nt, synthetic construct
  source 1..89: mol_type = other RNA
gacagcttatttggaagctgaaatgtgaggtttataacactcacaagaatcctgaaaaggatgccaaactttaatcagtggctaacaga

SEQ ID NO: 821 - RNA, 89 nt, synthetic construct
  source 1..89: mol_type = other RNA
gacagcttatttggaagctgaaatgtgaggtttataacactcacaagaatcctgaaaaggatgccaaacgccactgattaaatatcttt

SEQ ID NO: 822 - RNA, 89 nt, synthetic construct
  source 1..89: mol_type = other RNA
gacagcttatttggaagctgaaatgtgaggtttataacactcacaagaatcctgaaaaggatgccaaacaagatatttaatcagtggct

SEQ ID NO: 823 - RNA, 89 nt, synthetic construct
  source 1..89: mol_type = other RNA
gacagcttatttggaagctgaaatgtgaggtttataacactcacaagaatcctgaaaaggatgccaaactaaagatatttaatcagtgg

SEQ ID NO: 824 - RNA, 89 nt, synthetic construct
  source 1..89: mol_type = other RNA
gacagcttatttggaagctgaaatgtgaggtttataacactcacaagaatcctgaaaaggatgccaaactatcataatgaaaacgccgc

SEQ ID NO: 825 - RNA, 89 nt, synthetic construct
  source 1..89: mol_type = other RNA
gacagcttatttggaagctgaaatgtgaggtttataacactcacaagaatcctgaaaaggatgccaaactcataatgaaaacgccgcca

SEQ ID NO: 826 - RNA, 89 nt, synthetic construct
  source 1..89: mol_type = other RNA
gacagcttatttggaagctgaaatgtgaggtttataacactcacaagaatcctgaaaaggatgccaaacgcggcgttttcattatgata

SEQ ID NO: 827 - RNA, 89 nt, synthetic construct
  source 1..89: mol_type = other RNA
gacagcttatttggaagctgaaatgtgaggtttataacactcacaagaatcctgaaaaggatgccaaacatgaaaacgccgccatttct

SEQ ID NO: 828 - RNA, 89 nt, synthetic construct
  source 1..89: mol_type = other RNA
gacagcttatttggaagctgaaatgtgaggtttataacactcacaagaatcctgaaaaggatgccaaacaaaacgccgccatttctcaa

SEQ ID NO: 829 - RNA, 89 nt, synthetic construct
  source 1..89: mol_type = other RNA
gacagcttatttggaagctgaaatgtgaggtttataacactcacaagaatcctgaaaaggatgccaaacagaaatggcggcgttttcat

SEQ ID NO: 830 - RNA, 89 nt, synthetic construct
  source 1..89: mol_type = other RNA
gacagcttatttggaagctgaaatgtgaggtttataacactcacaagaatcctgaaaaggatgccaaacttgagaaatggcggcgtttt

SEQ ID NO: 831 - RNA, 89 nt, synthetic construct
  source 1..89: mol_type = other RNA
gacagcttatttggaagctgaaatgtgaggtttataacactcacaagaatcctgaaaaggatgccaaacacagatctgttgagaaatgg

SEQ ID NO: 832 - RNA, 11 nt, synthetic construct
  source 1..11: mol_type = other RNA
ttaaaggattc

SEQ ID NO: 833 - RNA, 11 nt, synthetic construct
  source 1..11: mol_type = other RNA
ttgaatgaaat

SEQ ID NO: 834 - RNA, 11 nt, synthetic construct
  source 1..11: mol_type = other RNA
tgttcttgtac

SEQ ID NO: 835 - RNA, 11 nt, synthetic construct
  source 1..11: mol_type = other RNA
tgaagtacaag

SEQ ID NO: 836 - RNA, 11 nt, synthetic construct
  source 1..11: mol_type = other RNA
ttgtacttcat

SEQ ID NO: 837 - RNA, 11 nt, synthetic construct
  source 1..11: mol_type = other RNA
gtgggatgaag

SEQ ID NO: 838 - RNA, 11 nt, synthetic construct
  source 1..11: mol_type = other RNA
cagtgggatga

SEQ ID NO: 839 - RNA, 11 nt, synthetic construct
  source 1..11: mol_type = other RNA
gcaatcaagag

SEQ ID NO: 840 - RNA, 11 nt, synthetic construct
  source 1..11: mol_type = other RNA
tggtcttgttt

SEQ ID NO: 841 - RNA, 11 nt, synthetic construct
  source 1..11: mol_type = other RNA
cattaccgctg

SEQ ID NO: 842 - RNA, 11 nt, synthetic construct
  source 1..11: mol_type = other RNA
taatgagttct

SEQ ID NO: 843 - RNA, 11 nt, synthetic construct
  source 1..11: mol_type = other RNA
tggaagaactc

SEQ ID NO: 844 - RNA, 11 nt, synthetic construct
  source 1..11: mol_type = other RNA
agttggaagaa

SEQ ID NO: 845 - RNA, 11 nt, synthetic construct
  source 1..11: mol_type = other RNA
gacgcctctgt

SEQ ID NO: 846 - RNA, 11 nt, synthetic construct
  source 1..11: mol_type = other RNA
acagaggcgtc

SEQ ID NO: 847 - RNA, 11 nt, synthetic construct
  source 1..11: mol_type = other RNA
gttccaaatcc

SEQ ID NO: 848 - RNA, 11 nt, synthetic construct
  source 1..11: mol_type = other RNA
gcaggatttgg

SEQ ID NO: 849 - RNA, 11 nt, synthetic construct
  source 1..11: mol_type = other RNA
tactctggtga

SEQ ID NO: 850 - RNA, 11 nt, synthetic construct
  source 1..11: mol_type = other RNA
gttactctggt

SEQ ID NO: 851 - RNA, 11 nt, synthetic construct
  source 1..11: mol_type = other RNA
gtaacagtctg

SEQ ID NO: 852 - RNA, 11 nt, synthetic construct
  source 1..11: mol_type = other RNA
attcagcaatc

SEQ ID NO: 853 - RNA, 11 nt, synthetic construct
  source 1..11: mol_type = other RNA
agaacattgaa

SEQ ID NO: 854 - RNA, 11 nt, synthetic construct
  source 1..11: mol_type = other RNA
cgctgcccaat

SEQ ID NO: 855 - RNA, 11 nt, synthetic construct
  source 1..11: mol_type = other RNA
ggatggcattg

SEQ ID NO: 856 - RNA, 16 nt, synthetic construct
  source 1..16: mol_type = other RNA
ttgaatgaagtacatg

SEQ ID NO: 857 - RNA, 16 nt, synthetic construct
  source 1..16: mol_type = other RNA
cggaggcaagtaccag

SEQ ID NO: 858 - RNA, 16 nt, synthetic construct
  source 1..16: mol_type = other RNA
cggttctgagtacagg

SEQ ID NO: 859 - RNA, 16 nt, synthetic construct
  source 1..16: mol_type = other RNA
gaatcagtggtacgga

SEQ ID NO: 860 - RNA, 16 nt, synthetic construct
  source 1..16: mol_type = other RNA
ctgaaggtggtacttc

SEQ ID NO: 861 - RNA, 16 nt, synthetic construct
  source 1..16: mol_type = other RNA
aattcagaagtactca

SEQ ID NO: 862 - RNA, 16 nt, synthetic construct
  source 1..16: mol_type = other RNA
agaattcaggtacaat

SEQ ID NO: 863 - RNA, 17 nt, synthetic construct
  source 1..17: mol_type = other RNA
aaaacaagaggtaccca

SEQ ID NO: 864 - RNA, 17 nt, synthetic construct
  source 1..17: mol_type = other RNA
cctcttgatggtactgc

SEQ ID NO: 865 - RNA, 17 nt, synthetic construct
  source 1..17: mol_type = other RNA
gttggaagaggtacact

SEQ ID NO: 866 - RNA, 17 nt, synthetic construct
  source 1..17: mol_type = other RNA
tttgggcagggtaccgg

SEQ ID NO: 867 - RNA, 17 nt, synthetic construct
  source 1..17: mol_type = other RNA
ggcgtccccggtacagt

SEQ ID NO: 868 - RNA, 17 nt, synthetic construct
  source 1..17: mol_type = other RNA
agaggcgtcggtacccc

SEQ ID NO: 869 - RNA, 17 nt, synthetic construct
  source 1..17: mol_type = other RNA
cttccaactggtacggg

SEQ ID NO: 870 - RNA, 17 nt, synthetic construct
  source 1..17: mol_type = other RNA
gcaggatttggtacgga

SEQ ID NO: 871 - RNA, 17 nt, synthetic construct
  source 1..17: mol_type = other RNA
ggggacgccggtactct

SEQ ID NO: 872 - RNA, 17 nt, synthetic construct
  source 1..17: mol_type = other RNA
acaggcaacggtacaat

SEQ ID NO: 873 - RNA, 16 nt, synthetic construct
  source 1..16: mol_type = other RNA
tactcagacgtactgt

SEQ ID NO: 874 - RNA, 16 nt, synthetic construct
  source 1..16: mol_type = other RNA
cctactcaggtacact

SEQ ID NO: 875 - RNA, 16 nt, synthetic construct
  source 1..16: mol_type = other RNA
tgtgtcaccgtacaga

SEQ ID NO: 876 - RNA, 16 nt, synthetic construct
  source 1..16: mol_type = other RNA
ggggaagaagtacata

SEQ ID NO: 877 - RNA, 16 nt, synthetic construct
  source 1..16: mol_type = other RNA
caaactgttgtacgtc

SEQ ID NO: 878 - RNA, 16 nt, synthetic construct
  source 1..16: mol_type = other RNA
acaacagttgtactgc

SEQ ID NO: 879 - RNA, 16 nt, synthetic construct
  source 1..16: mol_type = other RNA
acaggaactgtaccca

SEQ ID NO: 880 - RNA, 27 nt, synthetic construct
  source 1..27: mol_type = other RNA
ttgaatgaagtacatgttaaaggattc

SEQ ID NO: 881 - RNA, 27 nt, synthetic construct
  source 1..27: mol_type = other RNA
cggaggcaagtaccagttgaatgaaat

SEQ ID NO: 882 - RNA, 27 nt, synthetic construct
  source 1..27: mol_type = other RNA
cggttctgagtacaggtgttcttgtac

SEQ ID NO: 883 - RNA, 27 nt, synthetic construct
  source 1..27: mol_type = other RNA
gaatcagtggtacggatgaagtacaag

SEQ ID NO: 884 - RNA, 27 nt, synthetic construct
  source 1..27: mol_type = other RNA
ctgaaggtggtacttcttgtacttcat

SEQ ID NO: 885 - RNA, 27 nt, synthetic construct
  source 1..27: mol_type = other RNA
aattcagaagtactcagtgggatgaag

SEQ ID NO: 886 - RNA, 27 nt, synthetic construct
  source 1..27: mol_type = other RNA
agaattcaggtacaatcagtgggatga

SEQ ID NO: 887 - RNA, 28 nt, synthetic construct
  source 1..28: mol_type = other RNA
aaaacaagaggtacccagcaatcaagag

SEQ ID NO: 888 - RNA, 28 nt, synthetic construct
  source 1..28: mol_type = other RNA
cctcttgatggtactgctggtcttgttt

SEQ ID NO: 889 - RNA, 28 nt, synthetic construct
  source 1..28: mol_type = other RNA
gttggaagaggtacactcattaccgctg

SEQ ID NO: 890 - RNA, 28 nt, synthetic construct
  source 1..28: mol_type = other RNA
tttgggcagggtaccggtaatgagttct

SEQ ID NO: 891 - RNA, 28 nt, synthetic construct
  source 1..28: mol_type = other RNA
ggcgtccccggtacagttggaagaactc

SEQ ID NO: 892 - RNA, 28 nt, synthetic construct
  source 1..28: mol_type = other RNA
agaggcgtcggtaccccagttggaagaa

SEQ ID NO: 893 - RNA, 28 nt, synthetic construct
  source 1..28: mol_type = other RNA
cttccaactggtacggggacgcctctgt

SEQ ID NO: 894 - RNA, 28 nt, synthetic construct
  source 1..28: mol_type = other RNA
gcaggatttggtacggaacagaggcgtc

SEQ ID NO: 895 - RNA, 28 nt, synthetic construct
  source 1..28: mol_type = other RNA
ggggacgccggtactctgttccaaatcc

SEQ ID NO: 896 - RNA, 28 nt, synthetic construct
  source 1..28: mol_type = other RNA
acaggcaacggtacaatgcaggatttgg

SEQ ID NO: 897 - RNA, 27 nt, synthetic construct
  source 1..27: mol_type = other RNA
tactcagacgtactgttactctggtga

SEQ ID NO: 898 - RNA, 27 nt, synthetic construct
  source 1..27: mol_type = other RNA
cctactcaggtacactgttactctggt

SEQ ID NO: 899 - RNA, 27 nt, synthetic construct
  source 1..27: mol_type = other RNA
tgtgtcaccgtacagagtaacagtctg

SEQ ID NO: 900 - RNA, 27 nt, synthetic construct
  source 1..27: mol_type = other RNA
ggggaagaagtacataattcagcaatc

SEQ ID NO: 901 - RNA, 27 nt, synthetic construct
  source 1..27: mol_type = other RNA
caaactgttgtacgtcagaacattgaa

SEQ ID NO: 902 - RNA, 27 nt, synthetic construct
  source 1..27: mol_type = other RNA
acaacagttgtactgccgctgcccaat

SEQ ID NO: 903 - RNA, 27 nt, synthetic construct
  source 1..27: mol_type = other RNA
acaggaactgtacccaggatggcattg

SEQ ID NO: 904 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttgaatgaagtacatgttaaaggattcaaattaacagtggccgcggtcggcgtggactgtagaacactgccaatgccggtcccaagcccggataaaagtggagggtacagtccacgctctagagcggacttcggtccgc

SEQ ID NO: 905 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccggaggcaagtaccagttgaatgaaataaattaacagtggccgcggtcggcgtggactgtagaacactgccaatgccggtcccaagcccggataaaagtggagggtacagtccacgctctagagcggacttcggtccgc

SEQ ID NO: 906 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccggttctgagtacaggtgttcttgtacaaattaacagtggccgcggtcggcgtggactgtagaacactgccaatgccggtcccaagcccggataaaagtggagggtacagtccacgctctagagcggacttcggtccgc

SEQ ID NO: 907 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgaatcagtggtacggatgaagtacaagaaattaacagtggccgcggtcggcgtggactgtagaacactgccaatgccggtcccaagcccggataaaagtggagggtacagtccacgctctagagcggacttcggtccgc

SEQ ID NO: 908 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcctgaaggtggtacttcttgtacttcataaattaacagtggccgcggtcggcgtggactgtagaacactgccaatgccggtcccaagcccggataaaagtggagggtacagtccacgctctagagcggacttcggtccgc

SEQ ID NO: 909 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcaattcagaagtactcagtgggatgaagaaattaacagtggccgcggtcggcgtggactgtagaacactgccaatgccggtcccaagcccggataaaagtggagggtacagtccacgctctagagcggacttcggtccgc

SEQ ID NO: 910 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcagaattcaggtacaatcagtgggatgaaaattaacagtggccgcggtcggcgtggactgtagaacactgccaatgccggtcccaagcccggataaaagtggagggtacagtccacgctctagagcggacttcggtccgc

SEQ ID NO: 911 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcaaaacaagaggtacccagcaatcaagagaaattaacagtggccgcggtcggcgtggactgtagaacactgccaatgccggtcccaagcccggataaaagtggagggtacagtccacgctctagagcggacttcggtccgc

SEQ ID NO: 912 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccctcttgatggtactgctggtcttgtttaaattaacagtggccgcggtcggcgtggactgtagaacactgccaatgccggtcccaagcccggataaaagtggagggtacagtccacgctctagagcggacttcggtccgc

SEQ ID NO: 913 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgttggaagaggtacactcattaccgctgaaattaacagtggccgcggtcggcgtggactgtagaacactgccaatgccggtcccaagcccggataaaagtggagggtacagtccacgctctagagcggacttcggtccgc

SEQ ID NO: 914 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctttgggcagggtaccggtaatgagttctaaattaacagtggccgcggtcggcgtggactgtagaacactgccaatgccggtcccaagcccggataaaagtggagggtacagtccacgctctagagcggacttcggtccgc

SEQ ID NO: 915 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcggcgtccccggtacagttggaagaactcaaattaacagtggccgcggtcggcgtggactgtagaacactgccaatgccggtcccaagcccggataaaagtggagggtacagtccacgctctagagcggacttcggtccgc

SEQ ID NO: 916 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcagaggcgtcggtaccccagttggaagaaaaattaacagtggccgcggtcggcgtggactgtagaacactgccaatgccggtcccaagcccggataaaagtggagggtacagtccacgctctagagcggacttcggtccgc

SEQ ID NO: 917 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccttccaactggtacggggacgcctctgtaaattaacagtggccgcggtcggcgtggactgtagaacactgccaatgccggtcccaagcccggataaaagtggagggtacagtccacgctctagagcggacttcggtccgc

SEQ ID NO: 918 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgcaggatttggtacggaacagaggcgtcaaattaacagtggccgcggtcggcgtggactgtagaacactgccaatgccggtcccaagcccggataaaagtggagggtacagtccacgctctagagcggacttcggtccgc

SEQ ID NO: 919 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcggggacgccggtactctgttccaaatccaaattaacagtggccgcggtcggcgtggactgtagaacactgccaatgccggtcccaagcccggataaaagtggagggtacagtccacgctctagagcggacttcggtccgc

SEQ ID NO: 920 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcacaggcaacggtacaatgcaggatttggaaattaacagtggccgcggtcggcgtggactgtagaacactgccaatgccggtcccaagcccggataaaagtggagggtacagtccacgctctagagcggacttcggtccgc

SEQ ID NO: 921 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctactcagacgtactgttactctggtgaaaattaacagtggccgcggtcggcgtggactgtagaacactgccaatgccggtcccaagcccggataaaagtggagggtacagtccacgctctagagcggacttcggtccgc

SEQ ID NO: 922 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccctactcaggtacactgttactctggtaaattaacagtggccgcggtcggcgtggactgtagaacactgccaatgccggtcccaagcccggataaaagtggagggtacagtccacgctctagagcggacttcggtccgc

SEQ ID NO: 923 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctgtgtcaccgtacagagtaacagtctgaaattaacagtggccgcggtcggcgtggactgtagaacactgccaatgccggtcccaagcccggataaaagtggagggtacagtccacgctctagagcggacttcggtccgc

SEQ ID NO: 924 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcggggaagaagtacataattcagcaatcaaattaacagtggccgcggtcggcgtggactgtagaacactgccaatgccggtcccaagcccggataaaagtggagggtacagtccacgctctagagcggacttcggtccgc

SEQ ID NO: 925 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccaaactgttgtacgtcagaacattgaaaaattaacagtggccgcggtcggcgtggactgtagaacactgccaatgccggtcccaagcccggataaaagtggagggtacagtccacgctctagagcggacttcggtccgc

SEQ ID NO: 926 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcacaacagttgtactgccgctgcccaataaattaacagtggccgcggtcggcgtggactgtagaacactgccaatgccggtcccaagcccggataaaagtggagggtacagtccacgctctagagcggacttcggtccgc

SEQ ID NO: 927 - RNA, 279 nt, synthetic construct
  source 1..279: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcacaggaactgtacccaggatggcattgaaattaacagtggccgcggtcggcgtggactgtagaacactgccaatgccggtcccaagcccggataaaagtggagggtacagtccacgctctagagcggacttcggtccgc

SEQ ID NO: 928 - RNA, 17 nt, synthetic construct
  source 1..17: mol_type = other RNA
gtttctgtgattttctt

SEQ ID NO: 929 - RNA, 17 nt, synthetic construct
  source 1..17: mol_type = other RNA
ctgtgattttcttttgg

SEQ ID NO: 930 - RNA, 17 nt, synthetic construct
  source 1..17: mol_type = other RNA
tgtgattttcttttgga

SEQ ID NO: 931 - RNA, 17 nt, synthetic construct
  source 1..17: mol_type = other RNA
tcttttggattgcatct

SEQ ID NO: 932 - RNA, 17 nt, synthetic construct
  source 1..17: mol_type = other RNA
cttttggattgcatcta

SEQ ID NO: 933 - RNA, 17 nt, synthetic construct
  source 1..17: mol_type = other RNA
ttttggattgcatctac

SEQ ID NO: 934 - RNA, 17 nt, synthetic construct
  source 1..17: mol_type = other RNA
tggattgcatctactgt

SEQ ID NO: 935 - RNA, 17 nt, synthetic construct
  source 1..17: mol_type = other RNA
ggattgcatctactgta

SEQ ID NO: 936 - RNA, 17 nt, synthetic construct
  source 1..17: mol_type = other RNA
gattgcatctactgtat

SEQ ID NO: 937 - RNA, 17 nt, synthetic construct
  source 1..17: mol_type = other RNA
catctactgtataggga

SEQ ID NO: 938 - RNA, 17 nt, synthetic construct
  source 1..17: mol_type = other RNA
agtcatggaaggagggt

SEQ ID NO: 939 - RNA, 17 nt, synthetic construct
  source 1..17: mol_type = other RNA
catgactcaagcttggc

SEQ ID NO: 940 - RNA, 17 nt, synthetic construct
  source 1..17: mol_type = other RNA
gctctggcctgtcctaa

SEQ ID NO: 941 - RNA, 17 nt, synthetic construct
  source 1..17: mol_type = other RNA
ggacaggccagagccaa

SEQ ID NO: 942 - RNA, 17 nt, synthetic construct
  source 1..17: mol_type = other RNA
ttccttagcttccagcc

SEQ ID NO: 943 - RNA, 17 nt, synthetic construct
  source 1..17: mol_type = other RNA
cttagcttccagccatt

SEQ ID NO: 944 - RNA, 17 nt, synthetic construct
  source 1..17: mol_type = other RNA
gcttccagccattgtgt

SEQ ID NO: 945 - RNA, 17 nt, synthetic construct
  source 1..17: mol_type = other RNA
cagccattgtgttgaat

SEQ ID NO: 946 - RNA, 17 nt, synthetic construct
  source 1..17: mol_type = other RNA
aacacaatggctggaag

SEQ ID NO: 947 - RNA, 17 nt, synthetic construct
  source 1..17: mol_type = other RNA
aaggattcaacacaatg

SEQ ID NO: 948 - RNA, 17 nt, synthetic construct
  source 1..17: mol_type = other RNA
tgttgaatcctttaaca

SEQ ID NO: 949 - RNA, 17 nt, synthetic construct
  source 1..17: mol_type = other RNA
aacatttcattcaactg

SEQ ID NO: 950 - RNA, 17 nt, synthetic construct
  source 1..17: mol_type = other RNA
acatttcattcaactgt

SEQ ID NO: 951 - RNA, 17 nt, synthetic construct
  source 1..17: mol_type = other RNA
cattcaactgttgcctc

SEQ ID NO: 952 - RNA, 17 nt, synthetic construct
  source 1..17: mol_type = other RNA
attcaactgttgcctcc

SEQ ID NO: 953 - RNA, 17 nt, synthetic construct
  source 1..17: mol_type = other RNA
aactgttgcctccggtt

SEQ ID NO: 954 - RNA, 17 nt, synthetic construct
  source 1..17: mol_type = other RNA
agaaccggaggcaacag

SEQ ID NO: 955 - RNA, 17 nt, synthetic construct
  source 1..17: mol_type = other RNA
cctccggttctgaaggt

SEQ ID NO: 956 - RNA, 17 nt, synthetic construct
  source 1..17: mol_type = other RNA
tgaaggtgttcttgtac

SEQ ID NO: 957 - RNA, 17 nt, synthetic construct
  source 1..17: mol_type = other RNA
ttgtacttcatcccact

SEQ ID NO: 958 - RNA, 17 nt, synthetic construct
  source 1..17: mol_type = other RNA
tacttcatcccactgat

SEQ ID NO: 959 - RNA, 17 nt, synthetic construct
  source 1..17: mol_type = other RNA
atcccactgattctgaa

SEQ ID NO: 960 - RNA, 17 nt, synthetic construct
  source 1..17: mol_type = other RNA
agaatcagtgggatgaa

SEQ ID NO: 961 - RNA, 17 nt, synthetic construct
  source 1..17: mol_type = other RNA
ttctagcctcttgattg

SEQ ID NO: 962 - RNA, 17 nt, synthetic construct
  source 1..17: mol_type = other RNA
tagcctcttgattgctg

SEQ ID NO: 963 - RNA, 17 nt, synthetic construct
  source 1..17: mol_type = other RNA
attgctggtcttgtttt

SEQ ID NO: 964 - RNA, 17 nt, synthetic construct
  source 1..17: mol_type = other RNA
ctggtcttgtttttcaa

SEQ ID NO: 965 - RNA, 17 nt, synthetic construct
  source 1..17: mol_type = other RNA
aaaaacaagaccagcaa

SEQ ID NO: 966 - RNA, 17 nt, synthetic construct
  source 1..17: mol_type = other RNA
gaaaaacaagaccagca

SEQ ID NO: 967 - RNA, 17 nt, synthetic construct
  source 1..17: mol_type = other RNA
tttttcaaattttgggc

SEQ ID NO: 968 - RNA, 17 nt, synthetic construct
  source 1..17: mol_type = other RNA
ttcaaattttgggcagc

SEQ ID NO: 969 - RNA, 17 nt, synthetic construct
  source 1..17: mol_type = other RNA
tcaaattttgggcagcg

SEQ ID NO: 970 - RNA, 17 nt, synthetic construct
  source 1..17: mol_type = other RNA
caaattttgggcagcgg

SEQ ID NO: 971 - RNA, 17 nt, synthetic construct
  source 1..17: mol_type = other RNA
aaattttgggcagcggt

SEQ ID NO: 972 - RNA, 17 nt, synthetic construct
  source 1..17: mol_type = other RNA
ccgctgcccaaaatttg

SEQ ID NO: 973 - RNA, 17 nt, synthetic construct
  source 1..17: mol_type = other RNA
tgggcagcggtaatgag

SEQ ID NO: 974 - RNA, 17 nt, synthetic construct
  source 1..17: mol_type = other RNA
gggcagcggtaatgagt

SEQ ID NO: 975 - RNA, 17 nt, synthetic construct
  source 1..17: mol_type = other RNA
ggcagcggtaatgagtt

SEQ ID NO: 976 - RNA, 17 nt, synthetic construct
  source 1..17: mol_type = other RNA
gaagaactcattaccgc

SEQ ID NO: 977 - RNA, 17 nt, synthetic construct
  source 1..17: mol_type = other RNA
ttccaactggggacgcc

SEQ ID NO: 978 - RNA, 17 nt, synthetic construct
  source 1..17: mol_type = other RNA
caactggggacgcctct

SEQ ID NO: 979 - RNA, 17 nt, synthetic construct
  source 1..17: mol_type = other RNA
gaacagaggcgtcccca

SEQ ID NO: 980 - RNA, 17 nt, synthetic construct
  source 1..17: mol_type = other RNA
ggaacagaggcgtcccc

SEQ ID NO: 981 - RNA, 17 nt, synthetic construct
  source 1..17: mol_type = other RNA
caaatcctgcattgttg

SEQ ID NO: 982 - RNA, 17 nt, synthetic construct
  source 1..17: mol_type = other RNA
tgcttgatgatcatctc

SEQ ID NO: 983 - RNA, 17 nt, synthetic construct
  source 1..17: mol_type = other RNA
atgatcatctcgttgat

SEQ ID NO: 984 - RNA, 17 nt, synthetic construct
  source 1..17: mol_type = other RNA
aggatatcaacgagatg

SEQ ID NO: 985 - RNA, 17 nt, synthetic construct
  source 1..17: mol_type = other RNA
atatcctcaaggtcacc

SEQ ID NO: 986 - RNA, 17 nt, synthetic construct
  source 1..17: mol_type = other RNA
taaaatcacagagggtg

SEQ ID NO: 987 - RNA, 17 nt, synthetic construct
  source 1..17: mol_type = other RNA
tataacttgatcaagca

SEQ ID NO: 988 - RNA, 17 nt, synthetic construct
  source 1..17: mol_type = other RNA
ataacttgatcaagcag

SEQ ID NO: 989 - RNA, 17 nt, synthetic construct
  source 1..17: mol_type = other RNA
taacttgatcaagcaga

SEQ ID NO: 990 - RNA, 17 nt, synthetic construct
  source 1..17: mol_type = other RNA
tctgcttgatcaagtta

SEQ ID NO: 991 - RNA, 17 nt, synthetic construct
  source 1..17: mol_type = other RNA
ctctgcttgatcaagtt

SEQ ID NO: 992 - RNA, 17 nt, synthetic construct
  source 1..17: mol_type = other RNA
atcaagcagagaaagcc

SEQ ID NO: 993 - RNA, 17 nt, synthetic construct
  source 1..17: mol_type = other RNA
ccgactggctttctctg

SEQ ID NO: 994 - RNA, 17 nt, synthetic construct
  source 1..17: mol_type = other RNA
gacagaacttaccgact

SEQ ID NO: 995 - RNA, 17 nt, synthetic construct
  source 1..17: mol_type = other RNA
tgtccaagcccggttga

SEQ ID NO: 996 - RNA, 17 nt, synthetic construct
  source 1..17: mol_type = other RNA
aaccgggcttggacaga

SEQ ID NO: 997 - RNA, 17 nt, synthetic construct
  source 1..17: mol_type = other RNA
caaccgggcttggacag

SEQ ID NO: 998 - RNA, 17 nt, synthetic construct
  source 1..17: mol_type = other RNA
aaatctgccagagcagg

SEQ ID NO: 999 - RNA, 17 nt, synthetic construct
  source 1..17: mol_type = other RNA
gaggtacctgctctggc

SEQ ID NO: 1000 - RNA, 17 nt, synthetic construct
  source 1..17: mol_type = other RNA
atgttggaggtacctgc

SEQ ID NO: 1001 - RNA, 17 nt, synthetic construct
  source 1..17: mol_type = other RNA
cttgatgttggaggtac

SEQ ID NO: 1002 - RNA, 17 nt, synthetic construct
  source 1..17: mol_type = other RNA
ctagtttggagatggca

SEQ ID NO: 1003 - RNA, 17 nt, synthetic construct
  source 1..17: mol_type = other RNA
tagtttggagatggcag

SEQ ID NO: 1004 - RNA, 17 nt, synthetic construct
  source 1..17: mol_type = other RNA
ggagatggcagtttcct

SEQ ID NO: 1005 - RNA, 17 nt, synthetic construct
  source 1..17: mol_type = other RNA
gagatggcagtttcctt

SEQ ID NO: 1006 - RNA, 17 nt, synthetic construct
  source 1..17: mol_type = other RNA
ctaaggaaactgccatc

SEQ ID NO: 1007 - RNA, 17 nt, synthetic construct
  source 1..17: mol_type = other RNA
ccttagtaaccacaggt

SEQ ID NO: 1008 - RNA, 17 nt, synthetic construct
  source 1..17: mol_type = other RNA
cttagtaaccacaggtt

SEQ ID NO: 1009 - RNA, 17 nt, synthetic construct
  source 1..17: mol_type = other RNA
gtaaccacaggttgtgt

SEQ ID NO: 1010 - RNA, 17 nt, synthetic construct
  source 1..17: mol_type = other RNA
ctctggtgacacaacct

SEQ ID NO: 1011 - RNA, 17 nt, synthetic construct
  source 1..17: mol_type = other RNA
tgtcaccagagtaacag

SEQ ID NO: 1012 - RNA, 17 nt, synthetic construct
  source 1..17: mol_type = other RNA
tttctgtctgacagctg

SEQ ID NO: 1013 - RNA, 17 nt, synthetic construct
  source 1..17: mol_type = other RNA
ttctgtctgacagctgt

SEQ ID NO: 1014 - RNA, 17 nt, synthetic construct
  source 1..17: mol_type = other RNA
tctgtctgacagctgtt

SEQ ID NO: 1015 - RNA, 17 nt, synthetic construct
  source 1..17: mol_type = other RNA
ctgtctgacagctgttt

SEQ ID NO: 1016 - RNA, 17 nt, synthetic construct
  source 1..17: mol_type = other RNA
tgtctgacagctgtttg

SEQ ID NO: 1017 - RNA, 17 nt, synthetic construct
  source 1..17: mol_type = other RNA
gcagacctcctgccacc

SEQ ID NO: 1018 - RNA, 17 nt, synthetic construct
  source 1..17: mol_type = other RNA
cagacctcctgccaccg

SEQ ID NO: 1019 - RNA, 17 nt, synthetic construct
  source 1..17: mol_type = other RNA
ggaagcctgaatctgcg

SEQ ID NO: 1020 - RNA, 17 nt, synthetic construct
  source 1..17: mol_type = other RNA
aggcttcccaatttttc

SEQ ID NO: 1021 - RNA, 17 nt, synthetic construct
  source 1..17: mol_type = other RNA
ccaatttttcctgtaga

SEQ ID NO: 1022 - RNA, 17 nt, synthetic construct
  source 1..17: mol_type = other RNA
tacaggaaaaattggga

SEQ ID NO: 1023 - RNA, 17 nt, synthetic construct
  source 1..17: mol_type = other RNA
ttcctgtagaatactgg

SEQ ID NO: 1024 - RNA, 17 nt, synthetic construct
  source 1..17: mol_type = other RNA
tcctgtagaatactggc

SEQ ID NO: 1025 - RNA, 17 nt, synthetic construct
  source 1..17: mol_type = other RNA
cctgtagaatactggca

SEQ ID NO: 1026 - RNA, 17 nt, synthetic construct
  source 1..17: mol_type = other RNA
ctgtagaatactggcat

SEQ ID NO: 1027 - RNA, 17 nt, synthetic construct
  source 1..17: mol_type = other RNA
agcaatcctcaaaaaca

SEQ ID NO: 1028 - RNA, 17 nt, synthetic construct
  source 1..17: mol_type = other RNA
ttgaggattgctgaatt

SEQ ID NO: 1029 - RNA, 17 nt, synthetic construct
  source 1..17: mol_type = other RNA
tgaggattgctgaatta

SEQ ID NO: 1030 - RNA, 17 nt, synthetic construct
  source 1..17: mol_type = other RNA
gaggattgctgaattat

SEQ ID NO: 1031 - RNA, 17 nt, synthetic construct
  source 1..17: mol_type = other RNA
aggattgctgaattatt

SEQ ID NO: 1032 - RNA, 17 nt, synthetic construct
  source 1..17: mol_type = other RNA
ctgaattatttcttccc

SEQ ID NO: 1033 - RNA, 17 nt, synthetic construct
  source 1..17: mol_type = other RNA
tttcttccccagttgca

SEQ ID NO: 1034 - RNA, 17 nt, synthetic construct
  source 1..17: mol_type = other RNA
cttccccagttgcattc

SEQ ID NO: 1035 - RNA, 17 nt, synthetic construct
  source 1..17: mol_type = other RNA
ttccccagttgcattca

SEQ ID NO: 1036 - RNA, 17 nt, synthetic construct
  source 1..17: mol_type = other RNA
aatgcaactggggaaga

SEQ ID NO: 1037 - RNA, 17 nt, synthetic construct
  source 1..17: mol_type = other RNA
cccagttgcattcaatg

SEQ ID NO: 1038 - RNA, 17 nt, synthetic construct
  source 1..17: mol_type = other RNA
cattcaatgttctgaca

SEQ ID NO: 1039 - RNA, 17 nt, synthetic construct
  source 1..17: mol_type = other RNA
tcagaacattgaatgca

SEQ ID NO: 1040 - RNA, 17 nt, synthetic construct
  source 1..17: mol_type = other RNA
aatgttctgacaacagt

SEQ ID NO: 1041 - RNA, 17 nt, synthetic construct
  source 1..17: mol_type = other RNA
tgacaacagtttgccgc

SEQ ID NO: 1042 - RNA, 17 nt, synthetic construct
  source 1..17: mol_type = other RNA
ggcagcggcaaactgtt

SEQ ID NO: 1043 - RNA, 17 nt, synthetic construct
  source 1..17: mol_type = other RNA
gccgctgcccaatgcca

SEQ ID NO: 1044 - RNA, 17 nt, synthetic construct
  source 1..17: mol_type = other RNA
ccgctgcccaatgccat

SEQ ID NO: 1045 - RNA, 17 nt, synthetic construct
  source 1..17: mol_type = other RNA
gtatttagcatgttccc

SEQ ID NO: 1046 - RNA, 17 nt, synthetic construct
  source 1..17: mol_type = other RNA
tatttagcatgttccca

SEQ ID NO: 1047 - RNA, 17 nt, synthetic construct
  source 1..17: mol_type = other RNA
ggaacatgctaaataca

SEQ ID NO: 1048 - RNA, 17 nt, synthetic construct
  source 1..17: mol_type = other RNA
agcatgttcccaattct

SEQ ID NO: 1049 - RNA, 17 nt, synthetic construct
  source 1..17: mol_type = other RNA
gcatgttcccaattctc

SEQ ID NO: 1050 - RNA, 17 nt, synthetic construct
  source 1..17: mol_type = other RNA
ctgagaattgggaacat

SEQ ID NO: 1051 - RNA, 17 nt, synthetic construct
  source 1..17: mol_type = other RNA
ccaattctcaggaattt

SEQ ID NO: 1052 - RNA, 17 nt, synthetic construct
  source 1..17: mol_type = other RNA
tcaggaatttgtgtctt

SEQ ID NO: 1053 - RNA, 17 nt, synthetic construct
  source 1..17: mol_type = other RNA
tcagaaagacacaaatt

SEQ ID NO: 1054 - RNA, 17 nt, synthetic construct
  source 1..17: mol_type = other RNA
gtgtctttctgagaaac

SEQ ID NO: 1055 - RNA, 17 nt, synthetic construct
  source 1..17: mol_type = other RNA
ctcagaaagacacaaat

SEQ ID NO: 1056 - RNA, 17 nt, synthetic construct
  source 1..17: mol_type = other RNA
tgtctttctgagaaact

SEQ ID NO: 1057 - RNA, 17 nt, synthetic construct
  source 1..17: mol_type = other RNA
ctgagaaactgttcagc

SEQ ID NO: 1058 - RNA, 17 nt, synthetic construct
  source 1..17: mol_type = other RNA
tgagaaactgttcagct

SEQ ID NO: 1059 - RNA, 17 nt, synthetic construct
  source 1..17: mol_type = other RNA
agcttctgttagccact

SEQ ID NO: 1060 - RNA, 17 nt, synthetic construct
  source 1..17: mol_type = other RNA
tgttagccactgattaa

SEQ ID NO: 1061 - RNA, 17 nt, synthetic construct
  source 1..17: mol_type = other RNA
atcagtggctaacagaa

SEQ ID NO: 1062 - RNA, 17 nt, synthetic construct
  source 1..17: mol_type = other RNA
aatcagtggctaacaga

SEQ ID NO: 1063 - RNA, 17 nt, synthetic construct
  source 1..17: mol_type = other RNA
gccactgattaaatatc

SEQ ID NO: 1064 - RNA, 17 nt, synthetic construct
  source 1..17: mol_type = other RNA
tatcataatgaaaacgc

SEQ ID NO: 1065 - RNA, 17 nt, synthetic construct
  source 1..17: mol_type = other RNA
agaaatggcggcgtttt

SEQ ID NO: 1066 - RNA, 17 nt, synthetic construct
  source 1..17: mol_type = other RNA
ctcaacagatctgtcaa

SEQ ID NO: 1067 - RNA, 17 nt, synthetic construct
  source 1..17: mol_type = other RNA
tcaacagatctgtcaaa

SEQ ID NO: 1068 - RNA, 17 nt, synthetic construct
  source 1..17: mol_type = other RNA
acagatctgttgagaaa

SEQ ID NO: 1069 - RNA, 17 nt, synthetic construct
  source 1..17: mol_type = other RNA
gacagatctgttgagaa

SEQ ID NO: 1070-1211 - skipped (142 entries)

SEQ ID NO: 1212 - RNA, 37 nt, synthetic construct
  source 1..37: mol_type = other RNA
attgctccttacgaggagacgtttctgtgattttctt

SEQ ID NO: 1213 - RNA, 37 nt, synthetic construct
  source 1..37: mol_type = other RNA
attgctccttacgaggagacctgtgattttcttttgg

SEQ ID NO: 1214 - RNA, 37 nt, synthetic construct
  source 1..37: mol_type = other RNA
attgctccttacgaggagactgtgattttcttttgga

SEQ ID NO: 1215 - RNA, 37 nt, synthetic construct
  source 1..37: mol_type = other RNA
attgctccttacgaggagactcttttggattgcatct

SEQ ID NO: 1216 - RNA, 37 nt, synthetic construct
  source 1..37: mol_type = other RNA
attgctccttacgaggagaccttttggattgcatcta

SEQ ID NO: 1217 - RNA, 37 nt, synthetic construct
  source 1..37: mol_type = other RNA
attgctccttacgaggagacttttggattgcatctac

SEQ ID NO: 1218 - RNA, 37 nt, synthetic construct
  source 1..37: mol_type = other RNA
attgctccttacgaggagactggattgcatctactgt

SEQ ID NO: 1219 - RNA, 37 nt, synthetic construct
  source 1..37: mol_type = other RNA
attgctccttacgaggagacggattgcatctactgta

SEQ ID NO: 1220 - RNA, 37 nt, synthetic construct
  source 1..37: mol_type = other RNA
attgctccttacgaggagacgattgcatctactgtat

SEQ ID NO: 1221 - RNA, 37 nt, synthetic construct
  source 1..37: mol_type = other RNA
attgctccttacgaggagaccatctactgtataggga

SEQ ID NO: 1222 - RNA, 37 nt, synthetic construct
  source 1..37: mol_type = other RNA
attgctccttacgaggagacagtcatggaaggagggt

SEQ ID NO: 1223 - RNA, 37 nt, synthetic construct
  source 1..37: mol_type = other RNA
attgctccttacgaggagaccatgactcaagcttggc

SEQ ID NO: 1224 - RNA, 37 nt, synthetic construct
  source 1..37: mol_type = other RNA
attgctccttacgaggagacgctctggcctgtcctaa

SEQ ID NO: 1225 - RNA, 37 nt, synthetic construct
  source 1..37: mol_type = other RNA
attgctccttacgaggagacggacaggccagagccaa

SEQ ID NO: 1226 - RNA, 37 nt, synthetic construct
  source 1..37: mol_type = other RNA
attgctccttacgaggagacttccttagcttccagcc

SEQ ID NO: 1227 - RNA, 37 nt, synthetic construct
  source 1..37: mol_type = other RNA
attgctccttacgaggagaccttagcttccagccatt

SEQ ID NO: 1228 - RNA, 37 nt, synthetic construct
  source 1..37: mol_type = other RNA
attgctccttacgaggagacgcttccagccattgtgt

SEQ ID NO: 1229 - RNA, 37 nt, synthetic construct
  source 1..37: mol_type = other RNA
attgctccttacgaggagaccagccattgtgttgaat

SEQ ID NO: 1230 - RNA, 37 nt, synthetic construct
  source 1..37: mol_type = other RNA
attgctccttacgaggagacaacacaatggctggaag

SEQ ID NO: 1231 - RNA, 37 nt, synthetic construct
  source 1..37: mol_type = other RNA
attgctccttacgaggagacaaggattcaacacaatg

SEQ ID NO: 1232 - RNA, 37 nt, synthetic construct
  source 1..37: mol_type = other RNA
attgctccttacgaggagactgttgaatcctttaaca

SEQ ID NO: 1233 - RNA, 37 nt, synthetic construct
  source 1..37: mol_type = other RNA
attgctccttacgaggagacaacatttcattcaactg

SEQ ID NO: 1234 - RNA, 37 nt, synthetic construct
  source 1..37: mol_type = other RNA
attgctccttacgaggagacacatttcattcaactgt

SEQ ID NO: 1235 - RNA, 37 nt, synthetic construct
  source 1..37: mol_type = other RNA
attgctccttacgaggagaccattcaactgttgcctc

SEQ ID NO: 1236 - RNA, 37 nt, synthetic construct
  source 1..37: mol_type = other RNA
attgctccttacgaggagacattcaactgttgcctcc

SEQ ID NO: 1237 - RNA, 37 nt, synthetic construct
  source 1..37: mol_type = other RNA
attgctccttacgaggagacaactgttgcctccggtt

SEQ ID NO: 1238 - RNA, 37 nt, synthetic construct
  source 1..37: mol_type = other RNA
attgctccttacgaggagacagaaccggaggcaacag

SEQ ID NO: 1239 - RNA, 37 nt, synthetic construct
  source 1..37: mol_type = other RNA
attgctccttacgaggagaccctccggttctgaaggt

SEQ ID NO: 1240 - RNA, 37 nt, synthetic construct
  source 1..37: mol_type = other RNA
attgctccttacgaggagactgaaggtgttcttgtac

SEQ ID NO: 1241 - RNA, 37 nt, synthetic construct
  source 1..37: mol_type = other RNA
attgctccttacgaggagacttgtacttcatcccact

SEQ ID NO: 1242 - RNA, 37 nt, synthetic construct
  source 1..37: mol_type = other RNA
attgctccttacgaggagactacttcatcccactgat

SEQ ID NO: 1243 - RNA, 37 nt, synthetic construct
  source 1..37: mol_type = other RNA
attgctccttacgaggagacatcccactgattctgaa

SEQ ID NO: 1244 - RNA, 37 nt, synthetic construct
  source 1..37: mol_type = other RNA
attgctccttacgaggagacagaatcagtgggatgaa

SEQ ID NO: 1245 - RNA, 37 nt, synthetic construct
  source 1..37: mol_type = other RNA
attgctccttacgaggagacttctagcctcttgattg

SEQ ID NO: 1246 - RNA, 37 nt, synthetic construct
  source 1..37: mol_type = other RNA
attgctccttacgaggagactagcctcttgattgctg

SEQ ID NO: 1247 - RNA, 37 nt, synthetic construct
  source 1..37: mol_type = other RNA
attgctccttacgaggagacattgctggtcttgtttt

SEQ ID NO: 1248 - RNA, 37 nt, synthetic construct
  source 1..37: mol_type = other RNA
attgctccttacgaggagacctggtcttgtttttcaa

SEQ ID NO: 1249 - RNA, 37 nt, synthetic construct
  source 1..37: mol_type = other RNA
attgctccttacgaggagacaaaaacaagaccagcaa

SEQ ID NO: 1250 - RNA, 37 nt, synthetic construct
  source 1..37: mol_type = other RNA
attgctccttacgaggagacgaaaaacaagaccagca

SEQ ID NO: 1251 - RNA, 37 nt, synthetic construct
  source 1..37: mol_type = other RNA
attgctccttacgaggagactttttcaaattttgggc

SEQ ID NO: 1252 - RNA, 37 nt, synthetic construct
  source 1..37: mol_type = other RNA
attgctccttacgaggagacttcaaattttgggcagc

SEQ ID NO: 1253 - RNA, 37 nt, synthetic construct
  source 1..37: mol_type = other RNA
attgctccttacgaggagactcaaattttgggcagcg

SEQ ID NO: 1254 - RNA, 37 nt, synthetic construct
  source 1..37: mol_type = other RNA
attgctccttacgaggagaccaaattttgggcagcgg

SEQ ID NO: 1255 - RNA, 37 nt, synthetic construct
  source 1..37: mol_type = other RNA
attgctccttacgaggagacaaattttgggcagcggt

SEQ ID NO: 1256 - RNA, 37 nt, synthetic construct
  source 1..37: mol_type = other RNA
attgctccttacgaggagacccgctgcccaaaatttg

SEQ ID NO: 1257 - RNA, 37 nt, synthetic construct
  source 1..37: mol_type = other RNA
attgctccttacgaggagactgggcagcggtaatgag

SEQ ID NO: 1258 - RNA, 37 nt, synthetic construct
  source 1..37: mol_type = other RNA
attgctccttacgaggagacgggcagcggtaatgagt

SEQ ID NO: 1259 - RNA, 37 nt, synthetic construct
  source 1..37: mol_type = other RNA
attgctccttacgaggagacggcagcggtaatgagtt

SEQ ID NO: 1260 - RNA, 37 nt, synthetic construct
  source 1..37: mol_type = other RNA
attgctccttacgaggagacgaagaactcattaccgc

SEQ ID NO: 1261 - RNA, 37 nt, synthetic construct
  source 1..37: mol_type = other RNA
attgctccttacgaggagacttccaactggggacgcc

SEQ ID NO: 1262 - RNA, 37 nt, synthetic construct
  source 1..37: mol_type = other RNA
attgctccttacgaggagaccaactggggacgcctct

SEQ ID NO: 1263 - RNA, 37 nt, synthetic construct
  source 1..37: mol_type = other RNA
attgctccttacgaggagacgaacagaggcgtcccca

SEQ ID NO: 1264 - RNA, 37 nt, synthetic construct
  source 1..37: mol_type = other RNA
attgctccttacgaggagacggaacagaggcgtcccc

SEQ ID NO: 1265 - RNA, 37 nt, synthetic construct
  source 1..37: mol_type = other RNA
attgctccttacgaggagaccaaatcctgcattgttg

SEQ ID NO: 1266 - RNA, 37 nt, synthetic construct
  source 1..37: mol_type = other RNA
attgctccttacgaggagactgcttgatgatcatctc

SEQ ID NO: 1267 - RNA, 37 nt, synthetic construct
  source 1..37: mol_type = other RNA
attgctccttacgaggagacatgatcatctcgttgat

SEQ ID NO: 1268 - RNA, 37 nt, synthetic construct
  source 1..37: mol_type = other RNA
attgctccttacgaggagacaggatatcaacgagatg

SEQ ID NO: 1269 - RNA, 37 nt, synthetic construct
  source 1..37: mol_type = other RNA
attgctccttacgaggagacatatcctcaaggtcacc

SEQ ID NO: 1270 - RNA, 37 nt, synthetic construct
  source 1..37: mol_type = other RNA
attgctccttacgaggagactaaaatcacagagggtg

SEQ ID NO: 1271 - RNA, 37 nt, synthetic construct
  source 1..37: mol_type = other RNA
attgctccttacgaggagactataacttgatcaagca

SEQ ID NO: 1272 - RNA, 37 nt, synthetic construct
  source 1..37: mol_type = other RNA
attgctccttacgaggagacataacttgatcaagcag

SEQ ID NO: 1273 - RNA, 37 nt, synthetic construct
  source 1..37: mol_type = other RNA
attgctccttacgaggagactaacttgatcaagcaga

SEQ ID NO: 1274 - RNA, 37 nt, synthetic construct
  source 1..37: mol_type = other RNA
attgctccttacgaggagactctgcttgatcaagtta

SEQ ID NO: 1275 - RNA, 37 nt, synthetic construct
  source 1..37: mol_type = other RNA
attgctccttacgaggagacctctgcttgatcaagtt

SEQ ID NO: 1276 - RNA, 37 nt, synthetic construct
  source 1..37: mol_type = other RNA
attgctccttacgaggagacatcaagcagagaaagcc

SEQ ID NO: 1277 - RNA, 37 nt, synthetic construct
  source 1..37: mol_type = other RNA
attgctccttacgaggagacccgactggctttctctg

SEQ ID NO: 1278 - RNA, 37 nt, synthetic construct
  source 1..37: mol_type = other RNA
attgctccttacgaggagacgacagaacttaccgact

SEQ ID NO: 1279 - RNA, 37 nt, synthetic construct
  source 1..37: mol_type = other RNA
attgctccttacgaggagactgtccaagcccggttga

SEQ ID NO: 1280 - RNA, 37 nt, synthetic construct
  source 1..37: mol_type = other RNA
attgctccttacgaggagacaaccgggcttggacaga

SEQ ID NO: 1281 - RNA, 37 nt, synthetic construct
  source 1..37: mol_type = other RNA
attgctccttacgaggagaccaaccgggcttggacag

SEQ ID NO: 1282 - RNA, 37 nt, synthetic construct
  source 1..37: mol_type = other RNA
attgctccttacgaggagacaaatctgccagagcagg

SEQ ID NO: 1283 - RNA, 37 nt, synthetic construct
  source 1..37: mol_type = other RNA
attgctccttacgaggagacgaggtacctgctctggc

SEQ ID NO: 1284 - RNA, 37 nt, synthetic construct
  source 1..37: mol_type = other RNA
attgctccttacgaggagacatgttggaggtacctgc

SEQ ID NO: 1285 - RNA, 37 nt, synthetic construct
  source 1..37: mol_type = other RNA
attgctccttacgaggagaccttgatgttggaggtac

SEQ ID NO: 1286 - RNA, 37 nt, synthetic construct
  source 1..37: mol_type = other RNA
attgctccttacgaggagacctagtttggagatggca

SEQ ID NO: 1287 - RNA, 37 nt, synthetic construct
  source 1..37: mol_type = other RNA
attgctccttacgaggagactagtttggagatggcag

SEQ ID NO: 1288 - RNA, 37 nt, synthetic construct
  source 1..37: mol_type = other RNA
attgctccttacgaggagacggagatggcagtttcct

SEQ ID NO: 1289 - RNA, 37 nt, synthetic construct
  source 1..37: mol_type = other RNA
attgctccttacgaggagacgagatggcagtttcctt

SEQ ID NO: 1290 - RNA, 37 nt, synthetic construct
  source 1..37: mol_type = other RNA
attgctccttacgaggagacctaaggaaactgccatc

SEQ ID NO: 1291 - RNA, 37 nt, synthetic construct
  source 1..37: mol_type = other RNA
attgctccttacgaggagacccttagtaaccacaggt

SEQ ID NO: 1292 - RNA, 37 nt, synthetic construct
  source 1..37: mol_type = other RNA
attgctccttacgaggagaccttagtaaccacaggtt

SEQ ID NO: 1293 - RNA, 37 nt, synthetic construct
  source 1..37: mol_type = other RNA
attgctccttacgaggagacgtaaccacaggttgtgt

SEQ ID NO: 1294 - RNA, 37 nt, synthetic construct
  source 1..37: mol_type = other RNA
attgctccttacgaggagacctctggtgacacaacct

SEQ ID NO: 1295 - RNA, 37 nt, synthetic construct
  source 1..37: mol_type = other RNA
attgctccttacgaggagactgtcaccagagtaacag

SEQ ID NO: 1296 - RNA, 37 nt, synthetic construct
  source 1..37: mol_type = other RNA
attgctccttacgaggagactttctgtctgacagctg

SEQ ID NO: 1297 - RNA, 37 nt, synthetic construct
  source 1..37: mol_type = other RNA
attgctccttacgaggagacttctgtctgacagctgt

SEQ ID NO: 1298 - RNA, 37 nt, synthetic construct
  source 1..37: mol_type = other RNA
attgctccttacgaggagactctgtctgacagctgtt

SEQ ID NO: 1299 - RNA, 37 nt, synthetic construct
  source 1..37: mol_type = other RNA
attgctccttacgaggagacctgtctgacagctgttt

SEQ ID NO: 1300 - RNA, 37 nt, synthetic construct
  source 1..37: mol_type = other RNA
attgctccttacgaggagactgtctgacagctgtttg

SEQ ID NO: 1301 - RNA, 37 nt, synthetic construct
  source 1..37: mol_type = other RNA
attgctccttacgaggagacgcagacctcctgccacc

SEQ ID NO: 1302 - RNA, 37 nt, synthetic construct
  source 1..37: mol_type = other RNA
attgctccttacgaggagaccagacctcctgccaccg

SEQ ID NO: 1303 - RNA, 37 nt, synthetic construct
  source 1..37: mol_type = other RNA
attgctccttacgaggagacggaagcctgaatctgcg

SEQ ID NO: 1304 - RNA, 37 nt, synthetic construct
  source 1..37: mol_type = other RNA
attgctccttacgaggagacaggcttcccaatttttc

SEQ ID NO: 1305 - RNA, 37 nt, synthetic construct
  source 1..37: mol_type = other RNA
attgctccttacgaggagacccaatttttcctgtaga

SEQ ID NO: 1306 - RNA, 37 nt, synthetic construct
  source 1..37: mol_type = other RNA
attgctccttacgaggagactacaggaaaaattggga

SEQ ID NO: 1307 - RNA, 37 nt, synthetic construct
  source 1..37: mol_type = other RNA
attgctccttacgaggagacttcctgtagaatactgg

SEQ ID NO: 1308 - RNA, 37 nt, synthetic construct
  source 1..37: mol_type = other RNA
attgctccttacgaggagactcctgtagaatactggc

SEQ ID NO: 1309 - RNA, 37 nt, synthetic construct
  source 1..37: mol_type = other RNA
attgctccttacgaggagaccctgtagaatactggca

SEQ ID NO: 1310 - RNA, 37 nt, synthetic construct
  source 1..37: mol_type = other RNA
attgctccttacgaggagacctgtagaatactggcat

SEQ ID NO: 1311 - RNA, 37 nt, synthetic construct
  source 1..37: mol_type = other RNA
attgctccttacgaggagacagcaatcctcaaaaaca

SEQ ID NO: 1312 - RNA, 37 nt, synthetic construct
  source 1..37: mol_type = other RNA
attgctccttacgaggagacttgaggattgctgaatt

SEQ ID NO: 1313 - RNA, 37 nt, synthetic construct
  source 1..37: mol_type = other RNA
attgctccttacgaggagactgaggattgctgaatta

SEQ ID NO: 1314 - RNA, 37 nt, synthetic construct
  source 1..37: mol_type = other RNA
attgctccttacgaggagacgaggattgctgaattat

SEQ ID NO: 1315 - RNA, 37 nt, synthetic construct
  source 1..37: mol_type = other RNA
attgctccttacgaggagacaggattgctgaattatt

SEQ ID NO: 1316 - RNA, 37 nt, synthetic construct
  source 1..37: mol_type = other RNA
attgctccttacgaggagacctgaattatttcttccc

SEQ ID NO: 1317 - RNA, 37 nt, synthetic construct
  source 1..37: mol_type = other RNA
attgctccttacgaggagactttcttccccagttgca

SEQ ID NO: 1318 - RNA, 37 nt, synthetic construct
  source 1..37: mol_type = other RNA
attgctccttacgaggagaccttccccagttgcattc

SEQ ID NO: 1319 - RNA, 37 nt, synthetic construct
  source 1..37: mol_type = other RNA
attgctccttacgaggagacttccccagttgcattca

SEQ ID NO: 1320 - RNA, 37 nt, synthetic construct
  source 1..37: mol_type = other RNA
attgctccttacgaggagacaatgcaactggggaaga

SEQ ID NO: 1321 - RNA, 37 nt, synthetic construct
  source 1..37: mol_type = other RNA
attgctccttacgaggagaccccagttgcattcaatg

SEQ ID NO: 1322 - RNA, 37 nt, synthetic construct
  source 1..37: mol_type = other RNA
attgctccttacgaggagaccattcaatgttctgaca

SEQ ID NO: 1323 - RNA, 37 nt, synthetic construct
  source 1..37: mol_type = other RNA
attgctccttacgaggagactcagaacattgaatgca

SEQ ID NO: 1324 - RNA, 37 nt, synthetic construct
  source 1..37: mol_type = other RNA
attgctccttacgaggagacaatgttctgacaacagt

SEQ ID NO: 1325 - RNA, 37 nt, synthetic construct
  source 1..37: mol_type = other RNA
attgctccttacgaggagactgacaacagtttgccgc

SEQ ID NO: 1326 - RNA, 37 nt, synthetic construct
  source 1..37: mol_type = other RNA
attgctccttacgaggagacggcagcggcaaactgtt

SEQ ID NO: 1327 - RNA, 37 nt, synthetic construct
  source 1..37: mol_type = other RNA
attgctccttacgaggagacgccgctgcccaatgcca

SEQ ID NO: 1328 - RNA, 37 nt, synthetic construct
  source 1..37: mol_type = other RNA
attgctccttacgaggagacccgctgcccaatgccat

SEQ ID NO: 1329 - RNA, 37 nt, synthetic construct
  source 1..37: mol_type = other RNA
attgctccttacgaggagacgtatttagcatgttccc

SEQ ID NO: 1330 - RNA, 37 nt, synthetic construct
  source 1..37: mol_type = other RNA
attgctccttacgaggagactatttagcatgttccca

SEQ ID NO: 1331 - RNA, 37 nt, synthetic construct
  source 1..37: mol_type = other RNA
attgctccttacgaggagacggaacatgctaaataca

SEQ ID NO: 1332 - RNA, 37 nt, synthetic construct
  source 1..37: mol_type = other RNA
attgctccttacgaggagacagcatgttcccaattct

SEQ ID NO: 1333 - RNA, 37 nt, synthetic construct
  source 1..37: mol_type = other RNA
attgctccttacgaggagacgcatgttcccaattctc

SEQ ID NO: 1334 - RNA, 37 nt, synthetic construct
  source 1..37: mol_type = other RNA
attgctccttacgaggagacctgagaattgggaacat

SEQ ID NO: 1335 - RNA, 37 nt, synthetic construct
  source 1..37: mol_type = other RNA
attgctccttacgaggagacccaattctcaggaattt

SEQ ID NO: 1336 - RNA, 37 nt, synthetic construct
  source 1..37: mol_type = other RNA
attgctccttacgaggagactcaggaatttgtgtctt

SEQ ID NO: 1337 - RNA, 37 nt, synthetic construct
  source 1..37: mol_type = other RNA
attgctccttacgaggagactcagaaagacacaaatt

SEQ ID NO: 1338 - RNA, 37 nt, synthetic construct
  source 1..37: mol_type = other RNA
attgctccttacgaggagacgtgtctttctgagaaac

SEQ ID NO: 1339 - RNA, 37 nt, synthetic construct
  source 1..37: mol_type = other RNA
attgctccttacgaggagacctcagaaagacacaaat

SEQ ID NO: 1340 - RNA, 37 nt, synthetic construct
  source 1..37: mol_type = other RNA
attgctccttacgaggagactgtctttctgagaaact

SEQ ID NO: 1341 - RNA, 37 nt, synthetic construct
  source 1..37: mol_type = other RNA
attgctccttacgaggagacctgagaaactgttcagc

SEQ ID NO: 1342 - RNA, 37 nt, synthetic construct
  source 1..37: mol_type = other RNA
attgctccttacgaggagactgagaaactgttcagct

SEQ ID NO: 1343 - RNA, 37 nt, synthetic construct
  source 1..37: mol_type = other RNA
attgctccttacgaggagacagcttctgttagccact

SEQ ID NO: 1344 - RNA, 37 nt, synthetic construct
  source 1..37: mol_type = other RNA
attgctccttacgaggagactgttagccactgattaa

SEQ ID NO: 1345 - RNA, 37 nt, synthetic construct
  source 1..37: mol_type = other RNA
attgctccttacgaggagacatcagtggctaacagaa

SEQ ID NO: 1346 - RNA, 37 nt, synthetic construct
  source 1..37: mol_type = other RNA
attgctccttacgaggagacaatcagtggctaacaga

SEQ ID NO: 1347 - RNA, 37 nt, synthetic construct
  source 1..37: mol_type = other RNA
attgctccttacgaggagacgccactgattaaatatc

SEQ ID NO: 1348 - RNA, 37 nt, synthetic construct
  source 1..37: mol_type = other RNA
attgctccttacgaggagactatcataatgaaaacgc

SEQ ID NO: 1349 - RNA, 37 nt, synthetic construct
  source 1..37: mol_type = other RNA
attgctccttacgaggagacagaaatggcggcgtttt

SEQ ID NO: 1350 - RNA, 37 nt, synthetic construct
  source 1..37: mol_type = other RNA
attgctccttacgaggagacctcaacagatctgtcaa

SEQ ID NO: 1351 - RNA, 37 nt, synthetic construct
  source 1..37: mol_type = other RNA
attgctccttacgaggagactcaacagatctgtcaaa

SEQ ID NO: 1352 - RNA, 37 nt, synthetic construct
  source 1..37: mol_type = other RNA
attgctccttacgaggagacacagatctgttgagaaa

SEQ ID NO: 1353 - RNA, 37 nt, synthetic construct
  source 1..37: mol_type = other RNA
attgctccttacgaggagacgacagatctgttgagaa

SEQ ID NO: 1354 - RNA, 38 nt, synthetic construct
  source 1..38: mol_type = other RNA
gattgctccttacgaggagacgtttctgtgattttctt

SEQ ID NO: 1355 - RNA, 38 nt, synthetic construct
  source 1..38: mol_type = other RNA
gattgctccttacgaggagacctgtgattttcttttgg

SEQ ID NO: 1356 - RNA, 38 nt, synthetic construct
  source 1..38: mol_type = other RNA
gattgctccttacgaggagactgtgattttcttttgga

SEQ ID NO: 1357 - RNA, 38 nt, synthetic construct
  source 1..38: mol_type = other RNA
gattgctccttacgaggagactcttttggattgcatct

SEQ ID NO: 1358 - RNA, 38 nt, synthetic construct
  source 1..38: mol_type = other RNA
gattgctccttacgaggagaccttttggattgcatcta

SEQ ID NO: 1359 - RNA, 38 nt, synthetic construct
  source 1..38: mol_type = other RNA
gattgctccttacgaggagacttttggattgcatctac

SEQ ID NO: 1360 - RNA, 38 nt, synthetic construct
  source 1..38: mol_type = other RNA
gattgctccttacgaggagactggattgcatctactgt

SEQ ID NO: 1361 - RNA, 38 nt, synthetic construct
  source 1..38: mol_type = other RNA
gattgctccttacgaggagacggattgcatctactgta

SEQ ID NO: 1362 - RNA, 38 nt, synthetic construct
  source 1..38: mol_type = other RNA
gattgctccttacgaggagacgattgcatctactgtat

SEQ ID NO: 1363 - RNA, 38 nt, synthetic construct
  source 1..38: mol_type = other RNA
gattgctccttacgaggagaccatctactgtataggga

SEQ ID NO: 1364 - RNA, 38 nt, synthetic construct
  source 1..38: mol_type = other RNA
gattgctccttacgaggagacagtcatggaaggagggt

SEQ ID NO: 1365 - RNA, 38 nt, synthetic construct
  source 1..38: mol_type = other RNA
gattgctccttacgaggagaccatgactcaagcttggc

SEQ ID NO: 1366 - RNA, 38 nt, synthetic construct
  source 1..38: mol_type = other RNA
gattgctccttacgaggagacgctctggcctgtcctaa

SEQ ID NO: 1367 - RNA, 38 nt, synthetic construct
  source 1..38: mol_type = other RNA
gattgctccttacgaggagacggacaggccagagccaa

SEQ ID NO: 1368 - RNA, 38 nt, synthetic construct
  source 1..38: mol_type = other RNA
gattgctccttacgaggagacttccttagcttccagcc

SEQ ID NO: 1369 - RNA, 38 nt, synthetic construct
  source 1..38: mol_type = other RNA
gattgctccttacgaggagaccttagcttccagccatt

SEQ ID NO: 1370 - RNA, 38 nt, synthetic construct
  source 1..38: mol_type = other RNA
gattgctccttacgaggagacgcttccagccattgtgt

SEQ ID NO: 1371 - RNA, 38 nt, synthetic construct
  source 1..38: mol_type = other RNA
gattgctccttacgaggagaccagccattgtgttgaat

SEQ ID NO: 1372 - RNA, 38 nt, synthetic construct
  source 1..38: mol_type = other RNA
gattgctccttacgaggagacaacacaatggctggaag

SEQ ID NO: 1373 - RNA, 38 nt, synthetic construct
  source 1..38: mol_type = other RNA
gattgctccttacgaggagacaaggattcaacacaatg

SEQ ID NO: 1374 - RNA, 38 nt, synthetic construct
  source 1..38: mol_type = other RNA
gattgctccttacgaggagactgttgaatcctttaaca

SEQ ID NO: 1375 - RNA, 38 nt, synthetic construct
  source 1..38: mol_type = other RNA
gattgctccttacgaggagacaacatttcattcaactg

SEQ ID NO: 1376 - RNA, 38 nt, synthetic construct
  source 1..38: mol_type = other RNA
gattgctccttacgaggagacacatttcattcaactgt

SEQ ID NO: 1377 - RNA, 38 nt, synthetic construct
  source 1..38: mol_type = other RNA
gattgctccttacgaggagaccattcaactgttgcctc

SEQ ID NO: 1378 - RNA, 38 nt, synthetic construct
  source 1..38: mol_type = other RNA
gattgctccttacgaggagacattcaactgttgcctcc

SEQ ID NO: 1379 - RNA, 38 nt, synthetic construct
  source 1..38: mol_type = other RNA
gattgctccttacgaggagacaactgttgcctccggtt

SEQ ID NO: 1380 - RNA, 38 nt, synthetic construct
  source 1..38: mol_type = other RNA
gattgctccttacgaggagacagaaccggaggcaacag

SEQ ID NO: 1381 - RNA, 38 nt, synthetic construct
  source 1..38: mol_type = other RNA
gattgctccttacgaggagaccctccggttctgaaggt

SEQ ID NO: 1382 - RNA, 38 nt, synthetic construct
  source 1..38: mol_type = other RNA
gattgctccttacgaggagactgaaggtgttcttgtac

SEQ ID NO: 1383 - RNA, 38 nt, synthetic construct
  source 1..38: mol_type = other RNA
gattgctccttacgaggagacttgtacttcatcccact

SEQ ID NO: 1384 - RNA, 38 nt, synthetic construct
  source 1..38: mol_type = other RNA
gattgctccttacgaggagactacttcatcccactgat

SEQ ID NO: 1385 - RNA, 38 nt, synthetic construct
  source 1..38: mol_type = other RNA
gattgctccttacgaggagacatcccactgattctgaa

SEQ ID NO: 1386 - RNA, 38 nt, synthetic construct
  source 1..38: mol_type = other RNA
gattgctccttacgaggagacagaatcagtgggatgaa

SEQ ID NO: 1387 - RNA, 38 nt, synthetic construct
  source 1..38: mol_type = other RNA
gattgctccttacgaggagacttctagcctcttgattg

SEQ ID NO: 1388 - RNA, 38 nt, synthetic construct
  source 1..38: mol_type = other RNA
gattgctccttacgaggagactagcctcttgattgctg

SEQ ID NO: 1389 - RNA, 38 nt, synthetic construct
  source 1..38: mol_type = other RNA
gattgctccttacgaggagacattgctggtcttgtttt

SEQ ID NO: 1390 - RNA, 38 nt, synthetic construct
  source 1..38: mol_type = other RNA
gattgctccttacgaggagacctggtcttgtttttcaa

SEQ ID NO: 1391 - RNA, 38 nt, synthetic construct
  source 1..38: mol_type = other RNA
gattgctccttacgaggagacaaaaacaagaccagcaa

SEQ ID NO: 1392 - RNA, 38 nt, synthetic construct
  source 1..38: mol_type = other RNA
gattgctccttacgaggagacgaaaaacaagaccagca

SEQ ID NO: 1393 - RNA, 38 nt, synthetic construct
  source 1..38: mol_type = other RNA
gattgctccttacgaggagactttttcaaattttgggc

SEQ ID NO: 1394 - RNA, 38 nt, synthetic construct
  source 1..38: mol_type = other RNA
gattgctccttacgaggagacttcaaattttgggcagc

SEQ ID NO: 1395 - RNA, 38 nt, synthetic construct
  source 1..38: mol_type = other RNA
gattgctccttacgaggagactcaaattttgggcagcg

SEQ ID NO: 1396 - RNA, 38 nt, synthetic construct
  source 1..38: mol_type = other RNA
gattgctccttacgaggagaccaaattttgggcagcgg

SEQ ID NO: 1397 - RNA, 38 nt, synthetic construct
  source 1..38: mol_type = other RNA
gattgctccttacgaggagacaaattttgggcagcggt

SEQ ID NO: 1398 - RNA, 38 nt, synthetic construct
  source 1..38: mol_type = other RNA
gattgctccttacgaggagacccgctgcccaaaatttg

SEQ ID NO: 1399 - RNA, 38 nt, synthetic construct
  source 1..38: mol_type = other RNA
gattgctccttacgaggagactgggcagcggtaatgag

SEQ ID NO: 1400 - RNA, 38 nt, synthetic construct
  source 1..38: mol_type = other RNA
gattgctccttacgaggagacgggcagcggtaatgagt

SEQ ID NO: 1401 - RNA, 38 nt, synthetic construct
  source 1..38: mol_type = other RNA
gattgctccttacgaggagacggcagcggtaatgagtt

SEQ ID NO: 1402 - RNA, 38 nt, synthetic construct
  source 1..38: mol_type = other RNA
gattgctccttacgaggagacgaagaactcattaccgc

SEQ ID NO: 1403 - RNA, 38 nt, synthetic construct
  source 1..38: mol_type = other RNA
gattgctccttacgaggagacttccaactggggacgcc

SEQ ID NO: 1404 - RNA, 38 nt, synthetic construct
  source 1..38: mol_type = other RNA
gattgctccttacgaggagaccaactggggacgcctct

SEQ ID NO: 1405 - RNA, 38 nt, synthetic construct
  source 1..38: mol_type = other RNA
gattgctccttacgaggagacgaacagaggcgtcccca

SEQ ID NO: 1406 - RNA, 38 nt, synthetic construct
  source 1..38: mol_type = other RNA
gattgctccttacgaggagacggaacagaggcgtcccc

SEQ ID NO: 1407 - RNA, 38 nt, synthetic construct
  source 1..38: mol_type = other RNA
gattgctccttacgaggagaccaaatcctgcattgttg

SEQ ID NO: 1408 - RNA, 38 nt, synthetic construct
  source 1..38: mol_type = other RNA
gattgctccttacgaggagactgcttgatgatcatctc

SEQ ID NO: 1409 - RNA, 38 nt, synthetic construct
  source 1..38: mol_type = other RNA
gattgctccttacgaggagacatgatcatctcgttgat

SEQ ID NO: 1410 - RNA, 38 nt, synthetic construct
  source 1..38: mol_type = other RNA
gattgctccttacgaggagacaggatatcaacgagatg

SEQ ID NO: 1411 - RNA, 38 nt, synthetic construct
  source 1..38: mol_type = other RNA
gattgctccttacgaggagacatatcctcaaggtcacc

SEQ ID NO: 1412 - RNA, 38 nt, synthetic construct
  source 1..38: mol_type = other RNA
gattgctccttacgaggagactaaaatcacagagggtg

SEQ ID NO: 1413 - RNA, 38 nt, synthetic construct
  source 1..38: mol_type = other RNA
gattgctccttacgaggagactataacttgatcaagca

SEQ ID NO: 1414 - RNA, 38 nt, synthetic construct
  source 1..38: mol_type = other RNA
gattgctccttacgaggagacataacttgatcaagcag

SEQ ID NO: 1415 - RNA, 38 nt, synthetic construct
  source 1..38: mol_type = other RNA
gattgctccttacgaggagactaacttgatcaagcaga

SEQ ID NO: 1416 - RNA, 38 nt, synthetic construct
  source 1..38: mol_type = other RNA
gattgctccttacgaggagactctgcttgatcaagtta

SEQ ID NO: 1417 - RNA, 38 nt, synthetic construct
  source 1..38: mol_type = other RNA
gattgctccttacgaggagacctctgcttgatcaagtt

SEQ ID NO: 1418 - RNA, 38 nt, synthetic construct
  source 1..38: mol_type = other RNA
gattgctccttacgaggagacatcaagcagagaaagcc

SEQ ID NO: 1419 - RNA, 38 nt, synthetic construct
  source 1..38: mol_type = other RNA
gattgctccttacgaggagacccgactggctttctctg

SEQ ID NO: 1420 - RNA, 38 nt, synthetic construct
  source 1..38: mol_type = other RNA
gattgctccttacgaggagacgacagaacttaccgact

SEQ ID NO: 1421 - RNA, 38 nt, synthetic construct
  source 1..38: mol_type = other RNA
gattgctccttacgaggagactgtccaagcccggttga

SEQ ID NO: 1422 - RNA, 38 nt, synthetic construct
  source 1..38: mol_type = other RNA
gattgctccttacgaggagacaaccgggcttggacaga

SEQ ID NO: 1423 - RNA, 38 nt, synthetic construct
  source 1..38: mol_type = other RNA
gattgctccttacgaggagaccaaccgggcttggacag

SEQ ID NO: 1424 - RNA, 38 nt, synthetic construct
  source 1..38: mol_type = other RNA
gattgctccttacgaggagacaaatctgccagagcagg

SEQ ID NO: 1425 - RNA, 38 nt, synthetic construct
  source 1..38: mol_type = other RNA
gattgctccttacgaggagacgaggtacctgctctggc

SEQ ID NO: 1426 - RNA, 38 nt, synthetic construct
  source 1..38: mol_type = other RNA
gattgctccttacgaggagacatgttggaggtacctgc

SEQ ID NO: 1427 - RNA, 38 nt, synthetic construct
  source 1..38: mol_type = other RNA
gattgctccttacgaggagaccttgatgttggaggtac

SEQ ID NO: 1428 - RNA, 38 nt, synthetic construct
  source 1..38: mol_type = other RNA
gattgctccttacgaggagacctagtttggagatggca

SEQ ID NO: 1429 - RNA, 38 nt, synthetic construct
  source 1..38: mol_type = other RNA
gattgctccttacgaggagactagtttggagatggcag

SEQ ID NO: 1430 - RNA, 38 nt, synthetic construct
  source 1..38: mol_type = other RNA
gattgctccttacgaggagacggagatggcagtttcct

SEQ ID NO: 1431 - RNA, 38 nt, synthetic construct
  source 1..38: mol_type = other RNA
gattgctccttacgaggagacgagatggcagtttcctt

SEQ ID NO: 1432 - RNA, 38 nt, synthetic construct
  source 1..38: mol_type = other RNA
gattgctccttacgaggagacctaaggaaactgccatc

SEQ ID NO: 1433 - RNA, 38 nt, synthetic construct
  source 1..38: mol_type = other RNA
gattgctccttacgaggagacccttagtaaccacaggt

SEQ ID NO: 1434 - RNA, 38 nt, synthetic construct
  source 1..38: mol_type = other RNA
gattgctccttacgaggagaccttagtaaccacaggtt

SEQ ID NO: 1435 - RNA, 38 nt, synthetic construct
  source 1..38: mol_type = other RNA
gattgctccttacgaggagacgtaaccacaggttgtgt

SEQ ID NO: 1436 - RNA, 38 nt, synthetic construct
  source 1..38: mol_type = other RNA
gattgctccttacgaggagacctctggtgacacaacct

SEQ ID NO: 1437 - RNA, 38 nt, synthetic construct
  source 1..38: mol_type = other RNA
gattgctccttacgaggagactgtcaccagagtaacag

SEQ ID NO: 1438 - RNA, 38 nt, synthetic construct
  source 1..38: mol_type = other RNA
gattgctccttacgaggagactttctgtctgacagctg

SEQ ID NO: 1439 - RNA, 38 nt, synthetic construct
  source 1..38: mol_type = other RNA
gattgctccttacgaggagacttctgtctgacagctgt

SEQ ID NO: 1440 - RNA, 38 nt, synthetic construct
  source 1..38: mol_type = other RNA
gattgctccttacgaggagactctgtctgacagctgtt

SEQ ID NO: 1441 - RNA, 38 nt, synthetic construct
  source 1..38: mol_type = other RNA
gattgctccttacgaggagacctgtctgacagctgttt

SEQ ID NO: 1442 - RNA, 38 nt, synthetic construct
  source 1..38: mol_type = other RNA
gattgctccttacgaggagactgtctgacagctgtttg

SEQ ID NO: 1443 - RNA, 38 nt, synthetic construct
  source 1..38: mol_type = other RNA
gattgctccttacgaggagacgcagacctcctgccacc

SEQ ID NO: 1444 - RNA, 38 nt, synthetic construct
  source 1..38: mol_type = other RNA
gattgctccttacgaggagaccagacctcctgccaccg

SEQ ID NO: 1445 - RNA, 38 nt, synthetic construct
  source 1..38: mol_type = other RNA
gattgctccttacgaggagacggaagcctgaatctgcg

SEQ ID NO: 1446 - RNA, 38 nt, synthetic construct
  source 1..38: mol_type = other RNA
gattgctccttacgaggagacaggcttcccaatttttc

SEQ ID NO: 1447 - RNA, 38 nt, synthetic construct
  source 1..38: mol_type = other RNA
gattgctccttacgaggagacccaatttttcctgtaga

SEQ ID NO: 1448 - RNA, 38 nt, synthetic construct
  source 1..38: mol_type = other RNA
gattgctccttacgaggagactacaggaaaaattggga

SEQ ID NO: 1449 - RNA, 38 nt, synthetic construct
  source 1..38: mol_type = other RNA
gattgctccttacgaggagacttcctgtagaatactgg

SEQ ID NO: 1450 - RNA, 38 nt, synthetic construct
  source 1..38: mol_type = other RNA
gattgctccttacgaggagactcctgtagaatactggc

SEQ ID NO: 1451 - RNA, 38 nt, synthetic construct
  source 1..38: mol_type = other RNA
gattgctccttacgaggagaccctgtagaatactggca

SEQ ID NO: 1452 - RNA, 38 nt, synthetic construct
  source 1..38: mol_type = other RNA
gattgctccttacgaggagacctgtagaatactggcat

SEQ ID NO: 1453 - RNA, 38 nt, synthetic construct
  source 1..38: mol_type = other RNA
gattgctccttacgaggagacagcaatcctcaaaaaca

SEQ ID NO: 1454 - RNA, 38 nt, synthetic construct
  source 1..38: mol_type = other RNA
gattgctccttacgaggagacttgaggattgctgaatt

SEQ ID NO: 1455 - RNA, 38 nt, synthetic construct
  source 1..38: mol_type = other RNA
gattgctccttacgaggagactgaggattgctgaatta

SEQ ID NO: 1456 - RNA, 38 nt, synthetic construct
  source 1..38: mol_type = other RNA
gattgctccttacgaggagacgaggattgctgaattat

SEQ ID NO: 1457 - RNA, 38 nt, synthetic construct
  source 1..38: mol_type = other RNA
gattgctccttacgaggagacaggattgctgaattatt

SEQ ID NO: 1458 - RNA, 38 nt, synthetic construct
  source 1..38: mol_type = other RNA
gattgctccttacgaggagacctgaattatttcttccc

SEQ ID NO: 1459 - RNA, 38 nt, synthetic construct
  source 1..38: mol_type = other RNA
gattgctccttacgaggagactttcttccccagttgca

SEQ ID NO: 1460 - RNA, 38 nt, synthetic construct
  source 1..38: mol_type = other RNA
gattgctccttacgaggagaccttccccagttgcattc

SEQ ID NO: 1461 - RNA, 38 nt, synthetic construct
  source 1..38: mol_type = other RNA
gattgctccttacgaggagacttccccagttgcattca

SEQ ID NO: 1462 - RNA, 38 nt, synthetic construct
  source 1..38: mol_type = other RNA
gattgctccttacgaggagacaatgcaactggggaaga

SEQ ID NO: 1463 - RNA, 38 nt, synthetic construct
  source 1..38: mol_type = other RNA
gattgctccttacgaggagaccccagttgcattcaatg

SEQ ID NO: 1464 - RNA, 38 nt, synthetic construct
  source 1..38: mol_type = other RNA
gattgctccttacgaggagaccattcaatgttctgaca

SEQ ID NO: 1465 - RNA, 38 nt, synthetic construct
  source 1..38: mol_type = other RNA
gattgctccttacgaggagactcagaacattgaatgca

SEQ ID NO: 1466 - RNA, 38 nt, synthetic construct
  source 1..38: mol_type = other RNA
gattgctccttacgaggagacaatgttctgacaacagt

SEQ ID NO: 1467 - RNA, 38 nt, synthetic construct
  source 1..38: mol_type = other RNA
gattgctccttacgaggagactgacaacagtttgccgc

SEQ ID NO: 1468 - RNA, 38 nt, synthetic construct
  source 1..38: mol_type = other RNA
gattgctccttacgaggagacggcagcggcaaactgtt

SEQ ID NO: 1469 - RNA, 38 nt, synthetic construct
  source 1..38: mol_type = other RNA
gattgctccttacgaggagacgccgctgcccaatgcca

SEQ ID NO: 1470 - RNA, 38 nt, synthetic construct
  source 1..38: mol_type = other RNA
gattgctccttacgaggagacccgctgcccaatgccat

SEQ ID NO: 1471 - RNA, 38 nt, synthetic construct
  source 1..38: mol_type = other RNA
gattgctccttacgaggagacgtatttagcatgttccc

SEQ ID NO: 1472 - RNA, 38 nt, synthetic construct
  source 1..38: mol_type = other RNA
gattgctccttacgaggagactatttagcatgttccca

SEQ ID NO: 1473 - RNA, 38 nt, synthetic construct
  source 1..38: mol_type = other RNA
gattgctccttacgaggagacggaacatgctaaataca

SEQ ID NO: 1474 - RNA, 38 nt, synthetic construct
  source 1..38: mol_type = other RNA
gattgctccttacgaggagacagcatgttcccaattct

SEQ ID NO: 1475 - RNA, 38 nt, synthetic construct
  source 1..38: mol_type = other RNA
gattgctccttacgaggagacgcatgttcccaattctc

SEQ ID NO: 1476 - RNA, 38 nt, synthetic construct
  source 1..38: mol_type = other RNA
gattgctccttacgaggagacctgagaattgggaacat

SEQ ID NO: 1477 - RNA, 38 nt, synthetic construct
  source 1..38: mol_type = other RNA
gattgctccttacgaggagacccaattctcaggaattt

SEQ ID NO: 1478 - RNA, 38 nt, synthetic construct
  source 1..38: mol_type = other RNA
gattgctccttacgaggagactcaggaatttgtgtctt

SEQ ID NO: 1479 - RNA, 38 nt, synthetic construct
  source 1..38: mol_type = other RNA
gattgctccttacgaggagactcagaaagacacaaatt

SEQ ID NO: 1480 - RNA, 38 nt, synthetic construct
  source 1..38: mol_type = other RNA
gattgctccttacgaggagacgtgtctttctgagaaac

SEQ ID NO: 1481 - RNA, 38 nt, synthetic construct
  source 1..38: mol_type = other RNA
gattgctccttacgaggagacctcagaaagacacaaat

SEQ ID NO: 1482 - RNA, 38 nt, synthetic construct
  source 1..38: mol_type = other RNA
gattgctccttacgaggagactgtctttctgagaaact

SEQ ID NO: 1483 - RNA, 38 nt, synthetic construct
  source 1..38: mol_type = other RNA
gattgctccttacgaggagacctgagaaactgttcagc

SEQ ID NO: 1484 - RNA, 38 nt, synthetic construct
  source 1..38: mol_type = other RNA
gattgctccttacgaggagactgagaaactgttcagct

SEQ ID NO: 1485 - RNA, 38 nt, synthetic construct
  source 1..38: mol_type = other RNA
gattgctccttacgaggagacagcttctgttagccact

SEQ ID NO: 1486 - RNA, 38 nt, synthetic construct
  source 1..38: mol_type = other RNA
gattgctccttacgaggagactgttagccactgattaa

SEQ ID NO: 1487 - RNA, 38 nt, synthetic construct
  source 1..38: mol_type = other RNA
gattgctccttacgaggagacatcagtggctaacagaa

SEQ ID NO: 1488 - RNA, 38 nt, synthetic construct
  source 1..38: mol_type = other RNA
gattgctccttacgaggagacaatcagtggctaacaga

SEQ ID NO: 1489 - RNA, 38 nt, synthetic construct
  source 1..38: mol_type = other RNA
gattgctccttacgaggagacgccactgattaaatatc

SEQ ID NO: 1490 - RNA, 38 nt, synthetic construct
  source 1..38: mol_type = other RNA
gattgctccttacgaggagactatcataatgaaaacgc

SEQ ID NO: 1491 - RNA, 38 nt, synthetic construct
  source 1..38: mol_type = other RNA
gattgctccttacgaggagacagaaatggcggcgtttt

SEQ ID NO: 1492 - RNA, 38 nt, synthetic construct
  source 1..38: mol_type = other RNA
gattgctccttacgaggagacctcaacagatctgtcaa

SEQ ID NO: 1493 - RNA, 38 nt, synthetic construct
  source 1..38: mol_type = other RNA
gattgctccttacgaggagactcaacagatctgtcaaa

SEQ ID NO: 1494 - RNA, 38 nt, synthetic construct
  source 1..38: mol_type = other RNA
gattgctccttacgaggagacacagatctgttgagaaa

SEQ ID NO: 1495 - RNA, 38 nt, synthetic construct
  source 1..38: mol_type = other RNA
gattgctccttacgaggagacgacagatctgttgagaa

SEQ ID NO: 1496 - RNA, 15 nt, synthetic construct
  source 1..15: mol_type = other RNA
ctgttgcctccggtt

SEQ ID NO: 1497 - RNA, 15 nt, synthetic construct
  source 1..15: mol_type = other RNA
ttgctggtcttgttt

SEQ ID NO: 1498 - RNA, 15 nt, synthetic construct
  source 1..15: mol_type = other RNA
tgctggtcttgtttt

SEQ ID NO: 1499 - RNA, 15 nt, synthetic construct
  source 1..15: mol_type = other RNA
gctgcccaaaatttg

SEQ ID NO: 1500 - RNA, 15 nt, synthetic construct
  source 1..15: mol_type = other RNA
cgctgcccaaaattt

SEQ ID NO: 1501 - RNA, 15 nt, synthetic construct
  source 1..15: mol_type = other RNA
ccgctgcccaaaatt

SEQ ID NO: 1502 - RNA, 15 nt, synthetic construct
  source 1..15: mol_type = other RNA
accgctgcccaaaat

SEQ ID NO: 1503 - RNA, 15 nt, synthetic construct
  source 1..15: mol_type = other RNA
caaattttgggcagc

SEQ ID NO: 1504 - RNA, 15 nt, synthetic construct
  source 1..15: mol_type = other RNA
ctcattaccgctgcc

SEQ ID NO: 1505 - RNA, 15 nt, synthetic construct
  source 1..15: mol_type = other RNA
actcattaccgctgc

SEQ ID NO: 1506 - RNA, 15 nt, synthetic construct
  source 1..15: mol_type = other RNA
aactcattaccgctg

SEQ ID NO: 1507 - RNA, 15 nt, synthetic construct
  source 1..15: mol_type = other RNA
gcggtaatgagttct

SEQ ID NO: 1508 - RNA, 15 nt, synthetic construct
  source 1..15: mol_type = other RNA
ggcgtccccagttgg

SEQ ID NO: 1509 - RNA, 15 nt, synthetic construct
  source 1..15: mol_type = other RNA
agaggcgtccccagt

SEQ ID NO: 1510 - RNA, 15 nt, synthetic construct
  source 1..15: mol_type = other RNA
tggggacgcctctgt

SEQ ID NO: 1511 - RNA, 15 nt, synthetic construct
  source 1..15: mol_type = other RNA
ggggacgcctctgtt

SEQ ID NO: 1512 - RNA, 15 nt, synthetic construct
  source 1..15: mol_type = other RNA
caacaatgcaggatt

SEQ ID NO: 1513 - RNA, 15 nt, synthetic construct
  source 1..15: mol_type = other RNA
aggaaactgccatct

SEQ ID NO: 1514 - RNA, 15 nt, synthetic construct
  source 1..15: mol_type = other RNA
aaggaaactgccatc

SEQ ID NO: 1515 - RNA, 15 nt, synthetic construct
  source 1..15: mol_type = other RNA
ctgttactctggtga

SEQ ID NO: 1516 - RNA, 15 nt, synthetic construct
  source 1..15: mol_type = other RNA
tgaatgcaactgggg

SEQ ID NO: 1517 - RNA, 15 nt, synthetic construct
  source 1..15: mol_type = other RNA
gcggcaaactgttgt

SEQ ID NO: 1518 - RNA, 15 nt, synthetic construct
  source 1..15: mol_type = other RNA
tggcattgggcagcg

SEQ ID NO: 1519 - RNA, 15 nt, synthetic construct
  source 1..15: mol_type = other RNA
atggcattgggcagc

SEQ ID NO: 1520 - RNA, 20 nt, synthetic construct
  source 1..20: mol_type = other RNA
ttaacatttcattcaagtac

SEQ ID NO: 1521 - RNA, 20 nt, synthetic construct
  source 1..20: mol_type = other RNA
gttctagcctcttgaggtac

SEQ ID NO: 1522 - RNA, 20 nt, synthetic construct
  source 1..20: mol_type = other RNA
ttctagcctcttgatggtac

SEQ ID NO: 1523 - RNA, 20 nt, synthetic construct
  source 1..20: mol_type = other RNA
gaagaactcattaccggtac

SEQ ID NO: 1524 - RNA, 20 nt, synthetic construct
  source 1..20: mol_type = other RNA
ggaagaactcattacggtac

SEQ ID NO: 1525 - RNA, 20 nt, synthetic construct
  source 1..20: mol_type = other RNA
tggaagaactcattaggtac

SEQ ID NO: 1526 - RNA, 20 nt, synthetic construct
  source 1..20: mol_type = other RNA
ttggaagaactcattggtac

SEQ ID NO: 1527 - RNA, 20 nt, synthetic construct
  source 1..20: mol_type = other RNA
gctggtcttgtttttggtac

SEQ ID NO: 1528 - RNA, 20 nt, synthetic construct
  source 1..20: mol_type = other RNA
ccccagttggaagaaggtac

SEQ ID NO: 1529 - RNA, 20 nt, synthetic construct
  source 1..20: mol_type = other RNA
tccccagttggaagaggtac

SEQ ID NO: 1530 - RNA, 20 nt, synthetic construct
  source 1..20: mol_type = other RNA
gtccccagttggaagggtac

SEQ ID NO: 1531 - RNA, 20 nt, synthetic construct
  source 1..20: mol_type = other RNA
ttcaaattttgggcaggtac

SEQ ID NO: 1532 - RNA, 20 nt, synthetic construct
  source 1..20: mol_type = other RNA
aggatttggaacagaggtac

SEQ ID NO: 1533 - RNA, 20 nt, synthetic construct
  source 1..20: mol_type = other RNA
tgcaggatttggaacggtac

SEQ ID NO: 1534 - RNA, 20 nt, synthetic construct
  source 1..20: mol_type = other RNA
atgagttcttccaacggtac

SEQ ID NO: 1535 - RNA, 20 nt, synthetic construct
  source 1..20: mol_type = other RNA
tgagttcttccaactggtac

SEQ ID NO: 1536 - RNA, 20 nt, synthetic construct
  source 1..20: mol_type = other RNA
atttgttcttacaggggtac

SEQ ID NO: 1537 - RNA, 20 nt, synthetic construct
  source 1..20: mol_type = other RNA
caacctgtggttactagtac

SEQ ID NO: 1538 - RNA, 20 nt, synthetic construct
  source 1..20: mol_type = other RNA
acaacctgtggttactgtac

SEQ ID NO: 1539 - RNA, 20 nt, synthetic construct
  source 1..20: mol_type = other RNA
ttagctcctactcagagtac

SEQ ID NO: 1540 - RNA, 20 nt, synthetic construct
  source 1..20: mol_type = other RNA
actgttgtcagaacatgtac

SEQ ID NO: 1541 - RNA, 20 nt, synthetic construct
  source 1..20: mol_type = other RNA
aggatggcattgggcagtac

SEQ ID NO: 1542 - RNA, 20 nt, synthetic construct
  source 1..20: mol_type = other RNA
tacaggaactccaggagtac

SEQ ID NO: 1543 - RNA, 20 nt, synthetic construct
  source 1..20: mol_type = other RNA
ttacaggaactccagggtac

SEQ ID NO: 1544 - RNA, 35 nt, synthetic construct
  source 1..35: mol_type = other RNA
ttaacatttcattcaagtacctgttgcctccggtt

SEQ ID NO: 1545 - RNA, 35 nt, synthetic construct
  source 1..35: mol_type = other RNA
gttctagcctcttgaggtacttgctggtcttgttt

SEQ ID NO: 1546 - RNA, 35 nt, synthetic construct
  source 1..35: mol_type = other RNA
ttctagcctcttgatggtactgctggtcttgtttt

SEQ ID NO: 1547 - RNA, 35 nt, synthetic construct
  source 1..35: mol_type = other RNA
gaagaactcattaccggtacgctgcccaaaatttg

SEQ ID NO: 1548 - RNA, 35 nt, synthetic construct
  source 1..35: mol_type = other RNA
ggaagaactcattacggtaccgctgcccaaaattt

SEQ ID NO: 1549 - RNA, 35 nt, synthetic construct
  source 1..35: mol_type = other RNA
tggaagaactcattaggtacccgctgcccaaaatt

SEQ ID NO: 1550 - RNA, 35 nt, synthetic construct
  source 1..35: mol_type = other RNA
ttggaagaactcattggtacaccgctgcccaaaat

SEQ ID NO: 1551 - RNA, 35 nt, synthetic construct
  source 1..35: mol_type = other RNA
gctggtcttgtttttggtaccaaattttgggcagc

SEQ ID NO: 1552 - RNA, 35 nt, synthetic construct
  source 1..35: mol_type = other RNA
ccccagttggaagaaggtacctcattaccgctgcc

SEQ ID NO: 1553 - RNA, 35 nt, synthetic construct
  source 1..35: mol_type = other RNA
tccccagttggaagaggtacactcattaccgctgc

SEQ ID NO: 1554 - RNA, 35 nt, synthetic construct
  source 1..35: mol_type = other RNA
gtccccagttggaagggtacaactcattaccgctg

SEQ ID NO: 1555 - RNA, 35 nt, synthetic construct
  source 1..35: mol_type = other RNA
ttcaaattttgggcaggtacgcggtaatgagttct

SEQ ID NO: 1556 - RNA, 35 nt, synthetic construct
  source 1..35: mol_type = other RNA
aggatttggaacagaggtacggcgtccccagttgg

SEQ ID NO: 1557 - RNA, 35 nt, synthetic construct
  source 1..35: mol_type = other RNA
tgcaggatttggaacggtacagaggcgtccccagt

SEQ ID NO: 1558 - RNA, 35 nt, synthetic construct
  source 1..35: mol_type = other RNA
atgagttcttccaacggtactggggacgcctctgt

SEQ ID NO: 1559 - RNA, 35 nt, synthetic construct
  source 1..35: mol_type = other RNA
tgagttcttccaactggtacggggacgcctctgtt

SEQ ID NO: 1560 - RNA, 35 nt, synthetic construct
  source 1..35: mol_type = other RNA
atttgttcttacaggggtaccaacaatgcaggatt

SEQ ID NO: 1561 - RNA, 35 nt, synthetic construct
  source 1..35: mol_type = other RNA
caacctgtggttactagtacaggaaactgccatct

SEQ ID NO: 1562 - RNA, 35 nt, synthetic construct
  source 1..35: mol_type = other RNA
acaacctgtggttactgtacaaggaaactgccatc

SEQ ID NO: 1563 - RNA, 35 nt, synthetic construct
  source 1..35: mol_type = other RNA
ttagctcctactcagagtacctgttactctggtga

SEQ ID NO: 1564 - RNA, 35 nt, synthetic construct
  source 1..35: mol_type = other RNA
actgttgtcagaacatgtactgaatgcaactgggg

SEQ ID NO: 1565 - RNA, 35 nt, synthetic construct
  source 1..35: mol_type = other RNA
aggatggcattgggcagtacgcggcaaactgttgt

SEQ ID NO: 1566 - RNA, 35 nt, synthetic construct
  source 1..35: mol_type = other RNA
tacaggaactccaggagtactggcattgggcagcg

SEQ ID NO: 1567 - RNA, 35 nt, synthetic construct
  source 1..35: mol_type = other RNA
ttacaggaactccagggtacatggcattgggcagc

SEQ ID NO: 1568 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttaacatttcattcaagtacctgttgcctccggttaaattaacagtggccgcggtcggcgtggactgtagaacactgccaatgccggtcccaagcccggataaaagtggagggtacagtccacgctctagagcggacttcggtccgc

SEQ ID NO: 1569 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgttctagcctcttgaggtacttgctggtcttgtttaaattaacagtggccgcggtcggcgtggactgtagaacactgccaatgccggtcccaagcccggataaaagtggagggtacagtccacgctctagagcggacttcggtccgc

SEQ ID NO: 1570 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttctagcctcttgatggtactgctggtcttgttttaaattaacagtggccgcggtcggcgtggactgtagaacactgccaatgccggtcccaagcccggataaaagtggagggtacagtccacgctctagagcggacttcggtccgc

SEQ ID NO: 1571 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgaagaactcattaccggtacgctgcccaaaatttgaaattaacagtggccgcggtcggcgtggactgtagaacactgccaatgccggtcccaagcccggataaaagtggagggtacagtccacgctctagagcggacttcggtccgc

SEQ ID NO: 1572 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcggaagaactcattacggtaccgctgcccaaaatttaaattaacagtggccgcggtcggcgtggactgtagaacactgccaatgccggtcccaagcccggataaaagtggagggtacagtccacgctctagagcggacttcggtccgc

SEQ ID NO: 1573 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctggaagaactcattaggtacccgctgcccaaaattaaattaacagtggccgcggtcggcgtggactgtagaacactgccaatgccggtcccaagcccggataaaagtggagggtacagtccacgctctagagcggacttcggtccgc

SEQ ID NO: 1574 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttggaagaactcattggtacaccgctgcccaaaataaattaacagtggccgcggtcggcgtggactgtagaacactgccaatgccggtcccaagcccggataaaagtggagggtacagtccacgctctagagcggacttcggtccgc

SEQ ID NO: 1575 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgctggtcttgtttttggtaccaaattttgggcagcaaattaacagtggccgcggtcggcgtggactgtagaacactgccaatgccggtcccaagcccggataaaagtggagggtacagtccacgctctagagcggacttcggtccgc

SEQ ID NO: 1576 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcccccagttggaagaaggtacctcattaccgctgccaaattaacagtggccgcggtcggcgtggactgtagaacactgccaatgccggtcccaagcccggataaaagtggagggtacagtccacgctctagagcggacttcggtccgc

SEQ ID NO: 1577 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctccccagttggaagaggtacactcattaccgctgcaaattaacagtggccgcggtcggcgtggactgtagaacactgccaatgccggtcccaagcccggataaaagtggagggtacagtccacgctctagagcggacttcggtccgc

SEQ ID NO: 1578 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgtccccagttggaagggtacaactcattaccgctgaaattaacagtggccgcggtcggcgtggactgtagaacactgccaatgccggtcccaagcccggataaaagtggagggtacagtccacgctctagagcggacttcggtccgc

SEQ ID NO: 1579 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttcaaattttgggcaggtacgcggtaatgagttctaaattaacagtggccgcggtcggcgtggactgtagaacactgccaatgccggtcccaagcccggataaaagtggagggtacagtccacgctctagagcggacttcggtccgc

SEQ ID NO: 1580 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcaggatttggaacagaggtacggcgtccccagttggaaattaacagtggccgcggtcggcgtggactgtagaacactgccaatgccggtcccaagcccggataaaagtggagggtacagtccacgctctagagcggacttcggtccgc

SEQ ID NO: 1581 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctgcaggatttggaacggtacagaggcgtccccagtaaattaacagtggccgcggtcggcgtggactgtagaacactgccaatgccggtcccaagcccggataaaagtggagggtacagtccacgctctagagcggacttcggtccgc

SEQ ID NO: 1582 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcatgagttcttccaacggtactggggacgcctctgtaaattaacagtggccgcggtcggcgtggactgtagaacactgccaatgccggtcccaagcccggataaaagtggagggtacagtccacgctctagagcggacttcggtccgc

SEQ ID NO: 1583 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctgagttcttccaactggtacggggacgcctctgttaaattaacagtggccgcggtcggcgtggactgtagaacactgccaatgccggtcccaagcccggataaaagtggagggtacagtccacgctctagagcggacttcggtccgc

SEQ ID NO: 1584 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcatttgttcttacaggggtaccaacaatgcaggattaaattaacagtggccgcggtcggcgtggactgtagaacactgccaatgccggtcccaagcccggataaaagtggagggtacagtccacgctctagagcggacttcggtccgc

SEQ ID NO: 1585 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccaacctgtggttactagtacaggaaactgccatctaaattaacagtggccgcggtcggcgtggactgtagaacactgccaatgccggtcccaagcccggataaaagtggagggtacagtccacgctctagagcggacttcggtccgc

SEQ ID NO: 1586 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcacaacctgtggttactgtacaaggaaactgccatcaaattaacagtggccgcggtcggcgtggactgtagaacactgccaatgccggtcccaagcccggataaaagtggagggtacagtccacgctctagagcggacttcggtccgc

SEQ ID NO: 1587 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttagctcctactcagagtacctgttactctggtgaaaattaacagtggccgcggtcggcgtggactgtagaacactgccaatgccggtcccaagcccggataaaagtggagggtacagtccacgctctagagcggacttcggtccgc

SEQ ID NO: 1588 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcactgttgtcagaacatgtactgaatgcaactggggaaattaacagtggccgcggtcggcgtggactgtagaacactgccaatgccggtcccaagcccggataaaagtggagggtacagtccacgctctagagcggacttcggtccgc

SEQ ID NO: 1589 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcaggatggcattgggcagtacgcggcaaactgttgtaaattaacagtggccgcggtcggcgtggactgtagaacactgccaatgccggtcccaagcccggataaaagtggagggtacagtccacgctctagagcggacttcggtccgc

SEQ ID NO: 1590 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctacaggaactccaggagtactggcattgggcagcgaaattaacagtggccgcggtcggcgtggactgtagaacactgccaatgccggtcccaagcccggataaaagtggagggtacagtccacgctctagagcggacttcggtccgc

SEQ ID NO: 1591 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttacaggaactccagggtacatggcattgggcagcaaattaacagtggccgcggtcggcgtggactgtagaacactgccaatgccggtcccaagcccggataaaagtggagggtacagtccacgctctagagcggacttcggtccgc

SEQ ID NO: 1592 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 1593 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 1594 - protein, 1367 aa, synthetic construct; identical to SEQ ID NO: 90

SEQ ID NO: 1595 - protein, 50 aa, synthetic construct
GSSGGSPAGSPTSTEEGTSESATPESGPGTSTEPSEGSAPGSPAGSGGGS

SEQ ID NO: 1596 - RNA, 20 nt, synthetic construct
  source 1..20: mol_type = other RNA
caccgcagccacgcagagca

SEQ ID NO: 1597 - RNA, 17 nt, synthetic construct
  source 1..17: mol_type = other RNA
gagcaacggcggaaggt

SEQ ID NO: 1598 - RNA, 20 nt, synthetic construct
  source 1..20: mol_type = other RNA
tagaaccttgatgacatagc

SEQ ID NO: 1599 - DNA, 452 bp, synthetic construct
  source 1..452: mol_type = other DNA
cgctccggtgcccgttgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagca

SEQ ID NO: 1600 - DNA, 409 bp, synthetic construct
  source 1..409: mol_type = other DNA
ggtaccgctccggtgcccgtcgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcaccggt

SEQ ID NO: 1601 - DNA, 249 bp, synthetic construct
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 1602 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 1603 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 1604 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttggccttcgccctcagacgagtcggatctccctttgggccgcctccccgc

SEQ ID NO: 1605 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 1606 - DNA, 510 bp, synthetic construct
  source 1..510: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaacacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 1607 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 1608 - protein, 837 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 1609 - protein, 692 aa, unidentified
  source 1..692: note = MMLV RT
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFES

SEQ ID NO: 1610 - protein, 1401 aa, synthetic construct
AATMKRTADGSEFESPKKKRKVALDFLSRLPLPPPVSPICTFVSPAAQKAFQPPRSCGSATPGSSRMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKKRPAATKKAGQAKKKKSRKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLDGSG

SEQ ID NO: 1611 - protein, 1420 aa, synthetic construct
AATMKRTADGSEFESPKKKRKVALDFLSRLPLPPPVSPICTFVSPAAQKAFQPPRSCGSATPGSSRMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKKRPAATKKAGQAKKKKSRATNFSLLKQAGDVEENPGPKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLDGSG

SEQ ID NO: 1612 - protein, 1401 aa, synthetic construct; identical to SEQ ID NO: 1610

SEQ ID NO: 1613 - protein, 1440 aa, synthetic construct
AATMKRTADGSEFESPKKKRKVALDFLSRLPLPPPVSPICTFVSPAAQKAFQPPRSCGGSGGSPAGSPTSTEEGTSESATPGSGSRMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKKRPAATKKAGQAKKKKSRATNFSLLKQAGDVEENPGPKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLDGSG

SEQ ID NO: 1614 - protein, 1401 aa, synthetic construct
AATMKRTADGSEFESPKKKRKVSRMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKSATPGSALDFLSRLPLPPPVSPICTFVSPAAQKAFQPPRSCGKRPAATKKAGQAKKKKSRKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLDGSG

SEQ ID NO: 1615 - protein, 1420 aa, synthetic construct
AATMKRTADGSEFESPKKKRKVSRMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKSATPGSALDFLSRLPLPPPVSPICTFVSPAAQKAFQPPRSCGKRPAATKKAGQAKKKKSRATNFSLLKQAGDVEENPGPKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLDGSG

SEQ ID NO: 1616 - protein, 1359 aa, synthetic construct
AATMKRTADGSEFESPKKKRKVSRMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKKRPAATKKAGQAKKKKSRKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLDGSG

SEQ ID NO: 1617 - protein, 1401 aa, synthetic construct
AATMKRTADGSEFESPKKKRKVSRMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKKRPAATKKAGQAKKKKSRKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESSATPGSALDFLSRLPLPPPVSPICTFVSPAAQKAFQPPRSCGPKKKRKVGSGPAAKRVKLDGSG

SEQ ID NO: 1618 - protein, 1378 aa, synthetic construct
AATMKRTADGSEFESPKKKRKVSRMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKKRPAATKKAGQAKKKKSRATNFSLLKQAGDVEENPGPKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLDGSG

SEQ ID NO: 1619 - protein, 1401 aa, synthetic construct; identical to SEQ ID NO: 1617

SEQ ID NO: 1620 - RNA, 89 nt, synthetic construct
  source 1..89: mol_type = other RNA
acagcttatttggaagctgaaatgtgaggtttataacactcacaagaatcctgaaaaaggatgccaaacttccaaatcctgcattgttg

SEQ ID NO: 1621 - RNA, 89 nt, synthetic construct
  source 1..89: mol_type = other RNA
acagcttatttggaagctgaaatgtgaggtttataacactcacaagaatcctgaaaaaggatgccaaacgaagaactcattaccgctgc

SEQ ID NO: 1622 - RNA, 323 nt, synthetic construct
  source 1..323: mol_type = other RNA
tgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctaagggatatttgttcttacaggcaacaatgcattaccgctgcccaaaatttgaaaaacaagaccagcaatcaaattaacagtggccgcggtcggcgtggactgtagaacactgccaatgccggtcccaagcccggataaaagtggagggtacagtccacgctctagagcggacttcggtccgc

SEQ ID NO: 1623 - RNA, 323 nt, synthetic construct
  source 1..323: mol_type = other RNA
tgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgattgctggtcttgtttttcaaattttgggcagcggtaatgcattgttgcctgtaagaacaaatatcccttaaaattaacagtggccgcggtcggcgtggactgtagaacactgccaatgccggtcccaagcccggataaaagtggagggtacagtccacgctctagagcggacttcggtccgc

SEQ ID NO: 1624 - RNA, 353 nt, synthetic construct
  source 1..353: mol_type = other RNA
tgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcacagaggcgtccccagtacgaagaactcattaccggtaccctgcccaaaatttgaaaaacaagaccagcaatcaagaggctagaacaatcattacggatcgaaaattaacagtggccgcggtcggcgtggactgtagaacactgccaatgccggtcccaagcccggataaaagtggagggtacagtccacgctctagagcggacttcggtccgc

SEQ ID NO: 1625 - protein, 36 aa, synthetic construct
ALDFLSRLPLPPPVSPICTFVSPAAQKAFQPPRSCG

SEQ ID NO: 1626 - protein, 129 aa, synthetic construct
ASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

(19) United States
(12) Patent Application Publication
(10) Pub. No.: US 2026/0097134 A1
(43) Pub. Date: Apr. 9, 2026

(54) COMPOSITIONS AND METHODS FOR PRECISE EDITING OF HUMAN DYSTROPHIN

(71) Applicant: Mammoth Biosciences, Inc., Brisbane, CA (US)

(72) Inventors: Yuchen GAO, South San Francisco, CA (US); Ning CHAI, Dublin, CA (US); Wiputra Jaya HARTONO, San Francisco, CA (US); Benjamin Julius RAUCH, San Francisco, CA (US); Aaron DELOUGHERY, San Francisco, CA (US); Stepan TYMOSHENKO, Sacramento, CA (US); William Douglass WRIGHT, Fairfield, CA (US); Paula GONCALVES CERQUEIRA, Benicia, CA (US); Brian R. CHAIKIND, Orinda, CA (US); Kevin Cristopher WILKINS, San Francisco, CA (US)

(73) Assignee: Mammoth Biosciences, Inc., Brisbane, CA (US)

(21) Appl. No.: 19/410,151

(22) Filed: Dec. 5, 2025

Related U.S. Application Data
(63) Continuation of application No. PCT/US2024/032491, filed on Jun. 5, 2024.
(60) Provisional application No. 63/506,803, filed on Jun. 7, 2023.
(60) Provisional application No. 63/514,583, filed on Jul. 20, 2023.
(60) Provisional application No. 63/584,230, filed on Sep. 21, 2023.
(60) Provisional application No. 63/636,160, filed on Apr. 19, 2024.
(60) Provisional application No. 63/649,215, filed on May 17, 2024.

(51) Int. Cl. A61K 48/00; C12N 9/12; C12N 9/22; C12N 15/11; C12N 15/86; C12N 15/90

(52) U.S. Cl. CPC A61K 48/0058; C12N 9/1276; C12N 9/224; C12N 15/11; C12N 15/86; C12N 15/907; C12Y 207/07049; C12N 2310/20; C12N 2750/14143

(57) ABSTRACT

Disclosed herein are systems, compositions, and methods for modifying the human dystrophin gene (DMD). Systems, compositions, and methods may comprise a compact Type V CRISPR-associated (Cas) protein, an RNA-dependent DNA polymerase, and/or one or more guide nucleic acids or uses thereof. These systems, compositions, and methods may be useful for treating diseases such as Duchenne muscular dystrophy (DMD).

### CROSS-REFERENCE TO RELATED APPLICATIONS

The present application is a continuation of Internation PCT Application No. PCT/US2024/032491, filed Jun. 5, 2024, which claims priority to U.S. Provisional Application 63/506,803, filed Jun. 7, 2023; U.S. Provisional Application 63/514,583, filed Jul. 20, 2023; U.S. Provisional Application 63/584,230, filed Sep. 21, 2023; U.S. Provisional Application 63/636,160, filed Apr. 19, 2024; and U.S. Provisional Application 63/649,215, filed May 17, 2024, the contents each of which are incorporated herein by reference in their entireties.

### REFERENCE TO THE ELECTRONIC SEQUENCE FILE

The contents of the electronic sequence listing (MABI_039_06US_SeqList_ST26.xml; Size: 1,338,629 bytes; and Date of Creation: Sep. 10, 2025) are herein incorporated by reference in its entirety.

### BACKGROUND

Duchenne Muscular Dystrophy (DMD) is a severe X-linked recessive neuromuscular disorder effecting approximately 1 in 4,000 live male births. It is caused by mutations in the dystrophin gene (DMD) (Chromosome X: 31,117,228-33,344,609 (Genome Reference Consortium-GRCh38/hg38)). With a genomic region of over 2.2 megabases in length, D) MI) is the second largest human gene. The dystrophin gene contains 79 exons that are processed into an mRNA having a length of about 11 kb that is translated into a 427 kDa dystrophin protein. The dystrophin protein forms a component of the dystrophin-glycoprotein complex (DGC), which bridges the inner cytoskeleton and the extracellular matrix.

Functionally, dystrophin acts as a linker between the actin filaments and the extracellular matrix within muscle fibers. The N-terminus of dystrophin is an actin binding domain, while the C-terminus interacts with a transmembrane scaffold that anchors the muscle fiber to the extracellular matrix. Upon muscle contraction, dystrophin provides structural support that allows the muscle tissue to withstand mechanical force. DMD may be caused by a wide variety of mutations within the dystrophin gene that result in premature stop codons and therefore a truncated dystrophin protein. Truncated dystrophin proteins do not contain the C-terminus of wildtype dystrophin, and therefore cannot provide the structural support necessary to withstand the stress of muscle contraction. As a result, the muscle fibers pull themselves apart, which leads to muscle wasting.

Patients are generally diagnosed by the age of 4, and wheelchair bound by the age of 10. Most patients do not live past the age of 25 due to cardiac and/or respiratory failure. Existing treatments are palliative at best. The most common treatment for DMD is steroids, which are used to slow the loss of muscle strength. However, because most DMD patients start receiving steroids early in life, the treatment delays puberty and further contributes to the patient's diminished quality of life. Thus, there remains a need for compositions, systems and methods for treating disorders associated with the dystrophin gene, such as DMD.

### SUMMARY

The present disclosure provides compositions and methods for precision editing of target nucleic acids of the dystrophin gene (D) MI)). In some embodiments, the present disclosure provides systems and compositions that comprise an RNA-dependent DNA polymerase (RDDP), an effector protein (e.g., a CRISPR associated (Cas) protein), a guide RNA, and a template RNA. In some embodiments, the guide RNA and template RNA (retRNA) are linked as an extended guide RNA (e.g., rtgRNA). In some embodiments, the guide RNA and retRNA are not linked. In some embodiments, the present disclosure further provides methods of modifying DMD utilizing the systems and compositions described herein.

In some embodiments, the present disclosure provides fusion proteins, systems, and methods for precision editing. In general, precision editing is an approach for gene editing using an effector protein (e.g., a Cas protein), a polymerase (e.g., an RDDP), and a template RNA with a desired edit to introduce specific gene edits into a target sequence of D) MI). In many instances, RDDPs and effector proteins provided herein comprise less than 500 amino acids, thereby enabling the combination of their coding sequences into a single delivery vector (either as two separate proteins or as a fusion protein). Furthermore, in some embodiments, the RDDPs provided herein demonstrate enhanced editing efficiencies compared to previously described enzymes and can therefore be used in various embodiments to enhance editing of target genes.

Compositions, systems, and methods disclosed herein may leverage nucleic acid modifying activities. Nucleic acid modifying activities may include cis cleavage activity, nicking activity, or nucleobase modifying activity. Compositions, systems and methods disclosed herein may be useful for modifying a single nucleotide of a target nucleic acid. Accordingly, compositions, systems and methods disclosed herein may be useful for correcting a sequence comprising a mutation in a wildtype sequence. Such gene editing may be referred to as “precision editing.” In some instances, compositions, systems, and methods are useful for the treatment of a disease or disorder. The disease or disorder may be associated with one or more mutations in the target nucleic acid of DMD.

In some aspects, the present disclosure provides systems comprising: (a) an effector protein or a nucleic acid encoding the effector protein; (b) an RNA-directed DNA polymerase (RDDP) or a nucleic acid encoding the RDDP; (c) a guide RNA or a nucleic acid encoding the guide RNA, wherein the guide RNA comprises (i) a first region comprising a protein binding sequence, and (ii) a second region comprising a spacer sequence that hybridizes to a target sequence of a first strand of a double stranded DNA (dsDNA) target nucleic acid, wherein the first region is located 5′ of the second region; and (d) a template RNA (retRNA) or a nucleic acid encoding the retRNA, wherein the retRNA comprises (i) a primer binding sequence (PBS), and (ii) a template sequence that hybridizes to the target sequence of a second strand of the dsDNA target nucleic acid. In some embodiments, the guide RNA and the retRNA are not linked. In some embodiments, the template sequence is located 5′ of the PBS, optionally wherein the 3′ end of the PBS is linked to the 5′ end of the protein binding sequence. In some embodiments, the retRNA is circularized. In some embodiments, the retRNA comprises a protein localization sequence that can localize a protein to the retRNA. In some embodiments, the protein localization sequence comprises an MS2 coat protein localization sequence. In some embodiments, the RDDP is not linked to the effector protein, optionally wherein the RDDP is fused to an MS2 coat protein. In some embodiments, the RDDP is linked to the effector protein. In some embodiments, the template sequence comprises a difference of at least one nucleotide relative to an equal length portion of the target sequence. In some embodiments, the effector protein is a Type V Cas protein. In some embodiments, the length of the effector protein is 400 to 800 linked amino acids. In some embodiments, the effector protein comprises an amino acid sequence that is at least 65%, at least 70%, at least 75%, at least 80%, at least 85%, at least 90%, at least 95%, at least 97%, at least 98%, at least 99%, or 100% identical to a sequence in TABLE 1. In some embodiments, the effector protein comprises at least one amino acid alteration relative to a relative amino acid sequence in TABLE 1 that results in reduced nuclease activity, increased nickase activity, or a combination thereof. In some embodiments, the effector protein is a nickase, or wherein the effector protein has nicking activity. In some embodiments, the target nucleic acid comprises a PAM sequence and at least a portion of the PBS is complementary to at least a portion of the target nucleic acid sequence that is 5′ of the nucleotide at position 13 relative to the PAM sequence. In some embodiments, the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 1. In some embodiments, the effector protein comprises at least one amino acid alteration relative to the amino acid sequence of SEQ ID NO: 1. In some embodiments, the at least one amino acid alteration comprises an amino acid alteration set forth in TABLE 1.1. In some embodiments, the at least one amino acid alteration is selected from D220R and A306K, and D220R and K250N. In some embodiments, the guide RNA comprises a nucleotide sequence that is at least 90%, at least 95%, at least 97%, at least 98%, at least 99%, or 100% identical to any one of the nucleotide sequences of SEQ ID NOs: 466-648. In some embodiments, the guide RNA comprises the spacer sequence that hybridizes to a target sequence of DMD and comprises a nucleotide sequence that is at least 90%, at least 95%, at least 97%, at least 98%, at least 99%, or 100% identical to any one of the nucleotide sequences of SEQ ID NOs: 100-282. In some embodiments, the guide RNA comprises the handle sequence comprising a nucleotide sequence that is at least 90%, at least 95%, at least 97%, at least 98%, at least 99%, or 100% identical to the nucleotide sequences of SEQ ID NO: 283. In some embodiments, the guide RNA comprises a nucleotide sequence that is at least 90%, at least 95%, at least 97%, at least 98%, at least 99%, or 100% identical to any one of the nucleotide sequences of SEQ ID NOs: 649-831. In some embodiments, the retRNA comprises a nucleotide sequence that is at least 90%, at least 95%, at least 97%, at least 98%, at least 99%, or 100% identical to any one of the nucleotide sequences of SEQ ID NOs: 880-903. In some embodiments, the retRNA comprises the PBS comprising a nucleotide sequence that is at least 90%, at least 95%, at least 97%, at least 98%, at least 99%, or 100% identical to any one of the nucleotide sequences of SEQ ID NOs: 832-855. In some embodiments, the retRNA comprises the template sequence (RTT) comprising a nucleotide sequence that is at least 90%, at least 95%, at least 97%, at least 98%, at least 99%, or 100% identical to any one of the nucleotide sequences of SEQ ID NOs: 856-879. In some embodiments, the retRNA comprises a nucleotide sequence that is at least 90%, at least 95%, at least 97%, at least 98%, at least 99%, or 100% identical to any one of the nucleotide sequences of SEQ ID NOs: 904-927. In some embodiments, the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 2. In some embodiments, the effector protein comprises at least one amino acid alteration relative to the amino acid sequence of SEQ ID NO: 2. In some embodiments, the at least one amino acid alteration comprises an alteration set forth in TABLE 1.2. In some embodiments, the at least one amino acid alteration is selected from N355R, N148R, H208R, and a combination thereof. In some embodiments, the at least one amino acid alteration is selected from L26X and A121Q, K99R and L149R, L26X and N148R, L26X and H208R, N30R and N148R, L26X and N355R, L26X and K99R, L26X and K348R, L26X and A121Q, K99R and N148R, L149R and H208R, L26X and L149R, and S362R and L26X, and any combination thereof, wherein X is selected from R or K. In some embodiments, the guide RNA comprises a nucleotide sequence that is at least 90%, at least 95%, at least 97%, at least 98%, at least 99%, or 100% identical to any one of the nucleotide sequences of SEQ ID NOs: 1212-1353. In some embodiments, the guide RNA comprises the spacer sequence that hybridizes to a target sequence of DMD and comprises a nucleotide sequence that is at least 90%, at least 95%, at least 97%, at least 98%, at least 99%, or 100% identical to any one of the nucleotide sequences of SEQ ID NOS: 928-1069. In some embodiments, the guide RNA comprises the handle sequence comprising a nucleotide sequence that is at least 90%, at least 95%, at least 97%, at least 98%, at least 99%, or 100% identical to the nucleotide sequence of SEQ ID NO: 6. In some embodiments, the guide RNA comprises a nucleotide sequence that is at least 90%, at least 95%, at least 97%, at least 98%, at least 99%, or 100% identical to any one of the nucleotide sequences of SEQ ID NOs: 1354-1495. In some embodiments, the retRNA comprises a nucleotide sequence that is at least 90%, at least 95%, at least 97%, at least 98%, at least 99%, or 100% identical to any one of the nucleotide sequences of SEQ ID NOs: 1544-1567. In some embodiments, the retRNA comprises the PBS comprising a nucleotide sequence that is at least 90%, at least 95%, at least 97%, at least 98%, at least 99%, or 100% identical to any one of the nucleotide sequences of SEQ ID NOs: 1496-1519. In some embodiments, the retRNA comprises the RTT comprising a nucleotide sequence that is at least 90%, at least 95%, at least 97%, at least 98%, at least 99%, or 100% identical to any one of the nucleotide sequences of SEQ ID NOs: 1520-1543. In some embodiments, the retRNA comprises a nucleotide sequence that is at least 90%, at least 95%, at least 97%, at least 98%, at least 99%, or 100% identical to any one of the nucleotide sequences of SEQ ID NOs: 1568-1591. In some embodiments, the primer binding sequence is less than 20, less than 19, less than 18, less than 17, less than 16, less than 16, less than 15, less than 14, less than 13, less than 12, less than 11, less than 10, less than 9, less than 8, less than 7, less than 6, less than 5 nucleotides long, and at least 4 nucleotides long. In some embodiments, the template sequence is less than 35, less than 34, less than 33, less than 32, less than 31, less than 30, less than 29, less than 28, less than 27, less than 26, less than 25, less than 24, less than 23, less than 22, less than 21, less than 20, less than 19, less than 18 nucleotides long, and at least 8 nucleotides long. In some embodiments, the RDDP comprises an amino acid sequence that is at least 65%, at least 70%, at least 75%, at least 80%, at least 85%, at least 90%, at least 95%, at least 97%, at least 98%, at least 99%, or 100% identical to any one of SEQ ID NOs: 11-89. In some embodiments, the RDDP comprises an amino acid sequence that is at least 65%, at least 70%, at least 75%, at least 80%, at least 85%, at least 90%, at least 95%, at least 97%, at least 98%, at least 99%, or 100% identical to any one of SEQ ID NOs: 22, 24, 30, 32, or 40. In some embodiments, the effector protein comprises four amino acid substitutions relative to SEQ ID NO: 2, wherein the amino acid substitutions comprise L26R. 1471T. S223P, and D703G. In some embodiments, the effector protein comprises an amino acid sequence that is at least 90%, at least 95%, at least 97%, at least 98%, at least 99%, or 100% identical to the amino acid sequence of SEQ ID NO: 1607. In some embodiments, the RDDP is not linked to the effector protein and the RDDP is linked to an MS2 coat protein (MCP). In some embodiments, the RDDP comprises an amino acid sequence that is at least 90%, at least 95%, at least 97%, at least 98%, at least 99%, or 100% identical to the amino acid sequence of SEQ ID NO: 1608. In some embodiments, the guide RNA comprise a nucleotide sequence that is at least 90%, at least 95%, at least 97%, at least 98%, at least 99%, or 100% identical to the nucleotide sequence of SEQ ID NO: 1327. In some embodiments, the protein binding sequence comprises a nucleotide sequence that is at least 90%, at least 95%, at least 97%, at least 98%, at least 99%, or 100% identical to the nucleotide sequence of SEQ ID NO: 6. In some embodiments, the spacer sequence comprises a nucleotide sequence that is at least 90%, at least 95%, at least 97%, at least 98%, at least 99%, or 100% identical to the nucleotide sequence of SEQ ID NO: 1043. In some embodiments, the retRNA comprises a nucleotide sequence that is at least 90%, at least 95%, at least 97%, at least 98%, at least 99%, or 100% identical to the nucleotide sequence of SEQ ID NO: 1590. In some embodiments, the PBS comprises a nucleotide sequence that is at least 90%, at least 95%, at least 97%, at least 98%, at least 99%, or 100% identical to the nucleotide sequence of SEQ ID NO: 1518. In some embodiments, the RTT comprises a nucleotide sequence that is at least 90%, at least 95%, at least 97%, at least 98%, at least 99%, or 100% identical to the nucleotide sequence of SEQ ID NO: 1542. In some embodiments, the system comprises an expression vector, wherein the expression vector comprises any combination of: the nucleic acid encoding the effector protein; the nucleic acid encoding the RDDP; the nucleic acid encoding the guide RNA; and the nucleic acid encoding the retRNA. In some embodiments, the expression vector is an adeno-associated viral (AAV) vector, optionally wherein the AAV vector is an scAAV vector. In some embodiments, the system comprises a lipid or lipid nanoparticle. In some embodiments, the nucleic acid encoding the effector protein or the nucleic acid encoding the RDDP comprises a messenger RNA. In some embodiments, the system comprises a non-homologous end joining (NHEJ) inhibitor.

In some aspects, the present disclosure provides expression cassettes comprising, from 5′ to 3′: a) a first inverted terminal repeat (ITR); b) a first promoter sequence operably linked to a nucleic acid sequence encoding a guide RNA wherein the guide RNA comprises: i) a first region comprising a protein binding sequence; and ii) a second region comprising a spacer sequence that is complementary to a target sequence of DMD), wherein the spacer sequence comprises a nucleic acid sequence selected from SEQ ID NOs: 100-282 and 928-1069; c) a second promoter sequence operably linked to a nucleic acid sequence encoding an effector protein; d) a poly(A) signal; and e) a second ITR. In some embodiments, the expression cassette further comprises a WPRE sequence located between the nucleic acid sequence encoding an effector protein and the poly(A) signal. In some embodiments, the first promoter is a U6 promoter. In some embodiments, the second promoter is a CK8E promoter or a SPC5 promoter. In some embodiments, the poly(A) signal is a bGH or an hGH poly(A) signal. In some embodiments, the effector protein comprises an amino acid sequence that is at least 90% identical to SEQ ID NO:2. In some embodiments, the effector protein comprises the amino acid substitution of L26R relative to SEQ ID NO: 2. In some embodiments, the effector protein comprises an amino acid sequence that is at least 90% identical to SEQ ID NO:1. In some embodiments, the effector protein comprises the amino acid substitution of D220R relative to SEQ ID NO: 1.

In some aspects, the present disclosure provides adeno-associated virus (AAV) vectors comprising an expression cassette described herein.

In some aspects, the present disclosure provides pharmaceutical compositions comprising any one of the effector protein, RDDP, guide RNA, template RNA, and a nucleic acid encoding the same; and a pharmaceutically acceptable excipient.

In some aspects, the present disclosure provides cells modified by a system described herein. In some embodiments, the present disclosure provides cells comprising a system described herein. In some embodiments, the cell is a eukaryotic cell.

In some aspects, the present disclosure provides methods of modifying a target nucleic acid in a cell, the method comprising contacting a target nucleic acid with a system described herein. In some embodiments, the cell is a eukaryotic cell. In some embodiments, the target nucleic acid is DMD.

In some aspects, provided herein are methods of treating a disease associated with a mutation of a human dystrophin gene in a subject in need thereof, the method comprising administering to the subject: (a) any one of the systems described herein; or (b) any one of the pharmaceutical compositions described herein; or (c) any one of the AAV vectors described herein. In some embodiments, the disease or disorder is any one of the diseases or disorders set forth in TABLE 13. In some embodiments, the disease or disorder is Duchenne muscular dystrophy (DMD), becker muscular dystrophy (BMD), or x-linked dilated cardiomyopathy (CMD) Type 3B. In some embodiments, the disease is DMD.

In some aspects, provided herein are systems for deleting a region of D) MI), the system comprising: (a) an effector protein or a nucleic acid encoding the effector protein, wherein the effector protein forms a dimer with itself in a cell; (b) an RNA-directed DNA polymerase (RDDP) or a nucleic acid encoding the RDDP: (c) a first guide RNA (gRNA) or a nucleic acid encoding the first gRNA, wherein the first gRNA comprises (i) a first scaffold sequence, and (ii) a first spacer sequence that hybridizes to a first target sequence on a first strand of the DMD gene, wherein the first scaffold sequence is located 5′ of the first spacer sequence, and wherein the effector protein and the first gRNA form a first RNP complex that cleaves the DMD gene to form a first single stranded DNA (ssDNA) flap from the first strand of the DMD gene; (d) a second gRNA or a nucleic acid encoding the second gRNA, wherein the second gRNA comprises (i) a second scaffold sequence, and (ii) a second spacer sequence that hybridizes to a second target sequence on a second strand of the DMD gene, wherein the second scaffold sequence is located 5′ of the second spacer sequence, and wherein the effector protein and the second gRNA form a second RNP complex that cleaves the DMD gene to form a second ssDNA flap from the second strand of the DMD gene; (e) a first template RNA (retRNA) or a nucleic acid encoding the first retRNA, wherein the first retRNA comprises (i) a first primer binding sequence (PBS) that hybridizes to at least a portion of the first ssDNA flap, and (ii) a first template sequence; and (f) a second retRNA or a nucleic acid encoding the second retRNA, wherein the second retRNA comprises (i) a second PBS that hybridizes to at least a portion of the second ssDNA flap, and (ii) a second template sequence. In some embodiments, the nucleic acid encoding the effector protein, the RDDP, the gRNAs, and the retRNAs are combined in a single AAV vector. In some embodiments, the first spacer sequence and the second spacer sequence hybridize to the first target sequence and the second target sequence of the DMD gene respectively, wherein the first target sequence and the second target sequence are 10 to 10,000 base pairs apart. In some embodiments, the system comprises a Brex27 peptide or a nucleic acid encoding the Brex27 peptide, optionally wherein the Brex27 peptide comprises an amino acid sequence that is at least 90%, at least 95%, at least 98%, at least 99%, or 100% identical to SEQ ID NO: 1625.

In some aspects, provided herein are systems for modifying a target strand (TS) of a human dystrophin gene (I) MI)) gene, the system comprising: (a) an effector protein or a nucleic acid encoding the effector protein, wherein the effector protein forms a dimer with itself in a cell; (b) an RNA-directed DNA polymerase (RDDP) or a nucleic acid encoding the RDDP; (c) a guide RNA (gRNA) or a nucleic acid encoding the guide RNA, wherein the guide RNA comprises (i) a first region comprising a scaffold sequence, and (ii) a second region comprising a spacer sequence that hybridizes to a target sequence of the TS of the DMD gene, wherein the first region is located 5′ of the second region; and wherein the effector protein and the gRNA form a RNP complex that produces a double stranded break; (d) a TS template RNA (retRNA) or a nucleic acid encoding the TS retRNA, wherein the TS retRNA comprises (i) a TS primer binding sequence (PBS) that hybridizes to the TS, and (ii) a TS template sequence. In some embodiments, the effector protein comprises an amino acid sequence that is at least 70%, at least 75%, at least 80%, at least 85%, at least 90%, at least 95%, at least 97%, at least 98%, at least 99%, or 100% identical to SEQ ID NO: 1. In some embodiments, the effector protein comprises at least one amino acid substitution relative to SEQ ID NO: 1, wherein the amino acid substitution is D220R. In some embodiments, the effector protein is linked to RDDP to form a fusion protein. In some embodiments, the fusion protein comprises an amino acid sequence that is at least 70%, at least 75%, at least 80%, at least 85%, at least 90%, at least 95%, at least 97%, at least 98%, at least 99%, or 100% identical to an amino acid sequence selected from SEQ ID NOs: 1610-1619. In some embodiments, the fusion protein comprises an amino acid sequence that is at least 70%, at least 75%, at least 80%, at least 85%, at least 90%, at least 95%, at least 97%, at least 98%, at least 99%, or 100% identical to an amino acid sequence selected from SEQ ID NOs: 1611, 1613, 1615, 1618, and 1619, wherein the amino acid sequence comprises a P2A peptide that results in cleavage of the fusion protein at the site of the P2A. In some embodiments, the gRNA comprises a nucleic acid sequence that is at least 70%, at least 75%, at least 80%, at least 85%, at least 90%, at least 95%, at least 97%, at least 98%, at least 99%, or 100% identical to SEQ ID NO: 1620 or 1621. In some embodiments, the retRNA comprises a nucleic acid sequence that is at least 70%, at least 75%, at least 80%, at least 85%, at least 90%, at least 95%, at least 97%, at least 98%, at least 99%, or 100% identical to a sequence of any one of SEQ ID NO: 1622-1624. In some embodiments, the nucleic acid sequences encoding the effector protein, the RDDP, the gRNA, and the TS retRNAs are combined in a single AAV vector. In some embodiments, a nucleic acid sequence encoding the effector protein and a nucleic acid sequence encoding the RDDP is linked by a nucleic acid sequence encoding a P2A peptide.

### DESCRIPTION OF FIGURES

FIG. 1A shows the orientation of an effector protein binding to the target strand (T-strand, TS) and the relative positions of the RuvC sites to the PAM sequence. FIG. 1B shows the position of the cleavage site on the nontarget strand (NT-strand, NTS) and/or the target strand for effector proteins relative to the PAM sequence.

FIG. 2 shows exemplary plasmids for compositions, systems and methods disclosed herein. In other embodiments, plasmids encode a guide RNA comprising a spacer sequence and a protein binding sequence wherein the guide RNA is separate from a primer binding sequence linked to a template sequence. In some embodiments, the plasmids encode an effector protein and a partner protein that are not linked, instead of a fusion protein.

FIG. 3 shows an exemplary gene editing system comprising an extended guide RNA with the primer binding sequence and template sequence located 5′ of a repeat sequence and a spacer sequence. The effector protein is linked to the RNA-directed DNA polymerase (RDDP). System components shown in FIG. 3 are exemplary and non-limiting. For instance, the 5′ extended rtgRNA may comprise additional nucleotides beyond those labeled as spacer, repeat, linker, PBS and RT template (RTT).

FIG. 4 shows an exemplary gene editing system comprising an extended guide RNA with the primer binding sequence and template sequence located 3′ of a repeat sequence and a spacer sequence. The effector protein is linked to the RNA-directed DNA polymerase (RDDP). System components shown in FIG. 4 are exemplary and non-limiting. For instance, the 5′ extended rtgRNA may comprise additional nucleotides beyond those labeled as spacer, repeat, linker, PBS and RT template (RTT).

FIGS. 5A-5B show exemplary split protein RNA design gene editing systems comprising a guide RNA with the repeat and spacer sequences separate from the retRNA that comprises the primer binding sequence and template sequence. The effector protein is separate from the RDDP. The effector protein is localized to the target nucleic acid via the guide RNA where it can nick the target nucleic acid. The retRNA may comprise a secondary structure. The secondary structure may comprise an aptamer, and the RDDP may comprise a peptide that is capable of binding the aptamer, thereby localizing the RDDP to the nicked DNA. System components shown in FIGS. 5A and 5B are exemplary and non-limiting. For instance, the gRNA and/or retRNA may comprise additional nucleotides beyond those labeled as spacer, repeat, linker, PBS and RT template (RTT).

FIG. 6 shows an overview of a system comprising CasM.265466 for precise editing. CasM.265466 generates a double-stranded break on target DNA. A retRNA hybridizes with the non-target strand (as shown) or target strand (not shown) via complementarity between the PBS region and the target DNA. An RDDP is recruited to the target site by binding of a linked MS2 coat protein to the MS2 aptamer of the retRNA. The RNA/DNA hybrid serves as a binding substrate for the RDDP, which then synthesizes new cDNA along the exposed 3′ end of the target DNA using the RTT as a reverse transcription template. Edits encoded in the RTT are thereby written into the genome.

FIG. 7A and FIG. 7B show how CasM.265466 is predicted to edit the non-target strand (NTS) and the target strand (TS) of a double stranded DNA (dsDNA) target nucleic acid. FIG. 7A shows NTS editing. FIG. 7B shows TS editing.

FIG. 8 shows an example of a split protein/RNA system for precise editing with Type II Cas effectors (e.g., Cas9), wherein the retRNA is circularized.

FIG. 9 shows an example of a split protein/RNA system for precise editing with Type V Cas effectors, wherein the retRNA is circularized. CasPhi (e.g., CasPhi.12 disclosed herein) is a non-limiting example of a Type V Cas effector.

FIG. 10 depicts in vivo gene editing of PCSK9 in muscle tissues using AAV9-A4 delivery of CasPhi. 12 and CasM.265466 variants.

FIG. 11 illustrates an exemplary dual-cut dual-flap system for precise deletion of a region of dsDNA.

FIG. 12 illustrates an exemplary precision editing system using a Type V Cas nuclease that creates double stranded breaks with the target strand being extended by the RT.

### DETAILED DESCRIPTION

It is to be understood that both the foregoing general description and the following detailed description are exemplary, and explanatory only, and are not restrictive of the disclosure.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### Definitions

Unless otherwise indicated, all technical terms used herein have the same meaning as commonly understood by one of ordinary skill in the art. Unless otherwise indicated or obvious from context, the following terms have the following meanings:

As used in this specification and the appended claims, the singular forms “a,” “an,” and “the” include plural references unless the context clearly dictates otherwise.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term “comprise” and its grammatical equivalents specifies the presence of stated features, integers, steps, operations, elements, and/or components, but does not preclude the presence or addition of one or more other features, integers, steps, operations, elements, components, and/or groups thereof.

As used herein, the term “about” in reference to a number or range of numbers is understood to mean the stated number and numbers +/−10% thereof, or 10% below the lower listed limit and 10% above the higher listed limit for the values listed for a range.

The terms “% identical,” “% identity,” and “percent identity,” or grammatical equivalents thereof, with reference to an amino acid sequence or nucleotide sequence, refer to the percent of residues that are identical between respective positions of two sequences when the two sequences are aligned for maximum sequence identity. The % identity is calculated by dividing the total number of the aligned residues by the number of the residues that are identical between the respective positions of the at least two sequences and multiplying by 100. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95). To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a CAS polypeptide/guide RNA complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (KD) of less than 10-6 M, less than 10-7 M, less than 10-8 M, less than 10-9 M, less than 10-10 M, less than 10-11 M, less than 10-12 M, less than 10-13 M, less than 10-14 M, or less than 10-15 M. “Affinity” refers to the strength of binding, increased binding affinity being correlated with a lower KD.

By “binding domain” it is meant a protein or nucleic acid domain that is able to bind non-covalently to another molecule. A binding domain can bind to, for example, a DNA molecule (a DNA-binding domain), an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity but can be nickase activity. The naturally-occurring effector protein may be a wild-type protein. In some instances, the catalytically inactive effector protein is referred to as a catalytically inactive variant of an effector protein, e.g., a Cas effector protein.

The term, “cis cleavage,” as used herein, refers to cleavage (hydrolysis of a phosphodiester bond) of a target nucleic acid by a complex of an effector protein and a guide nucleic acid, wherein at least a portion of the guide nucleic acid is hybridized to at least a portion of the target nucleic acid. Cleavage may occur within or directly adjacent to the portion of the target nucleic acid that is hybridized to the portion of the guide nucleic acid.

The terms, “cleave,” “cleaving,” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The terms, “complementary” and “complementarity,” as used herein, with reference to a nucleic acid molecule or nucleotide sequence, refer to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term, “conservative substitution” as used herein refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (G); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T). Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl. Amino acids may be related by aromatic side chains: Phe (F), Tyr (Y), Trp (W). Amino acids may be related by amide side chains: Asn (N), Glu (Q). Amino acids may be related by sulfur-containing side chains: Cys (C) and Met (M).

The term, “cleavage assay,” as used herein, refers to an assay designed to visualize, quantitate or identify cleavage of a nucleic acid. In some instances, the cleavage activity may be cis-cleavage activity.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from another organism.

The term, “donor nucleic acid,” as used herein, refers to a nucleic acid that is (designed or intended to be) incorporated into a target nucleic acid or target sequence.

The term, “% editing efficiency.” as used herein, refers to the percent of target nucleic acids in a sample or population of cells exhibiting an edited target nucleic acid. Editing efficiency may also be referred to as % editing level or % edited. There are multiple approaches to evaluate % editing efficiency, including, but not limited to, next generation sequence and real time PCR.

The term, “target nucleic acid,” as used herein, refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, when used in reference to a target nucleic acid, refers to a sequence of nucleotides found within a target nucleic acid. Such a sequence of nucleotides can, for example, hybridize to a respective length portion of a guide nucleic acid. Hybridization of the guide nucleic acid to the target sequence may bring an effector protein into contact with the target nucleic acid.

A nucleotide sequence that “encodes” a particular polypeptide or protein, is a nucleotide sequence that is transcribed into mRNA (in the case of DNA) and/or is translated (in the case of mRNA) into a polypeptide.

The term “transgene” as used herein refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgene expression occurs can be a target cell, such as a host cell.

The term, “functional fragment,” as used herein, refers to a fragment of a protein that retains some function relative to the entire protein. Non-limiting examples of functions are nucleic acid binding, protein binding, nuclease activity, nickase activity, deaminase activity, demethylase activity, or acetylation activity. A functional fragment may be a recognized functional domain, e.g., a catalytic domain such as, but not limited to, a RuvC domain.

The terms, “fusion effector protein,” “fusion protein,” and “fusion polypeptide,” may be used interchangeably herein and refer to a protein comprising at least two heterologous polypeptides. Often a fusion effector protein comprises an effector protein and a fusion partner protein. In general, the fusion partner protein is not an effector protein. Examples of fusion partner proteins are provided herein.

The terms “fusion partner protein” or “fusion partner.” as used herein, refer to a protein, polypeptide or peptide that is linked to an effector protein. The fusion partner generally imparts some function to the fusion protein that is not provided by the effector protein.

The term, “effector protein,” as used herein, refers to a polypeptide that non-covalently binds to a guide nucleic acid to form a complex that contacts a target nucleic acid, wherein at least a portion of the guide nucleic acid hybridizes to a target sequence of the target nucleic acid. A complex between an effector protein and a guide nucleic acid can include multiple effector proteins or a single effector protein. In some instances, the effector protein modifies the target nucleic acid when the complex contacts the target nucleic acid. In some instances, the effector protein does not modify the target nucleic acid, but it is linked to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout.

The term, “genetic disease,” as used herein, refers to a disease, disorder, condition, or syndrome associated with or caused by one or more mutations in the DNA of an organism having the genetic disease.

The term, “guide nucleic acid.” as used herein, refers to at least one nucleic acid comprising: a first nucleotide sequence that complexes to an effector protein on either the 5′ or 3′ terminus and the first nucleotide sequence can be linked to a second nucleotide sequence that hybridizes to a target nucleic acid. The first sequence may be referred to herein as a repeat sequence or guide sequence. The second sequence may be referred to herein as a spacer sequence. A guide nucleic acid may be referred to interchangeably with the term, “guide RNA.” It is understood that guide nucleic acids may comprise DNA, RNA, or a combination thereof (e.g., RNA with a thymine base). Guide nucleic acids may include a chemically modified nucleobase or phosphate backbone.

The term, “extended guide RNA (rtgRNA),” as used herein refers to a single nucleic acid molecule comprising (not necessarily in the following order) (1) a guide RNA comprising (a) a protein binding sequence and (b) a spacer sequence: (2) optionally, a linker; and (3) a template RNA (retRNA) comprising (a) a primer binding sequence and (b) a template sequence. In some embodiments, the orientation of the rtgRNA from 5′ to 3′ is: guide nucleic acid, optional linker, and template RNA. In some embodiments, the orientation of the rtgRNA from 5′ to 3′ is: template RNA, linker, and guide RNA. It is understood that extended guide RNAs may comprise DNA, RNA, or a combination thereof (e.g., RNA with a thymine base).

The term “template RNA (retRNA)” as used herein, refers to a nucleic acid comprising: a primer binding sequence and a template sequence. It is understood that template RNAs may comprise DNA, RNA, or a combination thereof (e.g., RNA with a thymine base). In some instances, the template RNA is linked to a guide RNA via a linker sequence to form an rtgRNA. Template RNA is used interchangeable with retRNA herein.

The term, “template sequence,” as used herein, refers to a portion of a retRNA that contains a desired nucleotide modification relative to a target sequence or portion thereof. By way of non-limiting example, the desired edit may comprise one or more nucleotide insertions, deletions or substitutions relative to a target sequence or portion thereof. In some embodiments, it is identical to, complementary to, or reverse complementary to a target sequence or portion thereof. In some embodiments, the template sequence is complementary to a sequence of the target nucleic acid that is adjacent to a nick site of a target site to be edited, with the exception that it includes a desired edit. The template sequence (also referred in some instances as the RT template (RTT)) can be complementary to at least a portion of the target sequence with the exception of at least one nucleotide.

The terms, “primer binding sequence (PBS),” as used herein, refer to a portion of a retRNA and serves to bind to a primer sequence of the target nucleic acid. In some embodiments, the primer binding sequence binds to a primer sequence in the target nucleic acid that is formed after the target nucleic acid is cleaved by an effector protein. In some embodiments, the primer binding sequence is linked to the 3′ end of a retRNA. In some embodiments, the primer binding sequence is located at the 5′ end of a retRNA.

“Primer sequence” as used herein refers to a portion of the target nucleic acid that is capable of hybridizing with the primer binding sequence portion of a retRNA that is generated after cleavage of the target nucleic acid by an effector protein described herein.

The term “handle sequence,” as used herein, refers to a sequence that binds non-covalently with an effector protein. A handle sequence may also be referred to herein as a “scaffold sequence”. In some instances, the handle sequence comprises all, or a portion of, a repeat sequence. In general, a single guide nucleic acid, also referred to as a single guide RNA (sgRNA), comprises a handle sequence that is capable of being non-covalently bound by an effector protein. The nucleotide sequence of a handle sequence may contain a portion of a tracrRNA, but generally does not comprise a sequence that hybridizes to a repeat sequence, also referred to as a repeat hybridization sequence.

The term “trans-activating RNA (tracrRNA),” as used herein, refers to a nucleic acid that comprises a first sequence that is capable of being non-covalently bound by an effector protein, and a second sequence that hybridizes to a portion of a crRNA, which may be referred to as a repeat hybridization sequence.

The terms, “CRISPR RNA” or “crRNA,” as used herein, refer to a type of guide nucleic acid, wherein the nucleic acid is RNA comprising a first sequence, often referred to herein as a spacer sequence, that hybridizes to a target sequence of a target nucleic acid, and a second sequence, often referred to herein as a repeat sequence or guide sequence, that interacts with an effector protein. In some instances, the second sequence is bound by the effector protein. In some instances, the second sequence hybridizes to a portion of a tracrRNA, wherein the tracrRNA forms a complex with the effector protein.

The term, “extension,” as used herein refers to additional nucleotides added to a nucleic acid, RNA, or DNA, or additional amino acids added to a peptide, polypeptide, or protein. Extensions may be processed during the formation of the guide RNA. In some instances, the extension comprises or consists of a template RNA.

By “hybridizable” or “complementary” or “substantially complementary” it is meant that a nucleic acid (e.g. RNA, DNA) comprises a sequence of nucleotides that enables it to noncovalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

The conditions of temperature and ionic strength determine the “stringency” of the hybridization. Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Temperature, wash solution salt concentration, and other conditions may be adjusted as necessary according to factors such as length of the region of complementation and the degree of complementation.

While hybridization typically occurs between two nucleotide sequences that are complementary, mismatches between bases are possible. It is understood that two nucleotide sequences need not be 100% complementary to be specifically hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

The conditions appropriate for hybridization between two nucleotide sequences depend on the length of the sequence and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Temperature, wash solution salt concentration, and other conditions may be adjusted as necessary according to factors such as length of the region of complementation and the degree of complementation. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

The term, “heterologous,” as used herein, with reference to at least two different polypeptide sequences, means that the two different polypeptide sequences are not found similarly connected to one another in a native nucleic acid or protein. A protein that is heterologous to the effector protein is a protein that is not covalently linked via an amide bond to the effector protein in nature. In some instances, a heterologous protein is not encoded by a species that encodes the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The term “linked” as used herein in reference to an amino acid or nucleic acid sequence refers to any covalent mechanism by which two amino acid sequences or nucleic acid sequences are connected to each other in sequence. For example, in some embodiments, two sequences are linked directly together by a covalent bond (e.g., an amide bond or phosphodiester bond). In some embodiments, two sequences are linked together by a peptide or nucleic acid linker. In some embodiments, two nucleotide sequences are linked by at least one nucleotide. In some embodiments, two amino acid sequences are linked by at least one amino acid.

The term, “linked amino acids” as used herein, refers to at least two amino acids linked by an amide bond or a peptide bond.

The term, “linker,” as used herein, refers to an amino acid sequence or nucleic acid sequence that links a first polypeptide to a second polypeptide or a first nucleic acid to a second nucleic acid.

The term, “modified target nucleic acid,” as used herein, refers to a target nucleic acid, wherein the target nucleic acid has undergone a modification, for example, after contact with an effector protein. In some instances, the modification is an alteration in the sequence of the target nucleic acid. In some instances, the modified target nucleic acid comprises an insertion, deletion, or replacement of one or more nucleotides compared to the unmodified target nucleic acid.

The terms “peptide,” “polypeptide,” and “protein” are used interchangeably herein, refer to a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more sequence modifications, or both. A peptide generally has a length of 100 or fewer linked amino acids.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways.

A DNA sequence that “encodes” a particular RNA is a DNA nucleotide sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

A “protein coding sequence” or a sequence that encodes a particular protein or polypeptide, is a nucleotide sequence that is transcribed into mRNA (in the case of DNA) and is translated (in the case of mRNA) into a polypeptide in vitro or in vivo when placed under the control of appropriate regulatory sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., RNA-guided endonuclease and the like) and/or regulate translation of an encoded polypeptide.

The term, “promoter” or “promoter sequence,” is a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The term, “protospacer adjacent motif (PAM),” as used herein, refers to a nucleotide sequence found in a target nucleic acid that directs an effector protein to modify the target nucleic acid at a specific location. In some instances, a PAM is required for a complex of an effector protein and a guide nucleic acid to hybridize to and modify the target nucleic acid. In some instances, the complex does not require a PAM to modify the target nucleic acid. One example of a PAM sequences is NTTN, where N can be any nucleic acid.

The term “RNA-dependent DNA polymerase (RDDP),” as used herein, refers to a DNA polymerase that uses a single-stranded RNA as a template for the synthesis of a complementary DNA strand.

The term, “RuvC” domain as used herein refers to a region of an effector protein that is capable of cleaving a target nucleic acid, and in certain instances, of processing a pre-crRNA. In some instances, the RuvC domain is located near the C-terminus of the effector protein. A single RuvC domain may comprise RuvC subdomains, for example a RuvCI subdomain, a RuvCII subdomain and a RuvCIII subdomain. The term “RuvC” domain can also refer to a “RuvC-like” domain. Various RuvC-like domains are known in the art and are easily identified using online tools such as InterPro (https://www.ebi.ac.uk/interpro/). For example, a RuvC-like domain may be a domain which shares homology with a region of TnpB proteins of the IS605 and other related families of transposons.

The term, “nickase” as used herein refers to an enzyme that possess catalytic activity for single stranded nucleic acid cleavage of a double stranded nucleic acid. A nickase cleaves a phosphodiester bond between two nucleotides of only one strand of dsDNA.

The terms, “nuclease” and “endonuclease” are used interchangeably herein to mean an enzyme which possesses catalytic activity for nucleic acid cleavage.

The term, “nuclease activity,” is used to refer to catalytic activity that results in nucleic acid cleavage (e.g., ribonuclease activity (ribonucleic acid cleavage), or deoxyribonuclease activity (deoxyribonucleic acid cleavage), etc.).

The term “naturally-occurring,” “unmodified.” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism that can be isolated from a source in nature is naturally occurring.

The terms, “non-naturally occurring” and “engineered.” as used herein, are used interchangeably and indicate the involvement of the hand of man. The terms, when referring to a nucleic acid, nucleotide, protein, polypeptide, peptide or amino acid, refer to a molecule, such as but not limited to, a nucleic acid, nucleotide, protein, polypeptide, peptide or amino acid refers to a modification of that molecule (e.g., chemical modification, nucleotide sequence, or amino acid sequence) that is not present in the naturally molecule. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term, “variant,” is intended to mean a form or version of a protein that differs from the wild-type protein. A variant may have a different function or activity relative to the wild-type protein.

The term, “sequence modification,” as used herein refers to a modification of one or more nucleic acid residues of a nucleotide sequence or one or more amino acid residue of an amino acid sequence, such as chemical modification of one or more nucleobases; or chemical modifications to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that, when transcribed, produces a guide nucleic acid). Methods of modifying a nucleic acid or amino acid sequence are known. One of ordinary skill in the art will appreciate that the sequence modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

The term, “nucleic acid expression vector,” as used herein, refers to a plasmid that can be used to express a nucleic acid of interest.

The term, “nuclear localization signal (NLS),” as used herein, refers to an entity (e.g., peptide) that facilitates localization of a nucleic acid, protein, or small molecule to the nucleus, when present in a cell that contains a nuclear compartment.

A person of ordinary skill in the art would appreciate that referring to a “nucleotide(s)”, and/or “nucleoside(s)”, in the context of a nucleic acid molecule having multiple residues, is interchangeable and describe the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a “nucleobase(s)”, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU).

Thus, the term “recombinant” polypeptide does not necessarily refer to a polypeptide whose amino acid sequence does not naturally occur. Instead, a “recombinant” polypeptide is encoded by a recombinant non-naturally occurring DNA sequence, but the amino acid sequence of the polypeptide can be naturally occurring (“wild type”) or non-naturally occurring (e.g., a variant, a mutant, etc.). A recombinant polypeptide is the product of a process run by a human or machine.

A “vector” or “expression vector” is a replicon, such as plasmid, phage, virus, artificial chromosome, or cosmid, to which another DNA segment, i.e., an “insert”, may be attached so as to bring about the replication of the attached segment in a cell.

The term “viral vector,” as used herein, refers to a nucleic acid to be delivered into a host cell via a recombinantly produced virus or viral particle.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence (or the coding sequence can also be said to be operably linked to the promoter) if the promoter affects its transcription or expression.

The terms “recombinant expression vector,” or “DNA construct” are used interchangeably herein to refer to a DNA molecule comprising a vector and an insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

A cell has been “genetically modified,” “transformed,” or “transfected” by exogenous DNA or exogenous RNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X (12) 00283-9. doi: 10.1016/j.addr.2012.09.023), and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

By “cleavage domain,” “active domain,” or “nuclease domain” of a nuclease it is meant the polypeptide sequence or domain within the nuclease which possesses the catalytic activity for nucleic acid cleavage. A cleavage domain can be contained in a single polypeptide chain or cleavage activity can result from the association of two (or more) polypeptides. A single nuclease domain may consist of more than one isolated stretch of amino acids within a given polypeptide.

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282(5391): 1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be target cells of the methods described herein.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26al, TERI, and zfp42. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g., Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

The terms, “treatment” or “treating,” as used herein, are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

A “syndrome”, as used herein, refers to a group of symptoms which, taken together, characterize a condition.

The terms “individual,” “subject,” “host,” and “patient,” used interchangeably herein, refer to an individual organism, e.g., a mammal, including, but not limited to, murines, simians, humans, non-human primates, ungulates, felines, canines, bovines, ovines, mammalian farm animals, mammalian sport animals, and mammalian pets.

The term, “subject,” as used herein, refers to an animal. The subject may be a mammal. The subject may be a human. The subject may be diagnosed or at risk for a disease.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

### INTRODUCTION

In some embodiments, the present disclosure provides systems and methods comprising: a) at least one of a polypeptide and/or a nucleic acid encoding the polypeptide; and b) at least one of a guide nucleic acid and/or a DNA molecule encoding the guide nucleic acid, and uses thereof, wherein the polypeptide and the guide nucleic acid form a complex that binds a target nucleic acid.

Polypeptides described herein may bind and, optionally, cleave nucleic acids in a sequence-specific manner. Polypeptides described herein may bind a target region of a target nucleic acid and cleave the target nucleic acid within the target region or at a position adjacent to the target region. In some embodiments, a polypeptide is activated when it binds a target region of a target nucleic acid to cleave a region of the target nucleic acid that is near, but not adjacent to the target region. A polypeptide may be an effector protein, such as a CRISPR-associated (Cas) protein, which may be coupled to a guide nucleic acid that imparts activity or sequence selectivity to the polypeptide. In general, guide nucleic acids comprise a first sequence that is at least partially complementary to a target nucleic acid, which may be referred to as a spacer sequence. In some embodiments, compositions, systems, and methods comprising effector proteins and guide nucleic acids can further comprise a second sequence, at least a portion of which interacts with the polypeptide. In some instances, the second sequence comprises a sequence that is similar or identical to a portion of a tracrRNA sequence, a CRISPR repeat sequence, or a combination thereof. In some embodiments, the guide nucleic acid does not comprise a tracrRNA.

Effector proteins disclosed herein may cleave nucleic acids, including single stranded RNA (ssRNA), double stranded DNA (dsDNA), and single-stranded DNA (ssDNA). Polypeptides disclosed herein may provide cis cleavage activity, trans cleavage activity, nickase activity, or a combination thereof. Cis cleavage activity is cleavage of a target nucleic acid that is hybridized to a guide nucleic acid (crRNA or sgRNA), wherein cleavage occurs within or directly adjacent to the region of the target nucleic acid that is hybridized to the guide nucleic acid. Nickase activity is the selective cleavage of one strand of a dsDNA molecule.

The compositions, systems and methods described herein are non-naturally occurring. In some instances, compositions, systems and methods comprise a guide nucleic acid or a use thereof. In some instances, compositions, systems and methods comprise an engineered polypeptide or a use thereof. In general, compositions and systems described herein are not found in nature. In some embodiments, compositions, methods and systems described herein comprise at least one non-naturally occurring component. For example, disclosed compositions, methods and systems may comprise a guide nucleic acid, wherein the sequence of the guide nucleic acid is different or modified from that of a naturally-occurring guide nucleic acid. In some embodiments, compositions, systems and methods comprise at least two components that do not naturally occur together. For example, disclosed compositions, methods and systems may comprise a guide nucleic acid comprising a repeat region and a spacer region which do not naturally occur together. Also, by way of non-limiting example, disclosed compositions, methods and systems may comprise a guide nucleic acid and an effector protein that do not naturally occur together. Conversely, and for clarity, an effector protein or guide nucleic acid that is “natural,” “naturally-occurring.” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural nucleotide sequence is a nucleotide sequence that is not found in nature. The non-natural nucleotide sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. Engineered guide nucleic acids may comprise a first sequence and a second sequence that do not occur naturally together. For example, a guide nucleic acid may comprise a sequence of a naturally-occurring repeat region and a spacer region that is complementary to a naturally-occurring eukaryotic sequence. The guide nucleic acid may comprise a sequence of a repeat region that occurs naturally in an organism and a spacer region that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. In some embodiments, the guide nucleic acid comprises two heterologous sequences arranged in an order or proximity that is not observed in nature. Therefore, compositions and systems described herein are not naturally occurring.

### Precision Editing Systems

In some embodiments, the present disclosure provides compositions, systems, and methods for precision editing. The present disclosure provides each of the components for such precision editing systems (e.g., effector proteins, RDDPs, and rtgRNAs) as well as systems comprising the individual components. In some embodiments, the effector protein and the RDDP are provided in the form of a fusion protein. In some embodiments, the effector protein and the RDDP are provided as two separate proteins. In some embodiments, the effector protein (or fusion protein comprising an effector protein) forms a complex with an extended guide RNA (rtgRNA).

In some embodiments, the effector protein is linked to an RDDP. In some embodiments, the RDDP comprises a reverse transcriptase. The effector protein may nick a strand of the target nucleic acid and the RDDP synthesizes new DNA off the nicked end, wherein the new DNA is complementary to the template sequence (RT template), thereby producing the desired genomic edit in the target nucleic acid. In some embodiments, the effector protein nicks the target strand. In some embodiments, the effector protein nicks the non-target strand.

In some embodiments, the effector protein nicks both the target strand and the non-target strand sequentially. For example, in some embodiments, the effector protein nicks the target strand first and facilitates, with an rtgRNA, the introduction of the desired genomic edit. After editing, the effector protein, in combination with a guide RNA that targets the non-target strand, can nick the non-target strand. The cellular DNA repair mechanisms can then repair the non-target strand using the edited target strand as template. See Anzalone, Nature. 2019 December; 576(7785):149-157.

In some embodiments, the systems provided herein comprise (a) a fusion protein described herein (or a polynucleotide encoding the same); and (b) an rtgRNA comprising a guide RNA and a template RNA. In some embodiments, the systems provided herein comprise an RDDP comprising (a) an amino acid sequence that is at least 90% or at least 95% identical to any one of SEQ ID NOs: 11-79; (b) an effector protein; and (c) an rtgRNA comprising a guide RNA and a template RNA.

In some embodiments, the systems provided herein comprise (a) a fusion protein described herein (or a polynucleotide encoding the same); (b) an rtgRNA comprising a guide RNA and a template RNA; and (c) a guide RNA. In some embodiments, the systems provided herein comprise an RDDP comprising (a) an amino acid sequence that is at least 90% or at least 95% identical to any one of SEQ ID NOs: 11-79; (b) an effector protein; (c) an rtgRNA comprising a guide RNA and a template RNA; and (d) a guide RNA.

In some embodiments, compositions, systems, and methods comprise a fusion protein or uses thereof. In general, a fusion protein comprises an effector protein that is covalently linked to a partner protein that is heterologous to the effector protein. In some embodiments, a partner protein comprises an enzyme that is capable of catalyzing the modification (insertion, deletion, or base-to-base conversion) of a target nucleic acid. In some instances, the partner protein is a polymerase. In some embodiments, the partner protein is an RNA-directed DNA polymerase (RDDP). In some embodiments, the RDDP is a reverse transcriptase. In some embodiments, compositions, systems, and methods disclosed herein, comprise an effector protein and a partner protein, or uses thereof, wherein the effector protein and the partner protein are not covalently linked.

In some embodiments, the enzyme that is capable of catalyzing the modification of the target nucleic acid forms a complex with an extended guide RNA (rtgRNA). In some embodiments, the extended guide RNA comprises (not necessarily in this order): a first region (also referred to as a protein binding region or protein binding sequence) that interacts with an effector protein; a second region comprising a spacer sequence that is complementary to a target sequence of a first strand of a target dsDNA molecule; a third region comprising a template sequence that is complementary to at least a portion of the target sequence on the non-target strand of the target dsDNA molecule with the exception of at least one nucleotide; and a fourth region comprising a primer binding sequence that hybridizes to a primer sequence of the target dsDNA molecule that is formed when target nucleic acid is cleaved. The third region or template sequence may comprise a nucleotide having a different nucleobase than that of a nucleotide at the corresponding position in the target nucleic acid when the template sequence and the target sequence are aligned for maximum identity. In some embodiments, there is a linker between any one of the first, second, third and fourth regions. In some embodiments, the linker comprises a nucleotide. In some embodiments, the linker comprises multiple nucleotides.

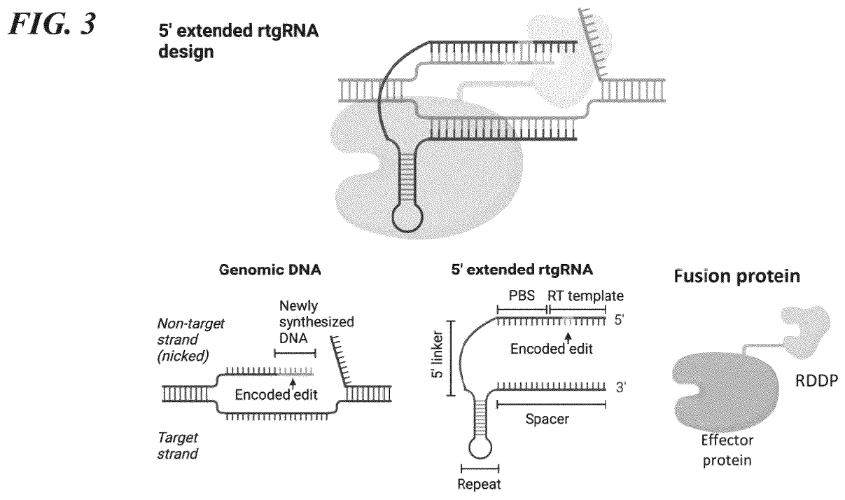

In some embodiments, the third and fourth regions are 5′ of the first and second regions. In some embodiments, the order of the regions of the extended guide RNA from 5′ to 3′ is: third region, fourth region, first region, and second region. In some embodiments, there is a linker between any one of the first, second, third and fourth regions. In some embodiments, there is a linker between the first and fourth regions. In some embodiments, the effector protein is linked to an RDDP. In some embodiments, the RDDP comprises a reverse transcriptase. See, e.g., FIG. 3. The effector protein may nick a strand of the target nucleic acid and the RDDP synthesizes new DNA off the nicked end, wherein the new DNA is complementary to the template sequence (RT template), thereby producing the desired genomic edit in the target nucleic acid. In some embodiments, the effector protein nicks the target strand. In some embodiments, the effector protein nicks the non-target strand.

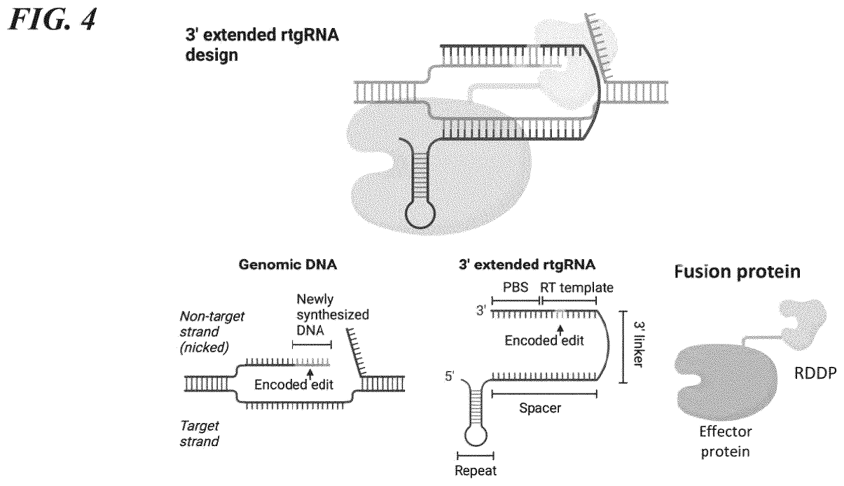

In some embodiments, the third and fourth regions are 3′ of the first and second regions. In some embodiments, the order of the regions of the extended guide RNA from 5′ to 3′ is: first region, second region, third region, and fourth region. In some embodiments, there is a linker between the second and third regions. In some embodiments, the effector protein is linked to an RDDP. In some embodiments, the RDDP comprises a reverse transcriptase. See, e.g., FIG. 4. The effector protein may nick a strand of the target nucleic acid and the RDDP synthesizes new DNA off the nicked end, wherein the new DNA is complementary to the template sequence (RT template), thereby producing the desired genomic edit in the target nucleic acid. In some embodiments, the effector protein nicks the target strand. In some embodiments, the effector protein nicks the non-target strand.

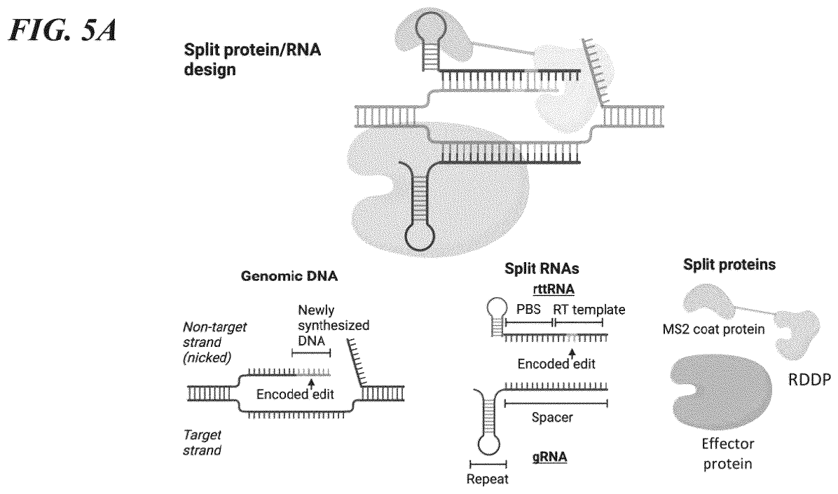

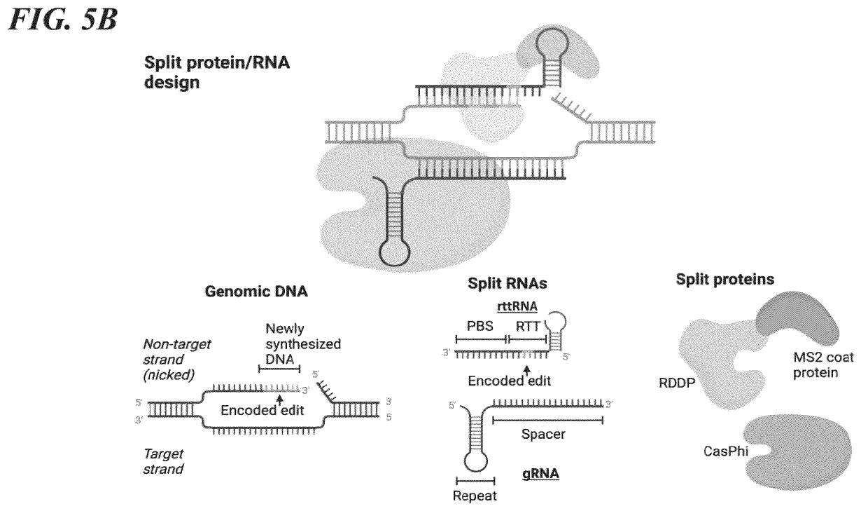

In some embodiments, compositions and systems comprise (1) a guide RNA comprising (a) a first region (also referred to as a protein binding region or protein binding sequence) that interacts with an effector protein and (b) a second region comprising a spacer sequence that is complementary to a target sequence of a first strand of a target dsDNA molecule; and (2) a template RNA (retRNA) comprising (a) a primer binding sequence that hybridizes to a primer sequence of the target dsDNA molecule that is formed when the target nucleic acid is cleaved and (b) a template sequence that is complementary to at least a portion of the target sequence on the second strand of the target dsDNA molecule with the exception of at least one nucleotide. The template sequence may comprise a nucleotide having a different nucleobase than that of a nucleotide at the corresponding position in the target nucleic acid when the template sequence and the target sequence are aligned for maximum identity. In some embodiments, the primer binding sequence is linked to the template sequence. In some embodiments, the guide RNA and the template RNA are covalently connected. In some embodiments, the guide RNA and the template RNA are not covalently connected, see, e.g. FIGS. 5A-5B. In some embodiments, such compositions comprise an effector protein that is linked to a RDDP. In some embodiments, such compositions comprise an effector protein that is not linked or linked (e.g., covalently linked) to an RDDP. Compositions and systems that comprise an RDDP that is not linked to an effector protein and/or a guide RNA that is not linked to a template RNA may be referred to as a “split protein/RNA design.” See, e.g. FIGS. 5A-5B. In some embodiments, the retRNA comprises a secondary structure. Without being bound by theory, the secondary structure may stabilize the retRNA. In some embodiments, the secondary structure is an aptamer, and the RDDP comprises a peptide that binds the aptamer. The effector protein may nick a strand of the target nucleic acid and the RDDP synthesizes new DNA off the nicked end, wherein the new DNA is complementary to the template sequence (RT template), thereby producing the desired genomic edit in the target nucleic acid. In some embodiments, the effector protein nicks the target strand. In some embodiments, the effector protein nicks the non-target strand.

In some embodiments, compositions and systems comprise a moiety that binds an RNA-aptamer. This moiety may be linked to an RDDP and the RNA-aptamer may be linked to the gRNA or template RNA, or a combination thereof. Thus, the moiety may serve to deliver the RDDP to the target sequence and thus, the RDDP need not be linked to the effector protein. By way of non-limiting example, in some embodiments, compositions and systems comprise an MS2 protein localization sequence. In some embodiments, a guide RNA comprises an MS2 protein localization sequence. In some embodiments, the template RNA comprises an MS2 protein localization sequence. The MS2 protein localization sequence may be located at the 5′ or 3′ terminus of the template RNA. In some embodiments, the RDDP is linked to an MS2 coat protein (or protein that is capable of binding the MS2 protein localization sequence), thereby localizing the RDDP to the MS2 protein localization sequence and therefore, the effector protein, template RNA and/or target nucleic acid. Additional examples of such localizing systems are described by Chen et al., FEBS J. (2013) 280:3734-3754. In some embodiments, an RDDP is linked to an effector protein. In such embodiments, the RDDP may not be fused to an MS2 coat protein and the template RNA may not comprise an MS2 protein localization sequence.

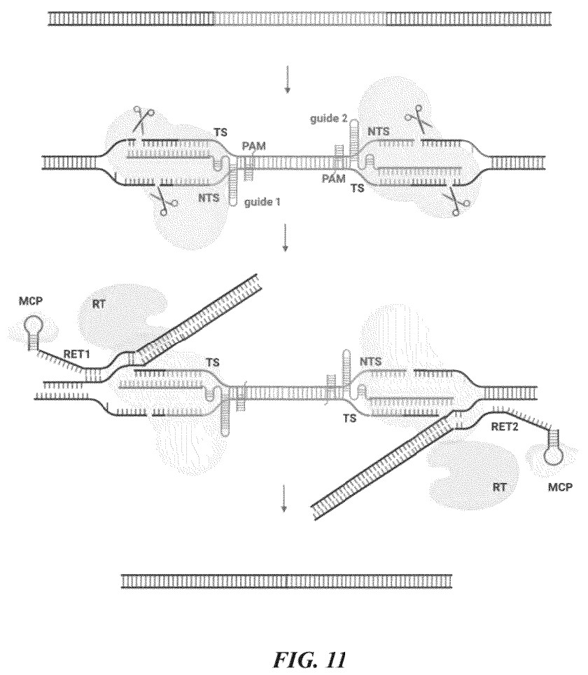

In some embodiments, compositions and systems described herein comprise a homodimerizing effector protein, an RDDP, and two different guide RNAs that hybridize to opposite strands of a dsDNA molecule at some distance apart from each other. In some embodiments, the distance is about 10 bp to about 10,000 bp. In some embodiments, the first target sequence and the second target sequence are greater than 10,000 base pairs apart. The effector proteins form ribonucleoprotein (RNP) complexes with the two guide RNAs at these two different sites respectively where they cleave the dsDNA, generating single stranded DNA (ssDNA) flaps at both sites that extend towards each other as shown in FIG. 11. These systems also comprise two retRNAs, each of which has a primer binding sequence (PBS) that binds to a portion of one of the ssDNA flaps. The retRNAs each have a template sequence for generating a sequence of interest while the region between the two cut sites is deleted.

In some embodiments, the present disclosure provides compositions, systems, and methods for deleting a region of DMD gene. In some embodiments, compositions and systems described herein comprise (a) an effector protein, wherein the effector protein forms a dimer with itself in a cell; (b) an RNA-directed DNA polymerase (RDDP); (c) a first guide RNA (gRNA), wherein the first gRNA comprises (i) a first scaffold sequence, and (ii) a first spacer sequence that hybridizes to a first target sequence on a first strand of the DMD gene, wherein the first scaffold sequence is located 5′ of the first spacer sequence, and wherein the effector protein and the first gRNA form a first RNP complex that cleaves the DMD gene to form a first single stranded DNA (ssDNA) flap from the first strand of the dsDNA target nucleic acid; (d) a second gRNA, wherein the second gRNA comprises (i) a second scaffold sequence, and (ii) a second spacer sequence that hybridizes to a second target sequence on a second strand of the DMD gene, wherein the second scaffold sequence is located 5′ of the second spacer sequence, and wherein the effector protein and the second gRNA form a second RNP complex that cleaves the DMD gene to form a second ssDNA flap from the second strand of the dsDNA target nucleic acid; (e) a first template RNA (retRNA), wherein the first retRNA comprises (i) a first primer binding sequence (PBS) that hybridizes to at least a portion of the first ssDNA flap, and (ii) a first template sequence; and (f) a second retRNA, wherein the second retRNA comprises (i) a second PBS that hybridizes to at least a portion of the second ssDNA flap, and (ii) a second template sequence. See e.g., FIG. 11. In some embodiments, the first spacer sequence and the second spacer sequence hybridize to a first target sequence and a second target sequence of the D) MI) gene respectively. In some embodiments, the first target sequence and the second target sequence are 10 to 10,000 base pairs apart. In some embodiments, the first target sequence and the second target sequence are greater than 10,000 base pairs apart.

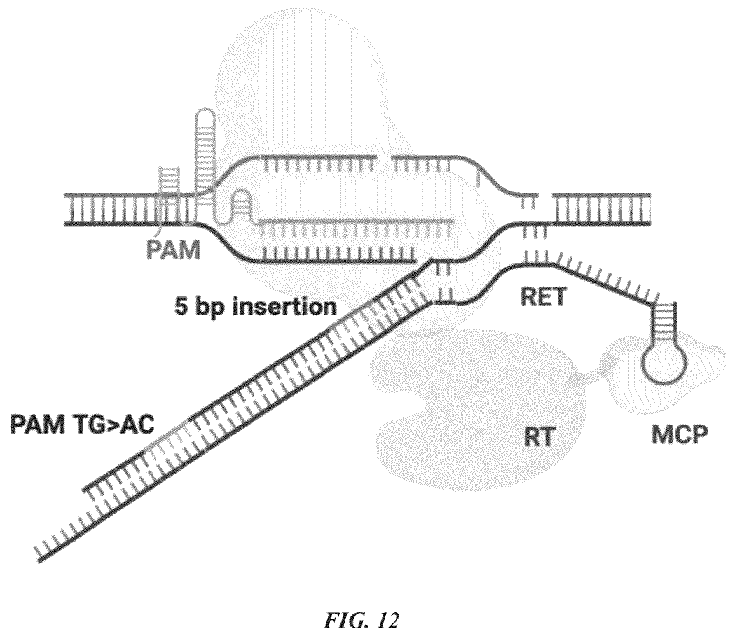

In some embodiments, compositions and systems described herein comprise a homodimerizing effector protein (e.g., a Type V Cas nuclease) that produces a double stranded break, an RDDP, a guide nucleic acid that guides the dimer to a target sequence of a dsDNA target nucleic acid, and a template RNA designed to hybridize to a single stranded DNA from the target strand (TS) to extend the TS. See e.g. FIG. 12. Precision editing with a nuclease and TS extension is different from standard RT editing with a non-target strand (NTS) nickase and NTS extension. In some embodiments, the TS extension with Type V Cas proteins allows editing of the seed region and/or PAM of the TS. In some embodiments, editing of the seed region and/or PAM of the TS prevents subsequent rounds of targeting, thereby reducing the amount of imprecise indels. Non-limiting examples of Type V proteins for such systems include CasM.265466.

In some embodiments, the present disclosure provides compositions, systems, and methods for modifying a target strand (TS) of a human dystrophin gene (I) MI)) gene. In some embodiments, compositions and systems described herein comprise (a) an effector protein, wherein the effector protein forms a dimer with itself in a cell; (b) an RNA-directed DNA polymerase (RDDP); (c) a guide RNA, wherein the guide RNA comprises (i) a first region comprising a scaffold sequence, and (ii) a second region comprising a spacer sequence that hybridizes to a target sequence of the TS of the DMD gene, wherein the first region is located 5′ of the second region; and wherein the effector protein and the gRNA form a RNP complex that produces a double stranded break; and a TS template RNA (retRNA), wherein the TS retRNA comprises a TS primer binding sequence (PBS) that hybridizes to the TS, and a TS template sequence. See e.g., FIG. 12. In some embodiments, the effector protein is a Type V Cas protein. Non-limiting examples of Type V Cas proteins for such systems include CasM.265466.

### Effector Proteins

Provided herein, are compositions, systems, and methods comprising an effector protein and uses thereof. In general, the effector protein is a CRISPR associated (Cas) protein. An effector protein may be a CRISPR-associated (“Cas”) protein. An effector protein may function as a single protein, including a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., modifying a target nucleic acid). An effector protein may be a modified effector protein having increased modification activity and/or increased substrate binding activity (e.g., substrate selectivity, specificity, and/or affinity). Alternatively, or in addition, an effector protein may be a catalytically inactive effector protein having reduced modification activity or no modification activity. Accordingly, an effector protein as used herein encompasses a modified polypeptide that does not have nuclease activity.

An effector protein may be brought into proximity of a target nucleic acid in the presence of a guide nucleic acid when the guide nucleic acid includes a nucleotide sequence that is complementary with a target sequence in the target nucleic acid. The ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid, which may direct the modification activity of the effector protein. An effector protein may modify a target nucleic acid by cis cleavage or trans cleavage. The modification of the target nucleic acid generated by an effector protein may, as a non-limiting example, result in modulation of the expression of the target nucleic acid (e.g., increasing or decreasing expression of the nucleic acid) or modulation of the activity of a translation product of the target nucleic acid (e.g., inactivation of a protein binding to an RNA molecule or hybridization).

In certain embodiments, effector proteins described herein comprise one or more functional domains. Effector protein functional domains can include a protospacer adjacent motif (PAM)-interacting domain, an oligonucleotide-interacting domain, one or more recognition domains, a non-target strand interacting domain, and a RuvC domain. A PAM interacting domain can be a target strand PAM interacting domain (TPID) or a non-target strand PAM interacting domain (NTPID). In some embodiments, a PAM interacting domain, such as a TPID or a NTPID, on an effector protein describes a region of an effector protein that interacts with target nucleic acid. In some embodiments, the effector proteins comprise a RuvC domain. In some embodiments, a RuvC domain comprises with substrate binding activity, catalytic activity, or both. In some embodiments, the RuvC domain may be defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I, RuvC-II, and RuvC-III) that are not contiguous with respect to the primary amino acid sequence of the effector protein, but form a RuvC domain once the protein is produced and folds. In some embodiments, effector proteins comprise one or more recognition domain (REC domain) with a binding affinity for a guide nucleic acid or for a guide nucleic acid-target nucleic acid heteroduplex. An effector protein may comprise a zinc finger domain. In some embodiments, the effector protein does not comprise an HNH domain.

An effector protein may be small, which may be beneficial for nucleic acid detection or editing (for example, the effector protein may be less likely to adsorb to a surface or another biological species due to its small size). The smaller nature of these effector proteins may allow for them to be more easily packaged and delivered with higher efficiency in the context of genome editing and more readily incorporated as a reagent in an assay. In some embodiments, the length of the effector protein is at least 300 linked amino acid residues. In some embodiments, the length of the effector protein is at least 325 linked amino acid residues. In some embodiments, the length of the effector protein is at least 350 linked amino acid residues. In some embodiments, the length of the effector protein is at least 375 linked amino acid residues. In some embodiments, the length of the effector protein is at least 400 linked amino acid residues. In some embodiments, the length of the effector protein is at least 425 linked amino acid residues. In some embodiments, the length of the effector protein is at least 450 linked amino acid residues. In some embodiments, the length of the effector protein is not greater than 700 linked amino acid residues. In some embodiments, the length of the effector protein is not greater than 750 linked amino acid residues. In some embodiments, the length of the effector protein is not greater than 800 linked amino acid residues. In some embodiments, the length of the effector protein is not greater than 850 linked amino acid residues. In some embodiments, the length of the effector protein is not greater than 900 linked amino acid residues. In some embodiments, the length of the effector protein is about 350 to about 450 linked amino acid residues. In some embodiments, the length of the effector protein is about 375 to about 475 linked amino acid residues. In some embodiments, the length of the effector protein is about 400 to about 450 linked amino acid residues. In some embodiments, the length of the effector protein is about 400 to about 500 linked amino acid residues. In some embodiments, the length of the effector protein is about 350 to about 400, about 400 to about 450, about 450 to about 550, about 400 to about 420, about 420 to about 440, about 440 to about 460, about 460 to about 480, about 480 to about 500, about 500 to about 520, about 520 to about 540, about 540 to about 560, about 560 to about 580, about 580 to about 600, about 600 to about 620, about 620 to about 640, about 640 to about 660, about 660 to about 680, about 680 to about 700 linked amino acids. In some embodiments, the length of the effector protein is at least 200, at least 225, at least 250, at least 275 at least 300, at least 325, at least 350, at least 375, at least 400, at least 425, at least 450, at least 475, at least 500, at least 525, at least 550, at least 575, at least 600, at least 625, at least 650, at least 675, at least 700, at least 725, at least 750, at least 775 linked amino acids. In some embodiments, the length of the effector protein is about 700 to about 800 linked amino acid residues.

TABLE 1 provides illustrative amino acid sequences of effector proteins, guide nucleic acid sequences, and PAM sequences that are useful in the compositions, systems and methods described herein. In certain embodiments, compositions, systems, and methods provided herein comprise an effector protein and an engineered guide nucleic acid, wherein the effector protein comprises an amino acid sequence that is at least 65%, at least 70%, at least 75%, at least 80%, at least 85%, at least 90%, at least 95%, at least 98%, at least 99%, or 100% identical to any one of the sequences as set forth in TABLE 1.

In some embodiments, systems and compositions comprise an effector protein and a guide nucleic acid, wherein the effector protein comprises an amino acid sequence that is at least 70%, at least 75%, at least 80%, at least 85%, at least 90%, at least 95%, at least 97%, at least 98%, at least 99%, or 100% identical to SEQ ID NO: 1, and the guide nucleic acid comprises a nucleotide sequence that is at least 70%, at least 75%, at least 80%, at least 85%, at least 90%, at least 95%, at least 97%, at least 98%, at least 99%, or 100% identical to the sequence of SEQ ID NO: 4 or 283.

In some embodiments, systems and compositions comprise an effector protein and a guide nucleic acid, wherein the effector protein comprises an amino acid sequence that is at least 70%, at least 75%, at least 80%, at least 85%, at least 90%, at least 95%, at least 97%, at least 98%, at least 99%, or 100% identical to SEQ ID NO: 2, and the guide nucleic acid comprises a nucleotide sequence that is at least 70%, at least 75%, at least 80%, at least 85%, at least 90%, at least 95%, at least 97%, at least 98%, at least 99%, or 100% identical to the sequence of SEQ ID NO: 6.

In some embodiments, compositions and systems described herein comprise an effector protein that is similar to a naturally occurring effector protein. The effector protein may lack a portion of the naturally occurring effector protein. The effector protein may comprise a mutation relative to the naturally-occurring effector protein, wherein the mutation is not found in nature. The effector protein may also comprise at least one additional amino acid relative to the naturally-occurring effector protein. For example, the effector protein may comprise an addition of a nuclear localization signal relative to the natural occurring effector protein. In certain embodiments, a nucleotide sequence encoding the effector protein is codon optimized (e.g., for expression in a eukaryotic cell) relative to the naturally occurring sequence.

In some instances, effector proteins differ from a sequence in TABLE 1 by one or more amino acids. In some instances, effector proteins differ from a sequence in TABLE 1 by 1 amino acid, 2 amino acids, 3 amino acids, 4, amino acids, 5 amino acids, 6 amino acids, 7 amino acids, 8 amino acids, 9 amino acids or 10 amino acids. In some embodiments up to 15 amino acids are modified. In some embodiments up to 20 amino acids are modified. In some embodiments up to 25 amino acids are modified. Non-limiting examples of effector proteins that have been engineered to differ from a sequence in TABLE 1 are described in TABLE 1.1 and TABLE 1.2.

In some embodiments, the modifications are conservative substitutions relative to the effector protein sequence in TABLE 1. In some embodiments, the amino acids that differ from the effector proteins are non-conservative substitutions relative to the effector protein sequence in TABLE 1. In some embodiments, a mutation may affect the catalytic activity of the effector protein and results in a catalytically reduced or catalytically inactive mutant. In some embodiments, a mutation can result in the effector protein having nickase activity or increased nickase activity. In some embodiments, a mutation can result in the effector protein having reduced or no nuclease activity but gaining nickase activity.

### Protospacer Adjacent Motif (PAM)

Effector proteins of the present disclosure, dimers thereof, and multimeric complexes thereof, may cleave or nick a target nucleic acid within or near a protospacer adjacent motif (PAM) sequence of the target nucleic acid. In some embodiments, cleavage occurs within 1, 2, 3, 4, 5, 6, 7, 8, 9, 10, 11, 12, 13, 14, 15, 16, 17, 18, 19, 20, 21, 22, 23, 24, or 25 nucleotides of a 5′ or 3′ terminus of a PAM sequence. In some embodiments, a target nucleic acid may comprise a PAM sequence adjacent to a target sequence that is complementary to a guide nucleic acid spacer region. PAMs in compositions, systems, and methods herein are further described throughout the application. In some embodiments the PAM is NTTN where N can be any amino acid.

In some embodiments, a target nucleic acid comprises a target sequence that is adjacent to a PAM sequence, wherein the PAM sequence comprises any one of the PAM sequences as set forth in TABLE 1, TABLE 6 and TABLE 8. In some embodiments, systems, compositions, and/or methods described herein comprise a target nucleic acid comprising a target sequence that is adjacent to a PAM sequence, wherein the PAM sequence comprises any one of the PAM sequences as set forth in TABLE 1, TABLE 6 and TABLE 8.

### RNA-Dependent DNA Polymerases (RDDP)

In some embodiments, the present disclosure provides for systems, compositions, and methods comprising an RNA-dependent DNA polymerase (RDDP) or a use thereof. Herein, RDDPs are enzymes that are capable of generating a DNA polynucleotide from an RNA template polynucleotide. In some embodiments, the RDDP is a reverse transcriptase. Herein, the term RDDP encompasses functional domains thereof. In some embodiments, the functional domain is a polymerase domain, an RNAse domain, or a combination thereof.

In some embodiments, the RDDP comprises less than 800 amino acids. In some embodiments, the RDDP comprises less than 800, less than 700, less than 600, less than 500, less than 400, less than 300 amino acids. In some embodiments, the RDDP comprises at least 200, at least 220, at least 240, at least 260, at least 280, or at least 300 amino acids. In some embodiments, the RDDP comprises at least 277 amino acids. In some embodiments, the RDDP comprises 200 to 800 amino acids. In some embodiments, the RDDP comprises 277 to 800 amino acids. In some embodiments, the RDDP comprises 300 to 800, 400 to 800, 500 to 800, or 600 to 800 amino acids. In some embodiments, the RDDP comprises 250 to 750, 250 to 700, 250 to 650, 250 to 600, 250 to 550, 250 to 500, 250 to 450, 250 to 400, 250 to 350, 275 to 750, 275 to 700, 275 to 650, 275 to 600, 275 to 550, 275 to 500, 275 to 450, 275 to 400, 275 to 350, 300 to 750, 300 to 700, 300 to 650, 300 to 600, 300 to 550, 300 to 500, 300 to 450, 300 to 400, or 300 to 350 amino acids.

In some embodiments, the length of the RDDP is less than 800 amino acids. In some embodiments, the length of the RDDP is less than 800, less than 700, less than 600, less than 500, less than 400, less than 300 linked amino acids. In some embodiments, the length of the RDDP is at least 200, at least 220, at least 240, at least 260, at least 280, or at least 300 linked amino acids. In some embodiments, the length of the RDDP is at least 277 linked amino acids. In some embodiments, the length of the RDDP is 200 to 800 linked amino acids. In some embodiments, the length of the RDDP is 277 to 800 linked amino acids. In some embodiments, the length of the RDDP is 300 to 800, 400 to 800, 500 to 800, or 600 to 800 linked amino acids. In some embodiments, the length of the RDDP is 250 to 750, 250 to 700, 250 to 650, 250 to 600, 250 to 550, 250 to 500, 250 to 450, 250 to 400, 250 to 350, 275 to 750, 275 to 700, 275 to 650, 275 to 600, 275 to 550, 275 to 500, 275 to 450, 275 to 400, 275 to 350, 300 to 750, 300 to 700, 300 to 650, 300 to 600, 300 to 550, 300 to 500, 300 to 450, 300 to 400, or 300 to 350 linked amino acids.

In some embodiments, the RDDP, when used in combination with an effector protein described herein, demonstrates an editing efficiency of at least 1%. In some embodiments, the RDDP, when used in combination with an effector protein described herein, demonstrates an editing efficiency of at least 2%, 3%, 4%, 5%, 6%, 7%, 8%, 9%, or at least 10%. In some embodiments, the RDDP, when used in combination with an effector protein described herein, demonstrates an editing efficiency that is greater than the editing efficiency observed with a Moloney murine leukemia virus (M-MLV) reverse transcriptase (e.g., SEQ ID NO: 1592 or 1593). In some embodiments, the RDDP, when used in combination with an effector protein described herein, demonstrates an editing efficiency that is at least 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or at least 10-fold greater than the editing efficiency observed with an M-MLV reverse transcriptase.

In some embodiments, the RDDP comprises an amino acid sequence that it at least 90%, 91%, 92%, 93%, 94%, 95%, 96%, 97%, 98%, or 99% identical to one of SEQ ID NOs: 11-79. In some embodiments, the RDDP comprises or consists of an amino sequence selected from SEQ ID NOs: 11-79.

In some embodiments, the RDDP comprises an amino acid sequence that it at least 90%, 91%, 92%, 93%, 94%, 95%, 96%, 97%, 98%, or 99% identical to SEQ ID NO: 22. In some embodiments, the RDDP comprises or consists of the amino sequence of SEQ ID NO: 22. In some embodiments, the RDDP comprises an amino acid sequence that it at least 90%, 91%, 92%, 93%, 94%, 95%, 96%, 97%, 98%, or 99% identical to SEQ ID NO: 24. In some embodiments, the RDDP comprises or consists of the amino sequence of SEQ ID NO: 24. In some embodiments, the RDDP comprises an amino acid sequence that it at least 90%, 91%, 92%, 93%, 94%, 95%, 96%, 97%, 98%, or 99% identical to SEQ ID NO: 30. In some embodiments, the RDDP comprises or consists of the amino sequence of SEQ ID NO: 30. In some embodiments, the RDDP comprises an amino acid sequence that it at least 90%, 91%, 92%, 93%, 94%, 95%, 96%, 97%, 98%, or 99% identical to SEQ ID NO: 32. In some embodiments, the RDDP comprises or consists of the amino sequence of SEQ ID NO: 32. In some embodiments, the RDDP comprises an amino acid sequence that it at least 90%, 91%, 92%, 93%, 94%, 95%, 96%, 97%, 98%, or 99% identical to SEQ ID NO: 40. In some embodiments, the RDDP comprises or consists of the amino sequence of SEQ ID NO: 40.

In some embodiments, the present disclosure provides a polynucleotide encoding an RDDP described herein. In some embodiments, the present disclosure provides a polynucleotide encoding an RDDP comprising an amino acid sequence that it at least 90%, 91%, 92%, 93%, 94%, 95%, 96%, 97%, 98%, or 99% identical to one of SEQ ID NOs: 11-79. In some embodiments, the polynucleotide encodes an RDDP comprising or consisting of an amino sequence selected from SEQ ID NOs: 11-79. Exemplary RDDP sequences are provided in TABLE 3.

In some instances, RDDPs comprise at least 200, at least 225, at least 250, at least 275 at least 300, at least 325, at least 350, at least 375, at least 400, at least 425, at least 450, at least 475, at least 500, at least 525, at least 550, at least 575, at least 600, at least 625, at least 650, at least 675, at least 700, at least 725, at least 750, at least 775 contiguous amino acids of a sequence selected from SEQ ID NOs: 11-79.

### Engineered Proteins

In another example, any of the protein sequences herein may be codon optimized. In some embodiments, effector protein described herein are encoded by a codon optimized nucleic acid. In some embodiments, a nucleic acid sequence encoding an effector protein described herein, is codon optimized. This type of optimization can entail a mutation of an effector protein encoding nucleotide sequence to mimic the codon preferences of the intended host organism or cell while encoding the same polypeptide. Thus, the codons can be changed, but the encoded protein remains unchanged. For example, if the intended target cell was a human cell, a human codon-optimized effector protein-encoding nucleotide sequence could be used. As another non-limiting example, if the intended host cell were a mouse cell, then a mouse codon-optimized effector protein-encoding nucleotide sequence could be generated. As another non-limiting example, if the intended host cell were a eukaryotic cell, then a eukaryote codon-optimized effector protein nucleotide sequence could be generated. As another non-limiting example, if the intended host cell were a prokaryotic cell, then a prokaryote codon-optimized effector protein-encoding nucleotide sequence could be generated. Codon usage tables are readily available, for example, at the “Codon Usage Database” available at www.kazusa.or.jp/codon. Accordingly, in some embodiments, effector proteins described herein may be codon optimized for expression in a specific cell, for example, a bacterial cell, a plant cell, a eukaryotic cell, an animal cell, a mammalian cell, or a human cell. In some embodiments, the effector protein is codon optimized for a human cell.

It is understood that when describing coding sequences of polypeptides described herein, said coding sequences do not necessarily require a codon encoding a N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some embodiments, when a modifying heterologous peptide, such as a fusion partner protein, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

In some embodiments, the RDDP and/or effector proteins described herein comprise one or more amino acid substitutions as compared to a naturally occurring RDDP and/or effector protein. In some embodiments, the amino acid substitution is a conservative amino acid substitution. In general, a conservative amino acid substitution is the substitution of an amino acid residue for another amino acid residue with similar chemical properties (e.g., size, charge, or polarity). Conservative substitutions may be made by exchanging an amino acid from one of the groups listed below (group 1 to 6) for another amino acid of the same group.

Amino acid residues may be divided into groups based on common side chain properties, as follows: (group 1) hydrophobic: norleucine, methionine (Met), Alanine (Ala), Valine (Val), Leucine (Leu), Isoleucine (Ile); (group 2) neutral hydrophilic: Cysteine (Cys), Serine (Ser), Threonine (Thr), Asparagine (Asn), Glutamine (Gln); (group 3) acidic: Aspartic acid (Asp), Glutamic acid (Glu); (group 4) basic: Histidine (His), Lysine (Lys), Arginine (Arg); (group 5) residues that influence chain orientation: Glycine (Gly), Proline (Pro); and (group 6) aromatic: Tryptophan (Trp), Tyrosine (Tyr), Phenylalanine (Phe). The substitution of one amino acid with another amino acid in a same group listed above may be considered a conservative amino acid substitution. The substitution of one amino acid with another amino acid in a different group listed above may be considered a non-conservative amino acid substitution.

In some embodiments, the effector protein is an engineered effector protein and comprises an amino acid sequence that is at least 90%, at least 95%, at least 97%, at least 98%, or at least 99% identical to SEQ ID NO: 1, wherein the polypeptide comprises at least one amino acid substitution relative to SEQ ID NO: 1, wherein the amino acid substitution is at a position selected from K58, 180, T84, K105, N193, C202, S209. G210, A218. D220. E225. C246, N286. M295, M298, A306, Y315, Q360, and a combination thereof. In some embodiments, the polypeptide comprises an amino acid sequence that is 100% identical to SEQ ID NO: 1, with the exception of at least one amino acid substitution relative to SEQ ID NO: 1, wherein the amino acid substitution is a position selected from K58, 180, T84, K105, N193, C202, S209, G210, A218, D220, E225, C246, N286, M295, M298, A306, Y315, Q360, and a combination thereof. In some embodiments, the amino acid substitution is selected from K58X, 180X, T84X, K105X, N193X, C202X, S209X, G210X, A218X, D220X, E225X, C246X, N286X, M295X, M298X, A306X, Y315X, and Q360X, wherein X is selected from R, K, and H.

In some embodiments, the effector protein is an engineered effector protein and comprises an amino acid sequence that is at least 90%, at least 95%, at least 97%, at least 98%, or at least 99% identical to SEQ ID NO: 1, wherein the polypeptide comprises at least one amino acid substitution relative to SEQ ID NO: 1, wherein the amino acid substitution is selected from 180R, T84R, K105R, C202R, G210R, A218R, D220R, E225R, C246R, Q360R, 180K, T84K, G210K, N193K, C202K, A218K, D220K, E225K, C246K, N286K, A306K, Q360K, 180H, T84H, K105H, G210H, C202H, A218H, D220H, E225H, C246H, Q360H, K58W, S209F, M295W, M298L, Y315M, D22OR and A306K, D220R and K250N, and a combination thereof. In some embodiments, the polypeptide comprises an amino acid sequence that is 100% identical to SEQ ID NO: 1, with the exception of at least one amino acid substitution relative to SEQ ID NO: 1, wherein the amino acid substitution is selected from I80R, T84R, KI05R, C202R, G210R, A218R, D220R, E225R, C246R, Q360R, 180K, T84K, G210K, N193K, C202K, A218K, D220K, E225K, C246K, N286K, A306K, Q360K, 180H, T84H, K105H, G210H, C202H, A218H, D220H, E225H, C246H, Q360H, K58W, S209F, M295W, M298L, Y315M, D220R and A306K, D220R and K250N, and a combination thereof. In some embodiments, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 1, wherein the polypeptide comprises at least one amino acid substitution relative to SEQ ID NO: 1, wherein the amino acid substitution is selected from D237A, D418A, D418N, E335A, and E335Q, and a combination thereof. In some embodiments, the polypeptide comprises an amino acid sequence that is 100% identical to SEQ ID NO: 1, with the exception of at least one amino acid substitution relative to SEQ ID NO: 1, wherein the amino acid substitution is selected from D237A, D418A, D418N, E335A, and E335Q, and a combination thereof. In some aspects, these engineered effector proteins demonstrate reduced or abolished nuclease activity relative to the wild-type effector protein. TABLE 1.1 provides the exemplary amino acid alterations relative to SEQ ID NO: 1 useful in compositions, systems, and methods described herein.

In some embodiments, the effector protein is an engineered effector protein and comprises an amino acid sequence that is at least 90%, at least 95%, at least 97%, at least 98%, or at least 99% identical to SEQ ID NO: 2, wherein the polypeptide comprises at least one amino acid substitution relative to SEQ ID NO: 2, wherein the amino acid substitution is at a position selected from 12, T5, K15, R18, H20, S21, L26, N30, E33, E34, A35, K37, K38, R41, N43, Q54, Q79R, K92E, K99R, S108, E109, H110, G111, D113, T114, P116, K118, E119, A121, N132, K135, Q138, V139, N148, L149, E157, E164, E166, E170, Y180, L182, Q183, K184, S186, K189, S196, S198, K200, 1203, S205, K206, Y207, H208, N209, Y220, S223, E258, K281, K348, N355, S362, N406, K435, I471, I489, Y490, F491, D495, K496, K498, K500, D501, V502, K504, S505, D506, V521, N568, S579, Q612, S638, F701, P707, and a combination thereof. In some embodiments, the polypeptide comprises an amino acid sequence that is 100% identical to SEQ ID NO: 2, with the exception of at least one amino acid substitution relative to SEQ ID NO: 2, wherein the amino acid substitution is at a position selected from 12, T5, K15, R18, H20, S21, L26, N30, E33, E34, A35, K37, K38, R41, N43, Q54, Q79R, K92E, K99R, S108, E109, H110, G111, D113, T114, P116, K118, E119, A121, N132, K135, Q138, V139, N148, L149, E157, E164, E166, E170, Y180, L182, Q183, K184, S186, K189, S196, S198, K200, 1203, S205, K206, Y207, H208, N209, Y220, S223, E258, K281, K348, N355, S362, N406, K435, I471, I489, Y490, F491, D495, K496, K498, K500, D501, V502, K504, S505, D506, V521, N568, S579, Q612, S638, F701, P707, and a combination thereof. In some embodiments, the amino acid substitution is selected from I2X, T5X, K15X, R18X, H20X, S21X, L26X, N30X, E33X, E34X, A35X, K37X, K38X, R41X, N43X, Q54X, Q79RX, K92EX, K99RX, S108X, E109X, H110X, G111X, D113X, T114X, P116X, K118X, E119X, A121X, N132X, K135X, Q138X, V139X, N148X, L149X, E157X, E164X, E166X, E170X, Y180X, L182X, Q183X, K184X, S186X, K189X, S196X, S198X, K200X, 1203X, S205X, K206X, Y207X, H208X, N209X, Y220X, S223X, E258X, K281X, K348X, N355X, S362X, N406X, K435X, I471X, I489X, Y490X, F491X. D495X, K496X, K498X, K500X, D501X, V502X, K504X, S505X, D506X, V521X, N568X, S579X, Q612X, S638X, F701X, P707X, wherein X is selected from R, K, and H.

In some embodiments, the effector protein is an engineered effector protein and comprises an amino acid sequence that is at least 90%, at least 95%, at least 97%, at least 98%, or at least 99% identical to SEQ ID NO: 2, wherein the polypeptide comprises at least one amino acid substitution relative to SEQ ID NO: 2 wherein the amino acid substitution is selected from T5R, L26X, L26K, A121Q, V139R, N148R, S198R, H208R, S223P, E258K, N355R, I471T, S579R, F701R, D703G, P707R, K189P, S638K, Q54R, Q79R, Y220S, N406K, E119S, K92E, K435Q, N568D, and V521T, and a combination thereof. In some embodiments, the polypeptide comprises an amino acid sequence that is 100% identical to SEQ ID NO: 2, with the exception of at least one amino acid substitution relative to SEQ ID NO: 2, wherein the amino acid substitution is selected from T5R, L26X, L26K, A121Q, V139R, N148R, S198R, H208R, S223P, E258K, N355R, 1471T, S579R, F701R, P707R, D703G, K189P, S638K, Q54R, Q79R, Y220S, N406K, E119S, K92E, K435Q, N568D, and V521T, and a combination thereof. In some embodiments, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 2, wherein the polypeptide comprises at least two amino acid substitutions relative to SEQ ID NO: 2, wherein the at least two amino acid substitutions are selected from L26K and A121Q, L26X and A121Q, K99R and L149R, K99R and N148R, L149R and H208R, S362R and L26X, L26X and N148R, L26X and H208R, N30R and N148R, L26X and K99R, L26X and P707R, L26X and L149R, L26X and N30R, L26X and N355R, L26X and K281R, L26X and S108R, L26X and K348R, T5R and V139R, I2R and V139R, K99R and S186R, L26X and A673G, L26X and Q674R, S579R and L26K, F701R and E258K, T5R and L26K, L26X and K435Q, L26X and G685R, L26X and Q674K, L26X and P699R, L26X and T70E, L26X and Q232R, L26X and T252R, L26X and P679R, L26X and E83K, L26X and E73P, L26X and K248E, L26X, T5R and S223P, S579R and S223P, L26X and S223P, T5R and A121Q, L26X and A696R, S198R and I471T, L26X and N153R, L26X and E682R, L26X and D703R, Q612R and L26K, L26X and 1471T, K348R and L26K, S579R and 1471T, L26X and V228R, T5R and S638K, S579R and K189P, S579R and E258K, L26X and K260R, L26X and S638K, S579R and Y220S, T5R and 1471T, L26X and F233R, L26X and V521T, F701R and A12IQ, L26X and G361R, S198R and E258K, L26X and S472R, T5R and Y220S, L26X and A150K, L26X and S684R, L26X and E157R, L26X and K248R, F701R and L26K, S198R and N406K, S198R and Y220S, S198R and S638K, S198R and V521T, S579R and A121Q, K348R and Y220S, S198R and K189P, L26X and E242R, L26X and K678R, T5R and N406K, L26X and 1158K, T5R and V521T, L26X and N259R, L26X and K257R, L26X and K256R, T5R and K189P, L26X and C405R, S579R and V521T, S579R and N406K, T5R and K92E, T5R and E258K, L26X and 197R, S579R and S638K, T5R and K435Q, F701R and S638K, L26X and L236R, F701R and 1471T, Q612R and S223P, F701R and S223P, S198R and E119S, S579R and K92E, L26X and E715R, Q612R and 1471T, F701R and Y220S, S198R and S223P, and L26X and K266R, and a combination thereof, wherein X is selected from R and K. In some embodiments, the polypeptide comprises an amino acid sequence that is 100% identical to SEQ ID NO: 2, with the exception of at least one amino acid substitution relative to SEQ ID NO: 2, wherein the amino acid substitution is selected from N148R, H208R, N355R, L26K and A121Q, L26X and A121Q, K99R and L149R, K99R and N148R, L149R and H208R, S362R and L26X, L26X and N148R, L26X and H208R, N30R and N148R, L26X and K99R, L26X and P707R, L26X and L149R, L26X and N30R, L26X and N355R, L26X and K281R, L26X and S108R, L26X and K348R, T5R and V139R, 12R and V139R, K99R and S186R, L26X and A673G, L26X and Q674R, S579R and L26K, F701R and E258K, T5R and L26K, L26X and K435Q, L26X and G685R, L26X and Q674K, L26X and P699R, L26X and T70E, L26X and Q232R, L26X and T252R, L26X and P679R, L26X and E83K, L26X and E73P, L26X and K248E, L26X, T5R and S223P, S579R and S223P, L26X and S223P, T5R and A121Q, L26X and A696R, S198R and 1471T, L26X and N153R, L26X and E682R, L26X and D703R, Q612R and L26K, L26X and I471T, K348R and L26K, S579R and I471T, L26X and V228R, T5R and S638K, S579R and K189P, S579R and E258K, L26X and K260R, L26X and S638K, S579R and Y220S, T5R and 1471T, L26X and F233R, L26X and V521T, F701R and A121Q, L26X and G361R, S198R and E258K, L26X and S472R, T5R and Y220S, L26X and A150K, L26X and S684R, L26X and E157R, L26X and K248R, F701R and L26K, S198R and N406K, S198R and Y220S, S198R and S638K, S198R and V521T, S579R and A121Q, K348R and Y220S, S198R and K189P, L26X and E242R, L26X and K678R, T5R and N406K, L26X and 1158K, T5R and V521T, L26X and N259R, L26X and K257R, L26X and K256R, T5R and K189P, L26X and C405R, S579R and V521T, S579R and N406K, T5R and K92E, T5R and E258K, L26X and 197R, S579R and S638K, T5R and K435Q, F701R and S638K, L26X and L236R, F701R and 147IT, Q612R and S223P. F701R and S223P, S198R and E119S, S579R and K92E, L26X and E715R, Q612R and I471T, F701R and Y220S, S198R and S223P, and L26X and K266R, and a combination thereof, wherein X is selected from R and K. In some embodiments, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 2, wherein the effector protein comprises four amino acid substitutions relative to SEQ ID NO: 2, wherein the amino acid substitutions comprise L26R, I471T, S223P, and D703G. In some embodiments, the effector protein comprises an amino acid sequence that is 100% identical to SEQ ID NO: 2, with the exception of four amino acid substitutions relative to SEQ ID NO: 2, wherein the amino acid substitutions comprise L26R, I471T, S223P, and D703G. In some embodiments, the effector protein comprises an amino acid sequence that is at least 90%, at least 95%, at least 97%, at least 98%, at least 99%, or 100% identical to SEQ ID NO: 1607. In some embodiments, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 2, wherein the polypeptide comprises at least one amino acid substitution relative to SEQ ID NO: 2 wherein the amino acid substitution is selected from E157A, E164A, E164L, E166A, E166I, E170A, 1489A, I489S, Y490S, Y490A, F491A, F491S, F491G, D495G, D495R, D495K. K496A, K496S, K498A, K498S, K500A, K500S, D501R, D501G, D501K, V502A, V502S, K504A, K504S, S505R, D506A, and a combination thereof. In some embodiments, the polypeptide comprises an amino acid sequence that is 100% identical to SEQ ID NO: 2, with the exception of at least one amino acid substitution relative to SEQ ID NO: 2, wherein the amino acid substitution is selected from E157A, E164A, E164L, E166A, E166I, E170A, 1489A, 1489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R, D506A, and a combination thereof. In some embodiments, these engineered effector proteins comprise a nickase activity.

In some embodiments, the effector protein is an engineered effector protein and comprises an amino acid sequence that is at least 90%, at least 95%, at least 97%, at least 98%, or at least 99% identical, or is 100% identical to SEQ ID NO: 2, wherein amino acids S478-S505 have been deleted. In some embodiments, the effector protein is an engineered effector protein and comprises an amino acid sequence that is at least 90%, at least 95%, at least 97%, at least 98%, or at least 99% identical, or is 100% identical to SEQ ID NO: 2, wherein amino acids S478-S505 have been deleted and replaced with SDLYIERGGDPRDVHQQVETKPKGKRKSEIRILKIR (SEQ ID NO: 7) or SDYIVDHGGDPEKVFFETKSKKDKTKRYKRR (SEQ ID NO: 8). In some embodiments, the effector protein is an engineered effector protein and comprises an amino acid sequence that is at least 90%, at least 95%, at least 97%, at least 98%, at least 99% identical, or is 100% identical to SEQ ID NO: 9. In some embodiments, the effector protein is an engineered effector protein and comprises an amino acid sequence that is at least 90%, at least 95%, at least 97%, at least 98%, at least 99% identical, or is 100% identical to SEQ ID NO: 10.

In some embodiments, the effector protein is an engineered effector protein and comprises an amino acid sequence that is at least 90%, at least 95%, at least 97%, at least 98%, or at least 99% identical to SEQ ID NO: 2, wherein the polypeptide comprises at least one amino acid substitution relative to SEQ ID NO: 2 wherein the amino acid substitution is selected from D369A, D369N, D658A, D658N, E567A, E567Q, and a combination thereof. In some embodiments, the polypeptide comprises an amino acid sequence that is 100% identical to SEQ ID NO: 2, with the exception of at least one amino acid substitution relative to SEQ ID NO: 2, wherein the amino acid substitution is selected from D369A, D369N, D658A, D658N, E567A, E567Q, and a combination thereof. In some embodiments, these engineered effector proteins demonstrate reduced or abolished nuclease activity relative to the wild-type effector protein. TABLE 1.2 provides the exemplary amino acid alterations relative to SEQ ID NO: 2 useful in compositions, systems, and methods described herein.

In some embodiments, the RDDP is an engineered RDDP and comprises an amino acid sequence that it at least 90%, 91%, 92%, 93%, 94%, 95%, 96%, 97%, 98%, or 99% identical to one of SEQ ID NOs: 80-89. In some embodiments, the RDDP comprises or consists of an amino sequence selected from SEQ ID NOs: 80-89.

In some embodiments, the present disclosure provides a polynucleotide encoding an RDDP described herein. In some embodiments, the present disclosure provides a polynucleotide encoding an RDDP comprising an amino acid sequence that it at least 90%, 91%, 92%, 93%, 94%, 95%, 96%, 97%, 98%, or 99% identical to one of SEQ ID NOs: 80-89. In some embodiments, the polynucleotide encodes an RDDP comprising or consisting of an amino sequence selected from SEQ ID NOs: 80-89.

In some instances, RDDPs comprise at least 200, at least 225, at least 250, at least 275 at least 300, at least 325, at least 350, at least 375, at least 400, at least 425, at least 450, at least 475, at least 500, at least 525, at least 550, at least 575, at least 600, at least 625, at least 650, at least 675, at least 700, at least 725, at least 750, at least 775 contiguous amino acids of a sequence selected from SEQ ID NOs: 80-89.

Exemplary engineered RDDPs are provided in TABLE 4. For each engineered RDDP, the parental RDDP is listed followed by the amino acid mutations in parentheses. For example, 2691319 (D12R-D72R-N195R) (SEQ ID NO: 80) refers to an engineered RDDP based on 2691319 (SEQ ID NO: 22) and comprising the mutations D12R, D72R, and N195R.

Engineered effector proteins may provide enhanced catalytic activity (e.g., nuclease or nickase activity) as compared to a naturally occurring nuclease or nickase. Engineered effector proteins may provide enhance nucleic acid binding activity, e.g., enhanced binding of a guide nucleic acid and/or target nucleic acid, and/or may demonstrate a stronger affinity for a target nucleic acid sequence. Without wishing to be bound by theory, substation of positively charged amino acids is thought to increase the interaction between the effector proteins and/or RDDPs and the negatively charged target nucleic acid sequences. By way of non-limiting example, some engineered proteins exhibit optimal activity at lower salinity and viscosity than the protoplasm of their bacterial cell of origin. Also, by way of non-limiting example, bacteria often comprise protoplasmic salt concentrations greater than 250 mM and room temperature intracellular viscosities above 2 centipoise, whereas engineered proteins exhibit optimal activity (e.g., cis-cleavage activity) at salt concentrations below 150 mM and viscosities below 1.5 centipoise. The present disclosure leverages these dependencies by providing engineered proteins in solutions optimized for their activity and stability.

Compositions and systems described herein may comprise an engineered effector protein and/or RDDP in a solution comprising a room temperature viscosity of less than about 15 centipoise, less than about 12 centipoise, less than about 10 centipoise, less than about 8 centipoise, less than about 6 centipoise, less than about 5 centipoise, less than about 4 centipoise, less than about 3 centipoise, less than about 2 centipoise, or less than about 1.5 centipoise.

Compositions and systems may comprise an engineered effector protein and/or RDDP in a solution comprising an ionic strength of less than about 500 mM, less than about 400 mM, less than about 300 mM, less than about 250 mM, less than about 200 mM, less than about 150 mM, less than about 100 mM, less than about 80 mM, less than about 60 mM, or less than about 50 mM. Compositions and systems may comprise an engineered effector protein and/or RDDP and an assay excipient, which may stabilize a reagent or product, prevent aggregation or precipitation, or enhance or stabilize a detectable signal (e.g., a fluorescent signal). Examples of assay excipients include, but are not limited to, saccharides and saccharide derivatives (e.g., sodium carboxymethyl cellulose and cellulose acetate), detergents, glycols, polyols, esters, buffering agents, alginic acid, and organic solvents (e.g., DMSO).

An engineered protein may comprise a modified form of a wild type counterpart protein (e.g., an effector protein and/or RDDP). The modified form of the wild type counterpart may comprise an amino acid change (e.g., deletion, insertion, or substitution) that reduces the nucleic acid-cleaving activity of the effector protein relative to the wild type counterpart. For example, a nuclease domain (e.g., RuvC domain) of an effector protein may be deleted or mutated relative to a wild type counterpart effector protein so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. Engineered effector proteins may have no substantial nucleic acid-cleaving activity. An engineered effector protein may be enzymatically inactive or “dead,” also referred to in some instances as a dead protein or a dCas protein. An engineered effector protein may bind to a guide nucleic acid and/or a target nucleic acid but not cleave the target nucleic acid. An enzymatically inactive effector protein may comprise an enzymatically inactive domain (e.g., inactive nuclease domain). Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with a guide nucleic acid to activate or repress transcription of a target nucleic acid. In some embodiments, the enzymatically inactive protein is linked to a fusion partner protein that confers an alternative activity to an effector protein activity. Such fusion proteins are described herein and throughout. By way of non-limiting example, an alternative activity may be a transcriptional activation, transcription repression, deaminase activity, transposase activity, and recombinase activity. In some embodiments, activity (e.g., nuclease activity) of effector proteins and/or compositions described herein can be measured relative to a WT effector protein or compositions containing the same in a cleavage assay.

### Modulation of Effector Protein Activity

The effector proteins of the present disclosure may have nuclease activity, nickase activity, or no cleavage activity on target nucleic acids. In some embodiments the effector protein of the present disclosure has a combination of the above activities on a target nucleic acid. In some embodiments the cleavage activity of the effector proteins is modulated between nuclease activity and nickase activity on the target nucleic acid. In some embodiments the cleavage activity of the effector protein is modulated between nickase activity and no cleavage activity on the target nucleic acid. In some embodiments the cleavage activity of the effector protein is modulated between nuclease activity and no cleavage activity on the target nucleic acid. In some embodiments the cleavage activity of the effector protein is modulated between nuclease activity, nickase activity, and no cleavage activity on the target nucleic acid. In some embodiments the effector protein has nuclease activity. In some embodiments the effector protein has nickase activity.

In some embodiments the cleavage activity of the effector protein on the target nucleic acid is modulated based on the length of the spacer sequence of a guide nucleic acid. In some embodiments the spacer length confers nuclease activity to the effector protein on the target nucleic acid. In some embodiments the spacer length confers nickase activity to the effector protein on the target nucleic acid. In some embodiments the spacer length confers no cleavage activity to the effector protein on the target nucleic acid. In some embodiments, the spacer length is 10-20 nucleotides. In some embodiments, the spacer length is 11-17 nucleotides. In some embodiments, the spacer length is 14-16 nucleotides. In some embodiments, the spacer length is 10, 12, 13, 14, 15, 16 or 17 nucleotides. In some embodiments, the spacer length is 15 nucleotides.

### Fusion Proteins

In some embodiments, the present disclosure provides a fusion protein comprising an effector protein described herein and an RDDP described herein. In some embodiments, the fusion protein comprises, from N-terminus to C-terminus, an effector protein and an RDDP. In some embodiments, the fusion protein comprises, from N-terminus to C-terminus, an RDDP and an effector protein. Exemplary fusion protein sequences are provided in TABLE 5.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1 and 2 and an RDDP comprising an amino acid sequence that is at least 90%, 91%, 92%, 93%, 94%, 95%, 96%, 97%, 98%, or 99% identical to an amino acid sequence selected from SEQ ID NOs: 11-79. In some embodiments, the fusion protein described herein comprises an effector protein comprising or consisting of an amino acid sequence selected from SEQ ID NOs: 1 and 2 and an RDDP comprising or consisting of an amino acid sequence selected from SEQ ID NOs: 11-79. In some embodiments, the effector protein comprises at least one amino acid alteration relative to the amino acid sequence of SEQ ID NO: 1. In some embodiments, the at least one amino acid alteration comprises an amino acid alteration set forth in TABLE 1.1. In some embodiments, the effector protein comprises at least one amino acid alteration relative to the amino acid sequence of SEQ ID NO: 2. In some embodiments, the at least one amino acid alteration comprises an amino acid alteration set forth in TABLE 1.2.

In some embodiments, the fusion protein described herein comprises an effector protein comprising an amino acid sequence that is at least 90%, 91%, 92%, 93%, 94%, 95%, 96%, 97%,98%, or 99% identical to an amino acid sequence selected from SEQ ID NOs: 1 and 2 and an RDDP comprising an amino acid sequence that is at least 90%, 91%, 92%, 93%, 94%, 95%, 96%, 97%,98%, or 99% identical to SEQ ID NO: 22. In some embodiments, the fusion protein described herein comprises an effector protein comprising or consisting of an amino acid sequence selected from SEQ ID NOs: 1 and 2 and an RDDP comprising or consisting of the amino acid sequence of SEQ ID NO: 22. In some embodiments, the fusion protein comprises an amino acid sequence that is at least 90%, 91%, 92%, 93%, 94%, 95%, 96%, 97%, 98%, or 99% identical to SEQ ID NO: 90 or 95. In some embodiments, the fusion protein comprises or consists of SEQ ID NO: 90 or 95. In some embodiments, the effector protein comprises at least one amino acid alteration relative to the amino acid sequence of SEQ ID NO: 1. In some embodiments, the at least one amino acid alteration comprises an amino acid alteration set forth in TABLE 1.1. In some embodiments, the effector protein comprises at least one amino acid alteration relative to the amino acid sequence of SEQ ID NO: 2. In some embodiments, the at least one amino acid alteration comprises an amino acid alteration set forth in TABLE 1.2.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1 and 2 and an RDDP comprising an amino acid sequence that is at least 90%, 91%, 92%, 93%, 94%, 95%, 96%, 97%, 98%, or 99% identical to SEQ ID NO: 24. In some embodiments, the fusion protein described herein comprises an effector protein comprising or consisting of an amino acid sequence selected from SEQ ID NOs: 1 and 2 and an RDDP comprising or consisting of the amino acid sequence of SEQ ID NO: 24. In some embodiments, the fusion protein comprises an amino acid sequence that is at least 90%, 91%, 92%, 93%, 94%, 95%, 96%, 97%, 98%, or 99% identical to SEQ ID NO: 91 or 96. In some embodiments, the fusion protein comprises or consists of SEQ ID NO: 91 or 96. In some embodiments, the effector protein comprises at least one amino acid alteration relative to the amino acid sequence of SEQ ID NO: 1. In some embodiments, the at least one amino acid alteration comprises an amino acid alteration set forth in TABLE 1.1. In some embodiments, the effector protein comprises at least one amino acid alteration relative to the amino acid sequence of SEQ ID NO: 2. In some embodiments, the at least one amino acid alteration comprises an amino acid alteration set forth in TABLE 1.2.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1 and 2 and an RDDP comprising an amino acid sequence that is at least 90%, 91%, 92%, 93%, 94%, 95%, 96%, 97%, 98%, or 99% identical to SEQ ID NO: 30. In some embodiments, the fusion protein described herein comprises an effector protein comprising or consisting of an amino acid sequence selected from SEQ ID NOs: 1 and 2 and an RDDP comprising or consisting of the amino acid sequence of SEQ ID NO: 30. In some embodiments, the fusion protein comprises an amino acid sequence that is at least 90%, 91%, 92%, 93%, 94%, 95%, 96%, 97%, 98%, or 99% identical to SEQ ID NO: 92 or 97. In some embodiments, the fusion protein comprises or consists of SEQ ID NO: 92 or 97. In some embodiments, the effector protein comprises at least one amino acid alteration relative to the amino acid sequence of SEQ ID NO: 1. In some embodiments, the at least one amino acid alteration comprises an amino acid alteration set forth in TABLE 1.1. In some embodiments, the effector protein comprises at least one amino acid alteration relative to the amino acid sequence of SEQ ID NO: 2. In some embodiments, the at least one amino acid alteration comprises an amino acid alteration set forth in TABLE 1.2.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1 and 2 and an RDDP comprising an amino acid sequence that is at least 90%, 91%, 92%, 93%, 94%, 95%, 96%, 97%, 98%, or 99% identical to SEQ ID NO: 32. In some embodiments, the fusion protein described herein comprises an effector protein comprising or consisting of an amino acid sequence selected from SEQ ID NOs: 1 and 2 and an RDDP comprising or consisting of the amino acid sequence of SEQ ID NO: 32. In some embodiments, the fusion protein comprises an amino acid sequence that is at least 90%, 91%, 92%, 93%, 94%, 95%, 96%, 97%, 98%, or 99% identical to SEQ ID NO: 93 or 98. In some embodiments, the fusion protein comprises or consists of SEQ ID NO: 93 or 98. In some embodiments, the effector protein comprises at least one amino acid alteration relative to the amino acid sequence of SEQ ID NO: 1. In some embodiments, the at least one amino acid alteration comprises an amino acid alteration set forth in TABLE 1.1. In some embodiments, the effector protein comprises at least one amino acid alteration relative to the amino acid sequence of SEQ ID NO: 2. In some embodiments, the at least one amino acid alteration comprises an amino acid alteration set forth in TABLE 1.2.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1 and 2 and an RDDP comprising an amino acid sequence that is at least 90%, 91%, 92%, 93%, 94%, 95%, 96%, 97%, 98%, or 99% identical to SEQ ID NO: 40. In some embodiments, the fusion protein described herein comprises an effector protein comprising or consisting of an amino acid sequence selected from SEQ ID NOs: 1 and 2 and an RDDP comprising or consisting of the amino acid sequence of SEQ ID NO: 40. In some embodiments, the fusion protein comprises an amino acid sequence that is at least 90%, 91%, 92%, 93%, 94%, 95%, 96%, 97%, 98%, or 99% identical to SEQ ID NO: 94 or 99. In some embodiments, the fusion protein comprises or consists of SEQ ID NO: 94 or 99. In some embodiments, the effector protein comprises at least one amino acid alteration relative to the amino acid sequence of SEQ ID NO: 1. In some embodiments, the at least one amino acid alteration comprises an amino acid alteration set forth in TABLE 1.1. In some embodiments, the effector protein comprises at least one amino acid alteration relative to the amino acid sequence of SEQ ID NO: 2. In some embodiments, the at least one amino acid alteration comprises an amino acid alteration set forth in TABLE 1.2.

In some embodiments, the fusion protein described herein comprises an amino acid sequence that is at least 70%, at least 75%, at least 80%, at least 85%, at least 90%, at least 95%, at least 97%, at least 98%, at least 99%, or 100% identical to an amino acid sequence selected from SEQ ID NOs: 1610-1619. In some embodiments, the fusion protein comprises an amino acid sequence that is at least 70%, at least 75%, at least 80%, at least 85%, at least 90%, at least 95%, at least 97%, at least 98%, at least 99%, or 100% identical to an amino acid sequence selected from SEQ ID NOs: 1611, 1613, 1615, 1618, and 1619, wherein the amino acid sequence comprises a P2A peptide that results in cleavage of the fusion protein at the site of the P2A.

### Tethers

In some embodiments the effector protein is complexed with all or part of a biological tether or a protein localization sequence. In some embodiments the RDDP is bound to the biological tether or protein localization sequence. In some embodiments the guide RNA is bound to an aptamer that is recognized by the biological tether protein.

In some embodiments, the biological tether or protein localization sequence is MS2, Csy4 or lambda N protein.

In some embodiments, the effector proteins are complexed with a biological tether, and comprises all or part of (e.g., DNA binding domain from) the MS2 coat protein, endoribonuclease Csy4, or the lambda N protein. These proteins can be used to recruit RNA molecules containing a specific stem-loop structure to a locale specified by the Type V effector protein guide RNA targeting sequences. For example, a Type V effector protein variant linked to MS2 coat protein, endoribonuclease Csy4, or lambda N can be used to recruit a long non-coding RNA (lncRNA) such as XIST or HOTAIR; see, e.g., Keryer-Bibens et al., Biol. Cell 100:125-138 (2008), that is linked to the Csy4, MS2 or lambda N binding sequence. Alternatively, the Csy4, MS2 or lambda N protein binding sequence can be linked to another protein, e.g., as described in Keryer-Bibens et al., supra, and the protein can be targeted to the dCpf1 variant binding site using the methods and compositions described herein. In some embodiments, the Csy4 is catalytically inactive.

In some embodiments, an RDDP is bound to an MCP (MS2 aptamer binding protein). In some embodiments, an MCP comprises an amino acid sequence that is at least 65%, at least 70%, at least 75%, at least 80%, at least 85%, at least 90%, at least 95%, at least 97%, at least 98%, at least 99%, or 100% identical to

(SEQ ID NO: 1626)
ASNFTQFVLVDNGGTGDVTVAPSNFANGVAEWISSNSRSQAYKVTCSVR
QSSAQKRKYTIKVEVPKVATQTVGGVELPVAAWRSYLNMELTIPIFATN
SDCELIVKAMQGLLKDGNPIPSAIAANSGIY.

In some embodiments, a fusion protein comprises a Brex27 peptide that inhibits non-homologous end joining (NHEJ). In some embodiments, a Brex27 peptide comprises an amino acid sequence that is at least 65%, at least 70%, at least 75%, at least 80%, at least 85%, at least 90%, at least 95%, at least 97%, at least 98%, at least 99%, or 100% identical to ALDFLSRLPLPPPVSPICTFVSPAAQKAFQPPRSCG (SEQ ID NO: 1625).

### Synthesis, Isolation and Assaying

RDDPs, effector proteins and fusion proteins of the present disclosure of the present disclosure may be synthesized, using any suitable method. Additionally, nucleic acids, including mRNA encoding effector proteins and fusion proteins of the present disclosure may be synthesized using suitable methods. RDDPs, effector proteins and fusion proteins of the present disclosure may be produced in vitro or by eukaryotic cells or by prokaryotic cells. Effector proteins can be further processed by unfolding, e.g. heat denaturation, dithiothreitol reduction, etc. and may be further refolded, using any suitable method.

Methods of generating and assaying the RDDPs, effector proteins and fusion proteins described herein are well known to one of skill in the art. Examples of such methods are described in the Examples provided herein. Any of a variety of methods can be used to generate an effector protein disclosed herein. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). One non-limiting example of a method for preparing an effector protein is to express recombinant nucleic acids encoding the effector protein in a suitable microbial organism, such as a bacterial cell, a yeast cell, or other suitable cell, using methods well known in the art.

In some embodiments, an RDDP, effector protein, and/or fusion protein provided herein is an isolated effector protein. In some embodiments, an RDDP, effector protein, and/or fusion protein described herein can be isolated and purified for use in compositions, systems, and/or methods described herein. Methods described here can include the step of isolating effector proteins described herein. An isolated an RDDP, effector protein, and/or fusion protein provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

RDDPs, effector proteins, and/or fusion proteins disclosed herein may be covalently linked or attached to a tag, e.g., a purification tag. A purification tag, as used herein, can be an amino acid sequence which can attach or bind with high affinity to a separation substrate and assist in isolating the protein of interest from its environment, which can be its biological source, such as a cell lysate. Attachment of the purification tag can be at the N or C terminus of the effector protein. Furthermore, an amino acid sequence recognized by a protease or a nucleic acid encoding for an amino acid sequence recognized by a protease, such as TEV protease or the HRV3C protease can be inserted between the purification tag and the effector protein, such that biochemical cleavage of the sequence with the protease after initial purification liberates the purification tag. Purification and/or isolation can be through high performance liquid chromatography (HPLC), exclusion chromatography, gel electrophoresis, affinity chromatography, or other purification technique. Examples of purification tags are as described herein.

### Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid.

In general, a guide nucleic acid is a nucleic acid molecule, at least a portion of which may be bound by an effector protein, thereby forming a ribonucleoprotein complex (RNP). The portion of the guide nucleic acid that may be bound by an effector protein may be referred to as the “protein binding sequence.” The protein binding sequence may comprise a repeat sequence, and intermediary sequence, a handle sequence, or a combination thereof, all of which are described herein and throughout. Another portion of the guide nucleic acid molecule can comprise a spacer region which is complementary to at least a portion of the target nucleic acid sequence. In some embodiments, the guide nucleic acid imparts activity or sequence selectivity to the effector protein. When complexed with an effector protein, guide nucleic acids can bring the effector protein into proximity of a target nucleic acid. The guide nucleic acid spacer region may hybridize to a target nucleic acid or a portion thereof. In some embodiments, when a guide nucleic acid and an effector protein form an RNP, at least a portion of the RNP binds spacer region, recognizes, and/or hybridizes to a target nucleic acid. Those skilled in the art in reading the below specific examples of guide nucleic acids as used in RNPs described herein, will understand that in some embodiments, a RNP can hybridize, via the spacer region, to one or more target sequences in a target nucleic acid, thereby allowing the RNP to modify and/or recognize a target nucleic acid or sequence contained therein.

A guide nucleic acid, as well as any components thereof (e.g., spacer region, repeat region, linker) may comprise one or more deoxyribonucleotides, ribonucleotides, biochemically or chemically modified nucleotides (e.g., one or more sequence modifications as described herein), and any combinations thereof. A guide nucleic acid may comprise a naturally occurring sequence. A guide nucleic acid may comprise a non-naturally occurring sequence, wherein the sequence of the guide nucleic acid, or any portion thereof, may be different from the sequence of a naturally occurring nucleic acid. The guide nucleic acid may be chemically synthesized or recombinantly produced. Guide nucleic acids and portions thereof may be found in or identified from a CRISPR array present in the genome of a host organism or cell.

Guide nucleic acids, while often being referred to as a guide RNA, may include deoxyribonucleosides, ribonucleosides, chemically modified nucleosides, or any combination thereof.

In some embodiments, the guide nucleic acid comprises a protein binding sequence that is bound by an effector protein. The protein binding sequence may also be referred to a scaffold. In some embodiments, the guide nucleic acid or scaffold thereof comprises a hairpin or stem-loop structure that is recognized by the effector protein. The hairpin or stem-loop structure may comprise a repeat region. In some embodiments, the guide nucleic acid comprises at least a portion of a tracrRNA sequence. In nature, a tracrRNA is a guide nucleic acid that has trans activating activity on a target nucleic acid through a repeat hybridization sequence. In some instances, guide nucleic acids of the instant disclosure do not comprise a repeat hybridization sequence. In some instances, guide nucleic acids of the instant disclosure do not comprise a tracrRNA. In some instances, e.g., systems comprising CasPhi proteins and fusions thereof, the guide nucleic acid does not comprise a tracrRNA sequence but comprises a repeat sequence to which the CasPhi protein binds.

In some embodiments, the guide nucleic acid comprises a repeat region that interacts with the effector protein. The term, “repeat region” may be used interchangeably herein with the term, “repeat sequence.” In some instances, an effector protein interacts with a repeat region. In some instances, an effector protein does not interact with a repeat region. Typically, the repeat region is adjacent to the spacer region. In certain embodiments, the repeat region is followed by the spacer region in the 5′ to 3′ direction. Exemplary repeat region sequences for exemplary effector proteins provided herein are shown in TABLE 8.

Exemplary guide RNA sequences to be used in combination with CasM.265466 or its engineered variants for targeting I) MI) are provided in TABLE 6. In some embodiments, a guide RNA comprises a nucleotide sequence that is at least 90%, at least 95%, at least 97%, at least 98%, at least 99%, or 100% identical to any one of the nucleotide sequences of SEQ ID NOs: 466-648 described in TABLE 6. In some embodiments, the guide RNA comprises a nucleotide sequence that is at least 90%, at least 95%, at least 97%, at least 98%, at least 99%, or 100% identical to any one of the nucleotide sequences of SEQ ID NOs: 649-831 described in TABLE 6, and SEQ ID NOs: 1620-1621.

Exemplary guide RNA sequences to be used in combination with CasPhi.12 or its engineered variants for targeting DMD are provided in TABLE 8. In some embodiments, a guide RNA comprises a nucleotide sequence that is at least 90%, at least 95%, at least 97%, at least 98%, at least 99%, or 100% identical to any one of the nucleotide sequences of SEQ ID NOs: 1212-1353 described in TABLE 8. In some embodiments, the guide RNA comprises a nucleotide sequence that is at least 90%, at least 95%, at least 97%, at least 98%, at least 99%, or 100% identical to any one of the nucleotide sequences of SEQ ID NOs: 1354-1495 described in TABLE 8.

### Spacer Sequences

In general, guide nucleic acids comprise a spacer sequence that hybridizes with a target sequence of a target nucleic acid. The term, “spacer sequence.” refers to a region of the guide nucleic acid that hybridizes to a target sequence of a target nucleic acid. The terms “spacer sequence” and “spacer region” are used interchangeably herein and throughout. The spacer sequence may comprise a sequence that is complementary with a target sequence of a target nucleic acid. In some embodiments, the spacer sequence is complementary to the target sequence on the target strand of a dsDNA molecule. In some embodiments, the spacer sequence is complementary to the target sequence on the non-target strand of a dsDNA molecule. The spacer sequence can function to direct the guide nucleic acid to the target nucleic acid for detection and/or modification of the target nucleic acid. The spacer sequence may be complementary to a target sequence that is adjacent to a PAM that is recognizable by an effector protein of interest.

In some embodiments, the spacer sequence is 15-28 linked nucleotides in length. In some embodiments, the spacer sequence is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer sequence is 18-24 linked nucleotides in length. In some embodiments, the spacer sequence is at least 15 linked nucleotides in length. In some embodiments, the spacer sequence is at least 16, 18, 20, or 22 linked nucleotides in length. In some embodiments, the spacer sequence comprises at least 10, 11, 12, 13, 14, 15, 16, 17, 18, 19, 20, 21, 22, 23, 24, 25, 26, 27, 28, 29, or 30 nucleotides. In some embodiments, the spacer sequence is at least 17 linked nucleotides in length. In some embodiments, the spacer sequence is at least 18 linked nucleotides in length. In some embodiments, the spacer region is at least 20 linked nucleotides in length. In some embodiments, the spacer sequence is at least 80%, at least 85%, at least 90%, at least 95% or 100% complementary to a target sequence of the target nucleic acid. In some embodiments, the spacer sequence is 100% complementary to the target sequence of the target nucleic acid. In some embodiments, the spacer sequence comprises at least 15 contiguous nucleotides that are complementary to the target nucleic acid. It is understood that the sequence of a spacer sequence need not be 100% complementary to that of a target sequence of a target nucleic acid to hybridize or hybridize specifically to the target sequence. The spacer sequence may comprise at least one nucleotide that is not complementary to the corresponding nucleotide of the target sequence. In some embodiments the spacer sequence is less than 100% complementary to the target sequence, but still can bind to the target sequence. In some embodiments the spacer sequence is 90%, 91%, 92%, 93%, 94%, 95%, 96%, 97%, 98%, 99%, or 100% complementary to the target sequence.

In some embodiments, a guide nucleic acid, a spacer region thereof or a spacer sequence thereof, comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a sequence of nucleotides that is present in a host eukaryotic cell. Such a sequence of nucleotides is distinguished from nucleotide sequences present in other host cells, such as prokaryotic cells, or viruses. By way of non-limiting example, said sequences present in a eukaryotic cell can be located in a gene, an exon, an intron, or a non-coding (e.g., promoter or enhancer) region. In some embodiments, a linker is present between the spacer and repeat sequences.

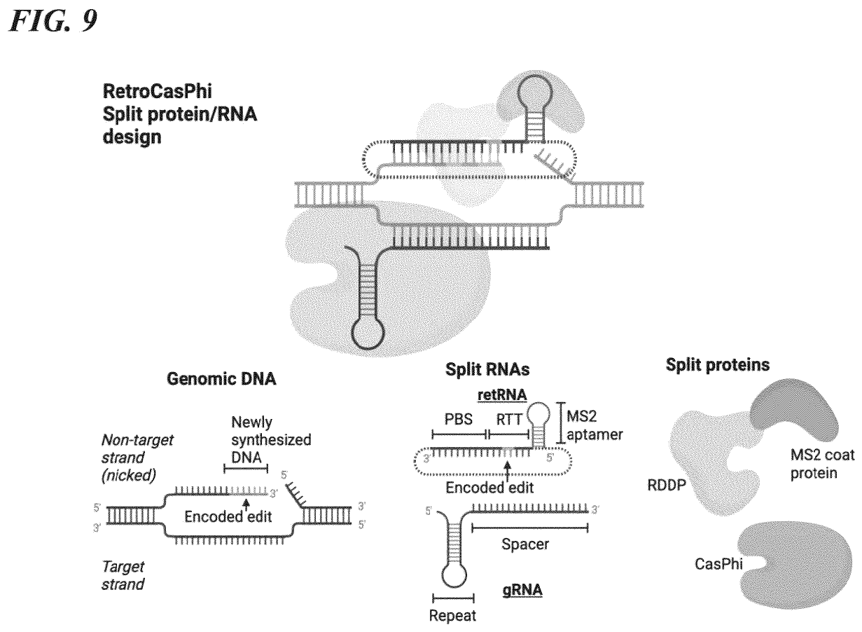

In some instances, a guide nucleic acid comprises a spacer length that confers nickase activity to a Cas enzyme that otherwise comprises nuclease activity when used with guide nucleic acids having a different spacer length. In some instances, the Cas enzyme is a Type V Cas enzyme. In some instances, the Cas enzyme is a CasPhi enzyme. In some cases, the Cas enzyme is CasPhi. 12 (SEQ ID NO: 2). In some cases, the spacer length that confers nickase activity is 14-16 nucleotides. A non-limiting example of such systems is provided in FIG. 9.

In some embodiments, the spacer sequence is at least partially complementary to an exon within the human dystrophin gene. In some embodiments, the spacer sequence is at least 80%, at least 85%, at least 90%, at least 95%, or 100% complementary to an equal length portion of an exon within the human dystrophin gene. In some embodiments, the spacer sequence is at least 80%, at least 85%, at least 90%, at least 95%, or 100% complementary to an equal length portion of a sequence within 10, 20, 30, 40, 50, 60, 70, 80, 90, or 100 nucleotides of an exon within the human dystrophin gene. In some embodiments, the exon is selected from exon 44, exon 45, exon 50, exon 51, exon 53, or any combination thereof, of the human dystrophin gene. In some embodiments, the spacer sequence is at least 80%, at least 85%, at least 90%, at least 95% or 100% identical to a spacer sequence described in TABLE 6 or TABLE 8.

Exemplary spacer sequences of guide RNAs to be used in combination with CasM.265466 or its engineered variants for targeting DMD are provided in TABLE 6. In some embodiments, the guide RNA comprises a spacer sequence that hybridizes to a target sequence in a target nucleic acid of the DMD gene and comprises a nucleotide sequence that is at least 90%, at least 95%, at least 97%, at least 98%, at least 99%, or 100% identical to any one of the nucleotide sequences of SEQ ID NOs: 100-282 described in TABLE 6.

Exemplary spacer sequences of guide RNAs to be used in combination with CasPhi.12 or its engineered variants for targeting DMD are provided in TABLE 8. In some embodiments, the guide RNA comprises a spacer sequence that hybridizes to a target sequence of DMD and comprises a nucleotide sequence that is at least 90%, at least 95%, at least 97%, at least 98%, at least 99%, or 100% identical to any one of the nucleotide sequences of SEQ ID NOs: 928-1069 described in TABLE 8.

### Nucleic Acid Linkers

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises two or more linkers. In some embodiments, at least two or more linkers are the same. In some embodiments, at least two or more linkers are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### Repeat Sequences

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence includes a nucleotide sequence that is capable of forming a guide nucleic acid-effector protein complex (e.g., a RNP complex). In some embodiments, the repeat sequence may also be referred to as a “protein-binding segment.”

In some embodiments, a repeat sequence is adjacent to a spacer sequence. In some embodiments, a repeat sequence is followed by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence, which may be a direct link or by any suitable linker, examples of which are described herein.

In some embodiments, the spacer sequence(s) and the repeat sequence(s) of the guide nucleic acid are present within the same polynucleotide molecule. In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer(s) and repeat sequence(s) are linked directly to one another. In some embodiments, a linker is present between the spacer(s) and repeat sequence(s). Linkers may be any suitable linker. In some embodiments, the spacer sequence(s) and the repeat sequence(s) of the guide nucleic acid are present in separate polynucleotide molecules, which are joined to one another by base pairing interactions.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

In some embodiments, the effector protein is at least 90%, at least 95%, at least 98%, at least 99% or 100% identical to SEQ ID NO: 2, and the repeat sequence is at least 80%, at least 90% or 100% identical to 5′-AUUGCUCCUUACGAGGAGAC-3′ (SEQ ID NO: 6).

Exemplary repeat sequences of guide RNAs to be used in combination with CasPhi.12 or its engineered variants for targeting DMD are provided in TABLE 8. In some embodiments, the repeat sequence comprises a nucleotide sequence that is at least 90%, at least 95%, at least 97%, at least 98%, at least 99%, or 100% identical to the nucleotide sequence of SEQ ID NO: 6 described in TABLE 8.

### Intermediary Sequence

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary sequence is at least 30, 50, 70, 90, 110, 130, 150, 170, 190, or 210 linked nucleotides. In some embodiments, a length of the intermediary sequence is not greater than 30, 50, 70, 90, 110, 130, 150, 170, 190, or 210 linked nucleotides. In some embodiments, the length of the intermediary sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

### Handle Sequence

In some embodiments, compositions, systems and methods described herein comprise the nucleic acid, wherein the nucleic acid comprises a handle sequence. In some embodiments, the handle sequence comprises an intermediary sequence. In some embodiments, the intermediary sequence is at the 3′-end of the handle sequence. In some embodiments, the intermediary sequence is at the 5′-end of the handle sequence. In some embodiments, the handle sequence further comprises one or more of linkers and repeat sequences. In some embodiments, the linker comprises a sequence of 5′-GAAA-3.′ In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker.

In some embodiments, a sgRNA may include a handle sequence having a hairpin region, as well as a linker and a repeat sequence. The sgRNA having a handle sequence can have a hairpin region positioned 3′ of the linker and/or repeat sequence. The sgRNA having a handle sequence can have a hairpin region positioned 5′ of the linker and/or repeat sequence. The hairpin region may include a first sequence, a second sequence that is reverse complementary to the first sequence, and a stem-loop linking the first sequence and the second sequence.

In some embodiments, an effector protein may recognize a secondary structure of a handle sequence. In some embodiments, at least a portion of the handle sequence interacts with an effector protein described herein. Accordingly, in some embodiments, at least a portion of the intermediary sequence interacts with the effector protein described herein. In some embodiments, both, at least a portion of the intermediary sequence and at least a portion of the repeat sequence, interacts with the effector protein. In general, the handle sequence is capable of interacting (e.g., non-covalent binding) with any one of the effector proteins described herein.

In some embodiments, the handle sequence of a sgRNA comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the sgRNA comprises a pseudoknot (e.g., a secondary structure comprising a stem at least partially hybridized to a second stem or half-stem secondary structure). An effector protein may recognize a sgRNA comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the sgRNA comprises at least 2, at least 3, at least 4, or at least 5 stem regions.

A handle sequence may include deoxyribonucleosides, ribonucleosides, chemically modified nucleosides, or any combination thereof. In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

In some embodiments, the length of a handle sequence in a sgRNA is not greater than 50, 56, 66, 67, 68, 69, 70, 71, 72, 73, 95, or 105 linked nucleotides. In some embodiments, the length of a handle sequence in a sgRNA is about 30 to about 120 linked nucleotides. In some embodiments, the length of a handle sequence in a sgRNA is about 50 to about 105, about 50 to about 95, about 50 to about 73, about 50 to about 71, about 50 to about 70, or about 50 to about 69 linked nucleotides. In some embodiments, the length of a handle sequence in a sgRNA is 56 to 105 linked nucleotides, from 56 to 105 linked nucleotides, 66 to 105 linked nucleotides, 67 to 105 linked nucleotides, 68 to 105 linked nucleotides, 69 to 105 linked nucleotides, 70 to 105 linked nucleotides, 71 to 105 linked nucleotides, 72 to 105 linked nucleotides, 73 to 105 linked nucleotides, or 95 to 105 linked nucleotides. In some embodiments, the length of a handle sequence in a sgRNA is 40 to 70 nucleotides. In some embodiments, the length of a handle sequence in a sgRNA is 50, 56, 66, 67, 68, 69, 70, 71, 72, 73, 95, or 105 linked nucleotides.

Exemplary handle sequences of guide RNAs to be used in combination with CasM.265466 or its engineered variants for targeting DMD are provided in TABLE 6. In some embodiments, the guide RNA comprises a handle sequence comprising a nucleotide sequence that is at least 90%, at least 95%, at least 97%, at least 98%, at least 99%, or 100% identical to the nucleotide sequence of SEQ ID NO: 283 described in TABLE 6.

### Single Nucleic Acid Systems

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR) of the guide nucleic acid non-covalently interacts with the one or more polypeptides described herein. In some embodiments, a second region (SR) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA.

crRNA

In some embodiments, guide nucleic acid comprises a crRNA comprising a spacer sequence(s) and a repeat sequence(s) present within the same polynucleotide molecule. In some embodiments, the spacer sequence is adjacent to the repeat sequence. In some embodiments, the spacer sequence follows the repeat sequence in a 5′ to 3′ direction. In some embodiments, the spacer sequence precedes the repeat sequence in a 5′ to 3′ direction. In some embodiments, the spacer(s) and repeat sequence(s) are linked directly to one another. In some embodiments, a linker is present between the spacer(s) and repeat sequence(s). Linkers may be any suitable linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an intermediary RNA. In some embodiments, a single nucleic acid system comprises a guide nucleic acid comprising a crRNA and an intermediary RNA.

In some embodiments, a crRNA is sufficient to form complex with an effector protein (e.g., to form an RNP) through the repeat sequence and direct the effector protein to a target nucleic acid sequence through the spacer sequence. In some embodiments, the repeat sequence in the crRNA polynucleotide hybridizes with a tracr sequence present in a separate polynucleotide. In some embodiments, the hybridization with the tracr sequences permits formation of an RNP complex with an effector protein.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

sgRNA

In some embodiments, a guide nucleic acid comprises a single guide RNA (sgRNA). In some embodiments, the guide nucleic acid is a sgRNA. The combination of a spacer sequence (e.g., a nucleotide sequence that hybridizes to a target sequence in a target nucleic acid) with a handle sequence may be referred to herein as a single guide RNA (sgRNA), wherein the spacer sequence and the handle sequence are covalently linked. In some embodiments, the spacer sequence and handle sequence are linked by a phosphodiester bond. In some embodiments, the spacer sequence and handle sequence are linked by one or more linked nucleotides. In some embodiments, a guide nucleic acid may comprise a spacer sequence, a repeat sequence, or handle sequence, or a combination thereof. In some embodiments, the handle sequence may comprise a portion of, or all of, a repeat sequence. In general, a sgRNA comprises a first region (FR) and a second region (SR), wherein the FR comprises a handle sequence and the SR comprises a spacer sequence.

In some embodiments, the compositions comprising a guide RNA and an effector protein without a tracrRNA (e.g., a single nucleic acid system), wherein the guide RNA is a sgRNA. A sgRNA may include deoxyribonucleosides, ribonucleosides, chemically modified nucleosides, or any combination thereof. A sgRNA may also include a nucleotide sequence that forms a secondary structure (e.g., one or more hairpin loops) that facilitates the binding of an effector protein to the sgRNA and/or modification activity of an effector protein on a target nucleic acid (e.g., a hairpin region). Such a sequence can be contained within a handle sequence as described herein.

In some embodiments, a sgRNA comprises one or more of one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and an crRNA; an intermediary sequence, a repeat sequence and a spacer sequence; and the like.

In some embodiments, a sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g., covalently linked, such as through a phosphodiester bond) In some embodiments, a repeat sequence and a spacer sequence are linked in an sgRNA by any suitable linker, examples of which are provided herein.

An exemplary handle sequence in a sgRNA may comprise, from 5′ to 3′, a 5′ region, a hairpin region, and a 3′ region. In some embodiments, the 5′ region may hybridize to the 3′ region. In some embodiments, the 5′ region does not hybridize to the 3′ region. In some embodiments, the 3′ region is covalently linked to a spacer sequence (e.g., through a phosphodiester bond). In some embodiments, the 5′ region is covalently linked to a spacer sequence (e.g., through a phosphodiester bond).

### Dual Nucleic Acid Systems

In some embodiments, compositions, systems and methods described herein comprise a dual nucleic acid system comprising a crRNA or a nucleotide sequence encoding the crRNA, a tracrRNA or a nucleotide sequence encoding the tracrRNA, and one or more effector proteins or a nucleotide sequence encoding the one or more effector proteins, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, in a dual nucleic acid system, activity of the effector protein requires binding to a tracrRNA molecule.

In some embodiments, a repeat hybridization sequence is at the 3′ end of a tracrRNA. In some embodiments, a repeat hybridization sequence may have a length of about 1, about 2, about 3, about 4, about 5, about 6, about 7, about 8, about 9, about 10, about 12, about 14, about 16, about 18, or about 20 linked nucleotides. In some embodiments, the length of the repeat hybridization sequence is 1 to 20 linked nucleotides.

In some embodiments, systems, compositions, and methods comprise a crRNA or a use thereof. In general, a crRNA comprises a first region (FR) and a second region (SR), wherein the FR of the crRNA comprises a repeat sequence, and the SR of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, systems, compositions, and methods comprise a tracrRNA or a use thereof. In some embodiments, systems, compositions, and methods do not comprise a tracrRNA or a use thereof. A tracrRNA and/or tracrRNA-crRNA duplex may form a secondary structure that facilitates the binding of an effector protein to a tracrRNA or a tracrRNA-crRNA. In some embodiments, the secondary structure modifies activity of the effector protein on a target nucleic acid. In some embodiments, the secondary structure comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the secondary structure comprises a pseudoknot (e.g., a secondary structure comprising a stem at least partially hybridized to a second stem or half-stem secondary structure). An effector protein may recognize a secondary structure comprising multiple stem regions. In some embodiments, nucleotide sequences of the multiple stem regions are identical to one another. In some embodiments, the nucleotide sequences of at least one of the multiple stem regions is not identical to those of the others. In some embodiments, the secondary structure comprises at least two, at least three, at least four, or at least five stem regions. In some embodiments, the secondary structure comprises one or more loops. In some embodiments, the secondary structure comprises at least one, at least two, at least three, at least four, or at least five loops.

### Guide Nucleic Acids for Precision Editing

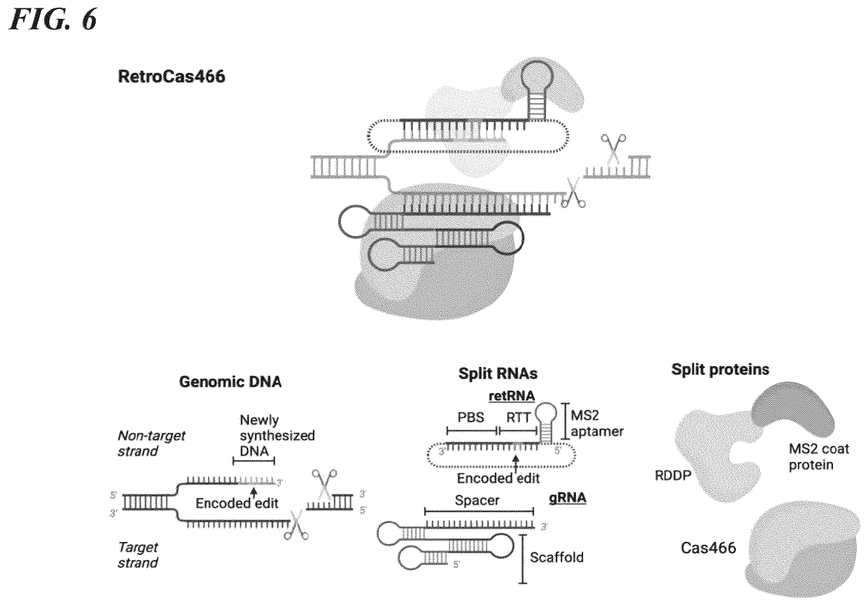

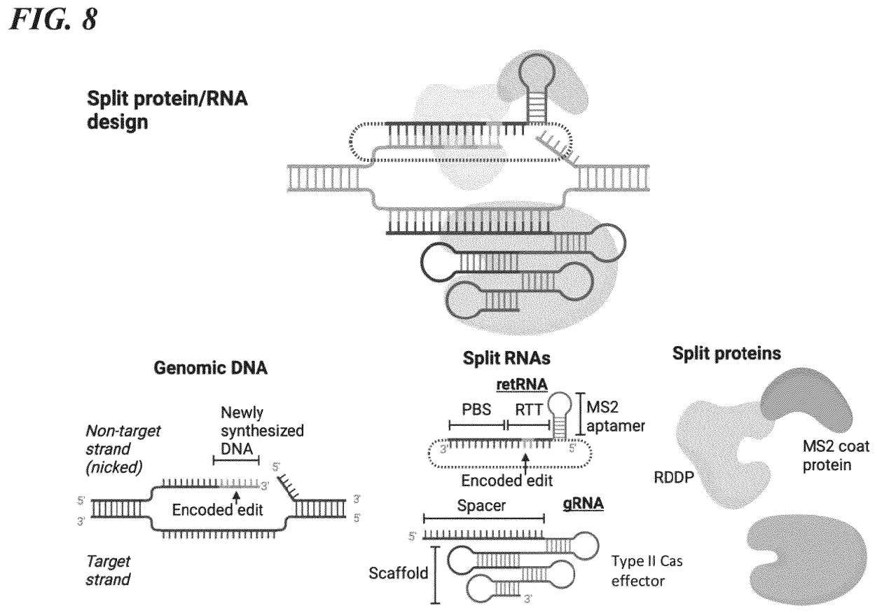

In some embodiments, the present disclosure provides guide nucleic acids for use in combination with the effector proteins, RDDPs, and fusion proteins thereof described herein for precision editing of a target nucleic acids sequence. In general, guide nucleic acids for use in precision editing comprise a spacer sequence, a repeat sequence, a primer binding sequence, and a template sequence. In some embodiments, guide nucleic acids for use in precision editing comprise one or more linkers between one or more components of the guide nucleic acids. In some embodiments, a spacer sequence, a repeat sequence, a primer binding sequence, and a template sequence are comprised in a single polynucleotide, referred to herein as an extended guide RNA (rtgRNA). See e.g., FIG. 3 and FIG. 4. In some embodiments, a spacer sequence, a repeat sequence, a primer binding sequence, and a template sequence are comprised in two polynucleotides—the spacer and repeat sequence comprised in a first polynucleotide and the primer binding sequence and the template sequence comprised in a second polynucleotide, referred to herein as a split RNA. See e.g., FIG. 5A, FIG. 5B, FIG. 6, FIG. 8 and FIG. 9.

### Template RNA

In some instances, compositions, systems and methods described herein comprise a template RNA, wherein the template RNA comprises a primer binding sequence and a template sequence. The template RNA may also be referred to as an extension of a guide RNA. The extension may comprise 1, 2, 3, 4, 5, 6, 7, 8, 9, 10, 11, 12, 13, 14, 15, 16, 17, 18, 19, or 20 nucleotides. The extension may comprise more than 10 nucleotides. In some embodiments the extension is 10-20 nucleotides. In some embodiments the extension is 20-30 nucleotides. In some embodiments the extension is 30-40 nucleotides. In some embodiments the extension is 40-50 nucleotides. In some embodiments the extension is 50-60 nucleotides. In some embodiments the extension is 70-80 nucleotides. In some embodiments the extension is 80-90 nucleotides. In some embodiments the extension is 90-100 nucleotides. In some embodiments the extension is 100-150 nucleotides. The extension may be processed during guide RNA formation. Template RNAs are also referred to herein retRNAs.

In some embodiments, the template RNA, the spacer sequence, and the repeat sequence are comprised in the same polynucleotide (e.g., an rtgRNA). In some embodiments, the spacer sequence and repeat sequence are comprised in a first polynucleotide and the template RNA is comprised in a second polynucleotide (e.g., a split RNA system).

In some instances, the primer binding sequence hybridizes to a primer sequence on the non-target strand of the target dsDNA molecule. In some instances, the primer binding sequence hybridizes to a primer sequence on the target strand of the target dsDNA molecule. In some embodiments, the spacer sequence is complementary to the target sequence on the target strand of the dsDNA molecule, and the primer binding sequence and/or the template sequence is complementary to a primer sequence on the non-target strand of the target dsDNA molecule. In some embodiments, the spacer sequence is complementary to the target sequence on the non-target strand of the dsDNA molecule, and the primer binding sequence and/or the template sequence is complementary to a primer sequence on the target strand of the target dsDNA molecule.

In some embodiments, the primer binding sequence is 4, 5, 6, 7, 8, 9, 10, 11, 12, 13, 14, 15, 16, 17, 18, 19, or 20 nucleotides long. In some embodiments the template sequence is at least 10, 11, 12, 13, 14, 15, 16, 17, 18, 19, 20, 21, 22, 23, 24, 25, 26, 27, 28, 29, 30, 31, 32, 33, 34 or 35 nucleotides long. In some embodiments, at least a portion of the PBS is complementary at least a portion of the target nucleic acid sequence that is 5′ of the nucleotide at position 13 relative to the PAM sequence. Such embodiments are particularly useful when used in a system comprising a CasM.265466 effector protein.

The template sequence may comprise one or more nucleotides having a different nucleobase than that of a nucleotide at the corresponding position in the target nucleic acid when a spacer sequence of the guide RNA and the target sequence are aligned for maximum identity. The one or more nucleotides may be contiguous. The one or more nucleotides may not be contiguous. The one or more nucleotides may each independently be selected from guanine, adenine, cytosine and thymine.

In some embodiments, the template RNA comprises a scaffold region. In general, the scaffold region is a constant region of the retRNA, remaining unchanged regardless of the specific target or edit. In some embodiments, the scaffold region comprises an MS2 hairpin. In some embodiments, the scaffold region comprises a ribozyme. In some embodiments, the scaffold region comprises an MS2 hairpin and a ribozyme. In such embodiments, the scaffold region enables the circularization of the retRNA to protect it from RNase degradation in cells. In some embodiments, the retRNA is expressed from a plasmid. In such embodiments, the retRNA comprises a guanine at the 5′ end, which is needed for transcription initiation from a U6 promoter. In general, the guanine doesn't serve a function on the retRNA itself.

Exemplary retRNA sequences to be used in combination with CasM.265466 or its engineered variants for targeting DMD are provided in TABLE 7 and SEQ ID NOs: 1622-1624. In some embodiments, a retRNA comprises a nucleotide sequence that is at least 90%, at least 95%, at least 97%, at least 98%, at least 99%, or 100% identical to any one of the nucleotide sequences of SEQ ID NOs: 880-903 described in TABLE 7. In some embodiments, the retRNA comprises a PBS comprising a nucleotide sequence that is at least 90%, at least 95%, at least 97%, at least 98%, at least 99%, or 100% identical to any one of the nucleotide sequences of SEQ ID NOs: 832-855 described in TABLE 7. In some embodiments, the retRNA comprises a template sequence (RTT) comprising a nucleotide sequence that is at least 90%, at least 95%, at least 97%, at least 98%, at least 99%, or 100% identical to any one of the nucleotide sequences of SEQ ID NOs: 856-879 described in TABLE 7. In some embodiments, the retRNA comprises a nucleotide sequence that is at least 90%, at least 95%, at least 97%, at least 98%, at least 99%, or 100% identical to any one of the nucleotide sequences of SEQ ID NOs: 904-927 described in TABLE 7 and SEQ ID NOs: 1622-1624.

Exemplary retRNA sequences to be used in combination with CasPhi. 12 or its engineered variants for targeting D) MI) are provided in TABLE 9. In some embodiments, a retRNA comprises a nucleotide sequence that is at least 90%, at least 95%, at least 97%, at least 98%, at least 99%, or 100% identical to any one of the nucleotide sequences of SEQ ID NOs: 1544-1567 described in TABLE 9. In some embodiments, the retRNA comprises a PBS comprising a nucleotide sequence that is at least 90%, at least 95%, at least 97%, at least 98%, at least 99%, or 100% identical to any one of the nucleotide sequences of SEQ ID NOs: 1496-1519 described in TABLE 9. In some embodiments, the retRNA comprises a template sequence (RTT) comprising a nucleotide sequence that is at least 90%, at least 95%, at least 97%, at least 98%, at least 99%, or 100% identical to any one of the nucleotide sequences of SEQ ID NOs: 1520-1543 described in TABLE 9. In some embodiments, the retRNA comprises a nucleotide sequence that is at least 90%, at least 95%, at least 97%, at least 98%, at least 99%, or 100% identical to any one of the nucleotide sequences of SEQ ID NOs: 1568-1591 described in TABLE 9.

Extended Guide RNA (rtgRNA) Systems

In some embodiments, the present disclosure provides extended guide nucleic acids (rtgRNA) and an effector protein and RDDP, or fusion protein thereof described herein, or nucleic acids encoding the same, wherein the spacer sequence, repeat sequence, template sequence, and primer binding sequence are each comprised in a single polynucleotide.

In some embodiments, the rtgRNA comprises, from 5′ to 3′, a template sequence, a primer binding sequence, a repeat sequence, and a spacer sequence, optionally wherein a linker sequence is located between the primer binding sequence and the repeat sequence. See e.g., FIG. 3. In some embodiments, the rtgRNA comprises, from 5′ to 3′, a repeat sequence, a spacer sequence, a template sequence, and a primer binding sequence, optionally wherein a linker sequence is located between the template sequence and the spacer sequence. See e.g., FIG. 4.

Split gRNA Systems

In some embodiments, the present disclosure provides a split gRNA system, comprising a first polynucleotide comprising a spacer sequence and a repeat sequence (e.g., a gRNA) and a second polynucleotide comprising a primer binding sequence and a template sequence (e.g., retRNA), and an effector protein and RDDP, or fusion protein thereof described herein, or nucleic acids encoding the same.

In some embodiments, the first polynucleotide comprises a spacer sequence and a repeat sequence. In some embodiments, the first polynucleotide is a crRNA, as described above. In some embodiments, the first polynucleotide comprises a spacer sequence and a handle sequence (also referred to herein as a scaffold sequence). In some embodiments, the first polynucleotide is an sgRNA, as described above.

In some embodiments, the second polynucleotide comprises a primer binding sequence and a template sequence (e.g., retRNA). In some embodiments, the second polynucleotide further comprises an aptamer that is recognized by a biological tether protein linked to an RDDP described herein. In some embodiments, the aptamer comprises a secondary structure (e.g., loops, stems, or hairpins). In some embodiments, the aptamer is located at one end of the second polynucleotide. In some embodiments, the same aptamer is located at both ends of the second polynucleotide. In some embodiments, different aptamers are located at each end of the second polynucleotide. In some embodiments, the aptamer is an MS2 aptamer (See Said et al (November 2009). “In vivo expression and purification of aptamer-tagged small RNA regulators”. Nucleic Acids Research. 37(20):e133; and Johansson et al (1997). “RNA recognition by the MS2 phage coat protein”. Seminars in Virology. 8(3):176-185). In some embodiments, the second polynucleotide comprises, from 5′ to 3′, an aptamer sequence, a template sequence, and a primer binding sequence. See FIG. 5B. In some embodiments, the second polynucleotide comprises, from 5′ to 3′, template sequence, a primer binding sequence, and an aptamer sequence. See FIG. 5A.

In some embodiments, the second polynucleotide is circularized. See FIG. 6.

Exemplary first and second polynucleotide sequences to be used in combination with CasM.265466 or its engineered variants for targeting IMI) are provided in TABLE 6 and TABLE 7. In some embodiments, the first polynucleotide comprises a nucleotide sequence that is at least 90%, at least 95%, at least 97%, at least 98%, at least 99%, or 100% identical to any one of the nucleotide sequences of SEQ ID NOs: 466-648 and 649-831. In some embodiments, the second polynucleotide comprises a nucleotide sequence that is at least 90%, at least 95%, at least 97%, at least 98%, at least 99%, or 100% identical to any one of the nucleotide sequences of SEQ ID NOs: 880-903 and 904-927.

Exemplary first and second polynucleotide sequences to be used in combination with CasPhi.12 or its engineered variants for targeting D) MI) are provided in TABLE 8 and TABLE 9. In some embodiments, the first polynucleotide comprises a nucleotide sequence that is at least 90%, at least 95%, at least 97%, at least 98%, at least 99%, or 100% identical to any one of the nucleotide sequences of SEQ ID NOs: 1212-1353 and 1354-1495. In some embodiments, the second polynucleotide comprises a nucleotide sequence that is at least 90%, at least 95%, at least 97%, at least 98%, at least 99%, or 100% identical to any one of the nucleotide sequences of SEQ ID NOs: 1544-1567 and 1568-1591.

### Integrases

In some embodiments, guide nucleic acids comprise an integration sequence. In some embodiments, the retRNA comprises an integration sequence. In some embodiments, the RTT sequence of the retRNA comprises an integration sequence. In some instances, the integration sequence is linked to a primer binding sequence. The guide nucleic acid may interact with the effector protein and target the effector protein to the desired location in the cell genome. The effector protein may nick a strand of the cell genome and the RDDP may incorporate the integration sequence of the guide nucleic acid into the nicked site. This provides an integration site at the desired location of the cell genome. In some embodiments, compositions and systems further comprise a donor nucleic acid comprising a sequence that is complementary to the integration site, and an integration enzyme, wherein the integration enzyme incorporates the donor nucleic acid into the cell genome at the integration site by integration, recombination, or reverse transcription of the sequence that is complementary to the integration site.

The integration enzyme can be a recombinase that incorporates the genome or nucleic acid of interest into the cell genome at the integration site by recombination. The integration enzyme can be an RNA-directed DNA polymerase, which in some embodiments can be a reverse transcriptase that incorporates the genome or nucleic acid of interest into the cell genome at the integration site by reverse transcription. The integration enzyme can be a retrotransposase that incorporates the genome or nucleic acid of interest into the cell genome at the integration site by retrotransposition.

In some embodiments, the integration enzyme is selected from the group consisting of Hin, Gin, Tn3, β-six, CinH, ParA, γδ, TP901, φBTI, φRVI, φFCI, Al18, U153, gp29, FLP, R, Lambda, HK101, HK022, and pSAM2, Cre, Dre, Vika, Bxbl, φC31, RDF, FLP, φBTI, R1, R2, R3, R4, R5, TP901-1, A118, φCl, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, LI, Tol2 Tc1, Tc3, Mariner (Himar 1), Mariner (mos 1), and Minos, and any mutants thereof. In some embodiments the integration enzyme is listed in WO2022087235, which is incorporated herein.

In some embodiments, the integration site can be selected from an attB site, an attP site, an attL site, an attR site, a lox71 site a Vox site, a Bxbl site, or a FRT site.

In some embodiments multiple genes are modified. In some embodiments the multiplexing is done by using multiple different integration sites with multiple different guide and effector proteins.

### Exemplary Systems

In some embodiments, the present disclosure provides systems for precision editing of a target nucleic acids sequence.

In some embodiments, provided herein are systems comprising: a) an effector protein; b) an RNA-directed DNA polymerase (RDDP); c) a guide RNA, wherein the guide RNA comprises a first region comprising a protein binding sequence, and a second region comprising a spacer sequence that hybridizes to a target sequence of a first strand of a double stranded DNA (dsDNA) target nucleic acid, wherein the dsDNA target nucleic acid comprises DMD, wherein the first region is located 5′ of the second region; and d) a template RNA (retRNA), wherein the retRNA comprises a primer binding sequence (PBS), and a template sequence (RTT) that hybridizes to at least a portion of the target sequence of a second strand of the dsDNA target nucleic acid. In some embodiments, the effector protein comprises four amino acid substitutions relative to SEQ ID NO: 2, wherein the amino acid substitutions comprise L26R, 1471T, S223P, and D703G. In some embodiments, the effector protein comprises an amino acid sequence that is at least 90%, at least 95%, at least 97%, at least 98%, at least 99%, or 100% identical to the amino acid sequence of SEQ ID NO: 1607. In some embodiments, the RDDP is not linked to the effector protein. In some embodiments, the RDDP is linked to an MS2 coat protein (MCP). In some embodiments, the RDDP comprises an amino acid sequence that is at least 90%, at least 95%, at least 97%, at least 98%, at least 99%, or 100% identical to the amino acid sequence of SEQ ID NO: 1608. In some embodiments, the guide RNA comprise a nucleotide sequence that is at least 90%, at least 95%, at least 97%, at least 98%, at least 99%, or 100% identical to the nucleotide sequence of SEQ ID NO: 1327 as described in TABLE 8. In some embodiments, the protein binding sequence comprises a nucleotide sequence that is at least 90%, at least 95%, at least 97%, at least 98%, at least 99%, or 100% identical to the nucleotide sequence of SEQ ID NO: 6. In some embodiments, the spacer sequence comprises a nucleotide sequence that is at least 90%, at least 95%, at least 97%, at least 98%, at least 99%, or 100% identical to the nucleotide sequence of SEQ ID NO: 1043. In some embodiments, the retRNA comprises a nucleotide sequence that is at least 90%, at least 95%, at least 97%, at least 98%, at least 99%, or 100% identical to the nucleotide sequence of SEQ ID NO: 1590 as described in TABLE 9. In some embodiments, the PBS comprises a nucleotide sequence that is at least 90%, at least 95%, at least 97%, at least 98%, at least 99%, or 100% identical to the nucleotide sequence of SEQ ID NO: 1518. In some embodiments, the RTT comprises a nucleotide sequence that is at least 90%, at least 95%, at least 97%, at least 98%, at least 99%, or 100% identical to the nucleotide sequence of SEQ ID NO: 1542.

### Target Nucleic Acids and Samples

Described herein are compositions, systems and methods for modifying a target sequence of the human dystrophin gene (D) MI)). In general, DMD comprises a target strand and a non-target strand, and at least a portion of the guide nucleic acids described herein is complementary to the target sequence on the target strand. In some embodiments, the target sequence is adjacent to a PAM as described herein that is located on the non-target strand. Such a PAM described herein, in some embodiments, is adjacent (e.g., within 1, 2, 3, 4, 5, 10, 20, 25 nucleotides) to the 5′ end of the target sequence on the non-target strand of the double stranded DNA molecule. In certain embodiments, such a PAM described herein is directly adjacent to the 5′ end of a target sequence on the non-target strand of the double stranded DNA molecule. In some embodiments, the PAM comprises a PAM sequence set forth in TABLE 1, TABLE 6 and TABLE 8.

In some embodiments, the target sequence is within an exon of the human dystrophin gene. In some embodiments, then target sequence covers the junction of two exons. In some embodiments, the target sequence is located within about 1 to about 300 nucleotides, about 10 to about 250, about 20 to about 200, about 30 to about 150, about 40 to about 100, or about 50 nucleotides of the 5′ untranslated region (UTR). In some embodiments, the target sequence is located within about 1 to about 300 nucleotides, about 10 to about 250, about 20 to about 200, about 30 to about 150, about 40 to about 100, or about 50 nucleotides of the 3′ UTR.

In some embodiments, the target sequence is at least partially within a targeted exon within the human dystrophin gene. As used herein the term “targeted exon” can mean any portion within, contiguous with, or adjacent to a specified exon of interest can be targeted by the compositions, systems, and methods described herein. In some embodiments, one or more of exons 1 to exon 79, exon 15 to exon 60, exon 20 to exon 55, exon 40 to exon 55, or exon 44 to exon 53 are targeted. In some embodiments, one or more of exon 44, exon 45, exon 50, exon 51, exon 52, exon 53, or any combination thereof, of the human dystrophin gene are targeted. Accordingly, in some embodiments; exon 44 is targeted; exon 45 is targeted; exon 50 is targeted; exon 51 is targeted; exon 52 is targeted; exon 53 is targeted; or any combination thereof. In some embodiments, certain exemplary genomic exons can be found in TABLE 6 and TABLE 8.

### Mutations

In some embodiments, target nucleic acids comprise a mutation. In some embodiments, a composition, system or method described herein can be used to modify a target nucleic acid comprising a mutation such that the mutation is modified to be a wild-type nucleotide or nucleotide sequence. In some embodiments, a composition, system or method described herein can be used to detect a target nucleic acid comprising a mutation. In some embodiments, a sequence comprising a mutation may be modified to a wild-type sequence with a composition, system or method described herein.

A mutation may be in an open reading frame of a target nucleic acid. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

In some embodiments, a mutation comprises a point mutation or single nucleotide polymorphism (SNP), a chromosomal mutation, a copy number mutation, or any combination thereof. A point mutation optionally comprises a substitution, insertion, or deletion.

In some embodiments, target nucleic acids comprise a mutation, wherein the mutation is a SNP. The single nucleotide mutation or SNP may be associated with a phenotype of the sample or a phenotype of the organism from which the sample was taken. The SNP, in some embodiments, is associated with altered phenotype from wild type phenotype. In some embodiments, a single nucleotide mutation, SNP, or deletion described herein is associated with a disease, such as a genetic disease. The SNP may be a synonymous substitution or a nonsynonymous substitution. The nonsynonymous substitution may be a missense substitution or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a cancer cell.

In some embodiments, the target nucleic acid comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

In some embodiments, a target nucleic acid described herein comprises a mutation associated with a disease, wherein the target nucleic acid is DMD (also known as: BMD, CMD3B, MRX85, DXS142, DXS164, DXS206, DXS230, DXS239, DXS268, DXS269, DXS270, DXS272). In some embodiments, the disease, disorder, condition, syndrome or pathological state comprises any one of the diseases, disorders, or pathological states as set forth in TABLE 13.

The mutation may cause a disease. The disease may comprise, at least in part, an inherited disorder, a neurological disorder, or both. The disease may comprise, at least in part, an inherited disorder. The disease may comprise, at least in part, a neurological disorder. In some embodiments, the neurological disorder to a neuromuscular disorder. In some embodiments, the neuromuscular disorder comprises: muscular dystrophy; duchenne muscular dystrophy (DMD); muscular dystrophy, pseudohypertrophic progressive, duchenne type; severe dystrophinopathy, duchenne type; muscular dystrophy duchenne type; becker muscular dystrophy (BMD); muscular dystrophy, pseudohypertrophic progressive, becker type; benign congenital myopathy; benign pseudohypertrophic muscular dystrophy; becker dystrophinopathy; muscular dystrophy pseudohypertrophic progressive, becker type; muscular dystrophy becker type; cardiomyopathy: x-linked dilated cardiomyopathy, type 3B (CMD3B); or dystrophinopathies.

### Methods of Treating a Disorder

Compositions, systems and methods disclosed herein may be useful for treating a disease in a subject by modifying a target nucleic acid associated with a gene or expression of a gene related to the disease. In some embodiments, methods comprise administering a composition or cell described herein to a subject. Exemplary diseases and syndromes include, but are not limited to, the diseases and syndromes listed in TABLE 13.

In some embodiments, methods of treating a disease in a subject comprise administering an RDDP and an effector protein described herein, or fusion protein comprising the same, a guide RNA, and a retRNA or rtgRNA described herein to the subject. In some embodiments, methods of treating a disease in a subject comprise administering one or more polynucleotides encoding one or more components of a system described herein, or one or more vectors comprising the same, e.g., a polynucleotide encoding an RDDP described herein (or a fusion protein comprising the same) or vector comprising the same, a polynucleotide encoding an effector protein described herein (or a fusion protein comprising the same) or a vector comprising the same, and/or a polynucleotide encoding an rtgRNA or a vector comprising the same.

### Donor Nucleic Acids

In some embodiments, a donor nucleic acid comprises a nucleic acid that is incorporated into a target nucleic acid or target sequence. In reference to a viral vector, the term donor nucleic acid refers to a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome via an integration sequence and an integrase. As another example, when used in reference to the activity of an effector protein, the term donor nucleic acid refers to a sequence of nucleotides that will be, or has been, inserted at the site of cleavage by the effector protein (cleaving (hydrolysis of a phosphodiester bond) of a nucleic acid resulting in a nick or double strand break-nuclease activity). As yet another example, when used in reference to homologous recombination, the term donor nucleic acid refers to a sequence of DNA that serves as a template in the process of homologous recombination, which may carry the modification that is to be or has been introduced into the target nucleic acid. By using this donor nucleic acid as a template, the genetic information, including the modification, is copied into the target nucleic acid by way of homologous recombination.

Donor nucleic acids of any suitable size may be integrated into a target nucleic acid or genome. In some embodiments, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some embodiments, donor nucleic acids are more than 500 kilobases (kb) in length.

### Chemical Modifications

Polypeptides (e.g., effector proteins) and nucleic acids (e.g., engineered guide nucleic acids) described herein can be further modified as described throughout and as further described herein. Examples are modifications of interest that do not alter primary sequence, including chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Modifications disclosed herein can also include modification of described polypeptides and/or engineered guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g. D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or engineered guide nucleic acids described herein. For example, groups can be introduced during synthesis or during expression of a polypeptide (e.g., an effector protein), which allow for linking to other molecules or to a surface. Thus, e.g., cysteines can be used to make thioethers, histidines for linking to a metal ion complex, carboxyl groups for forming amides or esters, amino groups for forming amides, and the like.

Modifications can further include modification of nucleic acids described herein (e.g., engineered guide nucleic acids) to provide the nucleic acid with a new or enhanced feature, such as improved stability. Such modifications of a nucleic acid include a base modification, a backbone modification, a sugar modification, or combinations thereof, of one or more nucleotides, nucleosides, or nucleobases in a nucleic acid.

In some embodiments, nucleic acids (e.g., engineered guide nucleic acids) described herein comprise one or more modifications comprising: 2′O-methyl modified nucleotides, 2′ Fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH2-NH—O—CH2-, —CH2-N(CH3)-O—CH2- (known as a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH2 component parts; and combinations thereof.

### Vectors and Multiplexed Expression Vectors

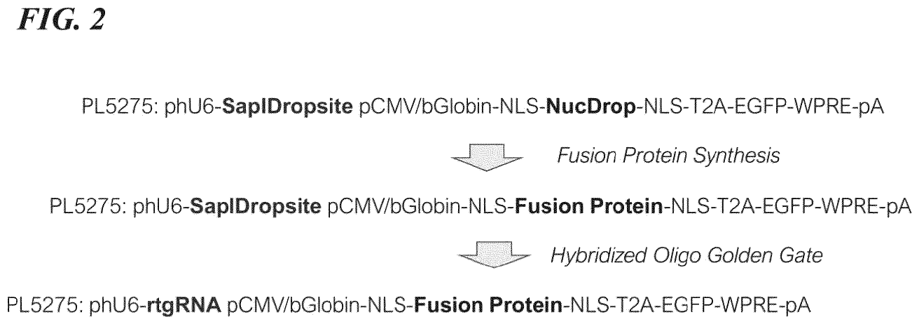

In some embodiments, compositions and systems provided herein comprise a vector system encoding a polypeptide (e.g., an effector protein, an RDDP, and/or a fusion protein thereof) and/or a guide nucleic acid described herein. In some embodiments, compositions and systems provided herein comprise a vector system encoding a guide nucleic acid (e.g., crRNA) described herein. In some embodiments, compositions and systems provided herein comprise a multi-vector system encoding an effector protein, an RDDP, and/or a fusion protein thereof and a guide nucleic acid described herein, wherein the guide nucleic acid and the effector protein, the RDDP, and/or the fusion protein thereof are encoded by the same or different vectors. In some embodiments, the guide and the engineered effector protein are encoded by different vectors of the system. In some embodiments, a nucleic acid encoding a polypeptide (e.g., an effector protein, an RDDP, and/or a fusion protein thereof) comprises an expression vector. In some embodiments, an expression vector encoding a fusion protein comprises a nucleic acid sequence encoding a P2A peptide between the sequences encoding an effector protein and an RDDP. In some embodiments, an expression vector encoding a fusion protein does not comprise a nucleic acid sequence encoding a P2A peptide between the sequences encoding an effector protein and an RDDP. In such embodiments, the RDDP is fused to the effector protein. The RDDP may not be fused to an MS2 coat protein and the template RNA may not comprise an MS2 protein localization sequence. In some embodiments, a nucleic acid encoding a polypeptide is a messenger RNA. In some embodiments, an expression vector comprises or encodes an engineered guide nucleic acid. In some embodiments, the expression vector encodes the crRNA. A non-limiting example of a vector design, and vector components which may be oriented in alternative manners, is depicted in FIG. 2.

In some embodiments, a vector can comprise or encode one or more regulatory elements. Regulatory elements can refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector can comprise or encode for one or more additional elements, such as, for example, replication origins, antibiotic resistance (or a nucleic acid encoding the same), a tag (or a nucleic acid encoding the same), selectable markers, and the like.

Vectors described herein can encode a promoter—a regulatory region on a nucleic acid, such as a DNA sequence, capable of initiating transcription of a downstream (3′ direction) coding or non-coding sequence. As used herein, a promoter can be bound at its 3′ terminus to a nucleic acid the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. A promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors can be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (Hl). By transcriptional activation, it is intended that transcription will be increased above basal levels in the target cell by 10 fold, by 100 fold, or by 1000 fold, or more. In addition, vectors used for providing a nucleic acid that, when transcribed, produces a guide nucleic acid and/or a nucleic acid that encodes an effector protein to a cell may include nucleic acid sequences that encode for selectable markers in the target cells, so as to identify cells that have taken up the guide nucleic acid and/or an effector protein.

In general, vectors provided herein comprise at least one promotor or a combination of promoters driving expression or transcription of one or more genome editing tools described herein. In some embodiments, the viral vector comprises a nucleotide sequence of a promoter. In some embodiments, the viral vector comprises two promoters. In some embodiments, the viral vector comprises three promoters. In some embodiments, the length of the promoter is less than about 500, less than about 400, or less than about 300 linked nucleotides. In some embodiments, the length of the promoter is at least 100 linked nucleotides. Non-limiting examples of promoters include CMV, 7SK, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, MSCV, MND and CAG.

In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, an effector protein (or a nucleic acid encoding same) and/or a guide nucleic acid (or a nucleic acid that, when transcribed, produces same) are co-administered with a donor nucleic acid. Coadministration can be contact with a target nucleic acid, administered to a cell, such as a host cell, or administered as method of nucleic acid detection, editing, and/or treatment as described herein, in a single vehicle, such as a single expression vector. In certain embodiments, an effector protein (or a nucleic acid encoding same) and/or a guide nucleic acid (or a nucleic acid that, when transcribed, produces same) are not co-administered with donor nucleic acid in a single vehicle. In certain embodiments, an effector protein (or a nucleic acid encoding same), a guide nucleic acid (or a nucleic acid that, when transcribed, produces same), and/or donor nucleic acid are administered in one or more or two or more vehicles, such as one or more, or two or more expression vectors.

### Viral Vectors

An expression vector can be a viral vector. In some embodiments, a viral vector comprises a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. In some embodiments, the expression vector is an adeno-associated viral vector. There are a variety of viral vectors that are associated with various types of viruses, including but not limited to retroviruses (e.g., lentiviruses and y-retroviruses), adenoviruses, arenaviruses, alphaviruses, adeno-associated viruses (AAVs), baculoviruses, vaccinia viruses, herpes simplex viruses and poxviruses. A viral vector provided herein can be derived from or based on any such virus. Often the viral vectors provided herein are an adeno-associated viral vector (AAV vector). Generally, an AAV vector has two inverted terminal repeats (ITRs). According, in some embodiments, the viral vector provided herein comprises two inverted terminal repeats of AAV. The DNA sequence in between the ITRs of an AAV vector provided herein may be referred to herein as the sequence encoding the genome editing tools or a transgene. These genome editing tools can include, but are not limited to, an effector protein, effector protein modifications (e.g., nuclear localization signal (NLS), polyA tail), guide nucleic acid(s), respective promoter(s), and a donor nucleic acid, or combinations thereof. In some embodiments, a nuclear localization signal comprises an entity (e.g., peptide) that facilitates localization of a nucleic acid, protein, or small molecule to the nucleus, when present in a cell that contains a nuclear compartment.

In general, viral vectors provided herein comprise at least one promotor or a combination of promoters driving expression or transcription of one or more genome editing tools described herein. In some embodiments, the length of the promoter is less than about 500, less than about 400, or less than about 300 linked nucleotides. In some embodiments, the length of the promoter is at least 100 linked nucleotides. Non-limiting examples of promoters include CMV, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, and MSCV. In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44.

In some embodiments, the coding region of the AAV vector forms an intramolecular double-stranded DNA template thereby generating an AAV vector that is a self-complementary AAV (scAAV) vector. In general, the sequence encoding the genome editing tools of an scAAV vector has a length of about 2 kb to about 3 kb. The scAAV vector can comprise nucleotide sequences encoding an effector protein, providing guide nucleic acids described herein, and a donor nucleic acid described herein. In some embodiments, the AAV vector provided herein is a self-inactivating AAV vector.

In some embodiments, an AAV vector provided herein comprises a modification, such as an insertion, deletion, chemical alteration, or synthetic modification, relative to a wild-type AAV vector.

In some embodiments, the viral particle that delivers the viral vector described herein is an AAV. AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof.

### Producing AAV Particles

The AAV particles described herein can be referred to as recombinant AAV (rAAV). Often, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as EIA, EIB, E2A, E4ORF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

### Lipid Particles

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector. In some embodiments, a lipid or a lipid nanoparticle can encapsulate the effector protein, the sgRNA or crRNA, the nucleic acid encoding the effector protein and/or the DNA molecule encoding the sgRNA or crRNA. LNPs are effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28 (3): 146-157). In some embodiments, a method can comprise contacting a cell with an expression vector. In some embodiments, contacting can comprise electroporation, lipofection, or lipid nanoparticle (LNP) delivery of an expression vector.

Methods and Formulations for Introducing Systems and Compositions into a Target Cell

A guide nucleic acid (or a nucleic acid comprising a nucleotide sequence encoding same) and/or an effector protein described herein can be introduced into a host cell by any of a variety of well-known methods. As a non-limiting example, a guide nucleic acid and/or effector protein can be combined with a lipid. As another non-limiting example, a guide nucleic acid and/or effector protein can be combined with a particle or formulated into a particle.

### Methods of Editing

Methods of editing described herein may be employed to generate a genetically modified cell. The cell may be a eukaryotic cell (e.g., a mammalian cell) or a prokaryotic cell (e.g., an archaeal cell). The cell may be derived from a multicellular organism and cultured as a unicellular entity. The cell may comprise a heritable genetic modification, such that progeny cells derived therefrom comprise the heritable genetic mutation. The cell may be progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

Methods may comprise contacting a cell or a subject with a fusion protein, effector protein, or partner protein disclosed herein, or any combination thereof. Methods may comprise contacting a cell with a nucleic acid encoding the fusion protein, effector protein, or partner protein, or combination thereof. The nucleic acid may be an expression vector. The nucleic acid may comprise a messenger RNA. Methods may comprise contacting a cell or a subject with an extended guide RNA or a portion thereof. Methods may comprise contacting a cell or a subject with a nucleic acid (e.g., DNA) encoding the extended guide RNA, or a portion thereof.

In some embodiments, the present disclosure provides a mammalian cell comprising a system described herein, e.g., an RDDP described herein (or a fusion protein comprising the same), an effector protein described herein (or a fusion protein comprising the same), and/or an rtgRNA. In some embodiments, the present disclosure provides a mammalian cell comprising one or more polynucleotides encoding one or more components of a system described herein, or one or more vectors comprising the same, e.g., a polynucleotide encoding an RDDP described herein (or a fusion protein comprising the same) or vector comprising the same, a polynucleotide encoding an effector protein described herein (or a fusion protein comprising the same) or a vector comprising the same, and/or a polynucleotide encoding an rtgRNA or a vector comprising the same.

Methods of the disclosure may be performed in a subject. Compositions of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be a patient. A subject may be at risk of developing, suffering from, or displaying symptoms a disease or disorder as set forth in herein. The subject may have a mutation associated with a gene described herein. The subject may display symptoms associated with a mutation of a gene described herein. In some embodiments, a mutation comprises a point mutation or single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutation can comprise an inversion, a deletion, a duplication, or a translocation. In some embodiments, a mutation comprises a copy number variation. A copy number variation can comprise a gene amplification or an expanding trinucleotide repeat. In some embodiments, mutations may be as described herein.

Methods of the disclosure may be performed in a cell. A cell may be in vitro. A cell may be in vivo. A cell may be ex vivo. A cell may be an isolated cell. A cell may be a cell inside of an organism. A cell may be an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell.

A cell may be from a specific organ or tissue. Non-limiting examples of organs and tissues from which a cell may be obtained or in which a cell may be located include: muscle, adipose, bone, adrenal gland, pituitary gland, thyroid gland, pancreas, testes, ovaries, uterus, heart, lung, aorta, smooth vasculature, endometrium, brain, neurons, spinal cord, kidney, liver, esophagus, stomach, intestine, colon, bladder, and spleen.

The tissue may be the subject's blood, bone marrow, or cord blood. The tissue may be heterologous donor blood, cord blood, or bone marrow. The tissue may be allogenic blood, cord blood, or bone marrow. In some embodiments, the cell is a stem cell. Non-limiting examples of stem cells are hematopoietic stem cells, muscle stem cells (also referred to as myoblasts or muscle progenitor cells), and pluripotent stem cells (including induced pluripotent stem cells). In some embodiments, the cell is cell derived or differentiated from a pluripotent stem cell. In some embodiments, the cell is a hepatocyte.

In some instances, the cell is an immune cell. Non-limiting examples of immune cells are lymphocytes (T cells, B cells, and NK cells), neutrophils, and monocytes/macrophages.

### Pharmaceutical Compositions and Modes of Administration

Disclosed herein, in some embodiments, are pharmaceutical compositions for modifying a target nucleic acid in a cell or a subject, comprising any one of the effector proteins, engineered effector proteins, fusion effector proteins, or guide nucleic acids as described herein and any combination thereof. Also disclosed herein, in some aspects, are pharmaceutical compositions comprising a nucleic acid encoding any one of the effector proteins, engineered effector proteins, fusion effector proteins, or guide nucleic acids as described herein and any combination thereof. In some embodiments, pharmaceutical compositions comprise a plurality of guide nucleic acids. Pharmaceutical compositions may be used to modify a target nucleic acid or the expression thereof in a cell in vitro, in vivo or ex vivo.

In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein. The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein.

In some embodiments, pharmaceutical compositions comprise a virus comprising a viral vector encoding a fusion effector protein, an effector protein, a fusion partner, a guide nucleic acid, or a combination thereof; and a pharmaceutically acceptable carrier or diluent. The virus may be a lentivirus. The virus may be an adenovirus. The virus may be a non-replicating virus. The virus may be an adeno-associated virus (AAV). The viral vector may be a retroviral vector. Retroviral vectors may include gamma-retroviral vectors such as vectors derived from the Moloney Murine Leukemia Virus (MoMLV, MMLV, MuLV, or MLV) or the Murine Stem cell Virus (MSCV) genome. Retroviral vectors may include lentiviral vectors such as those derived from the human immunodeficiency virus (HIV) genome. In some embodiments, the viral vector is a chimeric viral vector, comprising viral portions from two or more viruses. In some embodiments, the viral vector is a recombinant viral vector.

In some embodiments, the viral vector is an AAV. The AAV may be any AAV known in the art. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, and an AAV12 serotype. In some embodiments the AAV vector is a recombinant vector, a hybrid AAV vector, a chimeric AAV vector, a self-complementary AAV (scAAV) vector, a single-stranded AAV or any combination thereof. scAAV genomes are generally known in the art and contain both DNA strands which can anneal together to form double-stranded DNA.

In some embodiments, methods of producing delivery vectors herein comprise packaging a nucleic acid, or transgene, encoding an effector protein and a nucleic acid that, when transcribed, produces a guide nucleic acid, or a combination thereof, into an AAV vector. In some embodiments, methods of producing the delivery vector comprises, (a) contacting a cell with at least one nucleic acid, or transgene that, when transcribed, produces a guide nucleic acid; at least one nucleic acid that encodes: (i) a Replication (Rep) gene; and (ii) a Capsid (Cap) gene that encodes an AAV capsid protein; (b) expressing the AAV capsid protein in the cell; (c) assembling an AAV particle; and (d) packaging a Cas effector encoding nucleic acid into the AAV particle, thereby generating an AAV delivery vector. In some embodiments, promoters, stuffer sequences, and any combination thereof may be packaged in the AAV vector. In some embodiments, the AAV vector comprises a sequence encoding a guide nucleic acid. In some embodiments, the guide nucleic acid comprises a crRNA. In some embodiments, the guide nucleic acid is a crRNA. In some embodiments, the guide nucleic acid comprises a sgRNA. In some embodiments, the guide nucleic acid is a sgRNA. In some examples, the AAV vector can package 1, 2, 3, 4, or 5 nucleotide sequences encoding guide nucleic acids or copies thereof. In some examples, the AAV vector packages 1 or 2 nucleotide sequences encoding guide nucleic acids or copies thereof. In some embodiments, the AAV vector packages a nucleotide sequence encoding a first guide nucleic acid and a nucleotide sequence encoding a second guide nucleic acid, wherein the first guide nucleic acid and the second guide nucleic acid are the same. In some embodiments, the AAV vector packages a nucleotide sequence encoding a first guide nucleic acid and a nucleotide sequence encoding a second guide nucleic acid, wherein the first guide nucleic acid and the second guide nucleic acid are different. In some embodiments, the AAV vector comprises inverted terminal repeats, e.g., a 5′ inverted terminal repeat and a 3′ inverted terminal repeat. In some embodiments, the inverted terminal repeat comprises inverted terminal repeats from AAV. In some embodiments, the inverted terminal repeat comprises inverted terminal repeats of ssAAV vector or scAAV vector. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some embodiments, the AAV vector may be a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the delivery vector may be a eukaryotic vector, a prokaryotic vector (e.g., a bacterial vector) a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some embodiments, the genetic elements may be assembled by restriction digest of the desired genetic sequence from a donor plasmid or organism to produce ends of the DNA which may then be readily ligated to another genetic sequence.

In some embodiments, the vector is a non-viral vector, and a physical method or a chemical method is employed for delivery into the somatic cell. Exemplary physical methods include electroporation, gene gun, sonoporation, magnetofection, or hydrodynamic delivery. Exemplary chemical methods include delivery of the recombinant polynucleotide via liposomes such as, cationic lipids or neutral lipids; dendrimers; nanoparticles; or cell-penetrating peptides.

In some embodiments, a fusion effector protein as described herein is inserted into a vector. In some embodiments, the vector comprises a transgene which comprises a nucleotide sequence of one or more promoters, enhancers, ribosome binding sites, RNA splice sites, polyadenylation sites, a replication origin, and/or transcriptional terminator sequences.

In some embodiments, the AAV vector comprises a self-processing array system for guide nucleic acid. Such a self-processing array system refers to a system for multiplexing, stringing together multiple guide nucleic acids under the control of a single promoter. In general, plasmids and vectors described herein comprise at least one promoter. In some embodiments, the promoters are constitutive promoters. In other embodiments, the promoters are inducible promoters. In additional embodiments, the promoters are prokaryotic promoters (e.g., drive expression of a gene in a prokaryotic cell). In some embodiments, the promoters are eukaryotic promoters, (e.g., drive expression of a gene in a eukaryotic cell). Exemplary promoters include, but are not limited to, CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, 7SK, and HSV TK promoter. In some embodiments, the promoter is CMV. In some embodiments, the promoter is EF1a. In some embodiments, the promoter is U6. In some embodiments, the promote is H1. In some embodiments, the promoter is 7SK. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, the AAV vector comprises a promoter for expressing effector proteins. In some embodiments, the promoter for expressing effector protein is a site-specific promoter. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, the AAV vector comprises a stuffer sequence. A stuffer sequence can refer to a non-coding sequence of nucleotides that adjusts the length of the viral genome when inserted into a vector to increase packaging efficiency, increase overall viral titer during production, increase transfection efficacy, increase transfection efficiency, and/or decrease vector toxicity. In some embodiments, the stuffer sequence comprises 5′ untranslated region, 3′ untranslated region or combination thereof. In some embodiments, a stuffer sequence serves no other functional purpose than to increase the length of the viral genome. In some embodiments, a stuffer sequence may increase the length of the viral genome as well as have other functional elements

In some embodiments, the 3′-untranslated region comprises a nucleotide sequence of an intron. In some embodiments, the 3′-untranslated region comprises one or more sequence elements, such as an intron sequence or an enhancer sequence. In some embodiments, the 3′-untranslated region comprises an enhancer. In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-US' segment in LTR of HTLV-I (Mol. Cell. Biol., Vol. 8(1), p. 466-472, 1988); SV40 enhancer; the intron sequence between exons 2 and 3 of rabbit β-globin (Proc. Natl. Acad. Sci. USA., Vol. 78(3), p. 1527-31, 1981); and the genome region of human growth hormone (J Immunol., Vol. 155(3), p. 1286-95, 1995). In some embodiments, the enhancer is WPRE.

In some embodiments, the AAV vector comprises one or more polyadenylation (poly A) signal sequences. In some embodiments, the polyadenlyation signal sequence comprises hGH poly A signal sequence. In some embodiments, the polyadenlyation signal sequence comprises sv40 poly A signal sequence.

Pharmaceutical compositions described herein may comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO3. In some embodiments, the salt is Mg2+ SO42-.

Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives.

In some embodiments, pharmaceutical compositions are in the form of a solution (e.g., a liquid). The solution may be formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH of the solution is less than 7. In some embodiments, the pH is greater than 7.

In some embodiments, pharmaceutical compositions comprise an: effector protein, fusion effector protein, fusion partner, a guide nucleic acid, or a combination thereof; and a pharmaceutically acceptable carrier or diluent. In some embodiments, pharmaceutical compositions comprise one or more nucleic acids encoding an: effector protein, fusion effector protein, fusion partner, a guide nucleic acid, or a combination thereof; and a pharmaceutically acceptable carrier or diluent.

In some embodiments, guide nucleic acid can be a plurality of guide nucleic acids. In some embodiments, the effector protein comprises a sequence that is at least 65%, at least 70%, at least 75%, at least 80%, at least 85%, at least 90%, at least 95%, at least 97%, or at least 98%, at least 99%, or 100% identical to any one of the effector sequences of TABLE 1 and TABLE 2. In some embodiments, the effector protein comprises at least one amino acid alteration relative to the amino acid sequence of SEQ ID NO: 1. In some embodiments, the at least one amino acid alteration comprises an amino acid alteration set forth in TABLE 1.1. In some embodiments, the effector protein comprises at least one amino acid alteration relative to the amino acid sequence of SEQ ID NO: 2. In some embodiments, the at least one amino acid alteration comprises an amino acid alteration set forth in TABLE 1.2. In some embodiments, the guide nucleic acid comprises a nucleotide sequence of any one of the guide sequences of TABLE 1, TABLE 6, TABLE 8, or any combination thereof. In some embodiments, the PAM nucleic acid comprises a nucleotide sequence of any one of the PAM sequences of TABLE 1, TABLE 6, TABLE 8, or any combination thereof.

In some embodiments, the methods of this invention include using an inhibitor of DNA synthesis. In some embodiments the inhibitor is an inhibitor of replication fork regression. In some embodiments the inhibitor is an inhibitor of single strand break repair. In some embodiments the inhibitor is an inhibitor of double-strand break repair. In some embodiments the inhibitor is an inhibitor of non-homologous enjoining. In some embodiments the inhibitor is an inhibitor of RAD51. In some embodiments the inhibitor is AZD7648.

### Sequences and Tables

TABLE 1 provides exemplary effector protein amino acid sequences, exemplary guide protein binding sequences, and exemplary PAM sequences. With regards to the PAM sequences: N is any nucleotide. V is A. C. or G; Bis C. G. or T; and S is G or C.

TABLE 1
Exemplary Effector Protein Amino Acid Sequences, Exemplary Guide Protein
Binding Sequences, and Exemplary PAM Sequences

| | CasM.265466 | Casφ.12 |
|---|---|---|
| Exemplary | MSVLTRKVQLIPVGDKEERDRVYKYLRDGI | MIKPTVSQFLTPGFKLIRNHSRTAGLKLKNEGEEACKKFVRENEIPKD |
| Effector | EAQNRAMNLYMSGLYFAAINEASKEDRKE | ECPNFQGGPAIANIIAKSREFTEWEIYQSSLAIQEVIFTLPKDKLPEPIL |
| Protein | LNQLYSRIATSSKGSAYTTDIEFPTGLASTST | KEEWRAQWLSEHGLDTVPYKEAAGLNLIIKNAVNTYKGVQVKVDN |
| Amino | LSMAVRQDFTKSLKDGLMYGRVSLPTYRK | KNKNNLAKINRKNEIAKLNGEQEISFEEIKAFDDKGYLLQKPSPNKSI |
| Acid | DNPLFVDVRFVALRGTKQKYNGLYHEYKS | YCYQSVSPKPFITSKYHNVNLPEEYIGYYRKSNEPIVSPYQFDRLRIPI |
| Sequence | HTEFLDNLYSSDLKVYIKFANDITFQVIFGN | GEPGYVPKWQYTFLSKKENKRRKLSKRIKNVSPILGIICIKKDWCVFD |
| | PRKSSALRSEFQNIFEEYYKVCQSSIQFSGT | MRGLLRTNHWKKYHKPTDSINDLFDYFTGDPVIDTKANVVRFRYK |
| | KIILNMAMDIPDKEIELDEDVCVGVDLGIAI | MENGIVNYKPVREKKGKELLENICDQNGSCKLATVDVGQNNPVAIG |
| | PAVCALNKNRYSRVSIGSKEDFLRVRTKIR | LFELKKVNGELTKTLISRHPTPIDFCNKITAYRERYDKLESSIKLDAIK |
| | NQRKRLQTNLKSSNGGHGRKKKMKPMDR | QLTSEQKIEVDNYNNNFTPQNTKQIVCSKLNINPNDLPWDKMISGTH |
| | FRDYEANWVQNYNHYVSRQVVDFAVKNK | FISEKAQVSNKSEIYFTSTDKGKTKDVMKSDYKWFQDYKPKLSKEV |
| | AKYINLENLEGIRDDVKNEWLLSNWSYYQ | RDALSDIEWRLRRESLEFNKLSKSREQDARQLANWISSMCDVIGIEN |
| | LQQYITYKAKTYGIEVRKINPYHTSQRCSC | LVKKNNFFGGSGKREPGWDNFYKPKKENRWWINAIHKALTELSQN |
| | CGYEDAGNRPKKEKGQAYFKCLKCGEEM | KGKRVILLPAMRTSITCPKCKYCDSKNRNGEKFNCLKCGIELNADID |
| | NADFNAARNIAMSTEFQSGKKTKKQKKEQ | VATENLATVAITAQSMPKPTCERSGDAKKPVRARKAKAPEFHDKLA |
| | HENK (SEQ ID NO: 1) | PSYTVVLREAV (SEQ ID NO: 2) |
| Exemplary | ACAGCUUAUUUGGAAGCUGAAAUGUGA | AUUGCUCCUUACGAGGAGAC (SEQ ID NO: 6) |
| Protein | GGUUUAUAACACUCACAAGAAUCCUGA | |
| Binding | AAAAGGAUGCCAAAC (SEQ ID NO: 4); | |
| Sequences | ACAGCUUAUUUGGAAGCUGAAAUGUGA | |
| | GGUUUAUAACACUCACAAGAAUCCUGA | |
| | AAAGGAUGCCAAAC (SEQ ID NO: 283) | |
| Exemplary | NNTN; TNTR; TNTG: NNTR | NTYN; NTTN; TTTS |
| PAMs | | |

TABLE 1.1 provides exemplary amino acid alterations relative to SEQ ID NO: 1 useful in compositions, systems, and methods described herein.

TABLE 1.1
Exemplary Amino Acid Alterations Relative to SEQ ID NO: 1

| Effects | Amino Acid Alterations |
|---|---|
| | At least one substitution (i.e., with R, K or H) selected from K58, I80, T84, K105, N193, |
| | C202, S209, G210, A218, D220, E225, C246, N286, M295, M298, A306, Y315, and Q360 |
| Enhanced nuclease | I80R, T84R, K105R, C202R, G210R, A218R, D220R, E225R, C246R, Q360R, I80K, T84K, |
| activity relative to the | G210K, N193K, C202K, A218K, D220K, E225K, C246K, N286K, A306K, Q360K, I80H, |
| wild-type effector | T84H, K105H, G210H, C202H, A218H, D220H, E225H, C246H, Q360H, K58W, S209F, |
| protein | M295W, M298L, Y315M |
| | Double mutations: D220R/A306K, D220R/K250N |
| dCas | D237A, D418A, D418N, E335A, E335Q |

* Reduced or abolished nuclease activity relative to the wild-type effector protein

TABLE 1.2 provides exemplary amino acid alterations relative to SEQ ID NO: 2 useful in compositions, systems, and methods described herein.

TABLE 1.2
Exemplary Amino Acid Alterations Relative to SEQ ID NO: 2

| Effects | Amino Acid Alterations |
|---|---|
| | At least one substitution (i.e., with R, K or H) selected from I2, T5, K15, R18, |
| | H20, S21, L26, N30, E33, E34, A35, K37, K38, R41, N43, Q54, Q79R, K92E, K99R, |
| | S108, E109, H110, G111, D113, T114, P116, K118, E119, A121, N132, K135, Q138, |
| | V139, N148, L149, E157, E164, E166, E170, Y180, L182, Q183, K184, S186, K189, |
| | S196, S198, K200, I203, S205, K206, Y207, H208, N209, Y220, S223, E258, K281, |
| | K348, N355, S362, N406, K435, I471, I489, Y490, F491, D495, K496, K498, K500, |
| | D501, V502, K504, S505, D506, V521, N568, S579, Q612, S638, F701, and P707 |
| Enhanced | T5R, L26R, L26K, A121Q, N148R, V139R, S198R, H208R, S223P, E258K, N355R, I471T, |
| nuclease | S579R, F701R, P707R, D703G, K189P, S638K, Q54R, Q79R, Y220S, N406K, E119S, |
| activity | K92E, K435Q, N568D, and V521T |
| relative to | Double mutations: L26K/A121Q, L26X/A121Q, K99R/L149R, K99R/N148R, L149R/H208R, |
| the wild- | S362R/L26X L26X/N148R, L26X/H208R, N30R/N148R, L26X/K99R, L26X/P707R, |
| type effector | L26X/L149R, L26X/N30R, L26X/N355R, L26X/K281R, L26X/S108R, L26X/K348R, T5R/V139R, |
| protein | I2R/V139R, K99R/S186R, L26X/A673G, L26X/Q674R, S579R/L26K, F701R/E258K, |
| | T5R/L26K, L26X/K435Q, L26X/G685R, L26X/Q674K, L26X/P699R, L26X/T70E, L26X/Q232R, |
| | L26X/T252R, L26X/P679R, L26X/E83K, L26X/E73P, L26X/K248E, L26X, T5R/S223P, |
| | S579R/S223P, L26X/S223P, T5R/A121Q, L26X/A696R, S198R/I471T, L26X/N153R, |
| | L26X/E682R, L26X/D703R, Q612R/L26K, L26X/I471T, K348R/L26K, S579R/1471T, |
| | L26X/V228R, T5R/S638K, S579R/K189P, S579R/E258K, L26X/K260R, L26X/S638K, |
| | S579R/Y220S, T5R/I471T, L26X/F233R, L26X/V521T, F701R/A121Q, L26X/G361R, |
| | S198R/E258K, L26X/S472R, T5R/Y220S, L26X/A150K, L26X/S684R, L26X/E157R, |
| | L26X/K248R, F701R/L26K, S198R/N406K, S198R/Y220S, S198R/S638K, S198R/V521T, |
| | S579R/A121Q, K348R/Y220S, S198R/K189P, L26X/E242R, L26X/K678R, T5R/N406K, |
| | L26X/I158K, T5R/V521T, L26X/N259R, L26X/K257R, L26X/K256R, T5R/K189P, |
| | L26X/C405R, S579R/V521T, S579R/N406K, T5R/K92E, T5R/E258K, L26X/I97R, |
| | S579R/S638K, T5R/K435Q, F701R/S638K, L26X/L236R, F701R/I471T, Q612R/S223P, |
| | F701R/S223P, S198R/E119S, S579R/K92E, L26X/E715R, Q612R/I471T, F701R/Y220S, |
| | S198R/S223P, and L26X/K266R, wherein X is selected from R and K. |
| | Quadruple mutations: L26R/1471T/S223P/D703G |
| Nickase | E157A, E164A, E164L, E166A, E166I, E170A, I489A, I489S, Y490S, Y490A, F491A, |
| activity | F491S, F491G, D495G, D495R, D495K, K496A, K496S, K498A, K498S, K500A, K500S, |
| | D501R, D501G, D501K, V502A, V502S, K504A, K504S, S505R, D506A; deletion of |
| | S478-S505 of SEQ ID NO: 2; deletion of S478-S505 of SEQ ID NO: 2 and insertion |
| | of the sequence of SDLYIERGGDPRDVHQQVETKPKGKRKSEIRILKIR (SEQ ID NO: 7); deletion |
| | of S478-S505 of SEQ ID NO: 2 and insertion of the sequence of SDYIVDHGGDPEKVFF |
| | ETKSKKDKTKRYKRR (SEQ ID NO: 8); an amino acid sequence that is at least 90%, |
| | at least 95%, at least 97%, at least 98%, at least 99% identical, or is 100% |
| | identical to SEQ ID NO: 9; an amino acid sequence that is at least 90%, at |
| | least 95%, at least 97%, at least 98%, at least 99% identical, or is 100% |
| | identical to SEQ ID NO: 10 |
| dCas* | D369A, D369N, D658A, D658N, E567A, E567Q |

*Reduced or abolished nuclease activity relative to WT effector protein

TABLE 2 provides exemplary effector protein CasPhi. 12 engineered variant sequences.

TABLE 2
Exemplary Effector Protein CasPhi.12 Engineered Variant Amino Acid Sequences

| Effector | | SEQ |
|---|---|---|
| Protein | | ID |
| Variant | Amino Acid Sequence | NO: |
| j12_L17_ | MIKPTVSQFLTPGFKLIRNHSRTAGLKLKNEGEEACKKFVRENEIPKDECPNFQGGPAIANIIAKSREFTEWEIYQSSL | 9 |
| 18_del | AIQEVIFTLPKDKLPEPILKEEWRAQWLSEHGLDTVPYKEAAGLNLIIKNAVNTYKGVQVKVDNKNKNNLAKINRK | |
| 1 | NEIAKLNGEQEISFEEIKAFDDKGYLLQKPSPNKSIYCYQSVSPKPFITSKYHNVNLPEEYIGYYRKSNEPIVSPYQFD | |
| | RLRIPIGEPGYVPKWQYTFLSKKENKRRKLSKRIKNVSPILGIICIKKDWCVFDMRGLLRTNHWKKYHKPTDSINDL | |
| | FDYFTGDPVIDTKANVVRFRYKMENGIVNYKPVREKKGKELLENICDQNGSCKLATVDVGQNNPVAIGLFELKKV | |
| | NGELTKTLISRHPTPIDFCNKITAYRERYDKLESSIKLDAIKQLTSEQKIEVDNYNNNFTPQNTKQIVCSKLNINPNDL | |
| | PWDKMISGTHFISEKAQGSSGDYKWFQDYKPKLSKEVRDALSDIEWRLRRESLEFNKLSKSREQDARQLANWISSM | |
| | CDVIGIENLVKKNNFFGGSGKREPGWDNFYKPKKENRWWINAIHKALTELSQNKGKRVILLPAMRTSITCPKCKYC | |
| | DSKNRNGEKFNCLKCGIELNADIDVATENLATVAITAQSMPKPTCERSGDAKKPVRARKAKAPEFHDKLAPSYTVV | |
| | LREAV | |
| j12_L17_ | MIKPTVSQFLTPGFKLIRNHSRTAGLKLKNEGEEACKKFVRENEIPKDECPNFQGGPAIANIIAKSREFTEWEIYQSSL | 10 |
| 18_del | AIQEVIFTLPKDKLPEPILKEEWRAQWLSEHGLDTVPYKEAAGLNLIIKNAVNTYKGVQVKVDNKNKNNLAKINRK | |
| 2 | NEIAKLNGEQEISFEEIKAFDDKGYLLQKPSPNKSIYCYQSVSPKPFITSKYHNVNLPEEYIGYYRKSNEPIVSPYQFD | |
| | RLRIPIGEPGYVPKWQYTFLSKKENKRRKLSKRIKNVSPILGIICIKKDWCVFDMRGLLRTNHWKKYHKPTDSINDL | |
| | FDYFTGDPVIDTKANVVRFRYKMENGIVNYKPVREKKGKELLENICDQNGSCKLATVDVGQNNPVAIGLFELKKV | |
| | NGELTKTLISRHPTPIDFCNKITAYRERYDKLESSIKLDAIKQLTSEQKIEVDNYNNNFTPQNTKQIVCSKLNINPNDL | |
| | PWDKMISGTHFISEKAQVSNKSEGSSGDYKWFQDYKPKLSKEVRDALSDIEWRLRRESLEFNKLSKSREQDARQLA | |
| | NWISSMCDVIGIENLVKKNNFFGGSGKREPGWDNFYKPKKENRWWINAIHKALTELSQNKGKRVILLPAMRTSITC | |
| | PKCKYCDSKNRNGEKFNCLKCGIELNADIDVATENLATVAITAQSMPKPTCERSGDAKKPVRARKAKAPEFHDKL | |
| | APSYTVVLREAV | |
| L26R, | MIKPTVSQFLTPGFKLIRNHSRTAGRKLKNEGEEACKKFVRENEIPKDECPNFQGGPAIANIIAKSREFTEWEIYQSSL | 1607 |
| I471T, | AIQEVIFTLPKDKLPEPILKEEWRAQWLSEHGLDTVPYKEAAGLNLIIKNAVNTYKGVQVKVDNKNKNNLAKINRK | |
| S223P, | NEIAKLNGEQEISFEEIKAFDDKGYLLQKPSPNKSIYCYQSVSPKPFITSKYHNVNLPEEYIGYYRKPNEPIVSPYQFD | |
| D703G | RLRIPIGEPGYVPKWQYTFLSKKENKRRKLSKRIKNVSPILGIICIKKDWCVFDMRGLLRTNHWKKYHKPTDSINDL | |
| | FDYFTGDPVIDTKANVVRFRYKMENGIVNYKPVREKKGKELLENICDQNGSCKLATVDVGQNNPVAIGLFELKKV | |
| | NGELTKTLISRHPTPIDFCNKITAYRERYDKLESSIKLDAIKQLTSEQKIEVDNYNNNFTPQNTKQIVCSKLNINPNDL | |
| | PWDKMTSGTHFISEKAQVSNKSEIYFTSTDKGKTKDVMKSDYKWFQDYKPKLSKEVRDALSDIEWRLRRESLEFN | |
| | KLSKSREQDARQLANWISSMCDVIGIENLVKKNNFFGGSGKREPGWDNFYKPKKENRWWINAIHKALTELSQNKG | |
| | KRVILLPAMRTSITCPKCKYCDSKNRNGEKFNCLKCGIELNADIDVATENLATVAITAQSMPKPTCERSGDAKKPVR | |
| | ARKAKAPEFHGKLAPSYTVVLREAV | |

TABLE 3 provides exemplary RDDP sequences.

TABLE 3
Exemplary RDDP Sequences

| RDDP | | SEQ |
|---|---|---|
| Ref. | | ID |
| No. | Amino Acid Sequence | NO: |
| 2691297 | VNHYLRLTSLPALYAGWERVAANDGGPGVDRVSINKFDVALDRDLERLHRELVSFTYRCMPLRRYEIPKLNGKIR | 11 |
| | TLAVPTVRDRVVQSSALIVLQPVIERELERCSFAYRPGLSRLTAIEQIRQLREQGYRWVVDADIESFFDNVEFGLLL | |
| | RRFRELVPENHTVALIEQWLKAPVLFHGRLIKRTKGLPQGSPISPVLANLFLDKFDEALLAHNHKLIRYADDFIILCK | |
| | TKPRAEEALRLTEELLAGLHLRLNAEKTAVTSFDQGFKYLGAIFVKSIIIPQSGTKPQRRKKQMNAQSPDPPLRQNE | |
| | KRRKSAAAKKKEEITVLAQALLEALNAKQMTTADLVRTPPTRPPVPNGAPEPPSAPPNVSPFMRTLYIQEQGCWL | |
| | RLDGERFVVATGGEEGVALYEIPTVKVEQIMIFGTCLITPAAMRYALLHTIPITLLSSRGQYFGRIESTYGADITLERT | |
| | QFLHSVDTAFVLQTARTIVQAKLRNTKALLQRHQTKHEMISAAIVQLTRLEDQVANATSVDQVRGYEGSGSAVFF | |
| | DVFDELMPGKGFTFEKRTRRPPTDPVNAMLSFGYTLLFNNIYSMARLHRLNPYVGSLHAALSEHKQALTCSY | |
| 2691299 | MYRCVKLPVAENMPNTDSQLNTVGWDEIDWRKVERYVYKLHKRIYAASRRGDVKQVRKLQKTLMSSWSNQVL | 12 |
| | SVRKVTQDNRGKRTAGVDGIHILSSEARINLAGSLKITGKSHPTRRVWIPKPGTEEKRPLGIPTIYDRVVQNLVKTA | |
| | LEPEWEAIFEPNSFGFRPGRSCHDAIWQIKNSIQNRPKFILDADIAKCFDRINHLALLQKLGYTGKIRQQIKAWLKSG | |
| | VIDRGAFTATSEGTMQGGVISPLLANIALHGMEKMLMEFAKTLDMRRNDKPNCQISWQQKVKSLTFVRYADDFV | |
| | LIHKDLNVVRRCRDLISEWLKDIGLELKPSKTRIAHTKKIRERGSPEAKRYLYRGGNATRA | |
| 2691301 | MMAHRKIKANHGSKTKGMDKQNVIDFKNMVTEEYLSEIKLELLDYKSGLIRRKEIPKPNGGVRPLGISCYKDKIIE | 13 |
| | EAIKQVIEPIVEAKFHPDSYGFRPGRSAQEAIQMSQTLVRAGYTYACEFDIKKCFDNIKHRLIRKKLWALGIRDTRL | |
| | LTIISKRLRANIYDPKTKTVSQSTLGTVQGGVLSPLLANVVLNSLDWWIASQWERFDFGIEENDYKNYASFHAAM | |
| | NYRLNKTRLKSGRIVRYADDFVIFCKTEMEAKAWYFAVKDWMKCNLKLEISEEKSRIVDLTKDSIDFLGFELKAN | |
| | KEGKKCVKSVDRSNRGSYKYTKTSVTTKNLNKIIQTLKTKIKIILKTSDKKAKAVLITLLNITILGYHNYWRIASESS | |
| | INFYNIYQRTKLSWWKLRRQADSKTHISPAFRKLYKEYVDSAFSIDGNTIYPIWGCRHTTLNPKKRDYNWYEDSPS | |
| | IPENISSQMKIMCCNPIIHRSVEYNDNRLSKYSMQLGKDYITGKFVMAQDVHCHHKTMVSKGGKDNFDNLVILSK | |
| | VTHGWVHQVNPEIPYMTKVQMDRLNDLREKCGREPLKNKKKSLKSEN | |
| 2691303 | MRKWYSLIDKVYARANLLEAFDKVRRNKGGKTSGIDGISVQAFKEHLTANLCQLHEELRSGTYRPQAVKRVYID | 14 |
| | KEDGGKRPLGIPTVKDRVVQQALLNVLQPIFEPEFHPSSYGYRPGRSAHHAIAKAERFTRYYGLDHVVVMDLSKC | |
| | FDTLDHELILSSVNEKVSDGKVLRLIDSFLKSGIIDKGKFEPTEKGSPQGGIISPLLTNIYLNKFDQYMKTLGIRIVRY | |
| | ADDILVFASTKSEAGKYRAIATTYLEGELMLTVNREKTHLTTPYKGIPYLGFEIHNYGVSASEKSIRKFKEKVRKLT | |
| | PRNQGYKLDYYLTELNFLLKGYSMYFRVARSKSLFRNLMSWVRRRLRMMIMKSWKSWKGLHKQLRRMGYKGS | |
| | LEKISVTRWRNSSCQLLHMALPNTWFDEQHLFNMEKVTTGTLHQYYNFVLNKV | |
| 2691305 | VNRAVLTLADLARPDALFDAWQQAERRKAGPGTDGQTVQVFGQQVEEHLEGLSKEIREGGYQPRPAQRIWMVK | 15 |
| | DSGGERGITIFTVRDRVALGAMRRTLGRLIGPTLSTASFAYREGLGALRAAHRLIEHRRSGRGWVVRSDIKEFFDTI | |
| | SHEILLEQLRVFLDARALELFGTILRTPIRDGYGISVPERGVAQGSPISPMLANLYLSGFDAALNCDGRGFVRFADDF | |
| | VIAALSVKGAAEGLEIAQAQLEGLELRLNDDKTRIVSFEQGFDFLGFHFDADTVRVSETSLAEFKAHLEALLVSRFE | |
| | QFSPGAVKKANDLIRGWRNYFQLGDVRKDYAALEEWIEARFGHKARQLERLLPDARGQRVPQLGGYGRVSRER | |
| | QPRRSGQANTASARPQKRQRPYPNVKLDANDTPLTVRRNKAVLNVATLPALPETRVSRAILDELEQASLFHRANT | |
| | ACRWSTPDAADAVSNLAAAMSGRIPIRQAIEAYDKALFAQLKPGADPRGARGTLRANLRRIAWAALLEAGIDLGP | |
| | EQPRKTPTLGNALADAYECLTADLALLAASRNDQLHAPQGAFERSLAFKPAYQRPGVQGQAHATAWMMILRLE | |
| | AEAMRRMLETNGQTVYRVWRFGLP | |
| 2691307 | MEAVVERENMIAAYKRVVANRGAAGVDGMTVDALKAYLQPHWPRIRKELLEGRYRPQPVLEVEIPKPAGGMRK | 16 |
| | LGIPTVVDRLIQQAIHQVLEPIFDKDFSESSYGFRRGKSAHQAVKQAREYVKEGRRWVVDIDLEKFFDRVNHDILM | |
| | ARVARKVRDKRILRTIRQYLQAGIMTGGLVTARTEGTPQGSPLSPLLSNIMLDDWDKELERRGHRFCRYADDCNV | |
| | YVHSKRAGERVMESLRQFLEKRLRLRVNTEKSRVDHPWQLKFLGYSMTWEKNTRLKVASQSVERFKSKLRLIFR | |
| | QGRGRNVQIVINELNLVLRGWVQYFRLAEVKNIFEEMDGWIRRKLRCTLWKQWKRTYTRAKNLMKRGLEEVRA | |
| | WQSATNGRGAWWNSAASHMNEAYPKSYFDKAGLVSLLQLLWQIQLT | |
| 2691309 | MSEPMEDKPEAVSERIKRAGELLRHWMWTEPTVWTERMLTALAQGVKGGKWFSLIDKIHPERTLRAAYGHVAA | 17 |
| | NKGSAGVDHVTTTMFGDHLDEEVSKLSEQLRNGSYRPQAIRRHYIPKPGSQEKRPLGIPTVRDRVVQTALRMVIEP | |
| | IFEREFAEDSYGFRPGRGCKDALRRVDELLKDGYIYIIDADLKSYFDTIPHDRLMDLVRQKISDGRVLNLIEAFLKQ | |
| | GILENLQEWIPESGSPQGAVVSPLLSNIYLDPLDHKMAQEGYQMVRYADDFVILCRTPEKAERALAAVQEWTAAA | |
| | GLTLHPTKTRIVNATIDSFEFLGYRFSKGKRFPRTKSMQKLKDTIRAKTKRTNGQCLPAIIGSLNPTLRGWFEYFKH | |
| | SYKTTFPELDGWIRGRLRSILRKRQKKRGRGRGSDHQRWPNAYFADLGLYSLKTAHKLACQSSSR | |
| 2691311 | MKGRMQKKSIDTCQQKNRTESDGYVEGQTFIWITENNLTDKYQLGNGLLEFILSPNNLNQAYHQVKRNKGAGGI | 18 |
| | DKMEVESLKDYLVRHKDELIKSILEGKYRPSPVRRVEIPKETGKTRQLGIPTVVDRVIQQSISQVLSQIYEKQFSDTS | |
| | YGFRPRRGAHQALRTCQKNITEGYVWSVDMDLEKFFDTVNHSKLIEVLSRTIKDGRVISLIHKYLNAGVIVRNNFE | |
| | ETEVGVPQGGPLSPLLSNIMLNELDREIENRGHRFVRYADDMVILCKSKRSAVRTLENLLPFIEGKLFLKVNKEKTS | |
| | VAQISKIKFLGYSFYRLNGECQLRVHPKSVLKMREKMKSLTSRSNGWGNERRKESLRQYIVGWVNYFKLANMKK | |
| | LLLRTDEWYRRRLRMVIWKQWKRISTKLINLIKLGINKYKAWELVNTRKGY | |
| 2691313 | MSLATPERIRSLQRKLYCKAKAEPAFRFYTLYDKICRADILAHAYEVARSNAGAPGVDGRTFEHIEALGLQEWLA | 19 |
| | GLREDLVARTYRPMPVRRVMIPKPGGGERPLGIPTIRDRVIQTAAKLVLEPIFEADFEDSAYGYRPRRGASDAIKEV | |
| | HRLLCRGHTDVADADLSKYFDTIPHAQLLRCVARRIVDGEVLRLIKLWLKTPIEGRDADGKRRLTGGKRSRCGTP | |
| | QGGVVSPLLANLYMNRFLKHWRFTGCGEVFRAQIIAYADDFVILSCGHAAEAMMWTKAVMTKLGLNLNEAKTSI | |
| | RDARKERFEFLGYSFGPHYWWKNGQRYLGASPSKKSVQRIKAKISELLVPGNKGSWPDVRDQLNRLLSGWSAYF | |
| | NHGTRVPAYRAVDAHVYDRVQNFLCRRHNVRRRGTSRFSFGDVFGELAVLHLM | |
| 2691315 | MDEDFLTEAFYQLRKDAAAGIDEMTATEYEVNLGGRIADLHRRLVAQEYRAQPARRVWIPKGDGGQRPLAIL VL | 20 |
| | EDKIVQRAVAMLLEAIYEPHFCGFSYGFRREHNAHQALTYLRQQCLELGINWIIDADIQKFFDTISWEQLRAILQRR | |
| | MNDGAVLRLIGMWLHVGVLEAGQVINQELGTPQGAPISPILANIFLHIVLDEWFQSQVRPRMRGNCFLVRYADDF | |
| | VMGYSLKGDAERVYEVLPKRFERYGLRIHPEKSRMVQFSRPYWQRGKGPGSFAFLGFTHYWAKTRSGSWTIKRK | |
| | TQGKRLSRFLSGIADWCKENLHEAIGEQHRILSAKLRGHYQYYGVRGNFKLLEVAYEHTRGMWKKWLGRRNSM | |
| | NRMSWEKFVEQVESVFTLPLPRIVHAF | |
| 2691317 | MPKTFYSLYDRLLHRRALARAFAKVRRAKGAKTPGQDGQTAADFAADEEAELDRLVHELRTKTYRPQPVRRVTI | 21 |
| | PKPGGGERNLGIPAVRDRVVQQSLLDILQPIFDPDFHPSSYGYRPGRSSQQAVAKATRFVRQYGLAHAVDMDLSK | |
| | CFDRLDHDLIVASFRRRVRDGSILALMRMFLESGVMIGDDWEATEVGSPQGGVISPLISNVYLDAFDQEMKRRGY | |
| | RIVRYADDILILCRSKSAALHAREVATAILEGPLRLTVNKEKTHLVHASQGVKFLGVEIGLTWTRIQDKKVAAFKA | |
| | QVKRLTRRNSPVNLGQVIADLNPVLRGFANYFRMANCKGLLSELMSWIRRRLRAKQLALWKKPGRLHRRLRQLG | |
| | YQGDFPKIRMTRWRNSRSLQASWSLPNGWFRELGLYELTAVETGVLPQAT | |
| 2691319 | MSELLEKILSKDNMNTAYKRVCANKGAGGVDDVTVEELGAYVKENWESIREQIRRREYTPQPVRRVEIPKPDGGE | 22 |
| | RKLGIPTVMDRVIQQGIVQIISPMCEPLFSKWSYGFRPNRSCEMAIRQLLEYLNEGYEWIVDIDLEKFFDNVPQDRL | |
| | MSLVHDIIKDGDTESLIRKYLKAGAMTPQGYKETKLGTPQGGNLSPLLSNIMLNELDKELEARGLRFTRYADDCVI | |
| | AVKSESSAKRVMRTVTDWIQRKLGLKVNMTKSQITRPQKLKYLGFGFHKDSKAKEWKCRPHQESVKKFKVRLK | |
| | ELTCRKKPGTVTSKIAKINQVTRGWINYYALGSMKTAMTEIDAHLRTRLRVIIWKQWKVPKKRQWGLQKLGVSK | |
| | DLARLTAYCGDSYYWVATKTCVVRAISKEVFAKSGLVSCLDYYNERHALKLC | |
| 2691321 | MEQVVERSNMLAALKRVERNGGAPGIDGVPTERLRDQIRAEWRRIREELLAGTYRPEPVRRVEIPKPGGGERLLGI | 23 |
| | PTVMDRLIQQALLQVLTPIFDPQFSDSSYGFRPGRRAHDAVKRARQYVEEGYEWAVDLDIEKFFDRVNHDILMAR | |
| | VARRVTDKRVLTLIRRYLQAGVMVNGVVMETAEGTPQGGPLSPLLANILLDDLDKELEKRGHKFVRYADDCNIY | |
| | VKSKRAGERVMASVRNFLQERLKLKINEQKSAVDRPWKLKFLGFSMYKPKGGVILIRLASQTIDRVKAKIRGITAR | |
| | NKPISMDERIERLNTYLGGWIGYFALADTPSVFKDIEGWMRRRLRMCLWKQWKRVRTRYRELRALGLPEWVVH | |
| | HFANARKGPWRMAHGPMNRALGNAYWQSHGLMSLTERYHRLRQAW | |
| 2691323 | MSELLERILSNDNMNAAYKRVCANKGAGGVDGVTVEELGDYIKENWSSIREQIRRRQYKPQPVRRVEIPKPNGGV | 24 |
| | RNLGIPTVMDRVIQQGIVQVLSPMCEPLFSERSYGFRPNRSCEMAIRQLLVYLNEGCEWIVDIDLEKFFDNVPQDRL | |
| | MSLVHDIINDGDTESLIRKYLKAGVMTPQGYEETKLGTPQGGNLSPLLSNIMLNELDKELEKRGLRFTRYADDCVI | |
| | AVKSESSAKRVMRTVADWIQRKLGLKVNMTKTHITRPTKLKYLGFGFYRDNKAKEWKCRPHQDSVAKFKRKLK | |
| | ELTCRKTPGTVTGRIAKINQVTRGWINYYAIGSMKTTMIEIDAHLRTRLR VIIWKQWKVPKKRQWGLQKLGIGKD | |
| | LARLTSYYGDRYQWIVTKTCVARAISKEVLAKAGLVSCLDYYNERHALKLC | |
| 2691325 | VKPREAGMGGPSSSLAQVQTPSCEEGDSLLEKMLERENLLLALRRVEANKGAPGVDGVTVAQLRSYIQTHWADI | 25 |
| | RQQLLAGTYKPQPVRRVEIPKPGGGVRGLGIPTVIDRFIQQALLQVLNPIFDPEFSDNSFGFRPGRSAHDAVRRAQQ | |
| | YIQQGYRWAVDLDLAKFFDRVNHDKLMARVARKVKDKRVLKLIRAYLNAGIMADGVVVRNEEGTPQGGPLSPL | |
| | LANIMLDDFDKELKKRGHRFVRYADDCNVYVKSRRAGERVMASLTRYLEGTLKLQVNQEKSAVDRPWKLKFLG | |
| | FSFLPDKLATIRLAPKTLERFKERVRQITSRSRSMPIAERIRQLNAYIMGWVAYYRLAEMKRHCERFDEWIRRRLR | |
| | MCIWKQWKRVRTRYRELRALGQPEWVVHMTANSRRGPWFMARMLNQAMDKTYFESTGTAQSSGEIPYTSWCF | |
| | MNRRMRTRTYGGVRGRGLGAPSYSIGGTSTRCIVAHRIVVTFGQCAPSGFTEVSGGR | |
| 2691327 | MEQLGGDPRYCLMEQVVERANMTKALRRVERNGGAAGVDGMEPEALRSYLKEHWPRIKEELLAGTYRPMPVR | 26 |
| | RVEIPKPGGGVRLLGIPTVLDRLIQQALLQVLTPIFDPGFSPHSYDFRPGRSAHQAVEQARRYVAQGYRHVVDLDL | |
| | AQFFDRVNHDLLMARVARKVKDKRVLKLIRAYLQAGVMINGCCVRTEEGTPQGGPLSPLLANIILDDLDKELERR | |
| | GHKFVRYADDSNVYVRSCRAGQRVFESLKRFLEQRLKLRINEEKSAVDYAWRRGILGFSFTWEKEPRIRLAPQTV | |
| | KRFKQRIRWLTKRSRSVNMAERLARLNEYLKGWMGYFRLIETPSTLQALDEWIRRRLRMCLLKQWKRPRTRRRN | |
| | LVALGIPEDWARHISGSRKGCWRLANTPQVNKALGLAYWRSQGLVSLVDRYRELRSAS | |
| 2691329 | VRSREGRRQPKTLNRNCPREEVVKPRGIVGEPSPSPAQSGTSPRGDQGDSLMEKVVARDNMLAALKRVEQNKGA | 27 |
| | PGVDGIPTENLREQIRAEWPRIREELLAGTYRPQPVRRVEIPKPGGGKRMLGIPTVMDRLIQQALLQVLTPIFDPEFS | |
| | EASYGFRPGRRAHDAVKKARQYVEEGYEWGVDMDLEKFFDRVNHDVLMARVARKVTDKRVLKVIRRYLQAGI | |
| | VVNGVVMEREEGTPQGGPLSPLLANILLDDLDKELEKRGHKFVRYADDANIYVRSRRAGERVLASVRKFLQERLK | |
| | LKLNEQKSTVDRPWKLKFLGFSMYKPKAGEIRIRLARQSIDRVKTKIRALTARNTPIAMEERIRRLNTYLGGWIGYF | |
| | ALAETPSIFEEIDGWIRRRLRMCLWKQWKRVRTRYRELRALGLPEWVVHPFANARKGPWRMAHGPMNRALGNA | |
| | YWQAQGLMSLTERYRRLRQAW | |
| 2691331 | MEKQSMIINNILSNDNLNHAYKQVVKNKGAAGIDGMECEALFSHLRTNGAQLKESIRNQAYNPLPVKRVEISKED | 28 |
| | GSKRNLGIPTVTDRLIQQAVAQVLTPIYEKVFHRNSYGFRPGKSAQQAVLKAVEYMNGGYNWVVDIDLEKFFDT | |
| | VNHNKLISILNKEIKDGKVLSLIRKFLVSGVMVGEHVEETEIGTPQGGNLSPLLANIILNELDWELEKRKLKFVRYA | |
| | DDCIILVRSQKAAQRVMDSISKYIESKLLLKVNRKKSKIGRPIEIKYLGFTFYNQFKAKKYKAKAHEKSVQKVVRK | |
| | LKQLTSRRWGVSNSVKAQKIAEVLRGWINYFKIGSILTITRKLDTMLRYRFRMCIWKHWKTPQNKYKNLVKLGV | |
| | EPRNARRAAWSHGYARICRTEAVCYAMSNARLVKFGLLSAEAYFVKVSS | |
| 2691333 | MHEPTLLSQIIHPANLNQAYKQVMKNKGAPGIDDMPITALKAHLALHKETLVHQLQRREYKPQPVKRVEIPKASG | 29 |
| | GVRLLGIPTVTDRFIQQAIAQVLTPIFDKQFHDNSYGFRPKRYAEMAILQALENMNEGYEWLVDIDLERFFDTVHH | |
| | DRLMNIVARTVTDGDVISLVRKFLVSGVMVQDEYQETIIGTPQGGNLSPLLSNIMLHELDKELANRDLRFVRYAD | |
| | DCLIFVKSDMAARRVMRSVSKFIEEKLGLIVNVTKSKVRRPEDEETKFLGFGFYYDWNDRLYKARPHKTSLQAIKF | |
| | KLKCLTRRNWGVPTKSRVERINSLLRGWVNYFKIGKMKKHLIQLDANIRVRLRMCIWKQWKTPKNRRKNLIKLG | |
| | MNRYEAYKYSYTSKSYARVAYSWILTTTITNQRLTKFGLISATEHYSNIHV | |
| 2691335 | MLDRDNLRLAYKRVVRNGGAPGVDGVTVAALQSYLNTHWDRVKNELLAGTYRPAPVRRVEIPKPGGGVRLLGI | 30 |
| | PTVMDRFLQQALLQVMNPIFDAHFSWYSYGFRPGKRAHDAVMQAQRYIRDGYRWVVDLDLEKFFDRVNHDML | |
| | MARVARKVKDKRVLKLIRAYLNAGVMINGVCQRTEEGTPQGGPLSPLLANILLDDLDKELTRRGLRFVRYADDC | |
| | NIFVASKRAGERVMESAIRFLEGKLKLKVNRDKSAVDRPWKRKFLGFSFLSDKEATIRLAPKTISRFKERVREITSR | |
| | SRPIPMEERIRRLNQYTMGWVSYFRLASMKNHCERFDQWIRRRLRMCLWKQWKRVRTRIRELRALGVPDWACF | |
| | AMANSRRGPWEMSRNINNALPTSYWEAKGLKSMLTRYMVLRQPFGTAWCGPACQVV | |
| 2691337 | VHTHEDRIALSSPPNQADLQIHTGTLSPYTSHWGYSFLDPSIQEAPLLGQSEGQFLPPNLFSYPSGTLSSQEMPDPSPS | 31 |
| | TPELKVLPNTLKYVFLGPNETLPVIISSKLTMEQECKLTKVLLEHKAAIGWSLSDLSGISPSYCTHRIYTEEEARPSRE | |
| | MQRRLNPNMREVVKNEIIKWLDAGIIYPISDSPWVSPIHVVPKKAGLTIRVNDRGESIPTRTPTSWRVCVDYRKLN | |
| | AATKKDHFPLPFIDQILERLAGNKFFCFLDGFSGYNQVAIDPRDQEKTTFTCPFGTFTFRRMPFGLCNAPATFQRCM | |
| | LAIFHDMVGDFLEVFMDDFSVFGPSFDECLANLTRILRRCIDVNLVLSWEKSHFMVHEGIVLGHIISDRGIEVDRAK | |
| | LDIIADLPVPGSVKQIRSFLGHVGFYRRFIKDFSKISRPLTHLLSTDVPFDMGPDCIQAFTQLRNAVTKAPVLQPPDW | |
| | SLNFELMCDASDLAVGAILGQRRDRHPFVVYYASKTLDEAQRNYTTTEKELLAVVFGLEKFRSYLLGSHVIVFTD | |
| | HAAIRHLMHKKDAKPRLIRWILLLQEFDIEIRDKVGAQNVVADHLSRLDPDHIGLTLPINEVFPDEHLLAINVASGP | |
| | WYADIANYLASGSLPRGWPKNQRERLMAEAKYYFWDNPLLFRIGADQIIRRCIPDSEFPSVLSFCHDQACGGHFSG | |
| | RKTAAKVLQCGFFWPSLHQDAHTYAKHCTRCQSLGSMARRDMMPLQHILAAEIFDIWGIDFMGPFPPSNGFEFIL | |
| | VAVDYVSKWIEA | |
| 2691339 | ENPIKIDFFEPLEIEPLNLIDVNFVEKMDMDIDLSYEQDSLLKQNLKNLRLDHCNKEEKDAIRKLCFDYRDIFYCEQI | 32 |
| | PLSFTSEITHKIKLNDESPIYTKSYRFPEIHKKEVKDQIAKMLDQGIIQHSISPWSSPIWVVPKKLDASGRRKWRLVID | |
| | YRKLNEKSCNDRYPLPNITDILDKLGRANYFTTLDLASGFHQIQVDPDDIPKTAFSTEGGHYEFKRMPFGLKNAPST | |
| | FQRVMDNILRGLHNEICLVYLDDIIIFSTSLDEHIQRLKSVFDRLRKSNFKLQLDKSEFLQKTVQYLGHIITPQGVKP | |
| | NPDKVSTIKRFPIPRTQKDIKSFLGLLGYYRRFIKDFAKITKPLTKCLKKNAKVTHDQNFIDAFNTCKEILVNDPILQ | |
| | YPDFSKPFILTTDASDVALGAILSQGTLPNDRPVAYASRTLNETESKYSTIEKELLAIVWACKHFRPYLYGRKFTIYT | |
| | DHRPLTWLFSLKEPNSKLVRWRLKLEEFEYDIIYKKGKLNTNADCLSRVTLNAIDNESMLNNPGDIDQDILDTLQN | |
| | VTQNLQTLQDFQKPSTSNTNDKINIISDIQIKPPDNSQNDNTNTSTQHSTANETTNDGIKIFDEIINNKTNQILVFPNVI | |
| | YKLDIKRETYENHKIVTVKIPIINNEQMILQFLKENTDPKHVYCMYFQSDELYNDFCTVYLKHFSEKGPKLIRCLKL | |
| | VNTVADKEEQILLIKNHHGSKTNHRGINETLEKLKFNYYWKNMKSTVSNFINACDICQRA | |
| 2691341 | MIAMDDEEWGEEDIREFTKLVENSEKAWEPASEKLEVINLGNKQEKKELKIGTLVTTEERNKL VSLLHEY ADVFA | 33 |
| | WTYADMPGLDTDIVVHKIPLIEGSKPVKQKTRRMRPDILLKVKSEIQKQWDAGFLDVVQYPQWVANVVVVPKKD | |
| | GKIRVCVDYRDLNKASPKDDFPLPHIDILVDNAARNATYSFMDGFSGYNQIRMAEEDKEKTTFVTPWGTFCYKV | |
| | MPFGLKNAGATYQRAMVTLFHDMMHREVEVYVDDILAKSKDEEDHVQVLRRLFERLRKYQLKLNPAKCSFGVK | |
| | TGKLLGFIVSGRGIEVDPDKAKAIQEMPAPKTEKKVRSFLGRLNYIARFISQLTVTCEPIFRLLRKKNPGVWDNDCQ | |
| | EAFDKIKRYLQNPPLLVPPTPGRPLILYLTVTETAMGCVLGQHDESGRKEQAIYYLSKKENDCESRYTTIERLCCAL | |
| | VWSAKRLRQYMLYYTTWLISKLDPLKYICEKPYLSSRIARWQVLLAEYDIVFMTRKAVKGSIIADHLADHAMENY | |
| | EPLNFDLPDEDVIVIENEGGESDQWTLYFDGAVNVSGNGAGAVVISPENKQYPVSTRLLFECTNNTAEYEACIIGLE | |
| | AALELKAKKLEVFGDSLLIICQVKGEWQTKDEKLKLYQNYLLRLANEFEEIKFTHISRDKNQFADALATLASMTQ | |
| | VNAKSKIQPIDIEVRSFQAHCCLIEDSLDGKPWYNDIKKFLQHREYPQGISKTDEKTLRRMTMNFYLDGEILYKRSF | |
| | DGTLLRCLNENEIEQALKEVHEGICAAHANGHTMAKQMQRSGYSG | |
| 2691343 | MDIPVRPVRQRRRKFNEERRLVVKEETQKLLSAGHIREIQYPEWLANVVLVKKANGKWRMCVDFTDLNKASPKD | 34 |
| | SYPLSSIDALVDSASGCEMLSFLDAFSGYNQIKMHPRDEGKTTFMMETCSYCYKVMPFGLKNAGATYQRLMDKV | |
| | LAPMLGRNVQAYVDDMVVASHDRRRHTADLEELFVTISKYRLKLNPKKCVFGVEAGKFLGFMLTKRGIEANPDK | |
| | CAAIIAMRSPTSVKEVQQLTGRMTALSRFVSAGGEKGYHYFQCLKRNSRFAWTDECEAAFVKLKEYLATSPVLCK | |
| | PVASVPLRLYFAVTERAISSVLVQERNQVQRPIYFVIKALQGPETRYQSLEKAALAVVFSARRLRHYFHSFTIVVMI | |
| | NLPIQKVLQKPDVAGRMVRWAVELSEFDIQYEPRGSIKGQVYADFVAELSPGGEQEVESGSQWLLSVDGSSNQQG | |
| | SGAGIVLEGPNGVLIEQALRFAFKASNNQDEYEALIAGMLLAKEMGARNLLVKSDSQLITGKVLGEFQAKDPQMA | |
| | AYLRYVQLLKGAFSALELVHVPREQNARA | |
| 2691345 | MGSFLLVPEADYNLLGRDLMIELGINLEVRNKEIAVKLYALTAEDEARINPEVWYTPESVGKLDITPFEITIMNPEIP | 35 |
| | IRVKQYPMSQEGKKGLQSVIQRLLKQGLLEPCMSPYNTPILPIKKPDGSYRLVQDLREVNKRTLTRFPVVANPYTL | |
| | LSQLSPELKWYSVIDLKDAFWACPLKEECRNYFAFEWEDPETHPKQQFRWTVLPQGFTESSNLFGQALDQVLSTY | |
| | ELNPEVTLVQYVDDLLLAGENQEAVREESIKLLNFLTLKGLKVSKSKLQFTEEEVKYLGHWLSKGTKKLDPERVK | |
| | GILSLQAPKSKRQVRQLLGLLGYCRQWIENYSKKVKFLYQKLVREGLIKWSQTDKKCFEDLKADLVNAPVLSLPD | |
| | TKKPFYLFVNTEEGMAYGVLTQKWADRKKLIGYFSKLLDPVSRGWPTCLQAIVATALLVEEAGKVTLGGELKVY | |
| | TPHNIRGVLQQKAKKWITDARLLKYEGILIHSPKLEIETTSLQNPAQFLYGEPSEELTHDCVRLIESQTKIREDLEEEE | |
| | LPSGEKLFVDGSLRVVNGKRKSVYAIIDGNTSEVKESGPLDTTWSAQACELFAVLKALQLLKGKVGSIFTDSRYAY | |
| | GVVNTFGKIWEERGLINTQGKNLVHETLIRQTLEALRGPRQVAVVHVKGHQKGASVQIRGNNLADEEAKRAALLI | |
| | VKETEKPREATGNSKSFTARGIEK | |
| 2691347 | VGGQSVDFLLDTGATYSVLTEAPGPLSSRSASVMGLSGRAKRYYFSYSLSCNWDSVLFSHEFLIVPESPSPLLGRDI | 36 |
| | LSKVHASVFMNMEPSLSLPLVEQNVNPRVWADGKSVGRAQNAIPVVVKLKDPHVFPHKKQYPLKPEVKEGLKPII | |
| | ENLKEQGLLIPCNSPCNTPILGIKKSNGKWRLVQDLQIINEAVVPLHPVVPNPYTLLSEIPERAKYFSVIDLKDAFYS | |
| | VPLAEESQFLFAFEDPTQPASQLTWTVLPQGFRDSPHLFGQSLSRDLQNFNSSEAVVLQYVDDILLCAETEEACSRA | |
| | SEDFLNFLAGCGYKASREKAQLCQQSVRYLGLIISEGTRAIGPERIKPILNHPLPMTLRQLRGFLGITGYCRIWIPGY | |
| | GELARPLYKLIAETQQAQTDKLVWSPETQKAFKVLQTALLQAPALSLPTGSEFNLFVTERKGVALGVLTQPRGPH | |
| | QQPIAYLSRELDVVSRGWPHCLRVIGAAALLAPEALKIINGRNLTVLTSHDVSGILNSKVNIWMTDSRLLKYQSLL | |
| | LEGPVTKLKVCGNLNPATFLPEKENETPDHDCSQFLTLNYAAREDLKDTPLDNPDMEIFTDGSSFVRDGKRKAGY | |
| | AVVTAEQVLEAKSLPQGTSAQLAELVALTRALELSKGQRMGLIIYVSLLLLTPKILSLPLDPQDNVFLSWAHSYAA | |
| | FHNRSNCWVCGALPSSSVEGFPWWTSPLQGKDFLQVCEYLRQQSHAMPLLHLMTSTNPKMDWCNTL | |
| 2691349 | MLSEEALEVLKTVPPCVWSQGKCDVGRMVNAKPVHVRPKTNYRPNIRQYPLKEDAKVGIKPVIEEMLKAGILREC | 37 |
| | PDASCNTPIFPVKKADGQSWRMIQDLRAVNDAVQSTAPTVPDPHTLMNGLKPNMAWFSVIDLSNAFFSIPVEKDS | |
| | QGWFGFTHEGTKYTYTRLPQGYVDSPTIFSREIANCLATLQIPDTSQILVYVDDILVASPTQEESTKVTLQVLTHLA | |
| | KTGNKVSRQKLQFVKEEVIFLGHNLNREGRRIHSERKQTVLNEPKPTTKKQMMQFLGLTNYCRAWIPDYAGMTQ | |
| | PLLDCIYSDNDMKMSGKIEWTQEAEAAFESTKQCLVGSSVLALPDYEKPFVQTVDCKKGFMTSVLAQKHGTKLR | |
| | PVAYYSKRLDPVAQALPVCVQAVCAAAMAVHCTAEIVLFHPLTLMVPHAVTMLLHDTKMAFLSPARYLALTAT | |
| | LMSQPHIVIKRCNILNPATLIPTAEDGEPHCCKEETDRTCKPRPDLKDIQLLSGETWFVDGSCSKSITGQNQTGFAV | |
| | VSHSQVIKAGRLPHTYSAQAAELVALTEACKAGVGKDVTMWTDSQYAHSTVHIFAAQWARRGMKTSTGKPVTH | |
| | AQLLTDLLKAVLLPKSIAICKCAAHTSGKDAVTLGNAHADKVAKLAAMGEYGFHILLQKGESVSQPIPL VILRDM | |
| | QNSAPDREKKKWLTDGATTDPEGTFRINNKIVLPVSLYKTAAHLSHGPCHVSTGGMVTIINEHFHTYNYITFSKNF | |
| | CRACVVCCRHNAQGNERPQRGK | |
| 2691351 | MVMALLAATSAPTVPTVPVELSDISETLWTTISTETGLLKSAEPVYITYHTDIPLPRKMQYPLPKEAIEGIQPVLQQL | 38 |
| | LTQGIIKEATSPCNTPILPVKKPKPGSYGKPVYRFVHDLRLINAIVIPRAPVIPNPATVLTAIPPEATYFTVIDLSSAFFS | |
| | NPVHPDSQFLFAFTFDGRQYIWTRLPQGYRESPTIFSQYLKWDLDSIRCAMGSTLIQYVDDLLLCSTAKDACVQDT | |
| | RILLFALAEKGHKVVLSKLQFAQSEVEYLWHRISYVSTALTQDRIESVLSKPRPTTKKQLHQLLGASGYCRAWIPA | |
| | YVELVHPLTDLLLDSIPEPLPWTTLHDNALTALKQALTSAPALGRPDYAKPFTLFCHECYGTASGVLTQPFGPGQR | |
| | MVAYFSVLLDPCAQGLPPCLRAVGAVAMLVETALGLTLGHPLLVQVPHAVAALLNRGSTQHLTAARLTKYELAL | |
| | LANQTITLHRCEVLNPATLLPLPEDGMPHNCLHLMDLISTPRPDLSDVPLQNPDLQLFCDGSAHQDEFGALRVGYA | |
| | IVILMTTLEAHSLPTQKSAQAAELVALVRACQLAENKTVAIYTDSKYAFSVCHSTGQLWKHHGFLTSCGSQISHAA | |
| | LISALLEAIQLPAAVSVTHCDFVCVKIHRRKHCLEPWWSAPAQVLLTTPTAVKIEGIDRWVHCSHCKKVPAPTSPT | |
| | TAITTDALT | |
| 2691353 | VFAWDAYDAPGVDPSLICHHLNVNPSSTPKKQPPRRPSKEHASAVKDEVAKLKRAGAIKEVFYPEWLANTVVVK | 39 |
| | KKTGKWRVCVDFTDLNKACPKDPFPLPRIDRLVDATVGHPRMSFLDAFQGYHQIPLALEDQEKTAFMTPIGNYHY | |
| | KVMPFGLKNAGSTYQRMMTRMFEPQLGKIIEVYIDDMVVKSRRASEHVKDLGTIFTILREHRLRLNASKCSFGVG | |
| | SGKFLGYMVTHRGIEVSPDQIKAINSLQAPRNPKEVQKLTGMIAALNRFISRSADRCRPFYLLINKWKEFAWSEDC | |
| | VQAFQQLKDYLSRPPIMSSPEADEVLFAYIAVAPHAVSLVLIRDDNGVQRPVYYVSKSLHEAEVRYLPLEKAILAIV | |
| | HATRKLPHYFQAHTVIVLTQLPLRAVLRSADYTGRIAMWSALLGAFDIKYMPRSSIKGQVLADLVAEFAEPSIETIT | |
| | EKRDMDGKSVGAISTGRNTHWKVYVDGAANQRGSGVGIVLISPDGAAIEKSLRLGFSATNNEAEYEALLQGMTM | |
| | VQRLGGKMIEAFSDSRLVVGQVMGELEARDTRMQEYLGQVKRLQTNFESFNLTHISRSVNTHADSLATLATSSAH | |
| | NLPRVILVEDLAQASPISRGPARVHQIRKSHSWMDPIKNFLQNDVLPEENWKPRRYEGMLLGFGCQRTTNYIGAPI | |
| | LDRTYSAYTQKNPNLCLRSYTRGFVEVIPEGDRWHTGHLPRGTGGRTCRERPKNTLRSAISAKDSPRISTNPEEFST | |
| | PSRVHGRSHNGALI | |
| 2691355 | VLIRDTSITTNPLPARYHDFSDVFEKKNADRLPEHRLYDCPIDLQEGAHPPFGPIYGLAEPELEALREYLKENLAKG | 40 |
| | FIQPSKSPAGAPILFVKKKDGSLRLCVDYRGLNRVTVRNRYPLPLIPELLDRLRTGRVFSKIDLRGAYNLVRIKPGD | |
| | EWKTAFRTRYGHFEYKVMPFGLTNAPAVFQHMMNHIFREYLDHFVVIYLDDILVFSPSMEEHTRHVRLILTKLRE | |
| | HGLYAKSEKCEFDRTSVEFLGYVISPAGITMDPRKVATIHNWPIPTRLKEVQSFLGFANFYRRFIDRFSTIVQPLISLT | |
| | RKDVPFVWTATTQHAFDALKQAFMSAPVLVHPNPAKPFQVETDASDFALGAILSQLDDDGTLHPVAYYSRKFLA | |
| | SEINYPVYDKELAAIISAFAEWRPYLVGAQHRIQVVTDHKNLLYFASSRTLNRRQARWSIFLADYDFEIIFRPGIQH | |
| | GKADALSRRPDLALCPGDDAYTQQSCSLLKPDQLQLSATFMLHDDSLLQEIAQATKTDTFATEILKRLQDSSPATK | |
| | RADLHHFTTHDGLLYRNHLLYVPEGPCRTQVLQTCHDDPLAGHFGVAKTLELVSRGFWWPQPWKLVKEFVKTC | |
| | DVCARSKAVHHRPYGLLHPLPIPNRPWASISMDFITDLPPVNGVDTV | |
| 2691357 | VSAPDPPVPHADKIIWETQIPVWVDQWPLTQEKLKAASMLVQEQLAADHVEPSTSPWNTPIFVIKKKSGKWRLLQ | 41 |
| | DLRAINRVMRPMGALQPGIPTPVAIPKDYYKMVIDLKDCFFTIPLHPEDRPYFAFSLPRINFQGPMQRFQWKVLPQ | |
| | GMANSPSLCQKYVAQAVDPVRKRWPQIYILHYMDDLLIAAPDTHNLNGCYLDLYKALTTLGLQIAPDKVQTQDP | |
| | YTYLGLQLQGNQIQTPKIQLRVDHLHTLNDFQKLMGDIQWLRPYLKLPTCVLLPLNDILKGDSHPTSPRKLTPEAQ | |
| | QALNQVTKAITQQFVTQISYNLPLILVILHTPHTPTGIFWQRDPHFKNKGCPLFWVHLPTTSSKVITVYPAQVASVIV | |
| | KGRLLSRQHFGKDPDKILIPYTKDQTQFLLQTEDEWSIALSGFLGTIDNHLPSDPLLHFAQLHPFIFPKITQKAPIPQA | |
| | LTVFTDGSSNGCAVFVVGNQPTTFETAYQSAQLVELFAIAQVFITFLDKPVNIYTDSAYIAHSVPLLEISPAIKASSN | |
| | AAFLFHQLQQLIQTRRHPFFLGHIRAHSDLPGPLTQGNAQADAATRSIFPIFAGSIQRATELHTLHHLNSQSLRLFCK | |
| | ITRQQAREIVKACPACT | |
| 2691359 | VSAPDPPVPHADKIIWETQIPVWVDQWPLTQEKLKAASMLVQEQLAADHVEPSTSPWNTPIFVIKKKSGKWRLLQ | 42 |
| | DLRAINRVMRPMGALQPGIPTPVAIPKDYYKMVIDLKDCFFTIPLHPEDRPYFAFSLPRINFQGPMQRFQWKVLPQ | |
| | GMANSPSLCQKYVAQAVDPVRKRWPQIYILHYMDDLLIATPDTHNLNGCYLDLYKALTTLGLQIAPDKVQTQDP | |
| | YTYLGLQLQGNQIQTPKIQLRVDHLHTLNDFQKLMGDIQWLRPYLKLPTCVLLPLNDILKGDSHPTSPRKLTPEAQ | |
| | QALNQVTKAITQQFVTQISYNLPLILVILHTPHTPTGIFWQRDPHFKNKGCPLFWVHLPTTSSKVITVYPAQVASVIV | |
| | KGRLLSRQHFGKDPDKILIPYTKDQTQFLLQTEDEWSIALSGFLGTIDNHLPSDPLLHFAQLHPFIFPKITQKAPIPQA | |
| | LTVFTDGSSNGCAVFVVGNQPTTFETAYQSAQLVELFAIAQVFITFLDKPVNIYTDSAYIAHSVPLLEISPAIKASSN | |
| | AAFLFHQLQQLIQTRRHPFFLGHIRAHSDLPGPLTQGNAQAD | |
| 2691361 | VEEAAPIQGNNRDQQAANAMEREIGGKTFKLGRLLSQEKQEGVAEVISRHLDAFAWSASDMPGIDPDFLCHHLSM | 43 |
| | DVAVRPVRQRRRKYNEERQLVVKEETQKLLSAGHIRVIQYPEWLANVVLVKKANGKWRMCVDFTDLNKACPKD | |
| | SCPLPSIDALVDSASSCKVLSFLDAFSGYNQIKMHPRDESKTAFMIETCSYCYKVMPFGLKNTGTTYQRLMDKVLA | |
| | PMLGRNVYAYVDDMVVASQDREHHVADLEELFVTISKYRLKLNPEKCVFGVDAGKFLGFMLTERGKEANPNKC | |
| | AVIIAMRSPTSVKEVQQLTGRMAALSRFVSAGGQKGHPYFQCLKRNSRFAWTDECEAAFIKLKEYLATPPVLCKS | |
| | VTGVPLRLYFTVTERAISSVLVQEQDHSQKPISFVSKALQGAETRYQALEKAALAVVFSARRLRHYFHSFTVVVIA | |
| | NLPIQKVLQKPDVAGRMVRWAVELSEFDIQYEPRGSIKGQVYADFVAELSPGGEQEVEVGSQWLLSVDGSSNQQ | |
| | GSGAGIVLEGPNDVLIEQALRFAFKASNNQAEDEALIAGMLLAKEMVAQNLLVKSDSQLITGQVSGEFQAKDPQM | |
| | AAYLKYVQLLKG | |
| 2691363 | VYRKQFKIPEAHQTFIEQTLDEWLKLGVVKRSNSLYNSPLFCVPKKQGRGLRIVQDFRELNNHSHIDKYSMKEITE | 44 |
| | CIGDIGRANSSIFSTLDLTSGFWQMKLDEASQPLTAFTIPGQGQFQWITSPMGLLGCPASFQRLMETVLRGIKNVLV | |
| | YIDDLLIHTATHEEHLIVLEQVFERLHKNHLKINLEKCVFGNPEVSYLGYTLTPNGIKPGRNKLQAIKDAQPPTNIK | |
| | MVRSFVGLCNFFRTHIKDFAIIAAPLFKVTRKDSNYKSGQLPPDALHAFRVLQLQLTSEPVMAYPRSDRQYALITD | |
| | AATGTAETPGGLGAILTQIDKSGNHFAISFASRQLKDHEKNYSPFLLEAAAAVWGMDVFNEYLRGKQFILFTDHKP | |
| | LEKLGHLHTKTLNRLQTALLEHDFVIQYKKGAIMPADYLSRLPTLQVQNVEATVAAFDPFQPDLSKLQVPFQVNT | |
| | AADVIAAFDPFQPNLKELQFQDTDLQAIYLFLKNGQWLPHLTKRQINSLSALSQKVFFDKNKLAWIRLDDYKYPR | |
| | TALWLPEFYRKEALCESHNQIFAGHNAAQKTYLKLTSSYFWPNVYSHVLQHTQTCFRCQQRKSSRAKQPPLAPLPI | |
| | PDLPNVRIHADLFGPMLGVEKKFAYVLCITDAFTKYAMVTKVDNKDAETVARAIFDNWFCKFGIPAQIHTDGGKE | |
| | FVNKLSAELFELLNVQHSKTSPYHPQCNAQVEVFNKTVKKYLASYVDKTTLNWNDFLPALMLAYNTSYHSTIATT | |
| | PFELLFGVK | |
| 2691365 | MKEDHRKIEELVPRKFLKWRKVFGKVESERMPTRKVWDHAIDLKEMFKPQKGRIYPLSKNKREEVQNFVEDQLR | 45 |
| | KGYIRPSKSPQTLPVFFVGKKDGSKWMVMDYHNLNNQTVKNNYLLPLITELIDNMGSKKVFTKMDLRWGFNNM | |
| | RIKEGDEWKGVFTMHIGSFEPIVMFFGMTNLPATFQAMINEILRDLINKGKVAAFVDDVLVGTETEKGHDKIVEEI | |
| | LRRLEENDLYVKPEKCIWKVWKIGFLGVVMEPNGIEMEEEKVDRVLSWPELENVKDIRKFLGLANYYRRFIKDFA | |
| | RVARPINMLMRKDVKWQWGVEQQKAFDKLKRVFTTKLVLAAPDLDKEFRVEADVSNYATRGVLSMKCSDEM | |
| | WRPIAFISKSLSDTERNYEIHDKKMLAVVRCLEAWRYFLEGATTKFEIWTDHKNLEYFMKAQKLNRRQARWALY | |
| | LSRFNFMLKHVLGSKMGKADSLSRRPDWEVGVEKDNKDQKLVKPEWLEVRKTEMVEIIVDGVDLLEEVRKSKV | |
| | RDDEVVKVVEEMKKAGVKILRDEEWREADGVMYKKEKVYVPKDNKLRAEIIRLHHDMPVGGHGRQWKMVEL | |
| | VTQNFWWPGITKEVKRYVEGCDACQHNKNRTEQPAGKLMPNSIPEKPWTHISVDFITKLPLVQGYDSILVVVDWL | |
| | TKMVHFIPTMEKTSAEGLARLFRDNVWKLHGLLES | |
| 2691367 | MKEDHRKIEELVPRKFLKWRKVFGKVESERMPTRKVWDHAIDLKEMFKPQKGRIYPLSKNKREEVQNFVEDQLR | 46 |
| | KGYIRPSKSPQTLPVFFVGKKDGSKWMVMDYHNLNNQTVKNNYLLPLITELIDNMGSKKVFTKMDLRWGFNNM | |
| | RIKEGDEWKGVFTMHIGSFEPIVMFFGMTNLPATFQAMINEILRDLINKGKVAAFVDDVLVGTETEKGHDKIVEEI | |
| | LRRLEENDLYVKPEKCIWKVWKIGFLGVVMEPNGIEMEEEKVDRVLSWPELENVKDIRKFLGLANYYRRFIKDFA | |
| | RVARPINMLMRKDVKWQWGVEQQKAFDKLKRVFTTKLVLAAPDLDKEFRVEADVSNYATRGVLSMKCSDEM | |
| | WRPIAFISKSLSDTERNYEIHDKKMLAVVRCLEAWRYFLEGATTKFEIWTDHKNLEYFMKAQKLNRRQARWALY | |
| | LSRFNFMLKHVLGSKMGKADSLSRRPDWEVGVEKDNKDQKLVKPEWLEVRKTEMVEIIVDGVDLLEEVRKSKV | |
| | RDDEVVKVVEEMKKAGVKILRDEEWREADGVMYKKEKVYVPKDNKLRAEIIRLHHDMPVGGHGRQWKMVEL | |
| | VTQNFWWPGITKEVKRYVEGCDACQHNKNRTEQPAGKLMPNSIPEKPWTHISVDFITKLPLVQGYDSILVVVDWL | |
| | TKMVHFIPTMEKTSAEGLARLFRDNVWKLHGLLESIISDRGPQFAAGLIRELNGILGIASKLLTVFHPQTNGQTERV | |
| | NQELEQYLRMFINYRQEQWPEWLGTAEFAYNNKVHSST | |
| 2691369 | MVKIRGDLSSEMKQQLYNPLKEFKDIFAWSYKDMPGLDFEIVQHRLPLKPECHPIKQRLRRMKPEVSLKIKEEVEK | 47 |
| | QFNAGFLAVAQYPQWVANVVPVPKKDGSVRMCVDYRDLNRASPKDDFPLPHIDTLVDNTATSSLYSFMDGFSGY | |
| | NQIKMTPEDMEKITFITLWGTFCYKVMSFGLKNAGATYQRAMMALFHDMMHKEIEVYVDDMIAKSESEEEHLVN | |
| | LKKLFERLRKYKLRLNPSKCTFGVKSGKLLGFIVSQKGIEVDPDKVRAILEMPHPCTEKQVRGFLGRLNYIARFISQ | |
| | LTATCEPIFKLLRKNQVVKWNENCQNAFDKIKEYLKEPPILHPPVPGRPLILYLTVLDGSMGCVLGQHDGTGKKEH | |
| | VIYYLSKKFTDCEQRYSSLEKTCCVLAWTAHRLRQYMLSHTTWLVSKMDPIKYIFEKPALTGRIARWQMLLSEFNI | |
| | VYVIQKAIKGSALAEYLAQQPIDDYQPMQPEFPDEDIMTLFVSEDDGNERKWVLFFDGSSNALGHGIGAVLISPEK | |
| | QYIPMTARLCFDCTNNIAEYEACAMGIRAAIEYKARRLNVYGDSALVIHQVKGEWETRDQKLIPYKAYIRGLVEY | |
| | FDEIEFHHISREDNQLADALATLSSMFAISQEEELPMIKMQSHENPVYCNFIEEEVDGKPWYFDIKRYLQNREYPDS | |
| | ASENDKRMLRRLASSFILDGDVLYK | |
| 2691371 | MVKIRGDLSSEMKQQLYNPLKEFKDIFAWSYKDMPGLDFEIVQHRLPLKPECHPIKQRLRRMKPEVSLKIKEEVEK | 48 |
| | QFNAGFLAVAQYPQWVANVVPVPKKDGSVRMCVDYRDLNRASPKDDFPLPHIDTLVDNTATSSLYSFMDGFSGY | |
| | NQIKMTPEDMEKITFITLWGTFCYKVMSFGLKNAGATYQRAMMALFHDMMHKEIEVYVDDMIAKSESEEEHLVN | |
| | LKKLFERLRKYKLRLNPSKCTFGVKSGKLLGFIVSQKGIEVDPDKVRAILEMPHPCTEKQVRGFLGRLNYIARFISQ | |
| | LTATCEPIFKLLRKNQVVKWNENCQNAFDKIKEYLKEPPILHPPVPGRPLILYLTVLDGSMGCVLGQHDGTGKKEH | |
| | VIYYLSKKFTDCEQRYSSLEKTCCVLAWTAHRLRQYMLSHTTWLVSKMDPIKYIFEKPALTGRIARWQMLLSEFNI | |
| | VYVIQKAIKGSALAEYLAQQPIDDYQPMQPEFPDEDIMTLFVSEDDGNERKWILFFDGSSNALGHGIGAVLISPEKQ | |
| | YIPMTARLCFDCTNNIAEYEACAMGIRAAIEYKARRLNVYGDSALVIHQVKGEWETRDQKLIPYKAYIRGLVEYF | |
| | DEIEFHHISREDNQLADALATLSSMFAISQEEELPMIKMQSHENPVYCNFIEEEVDGKPWYFDIKRYLQNREYPDSA | |
| | SENDKRMLRRLASSFILDGDVLYK | |
| 2691373 | MKYPYSNVYSITCYDQIDKCVQQVCDFDCEDGLSITLSYGYDFTKIEEMERHVCVPQNVHESVLALQALQTVPHG | 49 |
| | NLFVDLVLSHKKLLPSILQAPELELKPLPDNLKYVFIGDNNTLPVIIAKGLTNIQEEKLVKLLCDHKTAIGWTLADIK | |
| | GISPSMCMHHILLEDNAKPTREMQRRLNPPMMEVVKAEILKLLDAGVIYPITDSKWVAPIHVVPKKTGITLVKNKN | |
| | DELIPTRISSGWRMCVDYRKLNLSTRKDHFPLPFMDQMLERLAGKSFYCFLDGYSGYNQIVINPEDQEKTTFTCPF | |
| | GTYAYRRMPFGLCNAPATFQRCMMSIFSDYVERIIEVFMDDFTVYGDSFDKCLENLSLILKRCIETNLVLNYEKCY | |
| | FMVEQGIVLGHVVSSRGLEVDKAKIDVISSLPYPSCVREVRSFLGHAGFYRRFIKDFSKITAPLCRLLAKEVDFVFD | |
| | QACKDAHDELKRRVTSAPIIQPPNWDEPFEIMCDASDYAVGAVLGQRIGKNLHVIAYASRMLDGAQCNYHTTEKE | |
| | LFAVVFALEKFRSYLLGTKVIVFTDHAALRYLLKKKESKPRLIRWILLLQEFDLEIKDKKGAENHVADHLSRLRTE | |
| | DIQIETIRETFPDEQLYMLHSSTRPWYADLVNYLVTKEFPPGLSKPQKDKLRADAKYYFWDDPYLWKFCADQVVR | |
| | RCVP | |
| 2691375 | MPGVPRNLIEHSLNVSKTARPIKQKLRRFARDKKEAIRAEITRLLAAGFIKEVYHPDWLANPVLVRKKNNEWRMC | 50 |
| | VDYTDLNKHCPKDPFGLPRIDEVVDSTAGCELLSFLDCYSGYHQISLKEEDQIKTSFITPFGAYCYTTMSFGLKNAG | |
| | ATYQRAIQQCLHDEIRDDLVEAYVDDVVVKTRDASTLIDNLDRTFKALNRYKWKLNPKKCIFGVPSGLLLGNVVS | |
| | RDGIRPNPSKVKAVLDMRPPKNVKDIQKLTGCMAALSRFISRLGEKGLPFFKLLKASEKFSWTEEADTAFNQLKTF | |
| | LTSPPVLTAPQPNEDLLLYIAATDRVVSTAIVVERDEPGHVFKVQRPVYFISEVLNESKARYPQIQKLIYAILITSRKL | |
| | KHYFDGHRVLVTTSFPLGDILRNKDANGRIVKWAMELCPFSLEFQSRTTVKSQALVDFIAEWTDLNEPSVPDVSD | |
| | HWSMFFDGSLNINGAGAGILFVSPNKDKLRYVLRILFPASNNVAEYEACLHGIRLAVELGVKRLYVYGDS | |
| 2691377 | VPGNEDLDFRKQISPELDEEETEKLVKALGRFVDVFAKGNEPLGMCNMAEHEIPLVPNAKPVYQSLGSGAYKEQL | 51 |
| | IHRELMSKMKARGAIEVSVGPWGARVLLAQKKDGTWRFCVDYRLLNALTVNSVYPMPSIDGTLARLHGAKIFSI | |
| | MDLECGYWQIPIKKEDRVKTAFITADGVFQFICMPFGLCTAPSTFQRTMDLVLGGLRWSVCLVYLDDIIVYARNTE | |
| | EHRQRLVMVLSALRKANLKLKLAKCRFAEKEITALGHRISAEGVRPDPDKVRAVKEFPGLPTSGKRADLVKWVK | |
| | SFLGLVSYYRRFFPGFAEVAKPLMDLTKDKTLFIWGASHQKSFDELKERLANAAVQAYPDYSLEFEIHPDACGYG | |
| | VGAVLVQRQMGEERPLAFASRIMTASERNYSITEKECLALVWAVKKFRFFIWGRPIRIVTDHHALCWLQSKKDLA | |
| | GRLSRWAMQLMEYKYTIAHKDGRLHADADALSRYPLALGLGSDATDKESRDLVVNVVTQCDRSELQEGQRSEW | |
| | AYVFNNHEQEKETANYTIRNGLLFRIRLWMASRGKLRCDSACRRPCGRGY | |
| 2691379 | MSLMEEVVSRENMQAAYRRVVRNRGAAGPDGMTVEDLADHCRQHWPRIREELLAGRYRPQPVRRVTIPKPGGG | 52 |
| | ERALGIPTVLDRMIQQALLQVLQPVFDPRFSPDSYGFRPGRGAHDAVLRARDHVRGGRRWVVDLDLEKFFDRVN | |
| | HDVLMSRVARRIEDKGVLRLIRRFLQAGVLEGGVASPRLEGTPQGGPLSPLLSNILLDEWDKELERRGHRFVRYAD | |
| | DCQIYVQSQRAGERVMRSMTRFLEEVLRLRVNRVKSAVARPWERQFLGYGFTQH | |
| 2691381 | MEIETPQRLRKLQQVLHRNAKANSKWRAWSLYADLLREDVLAHAAAKVIANKGAAGIDGTSVDDIRSSESRERF | 53 |
| | LSDLREELSSKAYKPSPVLCVRIPKKDGKTRALGIPTVKDRVVQPALVLLLEPIFEADMHAESYGYRKGKNAHQA | |
| | MDSICRALYSGRHEVIDADISGYFDNIDHAMLMKLVKRRVSDGSILKLIKSFLTAPVVENGTGRKNDKGTPQGGVL | |
| | SPLLANLYLNGLDHQVNGKAGQGAQMVRYADDLVILCHRGKGAELLERLDRYLATKGLSLNRRKTRRVDFTKE | |
| 2691383 | LGIPTLTDRLIQQALHQVLSPIFEAEFSESSYGFRPGRNAHQAVKAAKQYVAEGRRFVVDMDLEKFFDRVNHDVL | 54 |
| | MAKVAKKVEDGRVLKLIRRYLEAGMMAEGVVSPRTQGTPQGGPLSPLLSNILLTDLDRELERRGLAFCRYADDC | |
| | NIYVRSQAAGERVLAGISRFLSERLKLRVNEAKSAVARPWERKFLGYSLTWHKTPKLRIAPASLQRLKGKIRKML | |
| | KGASGRSVRRTIEELNPVLRGWAAYFKLAETKSALEELDGWLRRKLRCILWRQWKRPYTRARNLMKAGLKEER | |
| | AFRSAFNQRGPWWNSGASHMNAAFRNVYFDRLGLVSLLDTTRRLQYLS | |
| 2691385 | EYKVSIAPIPEYILGIDIMSGLTLQTTVGEFRLRERCISIRAVQAIIRGHAKIEPIFLPQPRCITNTKQYRLSGGEEEFTKI | 55 |
| | VQELERVGIIRPAHSPYNSPIWPVRKPDGTWRMTVDYRELNKVTPPIHAAVPNIASLMDTLSREIETYHCVLDLAN | |
| | AFFSIPIAKESQDQFAFTWEGRQWTFQVLPQGYVHSPTFCHNLVASDLANWNKPSTVKMFHYIDDLMLTSDSIEAL | |
| | EKTVPSLITYLQEKGWAINPQKVQGPGLSVKFLGVVWSGKTKVLPSAIIDKIQAFPVPTKPKQLQEFLGILGYWRSF | |
| | IPHLAQLLKPLYRLTKKGQVWDWGRTEQEAFQQAKIAVKQAQALGIFDPTLPAELDVHVTQEGFGWGLWQRQG | |
| | SVRIPIGFWSQIWHGAEERYSMVEKQLLATYSALQAVEPITQTAEVIVKTTLPIQGWVKDLTHIPKTGVAQSQTVA | |
| | RWVAYLSQRSRLSSSPLKEELQKILGPVTYHSETPEEIVVTCPEESPVQEGKYPIPEDAWYTDGSSRGNPSRWRAVA | |
| | YHPSTETIWFEEGDGQSSQWAELRAVWMVITQEPGNSALNICTDSWAVYRGLTLWIAQWATQDWTIHARPIWGK | |
| | DMWVDIWNVVRHRTVRAYHVSGHQPLQSPGNDEADTLARV | |
| 2691387 | MIDGLGFANSFQNPSHNSLIFISTYVNKRIMNIMVDTGSQHSFINKNCLREFDKLQSFEISPQNFFMADGMTTFQVK | 56 |
| | DTIRLNISIGTFDTTAVAFVTTHLCTDMILGMDYLSQYDIEIKTKQNYITFNFGNEQVILSTNLESSSKRHSSSNPELIR | |
| | SSINQQFHTSALSSINNLEQIISNLANHMTNPTQQNNLKSLLFKFQSTEDTSKYTIAKTEIFHVIETETHIPPASKCYPG | |
| | NPTLNAELRKIVDKLLDAGLIANSQSPYAAPALLVKKKDNTWRLVIDYKKLNSITLKDNYPLPNMEMALQTLGCG | |
| | YSYFSKFDLRSGFWQLPIDPKDRFKTAFATPFGLFEWLVLPQGLRNSPPTFQRTMNRVLSACTEFSLVYLDDIVIFS | |
| | HTFDDHLVHIEKVLSALREHNLTLAPSKCELAKQSIEYLGHVISSKTIAPLPDRIKSIILLPEPKTLTQANKFIGSLSW | |
| | YRKFIPAFATLAAPIHSITNLSKSNRHKFHWGIDQSKSFAALKQFLTSSPIFLDFPVDGQPIFLATDASLVGLGGVLY | |
| | QEVQGEKRILYYHSELLTPAQKRYHPLELEALAIFKCVNRMKSFLLGREIFVYTDNCPICHMLDKKVSNKRVEKISI | |
| | LLQEFNIRRIIHVKGEYNCLPDYLSRHPIEQDNEFLNSNYGLEFVRDDSSSIQIAGAVVTRSKAKALPPPPTNSSQTNS | |
| | QQTTSLISPTSNRDSLNELEPEPESVDMNFDLTQIKQHQLGDVRIQQVIEDLKTNPNSSF | |
| 18266 | MSELLEKILSKDNMNTAYKRVCANKGAGGVDDVTVEELGAYVKENWESIREQIRRREYTPQPVRRVEIPKPDGGE | 57 |
| | RKLGIPTVMDRVIQQGIVQIISPMCEPLFSKWSYGFRPNRSCEMAIRQLLEYLNEGYEWIVDIDLEKFFDNVPQDRL | |
| | MSLVHDIIKDGDTESLIRKYLKAGAMTPQGYKETKLGTPQGGNLSPLLSNIMLNELDKELEARGLRFTRYADDCVI | |
| | AVKSESSAKRVMRTVTDWIQRKLGLKVNMTKSQITRPQKLKYLGFGFHKDSKAKEWKCRPHQESVKKFKVRLK | |
| | ELTCRKKPGTVTSKIAKINQVTRGWINYYALGSMKTAMTEIDAHLRTRLRVIIWKQWKVPKKRQWGLQKLGVSK | |
| | DLARLTAYCGDSYYWVATKTCVVRAISKEVFAKSGLVSCLDYYNERHALKLC | |
| 18268 | MSELLERILSNDNMNAAYKRVCANKGAGGVDGVTVEELGDYIKENWSSIREQIRRRQYKPQPVRRVEIPKPNGGV | 58 |
| | RNLGIPTVMDRVIQQGIVQVLSPMCEPLFSERSYGFRPNRSCEMAIRQLLVYLNEGCEWIVDIDLEKFFDNVPQDRL | |
| | MSLVHDIINDGDTESLIRKYLKAGVMTPQGYEETKLGTPQGGNLSPLLSNIMLNELDKELEKRGLRFTRYADDCVI | |
| | AVKSESSAKRVMRTVADWIQRKLGLKVNMTKTHITRPTKLKYLGFGFYRDNKAKEWKCRPHQDSVAKFKRKLK | |
| | ELTCRKTPGTVTGRIAKINQVTRGWINYYAIGSMKTTMIEIDAHLRTRLRVIIWKQWKVPKKRQWGLQKLGIGKD | |
| | LARLTSYYGDRYQWIVTKTCVARAISKEVLAKAGLVSCLDYYNERHALKLC | |
| 21531 | MSELLEKILSKDNMNAAYKRVCANKGAGGVDDVTVEELGAYVKENWESIREQIRRRKYTPQPVRRVEIPKPDGG | 59 |
| | ERKLGIPTVMDRVIQQGIVQIISPMCEPLFSKWSYGFRPNRSCEMAIRQLLEYLNEGYEWIVDIDLEKFFDNVPQDR | |
| | LMSLVHDIIKDGDTESLIRKYLKAGVMTPQGYKETKLGTPQGGNLSPLLSNIMLNELDKELEARGLRFTRYADDC | |
| | VIAVKSESSAKRVMRTVTDWIQRKLGLKVNMTKSQITRPQKLKYLGFGFHKDSKAKEWKCRPHQESVKKFKVRL | |
| | KELTCRKKPGTVTSKIGKINQVTRGWINYYALGSMKTAMTEIDAHLRTRLRVIIWKQWKVPKKRQWGLEKLGVS | |
| | KELARRTAYCGDSYYWVATKTCVVRAISKEVFAKSGLVSCLDYYNERHALKLC | |
| 21532 | MSELLEKILSKDNMNAAYKRVCANKGAGGIDDVTIEELGDYIKENWESIREQIRHREYKPQPVRRVEIPKPNGGVR | 60 |
| | ELGIPTVMDRVIQQGIVQIISPMCEPLFSEWSYGFRPNRSCEMAVRQLLVYLNEGYEWIVDIDLEKFFDNVPQDRL | |
| | MSLVHDIIKDGDTESLIRKYLKAGAMTPQGYEETNLGTPQGGNLSPLLSNIILNELDKELEGRGLRFTRYADDCVIA | |
| | VRSESSAKRVMRTVADWIQRKLGLKVNMTKTRIARPQKLKYLGFGFYKDSKAKEWKCRPHQESVKKLKDRLKE | |
| | LTCRKKPGTVTSKIAEINQVTRGWINYYALGSMKTAMTEIDAHLRTRLRIIIWKQWKVPKKRQWGLQKLGIGKDT | |
| | ARLTAYCGDRYYWVATKTCVVRAISKDVFAKAGLVSCLDYYNERHALKLC | |
| 21533 | MSELLEKILSKENMNAAYKRVCANKGAGGVDGVTVEELGDYIKEHWRSIREQIRQRQYKPQPVRRVEIPKQNGG | 61 |
| | VRKLGIPTVMDRVIQQGIVQVINPMCEPLFSEWSYGFRPNRSCEMAIRQLLIYLNEGYEWIVDIDLEKFFDNVPQDR | |
| | LMSLVHVIINDGDTESLIRKYLKAGVMTPQGYEETRLGTPQGGNLSPLLSNIMLNELDKELEARGLRFTRYADDCV | |
| | IALKSESSAKRVMRTVSDWIQRKLGLKVNMTKTHITKPLKLKYLGFGFYKDSKTKEWKCRAHQDSIVKLKRKLK | |
| | ELTCRKMPGTVREKIEKINQVTRGWINYYALGSMKTAMTAIDAHLRTRLRVIIWKQWKVPKRRQWGLQKLGVG | |
| | KDLARLTSYCGDRYQWVVTKTCVVRAISKEVLTKAGLISCLEYYNKRHALKLC | |
| 21534 | MSELLEKILSKENMNTAYKRVCANKGAGGVDGVTVEELGDYIKEHWRSIGEQIRQRQYKPQPVRRVEIPKQNGG | 62 |
| | VRKLGIPTVMDRVIQQGIVQVINPMCEPLFSEWSYGFRPNRSCEMAIRQLLIYLNEGYEWIVDIDLEKFFDNVPQDR | |
| | LMSLVHVIINDGDTESLIRKYLKAGVMTPQGYEETRLGTPQGGNLSPLLSNIMLNELDKELEARGLRFTRYADDCV | |
| | IAVKSETSAKRVMRTVSDWIQRKLGLKVNMTKTHITRPLELKYLGFGFYKDSKTKEWKCRAHQDSIVKIKRRLKE | |
| | LTCRKTPGKVREKIEKINQVTRGWINYYALGSMKTAMTAIDAHLRTRLRVIIWKRWKVPKKRQWGLQKLGIGKD | |
| | LARLTSYCGDRYQWVVTKTCVVRAISEEVLAKAGLISCLEYYNKRHALKLC | |
| 21535 | MSELLDKILHKDNMNEAYKKVCANKGAGGVDGVSTEELGEYIKTNWPTIKEQILRREYKPQPVRRVEIPKPNGGK | 63 |
| | RKLGIPTAMDRVIQQAIVQVIGPICDPHFSEFSYGFRPNRSCEMAIRKVLEYLNEDYTWIVDIDLEKFFDNVPQDRLL | |
| | SYVHILINDGDTESLIRKYLKVGVMTPEGYEHTEMGTPQGGNLSPLLSNIMLNELDKELENRGLHFARYADDCIIA | |
| | VKSEASAKRVMNSITSWIERKLGLKVNAEKTKIVRPTKVKYLGFGFYKDTKTLEWKCKPHQDSVAKFKRRLKAL | |
| | TQRKNSMRLGIRIKKINQVTRGWINYFALGSMKMAISDIDAHLRTRLRTIIWKQWKVPSKRQWGLQKLGVSKDLA | |
| | RLTSYCGDRYQWVVTKTCVKRAISKEVLGRAGLISCLDYYNERHALKLN | |
| 21536 | MSELLDKILDRENMNEAYKRVCANKGAGGVDGVSTEELGDYIRKNWPEIKEQIKRREYKPQPVRRVEIPKPSGGK | 64 |
| | RKLGIPTAMDRVIQQAIVQAIGPICDPHFSEFSYGFRPKRSCEMAIRKVLEYLNEDYTWIVDIDLEKFFDNVPQDRL | |
| | MSYLHILINDGDTESLICKYLKAGVMTPEGYERTEMGTPQGGNLSPLLSNIMLNELDKELEHRGLHFARYADDCII | |
| | AVKSEASAKRVMYSITSWIERKLGLKVNAEKTKIVRPTNLKYLGFGFYKDYKADEWKCKPHQEAIAKFKRKLKA | |
| | LTQRKSSMKLGLRIEKINQVTRGWINYFALGSMKMALADIDAHLRTRLRTIIWKQWKVPSKRQWGLQKLGVNKD | |
| | LARLTSYRGNRYQWVVTKTCVVRAISKEVLGKAGLISCLDYYTERHALKLS | |
| 21537 | MSELLEKILDKRNMNEAYKKVCANKGAGGVDGMEIDELDGYIRGNWESIKEQIRKRSYTPQPVRRVEIPKSNGSK | 65 |
| | RKLGIPTVMDRVIQQGIAQVISPMCEPLFSGNSYGFRPNRSCEMAIREMLVFLNEGYEWIVDIDLEKFFDNVPQDRL | |
| | MSLVHNIINDGDTESLIRKYLKAGVMIQGRYEKTDRGTPQGGNLSPLLSNIMLNELDKELEKRGLRFVRYADDCVI | |
| | AVRSEASAKRVMYSITDWIERKLGLKVNAEKTHITRPGKLKYLGFGFYKDPKAKEWKSRPHGESVAKFRRTLKKL | |
| | TNRSQSMAFAERVQKLNRVIRGWINYFALGSMKTAMNDIDAHLRTRLRVIIWKQWKVPSKRQWGLQKLGIGEDL | |
| | ARVTAYCGNRYQWIVTKTCVVRAISKEKLSQAGLVSCYDYYMERHALKLC | |
| 21538 | MSELLDRILSRDNMNLAYKRVKANKGAGGIDDVEVDELYSYIKENWKSIEGQIRQRTYKPQPVRRVEIPKPNGGK | 66 |
| | RELGIPTVMDRVIQQAIVQVISPMCEPYFSDRSYGFRPNRSCETAIRKVLEYLNDGYTWIVDIDLEKFFDNVPQDRL | |
| | MSLVHRIIDDGDTESLIRKYLKAGVMVRGKYERTELGTPQGGNLSPLLSNIMLNELDRELEKRGLNFTRYADDCII | |
| | AVRSEASAKRVMHRITEWIERKLGLKVNATKTKITRPSGLKYLGFGFYRDPKADEWKCKPHKDSIAKFKHKLKQL | |
| | TERRWSINFRVRIAKINQVTRGWINYYALGSMKTAMAEIDAHLRTRLRMIIWKQWKVPAKRQWGLQKLGIGKDL | |
| | ARVTAYCGNRYYWVVTKTCVVRAISKEKLALAGIVSLSDYYAERHMLKLI | |
| 21539 | MSEMLERILDDENIKTAYKRVYANKGAGGVDGVTTEELEEYMRVNWRSIKEQIRERKYNPQAVKRVEIPKPNGGI | 67 |
| | RKLGIPTVIDRVIEQAITQILTPIFDPLFSDSSYGFRPNRRCEQAIIKLLEHFNDGYVWIVDIDLEKFFDNVPQDKLMS | |
| | YVGRVIHDGDTESLIRKYLKAGVMVNGRYEATNLGTPQGGNLSPLLSNIMLDELDKELENRGLHFTRYADDCVIV | |
| | LKSKAAATRVMYSITDWIERKLGLKVNATKTKITPPNKLKYLGFGFWKDKQEWKARPHEDSVEKLKRKLKALCK | |
| | RNWSIDLTTRIKKINEVTRGWINYFRIASMKSKLQNIDEHLRTQLRVIIWKQWKVPKKREWGLAKLGVGKDLARL | |
| | TAYCGDRYQWVVTKTCVVRAISKEKLTKKGLVSCLDYYAERRHILNLN | |
| 21540 | MSELLEKILDRDNMNAAYKRVCANKGAGGIDGVTVEELSDYIKENWGSIREQIRQRKYKPQPVRRVEIPKLNGGV | 68 |
| | RKLGIPTVMDRVIQQGIVQVVSPMCEPMFSERSYGFRPNRSCEMAIRQLLVYLNEGYEWIVDIDLEKFFDNVPQDR | |
| | LMSLVHDIVKDGDTESLIRKYLKAGVMTRQGYEETNLGTPQGGNLSPLLSNIMLDKLDKELEARGLRFARYADDC | |
| | VIAVKSEASAKRVMHTVTDWIQRKLGLKVNMTKTHITRPMKLKYLGFGFYKDNKTEEWKCRPHKDSIVRLKSKL | |
| | KELTCRRMPGKVSEKIKRINQVTRGWINYYALGSMKTVMAEVDAHLRTRLRMNIWKQWKVPKKRQWGLQKLG | |
| | VDKDSARLTAYYGNHYYKVVKGTCVKRAISKEVLARAGLI | |
| 21541 | MLEKILSKDNMNKAYKAVAANKGSSGVDNVTIEELGSFIKEYWIEIREQIRQRQYKPQPVRRVEIPKPNGGIRKLGI | 69 |
| | PTVMDRVIQQAIVQVISPICEPYFSDRSYGFRPGRSCEMAIVKLLEYLNDGYEWIVDIDLEKFFDNVPQDKLMSYVH | |
| | NIIDDGDTESLIRKYLKAGVMTPEGYEETRLGTPQGGNLSPLLSNIMLNELDKELEARGLNFARYADDCVIAAKTD | |
| | MSARRIMRNITSWIERKLGLKVNATKTKVVRPTKLKYLGFGFYKDIQTKKWMSRPHEESVEKFKRKLKQLTCRST | |
| | SMKFKIRINKLNQVIRGWINYFSLGSMKTKMKEIDEHLRTRLRVVLWKQWKKPSKRQWALQKLGIGSDLARQTA | |
| | YMGDHYQWVVTKTCVVRAISKENLGQFGLVSCLDYYTERHSLKFN | |
| 21542 | MLQLLEEVLDRNNMNLAYEKVYANKGAGGVDGVTLDELKAYIKENWPDVQTQIRNRTYQPKPVKRVEIPKANG | 70 |
| | GTRNLGIPTVMDRVIQQAMVQVLSPICEPHFSEYSYGFRPKRSCEMAILKLLDYFNDGFLWIVDIDLEKFFDNVPQD | |
| | RLMSYVHNIINDGDTESLIRKYLKAGVMINGVKHKTEVGTPQGGNLSPLLSNIMLTELDKELEARGLKFTRYADD | |
| | CIIVVKSEASAKRVMRTITDWIERKLGLKVNMTKTQVTGPTKLKYLGFGFYFDYKSKTWKTRPHEDSVTKFKRKL | |
| | KQLTKRNWSINLRERIKKINGVIRGWINYFSLCSMKTHMEKIDRHLRTRIRVIVWKQWKVPSKRQWGLQKLGIGR | |
| | DLAKQTSYMGNHYQWVVTKTCVVRAISKEKLAQAGLVHCLDYYLNQHTLKFNRTAVCRTACTVV | |
| 21543 | MSELLEKILNRENMNKAYKRVKANKGTSGIDEITIEDAYVYIKENWESIRAEITERKYKPQPVKRVEIPKPNGGTRN | 71 |
| | LGIPTVMDRIIQQAMVQVLSPICETFFSDYSYGFRPNRSCEQAINKLLEYINDGYEWIVDIDLEKFFDNVPQDKLMS | |
| | YVHIIINDGDTESLIRKYLKAGIMINGKYEKSEKGTPQGGNLSPLLSNIILNELDKELESRGLHFTRYADDCVIAVKS | |
| | RASANRVMHTITKWIEHKLGLKVNATAAAGLKVNATKTHITRPNKLKYLGFGFYYDTKAEKYCARPHASSIQRF | |
| | KRKLKQLTIRKNTMALKERIRRLNQVIRGWINYYSICNMKTHMTRIDEHLRTRLRVIIWKQWKVPSKRQWGLQKL | |
| | GISKDRARQTSYMGDHYQWVVTKTCVVRAISKEKLAQKGLVSCLDYYIERHALKLKRTAVYGTVRTVV | |
| 21544 | MSELLEKILNRDNMNQAYKRVKANKGTSGIDEVTVNEAYGYIEENWERIKQEITERRYKPQPVKRVEIPKPNGGT | 72 |
| | RNLGIPTVMDRIIQQAMVQVLSPMCEEIFSEYSYGFRPGRSCEQAIIKVLEYFNDGYGWIVDIDLEKFFDNVPQDKL | |
| | MSYVHTIINDGDTESLIRKFLKAGIMINGRYEESEKGTPQGGNLSPLLSNIILSELDKELESRGLKFVRYADDCVIAV | |
| | KSRASANRVMHTITSWIEGKLGLKVNATKTQITKPNKLKYLGFGFYYHPKEKKYCARPHESSIQQFKRKLKRLTIR | |
| | KNTMTLEVRIKQLNQVIRGWINYYAIGNMKTHMARIDSHLRTRLRVIIWKQWKVPSKRQWGLQKLGINKDRARQ | |
| | TSYMGDHYQWVVTKTCVVRAISKERLTQKGLVSCLDYYMDRHNLKLERTAVYGTVRTVV | |
| 21551 | MRNVNEQILHYLGRIHENGKEKAHKAMSELLEKILSKDNMNAAYKRVYANKGAGGVDDVTVGELGGYIKENWE | 73 |
| | SIREQIRRREYKPQPVRRVEIPKPNGGVRELGIPTVMDRVIQQGIVQVISPVCEPLFSECSYGFRPNRSCEMAVRQLLI | |
| | YLNEGYEWIVDIDLERFFDNVPQDKLMSLVHNIINDGDTESLIRKYLKAGVMTPDGYEETKLGTPQGGNLSPLLSN | |
| | IMLNELDKELEARGLRFTRYADDCVIAVRSESSAKRVMRTVSDWIQRKLGLKVNTTKTHITRPQKLKYLGFGFYK | |
| | DSKAKEWKCRPHQESVKKLKRRLKELTCRKMPGTVTGRIAKINQVTRGWINYYALGSMKTAMTEIDAHLRTRLR | |
| | IIIWKQWKVPKKRQWGLQKLGVDKDLARLTAYCGDRYYWVATKTCVVRAISKDVFAKAGLISCLDYYNERHAL | |
| | KLC | |
| 21560 | MSELLEKILSKENMNTAYKRVCANKGAGGVDGVTVEELGDYIKEHWRSIREQIRQRQYKPQPVRRVEIPKQNGG | 74 |
| | VRELGIPTVMDRVIQQGIVQVINPMCEPLFSEWSYGFRPNRSCEMAIRQLLIYLNEGYEWIVDIDLEKFFDNVPQDR | |
| | LMSLVHDIINDGDTESLIRKYLKAGVMTPQGYEETRLGTPQGGNLSPLLSNIMLNELDKELEARGLRFTRYADDCV | |
| | IAVKSESSAKRVMRTVSDWIQRKLGLKVNMTKTHITRPLKLKYLGFGFYKDSKTKEWKCRAHQDSIVKLKRKLK | |
| | ELTCRKTPGKVREKIEKINQVTRGWINYYALGSMKTAMTAIDAHLRTRLRVIIWKQWKVPKKRQWGLQKLGVGK | |
| | DLARLTSYCGDRYQWVVTKTCVVRAISKEVLAKAGLISCLEYYNKRHALKLC | |
| 21564 | MSELLEKILSKNNMNAAYKKVCANKGAGGVDNVTVEELGDYIKENWESIREQIRRREYKPQPVRRVEIPKPDGGV | 75 |
| | RKLGIPTVMDRVIQQGIVQVISPMCEPVFSEWSYGFRPNRSCEMAVRQLLVYLNEGYEWIVDIDLERFFDNVPQDR | |
| | LMSLVHDIINDGDTESLIRKYLKAGVMSRQGYEETKLGTPQGGNLSPLLSNIMLNELDKELEARGLRFTRYADDCV | |
| | IVVRSESSAKRVMRTVADWIQRKLGLKVNMTKTHITRPQKLKYLGFGFYRDSRAKEWKCRPHQDSVKKFKRKLK | |
| | ELTCRKMPGTVTGRIVQINQVTRGWINYYALGSMKTAMTEIDAHLRTRLRIIIWKQWKVPKKRQWGLQKLGIGK | |
| | DLARLTAYCGDRYYWVATKTCVVRAISKDVFARSGLISCLDYYNERHALKLC | |
| 21566 | MSELLEKILSKKNMNAAYKKICANKGAGGVDDVTVEELGDYIKENWESIREQIRRREYKPQPVRRVEIPKPDGGV | 76 |
| | RKLGIPTVMDRVIQQGIVQIISPMCEPLFSEWSYGFRPNRSCEMAVRQLLMYLNEGYEWIVDIDLEKFFDNVPQDR | |
| | LMSLVHDIINDGDTESLIRKYLKAGVMTPQGYKETKLGTPQGGNLSPLLSNIMLNELDKELEARGLRFTRYADDC | |
| | VIAVRSESSAKRVMRTVTDWIQRKLGLKVNMTKTHITRPQKLKYLGFGFYKDSKAKEWKCRPHQDSVKKFKRKL | |
| | KELTCRKMPGTVTGKIVKINQVTRGWVNYYALGSMKTAMTEIDAHLRTRLRIIIWKQWKAPKKRQWGLQKLGID | |
| | KDLARLTAYCGDRYYWVATKTCVNRAISKKVLTKAGLVSCLDYYNERHALKLC | |
| 21572 | MSELLEKILSQNNMNAAYKKVCANKGAGGVDNVTVEELGDYIKENWESIREQIRRREYKPQPVRRVEIPKPDGGV | 77 |
| | RKLGIPTVMDRVIQQGIVQVISPMCEPMFSEWSYGFRPNRSCEMAIRQLLVYLNEGYEWIVDIDLERFFDNVPQDR | |
| | LMSLVHDIINDGDTESLIRKYLKAGVMSRQGYEETKLGTPQGGNLSPLLSNIMLNELDKELGARGLRFTRYADDC | |
| | VIAVSSESSAKRVMRTVADWIQRKLGLKVNMTKTHITRPQKLKYLGFGFYKDSREKQWKCRPHQDSVKKFKRKL | |
| | KELTCRKMPGTVTGKIVQINQVTRGWINYYALGSMKTAMTEIDAHLRTRLRIIIWKQWKVPKKRQWGLQKLGIG | |
| | KDLARLTAYCGDRYYWVATKTCVVRAISKDVFAKAGLISCLDYYNEHHALKLC | |
| 21573 | MSRYCNHLGQIHENGKEKAHKAMSELLEKVLSKDNMNAAYKRVCANKGAGGVDDVTVEELGDYIKENWEGIR | 78 |
| | EQIRQREYRPQPVRRVEIPKPNGGVRKLGIPTVMDRVIQQGIVQIISPMCEPLFSERSYGFRPDRSCEMAVRQLLVYL | |
| | NEGYEWIVDIDLEKFFDNVPQDRLMSLVHDIIKDGDTESLIRKYLKAGVMTPQGYEETNLGTPQGGNLSPLLSNIM | |
| | LNELDKELEERGLRFTRYADDCVIAVKSEASAKRVMRTVTGWIQRKLGLKVNMTKTRITRPQRLKYLGFGLYKD | |
| | SKAKEWKCRPHQESVKKFKGRLKELTCRKMPGTVTGRIAEINQVTRGWINYYALGSMKTAMTETDAHLRTRLRII | |
| | IWKQWKVPKKRQWGLQKLGVSKDLARLTAYCGDRYYWVATKTCVVRAISKDVFAKAGLVSCLDYYNERHALK | |
| | LC | |
| 21574 | MSELLEKILSKNNMNAAYKKICANKGAGGVDDVTVEELGDYIKENWESIREQIRRREYKPQPVRRVEIPKSDGGV | 79 |
| | RKLGIPTVMDRVIQQGIVQIISPMCEPLFSEWSYGFRPNRSCEMAVRQLLMYLNEGYEWIVDIDLEKFFDNVPQDR | |
| | LMSLVHDIINDGDTESLIRKYLKAGVMTPQGYKETKFGTPQGGNLSPLLSNIMLNELDKELEARGLRFTRYADDCV | |
| | IAVRSESSAKRVMRTVTDWIQRKLGLKVNMTKTHITRPQKLKYLGFGFYKDSKAKEWKCRPHQDSVKKFKRKLK | |
| | ELTCRKMPGTVTGKIVKINQVTRGWVNYYALGSMKTAMTEIDAHLRTRLRIIIWKQWKAPKKRQWGLQKLGIDK | |
| | DLARLTAYCGDRYYWVATKTCVNRAISKKVLTKAGLVSCLDYYNERHALKLC | |

TABLE 4 provides exemplary engineered RDDP sequences.

TABLE 4
Exemplary Engineered RDDPs

| | | SEQ |
|---|---|---|
| RDDP | | ID |
| Ref | Amino Acid Sequence | NO: |
| 2691319 | MSELLEKILSKRNMNTAYKRVCANKGAGGVDDVTVEELGAYVKENWESIREQIRRREYTPQPVRRVEIPKPRG | 80 |
| (D12R- | GERKLGIPTVMDRVIQQGIVQIISPMCEPLFSKWSYGFRPNRSCEMAIRQLLEYLNEGYEWIVDIDLEKFFDNVP | |
| D72R- | QDRLMSLVHDIIKDGDTESLIRKYLKAGAMTPQGYKETKLGTPQGGRLSPLLSNIMLNELDKELEARGLRFTRY | |
| N195R) | ADDCVIAVKSESSAKRVMRTVTDWIQRKLGLKVNMTKSQITRPQKLKYLGFGFHKDSKAKEWKCRPHQESVK | |
| | KFKVRLKELTCRKKPGTVTSKIAKINQVTRGWINYYALGSMKTAMTEIDAHLRTRLRVIIWKQWKVPKKRQW | |
| | GLQKLGVSKDLARLTAYCGDSYYWVATKTCVVRAISKEVFAKSGLVSCLDYYNERHALKLC | |
| 2691319 | MSELLEKILSKRNMNTAYKRVCARKGAGGVDDVTVEELGAYVKENWESIREQIRRREYTPQPVRRVEIPKPRG | 81 |
| (D12R- | GERKLGIPTVMDRVIQQGIVQIISPMCEPLFSKWSYGFRPKRSCEMAIRQLLEYLNEGYEWIVDIDLEKFFDNVP | |
| N24R- | QDRLMSLVHDIIKDGDTESLIRKYLKAGAMTPQGYKETKLGTPQGGNLSPLLSNIMLNELDKELEARGLRFTRY | |
| D72R- | ADDCVIAVKSESSAKRVMRTVTDWIQRKLGLKVNMTKSQITRPQKLKYLGFGFHKDSKAKEWKCRPHQESVK | |
| N114K) | KFKVRLKELTCRKKPGTVTSKIAKINQVTRGWINYYALGSMKTAMTEIDAHLRTRLRVIIWKQWKVPKKRQW | |
| | GLQKLGVSKDLARLTAYCGDSYYWVATKTCVVRAISKEVFAKSGLVSCLDYYNERHALKLC | |
| 2691319 | MSELLEKILSKRNMNTAYKRVCARKGAGGVDDVTVEELGAYVKENWESIREQIRRREYTPQPVRRVEIPKPRG | 82 |
| (D12R- | GERKLGIPTVMDRVIQQGIVQIISPMCEPLFSKWSYGFRPNRSCEMAIRQLLEYLNEGYEWIVDIDLEKFFDNVP | |
| N24R- | QDRLMSLVHDIIKDGDTESLIRKYLKAGAMTPQGYKETKLGTPQGGRLSPLLSNIMLNELDKELEARGLRFTRY | |
| D72R- | ADDCVIAVKSESSAKRVMRTVTDWIQRKLGLKVNMTKSQITRPQKLKYLGFGFHKDSKAKEWKCRPHQESVK | |
| N195R) | KFKVRLKELTCRKKPGTVTSKIAKINQVTRGWINYYALGSMKTAMTEIDAHLRTRLRVIIWKQWKVPKKRQW | |
| | GLQKLGVSKDLARLTAYCGDSYYWVATKTCVVRAISKEVFAKSGLVSCLDYYNERHALKLC | |
| 2691319 | MSELLEKILSKRNMNTAYKRVCANKGAGGVDDVTVEELGAYVKENWESIREQIRRREYTPQPVRRVEIPKPRG | 83 |
| (D12R- | GERKLGIPTVMDRVIQQGIVQIISPMCEPLFSKWSYGFRPKRSCEMAIRQLLEYLNEGYEWIVDIDLEKFFDNVP | |
| D72R- | QDRLMSLVHDIIKDGDTESLIRKYLKAGAMTPQGYKETKLGTPQGGRLSPLLSNIMLNELDKELEARGLRFTRY | |
| N114K- | ADDCVIAVKSESSAKRVMRTVTDWIQRKLGLKVNMTKSQITRPQKLKYLGFGFHKDSKAKEWKCRPHQESVK | |
| N195R) | KFKVRLKELTCRKKPGTVTSKIAKINQVTRGWINYYALGSMKTAMTEIDAHLRTRLRVIIWKQWKVPKKRQW | |
| | GLQKLGVSKDLARLTAYCGDSYYWVATKTCVVRAISKEVFAKSGLVSCLDYYNERHALKLC | |
| 2691319 | MSELLEKILSKRNMNTAYKRVCARKGAGGVDDVTVEELGAYVKENWESIREQIRRREYTPQPVRRVEIPKPRG | 84 |
| (D12R- | GERKLGIPTVMDRVIQQGIVQIISPMCEPLFSKWSYGFRPKRSCEMAIRQLLEYLNEGYEWIVDIDLEKFFDNVP | |
| N24R- | QDRLMSLVHDIIKDGDTESLIRKYLKAGAMTPQGYKETKLGTPQGGRLSPLLSNIMLNELDKELEARGLRFTRY | |
| D72R- | ADDCVIAVKSESSAKRVMRTVTDWIQRKLGLKVNMTKSQITRPQKLKYLGFGFHKDSKAKEWKCRPHQESVK | |
| N114K- | KFKVRLKELTCRKKPGTVTSKIAKINQVTRGWINYYALGSMKTAMTEIDAHLRTRLRVIIWKQWKVPKKRQW | |
| N195R) | GLQKLGVSKDLARLTAYCGDSYYWVATKTCVVRAISKEVFAKSGLVSCLDYYNERHALKLC | |
| 2691323 | MSELLERILSNRNMNAAYKRVCANKGAGGVDGVTVEELGDYIKENWSSIREQIRRRQYKPQPVRRVEIPKPRG | 85 |
| (D12R- | GVRNLGIPTVMDRVIQQGIVQVLSPMCEPLFSERSYGFRPNRSCEMAIRQLLVYLNEGCEWIVDIDLEKFFDNVP | |
| N72R- | QDRLMSLVHDIINDGDTESLIRKYLKAGVMTPQGYEETKLGTPQGGRLSPLLSNIMLNELDKELEKRGLRFTRY | |
| N195R) | ADDCVIAVKSESSAKRVMRTVADWIQRKLGLKVNMTKTHITRPTKLKYLGFGFYRDNKAKEWKCRPHQDSV | |
| | AKFKRKLKELTCRKTPGTVTGRIAKINQVTRGWINYYAIGSMKTTMIEIDAHLRTRLRVIIWKQWKVPKKRQW | |
| | GLQKLGIGKDLARLTSYYGDRYQWIVTKTCVARAISKEVLAKAGLVSCLDYYNERHALKLC | |
| 2691323 | MSELLERILSNRNMNAAYKRVCARKGAGGVDGVTVEELGDYIKENWSSIREQIRRRQYKPQPVRRVEIPKPRG | 86 |
| (D12R- | GVRNLGIPTVMDRVIQQGIVQVLSPMCEPLFSERSYGFRPKRSCEMAIRQLLVYLNEGCEWIVDIDLEKFFDNVP | |
| N24R- | QDRLMSLVHDIINDGDTESLIRKYLKAGVMTPQGYEETKLGTPQGGNLSPLLSNIMLNELDKELEKRGLRFTRY | |
| N72R- | ADDCVIAVKSESSAKRVMRTVADWIQRKLGLKVNMTKTHITRPTKLKYLGFGFYRDNKAKEWKCRPHQDSV | |
| N114K) | AKFKRKLKELTCRKTPGTVTGRIAKINQVTRGWINYYAIGSMKTTMIEIDAHLRTRLRVIIWKQWKVPKKRQW | |
| | GLQKLGIGKDLARLTSYYGDRYQWIVTKTCVARAISKEVLAKAGLVSCLDYYNERHALKLC | |
| 2691323 | MSELLERILSNRNMNAAYKRVCARKGAGGVDGVTVEELGDYIKENWSSIREQIRRRQYKPQPVRRVEIPKPRG | 87 |
| (D12R- | GVRNLGIPTVMDRVIQQGIVQVLSPMCEPLFSERSYGFRPNRSCEMAIRQLLVYLNEGCEWIVDIDLEKFFDNVP | |
| N24R- | QDRLMSLVHDIINDGDTESLIRKYLKAGVMTPQGYEETKLGTPQGGRLSPLLSNIMLNELDKELEKRGLRFTRY | |
| N72R- | ADDCVIAVKSESSAKRVMRTVADWIQRKLGLKVNMTKTHITRPTKLKYLGFGFYRDNKAKEWKCRPHQDSV | |
| N195R) | AKFKRKLKELTCRKTPGTVTGRIAKINQVTRGWINYYAIGSMKTTMIEIDAHLRTRLRVIIWKQWKVPKKRQW | |
| | GLQKLGIGKDLARLTSYYGDRYQWIVTKTCVARAISKEVLAKAGLVSCLDYYNERHALKLC | |
| 2691323 | MSELLERILSNRNMNAAYKRVCANKGAGGVDGVTVEELGDYIKENWSSIREQIRRRQYKPQPVRRVEIPKPRG | 88 |
| (D12R- | GVRNLGIPTVMDRVIQQGIVQVLSPMCEPLFSERSYGFRPKRSCEMAIRQLLVYLNEGCEWIVDIDLEKFFDNVP | |
| N72R- | QDRLMSLVHDIINDGDTESLIRKYLKAGVMTPQGYEETKLGTPQGGRLSPLLSNIMLNELDKELEKRGLRFTRY | |
| N114K- | ADDCVIAVKSESSAKRVMRTVADWIQRKLGLKVNMTKTHITRPTKLKYLGFGFYRDNKAKEWKCRPHQDSV | |
| N195R) | AKFKRKLKELTCRKTPGTVTGRIAKINQVTRGWINYYAIGSMKTTMIEIDAHLRTRLRVIIWKQWKVPKKRQW | |
| | GLQKLGIGKDLARLTSYYGDRYQWIVTKTCVARAISKEVLAKAGLVSCLDYYNERHALKLC | |
| 2691323 | MSELLERILSNRNMNAAYKRVCARKGAGGVDGVTVEELGDYIKENWSSIREQIRRRQYKPQPVRRVEIPKPRG | 89 |
| (D12R- | GVRNLGIPTVMDRVIQQGIVQVLSPMCEPLFSERSYGFRPKRSCEMAIRQLLVYLNEGCEWIVDIDLEKFFDNVP | |
| N24R- | QDRLMSLVHDIINDGDTESLIRKYLKAGVMTPQGYEETKLGTPQGGRLSPLLSNIMLNELDKELEKRGLRFTRY | |
| N72R- | ADDCVIAVKSESSAKRVMRTVADWIQRKLGLKVNMTKTHITRPTKLKYLGFGFYRDNKAKEWKCRPHQDSV | |
| N114K- | AKFKRKLKELTCRKTPGTVTGRIAKINQVTRGWINYYAIGSMKTTMIEIDAHLRTRLRVIIWKQWKVPKKRQW | |
| N195R) | GLQKLGIGKDLARLTSYYGDRYQWIVTKTCVARAISKEVLAKAGLVSCLDYYNERHALKLC | |

TABLE 5 provides exemplary fusion protein sequences.

TABLE 5
Exemplary Fusion Protein Sequences

| | | SEQ |
|---|---|---|
| Fusion | | ID |
| protein | Amino Acid Sequence | NO: |
| 2691319 | DKKYSIGLDIGTNSVGWAVITDEYKVPSKKFKVLGNTDRHSIKKNLIGALLFDSGETAEATRLKRTARRRYTRRK | 90 |
| -RDDP | NRICYLQEIFSNEMAKVDDSFFHRLEESFLVEEDKKHERHPIFGNIVDEVAYHEKYPTIYHLRKKLVDSTDKADL | |
| fusion at | RLIYLALAHMIKFRGHFLIEGDLNPDNSDVDKLFIQLVQTYNQLFEENPINASGVDAKAILSARLSKSRRLENLIA | |
| N term | QLPGEKKNGLFGNLIALSLGLTPNFKSNFDLAEDAKLQLSKDTYDDDLDNLLAQIGDQYADLFLAAKNLSDAIL | |
| | LSDILRVNTEITKAPLSASMIKRYDEHHQDLTLLKALVRQQLPEKYKEIFFDQSKNGYAGYIDGGASQEEFYKFIK | |
| | PILEKMDGTEELLVKLNREDLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEISGVEDRFNASLGTYHDLLKIIKD | |
| | KDFLDNEENEDILEDIVLTLTLFEDREMIEERLKTYAHLFDDKVMKQLKRRRYTGWGRLSRKLINGIRDKQSGK | |
| | TILDFLKSDGFANRNFMQLIHDDSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQLQNEKLYLYYLQNGRDMYVDQ | |
| | ELDINRLSDYDVDAIVPQSFLKDDSIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQEIGKATAKYFFYSNIMNFFKTE | |
| | ITLANGEIRKRPLIETNGETGEIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPEDNEQKQLFVEQHKHYLDEIIEQISE | |
| | FSKRVILADANLDKVLSAYNKHRDKPIREQAENIIHLFTLTNLGAPAAFKYFDTTIDRKRYTSTKEVLDATLIHQS | |
| | ITGLYETRIDLSQLGGD | |
| 2691323 | MSELLERILSNDNMNAAYKRVCANKGAGGVDGVTVEELGDYIKENWSSIREQIRRRQYKPQPVRRVEIPKPNGG | 91 |
| -RDDP | VRNLGIPTVMDRVIQQGIVQVLSPMCEPLFSERSYGFRPNRSCEMAIRQLLVYLNEGCEWIVDIDLEKFFDNVPQ | |
| fusion at | DRLMSLVHDIINDGDTESLIRKYLKAGVMTPQGYEETKLGTPQGGNLSPLLSNIMLNELDKELEKRGLRFTRYA | |
| N term | DDCVIAVKSESSAKRVMRTVADWIQRKLGLKVNMTKTHITRPTKLKYLGFGFYRDNKAKEWKCRPHQDSVAK | |
| | FKRKLKELTCRKTPGTVTGRIAKINQVTRGWINYYAIGSMKTTMIEIDAHLRTRLRVIIWKQWKVPKKRQWGLQ | |
| | KLGIGKDLARLTSYYGDRYQWIVTKTCVARAISKEVLAKAGLVSCLDYYNERHALKLCGSSGGSPAGSPTSTEE | |
| | GTSESATPESGPGTSTEPSEGSAPGSPAGSGGGSDKKYSIGLDIGTNSVGWAVITDEYKVPSKKFKVLGNTDRHSI | |
| | KKNLIGALLFDSGETAEATRLKRTARRRYTRRKNRICYLQEIFSNEMAKVDDSFFHRLEESFLVEEDKKHERHPIF | |
| | GNIVDEVAYHEKYPTIYHLRKKLVDSTDKADLRLIYLALAHMIKFRGHFLIEGDLNPDNSDVDKLFIQLVQTYNQ | |
| | LFEENPINASGVDAKAILSARLSKSRRLENLIAQLPGEKKNGLFGNLIALSLGLTPNFKSNFDLAEDAKLQLSKDT | |
| | YDDDLDNLLAQIGDQYADLFLAAKNLSDAILLSDILRVNTEITKAPLSASMIKRYDEHHQDLTLLKALVRQQLPE | |
| | KYKEIFFDQSKNGYAGYIDGGASQEEFYKFIKPILEKMDGTEELLVKLNREDLLRKQRTFDNGSIPHQIHLGELHA | |
| | ILRRQEDFYPFLKDNREKIEKILTFRIPYYVGPLARGNSRFAWMTRKSEETITPWNFEEVVDKGASAQSFIERMTN | |
| | FDKNLPNEKVLPKHSLLYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFEDREMIEERLKTYAHLFDDKV | |
| | MKQLKRRRYTGWGRLSRKLINGIRDKQSGKTILDFLKSDGFANRNFMQLIHDDSLTFKEDIQKAQVSGQGDSLH | |
| | EHIANLAGSPAIKKGILQTVKVVDELVKVMGRHKPENIVIEMARENQTTQKGQKNSRERMKRIEEGIKELGSQIL | |
| | KEHPVENTQLQNEKLYLYYLQNGRDMYVDQELDINRLSDYDVDAIVPQSFLKDDSIDNKVLTRSDKNRGKSDN | |
| | VPSEEVVKKMKNYWRQLLNAKLITQRKFDNLTKAERGGLSELDKAGFIKRQLVETRQITKHVAQILDSRMNTK | |
| | YDENDKLIREVKVITLKSKLVSDFRKDFQFYKVREINNYHHAHDAYLNAVVGTALIKKYPKLESEFVYGDYKV | |
| | YDVRKMIAKSEQEIGKATAKYFFYSNIMNFFKTEITLANGEIRKRPLIETNGETGEIVWDKGRDFATVRKVLSMP | |
| | QVNIVKKTEVQTGGFSKESILPKRNSDKLIARKKDWDPKKYGGFDSPTVAYSVLVVAKVEKGKSKKLKSVKEL | |
| | LGITIMERSSFEKNPIDFLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGD | |
| 2691335 | MLDRDNLRLAYKRVVRNGGAPGVDGVTVAALQSYLNTHWDRVKNELLAGTYRPAPVRRVEIPKPGGGVRLL | 92 |
| -RDDP | GIPTVMDRFLQQALLQVMNPIFDAHFSWYSYGFRPGKRAHDAVMQAQRYIRDGYRWVVDLDLEKFFDRVNHD | |
| fusion at | MLMARVARKVKDKRVLKLIRAYLNAGVMINGVCQRTEEGTPQGGPLSPLLANILLDDLDKELTRRGLRFVRYA | |
| N term | DDCNIFVASKRAGERVMESAIRFLEGKLKLKVNRDKSAVDRPWKRKFLGFSFLSDKEATIRLAPKTISRFKERVR | |
| | EITSRSRPIPMEERIRRLNQYTMGWVSYFRLASMKNHCERFDQWIRRRLRMCLWKQWKRVRTRIRELRALGVP | |
| | DWACFAMANSRRGPWEMSRNINNALPTSYWEAKGLKSMLTRYMVLRQPFGTAWCGPACQVVGSSGGSPAGS | |
| | PTSTEEGTSESATPESGPGTSTEPSEGSAPGSPAGSGGGSDKKYSIGLDIGTNSVGWAVITDEYKVPSKKFKVLGN | |
| | TDRHSIKKNLIGALLFDSGETAEATRLKRTARRRYTRRKNRICYLQEIFSNEMAKVDDSFFHRLEESFLVEEDKK | |
| | HERHPIFGNIVDEVAYHEKYPTIYHLRKKLVDSTDKADLRLIYLALAHMIKFRGHFLIEGDLNPDNSDVDKLFIQL | |
| | VQTYNQLFEENPINASGVDAKAILSARLSKSRRLENLIAQLPGEKKNGLFGNLIALSLGLTPNFKSNFDLAEDAKL | |
| | QLSKDTYDDDLDNLLAQIGDQYADLFLAAKNLSDAILLSDILRVNTEITKAPLSASMIKRYDEHHQDLTLLKALV | |
| | RQQLPEKYKEIFFDQSKNGYAGYIDGGASQEEFYKFIKPILEKMDGTEELLVKLNREDLLRKQRTFDNGSIPHQIH | |
| | LGELHAILRRQEDFYPFLKDNREKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPAFLSGEQKKAIVDLLFKTNRKVTVKQL | |
| | KEDYFKKIECFDSVEISGVEDRFNASLGTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFANRNFMQLIHDDSLTFKEDIQKAQVSG | |
| | QGDSLHEHIANLAGSPAIKKGILQTVKVVDELVKVMGRHKPENIVIEMARENQTTQKGQKNSRERMKRIEEGIK | |
| | ELGSQILKEHPVENTQLQNEKLYLYYLQNGRDMYVDQELDINRLSDYDVDAIVPQSFLKDDSIDNKVLTRSDKN | |
| | RGKSDNVPSEEVVKKMKNYWRQLLNAKLITQRKFDNLTKAERGGLSELDKAGFIKRQLVETRQITKHVAQILDS | |
| | RMNTKYDENDKLIREVKVITLKSKLVSDFRKDFQFYKVREINNYHHAHDAYLNAVVGTALIKKYPKLESEFVY | |
| | GDYKVYDVRKMIAKSEQEIGKATAKYFFYSNIMNFFKTEITLANGEIRKRPLIETNGETGEIVWDKGRDFATVRK | |
| | VLSMPQVNIVKKTEVQTGGFSKESILPKRNSDKLIARKKDWDPKKYGGFDSPTVAYSVLVVAKVEKGKSKKLK | |
| | SVKELLGITIMERSSFEKNPIDFLEAKGYKEVKKDLIIKLPKYSLFELENGRKRMLASAGELQKGNELALPSKYVN | |
| | FLYLASHYEKLKGSPEDNEQKQLFVEQHKHYLDEIIEQISEFSKRVILADANLDKVLSAYNKHRDKPIREQAENII | |
| | HLFTLTNLGAPAAFKYFDTTIDRKRYTSTKEVLDATLIHQSITGLYETRIDLSQLGGD | |
| 2691339 | ENPIKIDFFEPLEIEPLNLIDVNFVEKMDMDIDLSYEQDSLLKQNLKNLRLDHCNKEEKDAIRKLCFDYRDIFYCE | 93 |
| -RDDP | QIPLSFTSEITHKIKLNDESPIYTKSYRFPEIHKKEVKDQIAKMLDQGIIQHSISPWSSPIWVVPKKLDASGRRKWRL | |
| fusion at | VIDYRKLNEKSCNDRYPLPNITDILDKLGRANYFTTLDLASGFHQIQVDPDDIPKTAFSTEGGHYEFKRMPFGLK | |
| N term | NAPSTFQRVMDNILRGLHNEICLVYLDDIIIFSTSLDEHIQRLKSVFDRLRKSNFKLQLDKSEFLQKTVQYLGHIITP | |
| | QGVKPNPDKVSTIKRFPIPRTQKDIKSFLGLLGYYRRFIKDFAKITKPLTKCLKKNAKVTHDQNFIDAFNTCKEIL | |
| | VNDPILQYPDFSKPFILTTDASDVALGAILSQGTLPNDRPVAYASRTLNETESKYSTIEKELLAIVWACKHFRPYL | |
| | YGRKFTIYTDHRPLTWLFSLKEPNSKLVRWRLKLEEFEYDIIYKKGKLNTNADCLSRVTLNAIDNESMLNNPGDI | |
| | DQDILDTLQNVTQNLQTLQDFQKPSTSNTNDKINIISDIQIKPPDNSQNDNTNTSTQHSTANETTNDGIKIFDEIINN | |
| | KTNQILVFPNVIYKLDIKRETYENHKIVTVKIPIINNEQMILQFLKENTDPKHVYCMYFQSDELYNDFCTVYLKHF | |
| | SEKGPKLIRCLKLVNTVADKEEQILLIKNHHGSKTNHRGINETLEKLKFNYYWKNMKSTVSNFINACDICQRAGS | |
| | SGGSPAGSPTSTEEGTSESATPESGPGTSTEPSEGSAPGSPAGSGGGSDKKYSIGLDIGTNSVGWAVITDEYKVPSK | |
| | KFKVLGNTDRHSIKKNLIGALLFDSGETAEATRLKRTARRRYTRRKNRICYLQEIFSNEMAKVDDSFFHRLEESF | |
| | LVEEDKKHERHPIFGNIVDEVAYHEKYPTIYHLRKKLVDSTDKADLRLIYLALAHMIKFRGHFLIEGDLNPDNSD | |
| | VDKLFIQLVQTYNQLFEENPINASGVDAKAILSARLSKSRRLENLIAQLPGEKKNGLFGNLIALSLGLTPNFKSNF | |
| | DLAEDAKLQLSKDTYDDDLDNLLAQIGDQYADLFLAAKNLSDAILLSDILRVNTEITKAPLSASMIKRYDEHHQ | |
| | DLTLLKALVRQQLPEKYKEIFFDQSKNGYAGYIDGGASQEEFYKFIKPILEKMDGTEELLVKLNREDLLRKQRTF | |
| | DNGSIPHQIHLGELHAILRRQEDFYPFLKDNREKIEKILTFRIPYYVGPLARGNSRFAWMTRKSEETITPWNFEEV | |
| | VDKGASAQSFIERMTNFDKNLPNEKVLPKHSLLYEYFTVYNELTKVKYVTEGMRKPAFLSGEQKKAIVDLLFKT | |
| | NRKVTVKQLKEDYFKKIECFDSVEISGVEDRFNASLGTYHDLLKIIKDKDFLDNEENEDILEDIVLTLTLFEDREM | |
| | IEERLKTYAHLFDDKVMKQLKRRRYTGWGRLSRKLINGIRDKQSGKTILDFLKSDGFANRNFMQLIHDDSLTFK | |
| | EDIQKAQVSGQGDSLHEHIANLAGSPAIKKGILQTVKVVDELVKVMGRHKPENIVIEMARENQTTQKGQKNSRE | |
| | RMKRIEEGIKELGSQILKEHPVENTQLQNEKLYLYYLQNGRDMYVDQELDINRLSDYDVDAIVPQSFLKDDSIDN | |
| | KVLTRSDKNRGKSDNVPSEEVVKKMKNYWRQLLNAKLITQRKFDNLTKAERGGLSELDKAGFIKRQLVETRQI | |
| | TKHVAQILDSRMNTKYDENDKLIREVKVITLKSKLVSDFRKDFQFYKVREINNYHHAHDAYLNAVVGTALIKK | |
| | YPKLESEFVYGDYKVYDVRKMIAKSEQEIGKATAKYFFYSNIMNFFKTEITLANGEIRKRPLIETNGETGEIVWD | |
| | KGRDFATVRKVLSMPQVNIVKKTEVQTGGFSKESILPKRNSDKLIARKKDWDPKKYGGFDSPTVAYSVLVVAK | |
| | VEKGKSKKLKSVKELLGITIMERSSFEKNPIDFLEAKGYKEVKKDLIIKLPKYSLFELENGRKRMLASAGELQKG | |
| | NELALPSKYVNFLYLASHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYETRIDLSQLGGD | |
| 2691355 | VLIRDTSITTNPLPARYHDFSDVFEKKNADRLPEHRLYDCPIDLQEGAHPPFGPIYGLAEPELEALREYLKENLAK | 94 |
| -RDDP | GFIQPSKSPAGAPILFVKKKDGSLRLCVDYRGLNRVTVRNRYPLPLIPELLDRLRTGRVFSKIDLRGAYNLVRIKP | |
| fusion at | GDEWKTAFRTRYGHFEYKVMPFGLTNAPAVFQHMMNHIFREYLDHFVVIYLDDILVFSPSMEEHTRHVRLILTK | |
| N term | LREHGLYAKSEKCEFDRTSVEFLGYVISPAGITMDPRKVATIHNWPIPTRLKEVQSFLGFANFYRRFIDRFSTIVQP | |
| | LISLTRKDVPFVWTATTQHAFDALKQAFMSAPVLVHPNPAKPFQVETDASDFALGAILSQLDDDGTLHPVAYYS | |
| | RKFLASEINYPVYDKELAAIISAFAEWRPYLVGAQHRIQVVTDHKNLLYFASSRTLNRRQARWSIFLADYDFEIIF | |
| | RPGIQHGKADALSRRPDLALCPGDDAYTQQSCSLLKPDQLQLSATFMLHDDSLLQEIAQATKTDTFATEILKRLQ | |
| | DSSPATKRADLHHFTTHDGLLYRNHLLYVPEGPCRTQVLQTCHDDPLAGHFGVAKTLELVSRGFWWPQPWKL | |
| | VKEFVKTCDVCARSKAVHHRPYGLLHPLPIPNRPWASISMDFITDLPPVNGVDTVGSSGGSPAGSPTSTEEGTSES | |
| | ATPESGPGTSTEPSEGSAPGSPAGSGGGSDKKYSIGLDIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESFLVEEDKKHERHPIFGNIV | |
| | DEVAYHEKYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQLSKDTYDD | |
| | DLDNLLAQIGDQYADLFLAAKNLSDAILLSDILRVNTEITKAPLSASMIKRYDEHHQDLTLLKALVRQQLPEKYK | |
| | EIFFDQSKNGYAGYIDGGASQEEFYKFIKPILEKMDGTEELLVKLNREDLLRKQRTFDNGSIPHQIHLGELHAILR | |
| | RQEDFYPFLKDNREKIEKILTFRIPYYVGPLARGNSRFAWMTRKSEETITPWNFEEVVDKGASAQSFIERMTNFD | |
| | KNLPNEKVLPKHSLLYEYFTVYNELTKVKYVTEGMRKPAFLSGEQKKAIVDLLFKTNRKVTVKQLKEDYFKKIE | |
| | CFDSVEISGVEDRFNASLGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDAIVPQSFLKDDSIDNKVLTRSDKNRGKSDNVP | |
| | SEEVVKKMKNYWRQLLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPKLESEFVYGDYKVYD | |
| | VRKMIAKSEQEIGKATAKYFFYSNIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVLVVAKVEKGKSKKLKSVKELLGI | |
| | TIMERSSFEKNPIDFLEAKGYKEVKKDLIIKLPKYSLFELENGRKRMLASAGELQKGNELALPSKYVNFLYLASH | |
| | YEKLKGSPEDNEQKQLFVEQHKHYLDEIIEQISEFSKRVILADANLDKVLSAYNKHRDKPIREQAENIIHLFTLTN | |
| | LGAPAAFKYFDTTIDRKRYTSTKEVLDATLIHQSITGLYETRIDLSQLGGD | |
| 2691319 | DKKYSIGLDIGTNSVGWAVITDEYKVPSKKFKVLGNTDRHSIKKNLIGALLFDSGETAEATRLKRTARRRYTRRK | 95 |
| -RDDP | NRICYLQEIFSNEMAKVDDSFFHRLEESFLVEEDKKHERHPIFGNIVDEVAYHEKYPTIYHLRKKLVDSTDKADL | |
| fusion at | RLIYLALAHMIKFRGHFLIEGDLNPDNSDVDKLFIQLVQTYNQLFEENPINASGVDAKAILSARLSKSRRLENLIA | |
| C term | QLPGEKKNGLFGNLIALSLGLTPNFKSNFDLAEDAKLQLSKDTYDDDLDNLLAQIGDQYADLFLAAKNLSDAIL | |
| | LSDILRVNTEITKAPLSASMIKRYDEHHQDLTLLKALVRQQLPEKYKEIFFDQSKNGYAGYIDGGASQEEFYKFIK | |
| | PILEKMDGTEELLVKLNREDLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEISGVEDRFNASLGTYHDLLKIIKD | |
| | KDFLDNEENEDILEDIVLTLTLFEDREMIEERLKTYAHLFDDKVMKQLKRRRYTGWGRLSRKLINGIRDKQSGK | |
| | TILDFLKSDGFANRNFMQLIHDDSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQLQNEKLYLYYLQNGRDMYVDQ | |
| | ELDINRLSDYDVDAIVPQSFLKDDSIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQEIGKATAKYFFYSNIMNFFKTE | |
| | ITLANGEIRKRPLIETNGETGEIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPEDNEQKQLFVEQHKHYLDEIIEQISE | |
| | FSKRVILADANLDKVLSAYNKHRDKPIREQAENIIHLFTLTNLGAPAAFKYFDTTIDRKRYTSTKEVLDATLIHQS | |
| | ITGLYETRIDLSQLGGDGSSGGSPAGSPTSTEEGTSESATPESGPGTSTEPSEGSAPGSPAGSGGGSMSELLEKILSK | |
| | DNMNTAYKRVCANKGAGGVDDVTVEELGAYVKENWESIREQIRRREYTPQPVRRVEIPKPDGGERKLGIPTVM | |
| | DRVIQQGIVQIISPMCEPLFSKWSYGFRPNRSCEMAIRQLLEYLNEGYEWIVDIDLEKFFDNVPQDRLMSLVHDII | |
| | KDGDTESLIRKYLKAGAMTPQGYKETKLGTPQGGNLSPLLSNIMLNELDKELEARGLRFTRYADDCVIAVKSES | |
| | SAKRVMRTVTDWIQRKLGLKVNMTKSQITRPQKLKYLGFGFHKDSKAKEWKCRPHQESVKKFKVRLKELTCR | |
| | KKPGTVTSKIAKINQVTRGWINYYALGSMKTAMTEIDAHLRTRLRVIIWKQWKVPKKRQWGLQKLGVSKDLA | |
| | RLTAYCGDSYYWVATKTCVVRAISKEVFAKSGLVSCLDYYNERHALKLC | |
| 2691323 | DKKYSIGLDIGTNSVGWAVITDEYKVPSKKFKVLGNTDRHSIKKNLIGALLFDSGETAEATRLKRTARRRYTRRK | 96 |
| -RDDP | NRICYLQEIFSNEMAKVDDSFFHRLEESFLVEEDKKHERHPIFGNIVDEVAYHEKYPTIYHLRKKLVDSTDKADL | |
| fusion at | RLIYLALAHMIKFRGHFLIEGDLNPDNSDVDKLFIQLVQTYNQLFEENPINASGVDAKAILSARLSKSRRLENLIA | |
| C term | QLPGEKKNGLFGNLIALSLGLTPNFKSNFDLAEDAKLQLSKDTYDDDLDNLLAQIGDQYADLFLAAKNLSDAIL | |
| | LSDILRVNTEITKAPLSASMIKRYDEHHQDLTLLKALVRQQLPEKYKEIFFDQSKNGYAGYIDGGASQEEFYKFIK | |
| | PILEKMDGTEELLVKLNREDLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEISGVEDRFNASLGTYHDLLKIIKD | |
| | KDFLDNEENEDILEDIVLTLTLFEDREMIEERLKTYAHLFDDKVMKQLKRRRYTGWGRLSRKLINGIRDKQSGK | |
| | TILDFLKSDGFANRNFMQLIHDDSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQLQNEKLYLYYLQNGRDMYVDQ | |
| | ELDINRLSDYDVDAIVPQSFLKDDSIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQEIGKATAKYFFYSNIMNFFKTE | |
| | ITLANGEIRKRPLIETNGETGEIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPEDNEQKQLFVEQHKHYLDEIIEQISE | |
| | FSKRVILADANLDKVLSAYNKHRDKPIREQAENIIHLFTLTNLGAPAAFKYFDTTIDRKRYTSTKEVLDATLIHQS | |
| | ITGLYETRIDLSQLGGDGSSGGSPAGSPTSTEEGTSESATPESGPGTSTEPSEGSAPGSPAGSGGGSMSELLERILSN | |
| | DNMNAAYKRVCANKGAGGVDGVTVEELGDYIKENWSSIREQIRRRQYKPQPVRRVEIPKPNGGVRNLGIPTVM | |
| | DRVIQQGIVQVLSPMCEPLFSERSYGFRPNRSCEMAIRQLLVYLNEGCEWIVDIDLEKFFDNVPQDRLMSLVHDII | |
| | NDGDTESLIRKYLKAGVMTPQGYEETKLGTPQGGNLSPLLSNIMLNELDKELEKRGLRFTRYADDCVIAVKSES | |
| | SAKRVMRTVADWIQRKLGLKVNMTKTHITRPTKLKYLGFGFYRDNKAKEWKCRPHQDSVAKFKRKLKELTCR | |
| | KTPGTVTGRIAKINQVTRGWINYYAIGSMKTTMIEIDAHLRTRLRVIIWKQWKVPKKRQWGLQKLGIGKDLARL | |
| | TSYYGDRYQWIVTKTCVARAISKEVLAKAGLVSCLDYYNERHALKLC | |
| 2691335 | DKKYSIGLDIGTNSVGWAVITDEYKVPSKKFKVLGNTDRHSIKKNLIGALLFDSGETAEATRLKRTARRRYTRRK | 97 |
| -RDDP | NRICYLQEIFSNEMAKVDDSFFHRLEESFLVEEDKKHERHPIFGNIVDEVAYHEKYPTIYHLRKKLVDSTDKADL | |
| fusion at | RLIYLALAHMIKFRGHFLIEGDLNPDNSDVDKLFIQLVQTYNQLFEENPINASGVDAKAILSARLSKSRRLENLIA | |
| C term | QLPGEKKNGLFGNLIALSLGLTPNFKSNFDLAEDAKLQLSKDTYDDDLDNLLAQIGDQYADLFLAAKNLSDAIL | |
| | LSDILRVNTEITKAPLSASMIKRYDEHHQDLTLLKALVRQQLPEKYKEIFFDQSKNGYAGYIDGGASQEEFYKFIK | |
| | PILEKMDGTEELLVKLNREDLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEISGVEDRFNASLGTYHDLLKIIKD | |
| | KDFLDNEENEDILEDIVLTLTLFEDREMIEERLKTYAHLFDDKVMKQLKRRRYTGWGRLSRKLINGIRDKQSGK | |
| | TILDFLKSDGFANRNFMQLIHDDSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQLQNEKLYLYYLQNGRDMYVDQ | |
| | ELDINRLSDYDVDAIVPQSFLKDDSIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQEIGKATAKYFFYSNIMNFFKTE | |
| | ITLANGEIRKRPLIETNGETGEIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPEDNEQKQLFVEQHKHYLDEIIEQISE | |
| | FSKRVILADANLDKVLSAYNKHRDKPIREQAENIIHLFTLTNLGAPAAFKYFDTTIDRKRYTSTKEVLDATLIHQS | |
| | ITGLYETRIDLSQLGGDGSSGGSPAGSPTSTEEGTSESATPESGPGTSTEPSEGSAPGSPAGSGGGSMLDRDNLRLA | |
| | YKRVVRNGGAPGVDGVTVAALQSYLNTHWDRVKNELLAGTYRPAPVRRVEIPKPGGGVRLLGIPTVMDRFLQ | |
| | QALLQVMNPIFDAHFSWYSYGFRPGKRAHDAVMQAQRYIRDGYRWVVDLDLEKFFDRVNHDMLMARVARK | |
| | VKDKRVLKLIRAYLNAGVMINGVCQRTEEGTPQGGPLSPLLANILLDDLDKELTRRGLRFVRYADDCNIFVASK | |
| | RAGERVMESAIRFLEGKLKLKVNRDKSAVDRPWKRKFLGFSFLSDKEATIRLAPKTISRFKERVREITSRSRPIPM | |
| | EERIRRLNQYTMGWVSYFRLASMKNHCERFDQWIRRRLRMCLWKQWKRVRTRIRELRALGVPDWACFAMAN | |
| | SRRGPWEMSRNINNALPTSYWEAKGLKSMLTRYMVLRQPFGTAWCGPACQVV | |
| 2691339 | DKKYSIGLDIGTNSVGWAVITDEYKVPSKKFKVLGNTDRHSIKKNLIGALLFDSGETAEATRLKRTARRRYTRRK | 98 |
| -RDDP | NRICYLQEIFSNEMAKVDDSFFHRLEESFLVEEDKKHERHPIFGNIVDEVAYHEKYPTIYHLRKKLVDSTDKADL | |
| fusion at | RLIYLALAHMIKFRGHFLIEGDLNPDNSDVDKLFIQLVQTYNQLFEENPINASGVDAKAILSARLSKSRRLENLIA | |
| C term | QLPGEKKNGLFGNLIALSLGLTPNFKSNFDLAEDAKLQLSKDTYDDDLDNLLAQIGDQYADLFLAAKNLSDAIL | |
| | LSDILRVNTEITKAPLSASMIKRYDEHHQDLTLLKALVRQQLPEKYKEIFFDQSKNGYAGYIDGGASQEEFYKFIK | |
| | PILEKMDGTEELLVKLNREDLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEISGVEDRFNASLGTYHDLLKIIKD | |
| | KDFLDNEENEDILEDIVLTLTLFEDREMIEERLKTYAHLFDDKVMKQLKRRRYTGWGRLSRKLINGIRDKQSGK | |
| | TILDFLKSDGFANRNFMQLIHDDSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQLQNEKLYLYYLQNGRDMYVDQ | |
| | ELDINRLSDYDVDAIVPQSFLKDDSIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQEIGKATAKYFFYSNIMNFFKTE | |
| | ITLANGEIRKRPLIETNGETGEIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPEDNEQKQLFVEQHKHYLDEIIEQISE | |
| | FSKRVILADANLDKVLSAYNKHRDKPIREQAENIIHLFTLTNLGAPAAFKYFDTTIDRKRYTSTKEVLDATLIHQS | |
| | ITGLYETRIDLSQLGGDGSSGGSPAGSPTSTEEGTSESATPESGPGTSTEPSEGSAPGSPAGSGGGSENPIKIDFFEPL | |
| | EIEPLNLIDVNFVEKMDMDIDLSYEQDSLLKQNLKNLRLDHCNKEEKDAIRKLCFDYRDIFYCEQIPLSFTSEITH | |
| | KIKLNDESPIYTKSYRFPEIHKKEVKDQIAKMLDQGIIQHSISPWSSPIWVVPKKLDASGRRKWRLVIDYRKLNEK | |
| | SCNDRYPLPNITDILDKLGRANYFTTLDLASGFHQIQVDPDDIPKTAFSTEGGHYEFKRMPFGLKNAPSTFQRVM | |
| | DNILRGLHNEICLVYLDDIIIFSTSLDEHIQRLKSVFDRLRKSNFKLQLDKSEFLQKTVQYLGHIITPQGVKPNPDK | |
| | VSTIKRFPIPRTQKDIKSFLGLLGYYRRFIKDFAKITKPLTKCLKKNAKVTHDQNFIDAFNTCKEILVNDPILQYPD | |
| | FSKPFILTTDASDVALGAILSQGTLPNDRPVAYASRTLNETESKYSTIEKELLAIVWACKHFRPYLYGRKFTIYTD | |
| | HRPLTWLFSLKEPNSKLVRWRLKLEEFEYDIIYKKGKLNTNADCLSRVTLNAIDNESMLNNPGDIDQDILDTLQN | |
| | VTQNLQTLQDFQKPSTSNTNDKINIISDIQIKPPDNSQNDNTNTSTQHSTANETTNDGIKIFDEIINNKTNQILVFPN | |
| | VIYKLDIKRETYENHKIVTVKIPIINNEQMILQFLKENTDPKHVYCMYFQSDELYNDFCTVYLKHFSEKGPKLIRC | |
| | LKLVNTVADKEEQILLIKNHHGSKTNHRGINETLEKLKFNYYWKNMKSTVSNFINACDICQRA | |
| 2691355 | DKKYSIGLDIGTNSVGWAVITDEYKVPSKKFKVLGNTDRHSIKKNLIGALLFDSGETAEATRLKRTARRRYTRRK | 99 |
| -RDDP | NRICYLQEIFSNEMAKVDDSFFHRLEESFLVEEDKKHERHPIFGNIVDEVAYHEKYPTIYHLRKKLVDSTDKADL | |
| fusion at | RLIYLALAHMIKFRGHFLIEGDLNPDNSDVDKLFIQLVQTYNQLFEENPINASGVDAKAILSARLSKSRRLENLIA | |
| C term | QLPGEKKNGLFGNLIALSLGLTPNFKSNFDLAEDAKLQLSKDTYDDDLDNLLAQIGDQYADLFLAAKNLSDAIL | |
| | LSDILRVNTEITKAPLSASMIKRYDEHHQDLTLLKALVRQQLPEKYKEIFFDQSKNGYAGYIDGGASQEEFYKFIK | |
| | PILEKMDGTEELLVKLNREDLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEISGVEDRFNASLGTYHDLLKIIKD | |
| | KDFLDNEENEDILEDIVLTLTLFEDREMIEERLKTYAHLFDDKVMKQLKRRRYTGWGRLSRKLINGIRDKQSGK | |
| | TILDFLKSDGFANRNFMQLIHDDSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQLQNEKLYLYYLQNGRDMYVDQ | |
| | ELDINRLSDYDVDAIVPQSFLKDDSIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQEIGKATAKYFFYSNIMNFFKTE | |
| | ITLANGEIRKRPLIETNGETGEIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPEDNEQKQLFVEQHKHYLDEIIEQISE | |
| | FSKRVILADANLDKVLSAYNKHRDKPIREQAENIIHLFTLTNLGAPAAFKYFDTTIDRKRYTSTKEVLDATLIHQS | |
| | ITGLYETRIDLSQLGGDGSSGGSPAGSPTSTEEGTSESATPESGPGTSTEPSEGSAPGSPAGSGGGSVLIRDTSITTN | |
| | PLPARYHDFSDVFEKKNADRLPEHRLYDCPIDLQEGAHPPFGPIYGLAEPELEALREYLKENLAKGFIQPSKSPAG | |
| | APILFVKKKDGSLRLCVDYRGLNRVTVRNRYPLPLIPELLDRLRTGRVFSKIDLRGAYNLVRIKPGDEWKTAFRT | |
| | RYGHFEYKVMPFGLTNAPAVFQHMMNHIFREYLDHFVVIYLDDILVFSPSMEEHTRHVRLILTKLREHGLYAKS | |
| | EKCEFDRTSVEFLGYVISPAGITMDPRKVATIHNWPIPTRLKEVQSFLGFANFYRRFIDRFSTIVQPLISLTRKDVPF | |
| | VWTATTQHAFDALKQAFMSAPVLVHPNPAKPFQVETDASDFALGAILSQLDDDGTLHPVAYYSRKFLASEINYP | |
| | VYDKELAAIISAFAEWRPYLVGAQHRIQVVTDHKNLLYFASSRTLNRRQARWSIFLADYDFEIIFRPGIQHGKAD | |
| | ALSRRPDLALCPGDDAYTQQSCSLLKPDQLQLSATFMLHDDSLLQEIAQATKTDTFATEILKRLQDSSPATKRAD | |
| | LHHFTTHDGLLYRNHLLYVPEGPCRTQVLQTCHDDPLAGHFGVAKTLELVSRGFWWPQPWKLVKEFVKTCDV | |
| | CARSKAVHHRPYGLLHPLPIPNRPWASISMDFITDLPPVNGVDTV | |

TABLE 6 provides exemplary gRNA sequences designed to be used in combination with CasM.265466 or CasM.265466 variants to modify DMD. With regards to the PAM sequences: N is any nucleotide, V is A, C, or G; B is C, G, or T; and S is G or C. In general, spacer sequences were designed with consideration to a PAM of 5′-NNTR-3′. The handle sequence is the full transcribed gRNA sequence minus the spacer sequence. In general, the handle sequence for the gRNAs described in TABLE 6 is SEQ ID NO: 283. An exemplary reference number for the target, DMD, is NCBI Accession Number, NM_004006.3. In some embodiments, the gRNA may be represented by the full gRNA sequence with a transcript initiating guanine (G).

TABLE 6
Exemplary CasM.265466 gRNA Sequences

| | | | | | | | gRNA sequence |
|---|---|---|---|---|---|---|---|
| | Target | | | SEQ | | SEQ | (transcription |
| Target | (DMD | | Spacer | ID | | ID | initiating |
| Site | exon) | PAM | Sequence | NO: | gRNA Sequence (Handle + Spacer) | NO: | G) SEQ ID NO: |
| 1 | exon | CTTG | GUUUCUGUGA | 100 | ACAGCUUAUUUGGAAGCUGAAAUGUGAGGUU | 466 | 649 |
| | #53 | | UUUUCUUUUG | | UAUAACACUCACAAGAAUCCUGAAAAGGAUGC | | |
| | | | | | CAAACGUUUCUGUGAUUUUCUUUUG | | |
| 2 | exon | TCTG | UGAUUUUCUU | 101 | ACAGCUUAUUUGGAAGCUGAAAUGUGAGGUU | 467 | 650 |
| | #53 | | UUGGAUUGCA | | UAUAACACUCACAAGAAUCCUGAAAAGGAUGC | | |
| | | | | | CAAACUGAUUUUCUUUUGGAUUGCA | | |
| 3 | exon | TGTG | AUUUUCUUUU | 102 | ACAGCUUAUUUGGAAGCUGAAAUGUGAGGUU | 468 | 651 |
| | #53 | | GGAUUGCAUC | | UAUAACACUCACAAGAAUCCUGAAAAGGAUGC | | |
| | | | | | CAAACAUUUUCUUUUGGAUUGCAUC | | |
| 4 | exon | GAT | CAAUCCAAAA | 103 | ACAGCUUAUUUGGAAGCUGAAAUGUGAGGUU | 469 | 652 |
| | #53 | G | GAAAAUCACA | | UAUAACACUCACAAGAAUCCUGAAAAGGAUGC | | |
| | | | | | CAAACCAAUCCAAAAGAAAAUCACA | | |
| 5 | exon | AGT | GAUGCAAUCC | 104 | ACAGCUUAUUUGGAAGCUGAAAUGUGAGGUU | 470 | 653 |
| | #53 | A | AAAAGAAAA | | UAUAACACUCACAAGAAUCCUGAAAAGGAUGC | | |
| | | | U | | CAAACGAUGCAAUCCAAAAGAAAAU | | |
| 6 | exon | TATA | CAGUAGAUGC | 105 | ACAGCUUAUUUGGAAGCUGAAAUGUGAGGUU | 471 | 654 |
| | #53 | | AAUCCAAAAG | | UAUAACACUCACAAGAAUCCUGAAAAGGAUGC | | |
| | | | | | CAAACCAGUAGAUGCAAUCCAAAAG | | |
| 7 | exon | TTTG | GAUUGCAUCU | 106 | ACAGCUUAUUUGGAAGCUGAAAUGUGAGGUU | 472 | 655 |
| | #53 | | ACUGUAUAGG | | UAUAACACUCACAAGAAUCCUGAAAAGGAUGC | | |
| | | | | | CAAACGAUUGCAUCUACUGUAUAGG | | |
| 8 | exon | CCTA | UACAGUAGAU | 107 | ACAGCUUAUUUGGAAGCUGAAAUGUGAGGUU | 473 | 656 |
| | #53 | | GCAAUCCAAA | | UAUAACACUCACAAGAAUCCUGAAAAGGAUGC | | |
| | | | | | CAAACUACAGUAGAUGCAAUCCAAA | | |
| 9 | exon | ATTG | CAUCUACUGU | 108 | ACAGCUUAUUUGGAAGCUGAAAUGUGAGGUU | 474 | 657 |
| | #53 | | AUAGGGACCC | | UAUAACACUCACAAGAAUCCUGAAAAGGAUGC | | |
| | | | | | CAAACCAUCUACUGUAUAGGGACCC | | |
| 10 | exon | TCTA | CUGUAUAGGG | 109 | ACAGCUUAUUUGGAAGCUGAAAUGUGAGGUU | 475 | 658 |
| | #53 | | ACCCUCCUUC | | UAUAACACUCACAAGAAUCCUGAAAAGGAUGC | | |
| | | | | | CAAACCUGUAUAGGGACCCUCCUUC | | |
| 11 | exon | ACT | UAUAGGGACC | 110 | ACAGCUUAUUUGGAAGCUGAAAUGUGAGGUU | 476 | 659 |
| | #53 | G | CUCCUUCCAU | | UAUAACACUCACAAGAAUCCUGAAAAGGAUGC | | |
| | | | | | CAAACUAUAGGGACCCUCCUUCCAU | | |
| 12 | exon | CAT | GAAGGAGGG | 111 | ACAGCUUAUUUGGAAGCUGAAAUGUGAGGUU | 477 | 660 |
| | #53 | G | UCCCUAUACA | | UAUAACACUCACAAGAAUCCUGAAAAGGAUGC | | |
| | | | G | | CAAACGAAGGAGGGUCCCUAUACAG | | |
| 13 | exon | TGTA | UAGGGACCCU | 112 | ACAGCUUAUUUGGAAGCUGAAAUGUGAGGUU | 478 | 661 |
| | #53 | | CCUUCCAUGA | | UAUAACACUCACAAGAAUCCUGAAAAGGAUGC | | |
| | | | | | CAAACUAGGGACCCUCCUUCCAUGA | | |
| 14 | exon | TATA | GGGACCCUCC | 113 | ACAGCUUAUUUGGAAGCUGAAAUGUGAGGUU | 479 | 662 |
| | #53 | | UUCCAUGACU | | UAUAACACUCACAAGAAUCCUGAAAAGGAUGC | | |
| | | | | | CAAACGGGACCCUCCUUCCAUGACU | | |
| 15 | exon | CTTG | AGUCAUGGAA | 114 | ACAGCUUAUUUGGAAGCUGAAAUGUGAGGUU | 480 | 663 |
| | #53 | | GGAGGGUCCC | | UAUAACACUCACAAGAAUCCUGAAAAGGAUGC | | |
| | | | | | CAAACAGUCAUGGAAGGAGGGUCCC | | |
| 16 | exon | CAT | ACUCAAGCUU | 115 | ACAGCUUAUUUGGAAGCUGAAAUGUGAGGUU | 481 | 664 |
| | #53 | G | GGCUCUGGCC | | UAUAACACUCACAAGAAUCCUGAAAAGGAUGC | | |
| | | | | | CAAACACUCAAGCUUGGCUCUGGCC | | |
| 17 | exon | CTTA | GGACAGGCCA | 116 | ACAGCUUAUUUGGAAGCUGAAAUGUGAGGUU | 482 | 665 |
| | #53 | | GAGCCAAGCU | | UAUAACACUCACAAGAAUCCUGAAAAGGAUGC | | |
| | | | | | CAAACGGACAGGCCAGAGCCAAGCU | | |
| 18 | exon | CTTG | GCUCUGGCCU | 117 | ACAGCUUAUUUGGAAGCUGAAAUGUGAGGUU | 483 | 666 |
| | #53 | | GUCCUAAGAC | | UAUAACACUCACAAGAAUCCUGAAAAGGAUGC | | |
| | | | | | CAAACGCUCUGGCCUGUCCUAAGAC | | |
| 19 | exon | TCTG | GCCUGUCCUA | 118 | ACAGCUUAUUUGGAAGCUGAAAUGUGAGGUU | 484 | 667 |
| | #53 | | AGACCUGCUC | | UAUAACACUCACAAGAAUCCUGAAAAGGAUGC | | |
| | | | | | CAAACGCCUGUCCUAAGACCUGCUC | | |
| 20 | exon | GCT | AGCAGGUCUU | 119 | ACAGCUUAUUUGGAAGCUGAAAUGUGAGGUU | 485 | 668 |
| | #53 | G | AGGACAGGCC | | UAUAACACUCACAAGAAUCCUGAAAAGGAUGC | | |
| | | | | | CAAACAGCAGGUCUUAGGACAGGCC | | |
| 21 | exon | CCTG | UCCUAAGACC | 120 | ACAGCUUAUUUGGAAGCUGAAAUGUGAGGUU | 486 | 669 |
| | #53 | | UGCUCAGCUU | | UAUAACACUCACAAGAAUCCUGAAAAGGAUGC | | |
| | | | | | CAAACUCCUAAGACCUGCUCAGCUU | | |
| 22 | exon | CCTA | AGACCUGCUC | 121 | ACAGCUUAUUUGGAAGCUGAAAUGUGAGGUU | 487 | 670 |
| | #53 | | AGCUUCUUCC | | UAUAACACUCACAAGAAUCCUGAAAAGGAUGC | | |
| | | | | | CAAACAGACCUGCUCAGCUUCUUCC | | |
| 23 | exon | GCT | AGGAAGAAGC | 122 | ACAGCUUAUUUGGAAGCUGAAAUGUGAGGUU | 488 | 671 |
| | #53 | A | UGAGCAGGUC | | UAUAACACUCACAAGAAUCCUGAAAAGGAUGC | | |
| | | | | | CAAACAGGAAGAAGCUGAGCAGGUC | | |
| 24 | exon | CCTG | CUCAGCUUCU | 123 | ACAGCUUAUUUGGAAGCUGAAAUGUGAGGUU | 489 | 672 |
| | #53 | | UCCUUAGCUU | | UAUAACACUCACAAGAAUCCUGAAAAGGAUGC | | |
| | | | | | CAAACCUCAGCUUCUUCCUUAGCUU | | |
| 25 | exon | GCT | GAAGCUAAGG | 124 | ACAGCUUAUUUGGAAGCUGAAAUGUGAGGUU | 490 | 673 |
| | #53 | G | AAGAAGCUGA | | UAUAACACUCACAAGAAUCCUGAAAAGGAUGC | | |
| | | | | | CAAACGAAGCUAAGGAAGAAGCUGA | | |
| 26 | exon | AAT | GCUGGAAGCU | 125 | ACAGCUUAUUUGGAAGCUGAAAUGUGAGGUU | 491 | 674 |
| | #53 | G | AAGGAAGAA | | UAUAACACUCACAAGAAUCCUGAAAAGGAUGC | | |
| | | | G | | CAAACGCUGGAAGCUAAGGAAGAAG | | |
| 27 | exon | CTTA | GCUUCCAGCC | 126 | ACAGCUUAUUUGGAAGCUGAAAUGUGAGGUU | 492 | 675 |
| | #53 | | AUUGUGUUG | | UAUAACACUCACAAGAAUCCUGAAAAGGAUGC | | |
| | | | A | | CAAACGCUUCCAGCCAUUGUGUUGA | | |
| 28 | exon | GTTA | AAGGAUUCAA | 127 | ACAGCUUAUUUGGAAGCUGAAAUGUGAGGUU | 493 | 676 |
| | #53 | | CACAAUGGCU | | UAUAACACUCACAAGAAUCCUGAAAAGGAUGC | | |
| | | | | | CAAACAAGGAUUCAACACAAUGGCU | | |
| 29 | exon | AAT | UUAAAGGAU | 128 | ACAGCUUAUUUGGAAGCUGAAAUGUGAGGUU | 494 | 677 |
| | #53 | G | UCAACACAAU | | UAUAACACUCACAAGAAUCCUGAAAAGGAUGC | | |
| | | | G | | CAAACUUAAAGGAUUCAACACAAUG | | |
| 30 | exon | ATTG | UGUUGAAUCC | 129 | ACAGCUUAUUUGGAAGCUGAAAUGUGAGGUU | 495 | 678 |
| | #53 | | UUUAACAUUU | | UAUAACACUCACAAGAAUCCUGAAAAGGAUGC | | |
| | | | | | CAAACUGUUGAAUCCUUUAACAUUU | | |
| 31 | exon | TGTG | UUGAAUCCUU | 130 | ACAGCUUAUUUGGAAGCUGAAAUGUGAGGUU | 496 | 679 |
| | #53 | | UAACAUUUCA | | UAUAACACUCACAAGAAUCCUGAAAAGGAUGC | | |
| | | | | | CAAACUUGAAUCCUUUAACAUUUCA | | |
| 32 | exon | AAT | AAAUGUUAA | 131 | ACAGCUUAUUUGGAAGCUGAAAUGUGAGGUU | 497 | 680 |
| | #53 | G | AGGAUUCAAC | | UAUAACACUCACAAGAAUCCUGAAAAGGAUGC | | |
| | | | A | | CAAACAAAUGUUAAAGGAUUCAACA | | |
| 33 | exon | GTTG | AAUCCUUUAA | 132 | ACAGCUUAUUUGGAAGCUGAAAUGUGAGGUU | 498 | 681 |
| | #53 | | CAUUUCAUUC | | UAUAACACUCACAAGAAUCCUGAAAAGGAUGC | | |
| | | | | | CAAACAAUCCUUUAACAUUUCAUUC | | |
| 34 | exon | GTTG | AAUGAAAUG | 133 | ACAGCUUAUUUGGAAGCUGAAAUGUGAGGUU | 499 | 682 |
| | #53 | | UUAAAGGAU | | UAUAACACUCACAAGAAUCCUGAAAAGGAUGC | | |
| | | | UC | | CAAACAAUGAAAUGUUAAAGGAUUC | | |
| 35 | exon | TTTA | ACAUUUCAUU | 134 | ACAGCUUAUUUGGAAGCUGAAAUGUGAGGUU | 500 | 683 |
| | #53 | | CAACUGUUGC | | UAUAACACUCACAAGAAUCCUGAAAAGGAUGC | | |
| | | | | | CAAACACAUUUCAUUCAACUGUUGC | | |
| 36 | exon | ACT | UUGCCUCCGG | 135 | ACAGCUUAUUUGGAAGCUGAAAUGUGAGGUU | 501 | 684 |
| | #53 | G | UUCUGAAGGU | | UAUAACACUCACAAGAAUCCUGAAAAGGAUGC | | |
| | | | | | CAAACUUGCCUCCGGUUCUGAAGGU | | |
| 37 | exon | GTTG | CCUCCGGUUC | 136 | ACAGCUUAUUUGGAAGCUGAAAUGUGAGGUU | 502 | 685 |
| | #53 | | UGAAGGUGU | | UAUAACACUCACAAGAAUCCUGAAAAGGAUGC | | |
| | | | U | | CAAACCCUCCGGUUCUGAAGGUGUU | | |
| 38 | exon | AGT | CAAGAACACC | 137 | ACAGCUUAUUUGGAAGCUGAAAUGUGAGGUU | 503 | 686 |
| | #53 | A | UUCAGAACCG | | UAUAACACUCACAAGAAUCCUGAAAAGGAUGC | | |
| | | | | | CAAACCAAGAACACCUUCAGAACCG | | |
| 39 | exon | TCTG | AAGGUGUUCU | 138 | ACAGCUUAUUUGGAAGCUGAAAUGUGAGGUU | 504 | 687 |
| | #53 | | UGUACUUCAU | | UAUAACACUCACAAGAAUCCUGAAAAGGAUGC | | |
| | | | | | CAAACAAGGUGUUCUUGUACUUCAU | | |
| 40 | exon | GAT | AAGUACAAGA | 139 | ACAGCUUAUUUGGAAGCUGAAAUGUGAGGUU | 505 | 688 |
| | #53 | G | ACACCUUCAG | | UAUAACACUCACAAGAAUCCUGAAAAGGAUGC | | |
| | | | | | CAAACAAGUACAAGAACACCUUCAG | | |
| 41 | exon | GGT | UUCUUGUACU | 140 | ACAGCUUAUUUGGAAGCUGAAAUGUGAGGUU | 506 | 689 |
| | #53 | G | UCAUCCCACU | | UAUAACACUCACAAGAAUCCUGAAAAGGAUGC | | |
| | | | | | CAAACUUCUUGUACUUCAUCCCACU | | |
| 42 | exon | AGT | GGAUGAAGU | 141 | ACAGCUUAUUUGGAAGCUGAAAUGUGAGGUU | 507 | 690 |
| | #53 | G | ACAAGAACAC | | UAUAACACUCACAAGAAUCCUGAAAAGGAUGC | | |
| | | | C | | CAAACGGAUGAAGUACAAGAACACC | | |
| 43 | exon | CTTG | UACUUCAUCC | 142 | ACAGCUUAUUUGGAAGCUGAAAUGUGAGGUU | 508 | 691 |
| | #53 | | CACUGAUUCU | | UAUAACACUCACAAGAAUCCUGAAAAGGAUGC | | |
| | | | | | CAAACUACUUCAUCCCACUGAUUCU | | |
| 44 | exon | TGTA | CUUCAUCCCA | 143 | ACAGCUUAUUUGGAAGCUGAAAUGUGAGGUU | 509 | 692 |
| | #53 | | CUGAUUCUGA | | UAUAACACUCACAAGAAUCCUGAAAAGGAUGC | | |
| | | | | | CAAACCUUCAUCCCACUGAUUCUGA | | |
| 45 | exon | GCT | GAACAAUCAU | 144 | ACAGCUUAUUUGGAAGCUGAAAUGUGAGGUU | 510 | 693 |
| | #52 | A | UACGGAUCGA | | UAUAACACUCACAAGAAUCCUGAAAAGGAUGC | | |
| | | | | | CAAACGAACAAUCAUUACGGAUCGA | | |
| 46 | exon | CGT | AUGAUUGUUC | 145 | ACAGCUUAUUUGGAAGCUGAAAUGUGAGGUU | 511 | 694 |
| | #52 | A | UAGCCUCUUG | | UAUAACACUCACAAGAAUCCUGAAAAGGAUGC | | |
| | | | | | CAAACAUGAUUGUUCUAGCCUCUUG | | |
| 47 | exon | AAT | AUUGUUCUAG | 146 | ACAGCUUAUUUGGAAGCUGAAAUGUGAGGUU | 512 | 695 |
| | #52 | G | CCUCUUGAUU | | UAUAACACUCACAAGAAUCCUGAAAAGGAUGC | | |
| | | | | | CAAACAUUGUUCUAGCCUCUUGAUU | | |
| 48 | exon | ATTG | UUCUAGCCUC | 147 | ACAGCUUAUUUGGAAGCUGAAAUGUGAGGUU | 513 | 696 |
| | #52 | | UUGAUUGCUG | | UAUAACACUCACAAGAAUCCUGAAAAGGAUGC | | |
| | | | | | CAAACUUCUAGCCUCUUGAUUGCUG | | |
| 49 | exon | TCTA | GCCUCUUGAU | 148 | ACAGCUUAUUUGGAAGCUGAAAUGUGAGGUU | 514 | 697 |
| | #52 | | UGCUGGUCUU | | UAUAACACUCACAAGAAUCCUGAAAAGGAUGC | | |
| | | | | | CAAACGCCUCUUGAUUGCUGGUCUU | | |
| 50 | exon | CTTG | AUUGCUGGUC | 149 | ACAGCUUAUUUGGAAGCUGAAAUGUGAGGUU | 515 | 698 |
| | #52 | | UUGUUUUUCA | | UAUAACACUCACAAGAAUCCUGAAAAGGAUGC | | |
| | | | | | CAAACAUUGCUGGUCUUGUUUUUCA | | |
| 51 | exon | TTTG | AAAAACAAGA | 150 | ACAGCUUAUUUGGAAGCUGAAAUGUGAGGUU | 516 | 699 |
| | #52 | | CCAGCAAUCA | | UAUAACACUCACAAGAAUCCUGAAAAGGAUGC | | |
| | | | | | CAAACAAAAACAAGACCAGCAAUCA | | |
| 52 | exon | ATTG | CUGGUCUUGU | 151 | ACAGCUUAUUUGGAAGCUGAAAUGUGAGGUU | 517 | 700 |
| | #52 | | UUUUCAAAUU | | UAUAACACUCACAAGAAUCCUGAAAAGGAUGC | | |
| | | | | | CAAACCUGGUCUUGUUUUUCAAAUU | | |
| 53 | exon | GCT | GUCUUGUUUU | 152 | ACAGCUUAUUUGGAAGCUGAAAUGUGAGGUU | 518 | 701 |
| | #52 | G | UCAAAUUUUG | | UAUAACACUCACAAGAAUCCUGAAAAGGAUGC | | |
| | | | | | CAAACGUCUUGUUUUUCAAAUUUUG | | |
| 54 | exon | CTTG | UUUUUCAAAU | 153 | ACAGCUUAUUUGGAAGCUGAAAUGUGAGGUU | 519 | 702 |
| | #52 | | UUUGGGCAGC | | UAUAACACUCACAAGAAUCCUGAAAAGGAUGC | | |
| | | | | | CAAACUUUUUCAAAUUUUGGGCAGC | | |
| 55 | exon | GCT | CCCAAAAUUU | 154 | ACAGCUUAUUUGGAAGCUGAAAUGUGAGGUU | 520 | 703 |
| | #52 | G | GAAAAACAAG | | UAUAACACUCACAAGAAUCCUGAAAAGGAUGC | | |
| | | | | | CAAACCCCAAAAUUUGAAAAACAAG | | |
| 56 | exon | ATTA | CCGCUGCCCA | 155 | ACAGCUUAUUUGGAAGCUGAAAUGUGAGGUU | 521 | 704 |
| | #52 | | AAAUUUGAA | | UAUAACACUCACAAGAAUCCUGAAAAGGAUGC | | |
| | | | A | | CAAACCCGCUGCCCAAAAUUUGAAA | | |
| 57 | exon | TTTG | GGCAGCGGUA | 156 | ACAGCUUAUUUGGAAGCUGAAAUGUGAGGUU | 522 | 705 |
| | #52 | | AUGAGUUCUU | | UAUAACACUCACAAGAAUCCUGAAAAGGAUGC | | |
| | | | | | CAAACGGCAGCGGUAAUGAGUUCUU | | |
| 58 | exon | GTTG | GAAGAACUCA | 157 | ACAGCUUAUUUGGAAGCUGAAAUGUGAGGUU | 523 | 706 |
| | #52 | | UUACCGCUGC | | UAUAACACUCACAAGAAUCCUGAAAAGGAUGC | | |
| | | | | | CAAACGAAGAACUCAUUACCGCUGC | | |
| 59 | exon | GGT | AUGAGUUCUU | 158 | ACAGCUUAUUUGGAAGCUGAAAUGUGAGGUU | 524 | 707 |
| | #52 | A | CCAACUGGGG | | UAUAACACUCACAAGAAUCCUGAAAAGGAUGC | | |
| | | | | | CAAACAUGAGUUCUUCCAACUGGGG | | |
| 60 | exon | AAT | AGUUCUUCCA | 159 | ACAGCUUAUUUGGAAGCUGAAAUGUGAGGUU | 525 | 708 |
| | #52 | G | ACUGGGGACG | | UAUAACACUCACAAGAAUCCUGAAAAGGAUGC | | |
| | | | | | CAAACAGUUCUUCCAACUGGGGACG | | |
| 61 | exon | TTTG | GAACAGAGGC | 160 | ACAGCUUAUUUGGAAGCUGAAAUGUGAGGUU | 526 | 709 |
| | #52 | | GUCCCCAGUU | | UAUAACACUCACAAGAAUCCUGAAAAGGAUGC | | |
| | | | | | CAAACGAACAGAGGCGUCCCCAGUU | | |
| 62 | exon | ACT | GGGACGCCUC | 161 | ACAGCUUAUUUGGAAGCUGAAAUGUGAGGUU | 527 | 710 |
| | #52 | G | UGUUCCAAAU | | UAUAACACUCACAAGAAUCCUGAAAAGGAUGC | | |
| | | | | | CAAACGGGACGCCUCUGUUCCAAAU | | |
| 63 | exon | AAT | CAGGAUUUGG | 162 | ACAGCUUAUUUGGAAGCUGAAAUGUGAGGUU | 528 | 711 |
| | #52 | G | AACAGAGGCG | | UAUAACACUCACAAGAAUCCUGAAAAGGAUGC | | |
| | | | | | CAAACCAGGAUUUGGAACAGAGGCG | | |
| 64 | exon | TCTG | UUCCAAAUCC | 163 | ACAGCUUAUUUGGAAGCUGAAAUGUGAGGUU | 529 | 712 |
| | #52 | | UGCAUUGUUG | | UAUAACACUCACAAGAAUCCUGAAAAGGAUGC | | |
| | | | | | CAAACUUCCAAAUCCUGCAUUGUUG | | |
| 65 | exon | TCTG | CUUGAUGAUC | 164 | ACAGCUUAUUUGGAAGCUGAAAUGUGAGGUU | 530 | 713 |
| | #51 | | AUCUCGUUGA | | UAUAACACUCACAAGAAUCCUGAAAAGGAUGC | | |
| | | | | | CAAACCUUGAUGAUCAUCUCGUUGA | | |
| 66 | exon | CTTG | AUGAUCAUCU | 165 | ACAGCUUAUUUGGAAGCUGAAAUGUGAGGUU | 531 | 714 |
| | #51 | | CGUUGAUAUC | | UAUAACACUCACAAGAAUCCUGAAAAGGAUGC | | |
| | | | | | CAAACAUGAUCAUCUCGUUGAUAUC | | |
| 67 | exon | GAT | UCAACGAGAU | 166 | ACAGCUUAUUUGGAAGCUGAAAUGUGAGGUU | 532 | 715 |
| | #51 | A | GAUCAUCAAG | | UAUAACACUCACAAGAAUCCUGAAAAGGAUGC | | |
| | | | | | CAAACUCAACGAGAUGAUCAUCAAG | | |
| 68 | exon | GAT | AUCAUCUCGU | 167 | ACAGCUUAUUUGGAAGCUGAAAUGUGAGGUU | 533 | 716 |
| | #51 | G | UGAUAUCCUC | | UAUAACACUCACAAGAAUCCUGAAAAGGAUGC | | |
| | | | | | CAAACAUCAUCUCGUUGAUAUCCUC | | |
| 69 | exon | CTTG | AGGAUAUCAA | 168 | ACAGCUUAUUUGGAAGCUGAAAUGUGAGGUU | 534 | 717 |
| | #51 | | CGAGAUGAUC | | UAUAACACUCACAAGAAUCCUGAAAAGGAUGC | | |
| | | | | | CAAACAGGAUAUCAACGAGAUGAUC | | |
| 70 | exon | GGT | ACCUUGAGGA | 169 | ACAGCUUAUUUGGAAGCUGAAAUGUGAGGUU | 535 | 718 |
| | #51 | G | UAUCAACGAG | | UAUAACACUCACAAGAAUCCUGAAAAGGAUGC | | |
| | | | | | CAAACACCUUGAGGAUAUCAACGAG | | |
| 71 | exon | GTTG | AUAUCCUCAA | 170 | ACAGCUUAUUUGGAAGCUGAAAUGUGAGGUU | 536 | 719 |
| | #51 | | GGUCACCCAC | | UAUAACACUCACAAGAAUCCUGAAAAGGAUGC | | |
| | | | | | CAAACAUAUCCUCAAGGUCACCCAC | | |
| 72 | exon | GGT | GGUGACCUUG | 171 | ACAGCUUAUUUGGAAGCUGAAAUGUGAGGUU | 537 | 720 |
| | #51 | G | AGGAUAUCAA | | UAUAACACUCACAAGAAUCCUGAAAAGGAUGC | | |
| | | | | | CAAACGGUGACCUUGAGGAUAUCAA | | |
| 73 | exon | GAT | UCCUCAAGGU | 172 | ACAGCUUAUUUGGAAGCUGAAAUGUGAGGUU | 538 | 721 |
| | #51 | A | CACCCACCAU | | UAUAACACUCACAAGAAUCCUGAAAAGGAUGC | | |
| | | | | | CAAACUCCUCAAGGUCACCCACCAU | | |
| 74 | exon | GAT | GUGGGUGACC | 173 | ACAGCUUAUUUGGAAGCUGAAAUGUGAGGUU | 539 | 722 |
| | #51 | G | UUGAGGAUA | | UAUAACACUCACAAGAAUCCUGAAAAGGAUGC | | |
| | | | U | | CAAACGUGGGUGACCUUGAGGAUAU | | |
| 75 | exon | GGT | AUGGUGGGU | 174 | ACAGCUUAUUUGGAAGCUGAAAUGUGAGGUU | 540 | 723 |
| | #51 | G | GACCUUGAGG | | UAUAACACUCACAAGAAUCCUGAAAAGGAUGC | | |
| | | | A | | CAAACAUGGUGGGUGACCUUGAGGA | | |
| 76 | exon | TATA | AAAUCACAGA | 175 | ACAGCUUAUUUGGAAGCUGAAAUGUGAGGUU | 541 | 724 |
| | #51 | | GGGUGAUGG | | UAUAACACUCACAAGAAUCCUGAAAAGGAUGC | | |
| | | | U | | CAAACAAAUCACAGAGGGUGAUGGU | | |
| 77 | exon | GTTA | UAAAAUCACA | 176 | ACAGCUUAUUUGGAAGCUGAAAUGUGAGGUU | 542 | 725 |
| | #51 | | GAGGGUGAU | | UAUAACACUCACAAGAAUCCUGAAAAGGAUGC | | |
| | | | G | | CAAACUAAAAUCACAGAGGGUGAUG | | |
| 78 | exon | CTTG | AUCAAGUUAU | 177 | ACAGCUUAUUUGGAAGCUGAAAUGUGAGGUU | 543 | 726 |
| | #51 | | AAAAUCACAG | | UAUAACACUCACAAGAAUCCUGAAAAGGAUGC | | |
| | | | | | CAAACAUCAAGUUAUAAAAUCACAG | | |
| 79 | exon | TGTG | AUUUUAUAAC | 178 | ACAGCUUAUUUGGAAGCUGAAAUGUGAGGUU | 544 | 727 |
| | #51 | | UUGAUCAAGC | | UAUAACACUCACAAGAAUCCUGAAAAGGAUGC | | |
| | | | | | CAAACAUUUUAUAACUUGAUCAAGC | | |
| 80 | exon | TCTG | CUUGAUCAAG | 179 | ACAGCUUAUUUGGAAGCUGAAAUGUGAGGUU | 545 | 728 |
| | #51 | | UUAUAAAAUC | | UAUAACACUCACAAGAAUCCUGAAAAGGAUGC | | |
| | | | | | CAAACCUUGAUCAAGUUAUAAAAUC | | |
| 81 | exon | TTTA | UAACUUGAUC | 180 | ACAGCUUAUUUGGAAGCUGAAAUGUGAGGUU | 546 | 729 |
| | #51 | | AAGCAGAGAA | | UAUAACACUCACAAGAAUCCUGAAAAGGAUGC | | |
| | | | | | CAAACUAACUUGAUCAAGCAGAGAA | | |
| 82 | exon | TATA | ACUUGAUCAA | 181 | ACAGCUUAUUUGGAAGCUGAAAUGUGAGGUU | 547 | 730 |
| | #51 | | GCAGAGAAAG | | UAUAACACUCACAAGAAUCCUGAAAAGGAUGC | | |
| | | | | | CAAACACUUGAUCAAGCAGAGAAAG | | |
| 83 | exon | CTTG | AUCAAGCAGA | 182 | ACAGCUUAUUUGGAAGCUGAAAUGUGAGGUU | 548 | 731 |
| | #51 | | GAAAGCCAGU | | UAUAACACUCACAAGAAUCCUGAAAAGGAUGC | | |
| | | | | | CAAACAUCAAGCAGAGAAAGCCAGU | | |
| 84 | exon | ACT | GCUUUCUCUG | 183 | ACAGCUUAUUUGGAAGCUGAAAUGUGAGGUU | 549 | 732 |
| | #51 | G | CUUGAUCAAG | | UAUAACACUCACAAGAAUCCUGAAAAGGAUGC | | |
| | | | | | CAAACGCUUUCUCUGCUUGAUCAAG | | |
| 85 | exon | CTTA | CCGACUGGCU | 184 | ACAGCUUAUUUGGAAGCUGAAAUGUGAGGUU | 550 | 733 |
| | #51 | | UUCUCUGCUU | | UAUAACACUCACAAGAAUCCUGAAAAGGAUGC | | |
| | | | | | CAAACCCGACUGGCUUUCUCUGCUU | | |
| 86 | exon | CTTG | GACAGAACUU | 185 | ACAGCUUAUUUGGAAGCUGAAAUGUGAGGUU | 551 | 734 |
| | #51 | | ACCGACUGGC | | UAUAACACUCACAAGAAUCCUGAAAAGGAUGC | | |
| | | | | | CAAACGACAGAACUUACCGACUGGC | | |
| 87 | exon | GGT | AGUUCUGUCC | 186 | ACAGCUUAUUUGGAAGCUGAAAUGUGAGGUU | 552 | 735 |
| | #51 | A | AAGCCCGGUU | | UAUAACACUCACAAGAAUCCUGAAAAGGAUGC | | |
| | | | | | CAAACAGUUCUGUCCAAGCCCGGUU | | |
| 88 | exon | TCTG | UCCAAGCCCG | 187 | ACAGCUUAUUUGGAAGCUGAAAUGUGAGGUU | 553 | 736 |
| | #51 | | GUUGAAAUCU | | UAUAACACUCACAAGAAUCCUGAAAAGGAUGC | | |
| | | | | | CAAACUCCAAGCCCGGUUGAAAUCU | | |
| 89 | exon | TCTG | GCAGAUUUCA | 188 | ACAGCUUAUUUGGAAGCUGAAAUGUGAGGUU | 554 | 737 |
| | #51 | | ACCGGGCUUG | | UAUAACACUCACAAGAAUCCUGAAAAGGAUGC | | |
| | | | | | CAAACGCAGAUUUCAACCGGGCUUG | | |
| 90 | exon | CCTG | CUCUGGCAGA | 189 | ACAGCUUAUUUGGAAGCUGAAAUGUGAGGUU | 555 | 738 |
| | #51 | | UUUCAACCGG | | UAUAACACUCACAAGAAUCCUGAAAAGGAUGC | | |
| | | | | | CAAACCUCUGGCAGAUUUCAACCGG | | |
| 91 | exon | GTTG | AAAUCUGCCA | 190 | ACAGCUUAUUUGGAAGCUGAAAUGUGAGGUU | 556 | 739 |
| | #51 | | GAGCAGGUAC | | UAUAACACUCACAAGAAUCCUGAAAAGGAUGC | | |
| | | | | | CAAACAAAUCUGCCAGAGCAGGUAC | | |
| 92 | exon | GGT | CCUGCUCUGG | 191 | ACAGCUUAUUUGGAAGCUGAAAUGUGAGGUU | 557 | 740 |
| | #51 | A | CAGAUUUCAA | | UAUAACACUCACAAGAAUCCUGAAAAGGAUGC | | |
| | | | | | CAAACCCUGCUCUGGCAGAUUUCAA | | |
| 93 | exon | TCTG | CCAGAGCAGG | 192 | ACAGCUUAUUUGGAAGCUGAAAUGUGAGGUU | 558 | 741 |
| | #51 | | UACCUCCAAC | | UAUAACACUCACAAGAAUCCUGAAAAGGAUGC | | |
| | | | | | CAAACCCAGAGCAGGUACCUCCAAC | | |
| 94 | exon | GTTG | GAGGUACCUG | 193 | ACAGCUUAUUUGGAAGCUGAAAUGUGAGGUU | 559 | 742 |
| | #51 | | CUCUGGCAGA | | UAUAACACUCACAAGAAUCCUGAAAAGGAUGC | | |
| | | | | | CAAACGAGGUACCUGCUCUGGCAGA | | |
| 95 | exon | GAT | UUGGAGGUAC | 194 | ACAGCUUAUUUGGAAGCUGAAAUGUGAGGUU | 560 | 743 |
| | #51 | G | CUGCUCUGGC | | UAUAACACUCACAAGAAUCCUGAAAAGGAUGC | | |
| | | | | | CAAACUUGGAGGUACCUGCUCUGGC | | |
| 96 | exon | CTTG | AUGUUGGAG | 195 | ACAGCUUAUUUGGAAGCUGAAAUGUGAGGUU | 561 | 744 |
| | #51 | | GUACCUGCUC | | UAUAACACUCACAAGAAUCCUGAAAAGGAUGC | | |
| | | | U | | CAAACAUGUUGGAGGUACCUGCUCU | | |
| 97 | exon | GGT | CCUCCAACAU | 196 | ACAGCUUAUUUGGAAGCUGAAAUGUGAGGUU | 562 | 745 |
| | #51 | A | CAAGGAAGAU | | UAUAACACUCACAAGAAUCCUGAAAAGGAUGC | | |
| | | | | | CAAACCCUCCAACAUCAAGGAAGAU | | |
| 98 | exon | AAT | CCAUCUUCCU | 197 | ACAGCUUAUUUGGAAGCUGAAAUGUGAGGUU | 563 | 746 |
| | #51 | G | UGAUGUUGG | | UAUAACACUCACAAGAAUCCUGAAAAGGAUGC | | |
| | | | A | | CAAACCCAUCUUCCUUGAUGUUGGA | | |
| 99 | exon | ACT | GAAAUGCCAU | 198 | ACAGCUUAUUUGGAAGCUGAAAUGUGAGGUU | 564 | 747 |
| | #51 | A | CUUCCUUGAU | | UAUAACACUCACAAGAAUCCUGAAAAGGAUGC | | |
| | | | | | CAAACGAAAUGCCAUCUUCCUUGAU | | |
| 100 | exon | GAT | GCAUUUCUAG | 199 | ACAGCUUAUUUGGAAGCUGAAAUGUGAGGUU | 565 | 748 |
| | #51 | G | UUUGGAGAU | | UAUAACACUCACAAGAAUCCUGAAAAGGAUGC | | |
| | | | G | | CAAACGCAUUUCUAGUUUGGAGAUG | | |
| 101 | exon | ACT | CCAUCUCCAA | 200 | ACAGCUUAUUUGGAAGCUGAAAUGUGAGGUU | 566 | 749 |
| | #51 | G | ACUAGAAAUG | | UAUAACACUCACAAGAAUCCUGAAAAGGAUGC | | |
| | | | | | CAAACCCAUCUCCAAACUAGAAAUG | | |
| 102 | exon | TCTA | GUUUGGAGA | 201 | ACAGCUUAUUUGGAAGCUGAAAUGUGAGGUU | 567 | 750 |
| | #51 | | UGGCAGUUUC | | UAUAACACUCACAAGAAUCCUGAAAAGGAUGC | | |
| | | | C | | CAAACGUUUGGAGAUGGCAGUUUCC | | |
| 103 | exon | TTTG | GAGAUGGCAG | 202 | ACAGCUUAUUUGGAAGCUGAAAUGUGAGGUU | 568 | 751 |
| | #51 | | UUUCCUUAGU | | UAUAACACUCACAAGAAUCCUGAAAAGGAUGC | | |
| | | | | | CAAACGAGAUGGCAGUUUCCUUAGU | | |
| 104 | exon | ACT | AGGAAACUGC | 203 | ACAGCUUAUUUGGAAGCUGAAAUGUGAGGUU | 569 | 752 |
| | #51 | A | CAUCUCCAAA | | UAUAACACUCACAAGAAUCCUGAAAAGGAUGC | | |
| | | | | | CAAACAGGAAACUGCCAUCUCCAAA | | |
| 105 | exon | GTTA | CUAAGGAAAC | 204 | ACAGCUUAUUUGGAAGCUGAAAUGUGAGGUU | 570 | 753 |
| | #51 | | UGCCAUCUCC | | UAUAACACUCACAAGAAUCCUGAAAAGGAUGC | | |
| | | | | | CAAACCUAAGGAAACUGCCAUCUCC | | |
| 106 | exon | GAT | GCAGUUUCCU | 205 | ACAGCUUAUUUGGAAGCUGAAAUGUGAGGUU | 571 | 754 |
| | #51 | G | UAGUAACCAC | | UAUAACACUCACAAGAAUCCUGAAAAGGAUGC | | |
| | | | | | CAAACGCAGUUUCCUUAGUAACCAC | | |
| 107 | exon | TGTG | GUUACUAAGG | 206 | ACAGCUUAUUUGGAAGCUGAAAUGUGAGGUU | 572 | 755 |
| | #51 | | AAACUGCCAU | | UAUAACACUCACAAGAAUCCUGAAAAGGAUGC | | |
| | | | | | CAAACGUUACUAAGGAAACUGCCAU | | |
| 108 | exon | CCTG | UGGUUACUAA | 207 | ACAGCUUAUUUGGAAGCUGAAAUGUGAGGUU | 573 | 756 |
| | #51 | | GGAAACUGCC | | UAUAACACUCACAAGAAUCCUGAAAAGGAUGC | | |
| | | | | | CAAACUGGUUACUAAGGAAACUGCC | | |
| 109 | exon | CTTA | GUAACCACAG | 208 | ACAGCUUAUUUGGAAGCUGAAAUGUGAGGUU | 574 | 757 |
| | #51 | | GUUGUGUCAC | | UAUAACACUCACAAGAAUCCUGAAAAGGAUGC | | |
| | | | | | CAAACGUAACCACAGGUUGUGUCAC | | |
| 110 | exon | GGT | ACACAACCUG | 209 | ACAGCUUAUUUGGAAGCUGAAAUGUGAGGUU | 575 | 758 |
| | #51 | G | UGGUUACUAA | | UAUAACACUCACAAGAAUCCUGAAAAGGAUGC | | |
| | | | | | CAAACACACAACCUGUGGUUACUAA | | |
| 111 | exon | AGT | ACCACAGGUU | 210 | ACAGCUUAUUUGGAAGCUGAAAUGUGAGGUU | 576 | 759 |
| | #51 | A | GUGUCACCAG | | UAUAACACUCACAAGAAUCCUGAAAAGGAUGC | | |
| | | | | | CAAACACCACAGGUUGUGUCACCAG | | |
| 112 | exon | TCTG | GUGACACAAC | 211 | ACAGCUUAUUUGGAAGCUGAAAUGUGAGGUU | 577 | 760 |
| | #51 | | CUGUGGUUAC | | UAUAACACUCACAAGAAUCCUGAAAAGGAUGC | | |
| | | | | | CAAACGUGACACAACCUGUGGUUAC | | |
| 113 | exon | GTTA | CUCUGGUGAC | 212 | ACAGCUUAUUUGGAAGCUGAAAUGUGAGGUU | 578 | 761 |
| | #51 | | ACAACCUGUG | | UAUAACACUCACAAGAAUCCUGAAAAGGAUGC | | |
| | | | | | CAAACCUCUGGUGACACAACCUGUG | | |
| 114 | exon | ACT | UUACUCUGGU | 213 | ACAGCUUAUUUGGAAGCUGAAAUGUGAGGUU | 579 | 762 |
| | #51 | G | GACACAACCU | | UAUAACACUCACAAGAAUCCUGAAAAGGAUGC | | |
| | | | | | CAAACUUACUCUGGUGACACAACCU | | |
| 115 | exon | GTTG | UGUCACCAGA | 214 | ACAGCUUAUUUGGAAGCUGAAAUGUGAGGUU | 580 | 763 |
| | #51 | | GUAACAGUCU | | UAUAACACUCACAAGAAUCCUGAAAAGGAUGC | | |
| | | | | | CAAACUGUCACCAGAGUAACAGUCU | | |
| 116 | exon | TGTG | UCACCAGAGU | 215 | ACAGCUUAUUUGGAAGCUGAAAUGUGAGGUU | 581 | 764 |
| | #51 | | AACAGUCUGA | | UAUAACACUCACAAGAAUCCUGAAAAGGAUGC | | |
| | | | | | CAAACUCACCAGAGUAACAGUCUGA | | |
| 117 | exon | CCTA | CUCAGACUGU | 216 | ACAGCUUAUUUGGAAGCUGAAAUGUGAGGUU | 582 | 765 |
| | #51 | | UACUCUGGUG | | UAUAACACUCACAAGAAUCCUGAAAAGGAUGC | | |
| | | | | | CAAACCUCAGACUGUUACUCUGGUG | | |
| 118 | exon | TCTG | CAAACAGCUG | 217 | ACAGCUUAUUUGGAAGCUGAAAUGUGAGGUU | 583 | 766 |
| | #45 | | UCAGACAGAA | | UAUAACACUCACAAGAAUCCUGAAAAGGAUGC | | |
| | | | | | CAAACCAAACAGCUGUCAGACAGAA | | |
| 119 | exon | TCTG | UCUGACAGCU | 218 | ACAGCUUAUUUGGAAGCUGAAAUGUGAGGUU | 584 | 767 |
| | #45 | | GUUUGCAGAC | | UAUAACACUCACAAGAAUCCUGAAAAGGAUGC | | |
| | | | | | CAAACUCUGACAGCUGUUUGCAGAC | | |
| 120 | exon | TCTG | ACAGCUGUUU | 219 | ACAGCUUAUUUGGAAGCUGAAAUGUGAGGUU | 585 | 768 |
| | #45 | | GCAGACCUCC | | UAUAACACUCACAAGAAUCCUGAAAAGGAUGC | | |
| | | | | | CAAACACAGCUGUUUGCAGACCUCC | | |
| 121 | exon | GCT | UUUGCAGACC | 220 | ACAGCUUAUUUGGAAGCUGAAAUGUGAGGUU | 586 | 769 |
| | #45 | G | UCCUGCCACC | | UAUAACACUCACAAGAAUCCUGAAAAGGAUGC | | |
| | | | | | CAAACUUUGCAGACCUCCUGCCACC | | |
| 122 | exon | GGT | GCAGGAGGUC | 221 | ACAGCUUAUUUGGAAGCUGAAAUGUGAGGUU | 587 | 770 |
| | #45 | G | UGCAAACAGC | | UAUAACACUCACAAGAAUCCUGAAAAGGAUGC | | |
| | | | | | CAAACGCAGGAGGUCUGCAAACAGC | | |
| 123 | exon | TTTG | CAGACCUCCU | 222 | ACAGCUUAUUUGGAAGCUGAAAUGUGAGGUU | 588 | 771 |
| | #45 | | GCCACCGCAG | | UAUAACACUCACAAGAAUCCUGAAAAGGAUGC | | |
| | | | | | CAAACCAGACCUCCUGCCACCGCAG | | |
| 124 | exon | TCTG | CGGUGGCAGG | 223 | ACAGCUUAUUUGGAAGCUGAAAUGUGAGGUU | 589 | 772 |
| | #45 | | AGGUCUGCAA | | UAUAACACUCACAAGAAUCCUGAAAAGGAUGC | | |
| | | | | | CAAACCGGUGGCAGGAGGUCUGCAA | | |
| 125 | exon | CCTG | AAUCUGCGGU | 224 | ACAGCUUAUUUGGAAGCUGAAAUGUGAGGUU | 590 | 773 |
| | #45 | | GGCAGGAGGU | | UAUAACACUCACAAGAAUCCUGAAAAGGAUGC | | |
| | | | | | CAAACAAUCUGCGGUGGCAGGAGGU | | |
| 126 | exon | CCTG | CCACCGCAGA | 225 | ACAGCUUAUUUGGAAGCUGAAAUGUGAGGUU | 591 | 774 |
| | #45 | | UUCAGGCUUC | | UAUAACACUCACAAGAAUCCUGAAAAGGAUGC | | |
| | | | | | CAAACCCACCGCAGAUUCAGGCUUC | | |
| 127 | exon | ATTG | GGAAGCCUGA | 226 | ACAGCUUAUUUGGAAGCUGAAAUGUGAGGUU | 592 | 775 |
| | #45 | | AUCUGCGGUG | | UAUAACACUCACAAGAAUCCUGAAAAGGAUGC | | |
| | | | | | CAAACGGAAGCCUGAAUCUGCGGUG | | |
| 128 | exon | TCTA | CAGGAAAAAU | 227 | ACAGCUUAUUUGGAAGCUGAAAUGUGAGGUU | 593 | 776 |
| | #45 | | UGGGAAGCCU | | UAUAACACUCACAAGAAUCCUGAAAAGGAUGC | | |
| | | | | | CAAACCAGGAAAAAUUGGGAAGCCU | | |
| 129 | exon | AGT | UUCUACAGGA | 228 | ACAGCUUAUUUGGAAGCUGAAAUGUGAGGUU | 594 | 777 |
| | #45 | A | AAAAUUGGG | | UAUAACACUCACAAGAAUCCUGAAAAGGAUGC | | |
| | | | A | | CAAACUUCUACAGGAAAAAUUGGGA | | |
| 130 | exon | GAT | CCAGUAUUCU | 229 | ACAGCUUAUUUGGAAGCUGAAAUGUGAGGUU | 595 | 778 |
| | #45 | G | ACAGGAAAAA | | UAUAACACUCACAAGAAUCCUGAAAAGGAUGC | | |
| | | | | | CAAACCCAGUAUUCUACAGGAAAAA | | |
| 131 | exon | CCTG | UAGAAUACUG | 230 | ACAGCUUAUUUGGAAGCUGAAAUGUGAGGUU | 596 | 779 |
| | #45 | | GCAUCUGUUU | | UAUAACACUCACAAGAAUCCUGAAAAGGAUGC | | |
| | | | | | CAAACUAGAAUACUGGCAUCUGUUU | | |
| 132 | exon | TGTA | GAAUACUGGC | 231 | ACAGCUUAUUUGGAAGCUGAAAUGUGAGGUU | 597 | 780 |
| | #45 | | AUCUGUUUUU | | UAUAACACUCACAAGAAUCCUGAAAAGGAUGC | | |
| | | | | | CAAACGAAUACUGGCAUCUGUUUUU | | |
| 133 | exon | AAT | CUGGCAUCUG | 232 | ACAGCUUAUUUGGAAGCUGAAAUGUGAGGUU | 598 | 781 |
| | #45 | A | UUUUUGAGG | | UAUAACACUCACAAGAAUCCUGAAAAGGAUGC | | |
| | | | A | | CAAACCUGGCAUCUGUUUUUGAGGA | | |
| 134 | exon | ACT | GCAUCUGUUU | 233 | ACAGCUUAUUUGGAAGCUGAAAUGUGAGGUU | 599 | 782 |
| | #45 | G | UUGAGGAUU | | UAUAACACUCACAAGAAUCCUGAAAAGGAUGC | | |
| | | | G | | CAAACGCAUCUGUUUUUGAGGAUUG | | |
| 135 | exon | TCTG | UUUUUGAGG | 234 | ACAGCUUAUUUGGAAGCUGAAAUGUGAGGUU | 600 | 783 |
| | #45 | | AUUGCUGAAU | | UAUAACACUCACAAGAAUCCUGAAAAGGAUGC | | |
| | | | U | | CAAACUUUUUGAGGAUUGCUGAAUU | | |
| 136 | exon | AAT | AUUCAGCAAU | 235 | ACAGCUUAUUUGGAAGCUGAAAUGUGAGGUU | 601 | 784 |
| | #45 | A | CCUCAAAAAC | | UAUAACACUCACAAGAAUCCUGAAAAGGAUGC | | |
| | | | | | CAAACAUUCAGCAAUCCUCAAAAAC | | |
| 137 | exon | TTTG | AGGAUUGCUG | 236 | ACAGCUUAUUUGGAAGCUGAAAUGUGAGGUU | 602 | 785 |
| | #45 | | AAUUAUUUCU | | UAUAACACUCACAAGAAUCCUGAAAAGGAUGC | | |
| | | | | | CAAACAGGAUUGCUGAAUUAUUUCU | | |
| 138 | exon | ATTG | CUGAAUUAUU | 237 | ACAGCUUAUUUGGAAGCUGAAAUGUGAGGUU | 603 | 786 |
| | #45 | | UCUUCCCCAG | | UAUAACACUCACAAGAAUCCUGAAAAGGAUGC | | |
| | | | | | CAAACCUGAAUUAUUUCUUCCCCAG | | |
| 139 | exon | ACT | GGGAAGAAA | 238 | ACAGCUUAUUUGGAAGCUGAAAUGUGAGGUU | 604 | 787 |
| | #45 | G | UAAUUCAGCA | | UAUAACACUCACAAGAAUCCUGAAAAGGAUGC | | |
| | | | A | | CAAACGGGAAGAAAUAAUUCAGCAA | | |
| 140 | exon | GCT | AAUUAUUUCU | 239 | ACAGCUUAUUUGGAAGCUGAAAUGUGAGGUU | 605 | 788 |
| | #45 | G | UCCCCAGUUG | | UAUAACACUCACAAGAAUCCUGAAAAGGAUGC | | |
| | | | | | CAAACAAUUAUUUCUUCCCCAGUUG | | |
| 141 | exon | AAT | CAACUGGGGA | 240 | ACAGCUUAUUUGGAAGCUGAAAUGUGAGGUU | 606 | 789 |
| | #45 | G | AGAAAUAAU | | UAUAACACUCACAAGAAUCCUGAAAAGGAUGC | | |
| | | | U | | CAAACCAACUGGGGAAGAAAUAAUU | | |
| 142 | exon | ATTA | UUUCUUCCCC | 241 | ACAGCUUAUUUGGAAGCUGAAAUGUGAGGUU | 607 | 790 |
| | #45 | | AGUUGCAUUC | | UAUAACACUCACAAGAAUCCUGAAAAGGAUGC | | |
| | | | | | CAAACUUUCUUCCCCAGUUGCAUUC | | |
| 143 | exon | ATTG | AAUGCAACUG | 242 | ACAGCUUAUUUGGAAGCUGAAAUGUGAGGUU | 608 | 791 |
| | #45 | | GGGAAGAAA | | UAUAACACUCACAAGAAUCCUGAAAAGGAUGC | | |
| | | | U | | CAAACAAUGCAACUGGGGAAGAAAU | | |
| 144 | exon | GTTG | UCAGAACAUU | 243 | ACAGCUUAUUUGGAAGCUGAAAUGUGAGGUU | 609 | 792 |
| | #45 | | GAAUGCAACU | | UAUAACACUCACAAGAAUCCUGAAAAGGAUGC | | |
| | | | | | CAAACUCAGAACAUUGAAUGCAACU | | |
| 145 | exon | GTTG | CAUUCAAUGU | 244 | ACAGCUUAUUUGGAAGCUGAAAUGUGAGGUU | 610 | 793 |
| | #45 | | UCUGACAACA | | UAUAACACUCACAAGAAUCCUGAAAAGGAUGC | | |
| | | | | | CAAACCAUUCAAUGUUCUGACAACA | | |
| 146 | exon | ACT | UUGUCAGAAC | 245 | ACAGCUUAUUUGGAAGCUGAAAUGUGAGGUU | 611 | 794 |
| | #45 | G | AUUGAAUGCA | | UAUAACACUCACAAGAAUCCUGAAAAGGAUGC | | |
| | | | | | CAAACUUGUCAGAACAUUGAAUGCA | | |
| 147 | exon | AAT | UUCUGACAAC | 246 | ACAGCUUAUUUGGAAGCUGAAAUGUGAGGUU | 612 | 795 |
| | #45 | G | AGUUUGCCGC | | UAUAACACUCACAAGAAUCCUGAAAAGGAUGC | | |
| | | | | | CAAACUUCUGACAACAGUUUGCCGC | | |
| 148 | exon | TCTG | ACAACAGUUU | 247 | ACAGCUUAUUUGGAAGCUGAAAUGUGAGGUU | 613 | 796 |
| | #45 | | GCCGCUGCCC | | UAUAACACUCACAAGAAUCCUGAAAAGGAUGC | | |
| | | | | | CAAACACAACAGUUUGCCGCUGCCC | | |
| 149 | exon | ATTG | GGCAGCGGCA | 248 | ACAGCUUAUUUGGAAGCUGAAAUGUGAGGUU | 614 | 797 |
| | #45 | | AACUGUUGUC | | UAUAACACUCACAAGAAUCCUGAAAAGGAUGC | | |
| | | | | | CAAACGGCAGCGGCAAACUGUUGUC | | |
| 150 | exon | GAT | GCAUUGGGCA | 249 | ACAGCUUAUUUGGAAGCUGAAAUGUGAGGUU | 615 | 798 |
| | #45 | G | GCGGCAAACU | | UAUAACACUCACAAGAAUCCUGAAAAGGAUGC | | |
| | | | | | CAAACGCAUUGGGCAGCGGCAAACU | | |
| 151 | exon | TTTG | CCGCUGCCCA | 250 | ACAGCUUAUUUGGAAGCUGAAAUGUGAGGUU | 616 | 799 |
| | #45 | | AUGCCAUCCU | | UAUAACACUCACAAGAAUCCUGAAAAGGAUGC | | |
| | | | | | CAAACCCGCUGCCCAAUGCCAUCCU | | |
| 152 | exon | GCT | CCCAAUGCCA | 251 | ACAGCUUAUUUGGAAGCUGAAAUGUGAGGUU | 617 | 800 |
| | #45 | G | UCCUGGAGUU | | UAUAACACUCACAAGAAUCCUGAAAAGGAUGC | | |
| | | | | | CAAACCCCAAUGCCAUCCUGGAGUU | | |
| 153 | exon | CTTA | AGAUACCAUU | 252 | ACAGCUUAUUUGGAAGCUGAAAUGUGAGGUU | 618 | 801 |
| | #44 | | UGUAUUUAGC | | UAUAACACUCACAAGAAUCCUGAAAAGGAUGC | | |
| | | | | | CAAACAGAUACCAUUUGUAUUUAGC | | |
| 154 | exon | GCT | AAUACAAAUG | 253 | ACAGCUUAUUUGGAAGCUGAAAUGUGAGGUU | 619 | 802 |
| | #44 | A | GUAUCUUAAG | | UAUAACACUCACAAGAAUCCUGAAAAGGAUGC | | |
| | | | | | CAAACAAUACAAAUGGUAUCUUAAG | | |
| 155 | exon | CAT | CUAAAUACAA | 254 | ACAGCUUAUUUGGAAGCUGAAAUGUGAGGUU | 620 | 803 |
| | #44 | G | AUGGUAUCUU | | UAUAACACUCACAAGAAUCCUGAAAAGGAUGC | | |
| | | | | | CAAACCUAAAUACAAAUGGUAUCUU | | |
| 156 | exon | GAT | CCAUUUGUAU | 255 | ACAGCUUAUUUGGAAGCUGAAAUGUGAGGUU | 621 | 80 |
| | #44 | A | UUAGCAUGUU | | UAUAACACUCACAAGAAUCCUGAAAAGGAUGC | | |
| | | | | | CAAACCCAUUUGUAUUUAGCAUGUU | | |
| 157 | exon | ATTG | GGAACAUGCU | 256 | ACAGCUUAUUUGGAAGCUGAAAUGUGAGGUU | 622 | 805 |
| | #44 | | AAAUACAAAU | | UAUAACACUCACAAGAAUCCUGAAAAGGAUGC | | |
| | | | | | CAAACGGAACAUGCUAAAUACAAAU | | |
| 158 | exon | TTTG | UAUUUAGCAU | 257 | ACAGCUUAUUUGGAAGCUGAAAUGUGAGGUU | 623 | 806 |
| | #44 | | GUUCCCAAUU | | UAUAACACUCACAAGAAUCCUGAAAAGGAUGC | | |
| | | | | | CAAACUAUUUAGCAUGUUCCCAAUU | | |
| 159 | exon | TGTA | UUUAGCAUGU | 258 | ACAGCUUAUUUGGAAGCUGAAAUGUGAGGUU | 624 | 807 |
| | #44 | | UCCCAAUUCU | | UAUAACACUCACAAGAAUCCUGAAAAGGAUGC | | |
| | | | | | CAAACUUUAGCAUGUUCCCAAUUCU | | |
| 160 | exon | TTTA | GCAUGUUCCC | 259 | ACAGCUUAUUUGGAAGCUGAAAUGUGAGGUU | 625 | 808 |
| | #44 | | AAUUCUCAGG | | UAUAACACUCACAAGAAUCCUGAAAAGGAUGC | | |
| | | | | | CAAACGCAUGUUCCCAAUUCUCAGG | | |
| 161 | exon | CCTG | AGAAUUGGG | 260 | ACAGCUUAUUUGGAAGCUGAAAUGUGAGGUU | 626 | 809 |
| | #44 | | AACAUGCUAA | | UAUAACACUCACAAGAAUCCUGAAAAGGAUGC | | |
| | | | A | | CAAACAGAAUUGGGAACAUGCUAAA | | |
| 162 | exon | CAT | UUCCCAAUUC | 261 | ACAGCUUAUUUGGAAGCUGAAAUGUGAGGUU | 627 | 810 |
| | #44 | G | UCAGGAAUUU | | UAUAACACUCACAAGAAUCCUGAAAAGGAUGC | | |
| | | | | | CAAACUUCCCAAUUCUCAGGAAUUU | | |
| 163 | exon | TTTG | UGUCUUUCUG | 262 | ACAGCUUAUUUGGAAGCUGAAAUGUGAGGUU | 628 | 811 |
| | #44 | | AGAAACUGUU | | UAUAACACUCACAAGAAUCCUGAAAAGGAUGC | | |
| | | | | | CAAACUGUCUUUCUGAGAAACUGUU | | |
| 164 | exon | TGTG | UCUUUCUGAG | 263 | ACAGCUUAUUUGGAAGCUGAAAUGUGAGGUU | 629 | 812 |
| | #44 | | AAACUGUUCA | | UAUAACACUCACAAGAAUCCUGAAAAGGAUGC | | |
| | | | | | CAAACUCUUUCUGAGAAACUGUUCA | | |
| 165 | exon | GCT | AACAGUUUCU | 264 | ACAGCUUAUUUGGAAGCUGAAAUGUGAGGUU | 630 | 813 |
| | #44 | G | CAGAAAGACA | | UAUAACACUCACAAGAAUCCUGAAAAGGAUGC | | |
| | | | | | CAAACAACAGUUUCUCAGAAAGACA | | |
| 166 | exon | TCTG | AGAAACUGUU | 265 | ACAGCUUAUUUGGAAGCUGAAAUGUGAGGUU | 631 | 814 |
| | #44 | | CAGCUUCUGU | | UAUAACACUCACAAGAAUCCUGAAAAGGAUGC | | |
| | | | | | CAAACAGAAACUGUUCAGCUUCUGU | | |
| 167 | exon | GCT | ACAGAAGCUG | 266 | ACAGCUUAUUUGGAAGCUGAAAUGUGAGGUU | 632 | 815 |
| | #44 | A | AACAGUUUCU | | UAUAACACUCACAAGAAUCCUGAAAAGGAUGC | | |
| | | | | | CAAACACAGAAGCUGAACAGUUUCU | | |
| 168 | exon | ACT | UUCAGCUUCU | 267 | ACAGCUUAUUUGGAAGCUGAAAUGUGAGGUU | 633 | 816 |
| | #44 | G | GUUAGCCACU | | UAUAACACUCACAAGAAUCCUGAAAAGGAUGC | | |
| | | | | | CAAACUUCAGCUUCUGUUAGCCACU | | |
| 169 | exon | AGT | GCUAACAGAA | 268 | ACAGCUUAUUUGGAAGCUGAAAUGUGAGGUU | 634 | 817 |
| | #44 | G | GCUGAACAGU | | UAUAACACUCACAAGAAUCCUGAAAAGGAUGC | | |
| | | | | | CAAACGCUAACAGAAGCUGAACAGU | | |
| 170 | exon | TTTA | AUCAGUGGCU | 269 | ACAGCUUAUUUGGAAGCUGAAAUGUGAGGUU | 635 | 818 |
| | #44 | | AACAGAAGCU | | UAUAACACUCACAAGAAUCCUGAAAAGGAUGC | | |
| | | | | | CAAACAUCAGUGGCUAACAGAAGCU | | |
| 171 | exon | TCTG | UUAGCCACUG | 270 | ACAGCUUAUUUGGAAGCUGAAAUGUGAGGUU | 636 | 819 |
| | #44 | | AUUAAAUAUC | | UAUAACACUCACAAGAAUCCUGAAAAGGAUGC | | |
| | | | | | CAAACUUAGCCACUGAUUAAAUAUC | | |
| 172 | exon | GAT | UUUAAUCAGU | 271 | ACAGCUUAUUUGGAAGCUGAAAUGUGAGGUU | 637 | 820 |
| | #44 | A | GGCUAACAGA | | UAUAACACUCACAAGAAUCCUGAAAAGGAUGC | | |
| | | | | | CAAACUUUAAUCAGUGGCUAACAGA | | |
| 173 | exon | GTTA | GCCACUGAUU | 272 | ACAGCUUAUUUGGAAGCUGAAAUGUGAGGUU | 638 | 821 |
| | #44 | | AAAUAUCUUU | | UAUAACACUCACAAGAAUCCUGAAAAGGAUGC | | |
| | | | | | CAAACGCCACUGAUUAAAUAUCUUU | | |
| 174 | exon | TATA | AAGAUAUUU | 273 | ACAGCUUAUUUGGAAGCUGAAAUGUGAGGUU | 639 | 822 |
| | #44 | | AAUCAGUGGC | | UAUAACACUCACAAGAAUCCUGAAAAGGAUGC | | |
| | | | U | | CAAACAAGAUAUUUAAUCAGUGGCU | | |
| 175 | exon | GAT | UAAAGAUAU | 274 | ACAGCUUAUUUGGAAGCUGAAAUGUGAGGUU | 640 | 823 |
| | #44 | A | UUAAUCAGUG | | UAUAACACUCACAAGAAUCCUGAAAAGGAUGC | | |
| | | | G | | CAAACUAAAGAUAUUUAAUCAGUGG | | |
| 176 | exon | TTTA | UAUCAUAAUG | 275 | ACAGCUUAUUUGGAAGCUGAAAUGUGAGGUU | 641 | 824 |
| | #44 | | AAAACGCCGC | | UAUAACACUCACAAGAAUCCUGAAAAGGAUGC | | |
| | | | | | CAAACUAUCAUAAUGAAAACGCCGC | | |
| 177 | exon | TATA | UCAUAAUGAA | 276 | ACAGCUUAUUUGGAAGCUGAAAUGUGAGGUU | 642 | 825 |
| | #44 | | AACGCCGCCA | | UAUAACACUCACAAGAAUCCUGAAAAGGAUGC | | |
| | | | | | CAAACUCAUAAUGAAAACGCCGCCA | | |
| 178 | exon | AAT | GCGGCGUUUU | 277 | ACAGCUUAUUUGGAAGCUGAAAUGUGAGGUU | 643 | 826 |
| | #44 | G | CAUUAUGAUA | | UAUAACACUCACAAGAAUCCUGAAAAGGAUGC | | |
| | | | | | CAAACGCGGCGUUUUCAUUAUGAUA | | |
| 179 | exon | CAT | AUGAAAACGC | 278 | ACAGCUUAUUUGGAAGCUGAAAUGUGAGGUU | 644 | 827 |
| | #44 | A | CGCCAUUUCU | | UAUAACACUCACAAGAAUCCUGAAAAGGAUGC | | |
| | | | | | CAAACAUGAAAACGCCGCCAUUUCU | | |
| 180 | exon | AAT | AAAACGCCGC | 279 | ACAGCUUAUUUGGAAGCUGAAAUGUGAGGUU | 645 | 828 |
| | #44 | G | CAUUUCUCAA | | UAUAACACUCACAAGAAUCCUGAAAAGGAUGC | | |
| | | | | | CAAACAAAACGCCGCCAUUUCUCAA | | |
| 181 | exon | GTTG | AGAAAUGGCG | 280 | ACAGCUUAUUUGGAAGCUGAAAUGUGAGGUU | 646 | 829 |
| | #44 | | GCGUUUUCAU | | UAUAACACUCACAAGAAUCCUGAAAAGGAUGC | | |
| | | | | | CAAACAGAAAUGGCGGCGUUUUCAU | | |
| 182 | exon | TCTG | UUGAGAAAU | 281 | ACAGCUUAUUUGGAAGCUGAAAUGUGAGGUU | 647 | 830 |
| | #44 | | GGCGGCGUUU | | UAUAACACUCACAAGAAUCCUGAAAAGGAUGC | | |
| | | | U | | CAAACUUGAGAAAUGGCGGCGUUUU | | |
| 183 | exon | TTTG | ACAGAUCUGU | 282 | ACAGCUUAUUUGGAAGCUGAAAUGUGAGGUU | 648 | 831 |
| | #44 | | UGAGAAAUG | | UAUAACACUCACAAGAAUCCUGAAAAGGAUGC | | |
| | | | G | | CAAACACAGAUCUGUUGAGAAAUGG | | |

TABLE 7 provide exemplary retRNA sequences designed to pair with CasM.265466 and the corresponding gRNA sequences in TABLE 6 with the same targeting site numbering. The numbering of the targeting sites in TABLE 7 aligns with the numbering in TABLE 6.

TABLE 7
Exemplary CasM.265466 retRNA sequences

| | | SEQ | | SEQ | retRNA | SEQ | | SEQ |
|---|---|---|---|---|---|---|---|---|
| Target | | ID | | ID | se- | ID | | ID |
| Site | PBS | NO: | RTT | NO: | quence | NO: | retRNA sequence with scaffold region | NO: |
| 31 | UU | 832 | UUG | 856 | UUGAA | 880 | GUGCUCGCUUCGGCAGCACAUAUACUAGUCGACGGGCCGCAC | 904 |
| | AA | | AAU | | UGAAG | | UCGCCGGUCCCAAGCCCGGAUAAAAUGGGAGGGGGCGGGAA | |
| | AG | | GAA | | UACAU | | ACCGCCUAACCAUGCCGAGUGCGGCCGCAACUAAGCACAUGA | |
| | GA | | GUA | | GUUAA | | GGAUCACCCAUGUGCUUGAAUGAAGUACAUGUUAAAGGAUU | |
| | UU | | CAU | | AGGAU | | CAAAUUAACAGUGGCCGCGGUCGGCGUGGACUGUAGAACAC | |
| | C | | G | | UC | | UGCCAAUGCCGGUCCCAAGCCCGGAUAAAAGUGGAGGGUAC | |
| | | | | | | | AGUCCACGCUCUAGAGCGGACUUCGGUCCGC | |
| 35 | UU | 833 | CGG | 857 | CGGAG | 881 | GUGCUCGCUUCGGCAGCACAUAUACUAGUCGACGGGCCGCAC | 905 |
| | GA | | AGG | | GCAAG | | UCGCCGGUCCCAAGCCCGGAUAAAAUGGGAGGGGGCGGGAA | |
| | AU | | CAA | | UACCA | | ACCGCCUAACCAUGCCGAGUGCGGCCGCAACUAAGCACAUGA | |
| | GA | | GUA | | GUUGA | | GGAUCACCCAUGUGCCGGAGGCAAGUACCAGUUGAAUGAAA | |
| | AA | | CCA | | AUGAA | | UAAAUUAACAGUGGCCGCGGUCGGCGUGGACUGUAGAACAC | |
| | U | | G | | AU | | UGCCAAUGCCGGUCCCAAGCCCGGAUAAAAGUGGAGGGUAC | |
| | | | | | | | AGUCCACGCUCUAGAGCGGACUUCGGUCCGC | |
| 40 | UG | 834 | CGG | 858 | CGGUU | 882 | GUGCUCGCUUCGGCAGCACAUAUACUAGUCGACGGGCCGCAC | 906 |
| | UU | | UUC | | CUGAG | | UCGCCGGUCCCAAGCCCGGAUAAAAUGGGAGGGGGCGGGAA | |
| | CU | | UGA | | UACAG | | ACCGCCUAACCAUGCCGAGUGCGGCCGCAACUAAGCACAUGA | |
| | UG | | GUA | | GUGUU | | GGAUCACCCAUGUGCCGGUUCUGAGUACAGGUGUUCUUGUA | |
| | UA | | CAG | | CUUGU | | CAAAUUAACAGUGGCCGCGGUCGGCGUGGACUGUAGAACAC | |
| | C | | G | | AC | | UGCCAAUGCCGGUCCCAAGCCCGGAUAAAAGUGGAGGGUAC | |
| | | | | | | | AGUCCACGCUCUAGAGCGGACUUCGGUCCGC | |
| 41 | UG | 835 | GAA | 859 | GAAUC | 883 | GUGCUCGCUUCGGCAGCACAUAUACUAGUCGACGGGCCGCAC | 907 |
| | AA | | UCA | | AGUGG | | UCGCCGGUCCCAAGCCCGGAUAAAAUGGGAGGGGGCGGGAA | |
| | GU | | GUG | | UACGG | | ACCGCCUAACCAUGCCGAGUGCGGCCGCAACUAAGCACAUGA | |
| | AC | | GUA | | AUGAA | | GGAUCACCCAUGUGCGAAUCAGUGGUACGGAUGAAGUACAA | |
| | AA | | CGG | | GUACA | | GAAAUUAACAGUGGCCGCGGUCGGCGUGGACUGUAGAACAC | |
| | G | | A | | AG | | UGCCAAUGCCGGUCCCAAGCCCGGAUAAAAGUGGAGGGUAC | |
| | | | | | | | AGUCCACGCUCUAGAGCGGACUUCGGUCCGC | |
| 42 | UU | 836 | CUG | 860 | CUGAA | 884 | GUGCUCGCUUCGGCAGCACAUAUACUAGUCGACGGGCCGCAC | 908 |
| | GU | | AAG | | GGUGG | | UCGCCGGUCCCAAGCCCGGAUAAAAUGGGAGGGGGCGGGAA | |
| | AC | | GUG | | UACUU | | ACCGCCUAACCAUGCCGAGUGCGGCCGCAACUAAGCACAUGA | |
| | UU | | GUA | | CUUGU | | GGAUCACCCAUGUGCCUGAAGGUGGUACUUCUUGUACUUCA | |
| | CA | | CUU | | ACUUC | | UAAAUUAACAGUGGCCGCGGUCGGCGUGGACUGUAGAACAC | |
| | U | | C | | AU | | UGCCAAUGCCGGUCCCAAGCCCGGAUAAAAGUGGAGGGUAC | |
| | | | | | | | AGUCCACGCUCUAGAGCGGACUUCGGUCCGC | |
| 43 | GU | 837 | AAU | 861 | AAUUC | 885 | GUGCUCGCUUCGGCAGCACAUAUACUAGUCGACGGGCCGCAC | 909 |
| | GG | | UCA | | AGAAG | | UCGCCGGUCCCAAGCCCGGAUAAAAUGGGAGGGGGCGGGAA | |
| | GA | | GAA | | UACUC | | ACCGCCUAACCAUGCCGAGUGCGGCCGCAACUAAGCACAUGA | |
| | UG | | GUA | | AGUGG | | GGAUCACCCAUGUGCAAUUCAGAAGUACUCAGUGGGAUGAA | |
| | AA | | CUC | | GAUGA | | GAAAUUAACAGUGGCCGCGGUCGGCGUGGACUGUAGAACAC | |
| | G | | A | | AG | | UGCCAAUGCCGGUCCCAAGCCCGGAUAAAAGUGGAGGGUAC | |
| | | | | | | | AGUCCACGCUCUAGAGCGGACUUCGGUCCGC | |
| 44 | CA | 838 | AGA | 862 | AGAAU | 886 | GUGCUCGCUUCGGCAGCACAUAUACUAGUCGACGGGCCGCAC | 910 |
| | GU | | AUU | | UCAGG | | UCGCCGGUCCCAAGCCCGGAUAAAAUGGGAGGGGGGGGAA | |
| | GG | | CAG | | UACAA | | ACCGCCUAACCAUGCCGAGUGCGGCCGCAACUAAGCACAUGA | |
| | GA | | GUA | | UCAGU | | GGAUCACCCAUGUGCAGAAUUCAGGUACAAUCAGUGGGAUG | |
| | UG | | CAA | | GGGAU | | AAAAUUAACAGUGGCCGCGGUCGGCGUGGACUGUAGAACAC | |
| | A | | U | | GA | | UGCCAAUGCCGGUCCCAAGCCCGGAUAAAAGUGGAGGGUAC | |
| | | | | | | | AGUCCACGCUCUAGAGCGGACUUCGGUCCGC | |
| 49 | GC | 839 | AAA | 863 | AAAAC | 887 | GUGCUCGCUUCGGCAGCACAUAUACUAGUCGACGGGCCGCAC | 911 |
| | AA | | ACA | | AAGAG | | UCGCCGGUCCCAAGCCCGGAUAAAAUGGGAGGGGGCGGGAA | |
| | UC | | AGA | | GUACC | | ACCGCCUAACCAUGCCGAGUGCGGCCGCAACUAAGCACAUGA | |
| | AA | | GGU | | CAGCA | | GGAUCACCCAUGUGCAAAACAAGAGGUACCCAGCAAUCAAG | |
| | GA | | ACC | | AUCAA | | AGAAAUUAACAGUGGCCGCGGUCGGCGUGGACUGUAGAACA | |
| | G | | CA | | GAG | | CUGCCAAUGCCGGUCCCAAGCCCGGAUAAAAGUGGAGGGUAC | |
| | | | | | | | AGUCCACGCUCUAGAGCGGACUUCGGUCCGC | |
| 51 | UG | 840 | CCU | 864 | CCUCU | 888 | GUGCUCGCUUCGGCAGCACAUAUACUAGUCGACGGGCCGCAC | 912 |
| | GU | | CUU | | UGAUG | | UCGCCGGUCCCAAGCCCGGAUAAAAUGGGAGGGGGCGGGAA | |
| | CU | | GAU | | GUACU | | ACCGCCUAACCAUGCCGAGUGCGGCCGCAACUAAGCACAUGA | |
| | UG | | GGU | | GCUGG | | GGAUCACCCAUGUGCCCUCUUGAUGGUACUGCUGGUCUUGU | |
| | UU | | ACU | | UCUUG | | UUAAAUUAACAGUGGCCGCGGUCGGCGUGGACUGUAGAACA | |
| | U | | GC | | UUU | | CUGCCAAUGCCGGUCCCAAGCCCGGAUAAAAGUGGAGGGUAC | |
| | | | | | | | AGUCCACGCUCUAGAGCGGACUUCGGUCCGC | |
| 57 | CA | 841 | GUU | 865 | GUUGG | 889 | GUGCUCGCUUCGGCAGCACAUAUACUAGUCGACGGGCCGCAC | 913 |
| | UU | | GGA | | AAGAG | | UCGCCGGUCCCAAGCCCGGAUAAAAUGGGAGGGGGCGGGAA | |
| | AC | | AGA | | GUACA | | ACCGCCUAACCAUGCCGAGUGCGGCCGCAACUAAGCACAUGA | |
| | CG | | GGU | | CUCAU | | GGAUCACCCAUGUGCGUUGGAAGAGGUACACUCAUUACCGC | |
| | CU | | ACA | | UACCG | | UGAAAUUAACAGUGGCCGCGGUCGGCGUGGACUGUAGAACA | |
| | G | | CU | | CUG | | CUGCCAAUGCCGGUCCCAAGCCCGGAUAAAAGUGGAGGGUAC | |
| | | | | | | | AGUCCACGCUCUAGAGCGGACUUCGGUCCGC | |
| 58 | UA | 842 | UUU | 866 | UUUGG | 890 | GUGCUCGCUUCGGCAGCACAUAUACUAGUCGACGGGCCGCAC | 914 |
| | AU | | GGG | | GCAGG | | UCGCCGGUCCCAAGCCCGGAUAAAAUGGGAGGGGGCGGGAA | |
| | GA | | CAG | | GUACC | | ACCGCCUAACCAUGCCGAGUGCGGCCGCAACUAAGCACAUGA | |
| | GU | | GGU | | GGUAA | | GGAUCACCCAUGUGCUUUGGGCAGGGUACCGGUAAUGAGUU | |
| | UC | | ACC | | UGAGU | | CUAAAUUAACAGUGGCCGCGGUCGGCGUGGACUGUAGAACA | |
| | U | | GG | | UCU | | CUGCCAAUGCCGGUCCCAAGCCCGGAUAAAAGUGGAGGGUAC | |
| | | | | | | | AGUCCACGCUCUAGAGCGGACUUCGGUCCGC | |
| 59 | UG | 843 | GGC | 867 | GGCGU | 891 | GUGCUCGCUUCGGCAGCACAUAUACUAGUCGACGGGCCGCAC | 915 |
| | GA | | GUC | | CCCCG | | UCGCCGGUCCCAAGCCCGGAUAAAAUGGGAGGGGGCGGGAA | |
| | AG | | CCC | | GUACA | | ACCGCCUAACCAUGCCGAGUGCGGCCGCAACUAAGCACAUGA | |
| | AA | | GGU | | GUUGG | | GGAUCACCCAUGUGCGGCGUCCCCGGUACAGUUGGAAGAACU | |
| | CU | | ACA | | AAGAA | | CAAAUUAACAGUGGCCGCGGUCGGCGUGGACUGUAGAACAC | |
| | C | | GU | | CUC | | UGCCAAUGCCGGUCCCAAGCCCGGAUAAAAGUGGAGGGUAC | |
| | | | | | | | AGUCCACGCUCUAGAGCGGACUUCGGUCCGC | |
| 60 | AG | 844 | AGA | 868 | AGAGG | 892 | GUGCUCGCUUCGGCAGCACAUAUACUAGUCGACGGGCCGCAC | 916 |
| | UU | | GGC | | CGUCG | | UCGCCGGUCCCAAGCCCGGAUAAAAUGGGAGGGGGCGGGAA | |
| | GG | | GUC | | GUACC | | ACCGCCUAACCAUGCCGAGUGCGGCCGCAACUAAGCACAUGA | |
| | AA | | GGU | | CCAGU | | GGAUCACCCAUGUGCAGAGGCGUCGGUACCCCAGUUGGAAG | |
| | GA | | ACC | | UGGAA | | AAAAAUUAACAGUGGCCGCGGUCGGCGUGGACUGUAGAACA | |
| | A | | CC | | GAA | | CUGCCAAUGCCGGUCCCAAGCCCGGAUAAAAGUGGAGGGUAC | |
| | | | | | | | AGUCCACGCUCUAGAGCGGACUUCGGUCCGC | |
| 61 | GA | 845 | CUU | 869 | CUUCC | 893 | GUGCUCGCUUCGGCAGCACAUAUACUAGUCGACGGGCCGCAC | 917 |
| | CG | | CCA | | AACUG | | UCGCCGGUCCCAAGCCCGGAUAAAAUGGGAGGGGGCGGGAA | |
| | CC | | ACU | | GUACG | | ACCGCCUAACCAUGCCGAGUGCGGCCGCAACUAAGCACAUGA | |
| | UC | | GGU | | GGGAC | | GGAUCACCCAUGUGCCUUCCAACUGGUACGGGGACGCCUCUG | |
| | UG | | ACG | | GCCUC | | UAAAUUAACAGUGGCCGCGGUCGGCGUGGACUGUAGAACAC | |
| | U | | GG | | UGU | | UGCCAAUGCCGGUCCCAAGCCCGGAUAAAAGUGGAGGGUAC | |
| | | | | | | | AGUCCACGCUCUAGAGCGGACUUCGGUCCGC | |
| 62 | AC | 846 | GCA | 870 | GCAGG | 894 | GUGCUCGCUUCGGCAGCACAUAUACUAGUCGACGGGCCGCAC | 918 |
| | AG | | GGA | | AUUUG | | UCGCCGGUCCCAAGCCCGGAUAAAAUGGGAGGGGGCGGGAA | |
| | AG | | UUU | | GUACG | | ACCGCCUAACCAUGCCGAGUGCGGCCGCAACUAAGCACAUGA | |
| | GC | | GGU | | GAACA | | GGAUCACCCAUGUGCGCAGGAUUUGGUACGGAACAGAGGCG | |
| | GU | | ACG | | GAGGC | | UCAAAUUAACAGUGGCCGCGGUCGGCGUGGACUGUAGAACA | |
| | C | | GA | | GUC | | CUGCCAAUGCCGGUCCCAAGCCCGGAUAAAAGUGGAGGGUAC | |
| | | | | | | | AGUCCACGCUCUAGAGCGGACUUCGGUCCGC | |
| 63 | GU | 847 | GGG | 871 | GGGGA | 895 | GUGCUCGCUUCGGCAGCACAUAUACUAGUCGACGGGCCGCAC | 919 |
| | UC | | GAC | | CGCCG | | UCGCCGGUCCCAAGCCCGGAUAAAAUGGGAGGGGGCGGGAA | |
| | CA | | GCC | | GUACU | | ACCGCCUAACCAUGCCGAGUGCGGCCGCAACUAAGCACAUGA | |
| | AA | | GGU | | CUGUU | | GGAUCACCCAUGUGCGGGGACGCCGGUACUCUGUUCCAAAUC | |
| | UC | | ACU | | CCAAA | | CAAAUUAACAGUGGCCGCGGUCGGCGUGGACUGUAGAACAC | |
| | C | | CU | | UCC | | UGCCAAUGCCGGUCCCAAGCCCGGAUAAAAGUGGAGGGUAC | |
| | | | | | | | AGUCCACGCUCUAGAGCGGACUUCGGUCCGC | |
| 64 | GC | 848 | ACA | 872 | ACAGG | 896 | GUGCUCGCUUCGGCAGCACAUAUACUAGUCGACGGGCCGCAC | 920 |
| | AG | | GGC | | CAACG | | UCGCCGGUCCCAAGCCCGGAUAAAAUGGGAGGGGGCGGGAA | |
| | GA | | AAC | | GUACA | | ACCGCCUAACCAUGCCGAGUGCGGCCGCAACUAAGCACAUGA | |
| | UU | | GGU | | AUGCA | | GGAUCACCCAUGUGCACAGGCAACGGUACAAUGCAGGAUUU | |
| | UG | | ACA | | GGAUU | | GGAAAUUAACAGUGGCCGCGGUCGGCGUGGACUGUAGAACA | |
| | G | | AU | | UGG | | CUGCCAAUGCCGGUCCCAAGCCCGGAUAAAAGUGGAGGGUAC | |
| | | | | | | | AGUCCACGCUCUAGAGCGGACUUCGGUCCGC | |
| 115 | UA | 849 | UAC | 873 | UACUC | 897 | GUGCUCGCUUCGGCAGCACAUAUACUAGUCGACGGGCCGCAC | 921 |
| | CU | | UCA | | AGACG | | UCGCCGGUCCCAAGCCCGGAUAAAAUGGGAGGGGGCGGGAA | |
| | CU | | GAC | | UACUG | | ACCGCCUAACCAUGCCGAGUGCGGCCGCAACUAAGCACAUGA | |
| | GG | | GUA | | UUACU | | GGAUCACCCAUGUGCUACUCAGACGUACUGUUACUCUGGUG | |
| | UG | | CUG | | CUGGU | | AAAAUUAACAGUGGCCGCGGUCGGCGUGGACUGUAGAACAC | |
| | A | | U | | GA | | UGCCAAUGCCGGUCCCAAGCCCGGAUAAAAGUGGAGGGUAC | |
| | | | | | | | AGUCCACGCUCUAGAGCGGACUUCGGUCCGC | |
| 116 | GU | 850 | CCU | 874 | CCUAC | 898 | GUGCUCGCUUCGGCAGCACAUAUACUAGUCGACGGGCCGCAC | 922 |
| | UA | | ACU | | UCAGG | | UCGCCGGUCCCAAGCCCGGAUAAAAUGGGAGGGGGGGGAA | |
| | CU | | CAG | | UACAC | | ACCGCCUAACCAUGCCGAGUGCGGCCGCAACUAAGCACAUGA | |
| | CU | | GUA | | UGUUA | | GGAUCACCCAUGUGCCCUACUCAGGUACACUGUUACUCUGGU | |
| | GG | | CAC | | CUCUG | | AAAUUAACAGUGGCCGCGGUCGGCGUGGACUGUAGAACACU | |
| | U | | U | | GU | | GCCAAUGCCGGUCCCAAGCCCGGAUAAAAGUGGAGGGUACA | |
| | | | | | | | GUCCACGCUCUAGAGCGGACUUCGGUCCGC | |
| 117 | GU | 851 | UGU | 875 | UGUGU | 899 | GUGCUCGCUUCGGCAGCACAUAUACUAGUCGACGGGCCGCAC | 923 |
| | AA | | GUC | | CACCG | | UCGCCGGUCCCAAGCCCGGAUAAAAUGGGAGGGGGCGGGAA | |
| | CA | | ACC | | UACAG | | ACCGCCUAACCAUGCCGAGUGCGGCCGCAACUAAGCACAUGA | |
| | GU | | GUA | | AGUAA | | GGAUCACCCAUGUGCUGUGUCACCGUACAGAGUAACAGUCU | |
| | CU | | CAG | | CAGUC | | GAAAUUAACAGUGGCCGCGGUCGGCGUGGACUGUAGAACAC | |
| | G | | A | | UG | | UGCCAAUGCCGGUCCCAAGCCCGGAUAAAAGUGGAGGGUAC | |
| | | | | | | | AGUCCACGCUCUAGAGCGGACUUCGGUCCGC | |
| 137 | AU | 852 | GGG | 876 | GGGGA | 900 | GUGCUCGCUUCGGCAGCACAUAUACUAGUCGACGGGCCGCAC | 924 |
| | UC | | GAA | | AGAAG | | UCGCCGGUCCCAAGCCCGGAUAAAAUGGGAGGGGGCGGGAA | |
| | AG | | GAA | | UACAU | | ACCGCCUAACCAUGCCGAGUGCGGCCGCAACUAAGCACAUGA | |
| | CA | | GUA | | AAUUC | | GGAUCACCCAUGUGCGGGGAAGAAGUACAUAAUUCAGCAAU | |
| | AU | | CAU | | AGCAA | | CAAAUUAACAGUGGCCGCGGUCGGCGUGGACUGUAGAACAC | |
| | C | | A | | UC | | UGCCAAUGCCGGUCCCAAGCCCGGAUAAAAGUGGAGGGUAC | |
| | | | | | | | AGUCCACGCUCUAGAGCGGACUUCGGUCCGC | |
| 145 | AG | 853 | CAA | 877 | CAAAC | 901 | GUGCUCGCUUCGGCAGCACAUAUACUAGUCGACGGGCCGCAC | 925 |
| | AA | | ACU | | UGUUG | | UCGCCGGUCCCAAGCCCGGAUAAAAUGGGAGGGGGCGGGAA | |
| | CA | | GUU | | UACGU | | ACCGCCUAACCAUGCCGAGUGCGGCCGCAACUAAGCACAUGA | |
| | UU | | GUA | | CAGAA | | GGAUCACCCAUGUGCCAAACUGUUGUACGUCAGAACAUUGA | |
| | GA | | CGU | | CAUUG | | AAAAUUAACAGUGGCCGCGGUCGGCGUGGACUGUAGAACAC | |
| | A | | C | | AA | | UGCCAAUGCCGGUCCCAAGCCCGGAUAAAAGUGGAGGGUAC | |
| | | | | | | | AGUCCACGCUCUAGAGCGGACUUCGGUCCGC | |
| 150 | CG | 854 | ACA | 878 | ACAAC | 902 | GUGCUCGCUUCGGCAGCACAUAUACUAGUCGACGGGCCGCAC | 926 |
| | CU | | ACA | | AGUUG | | UCGCCGGUCCCAAGCCCGGAUAAAAUGGGAGGGGGCGGGAA | |
| | GC | | GUU | | UACUG | | ACCGCCUAACCAUGCCGAGUGCGGCCGCAACUAAGCACAUGA | |
| | CC | | GUA | | CCGCU | | GGAUCACCCAUGUGCACAACAGUUGUACUGCCGCUGCCCAAU | |
| | AA | | CUG | | GCCCA | | AAAUUAACAGUGGCCGCGGUCGGCGUGGACUGUAGAACACU | |
| | U | | C | | AU | | GCCAAUGCCGGUCCCAAGCCCGGAUAAAAGUGGAGGGUACA | |
| | | | | | | | GUCCACGCUCUAGAGCGGACUUCGGUCCGC | |
| 152 | GG | 855 | ACA | 879 | ACAGG | 903 | GUGCUCGCUUCGGCAGCACAUAUACUAGUCGACGGGCCGCAC | 927 |
| | AU | | GGA | | AACUG | | UCGCCGGUCCCAAGCCCGGAUAAAAUGGGAGGGGGCGGGAA | |
| | GG | | ACU | | UACCC | | ACCGCCUAACCAUGCCGAGUGCGGCCGCAACUAAGCACAUGA | |
| | CA | | GUA | | AGGAU | | GGAUCACCCAUGUGCACAGGAACUGUACCCAGGAUGGCAUU | |
| | UU | | CCC | | GGCAU | | GAAAUUAACAGUGGCCGCGGUCGGCGUGGACUGUAGAACAC | |
| | G | | A | | UG | | UGCCAAUGCCGGUCCCAAGCCCGGAUAAAAGUGGAGGGUAC | |
| | | | | | | | AGUCCACGCUCUAGAGCGGACUUCGGUCCGC | |

TABLE 8 provides exemplary gRNA Sequences designed to be used in combination with CasPhi.12 for targeting the DMD gene. The repeat sequence is the full guide sequence minus the spacer sequence. In general, the gRNAs described in TABLE 8 have a repeat sequence of SEQ ID NO: 6. An exemplary reference number for the target, DMD, is NCBI Accession Number, NM_004006.3. In some embodiments, the gRNA may be represented by the full gRNA sequence with a transcript initiating guanine (G).

TABLE 8
Exemplary CasPhi.12 gRNA Sequences

| | | | | | | | gRNA sequence |
|---|---|---|---|---|---|---|---|
| | Target | | | SEQ | | SEQ | (transcription |
| Target | (DMD | | Spacer | ID | Full gRNA sequence | ID | initiating G) |
| Site | exon) | PAM | Sequence | NO: | (Repeat + Spacer) | NO: | SEQ ID NO: |
| 1 | exon #53 | CTTG | GUUUCUGUGA | 928 | AUUGCUCCUUACGAGGAGACGUUU | 1212 | 1354 |
| | | | UUUUCUU | | CUGUGAUUUUCUU | | |
| 2 | exon #53 | GTTT | CUGUGAUUUU | 929 | AUUGCUCCUUACGAGGAGACCUGU | 1213 | 1355 |
| | | | CUUUUGG | | GAUUUUCUUUUGG | | |
| 3 | exon #53 | TTTC | UGUGAUUUUC | 930 | AUUGCUCCUUACGAGGAGACUGUG | 1214 | 1356 |
| | | | UUUUGGA | | AUUUUCUUUUGGA | | |
| 4 | exon #53 | ATTT | UCUUUUGGAU | 931 | AUUGCUCCUUACGAGGAGACUCUU | 1215 | 1357 |
| | | | UGCAUCU | | UUGGAUUGCAUCU | | |
| 5 | exon #53 | TTTT | CUUUUGGAUU | 932 | AUUGCUCCUUACGAGGAGACCUUU | 1216 | 1358 |
| | | | GCAUCUA | | UGGAUUGCAUCUA | | |
| 6 | exon #53 | TTTC | UUUUGGAUU | 933 | AUUGCUCCUUACGAGGAGACUUUU | 1217 | 1359 |
| | | | GCAUCUAC | | GGAUUGCAUCUAC | | |
| 7 | exon #53 | CTTT | UGGAUUGCAU | 934 | AUUGCUCCUUACGAGGAGACUGGA | 1218 | 1360 |
| | | | CUACUGU | | UUGCAUCUACUGU | | |
| 8 | exon #53 | TTTT | GGAUUGCAUC | 935 | AUUGCUCCUUACGAGGAGACGGAU | 1219 | 1361 |
| | | | UACUGUA | | UGCAUCUACUGUA | | |
| 9 | exon #53 | TTTG | GAUUGCAUCU | 936 | AUUGCUCCUUACGAGGAGACGAUU | 1220 | 1362 |
| | | | ACUGUAU | | GCAUCUACUGUAU | | |
| 10 | exon #53 | ATTG | CAUCUACUGU | 937 | AUUGCUCCUUACGAGGAGACCAUC | 1221 | 1363 |
| | | | AUAGGGA | | UACUGUAUAGGGA | | |
| 11 | exon #53 | CTTG | AGUCAUGGAA | 938 | AUUGCUCCUUACGAGGAGACAGUC | 1222 | 1364 |
| | | | GGAGGGU | | AUGGAAGGAGGGU | | |
| 12 | exon #53 | CTTC | CAUGACUCAA | 939 | AUUGCUCCUUACGAGGAGACCAUG | 1223 | 1365 |
| | | | GCUUGGC | | ACUCAAGCUUGGC | | |
| 13 | exon #53 | CTTG | GCUCUGGCCU | 940 | AUUGCUCCUUACGAGGAGACGCUC | 1224 | 1366 |
| | | | GUCCUAA | | UGGCCUGUCCUAA | | |
| 14 | exon #53 | CTTA | GGACAGGCCA | 941 | AUUGCUCCUUACGAGGAGACGGAC | 1225 | 1367 |
| | | | GAGCCAA | | AGGCCAGAGCCAA | | |
| 15 | exon #53 | CTTC | UUCCUUAGCU | 942 | AUUGCUCCUUACGAGGAGACUUCC | 1226 | 1368 |
| | | | UCCAGCC | | UUAGCUUCCAGCC | | |
| 16 | exon #53 | CTTC | CUUAGCUUCC | 943 | AUUGCUCCUUACGAGGAGACCUUA | 1227 | 1369 |
| | | | AGCCAUU | | GCUUCCAGCCAUU | | |
| 17 | exon #53 | CTTA | GCUUCCAGCC | 944 | AUUGCUCCUUACGAGGAGACGCUU | 1228 | 1370 |
| | | | AUUGUGU | | CCAGCCAUUGUGU | | |
| 18 | exon #53 | CTTC | CAGCCAUUGU | 945 | AUUGCUCCUUACGAGGAGACCAGC | 1229 | 1371 |
| | | | GUUGAAU | | CAUUGUGUUGAAU | | |
| 19 | exon #53 | ATTC | AACACAAUGG | 946 | AUUGCUCCUUACGAGGAGACAACA | 1230 | 1372 |
| | | | CUGGAAG | | CAAUGGCUGGAAG | | |
| 20 | exon #53 | GTTA | AAGGAUUCAA | 947 | AUUGCUCCUUACGAGGAGACAAGG | 1231 | 1373 |
| | | | CACAAUG | | AUUCAACACAAUG | | |
| 21 | exon #53 | ATTG | UGUUGAAUCC | 948 | AUUGCUCCUUACGAGGAGACUGUU | 1232 | 1374 |
| | | | UUUAACA | | GAAUCCUUUAACA | | |
| 22 | exon #53 | CTTT | AACAUUUCAU | 949 | AUUGCUCCUUACGAGGAGACAACA | 1233 | 1375 |
| | | | UCAACUG | | UUUCAUUCAACUG | | |
| 23 | exon #53 | TTTA | ACAUUUCAUU | 950 | AUUGCUCCUUACGAGGAGACACAU | 1234 | 1376 |
| | | | CAACUGU | | UUCAUUCAACUGU | | |
| 24 | exon #53 | ATTT | CAUUCAACUG | 951 | AUUGCUCCUUACGAGGAGACCAUU | 1235 | 1377 |
| | | | UUGCCUC | | CAACUGUUGCCUC | | |
| 25 | exon #53 | TTTC | AUUCAACUGU | 952 | AUUGCUCCUUACGAGGAGACAUUC | 1236 | 1378 |
| | | | UGCCUCC | | AACUGUUGCCUCC | | |
| 26 | exon #53 | ATTC | AACUGUUGCC | 953 | AUUGCUCCUUACGAGGAGACAACU | 1237 | 1379 |
| | | | UCCGGUU | | GUUGCCUCCGGUU | | |
| 27 | exon #53 | CTTC | AGAACCGGAG | 954 | AUUGCUCCUUACGAGGAGACAGAA | 1238 | 1380 |
| | | | GCAACAG | | CCGGAGGCAACAG | | |
| 28 | exon #53 | GTTG | CCUCCGGUUC | 955 | AUUGCUCCUUACGAGGAGACCCUC | 1239 | 1381 |
| | | | UGAAGGU | | CGGUUCUGAAGGU | | |
| 29 | exon #53 | GTTC | UGAAGGUGU | 956 | AUUGCUCCUUACGAGGAGACUGAA | 1240 | 1382 |
| | | | UCUUGUAC | | GGUGUUCUUGUAC | | |
| 30 | exon #53 | GTTC | UUGUACUUCA | 957 | AUUGCUCCUUACGAGGAGACUUGU | 1241 | 1383 |
| | | | UCCCACU | | ACUUCAUCCCACU | | |
| 31 | exon #53 | CTTG | UACUUCAUCC | 958 | AUUGCUCCUUACGAGGAGACUACU | 1242 | 1384 |
| | | | CACUGAU | | UCAUCCCACUGAU | | |
| 32 | exon #53 | CTTC | AUCCCACUGA | 959 | AUUGCUCCUUACGAGGAGACAUCC | 1243 | 1385 |
| | | | UUCUGAA | | CACUGAUUCUGAA | | |
| 33 | exon #53 | ATTC | AGAAUCAGUG | 960 | AUUGCUCCUUACGAGGAGACAGAA | 1244 | 1386 |
| | | | GGAUGAA | | UCAGUGGGAUGAA | | |
| 34 | exon #52 | ATTG | UUCUAGCCUC | 961 | AUUGCUCCUUACGAGGAGACUUCU | 1245 | 1387 |
| | | | UUGAUUG | | AGCCUCUUGAUUG | | |
| 35 | exon #52 | GTTC | UAGCCUCUUG | 962 | AUUGCUCCUUACGAGGAGACUAGC | 1246 | 1388 |
| | | | AUUGCUG | | CUCUUGAUUGCUG | | |
| 36 | exon #52 | CTTG | AUUGCUGGUC | 963 | AUUGCUCCUUACGAGGAGACAUUG | 1247 | 1389 |
| | | | UUGUUUU | | CUGGUCUUGUUUU | | |
| 37 | exon #52 | ATTG | CUGGUCUUGU | 964 | AUUGCUCCUUACGAGGAGACCUGG | 1248 | 1390 |
| | | | UUUUCAA | | UCUUGUUUUUCAA | | |
| 38 | exon #52 | TTTG | AAAAACAAGA | 965 | AUUGCUCCUUACGAGGAGACAAAA | 1249 | 1391 |
| | | | CCAGCAA | | ACAAGACCAGCAA | | |
| 39 | exon #52 | ATTT | GAAAAACAAG | 966 | AUUGCUCCUUACGAGGAGACGAAA | 1250 | 1392 |
| | | | ACCAGCA | | AACAAGACCAGCA | | |
| 40 | exon #52 | CTTG | UUUUUCAAAU | 967 | AUUGCUCCUUACGAGGAGACUUUU | 1251 | 1393 |
| | | | UUUGGGC | | UCAAAUUUUGGGC | | |
| 41 | exon #52 | GTTT | UUCAAAUUUU | 968 | AUUGCUCCUUACGAGGAGACUUCA | 1252 | 1394 |
| | | | GGGCAGC | | AAUUUUGGGCAGC | | |
| 42 | exon #52 | TTTT | UCAAAUUUUG | 969 | AUUGCUCCUUACGAGGAGACUCAA | 1253 | 1395 |
| | | | GGCAGCG | | AUUUUGGGCAGCG | | |
| 43 | exon #52 | TTTT | CAAAUUUUGG | 970 | AUUGCUCCUUACGAGGAGACCAAA | 1254 | 1396 |
| | | | GCAGCGG | | UUUUGGGCAGCGG | | |
| 44 | exon #52 | TTTC | AAAUUUUGG | 971 | AUUGCUCCUUACGAGGAGACAAAU | 1255 | 1397 |
| | | | GCAGCGGU | | UUUGGGCAGCGGU | | |
| 45 | exon #52 | ATTA | CCGCUGCCCA | 972 | AUUGCUCCUUACGAGGAGACCCGC | 1256 | 1398 |
| | | | AAAUUUG | | UGCCCAAAAUUUG | | |
| 46 | exon #52 | ATTT | UGGGCAGCGG | 973 | AUUGCUCCUUACGAGGAGACUGGG | 1257 | 1399 |
| | | | UAAUGAG | | CAGCGGUAAUGAG | | |
| 47 | exon #52 | TTTT | GGGCAGCGGU | 974 | AUUGCUCCUUACGAGGAGACGGGC | 1258 | 1400 |
| | | | AAUGAGU | | AGCGGUAAUGAGU | | |
| 48 | exon #52 | TTTG | GGCAGCGGUA | 975 | AUUGCUCCUUACGAGGAGACGGCA | 1259 | 1401 |
| | | | AUGAGUU | | GCGGUAAUGAGUU | | |
| 49 | exon #52 | GTTG | GAAGAACUCA | 976 | AUUGCUCCUUACGAGGAGACGAAG | 1260 | 1402 |
| | | | UUACCGC | | AACUCAUUACCGC | | |
| 50 | exon #52 | GTTC | UUCCAACUGG | 977 | AUUGCUCCUUACGAGGAGACUUCC | 1261 | 1403 |
| | | | GGACGCC | | AACUGGGGACGCC | | |
| 51 | exon #52 | CTTC | CAACUGGGGA | 978 | AUUGCUCCUUACGAGGAGACCAAC | 1262 | 1404 |
| | | | CGCCUCU | | UGGGGACGCCUCU | | |
| 52 | exon #52 | TTTG | GAACAGAGGC | 979 | AUUGCUCCUUACGAGGAGACGAAC | 1263 | 1405 |
| | | | GUCCCCA | | AGAGGCGUCCCCA | | |
| 53 | exon #52 | ATTT | GGAACAGAGG | 980 | AUUGCUCCUUACGAGGAGACGGAA | 1264 | 1406 |
| | | | CGUCCCC | | CAGAGGCGUCCCC | | |
| 54 | exon #52 | GTTC | CAAAUCCUGC | 981 | AUUGCUCCUUACGAGGAGACCAAA | 1265 | 1407 |
| | | | AUUGUUG | | UCCUGCAUUGUUG | | |
| 55 | exon #51 | CTTC | UGCUUGAUGA | 982 | AUUGCUCCUUACGAGGAGACUGCU | 1266 | 1408 |
| | | | UCAUCUC | | UGAUGAUCAUCUC | | |
| 56 | exon #51 | CTTG | AUGAUCAUCU | 983 | AUUGCUCCUUACGAGGAGACAUGA | 1267 | 1409 |
| | | | CGUUGAU | | UCAUCUCGUUGAU | | |
| 57 | exon #51 | CTTG | AGGAUAUCAA | 984 | AUUGCUCCUUACGAGGAGACAGGA | 1268 | 1410 |
| | | | CGAGAUG | | UAUCAACGAGAUG | | |
| 58 | exon #51 | GTTG | AUAUCCUCAA | 985 | AUUGCUCCUUACGAGGAGACAUAU | 1269 | 1411 |
| | | | GGUCACC | | CCUCAAGGUCACC | | |
| 59 | exon #51 | GTTA | UAAAAUCACA | 986 | AUUGCUCCUUACGAGGAGACUAAA | 1270 | 1412 |
| | | | GAGGGUG | | AUCACAGAGGGUG | | |
| 60 | exon #51 | ATTT | UAUAACUUGA | 987 | AUUGCUCCUUACGAGGAGACUAUA | 1271 | 1413 |
| | | | UCAAGCA | | ACUUGAUCAAGCA | | |
| 61 | exon #51 | TTTT | AUAACUUGAU | 988 | AUUGCUCCUUACGAGGAGACAUAA | 1272 | 1414 |
| | | | CAAGCAG | | CUUGAUCAAGCAG | | |
| 62 | exon #51 | TTTA | UAACUUGAUC | 989 | AUUGCUCCUUACGAGGAGACUAAC | 1273 | 1415 |
| | | | AAGCAGA | | UUGAUCAAGCAGA | | |
| 63 | exon #51 | TTTC | UCUGCUUGAU | 990 | AUUGCUCCUUACGAGGAGACUCUG | 1274 | 1416 |
| | | | CAAGUUA | | CUUGAUCAAGUUA | | |
| 64 | exon #51 | CTTT | CUCUGCUUGA | 991 | AUUGCUCCUUACGAGGAGACCUCU | 1275 | 1417 |
| | | | UCAAGUU | | GCUUGAUCAAGUU | | |
| 65 | exon #51 | CTTG | AUCAAGCAGA | 992 | AUUGCUCCUUACGAGGAGACAUCA | 1276 | 1418 |
| | | | GAAAGCC | | AGCAGAGAAAGCC | | |
| 66 | exon #51 | CTTA | CCGACUGGCU | 993 | AUUGCUCCUUACGAGGAGACCCGA | 1277 | 1419 |
| | | | UUCUCUG | | CUGGCUUUCUCUG | | |
| 67 | exon #51 | CTTG | GACAGAACUU | 994 | AUUGCUCCUUACGAGGAGACGACA | 1278 | 1420 |
| | | | ACCGACU | | GAACUUACCGACU | | |
| 68 | exon #51 | GTTC | UGUCCAAGCC | 995 | AUUGCUCCUUACGAGGAGACUGUC | 1279 | 1421 |
| | | | CGGUUGA | | CAAGCCCGGUUGA | | |
| 69 | exon #51 | TTTC | AACCGGGCUU | 996 | AUUGCUCCUUACGAGGAGACAACC | 1280 | 1422 |
| | | | GGACAGA | | GGGCUUGGACAGA | | |
| 70 | exon #51 | ATTT | CAACCGGGCU | 997 | AUUGCUCCUUACGAGGAGACCAAC | 1281 | 1423 |
| | | | UGGACAG | | CGGGCUUGGACAG | | |
| 71 | exon #51 | GTTG | AAAUCUGCCA | 998 | AUUGCUCCUUACGAGGAGACAAAU | 1282 | 1424 |
| | | | GAGCAGG | | CUGCCAGAGCAGG | | |
| 72 | exon #51 | GTTG | GAGGUACCUG | 999 | AUUGCUCCUUACGAGGAGACGAGG | 1283 | 1425 |
| | | | CUCUGGC | | UACCUGCUCUGGC | | |
| 73 | exon #51 | CTTG | AUGUUGGAG | 1000 | AUUGCUCCUUACGAGGAGACAUGU | 1284 | 1426 |
| | | | GUACCUGC | | UGGAGGUACCUGC | | |
| 74 | exon #51 | CTTC | CUUGAUGUUG | 1001 | AUUGCUCCUUACGAGGAGACCUUG | 1285 | 1427 |
| | | | GAGGUAC | | AUGUUGGAGGUAC | | |
| 75 | exon #51 | ATTT | CUAGUUUGGA | 1002 | AUUGCUCCUUACGAGGAGACCUAG | 1286 | 1428 |
| | | | GAUGGCA | | UUUGGAGAUGGCA | | |
| 76 | exon #51 | TTTC | UAGUUUGGA | 1003 | AUUGCUCCUUACGAGGAGACUAGU | 1287 | 1429 |
| | | | GAUGGCAG | | UUGGAGAUGGCAG | | |
| 77 | exon #51 | GTTT | GGAGAUGGCA | 1004 | AUUGCUCCUUACGAGGAGACGGAG | 1288 | 1430 |
| | | | GUUUCCU | | AUGGCAGUUUCCU | | |
| 78 | exon #51 | TTTG | GAGAUGGCAG | 1005 | AUUGCUCCUUACGAGGAGACGAGA | 1289 | 1431 |
| | | | UUUCCUU | | UGGCAGUUUCCUU | | |
| 79 | exon #51 | GTTA | CUAAGGAAAC | 1006 | AUUGCUCCUUACGAGGAGACCUAA | 1290 | 1432 |
| | | | UGCCAUC | | GGAAACUGCCAUC | | |
| 80 | exon #51 | GTTT | CCUUAGUAAC | 1007 | AUUGCUCCUUACGAGGAGACCCUU | 1291 | 1433 |
| | | | CACAGGU | | AGUAACCACAGGU | | |
| 81 | exon #51 | TTTC | CUUAGUAACC | 1008 | AUUGCUCCUUACGAGGAGACCUUA | 1292 | 1434 |
| | | | ACAGGUU | | GUAACCACAGGUU | | |
| 82 | exon #51 | CTTA | GUAACCACAG | 1009 | AUUGCUCCUUACGAGGAGACGUAA | 1293 | 1435 |
| | | | GUUGUGU | | CCACAGGUUGUGU | | |
| 83 | exon #51 | GTTA | CUCUGGUGAC | 1010 | AUUGCUCCUUACGAGGAGACCUCU | 1294 | 1436 |
| | | | ACAACCU | | GGUGACACAACCU | | |
| 84 | exon #51 | GTTG | UGUCACCAGA | 1011 | AUUGCUCCUUACGAGGAGACUGUC | 1295 | 1437 |
| | | | GUAACAG | | ACCAGAGUAACAG | | |
| 85 | exon #45 | CTTT | UUUCUGUCUG | 1012 | AUUGCUCCUUACGAGGAGACUUUC | 1296 | 1438 |
| | | | ACAGCUG | | UGUCUGACAGCUG | | |
| 86 | exon #45 | TTTT | UUCUGUCUGA | 1013 | AUUGCUCCUUACGAGGAGACUUCU | 1297 | 1439 |
| | | | CAGCUGU | | GUCUGACAGCUGU | | |
| 87 | exon #45 | TTTT | UCUGUCUGAC | 1014 | AUUGCUCCUUACGAGGAGACUCUG | 1298 | 1440 |
| | | | AGCUGUU | | UCUGACAGCUGUU | | |
| 88 | exon #45 | TTTT | CUGUCUGACA | 1015 | AUUGCUCCUUACGAGGAGACCUGU | 1299 | 1441 |
| | | | GCUGUUU | | CUGACAGCUGUUU | | |
| 89 | exon #45 | TTTC | UGUCUGACAG | 1016 | AUUGCUCCUUACGAGGAGACUGUC | 1300 | 1442 |
| | | | CUGUUUG | | UGACAGCUGUUUG | | |
| 90 | exon #45 | GTTT | GCAGACCUCC | 1017 | AUUGCUCCUUACGAGGAGACGCAG | 1301 | 1443 |
| | | | UGCCACC | | ACCUCCUGCCACC | | |
| 91 | exon #45 | TTTG | CAGACCUCCU | 1018 | AUUGCUCCUUACGAGGAGACCAGA | 1302 | 1444 |
| | | | GCCACCG | | CCUCCUGCCACCG | | |
| 92 | exon #45 | ATTG | GGAAGCCUGA | 1019 | AUUGCUCCUUACGAGGAGACGGAA | 1303 | 1445 |
| | | | AUCUGCG | | GCCUGAAUCUGCG | | |
| 93 | exon #45 | ATTC | AGGCUUCCCA | 1020 | AUUGCUCCUUACGAGGAGACAGGC | 1304 | 1446 |
| | | | AUUUUUC | | UUCCCAAUUUUUC | | |
| 94 | exon #45 | CTTC | CCAAUUUUUC | 1021 | AUUGCUCCUUACGAGGAGACCCAA | 1305 | 1447 |
| | | | CUGUAGA | | UUUUUCCUGUAGA | | |
| 95 | exon #45 | ATTC | UACAGGAAAA | 1022 | AUUGCUCCUUACGAGGAGACUACA | 1306 | 1448 |
| | | | AUUGGGA | | GGAAAAAUUGGGA | | |
| 96 | exon #45 | ATTT | UUCCUGUAGA | 1023 | AUUGCUCCUUACGAGGAGACUUCC | 1307 | 1449 |
| | | | AUACUGG | | UGUAGAAUACUGG | | |
| 97 | exon #45 | TTTT | UCCUGUAGAA | 1024 | AUUGCUCCUUACGAGGAGACUCCU | 1308 | 1450 |
| | | | UACUGGC | | GUAGAAUACUGGC | | |
| 98 | exon #45 | TTTT | CCUGUAGAAU | 1025 | AUUGCUCCUUACGAGGAGACCCUG | 1309 | 1451 |
| | | | ACUGGCA | | UAGAAUACUGGCA | | |
| 99 | exon #45 | TTTC | CUGUAGAAUA | 1026 | AUUGCUCCUUACGAGGAGACCUGU | 1310 | 1452 |
| | | | CUGGCAU | | AGAAUACUGGCAU | | |
| 100 | exon #45 | ATTC | AGCAAUCCUC | 1027 | AUUGCUCCUUACGAGGAGACAGCA | 1311 | 1453 |
| | | | AAAAACA | | AUCCUCAAAAACA | | |
| 101 | exon #45 | GTTT | UUGAGGAUU | 1028 | AUUGCUCCUUACGAGGAGACUUGA | 1312 | 1454 |
| | | | GCUGAAUU | | GGAUUGCUGAAUU | | |
| 102 | exon #45 | TTTT | UGAGGAUUGC | 1029 | AUUGCUCCUUACGAGGAGACUGAG | 1313 | 1455 |
| | | | UGAAUUA | | GAUUGCUGAAUUA | | |
| 103 | exon #45 | TTTT | GAGGAUUGCU | 1030 | AUUGCUCCUUACGAGGAGACGAGG | 1314 | 1456 |
| | | | GAAUUAU | | AUUGCUGAAUUAU | | |
| 104 | exon #45 | TTTG | AGGAUUGCUG | 1031 | AUUGCUCCUUACGAGGAGACAGGA | 1315 | 1457 |
| | | | AAUUAUU | | UUGCUGAAUUAUU | | |
| 105 | exon #45 | ATTG | CUGAAUUAUU | 1032 | AUUGCUCCUUACGAGGAGACCUGA | 1316 | 1458 |
| | | | UCUUCCC | | AUUAUUUCUUCCC | | |
| 106 | exon #45 | ATTA | UUUCUUCCCC | 1033 | AUUGCUCCUUACGAGGAGACUUUC | 1317 | 1459 |
| | | | AGUUGCA | | UUCCCCAGUUGCA | | |
| 107 | exon #45 | ATTT | CUUCCCCAGU | 1034 | AUUGCUCCUUACGAGGAGACCUUC | 1318 | 1460 |
| | | | UGCAUUC | | CCCAGUUGCAUUC | | |
| 108 | exon #45 | TTTC | UUCCCCAGUU | 1035 | AUUGCUCCUUACGAGGAGACUUCC | 1319 | 1461 |
| | | | GCAUUCA | | CCAGUUGCAUUCA | | |
| 109 | exon #45 | ATTG | AAUGCAACUG | 1036 | AUUGCUCCUUACGAGGAGACAAUG | 1320 | 1462 |
| | | | GGGAAGA | | CAACUGGGGAAGA | | |
| 110 | exon #45 | CTTC | CCCAGUUGCA | 1037 | AUUGCUCCUUACGAGGAGACCCCA | 1321 | 1463 |
| | | | UUCAAUG | | GUUGCAUUCAAUG | | |
| 111 | exon #45 | GTTG | CAUUCAAUGU | 1038 | AUUGCUCCUUACGAGGAGACCAUU | 1322 | 1464 |
| | | | UCUGACA | | CAAUGUUCUGACA | | |
| 112 | exon #45 | GTTG | UCAGAACAUU | 1039 | AUUGCUCCUUACGAGGAGACUCAG | 1323 | 1465 |
| | | | GAAUGCA | | AACAUUGAAUGCA | | |
| 113 | exon #45 | ATTC | AAUGUUCUGA | 1040 | AUUGCUCCUUACGAGGAGACAAUG | 1324 | 1466 |
| | | | CAACAGU | | UUCUGACAACAGU | | |
| 114 | exon #45 | GTTC | UGACAACAGU | 1041 | AUUGCUCCUUACGAGGAGACUGAC | 1325 | 1467 |
| | | | UUGCCGC | | AACAGUUUGCCGC | | |
| 115 | exon #45 | ATTG | GGCAGCGGCA | 1042 | AUUGCUCCUUACGAGGAGACGGCA | 1326 | 1468 |
| | | | AACUGUU | | GCGGCAAACUGUU | | |
| 116 | exon #45 | GTTT | GCCGCUGCCC | 1043 | AUUGCUCCUUACGAGGAGACGCCG | 1327 | 1469 |
| | | | AAUGCCA | | CUGCCCAAUGCCA | | |
| 117 | exon #45 | TTTG | CCGCUGCCCA | 1044 | AUUGCUCCUUACGAGGAGACCCGC | 1328 | 1470 |
| | | | AUGCCAU | | UGCCCAAUGCCAU | | |
| 118 | exon #44 | ATTT | GUAUUUAGCA | 1045 | AUUGCUCCUUACGAGGAGACGUAU | 1329 | 1471 |
| | | | UGUUCCC | | UUAGCAUGUUCCC | | |
| 119 | exon #44 | TTTG | UAUUUAGCAU | 1046 | AUUGCUCCUUACGAGGAGACUAUU | 1330 | 1472 |
| | | | GUUCCCA | | UAGCAUGUUCCCA | | |
| 120 | exon #44 | ATTG | GGAACAUGCU | 1047 | AUUGCUCCUUACGAGGAGACGGAA | 1331 | 1473 |
| | | | AAAUACA | | CAUGCUAAAUACA | | |
| 121 | exon #44 | ATTT | AGCAUGUUCC | 1048 | AUUGCUCCUUACGAGGAGACAGCA | 1332 | 1474 |
| | | | CAAUUCU | | UGUUCCCAAUUCU | | |
| 122 | exon #44 | TTTA | GCAUGUUCCC | 1049 | AUUGCUCCUUACGAGGAGACGCAU | 1333 | 1475 |
| | | | AAUUCUC | | GUUCCCAAUUCUC | | |
| 123 | exon #44 | ATTC | CUGAGAAUUG | 1050 | AUUGCUCCUUACGAGGAGACCUGA | 1334 | 1476 |
| | | | GGAACAU | | GAAUUGGGAACAU | | |
| 124 | exon #44 | GTTC | CCAAUUCUCA | 1051 | AUUGCUCCUUACGAGGAGACCCAA | 1335 | 1477 |
| | | | GGAAUUU | | UUCUCAGGAAUUU | | |
| 125 | exon #44 | ATTC | UCAGGAAUUU | 1052 | AUUGCUCCUUACGAGGAGACUCAG | 1336 | 1478 |
| | | | GUGUCUU | | GAAUUUGUGUCUU | | |
| 126 | exon #44 | TTTC | UCAGAAAGAC | 1053 | AUUGCUCCUUACGAGGAGACUCAG | 1337 | 1479 |
| | | | ACAAAUU | | AAAGACACAAAUU | | |
| 127 | exon #44 | ATTT | GUGUCUUUCU | 1054 | AUUGCUCCUUACGAGGAGACGUGU | 1338 | 1480 |
| | | | GAGAAAC | | CUUUCUGAGAAAC | | |
| 128 | exon #44 | GTTT | CUCAGAAAGA | 1055 | AUUGCUCCUUACGAGGAGACCUCA | 1339 | 1481 |
| | | | CACAAAU | | GAAAGACACAAAU | | |
| 129 | exon #44 | TTTG | UGUCUUUCUG | 1056 | AUUGCUCCUUACGAGGAGACUGUC | 1340 | 1482 |
| | | | AGAAACU | | UUUCUGAGAAACU | | |
| 130 | exon #44 | CTTT | CUGAGAAACU | 1057 | AUUGCUCCUUACGAGGAGACCUGA | 1341 | 1483 |
| | | | GUUCAGC | | GAAACUGUUCAGC | | |
| 131 | exon #44 | TTTC | UGAGAAACUG | 1058 | AUUGCUCCUUACGAGGAGACUGAG | 1342 | 1484 |
| | | | UUCAGCU | | AAACUGUUCAGCU | | |
| 132 | exon #44 | GTTC | AGCUUCUGUU | 1059 | AUUGCUCCUUACGAGGAGACAGCU | 1343 | 1485 |
| | | | AGCCACU | | UCUGUUAGCCACU | | |
| 133 | exon #44 | CTTC | UGUUAGCCAC | 1060 | AUUGCUCCUUACGAGGAGACUGUU | 1344 | 1486 |
| | | | UGAUUAA | | AGCCACUGAUUAA | | |
| 134 | exon #44 | TTTA | AUCAGUGGCU | 1061 | AUUGCUCCUUACGAGGAGACAUCA | 1345 | 1487 |
| | | | AACAGAA | | GUGGCUAACAGAA | | |
| 135 | exon #44 | ATTT | AAUCAGUGGC | 1062 | AUUGCUCCUUACGAGGAGACAAUC | 1346 | 1488 |
| | | | UAACAGA | | AGUGGCUAACAGA | | |
| 136 | exon #44 | GTTA | GCCACUGAUU | 1063 | AUUGCUCCUUACGAGGAGACGCCA | 1347 | 1489 |
| | | | AAAUAUC | | CUGAUUAAAUAUC | | |
| 137 | exon #44 | TTTA | UAUCAUAAUG | 1064 | AUUGCUCCUUACGAGGAGACUAUC | 1348 | 1490 |
| | | | AAAACGC | | AUAAUGAAAACGC | | |
| 138 | exon #44 | GTTG | AGAAAUGGCG | 1065 | AUUGCUCCUUACGAGGAGACAGAA | 1349 | 1491 |
| | | | GCGUUUU | | AUGGCGGCGUUUU | | |
| 139 | exon #44 | ATTT | CUCAACAGAU | 1066 | AUUGCUCCUUACGAGGAGACCUCA | 1350 | 1492 |
| | | | CUGUCAA | | ACAGAUCUGUCAA | | |
| 140 | exon #44 | TTTC | UCAACAGAUC | 1067 | AUUGCUCCUUACGAGGAGACUCAA | 1351 | 1493 |
| | | | UGUCAAA | | CAGAUCUGUCAAA | | |
| 141 | exon #44 | TTTG | ACAGAUCUGU | 1068 | AUUGCUCCUUACGAGGAGACACAG | 1352 | 1494 |
| | | | UGAGAAA | | AUCUGUUGAGAAA | | |
| 142 | exon #44 | ATTT | GACAGAUCUG | 1069 | AUUGCUCCUUACGAGGAGACGACA | 1353 | 1495 |
| | | | UUGAGAA | | GAUCUGUUGAGAA | | |

TABLE 9 provide exemplary retRNA sequences designed to pair with CasPhi. 12 and the corresponding gRNA sequences in TABLE 8 with the same targeting site number. The numbering of the targeting sites in TABLE 9 aligns with the numbering in TABLE 8.

TABLE 9
Exemplary CasPhi.12 retRNA sequences

| | | SEQ | | SEQ | retRNA | SEQ | | SEQ |
|---|---|---|---|---|---|---|---|---|
| Target | | ID | | ID | se- | ID | | ID |
| Site | PBS | NO: | RTT | NO: | quence | NO: | retRNA sequence with scaffold region | NO: |
| 27 | CUG | 1496 | UUA | 1520 | UUAAC | 1544 | GUGCUCGCUUCGGCAGCACAUAUACUAGUCGACGGGCCGCA | 1568 |
| | UUG | | ACA | | AUUUC | | CUCGCCGGUCCCAAGCCCGGAUAAAAUGGGAGGGGGCGGGA | |
| | CCU | | UUU | | AUUCA | | AACCGCCUAACCAUGCCGAGUGCGGCCGCAACUAAGCACAU | |
| | CCG | | CAU | | AGUAC | | GAGGAUCACCCAUGUGCUUAACAUUUCAUUCAAGUACCUGU | |
| | GUU | | UCA | | CUGUU | | UGCCUCCGGUUAAAUUAACAGUGGCCGCGGUCGGCGUGGAC | |
| | | | AGU | | GCCUC | | UGUAGAACACUGCCAAUGCCGGUCCCAAGCCCGGAUAAAAG | |
| | | | AC | | CGGUU | | UGGAGGGUACAGUCCACGCUCUAGAGCGGACUUCGGUCCGC | |
| 38 | UUG | 1497 | GUU | 1521 | GUUCU | 1545 | GUGCUCGCUUCGGCAGCACAUAUACUAGUCGACGGGCCGCA | 1569 |
| | CUG | | CUA | | AGCCU | | CUCGCCGGUCCCAAGCCCGGAUAAAAUGGGAGGGGGCGGGA | |
| | GUC | | GCC | | CUUGA | | AACCGCCUAACCAUGCCGAGUGCGGCCGCAACUAAGCACAU | |
| | UUG | | UCU | | GGUAC | | GAGGAUCACCCAUGUGCGUUCUAGCCUCUUGAGGUACUUGC | |
| | UUU | | UGA | | UUGCU | | UGGUCUUGUUUAAAUUAACAGUGGCCGCGGUCGGCGUGGAC | |
| | | | GGU | | GGUCU | | UGUAGAACACUGCCAAUGCCGGUCCCAAGCCCGGAUAAAAG | |
| | | | AC | | UGUUU | | UGGAGGGUACAGUCCACGCUCUAGAGCGGACUUCGGUCCGC | |
| 39 | UGC | 1498 | UUC | 1522 | UUCUA | 1546 | GUGCUCGCUUCGGCAGCACAUAUACUAGUCGACGGGCCGCA | 1570 |
| | UGG | | UAG | | GCCUC | | CUCGCCGGUCCCAAGCCCGGAUAAAAUGGGAGGGGGCGGGA | |
| | UCU | | CCU | | UUGAU | | AACCGCCUAACCAUGCCGAGUGCGGCCGCAACUAAGCACAU | |
| | UGU | | CUU | | GGUAC | | GAGGAUCACCCAUGUGCUUCUAGCCUCUUGAUGGUACUGCU | |
| | UUU | | GAU | | UGCUG | | GGUCUUGUUUUAAAUUAACAGUGGCCGCGGUCGGCGUGGAC | |
| | | | GGU | | GUCUU | | UGUAGAACACUGCCAAUGCCGGUCCCAAGCCCGGAUAAAAG | |
| | | | AC | | GUUUU | | UGGAGGGUACAGUCCACGCUCUAGAGCGGACUUCGGUCCGC | |
| 41 | GCU | 1499 | GAA | 1523 | GAAGA | 1547 | GUGCUCGCUUCGGCAGCACAUAUACUAGUCGACGGGCCGCA | 1571 |
| | GCC | | GAA | | ACUCA | | CUCGCCGGUCCCAAGCCCGGAUAAAAUGGGAGGGGGCGGGA | |
| | CAA | | CUC | | UUACC | | AACCGCCUAACCAUGCCGAGUGCGGCCGCAACUAAGCACAU | |
| | AAU | | AUU | | GGUAC | | GAGGAUCACCCAUGUGCGAAGAACUCAUUACCGGUACGCUG | |
| | UUG | | ACC | | GCUGC | | CCCAAAAUUUGAAAUUAACAGUGGCCGCGGUCGGCGUGGAC | |
| | | | GGU | | CCAAA | | UGUAGAACACUGCCAAUGCCGGUCCCAAGCCCGGAUAAAAG | |
| | | | AC | | AUUUG | | UGGAGGGUACAGUCCACGCUCUAGAGCGGACUUCGGUCCGC | |
| 42 | CGC | 1500 | GGA | 1524 | GGAAG | 1548 | GUGCUCGCUUCGGCAGCACAUAUACUAGUCGACGGGCCGCA | 1572 |
| | UGC | | AGA | | AACUC | | CUCGCCGGUCCCAAGCCCGGAUAAAAUGGGAGGGGGGGCGA | |
| | CCA | | ACU | | AUUAC | | AACCGCCUAACCAUGCCGAGUGCGGCCGCAACUAAGCACAU | |
| | AAA | | CAU | | GGUAC | | GAGGAUCACCCAUGUGCGGAAGAACUCAUUACGGUACCGCU | |
| | UUU | | UAC | | CGCUG | | GCCCAAAAUUUAAAUUAACAGUGGCCGCGGUCGGCGUGGAC | |
| | | | GGU | | CCCAA | | UGUAGAACACUGCCAAUGCCGGUCCCAAGCCCGGAUAAAAG | |
| | | | AC | | AAUUU | | UGGAGGGUACAGUCCACGCUCUAGAGCGGACUUCGGUCCGC | |
| 43 | CCG | 1501 | UGG | 1525 | UGGAA | 1549 | GUGCUCGCUUCGGCAGCACAUAUACUAGUCGACGGGCCGCA | 1573 |
| | CUG | | AAG | | GAACU | | CUCGCCGGUCCCAAGCCCGGAUAAAAUGGGAGGGGGCGGGA | |
| | CCC | | AAC | | CAUUA | | AACCGCCUAACCAUGCCGAGUGCGGCCGCAACUAAGCACAU | |
| | AAA | | UCA | | GGUAC | | GAGGAUCACCCAUGUGCUGGAAGAACUCAUUAGGUACCCGC | |
| | AUU | | UUA | | CCGCU | | UGCCCAAAAUUAAAUUAACAGUGGCCGCGGUCGGCGUGGAC | |
| | | | GGU | | GCCCA | | UGUAGAACACUGCCAAUGCCGGUCCCAAGCCCGGAUAAAAG | |
| | | | AC | | AAAUU | | UGGAGGGUACAGUCCACGCUCUAGAGCGGACUUCGGUCCGC | |
| 44 | ACC | 1502 | UUG | 1526 | UUGGA | 1550 | GUGCUCGCUUCGGCAGCACAUAUACUAGUCGACGGGCCGCA | 1574 |
| | GCU | | GAA | | AGAAC | | CUCGCCGGUCCCAAGCCCGGAUAAAAUGGGAGGGGGCGGGA | |
| | GCC | | GAA | | UCAUU | | AACCGCCUAACCAUGCCGAGUGCGGCCGCAACUAAGCACAU | |
| | CAA | | CUC | | GGUAC | | GAGGAUCACCCAUGUGCUUGGAAGAACUCAUUGGUACACCG | |
| | AAU | | AUU | | ACCGC | | CUGCCCAAAAUAAAUUAACAGUGGCCGCGGUCGGCGUGGAC | |
| | | | GGU | | UGCCC | | UGUAGAACACUGCCAAUGCCGGUCCCAAGCCCGGAUAAAAG | |
| | | | AC | | AAAAU | | UGGAGGGUACAGUCCACGCUCUAGAGCGGACUUCGGUCCGC | |
| 45 | CAA | 1503 | GCU | 1527 | GCUGG | 1551 | GUGCUCGCUUCGGCAGCACAUAUACUAGUCGACGGGCCGCA | 1575 |
| | AUU | | GGU | | UCUUG | | CUCGCCGGUCCCAAGCCCGGAUAAAAUGGGAGGGGGCGGGA | |
| | UUG | | CUU | | UUUUU | | AACCGCCUAACCAUGCCGAGUGCGGCCGCAACUAAGCACAU | |
| | GGC | | GUU | | GGUAC | | GAGGAUCACCCAUGUGCGCUGGUCUUGUUUUUGGUACCAAA | |
| | AGC | | UUU | | CAAAU | | UUUUGGGCAGCAAAUUAACAGUGGCCGCGGUCGGCGUGGAC | |
| | | | GGU | | UUUGG | | UGUAGAACACUGCCAAUGCCGGUCCCAAGCCCGGAUAAAAG | |
| | | | AC | | GCAGC | | UGGAGGGUACAGUCCACGCUCUAGAGCGGACUUCGGUCCGC | |
| 46 | CUC | 1504 | CCC | 1528 | CCCCA | 1552 | GUGCUCGCUUCGGCAGCACAUAUACUAGUCGACGGGCCGCA | 1576 |
| | AUU | | CAG | | GUUGG | | CUCGCCGGUCCCAAGCCCGGAUAAAAUGGGAGGGGGCGGGA | |
| | ACC | | UUG | | AAGAA | | AACCGCCUAACCAUGCCGAGUGCGGCCGCAACUAAGCACAU | |
| | GCU | | GAA | | GGUAC | | GAGGAUCACCCAUGUGCCCCCAGUUGGAAGAAGGUACCUCA | |
| | GCC | | GAA | | CUCAU | | UUACCGCUGCCAAAUUAACAGUGGCCGCGGUCGGCGUGGAC | |
| | | | GGU | | UACCG | | UGUAGAACACUGCCAAUGCCGGUCCCAAGCCCGGAUAAAAG | |
| | | | AC | | CUGCC | | UGGAGGGUACAGUCCACGCUCUAGAGCGGACUUCGGUCCGC | |
| 47 | ACU | 1505 | UCC | 1529 | UCCCC | 1553 | GUGCUCGCUUCGGCAGCACAUAUACUAGUCGACGGGCCGCA | 1577 |
| | CAU | | CCA | | AGUUG | | CUCGCCGGUCCCAAGCCCGGAUAAAAUGGGAGGGGGCGGGA | |
| | UAC | | GUU | | GAAGA | | AACCGCCUAACCAUGCCGAGUGCGGCCGCAACUAAGCACAU | |
| | CGC | | GGA | | GGUAC | | GAGGAUCACCCAUGUGCUCCCCAGUUGGAAGAGGUACACUC | |
| | UGC | | AGA | | ACUCA | | AUUACCGCUGCAAAUUAACAGUGGCCGCGGUCGGCGUGGAC | |
| | | | GGU | | UUACC | | UGUAGAACACUGCCAAUGCCGGUCCCAAGCCCGGAUAAAAG | |
| | | | AC | | GCUGC | | UGGAGGGUACAGUCCACGCUCUAGAGCGGACUUCGGUCCGC | |
| 48 | AAC | 1506 | GUC | 1530 | GUCCC | 1554 | GUGCUCGCUUCGGCAGCACAUAUACUAGUCGACGGGCCGCA | 1578 |
| | UCA | | CCC | | CAGUU | | CUCGCCGGUCCCAAGCCCGGAUAAAAUGGGAGGGGGCGGGA | |
| | UUA | | AGU | | GGAAG | | AACCGCCUAACCAUGCCGAGUGCGGCCGCAACUAAGCACAU | |
| | CCG | | UGG | | GGUAC | | GAGGAUCACCCAUGUGCGUCCCCAGUUGGAAGGGUACAACU | |
| | CUG | | AAG | | AACUC | | CAUUACCGCUGAAAUUAACAGUGGCCGCGGUCGGCGUGGAC | |
| | | | GGU | | AUUAC | | UGUAGAACACUGCCAAUGCCGGUCCCAAGCCCGGAUAAAAG | |
| | | | AC | | CGCUG | | UGGAGGGUACAGUCCACGCUCUAGAGCGGACUUCGGUCCGC | |
| 49 | GCG | 1507 | UUC | 1531 | UUCAA | 1555 | GUGCUCGCUUCGGCAGCACAUAUACUAGUCGACGGGCCGCA | 1579 |
| | GUA | | AAA | | AUUUU | | CUCGCCGGUCCCAAGCCCGGAUAAAAUGGGAGGGGGCGGGA | |
| | AUG | | UUU | | GGGCA | | AACCGCCUAACCAUGCCGAGUGCGGCCGCAACUAAGCACAU | |
| | AGU | | UGG | | GGUAC | | GAGGAUCACCCAUGUGCUUCAAAUUUUGGGCAGGUACGCGG | |
| | UCU | | GCA | | GCGGU | | UAAUGAGUUCUAAAUUAACAGUGGCCGCGGUCGGCGUGGAC | |
| | | | GGU | | AAUGA | | UGUAGAACACUGCCAAUGCCGGUCCCAAGCCCGGAUAAAAG | |
| | | | AC | | GUUCU | | UGGAGGGUACAGUCCACGCUCUAGAGCGGACUUCGGUCCGC | |
| 50 | GGC | 1508 | AGG | 1532 | AGGAU | 1556 | GUGCUCGCUUCGGCAGCACAUAUACUAGUCGACGGGCCGCA | 1580 |
| | GUC | | AUU | | UUGGA | | CUCGCCGGUCCCAAGCCCGGAUAAAAUGGGAGGGGGCGGGA | |
| | CCC | | UGG | | ACAGA | | AACCGCCUAACCAUGCCGAGUGCGGCCGCAACUAAGCACAU | |
| | AGU | | AAC | | GGUAC | | GAGGAUCACCCAUGUGCAGGAUUUGGAACAGAGGUACGGCG | |
| | UGG | | AGA | | GGCGU | | UCCCCAGUUGGAAAUUAACAGUGGCCGCGGUCGGCGUGGAC | |
| | | | GGU | | CCCCA | | UGUAGAACACUGCCAAUGCCGGUCCCAAGCCCGGAUAAAAG | |
| | | | AC | | GUUGG | | UGGAGGGUACAGUCCACGCUCUAGAGCGGACUUCGGUCCGC | |
| 51 | AGA | 1509 | UGC | 1533 | UGCAG | 1557 | GUGCUCGCUUCGGCAGCACAUAUACUAGUCGACGGGCCGCA | 1581 |
| | GGC | | AGG | | GAUUU | | CUCGCCGGUCCCAAGCCCGGAUAAAAUGGGAGGGGGCGGGA | |
| | GUC | | AUU | | GGAAC | | AACCGCCUAACCAUGCCGAGUGCGGCCGCAACUAAGCACAU | |
| | CCC | | UGG | | GGUAC | | GAGGAUCACCCAUGUGCUGCAGGAUUUGGAACGGUACAGAG | |
| | AGU | | AAC | | AGAGG | | GCGUCCCCAGUAAAUUAACAGUGGCCGCGGUCGGCGUGGAC | |
| | | | GGU | | CGUCC | | UGUAGAACACUGCCAAUGCCGGUCCCAAGCCCGGAUAAAAG | |
| | | | AC | | CCAGU | | UGGAGGGUACAGUCCACGCUCUAGAGCGGACUUCGGUCCGC | |
| 52 | UGG | 1510 | AUG | 1534 | AUGAG | 1558 | GUGCUCGCUUCGGCAGCACAUAUACUAGUCGACGGGCCGCA | 1582 |
| | GGA | | AGU | | UUCUU | | CUCGCCGGUCCCAAGCCCGGAUAAAAUGGGAGGGGGCGGGA | |
| | CGC | | UCU | | CCAAC | | AACCGCCUAACCAUGCCGAGUGCGGCCGCAACUAAGCACAU | |
| | CUC | | UCC | | GGUAC | | GAGGAUCACCCAUGUGCAUGAGUUCUUCCAACGGUACUGGG | |
| | UGU | | AAC | | UGGGG | | GACGCCUCUGUAAAUUAACAGUGGCCGCGGUCGGCGUGGAC | |
| | | | GGU | | ACGCC | | UGUAGAACACUGCCAAUGCCGGUCCCAAGCCCGGAUAAAAG | |
| | | | AC | | UCUGU | | UGGAGGGUACAGUCCACGCUCUAGAGCGGACUUCGGUCCGC | |
| 53 | GGG | 1511 | UGA | 1535 | UGAGU | 1559 | GUGCUCGCUUCGGCAGCACAUAUACUAGUCGACGGGCCGCA | 1583 |
| | | | GUU | | UCUUC | | CUCGCCGGUCCCAAGCCCGGAUAAAAUGGGAGGGGGCGGGA | |
| | GAC | | CUU | | CAACU | | AACCGCCUAACCAUGCCGAGUGCGGCCGCAACUAAGCACAU | |
| | GCC | | CCA | | GGUAC | | GAGGAUCACCCAUGUGCUGAGUUCUUCCAACUGGUACGGGG | |
| | UCU | | ACU | | GGGGA | | ACGCCUCUGUUAAAUUAACAGUGGCCGCGGUCGGCGUGGAC | |
| | GUU | | GGU | | CGCCU | | UGUAGAACACUGCCAAUGCCGGUCCCAAGCCCGGAUAAAAG | |
| | | | AC | | CUGUU | | UGGAGGGUACAGUCCACGCUCUAGAGCGGACUUCGGUCCGC | |
| 54 | CAA | 1512 | AUU | 1536 | AUUUG | 1560 | GUGCUCGCUUCGGCAGCACAUAUACUAGUCGACGGGCCGCA | 1584 |
| | CAA | | UGU | | UUCUU | | CUCGCCGGUCCCAAGCCCGGAUAAAAUGGGAGGGGGCGGGA | |
| | UGC | | UCU | | ACAGG | | AACCGCCUAACCAUGCCGAGUGCGGCCGCAACUAAGCACAU | |
| | AGG | | UAC | | GGUAC | | GAGGAUCACCCAUGUGCAUUUGUUCUUACAGGGGUACCAAC | |
| | AUU | | AGG | | CAACA | | AAUGCAGGAUUAAAUUAACAGUGGCCGCGGUCGGCGUGGAC | |
| | | | GGU | | AUGCA | | UGUAGAACACUGCCAAUGCCGGUCCCAAGCCCGGAUAAAAG | |
| | | | AC | | GGAUU | | UGGAGGGUACAGUCCACGCUCUAGAGCGGACUUCGGUCCGC | |
| 77 | AGG | 1513 | CAA | 1537 | CAACC | 1561 | GUGCUCGCUUCGGCAGCACAUAUACUAGUCGACGGGCCGCA | 1585 |
| | AAA | | CCU | | UGUGG | | CUCGCCGGUCCCAAGCCCGGAUAAAAUGGGAGGGGGCGGGA | |
| | CUG | | GUG | | UUACU | | AACCGCCUAACCAUGCCGAGUGCGGCCGCAACUAAGCACAU | |
| | CCA | | GUU | | AGUAC | | GAGGAUCACCCAUGUGCCAACCUGUGGUUACUAGUACAGGA | |
| | UCU | | ACU | | AGGAA | | AACUGCCAUCUAAAUUAACAGUGGCCGCGGUCGGCGUGGAC | |
| | | | AGU | | ACUGC | | UGUAGAACACUGCCAAUGCCGGUCCCAAGCCCGGAUAAAAG | |
| | | | AC | | CAUCU | | UGGAGGGUACAGUCCACGCUCUAGAGCGGACUUCGGUCCGC | |
| 78 | AAG | 1514 | ACA | 1538 | ACAAC | 1562 | GUGCUCGCUUCGGCAGCACAUAUACUAGUCGACGGGCCGCA | 1586 |
| | GAA | | ACC | | CUGUG | | CUCGCCGGUCCCAAGCCCGGAUAAAAUGGGAGGGGGCGGGA | |
| | ACU | | UGU | | GUUAC | | AACCGCCUAACCAUGCCGAGUGCGGCCGCAACUAAGCACAU | |
| | GCC | | GGU | | UGUAC | | GAGGAUCACCCAUGUGCACAACCUGUGGUUACUGUACAAGG | |
| | AUC | | UAC | | AAGGA | | AAACUGCCAUCAAAUUAACAGUGGCCGCGGUCGGCGUGGAC | |
| | | | UGU | | AACUG | | UGUAGAACACUGCCAAUGCCGGUCCCAAGCCCGGAUAAAAG | |
| | | | AC | | CCAUC | | UGGAGGGUACAGUCCACGCUCUAGAGCGGACUUCGGUCCGC | |
| 84 | CUG | 1515 | UUA | 1539 | UUAGC | 1563 | GUGCUCGCUUCGGCAGCACAUAUACUAGUCGACGGGCCGCA | 1587 |
| | UUA | | GCU | | UCCUA | | CUCGCCGGUCCCAAGCCCGGAUAAAAUGGGAGGGGGCGGGA | |
| | CUC | | CCU | | CUCAG | | AACCGCCUAACCAUGCCGAGUGCGGCCGCAACUAAGCACAU | |
| | UGG | | ACU | | AGUAC | | GAGGAUCACCCAUGUGCUUAGCUCCUACUCAGAGUACCUGU | |
| | UGA | | CAG | | CUGUU | | UACUCUGGUGAAAAUUAACAGUGGCCGCGGUCGGCGUGGAC | |
| | | | AGU | | ACUCU | | UGUAGAACACUGCCAAUGCCGGUCCCAAGCCCGGAUAAAAG | |
| | | | AC | | GGUGA | | UGGAGGGUACAGUCCACGCUCUAGAGCGGACUUCGGUCCGC | |
| 108 | UGA | 1516 | ACU | 1540 | ACUGU | 1564 | GUGCUCGCUUCGGCAGCACAUAUACUAGUCGACGGGCCGCA | 1588 |
| | AUG | | GUU | | UGUCA | | CUCGCCGGUCCCAAGCCCGGAUAAAAUGGGAGGGGGCGGGA | |
| | CAA | | GUC | | GAACA | | AACCGCCUAACCAUGCCGAGUGCGGCCGCAACUAAGCACAU | |
| | CUG | | AGA | | UGUAC | | GAGGAUCACCCAUGUGCACUGUUGUCAGAACAUGUACUGAA | |
| | GGG | | ACA | | UGAAU | | UGCAACUGGGGAAAUUAACAGUGGCCGCGGUCGGCGUGGAC | |
| | | | UGU | | GCAAC | | UGUAGAACACUGCCAAUGCCGGUCCCAAGCCCGGAUAAAAG | |
| | | | AC | | UGGGG | | UGGAGGGUACAGUCCACGCUCUAGAGCGGACUUCGGUCCGC | |
| 114 | GCG | 1517 | AGG | 1541 | AGGAU | 1565 | GUGCUCGCUUCGGCAGCACAUAUACUAGUCGACGGGCCGCA | 1589 |
| | GCA | | AUG | | GGCAU | | CUCGCCGGUCCCAAGCCCGGAUAAAAUGGGAGGGGGCGGGA | |
| | AAC | | GCA | | UGGGC | | AACCGCCUAACCAUGCCGAGUGCGGCCGCAACUAAGCACAU | |
| | UGU | | UUG | | AGUAC | | GAGGAUCACCCAUGUGCAGGAUGGCAUUGGGCAGUACGCGG | |
| | UGU | | GGC | | GCGGC | | CAAACUGUUGUAAAUUAACAGUGGCCGCGGUCGGCGUGGAC | |
| | | | AGU | | AAACU | | UGUAGAACACUGCCAAUGCCGGUCCCAAGCCCGGAUAAAAG | |
| | | | AC | | GUUGU | | UGGAGGGUACAGUCCACGCUCUAGAGCGGACUUCGGUCCGC | |
| 116 | UGG | 1518 | UAC | 1542 | UACAG | 1566 | GUGCUCGCUUCGGCAGCACAUAUACUAGUCGACGGGCCGCA | 1590 |
| | CAU | | AGG | | GAACU | | CUCGCCGGUCCCAAGCCCGGAUAAAAUGGGAGGGGGCGGGA | |
| | UGG | | AAC | | CCAGG | | AACCGCCUAACCAUGCCGAGUGCGGCCGCAACUAAGCACAU | |
| | GCA | | UCC | | AGUAC | | GAGGAUCACCCAUGUGCUACAGGAACUCCAGGAGUACUGGC | |
| | GCG | | AGG | | UGGCA | | AUUGGGCAGCGAAAUUAACAGUGGCCGCGGUCGGCGUGGAC | |
| | | | AGU | | UUGGG | | UGUAGAACACUGCCAAUGCCGGUCCCAAGCCCGGAUAAAAG | |
| | | | AC | | CAGCG | | UGGAGGGUACAGUCCACGCUCUAGAGCGGACUUCGGUCCGC | |
| 117 | AUG | 1519 | UUA | 1543 | UUACA | 1567 | GUGCUCGCUUCGGCAGCACAUAUACUAGUCGACGGGCCGCA | 1591 |
| | GCA | | CAG | | GGAAC | | CUCGCCGGUCCCAAGCCCGGAUAAAAUGGGAGGGGGCGGGA | |
| | UUG | | GAA | | UCCAG | | AACCGCCUAACCAUGCCGAGUGCGGCCGCAACUAAGCACAU | |
| | GGC | | CUC | | GGUAC | | GAGGAUCACCCAUGUGCUUACAGGAACUCCAGGGUACAUGG | |
| | AGC | | CAG | | AUGGC | | CAUUGGGCAGCAAAUUAACAGUGGCCGCGGUCGGCGUGGAC | |
| | | | GGU | | AUUGG | | UGUAGAACACUGCCAAUGCCGGUCCCAAGCCCGGAUAAAAG | |
| | | | AC | | GCAGC | | UGGAGGGUACAGUCCACGCUCUAGAGCGGACUUCGGUCCGC | |

TABLE 10 provides Moloney murine leukemia virus (MMLV) reverse transcriptase sequences.

TABLE 10
Exemplary MMLV sequences

| | | SEQ |
|---|---|---|
| Protein | | ID |
| Name | Sequence | NO: |
| MMLV | TLNIEDEYRLHETSKEPDVSLGSTWLSDFPQAWAE | 1592 |
| | TGGMGLAVRQAPLIIPLKATSTPVSIKQYPMSQEA | |
| | RLGIKPHIQRLLDQGILVPCQSPWNTPLLPVKKPG | |
| | TNDYRPVQDLREVNKRVEDIHPTVPNPYNLLSGLP | |
| | PSHQWYTVLDLKDAFFCLRLHPTSQPLFAFEWRDP | |
| | EMGISGQLTWTRLPQGFKNSPTLFDEALHRDLADF | |
| | RIQHPDLILLQYVDDLLLAATSELDCQQGTRALLQ | |
| | TLGNLGYRASAKKAQICQKQVKYLGYLLKEGQRWL | |
| | TEARKETVMGQPTPKTPRQLREFLGTAGFCRLWIP | |
| | GFAEMAAPLYPLTKTGTLFNWGPDQQKAYQEIKQA | |
| | LLTAPALGLPDLTKPFELFVDEKQGYAKGVLTQKL | |
| | GPWRRPVAYLSKKLDPVAAGWPPCLRMVAAIAVLT | |
| | KDAGKLTMGQPLVILAPHAVEALVKQPPDRWLSNA | |
| | RMTHYQALLLDTDRVQFGPVVALNPATLLPLPEEG | |
| | LQHNCLDILAEAHGTRPDLTDQPLPDADHTWYTDG | |
| | SSLLQEGQRKAGAAVTTETEVIWAKALPAGTSAQR | |
| | AELIALTQALKMAEGKKLNVYTDSRYAFATAHIHG | |
| | EIYRRRGLLTSEGKEIKNKDEILALLKALFLPKRL | |
| | SIIHCPGHQKGHSAEARGNRMADQAARKAAITETP | |
| | DTSTLLIENSSP | |
| MMLV | TLNIEDEYRLHETSKEPDVSLGSTWLSDFPQAWAE | 1593 |
| | TGGMGLAVRQAPLIIPLKATSTPVSIKQYPMSQEA | |
| | RLGIKPHIQRLLDQGILVPCQSPWNTPLLPVKKPG | |
| | TNDYRPVQDLREVNKRVEDIHPTVPNPYNLLSGLP | |
| | PSHQWYTVLDLKDAFFCLRLHPTSQPLFAFEWRDP | |
| | EMGISGQLTWTRLPQGFKNSPTLFNEALHRDLADF | |
| | RIQHPDLILLQYVDDLLLAATSELDCQQGTRALLQ | |
| | TLGNLGYRASAKKAQICQKQVKYLGYLLKEGQRWL | |
| | TEARKETVMGQPTPKTPRQLREFLGKAGFCRLFIP | |
| | GFAEMAAPLYPLTKPGTLFNWGPDQQKAYQEIKQA | |
| | LLTAPALGLPDLTKPFELFVDEKQGYAKGVLTQKL | |
| | GPWRRPVAYLSKKLDPVAAGWPPCLRMVAAIAVLT | |
| | KDAGKLTMGQPLVILAPHAVEALVKQPPDRWLSNA | |
| | RMTHYQALLLDTDRVQFGPVVALNPATLLPLPEEG | |
| | LQHNCLDILAEAHGTRPDLTDQPLPDADHTWYTDG | |
| | SSLLQEGQRKAGAAVTTETEVIWAKALPAGTSAQR | |
| | AELIALTQALKMAEGKKLNVYTDSRYAFATAHIHG | |
| | EIYRRRGWLTSEGKEIKNKDEILALLKALFLPKRL | |
| | SIIHCPGHQKGHSAEARGNRMADQAARKAAITETP | |
| | DTSTLLIENSSP | |
| MMLV RT | TLNIEDEYRLHETSKEPDVSLGSTWLSDFPQAWAE | 1609 |
| for | TGGMGLAVRQAPLIIPLKATSTPVSIKQYPMSQEA | |
| Example | RLGIKPHIQRLLDQGILVPCQSPWNTPLLPVKKPG | |
| 10 | TNDYRPVQDLREVNKRVEDIHPTVPNPYNLLSGLP | |
| | PSHQWYTVLDLKDAFFCLRLHPTSQPLFAFEWRDP | |
| | EMGISGQLTWTRLPQGFKNSPTLFNEALHRDLADF | |
| | RIQHPDLILLQYVDDLLLAATSELDCQQGTRALLQ | |
| | TLGNLGYRASAKKAQICQKQVKYLGYLLKEGQRWL | |
| | TEARKETVMGQPTPKTPRQLREFLGKAGFCRLFIP | |
| | GFAEMAAPLYPLTKPGTLFNWGPDQQKAYQEIKQA | |
| | LLTAPALGLPDLTKPFELFVDEKQGYAKGVLTQKL | |
| | GPWRRPVAYLSKKLDPVAAGWPPCLRMVAAIAVLT | |
| | KDAGKLTMGQPLVILAPHAVEALVKQPPDRWLSNA | |
| | RMTHYQALLLDTDRVQFGPVVALNPATLLPLPEEG | |
| | LQHNCLDILAEAHGTRPDLTDQPLPDADHTWYTDG | |
| | SSLLQEGQRKAGAAVTTETEVIWAKALPAGTSAQR | |
| | AELIALTQALKMAEGKKLNVYTDSRYAFATAHIHG | |
| | EIYRRRGWLTSEGKEIKNKDEILALLKALFLPKRL | |
| | SIIHCPGHQKGHSAEARGNRMADQAARKAAITETP | |
| | DTSTLLIENSSPSGGSKRTADGSEFES | |

TABLE 11 provides nCas9 (H840A) sequence.

TABLE 11
nCas9(H840A) sequences

| | | SEQ |
|---|---|---|
| Protein | | ID |
| Name | Sequence | NO: |
| nCas9 | DKKYSIGLDIGTNSVGWAVITDEYKVPSKKFKVLG | 1594 |
| (H840A) | NTDRHSIKKNLIGALLFDSGETAEATRLKRTARRR | |
| | YTRRKNRICYLQEIFSNEMAKVDDSFFHRLEESFL | |
| | VEEDKKHERHPIFGNIVDEVAYHEKYPTIYHLRKK | |
| | LVDSTDKADLRLIYLALAHMIKFRGHFLIEGDLNP | |
| | DNSDVDKLFIQLVQTYNQLFEENPINASGVDAKAI | |
| | LSARLSKSRRLENLIAQLPGEKKNGLFGNLIALSL | |
| | GLTPNFKSNFDLAEDAKLQLSKDTYDDDLDNLLAQ | |
| | IGDQYADLFLAAKNLSDAILLSDILRVNTEITKAP | |
| | LSASMIKRYDEHHQDLTLLKALVRQQLPEKYKEIF | |
| | FDQSKNGYAGYIDGGASQEEFYKFIKPILEKMDGT | |
| | EELLVKLNREDLLRKQRTFDNGSIPHQIHLGELHA | |
| | ILRRQEDFYPFLKDNREKIEKILTFRIPYYVGPLA | |
| | RGNSRFAWMTRKSEETITPWNFEEVVDKGASAQSF | |
| | IERMTNFDKNLPNEKVLPKHSLLYEYFTVYNELTK | |
| | VKYVTEGMRKPAFLSGEQKKAIVDLLFKTNRKVTV | |
| | KQLKEDYFKKIECFDSVEISGVEDRFNASLGTYHD | |
| | LLKIIKDKDFLDNEENEDILEDIVLTLTLFEDREM | |
| | IEERLKTYAHLFDDKVMKQLKRRRYTGWGRLSRKL | |
| | INGIRDKQSGKTILDFLKSDGFANRNFMQLIHDDS | |
| | LTFKEDIQKAQVSGQGDSLHEHIANLAGSPAIKKG | |
| | ILQTVKVVDELVKVMGRHKPENIVIEMARENQTTQ | |
| | KGQKNSRERMKRIEEGIKELGSQILKEHPVENTQL | |
| | QNEKLYLYYLQNGRDMYVDQELDINRLSDYDVDAI | |
| | VPQSFLKDDSIDNKVLTRSDKNRGKSDNVPSEEVV | |
| | KKMKNYWRQLLNAKLITQRKFDNLTKAERGGLSEL | |
| | DKAGFIKRQLVETRQITKHVAQILDSRMNTKYDEN | |
| | DKLIREVKVITLKSKLVSDFRKDFQFYKVREINNY | |
| | HHAHDAYLNAVVGTALIKKYPKLESEFVYGDYKVY | |
| | DVRKMIAKSEQEIGKATAKYFFYSNIMNFFKTEIT | |
| | LANGEIRKRPLIETNGETGEIVWDKGRDFATVRKV | |
| | LSMPQVNIVKKTEVQTGGFSKESILPKRNSDKLIA | |
| | RKKDWDPKKYGGFDSPTVAYSVLVVAKVEKGKSKK | |
| | LKSVKELLGITIMERSSFEKNPIDFLEAKGYKEVK | |
| | KDLIIKLPKYSLFELENGRKRMLASAGELQKGNEL | |
| | ALPSKYVNFLYLASHYEKLKGSPEDNEQKQLFVEQ | |
| | HKHYLDEIIEQISEFSKRVILADANLDKVLSAYNK | |
| | HRDKPIREQAENIIHLFTLTNLGAPAAFKYFDTTI | |
| | DRKRYTSTKEVLDATLIHQSITGLYETRIDLSQLG | |
| | GD | |

TABLE 12 provides XTEN40) linker sequence.

TABLE 12
XTEN40 linker sequences

| Protein | Protein | | SEQ ID |
|---|---|---|---|
| Name | Type | Sequence | NO: |
| XTEN40 | Linker | GSSGGSPAGSPTSTEEGTSE | 1595 |
| linker | | SATPESGPGTSTEPSEGSAP | |
| | | GSPAGSGGGS | |

TABLE 13
Exemplary diseases, disorders and syndromes
Exemplary Diseases and Syndromes

Muscular Dystrophy (MD); Muscular Dystrophy, Duchenne Type (DMD); Dilated Cardiomyopathy (DCM) Type 3B; Duchenne muscular
dystrophy; Muscular Dystrophy; Muscular Dystrophy, Becker Type (BMD); Dystrophinopathies; Familial Isolated Dilated Cardiomyopathy;
Dilated Cardiomyopathy; Myopathy; Colorectal Cancer; Isolated Elevated Serum Creatine Phosphokinase Levels; Atrial Standstill 1; Creatine
Phosphokinase, Elevated Serum; Neuromuscular Disease; Atrial Heart Septal Defect; Arrhythmogenic Right Ventricular Cardiomyopathy;
Heart Disease; Glycerol Kinase Deficiency; Non-Syndromic X-Linked Intellectual Disability; Respiratory Failure; Rhabdomyosarcoma;
Miyoshi Muscular Dystrophy; Scoliosis; Facioscapulohumeral Muscular Dystrophy 1; Ptosis; Hypertrophic Cardiomyopathy; Schizophrenia;
Myositis; Autism; Myocarditis; Muscular Dystrophy, Limb-Girdle, Autosomal Recessive 2; Adrenal Hypoplasia, Congenital; Autosomal
Recessive Limb-Girdle Muscular Dystrophy; Restrictive Cardiomyopathy; Walker-Warburg Syndrome; Muscular Dystrophy, Congenital,
Lmna-Related; Centronuclear Myopathy; Cataract; Spinal Muscular Atrophy; Long Qt Syndrome; Emery-Dreifuss Muscular Dystrophy;
Retinitis Pigmentosa; Malignant Hyperthermia; Pectus Excavatum; Brugada Syndrome; Myoglobinuria; Muscular Dystrophy-
Dystroglycanopathy, Type a, 4; Muscle Hypertrophy; Cardiomyopathy, Familial Hypertrophic, 1; Batten-Turner Congenital Myopathy;
Bethlem Myopathy 1; Eye Disease; Aland Island Eye Disease; Glycogen Storage Disease II; Left Ventricular Noncompaction; Glycogen
Storage Disease; Brody Myopathy; Myofibrillar Myopathy; Beckwith-Wiedemann Syndrome; Polyglucosan Body Myopathy 1 with or Without
Immunodeficiency; Interatrial Communication; Congenital Fiber-Type Disproportion; Chromosome Xp21 Deletion Syndrome; Multiple
Pterygium Syndrome, Escobar Variant; Lissencephaly; Rigid Spine Muscular Dystrophy 1; Hypertrophic Pyloric Stenosis; Progressive
Muscular Dystrophy; X-Linked Recessive Disease; Muscular Disease; Disease of Mental Health; Exophthalmos; Gas Gangrene; Symptomatic
Form of Muscular Dystrophy of Duchenne and Becker in Female Carriers; Muscular Atrophy; Qualitative or Quantitative Defects of
Dystrophin; Muscular Dystrophy, Congenital Merosin-Deficient, 1a; Autosomal Recessive Limb-Girdle Muscular Dystrophy Type 2c; Mcleod
Syndrome; Muscular Dystrophy, Duchenne and Becker Type; Autosomal Recessive Limb-Girdle Muscular Dystrophy Type 2d; Ullrich
Congenital Muscular Dystrophy 1; Nr0b1-Related Adrenal Hypoplasia Congenita; Waardenburg Syndrome, Type 4b; Nonaka Myopathy;
Intrinsic Cardiomyopathy; Autosomal Recessive Limb-Girdle Muscular Dystrophy Type 2b; Myotonic Dystrophy 1; Muscular Dystrophy,
Limb-Girdle, Autosomal Recessive 6; Emery-Dreifuss Muscular Dystrophy 2, Autosomal Dominant; Muscular Dystrophy, Limb-Girdle,
Autosomal Recessive 7; Myopathy, Myofibrillar, 3; Myopathy, Myofibrillar, 5; Myopathy, Myofibrillar, 1; Peripheral Nervous System Disease;
Muscle Eye Brain Disease; Cardiomyopathy, Familial Hypertrophic, 4; Microcolon; Hemophagocytic Lymphohistiocytosis, Familial, 1;
Autosomal Recessive Limb-Girdle Muscular Dystrophy Type 2a; Tibial Muscular Dystrophy; Congenital Muscular Dystrophy-
Dystroglycanopathy Type a; Bone Structure Disease; Autosomal Recessive Limb-Girdle Muscular Dystrophy Type 2f; Immunodeficiency 26;
Oculomedin; Cardioneuromyopathy with Hyaline Masses and Nemaline Rods; Keratosis Follicularis Spinulosa Decalvans, X-Linked;
Myoglobinuria, Recurrent; Muscular Dystrophy-Dystroglycanopathy; X-Linked Monogenic Disease; Muscle Tissue Disease; Fundus
Dystrophy; Interstitial Myocarditis; Localized Lipodystrophy; Extracardiac Rhabdomyoma; Cytoplasmic Body Myopathy; Autosomal
Dominant Distal Myopathy; Reducing Body Myopathy; Cobblestone Lissencephaly; Multiple Sclerosis; Barrett Esophagus; Gastric Cancer,
Hereditary Diffuse; Colorectal Cancer 12; Polyposis Syndrome, Hereditary Mixed, 1; Small Intestine Adenocarcinoma; Esophageal Cancer;
Esophagus Squamous Cell Carcinoma; Cardiomyopathy, Dilated, 1b; Limb-Girdle Muscular Dystrophy; 48, xyyy; 48, xxxy; 48, xxyy; Alacrima,
Achalasia, and Mental Retardation Syndrome; 49, xyyyy; 49, xxxxx; 49, xxxxy; 47, xyy; Lmna-Related Dilated Cardiomyopathy; Lung
Combined Type Small Cell Carcinoma; Lung Occult Small Cell Carcinoma; Lung Non-Squamous Non-Small Cell Carcinoma;
Cardiomyopathy, Dilated, 1a; Cardiomyopathy, Dilated, 1h; Cardiac Conduction Defect; Meningioma, Familial; Congestive Heart Failure;
Myotonic Dystrophy; Breast Cancer; Severe Combined Immunodeficiency; Dysphagia; Fibrosis of Extraocular Muscles, Congenital, 1;
Muscular Dystrophy, Limb-Girdle, Autosomal Recessive 5; Autism Spectrum Disorder; Polykaryocytosis Inducer; Progressive Familial Heart
Block; Heart Conduction Disease; Relapsing-Remitting Multiple Sclerosis; Colon Adenocarcinoma; Glioblastoma; Encephalopathy,
Progressive, Early-Onset, with Episodic Rhabdomyolysis; Metabolic Crises, Recurrent, with Rhabdomyolysis, Cardiac Arrhythmias, and
Neurodegeneration; Attention Deficit-Hyperactivity Disorder; Neuroretinitis; Retinitis; Learning Disability; Secondary Progressive Multiple
Sclerosis; Myotonia Congenita, Autosomal Recessive; Endomyocardial Fibrosis; Pik3ca-Related Overgrowth Syndrome; Genetic
Neuromuscular Disease; Short Stature-Obesity Syndrome; Hypoadrenocorticism, Familial; Cleft Palate, Isolated; Osteoporosis; Bone Mineral
Density Quantitative Trait Locus 8; Bone Mineral Density Quantitative Trait Locus 15; Gonadal Dysgenesis; Turner Syndrome; Hypoxia;
Phenylketonuria; Brugada Syndrome 1; Neuronal Migration Disorders; Cardiac Arrest; Hypotonia; Amyotrophic Lateral Sclerosis 1; Glioma
Susceptibility 1; Lateral Sclerosis; Corneal Edema; Polymyositis; Sleep Disorder; Cleft Lip; Muscular Dystrophy, Limb-Girdle, Autosomal
Recessive 1; Glioma Susceptibility 9; Glioma Susceptibility 2; Glioma Susceptibility 3; Pilocytic Astrocytoma; Diarrhea; Hemophilia; Chronic
Granulomatous Disease; Cleft Lip with or Without Cleft Palate; Type 2 Diabetes Mellitus; Triiodothyronine Receptor Auxiliary Protein;
Macroglossia; Melanoma; Orofaciodigital Syndrome I; Orofaciodigital Syndrome; Aging; Rapidly Involuting Congenital Hemangioma;
Sensorineural Hearing Loss; Yemenite Deaf-Blind Hypopigmentation Syndrome; Toxic Encephalopathy; West Syndrome; Gastrointestinal
Stromal Tumor; Osteogenic Sarcoma; Skeletal Muscle Disease; Intracranial Meningioma; Muscular Dystrophy-Dystroglycanopathy, Type C,
5; Ataxia, Combined Cerebellar and Peripheral, with Hearing Loss and Diabetes Mellitus; Branchiootic Syndrome 1; Deafness, X-Linked 3;
Secretory Meningioma; Lymphoplasmacyte-Rich Meningioma; Factor Viii Deficiency; Hemophilia a; Dermatomyositis; Calpain-3-Related
Limb-Girdle Muscular Dystrophy R1; Qualitative or Quantitative Defects of Alpha-Dystroglycan; Congenital Muscular Dystrophy Due to
Dystroglycanopathy; Growth Hormone Deficiency; Cleft Lip/palate; Parkinsonism; Microcephaly; Cerebral Palsy; Osteomalacia; Bosma
Arhinia Microphthalmia Syndrome; Intraocular Pressure Quantitative Trait Locus; Combined Immunodeficiency; Maple Syrup Urine Disease;
Papillomatosis, Confluent and Reticulated; Peutz-Jeghers Syndrome; Rippling Muscle Disease 2; Muscular Dystrophy-Dystroglycanopathy,
Type B, 5; Nonsyndromic 46, xx Testicular Disorders of Sex Development; Hand Skill, Relative; Orofacial Cleft; Retinal Detachment;
Constipation; Sarcoma; Spindle Cell Sarcoma; Premature Menopause; Sleep Apnea; Dysferlinopathy; Qualitative or Quantitative Defects of
Dysferlin; Autonomic Dysfunction; Graft-Versus-Host Disease; Microphthalmia, Syndromic 10; Chondroblastoma; Bone Mineral Density
Quantitative Trait Locus 3; Leukemia, Acute Lymphoblastic; Methane Production; Ischemia; Idiopathic Scoliosis; Alcohol Dependence;
Premature Ovarian Failure 1; Demyelinating Disease; Qualitative or Quantitative Defects of Sarcoglycan; Mitral Valve Insufficiency;
Myopathy, X-Linked, with Excessive Autophagy; Mucositis; Inclusion Body Myositis; Dystonia; Bone Resorption Disease; Body Mass Index
Quantitative Trait Locus 1; Neuritis; Cystic Fibrosis; Polycystic Kidney Disease; Charcot-Marie-Tooth Disease; Myasthenia Gravis; Helix
Syndrome; Hyperinsulinism; Lipid Metabolism Disorder; Tooth Disease; Lung Disease; Muscular Dystrophy, Limb-Girdle, Autosomal
Recessive 3; Miyoshi Muscular Dystrophy 1; Pseudohyperkalemia, Familial, 2, Due to Red Cell Leak; Urinary Tract Infection; Early-Onset
Generalized Limb-Onset Dystonia; Multinucleated Neurons, Anhydramnios, Renal Dysplasia, Cerebellar Hypoplasia, and Hydranencephaly;
Agenesis of Corpus Callosum, Cardiac, Ocular, and Genital Syndrome; Pneumothorax; Proteasome-Associated Autoinflammatory Syndrome 1;
Pancreatic Cancer; Gastric Cancer; Neuromyelitis Optica; Open-Angle Glaucoma; Disease by Infectious Agent; Resting Heart Rate, Variation
in; Poliomyelitis; Childhood Type Dermatomyositis; Progressive Multifocal Leukoencephalopathy; Swallowing Disorders; Premature Aging;
Rickets; Hirschsprung Disease, Cardiac Defects, and Autonomic Dysfunction; Optic Neuritis; Progressive Muscular Atrophy; Spinal Muscular
Atrophy, Type Ii; Glucose Intolerance; Nephrolithiasis; Hypogonadism; Motor Neuron Disease; Congenital Muscular Dystrophy Type 1a;
Autoimmune Disease; Atrioventricular Block; Glucocorticoid-Induced Osteoporosis; Epilepsy; Back Pain; Fragile X Syndrome; B-
Lymphoblastic Leukemia/lymphoma; Mitochondrial Myopathy; Dyslexia; Ataxia-Telangiectasia; Obsessive-Compulsive Disorder; Torticollis;
Proteinuria, Chronic Benign; Pulmonary Fibrosis; Myotonia; Metabolic Acidosis; Brittle Bone Disorder; Scoliosis, Isolated 1; Vascular
Disease; Bilirubin Metabolic Disorder; Night Blindness; Chromosomal Triplication; Dentinogenesis Imperfecta Type 2; Astigmatism; Severe
Acute Respiratory Syndrome; Telangiectasis; Skin Disease; Microphthalmia; Myopathy, Distal, with Anterior Tibial Onset;
Hyperhomocysteinemia; Congenital Stationary Night Blindness; Hypothyroidism; Mitral Valve Disease; Leiomyosarcoma; Nemaline
Myopathy; Distal Muscular Dystrophy with Anterior Tibial Onset; Diabetes Mellitus; Influenza; Herpes Simplex; Juvenile Rheumatoid
Arthritis; Central Sleep Apnea; Homocysteinemia; Mitochondrial Disorders; Nervous System Disease; Keratoconus; Nasopharyngitis;
Glaucoma, Primary Open Angle; Central Centrifugal Cicatricial Alopecia; Anxiety; Dermatitis, Atopic; Aphasia; Sexual Disorder; Acute
Cystitis; Dermatitis; Kidney Disease; Tetanus; Myopia; Hypokalemia; Spinal Cord Injury; Cyanide Poisoning; Cardiogenic Shock; Huntington
Disease; Spinal Muscular Atrophy, Type I; Colitis; Down Syndrome; Hair Whorl; Achondroplasia; Apnea, Obstructive Sleep; Barth Syndrome;
Autosomal Recessive Disease; Hepatitis B; Movement Disease; Hemosiderosis; Hepatitis; Gastric Dilatation; Teratoma; Gastroparesis;
Progressive Familial Heart Block, Type Ia; Chondroma; Neurofibromatosis; Hyperthyroidism; Enchondroma; Pulmonary Embolism;
Hypoglycemia; Rare Hereditary Hemochromatosis; Paresthesia; Charcot-Marie-Tooth Disease, Demyelinating, Type 1a; Keratitis, Hereditary;
Insulin-Like Growth Factor I; Drug Allergy; Body Mass Index Quantitative Trait Locus 8; Body Mass Index Quantitative Trait Locus 14; Body
Mass Index Quantitative Trait Locus 18; Body Mass Index Quantitative Trait Locus 7; Body Mass Index Quantitative Trait Locus 4; Body
Mass Index Quantitative Trait Locus 10; Orthostatic Intolerance; Body Mass Index Quantitative Trait Locus 12; Vitreoretinopathy, Neovascular
Inflammatory; Abetalipoproteinemia; Body Mass Index Quantitative Trait Locus 11; Body Mass Index Quantitative Trait Locus 9; Ichthyosis,
X-Linked; Lymphoma; Ocular Albinism; Cervical Dystonia; Uveitis; Hydrocephalus; Liver Cirrhosis; Acute Myocarditis; Skin Carcinoma;
Ichthyosis; Hypertensive Heart Disease; Hypogonadotropic Hypogonadism; Hepatocellular Carcinoma; Body Mass Index Quantitative Trait
Locus 19; Pulmonary Hypertension; Albinism; B-Cell Lymphoma; Allergic Encephalomyelitis; Cytokine Deficiency; Vitreoretinopathy;
Prader-Willi Syndrome; Oculopharyngeal Muscular Dystrophy; Neurodegeneration with Brain Iron Accumulation 2a; Spondylometaphyseal
Dysplasia, Sedaghatian Type; Aspiration Pneumonia; Human Immunodeficiency Virus Type 1; Arcus Corneae; Taqi Polymorphism;
Inflammatory Bowel Disease; Epidermolysis Bullosa; Temporal Lobe Epilepsy; Blepharospasm; Corneal Neovascularization; Neuroaxonal
Dystrophy; Neurodevelopmental, Jaw, Eye, and Digital Syndrome; Blistering, Acantholytic, of Oral and Laryngeal Mucosa; Collagen Vi-
Related Dystrophies; Waardenburg's Syndrome; Fasting Hypoglycemia; X-Linked Congenital Stationary Night Blindness; Malignant
Hyperthermia Susceptibility; Tremor; Aneurysm; Chronic Pain; Thalassemia; Leukemia, Chronic Lymphocytic; Netherton Syndrome; Sjogren
Syndrome; Perlman Syndrome; Rothmund-Thomson Syndrome, Type 2; Clostridium Difficile Colitis; Covid-19; Pulmonary Fibrosis,
Idiopathic; Muscular Dystrophy, Limb-Girdle, Autosomal Recessive 4; Poikiloderma with Neutropenia; Myopathy, Proximal, with
Ophthalmoplegia; Glucocorticoid Resistance, Generalized; Alstrom Syndrome; Intellectual Developmental Disorder, X-Linked 21; Cognitive
Function 1, Social; Medulloblastoma; Danon Disease; Limb Ischemia; Dementia; Conjunctivitis; Neutropenia; Ehlers-Danlos Syndrome;
Polyneuropathy; Phaeohyphomycosis; Vaginal Discharge; Hyperglycemia; Bullous Keratopathy; Keratopathy; Acute Disseminated
Encephalomyelitis; Thyroiditis; Soft Tissue Sarcoma; Pathologic Nystagmus; Pachygyria; Depression; Overgrowth Syndrome; Optic Atrophy
1; Muscular Dystrophy-Dystroglycanopathy, Type a, 1; Hypertriglyceridemia 1; Schwartz-Jampel Syndrome, Type 1; Moyamoya Disease 1;
Left Bundle Branch Hemiblock; Usher Syndrome; Thrombotic Thrombocytopeniaurpura; Neural Tube Defects; Fibrodysplasia Ossificans
Progressiva; Gastroesophageal Reflux; Meniere Disease; Diamond-Blackfan Anemia 2; Night Blindness, Congenital Stationary, Type 1a;
Retinoschisis 1, X-Linked, Juvenile; Retinitis Pigmentosa-Deafness Syndrome; Type 1 Diabetes Mellitus; Acute Kidney Failure; Leukemia;
Epidermolysis Bullosa Dystrophica; Pneumonia; Osteochondrodysplasia; Purpura; Interstitial Lung Disease; Heart Septal Defect; Compartment
Syndrome; Acne; Adenoma; Ileus; Chronic Kidney Disease; Mitochondrial Encephalomyopathy; Measles; Ltbp4-Related Cutis Laxa;
Spinocerebellar Degeneration; Nonsyndromic Hearing Loss; Primary Adrenal Insufficiency; Leukemia, Chronic Lymphocytic 2;
Pseudoachondroplasia; Blood Group--Kell System; Cardiomyopathy, Familial Hypertrophic, 2; Central Core Disease of Muscle;
Rhabdomyosarcoma 2; Human Cytomegalovirus Infection; Frontometaphyseal Dysplasia; Cholelithiasis; Congenital Hypothyroidism;
Hypophosphatemia; Juvenile Glaucoma; Epicanthus; Exudative Vitreoretinopathy 1; Charcot-Marie-Tooth Disease, Axonal, Type 2e;
Progressive Familial Heart Block, Type Ib; Chorea, Childhood-Onset, with Psychomotor Retardation; Myopathy, Distal, with Rimmed
Vacuoles; Deafness, X-Linked 1; Frontometaphyseal Dysplasia 1; Budd-Chiari Syndrome; Myocardial Infarction; Ectodermal Dysplasia-
Syndactyly Syndrome 2; Alpha/beta T-Cell Lymphopenia with Gamma/delta T-Cell Expansion, Severe Cytomegalovirus Infection, and
Autoimmunity; Cardiomyopathy, Dilated, 1x; Apnea, Central Sleep; Adrenal Hyperplasia, Congenital, Due to 21-Hydroxylase Deficiency;
Thyroid Carcinoma, Familial Medullary; Intellectual Developmental Disorder, X-Linked 29; Orofaciodigital Syndrome Viii; Myopathy,
Centronuclear, X-Linked; Progressive Relapsing Multiple Sclerosis; Duane Retraction Syndrome; Choreatic Disease; Hemopericardium;
Cardiac Tamponade; Right Bundle Branch Block; Candidiasis; Pseudohermaphroditism; Laryngitis; Multiple Endocrine Neoplasia; Tricuspid
Valve Insufficiency; Mouth Disease; Superior Mesenteric Artery Syndrome; Histiocytosis; Pericardial Effusion; Clubfoot; Testicular Disease;
Thyroid Gland Medullary Carcinoma; Cerebrovascular Disease; Mitochondrial Metabolism Disease; Hypertrichosis; Acute Myocardial
Infarction; Skin Melanoma; Dyskinesia of Esophagus; Dysautonomia; Congenital Hydrocephalus; Athetosis; Progeroid Syndrome; Muscular
Lipidosis; Laminin Subunit Alpha 2-Related Congenital Muscular Dystrophy; Cerebrofacial Arteriovenous Metameric Syndrome; Isolated
Duane Retraction Syndrome; Thyroid Carcinoma; Arteries, Anomalies of; Lipoid Congenital Adrenal Hyperplasia; Multiple System Atrophy 1;
Chediak-Higashi Syndrome; Pneumothorax, Primary Spontaneous; Lymphoproliferative Syndrome; Pre-Eclampsia; Myelodysplastic
Syndrome; Familial Adenomatous Polyposis; Myopathy, Lactic Acidosis, and Sideroblastic Anemia; Hashimoto Thyroiditis; Muscular
Dystrophy-Dystroglycanopathy, Type C, 4; 46, xy Sex Reversal 2; Cardiomyopathy, Dilated, 1g; Sickle Cell Anemia; Muscular Dystrophy,
Limb-Girdle, Autosomal Recessive 10; Ataxia and Polyneuropathy, Adult-Onset; Leukemia, Acute Myeloid; Macular Degeneration, Age-
Related, 1; Muscular Dystrophy, Limb-Girdle, Autosomal Dominant 3; Aspergillosis; Muscular Dystrophy, Limb-Girdle, Autosomal Dominant
1; Polydactyly; Methylmalonic Acidemia and Homocysteinemia, Cblx Type; Rett Syndrome; Adenomyosis; Myotonic Dystrophy 2; Intellectual
Developmental Disorder, X-Linked 41; Incontinentia Pigmenti; Ornithine Transcarbamylase Deficiency, Hyperammonemia Due to; Coffin-
Lowry Syndrome; Mucopolysaccharidosis, Type Ii; Paralytic Poliomyelitis; Primary Progressive Multiple Sclerosis; Neuronal Ceroid
Lipofuscinosis; Mood Disorder; Postpoliomyelitis Syndrome; Avoidant Personality Disorder; Personality Disorder; Dysentery; Guillain-Barre
Syndrome; Basilar Artery Occlusion; Squamous Cell Papilloma; Megacolon; Hyperuricemia; Lactic Acidosis; Hermaphroditism; Toxic
Megacolon; T Cell Deficiency; Goiter; Retinal Ischemia; Inguinal Hernia; Thrombosis; Craniosynostosis with Fibular Aplasia; Retinal Vascular
Disease; Papilloma; Sensory Peripheral Neuropathy; Lipoprotein Quantitative Trait Locus; Fibromyalgia; Overnutrition; Liver Disease; Peptic
Ulcer Disease; Interstitial Keratitis; Sideroblastic Anemia; Juvenile Retinoschisis; Limb-Girdle Muscular Dystrophy Type 1a; Pattern
Dystrophy; Pellucid Marginal Degeneration; X-Linked Congenital Retinoschisis; 46, Xy Disorders of Sexual Development; Limb-Girdle
Muscular Dystrophy Type 1b; Limb-Girdle Muscular Dystrophy Type 1c; Genetic Skeletal Muscle Disease; Ventilator-Induced Diaphragmatic
Dysfunction; Mesial Temporal Lobe Epilepsy with Hippocampal Sclerosis; Acute Adrenal Insufficiency; Atherosclerosis Susceptibility;
Noonan Syndrome 1; Ovarian Cancer; Dowling-Degos Disease 1; Lymphatic Malformation 5; Antigen Defined by Monoclonal Antibody Aj9;
Myopathy, Congenital, with Fiber-Type Disproportion; Obesity-Hypoventilation Syndrome; Ocular Motor Apraxia; Mitochondrial Complex I
Deficiency, Nuclear Type 1; Muscular Dystrophy, Limb-Girdle, Autosomal Recessive 8; Respiratory Distress Syndrome in Premature Infants;
Bacterial Infectious Disease; Actn3 Deficiency; Tetralogy of Fallot; Sarcoidosis 1; Parkinson Disease, Late-Onset; Progressive External
Ophthalmoplegia with Mitochondrial Dna Deletions, Autosomal Dominant 1; Myopathy, Tubular Aggregate, 1; Chromosome 3q29 Deletion
Syndrome; Progressive External Ophthalmoplegia with Mitochondrial Dna Deletions, Autosomal Dominant 4; Salih Myopathy; Major
Depressive Disorder; Methylmalonic Aciduria and Homocystinuria, Cblc Type; Retinitis Pigmentosa 3; Progressive External Ophthalmoplegia
with Mitochondrial Dna Deletions, Autosomal Dominant 2; Nemaline Myopathy 1; Glut1 Deficiency Syndrome 2; Nasopharyngeal Carcinoma;
Dengue Virus; Peripartum Cardiomyopathy; Impaired Intellectual Development and Distinctive Facial Features with or Without Cardiac
Defects; Hemophilia B; Malaria; Hamamy Syndrome; Night Blindness, Congenital Stationary, Type 1e; Mucopolysaccharidosis-Plus
Syndrome; Mental Retardation, Autosomal Dominant 7; Beta-Thalassemia; Breasts and/or Nipples, Aplasia or Hypoplasia of, 1; Kearns-Sayre
Syndrome; Retinitis Pigmentosa 11; Stroke, Ischemic; Menkes Disease; Nemaline Myopathy 3; Linear Skin Defects with Multiple Congenital
Anomalies 1; Third-Degree Atrioventricular Block; Tracheomalacia; Ifap Syndrome 2; Severe Pre-Eclampsia; Adenocarcinoma; Squamous
Cell Carcinoma; Childhood Absence Epilepsy; Dysthymic Disorder; Cholera; Anal Squamous Cell Carcinoma; Adenosine Deaminase
Deficiency; Posterior Myocardial Infarction; Lymphopenia; Thrombocytopenia; Graves' Disease; Chronic Progressive External
Ophthalmoplegia; Newborn Respiratory Distress Syndrome; Primary Biliary Cholangitis; Olivopontocerebellar Atrophy; Gastroenteritis; Optic
Nerve Disease; Enthesopathy; Focal Epilepsy; Mental Depression; Fibrosarcoma; Placental Insufficiency; Cystic Lymphangioma; Egg Allergy;
Rhinitis; Intracranial Embolism; Neurilemmoma; Mesenchymal Cell Neoplasm; Middle East Respiratory Syndrome; Eclampsia; Autosomal
Dominant Non-Syndromic Intellectual Disability 5; Epidermolysis Bullosa Simplex with Muscular Dystrophy; Peripheral Vascular Disease;
Angina Pectoris; Prion Disease; Neuroblastoma; Viral Infectious Disease; Cholangitis; in Situ Carcinoma; Collagen Disease; Polyhydramnios;
Atp7a-Related Copper Transport Disorders; Dyrkla Syndrome; Grin1-Related Neurodevelopmental Disorder; Cap Myopathy; Splenomegaly;
Gigantism; Iqsec2; Pseudo-Turner Syndrome; Neuropathy; Chilaiditi Syndrome; Childhood-Onset Nemaline Myopathy; Broken Heart
Syndrome; Syngap1-Related Intellectual Disability; Homologous Wasting Disease; Necrotizing Autoimmune Myopathy; Methylmalonic
Acidemia with Homocystinuria; Encephalitis; Intermediate Congenital Nemaline Myopathy; Paroxysmal Exertion-Induced Dyskinesia;
Pediatric Multiple Sclerosis; Hypereosinophilic Syndrome; Specific Language Disorder; Periodic Paralysis; Univentricular Heart; Qualitative or
Quantitative Defects of Beta-Sarcoglycan; Acute Generalized Exanthematous Pustulosis; Disorder of Copper Metabolism; Headache;
Tracheobronchomalacia; Seizure Disorder; Metabolic Myopathy; Inclusion Body Myopathy with Early-Onset Paget Disease with or Without
Frontotemporal Dementia 1; Prostate Cancer; Volvulus of Midgut; Acyl-Coa Dehydrogenase, Very Long-Chain, Deficiency of; Arachnoid
Cysts, Intracranial; Bladder Cancer; Candidiasis, Familial, 1; Cone-Rod Dystrophy 2; Jalili Syndrome; Schopf-Schulz-Passarge Syndrome;
Carpal Tunnel Syndrome; Clubfoot, Congenital, with or Without Deficiency of Long Bones and/or Mirror-Image Polydactyly; Retinoblastoma;
Kabuki Syndrome 1; Migraine with or Without Aura 1; Leprosy 3; Myosclerosis, Autosomal Recessive; Nemaline Myopathy 2; Sialuria; Fryns
Syndrome; Dihydrolipoamide Dehydrogenase Deficiency; D-Bifunctional Protein Deficiency; Periodontitis, Chronic; Exanthem; Peripheral
Artery Disease; Pontocerebellar Hypoplasia; 3-Methylglutaconic Aciduria; Mitochondrial Dna Depletion Syndrome; Neurofibromatosis, Type
I; Hemophagocytic Lymphohistiocytosis; Chromosome 16p11.2 Deletion Syndrome; Syndromic X-Linked Intellectual Disability Snyder Type;
Intestinal Pseudo-Obstruction; Leber Plus Disease; Non-Syndromic X-Linked Intellectual Disability 2; Strabismus; Exostoses, Multiple, Type I;
Gnathodiaphyseal Dysplasia; Nondisjunction; Hypercholesterolemia, Familial, 1; Cholestasis, Intrahepatic, of Pregnancy, 1; Leiomyoma,
Uterine; Frontotemporal Dementia and/or Amyotrophic Lateral Sclerosis 1; Facial Spasm; Kleefstra Syndrome 1; Muscular Dystrophy, Limb-
Girdle, Autosomal Recessive 12; Cavitary Optic Disc Anomalies; Wilson Disease; Night Blindness, Congenital Stationary, Type 2a; Thiamine
Metabolism Dysfunction Syndrome 2; Nemaline Myopathy 4; Meningioma, Radiation-Induced; Accelerated Tumor Formation; Arthrogryposis,
Mental Retardation, and Seizures; Muscular Dystrophy-Dystroglycanopathy, Type C, 7; Tremor, Hereditary Essential, 5; Developmental and
Epileptic Encephalopathy 75; Neuronal Ceroid-Lipofuscinoses; Mandibular Hypoplasia, Deafness, Progeroid Features, and Lipodystrophy
Syndrome; Encephalopathy, Progressive, Early-Onset, with Brain Edema and/or Leukoencephalopathy, 1; Mycobacterium Tuberculosis 1;
Hypophosphatemic Rickets, X-Linked Recessive; Fragile X Tremor/ataxia Syndrome; Pigmentary Disorder, Reticulate, with Systemic
Manifestations, X-Linked; Nance-Horan Syndrome; Chondrodysplasia Punctata 2, X-Linked Dominant; Choroideremia; Paget Disease of Bone
2, Early-Onset; Aromatic L-Amino Acid Decarboxylase Deficiency; Cervical Cancer; Muscular Dystrophy-Dystroglycanopathy, Type a, 7;
Cardiomyopathy, Dilated, lii; Mitochondrial Dna Depletion Syndrome 13; 3-Methylglutaconic Aciduria, Type Vii; Muscular Dystrophy-
Dystroglycanopathy, Type B, 2; Premature Ovarian Failure 7; Muscular Dystrophy-Dystroglycanopathy, Type C, 2; Frontotemporal Dementia
and/or Amyotrophic Lateral Sclerosis 6; Muscular Dystrophy-Dystroglycanopathy, Type a, 2; Canavan Disease; Thymoma, Familial;
Citrullinemia, Type Ii, Adult-Onset; Cardiomyopathy, Familial Restrictive, 3; Lesch-Nyhan Syndrome; Severe Combined Immunodeficiency,
X-Linked; Intellectual Developmental Disorder, X-Linked, Syndromic, Snyder-Robinson Type; Pyruvate Dehydrogenase E1-Alpha Deficiency;
Chondrosarcoma; Perrault Syndrome 1; Cholestasis, Progressive Familial Intrahepatic, 2; Fryns Microphthalmia Syndrome; Cardiomyopathy,
Dilated, 1d; Macular Degeneration, X-Linked Atrophic; Developmental and Epileptic Encephalopathy 1; Renpenning Syndrome 1; Prostatic
Hyperplasia, Benign; X Inactivation, Familial Skewed, 1; Adrenoleukodystrophy; Pettigrew Syndrome; Intellectual Developmental Disorder,
X-Linked, Syndromic, Lujan-Fryns Type; Ubiquitin-Activating Enzyme, Y-Linked; Tooth Agenesis; Coenzyme Q10 Deficiency Disease;
Perrault Syndrome; Tongue Squamous Cell Carcinoma; Oral Squamous Cell Carcinoma; Syndromic X-Linked Intellectual Disability 14;
Progressive Familial Intrahepatic Cholestasis; Gynecomastia; Leiomyoma; Color Blindness; Prostatic Adenoma; Paraplegia; Demyelinating
Polyneuropathy; Essential Tremor; Chronic Inflammatory Demyelinating Polyradiculoneuropathy; Detrusor Sphincter Dyssynergia; Familial
Hypercholesterolemia; Angioedema; Cerebral Degeneration; Gaucher's Disease; Amelogenesis Imperfecta; Thymoma; Olfactory
Neuroblastoma; Transient Cerebral Ischemia; Aortic Aneurysm; Junctional Epidermolysis Bullosa; Malignant Astrocytoma; Complex Regional
Pain Syndrome; Neuromuscular Junction Disease; Rectosigmoid Cancer; Macular Retinal Edema; Systemic Scleroderma; Skull Base
Meningioma; Corneal Dystrophy; Kidney Cancer; Prostatic Hypertrophy; Adult Respiratory Distress Syndrome; Pulmonary Edema;
Hemiplegia; Infant Gynecomastia; Congenital Muscular Dystrophy-Dystroglycanopathy A7; Congenital Muscular Dystrophy-
Dystroglycanopathy Type A2; Non-Alcoholic Steatohepatitis; Cystinosis; Osteopetrosis; Achromatopsia; Allergic Disease; Atrial Fibrillation;
Lung Cancer; Panniculitis; Urticaria; Dental Caries; Cholestasis; Gastritis; Axonal Neuropathy; Clear Cell Meningioma; Acquired
Immunodeficiency Syndrome; Retinal Degeneration; Vasculitis; Mesenchymal Chondrosarcoma; Biotin-Thiamine-Responsive Basal Ganglia
Disease; Free Sialic Acid Storage Disorders; Giant Axonal Neuropathy; Chronic Fatigue Syndrome; Amyloidosis; Periodontitis;
Stenotrophomonas Maltophilia Infection; Encephalopathy; Congenital Nystagmus; Hereditary Neuropathies; Coccygodynia; Glioma; Anca-
Associated Vasculitis; Auriculo-Condylar Syndrome; Chromosome Xp Deletion; Corticobasal Degeneration; Mechanical Strabismus;
Trichorhinophalangeal Syndrome; Adrenomyeloneuropathy; Exencephaly; Hansens Disease; Precocious Puberty; Madelung Deformity; Ocular
Albinism, X-Linked; Rrm2b Mitochondrial Dna Maintenance Defects; Diabetic Neuropathy; Glial Tumor; Familial Intrahepatic Cholestasis;
Spastic Paraplegia-Paget Disease of Bone Syndrome; Rigid Spine Muscular Dystrophy; Traumatic Brain Injury; Laminin Subunit Alpha 2-
Related Muscular Dystrophy; Ring Chromosome; Homozygous Familial Hypercholesterolemia; Cerebral Aneurysms; Anoxia; Cerebral
Hypoxia; Color Vision Deficiency; Laminopathy; Familial Isolated Restrictive Cardiomyopathy; Polyploidy; Argyria; or Non-Syndromic
Pontocerebellar Hypoplasia

### EXAMPLES

The following examples are included for illustrative purposes only and are not intended to limit the scope of the disclosure.

### Example 1

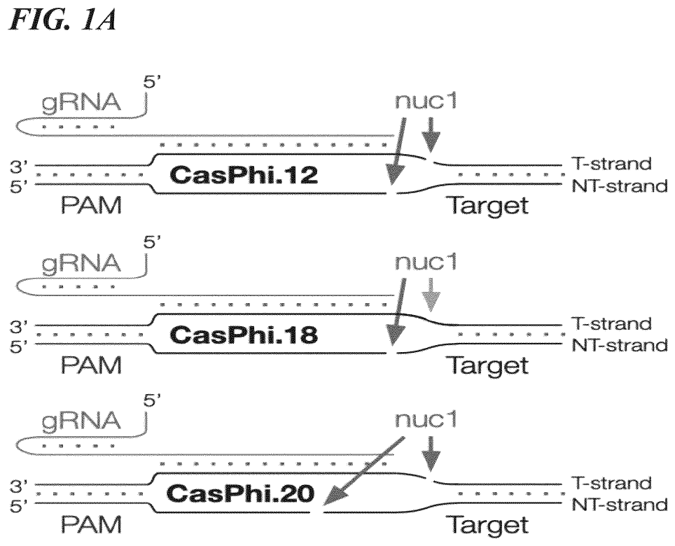

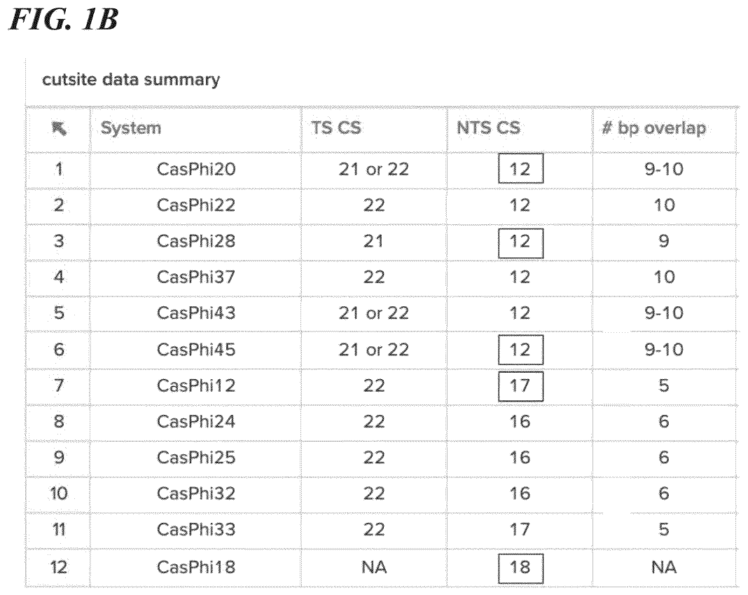

Without limiting ourselves to any particular method or mechanism, effector proteins of the instant disclosure may cleave the non-target strand and/or the target strand of a dsDNA target nucleic acid, see, e.g., FIG. 1A. A ribonucleic acid protein (RNP) complex of the effector protein and guide nucleic acid recognizes a PAM on the target DNA and binds the dsDNA target nucleic acid. A spacer sequence of the guide nucleic acid hybridizes to a target sequence of the dsDNA target nucleic acid. The RNP nicks the target strand and/or the non-target strand of the dsDNA target nucleic acid and the strands of the target DNA dissociate at, or near, the location of nicking. Different effector proteins may nick the nontarget and target strands at different locations and thereby cause an overlap of the target and non-target strands (see, e.g., FIG. 1B). For example, CasPhi.12 (SEQ ID NO: 2) cleaves the non-target strand at 17 bps 3′ of the PAM sequence (that is located on the target strand) and cleaves the target strand at 22 bps 3′ of the PAM sequence. This results in a 5 bp overlap.

### Example 2

An extended guide RNA (rtgRNA) is oriented starting at the 5′ end with a template sequence which is complementary to the target sequence on the non-target strand with the exception of one or more nucleotides conveying an intended genomic edit, and a primer binding sequence (PBS). The template sequence may be referred to as an “RT template.” Located 3′ of the PBS, there is a linker connecting the PBS to the protein binding region comprising a repeat sequence. Lastly, there is the spacer sequence that targets the extended guide RNA and an effector protein-RDDP fusion protein to the target sequence (e.g., genomic DNA). See, e.g., FIG. 3. The effector protein nicks the non-target strand of the target nucleic acid and the RDDP synthesizes new DNA off of the nicked end, wherein the new DNA is complementary to the RT template, thereby producing the desired genomic edit in the target nucleic acid.

### Example 3

An extended guide RNA (rtgRNA) is oriented starting at the 5′ end with a protein binding region comprising a repeat sequence, followed by a region comprising a spacer sequence that hybridizes to a target sequence of DNA. Located 3′ of the region comprising the spacer sequence is a template sequence (e.g., an “RT template”) that is complementary to the target sequence on the non-target strand with the exception of one or more nucleotides conveying an intended edit of the target nucleic acid (e.g., genomic DNA). Located 3′ of the RT template is a primer binding sequence (PBS). The effector protein is linked to RDDP so that when the extended guide RNA complexes with the target sequence the effector protein and the RDDP are both localized to the target sequence of the target nucleic acid. See, e.g., FIG. 4. The effector protein nicks the non-target strand of the target nucleic acid and the RDDP synthesizes new DNA off of the nicked end, wherein the new DNA is complementary to the RT template, thereby producing the desired genomic edit in the target nucleic acid.

### Example 4

A gene editing system comprises two separate RNAs and/or two separate proteins. Such systems may be referred to as split protein/RNA design systems. The system comprises a guide RNA, which comprises a spacer sequence that is complementary to a target sequence on a target strand of a target nucleic acid (e.g., genomic DNA, dsDNA molecule). The guide RNA also comprises a protein binding region comprising a repeat sequence. The protein binding region is bound by an effector protein. The repeat sequence may be directly or indirectly 5′ of the spacer sequence. In other instances, the repeat sequence may be directly or indirectly 3′ of the spacer sequence.

The system also comprises a template RNA (retRNA). The retRNA comprises a primer binding sequence (PBS) and a template sequence. The template sequence may be an RT template. The retRNA comprises an MS2 localization sequence. The MS2 localization sequence may be located at the 5′ or 3′ terminus of the retRNA. The template sequence is complementary to the target sequence on the non-target strand with the exception of one or more nucleotides conveying an intended edit of the target nucleic acid.

The effector protein is not covalently linked to the RDDP, but the RDDP is linked to an MS2 coat protein that binds the MS2 localization sequence, thereby localizing the RDDP to the target nucleic acid. See, e.g., FIGS. 5A-5B. Alternatively, the guide RNA and retRNA are linked while the effector protein and RDDP are not linked. Also, alternatively, the effector protein and RDDP are linked, while the guide RNA and retRNA are not linked. The effector protein nicks the non-target strand of the target nucleic acid and the RDDP synthesizes new DNA off of the nicked end, wherein the new DNA is complementary to the RT template, thereby producing the desired genomic edit in the target nucleic acid.

### Example 5—Variant RNA-Dependent DNA Polymerases for Precision Editing

Variants of the 2691319 RDDP (SEQ ID NO: 22) and 2691323 RDDP (SEQ ID NO: 24) were generated and tested for their ability to perform precision editing. Combinations of arginine and lysine mutations were made at positions D12, N24, D72, N195, and N114 as follows:
(a) 2691319 (D12R-D72R-N195R)—SEQ ID NO: 80
(b) 2691319 (D12R-N24R-D72R-N114K)—SEQ ID NO: 81
(c) 2691319 (D12R-N24R-D72R-N195R)—SEQ ID NO: 82
(d) 2691319 (D12R-D72R-N114K-N195R)—SEQ ID NO: 83
(e) 2691319 (D12R-N24R-D72R-N114K-N195R)—SEQ ID NO: 84
(f) 2691323 (D12R-N72R-N195R)—SEQ ID NO: 85
(g) 2691323 (D12R-N24R-N72R-N114K)—SEQ ID NO: 86
(h) 2691323 (D12R-N24R-N72R-N195R)—SEQ ID NO: 87
(i) 2691323 (D12R-N72R-N114K-N195R)—SEQ ID NO: 88
(j) 2691323 (D12R-N24R-N72R-N114K-N195R)—SEQ ID NO: 89.

Each of the engineered RDDP candidates was linked to nCas9 (H840A) (SEQ ID NO: 1594) on the N terminus or the C terminus, separated by an XTEN40 linker (SEQ ID NO: 1595).

### Example 6-CasM.265466 and its Engineered Variants Mediate DMD Exon Editing in HEK293T Cells

This example demonstrates the ability of CasM.265466, as set forth in SEQ ID NO: 1, or its engineered variants comprising at least one amino acid alteration provided in TABLE 1.1 in mediating DMD exon editing. The experiment utilizes CasM.265466 or its engineered variants in combination with a gRNA selected from the sequences provided in TABLE 6 and an retRNA selected from the sequences provided in TABLE 7. This experiment employs a split protein design and split RNA design, as shown in FIG. 6.

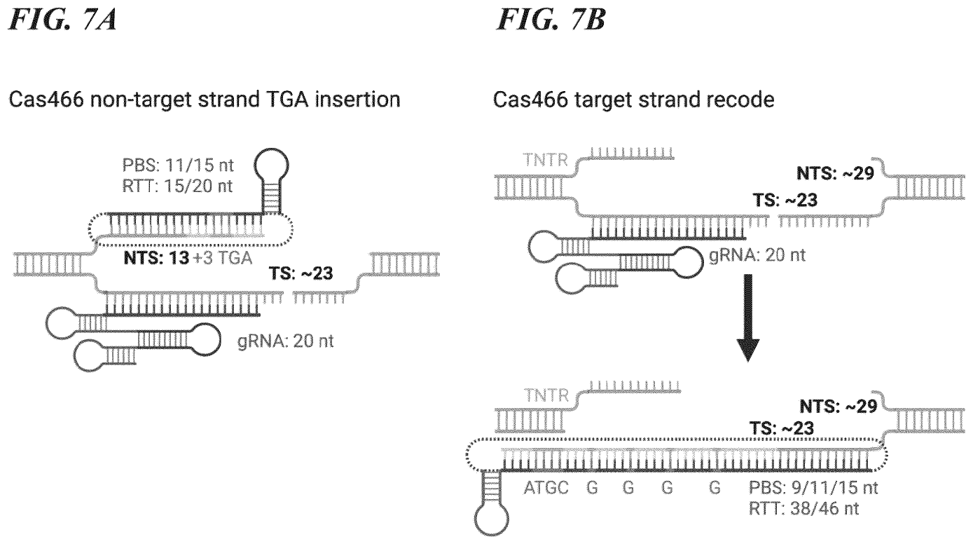

For the non-target strand (NTS), cleavage occurs approximately 29 nt 3′ of the beginning of the spacer (or in other words 29 nt 3′ of the 5′ end of the target sequence on the NTS), then degrades DNA in a 3′->5′ direction until nucleotide 13. The PBS hybridizes to genomic DNA 5′ of nucleotide 13, and a TGA insertion is encoded by the RTT 3 nt after nucleotide 13. Editing of the NTS is represented in FIG. 7A.

For the target strand (TS), cleavage occurs approximately at position 23 relative to the 3′ end of the PAM. Due to the scarcity of TNTR PAMs within the exons, a relaxed NNTR PAM is employed when utilizing CasM.265466 or its engineered variants to target DMD. The PBS hybridizes to NTS 5′ of nucleotide 23, and the precise edit is encoded by the RTT. The precise edit consists of a 4-letter ATGC substitution for the NNTR PAM of CasM.265466 or its engineered variants, along with several single base C substitutions interspersed between the PAM and PBS. Editing of the TS is represented in FIG. 7B.

Briefly, HEK293T are transfected with 300 ng of a first plasmid encoding CasM.265466 or its engineered variant described herein and a full transcribed guide RNA selected from the sequences of SEQ ID NOs: 649-831 and 100 ng of a second plasmid encoding M-MLV reverse transcriptase and a full transcribed retRNA selected from the sequences of SEQ ID NOs: 904-927 using Transit 293T lipofection reagent at a ratio of 3:1 DNA.

Cells are incubated 72 hours and harvested for next generation sequencing (NGS) sequence analysis. Each condition is tested in the presence and absence of NHEJ inhibitor (NHEJi) AZD7648. A retRNA with no sequence similarity to a target site served as a negative control. Indels are detected by NGS at the targeted loci and indel percentage is calculated as the fraction of sequencing reads containing insertions or deletions relative to the unedited D) MI) gene sequence.

The design of CasM.265466 or its engineered variant system targeting Exon 45, 51, and 53 generates a GTAC insertion (+1 nt frameshift) at position 16 relative to the 5′ end of the spacer. The design of CasM.265466 or its engineered variant system targeting Exon 52 generates a GTACC insertion (+2 nt frameshift) at position 16 relative to the 5′ end of the spacer. These designs have the potential to induce frameshift mutations at the target sites, thereby affecting the protein-coding sequence of DMD in the specified exons.

### Example 7-CasPhi.12 and its Engineered Variants Mediate DMD Exon Editing in HEK293T Cells

This example demonstrates the ability of CasPhi. 12 (as set forth in SEQ ID NO: 2) or its engineered variants comprising at least one amino acid alteration provided in TABLE 1.2 in mediating D) MI) exon editing. The experiment utilizes CasPhi.12 or its engineered variants in combination with a gRNA selected from the sequences provided in TABLE 8 and an retRNA selected from the sequences provided in TABLE 9. This experiment employs a split protein design and split RNA design, as shown in FIG. 9.

Briefly, HEK293T are transfected with 300 ng of a first plasmid encoding CasPhi.12 or its engineered variant as described herein and a full transcribed guide RNA selected from the sequences of SEQ ID NOs: 1354-1495 and 100 ng of a second plasmid encoding M-MLV reverse transcriptase and a full transcribed retRNA selected from the sequences of SEQ ID NOs: 1568-1591 using Transit 293T lipofection reagent at a ratio of 3:1 DNA.

Cells are incubated 72 hours and harvested for NGS sequence analysis. Each condition is tested in the presence and absence of NHEJ inhibitor (NHEJi) AZD7648. A retRNA with no sequence similarity to a target site served as a negative control. Indels are detected by NGS at the targeted loci and indel percentage is calculated as the fraction of sequencing reads containing insertions or deletions relative to the unedited DMD gene sequence.

The design of CasPhi.12 or its engineered variant system targeting Exon 45, 51, and 53 generates a GTAC insertion (+1 nt frameshift) at position 17 relative to the 5′ end of the spacer. The design of CasPhi.12 or its engineered variant system targeting Exon 52 generates a GTACC insertion (+2 nt frameshift) at position 17 relative to the 5′ end of the spacer. These designs have the potential to induce frameshift mutations at the target sites, thereby affecting the protein-coding sequence of DMD in the specified exons.

### Example 8-In Vivo Muscle Targeting Via AAV9-4A Delivery of CasPhi.12 and CasM.265466 Variants

The purpose of the study was to assess the capability of two effector protein variants, CasPhi.12 L26R (a variant of CasPhi.12) and CasM.265466 D220R (a variant of CasM.265466), to edit nucleic acid sequences within muscle tissues in vivo. The study focused on PCSK9 as an exemplary gene target.

In this study, an AAV9-4A vector was employed as the delivery vehicle for introducing the effector protein and gRNA into the specific target tissues. The DNA encoding the effector protein (e.g., SaCas9, CasPhi.12 L26R, or CasM.265466 D220R) and its corresponding promoter (e.g., ck8e or spc5), along with the DNA encoding the gRNA containing the targeting spacer sequence specific to PCSK9 (referred to as PCSK9 in the plasmid) and its u6 promoter were cloned into the AAV9-4A plasmid between the AAV inverted terminal repeats (ITRs), creating AAV9 constructs as follows:
1) pssAAV-ITR-u6-PCSK9-ck8e-saCas9-bGHpolya-ITR (PL26295)
2) pssAAV-ITR-u6-PCSK9-ck8e-L26R-wpre-hGHpolya-ITR (PL26297)
3) pssAAV-ITR-u6-PCSK9-spc5-12-L26R-wpre-hGHpolya-ITR (PL31718)
4) pssAAV-ITR-u6-PCSK9-ck8e-D220R-wpre-hGHpolya-ITR (PL31719)
5) pssAAV-ITR-u6-PCSK9-spc5-12-D220R-wpre-hGHpolya-ITR (PL31720)
6) pscAAV-ITR-u6-PCSK9-Spc5-12-D220R-HSVpolyA-ITR (PL31721)

The sequences of the gRNA designed for use in conjunction with SaCas9, CasPhi.12 L26R, or CasM.265466 D220R for targeting the mouse PCSK9 gene are provided in TABLE 14.

TABLE 14
Exemplary gRNA sequences

| Target | | | PAM |
|---|---|---|---|
| Locus | Nuclease | Spacer sequence | sequence |
| PCSK9 | SaCas9 | CACCGCAGCCAC | NNGRRT |
| | | GCAGAGCA | (GTGGGT) |
| | | (SEQ ID NO: 1596) | |
| PCSK9 | CasPhi.12 | GAGCAACGGCGG | TTN |
| | L26R | AAGGU | (TTG) |
| | | (SEQ ID NO: 1597) | |
| PCSK9 | CasM.265466 | UAGAACCUUGAUGACAUAGC | TNTR |
| | D220R | (SEQ ID NO: 1598) | (TATG) |

The gRNA for CasPhi.12 comprises a repeat
sequence of AUUGCUCCUUACGAGGAGAC
(SEQ ID NO: 6)
The gRNA for CasM.265466 D220R comprises a
handle sequence of ACAGCUUAUUUGGAAGCUGAAAU
GUGAGGUUUAUAACACUCACAAGAAUCCUGAAAA
AGGAUGCCAAAC (SEQ ID NO: 4)

The sequences of the other AAV components are provided in TABLE 15.

TABLE 15
The sequences of AAV components

| AAV | | SEQ ID |
|---|---|---|
| Compo- | | |
| nents | Sequences | NO: |
| Ck8e | cgctccggtgcccgtTGCCCATGTAAGGAG | 1599 |
| promoter | GCAAGGCCTGGGGACACCCGAGATGCCTGG | |
| | TTATAATTAACCCAGACATGTGGCTGCCCC | |
| | CCCCCCCCCAACACCTGCTGCCTCTAAAAA | |
| | TAACCCTGCATGCCATGTTCCCGGCGAAGG | |
| | GCCAGCTGTCCCCCGCCAGCTAGACTCAGC | |
| | ACTTAGTTTAGGAACCAGTGAGCAAGTCAG | |
| | CCCTTGGGGCAGCCCATACAAGGCCATGGG | |
| | GCTGGGCAAGCTGCACGCCTGGGTCCGGGG | |
| | TGGGCACGGTGCCCGGGCAACGAGCTGAAA | |
| | GCTCATCTGCTCTCAGGGGCCCCTCCCTGG | |
| | GGACAGCCCCTCCTGGCTAGTCACACCCTG | |
| | TAGGCTCCTCTATATAACCCAGGGGCACAG | |
| | GGGCTGCCCTCATTCTACCACCACCTCCAC | |
| | AGCACAGACAGACACTCAGGAGCCAGCCAG | |
| | Ca | |
| Spc5-12 | ggtaccgctccggtgcccgtCGAGCTCCAC | 1600 |
| promoter | CGCGGTGGCGGCCGTCCGCCCTCGGCACCA | |
| | TCCTCACGACACCCAAATATGGCGACGGGT | |
| | GAGGAATGGTGGGGAGTTATTTTTAGAGCG | |
| | GTGAGGAAGGTGGGCAGGCAGCAGGTGTTG | |
| | GCGCTCTAAAAATAACTCCCGGGAGTTATT | |
| | TTTAGAGCGGAGGAATGGTGGACACCCAAA | |
| | TATGGCGACGGTTCCTCACCCGTCGCCATA | |
| | TTTGGGTGTCCGCCCTCGGCCGGGGCCGCA | |
| | TTCCTGGGGGCCGGGCGGTGCTCCCGCCCG | |
| | CCTCGATAAAAGGCTCCGGGGCCGGCGGCG | |
| | GCCCACGAGCTACCCGGAGGAGCGGGAGGC | |
| | GCCAAGCTCTAGAACTAGTGGATCCCCCGG | |
| | GCTGCAGGAATTCaccggt | |
| U6 | GAGGGCCTATTTCCCATGATTCCTTCATAT | 1601 |
| promoter | TTGCATATACGATACAAGGCTGTTAGAGAG | |
| | ATAATTGGAATTAATTTGACTGTAAACACA | |
| | AAGATATTAGTACAAAATACGTGACGTAGA | |
| | AAGTAATAATTTCTTGGGTAGTTTGCAGTT | |
| | TTAAAATTATGTTTTAAAATGGACTATCAT | |
| | ATGCTTACCGTAACTTGAAAGTATTTCGAT | |
| | TTCTTGGCTTTATATATCTTGTGGAAAGGA | |
| | CGAAACACC | |
| CasM. | MSVLTRKVQLIPVGDKEERDRVYKYLRDGI | 1602 |
| 265466 | EAQNRAMNLYMSGLYFAAINEASKEDRKEL | |
| D220R | NQLYSRIATSSKGSAYTTDIEFPTGLASTS | |
| | TLSMAVRQDFTKSLKDGLMYGRVSLPTYRK | |
| | DNPLFVDVRFVALRGTKQKYNGLYHEYKSH | |
| | TEFLDNLYSSDLKVYIKFANDITFQVIFGN | |
| | PRKSSALRSEFQNIFEEYYKVCQSSIQFSG | |
| | TKIILNMAMRIPDKEIELDEDVCVGVDLGI | |
| | AIPAVCALNKNRYSRVSIGSKEDFLRVRTK | |
| | IRNQRKRLQTNLKSSNGGHGRKKKMKPMDR | |
| | FRDYEANWVQNYNHYVSRQVVDFAVKNKAK | |
| | YINLENLEGIRDDVKNEWLLSNWSYYQLQQ | |
| | YITYKAKTYGIEVRKINPYHTSQRCSCCGY | |
| | EDAGNRPKKEKGQAYFKCLKCGEEMNADFN | |
| | AARNIAMSTEFQSGKKTKKQKKEQHENK | |
| CasPhi. | MIKPTVSQFLTPGFKLIRNHSRTAGRKLKN | 1603 |
| 12L26R | EGEEACKKFVRENEIPKDECPNFQGGPAIA | |
| | NIIAKSREFTEWEIYQSSLAIQEVIFTLPK | |
| | DKLPEPILKEEWRAQWLSEHGLDTVPYKEA | |
| | AGLNLIIKNAVNTYKGVQVKVDNKNKNNLA | |
| | KINRKNEIAKLNGEQEISFEEIKAFDDKGY | |
| | LLQKPSPNKSIYCYQSVSPKPFITSKYHNV | |
| | NLPEEYIGYYRKSNEPIVSPYQFDRLRIPI | |
| | GEPGYVPKWQYTFLSKKENKRRKLSKRIKN | |
| | VSPILGIICIKKDWCVFDMRGLLRTNHWKK | |
| | YHKPTDSINDLFDYFTGDPVIDTKANVVRF | |
| | RYKMENGIVNYKPVREKKGKELLENICDQN | |
| | GSCKLATVDVGQNNPVAIGLFELKKVNGEL | |
| | TKTLISRHPTPIDFCNKITAYRERYDKLES | |
| | SIKLDAIKQLTSEQKIEVDNYNNNFTPQNT | |
| | KQIVCSKLNINPNDLPWDKMISGTHFISEK | |
| | AQVSNKSEIYFTSTDKGKTKDVMKSDYKWF | |
| | QDYKPKLSKEVRDALSDIEWRLRRESLEFN | |
| | KLSKSREQDARQLANWISSMCDVIGIENLV | |
| | KKNNFFGGSGKREPGWDNFYKPKKENRWWI | |
| | NAIHKALTELSQNKGKRVILLPAMRTSITC | |
| | PKCKYCDSKNRNGEKFNCLKCGIELNADID | |
| | VATENLATVAITAQSMPKPTCERSGDAKKP | |
| | VRARKAKAPEFHDKLAPSYTVVLREAV | |
| WPRE | AATCAACCTCTGGATTACAAAATTTGTGAA | 1604 |
| | AGATTGACTGGTATTCTTAACTATGTTGCT | |
| | CCTTTTACGCTATGTGGATACGCTGCTTTA | |
| | ATGCCTTTGTATCATGCTATTGCTTCCCGT | |
| | ATGGCTTTCATTTTCTCCTCCTTGTATAAA | |
| | TCCTGGTTGCTGTCTCTTTATGAGGAGTTG | |
| | TGGCCCGTTGTCAGGCAACGTGGCGTGGTG | |
| | TGCACTGTGTTTGCTGACGCAACCCCCACT | |
| | GGTTGGGGCATTGCCACCACCTGTCAGCTC | |
| | CTTTCCGGGACTTTCGCTTTCCCCCTCCCT | |
| | ATTGCCACGGCGGAACTCATCGCCGCCTGC | |
| | CTTGCCCGCTGCTGGACAGGGGCTCGGCTG | |
| | TTGGGCACTGACAATTCCGTGGTGTTGTCG | |
| | GGGAAGCTGACGTCCTTTCCATGGCTGCTC | |
| | GCCTGTGTTGCCACCTGGATTCTGCGCGGG | |
| | ACGTCCTTCTGCTACGTCCCTTCGGCCCTC | |
| | AATCCAGCGGACCTTCCTTCCCGCGGCCTG | |
| | CTGCCGGCTCTGCGGCCTCTTCCGCGTCTT | |
| | gGCCTTCGCCCTCAGACGAGTCGGATCTCC | |
| | CTTTGGGCCGCCTCCCCGC | |
| bGH | CTGTGCCTTCTAGTTGCCAGCCATCTGTTG | 1605 |
| PolyA | TTTGCCCCTCCCCCGTGCCTTCCTTGACCC | |
| | TGGAAGGTGCCACTCCCACTGTCCTTTCCT | |
| | AATAAAATGAGGAAATTGCATCGCATTGTC | |
| | TGAGTAGGTGTCATTCTATTCTGGGGGGTG | |
| | GGGTGGGGCAGGACAGCAAGGGGGAGGATT | |
| | GGGAAGAGAATAGCAGGCATGCTGGGGA | |
| hGH | ACGGGTGGCATCCCTGTGACCCCTCCCCAG | 1606 |
| PolyA | TGCCTCTCCTGGCCCTGGAAGTTGCCACTC | |
| | CAGTGCCCACCAGCCTTGTCCTAATAAAAT | |
| | TAAGTTGCATCATTTTGTCTGACTAGGTGT | |
| | CCTTCTATAATATTATGGGGTGGAGGGGGG | |
| | TGGTATGGAGCAAGGGGCAAGTTGGGAACA | |
| | CAACCTGTAGGGCCTGCGGGGTCTATTGGG | |
| | AACCAAGCTGGAGTGCAGTGGCACAATCTT | |
| | GGCTCACTGCAATCTCCGCCTCCTGGGTTC | |
| | AAGCGATTCTCCTGCCTCAGCCTCCCGAGT | |
| | TGTTGGGATTCCAGGCATGCATGACCAGGC | |
| | TCAGCTAATTTTTGTTTTTTTGGTAGAGAC | |
| | GGGGTTTCACCATATTGGCCAGGCTGGTCT | |
| | CCAACTCCTAATCTCAGGTGATCTACCCAC | |
| | CTTGGCCTCCCAAATTGCTGGGATTACAGG | |
| | CGTGAACCACTGCTCCCTTCCCTGTCCTTC | |
| | TGATTTTGTAGGTAACCACGTGCGGACCGA | |

6-week old male C57BL/6J mice were given a single intravenous (IV) bolus through the tail vein using a 1.0 cc syringe with a 27G-⅝″ needle or a single intramuscular (IM) bolus into the gastrocnemius. The study design is provided in TABLE 16.

TALBE 16
Study design

| | | Size | | |
|---|---|---|---|---|
| Groups | Construct design (PL#) | (Kb) | Route, dose | N |
| 1 | Vehicle (PBS) | — | — | 15 |
| 2, 3 | pssAAV-ITR-u6-PCSK9-ck8e-saCas9- | 4.7 | 14 vg/kg IV and 12 vg | 20 |
| | hGHpolya-ITR (PL26295) | | IM | |
| 4, 5 | pssAAV-ITR-u6-PCSK9-ck8e-L26R- | 4.6 | 14 vg/kg IV and 12 vg | 20 |
| | wpre-hGHpolya-ITR (PL26297) | | IM | |
| 6 | pssAAV-ITR-u6-PCSK9-spc5-12-L26R- | 4.5 | 14 vg/kg IV | 10 |
| | wpre-hGHpolya-ITR (PL31718) | | | |
| 7 | pssAAV-ITR-u6-PCSK9-ck8e-D220R- | 3.8 | 14 vg/kg IV | 10 |
| | wpre-hGHpolya-ITR (PL31719) | | | |
| 8 | pssAAV-ITR-u6-PCSK9-spc5-12- | 3.79 | 14 vg/kg IV | 10 |
| | D220R-wpre-hGHpolya-ITR (PL31720) | | | |
| 9 | pssAAV-ITR-U6-PCSK9-Spc5-12- | 2.48 | 14 vg/kg IV | 15 |
| | D220R-HSVpolyA-ITR (PL31721) | | | |

4 weeks after the treatment, the mice were euthanized for assessment. Before collecting the tissues, the mice underwent whole-body perfusion via the left ventricle using 5.0-10.0 mL of PBS to remove any remaining blood from the tissues. Tissue samples weighing 100 mg were then dissected from the liver, heart, gastrocnemius, diaphragm, pectoral, and masseter, respectively, and placed on a plate for subsequent Next-Generation Sequencing (NGS) analysis. For the intramuscular (IM) groups, both the left and right gastrocnemius were weighed and harvested. For all other groups, only the left gastrocnemius was weighed and harvested.

The genomic DNA isolated from the muscle tissues was subject to NGS and aligned to a reference DNA sequence for the analysis of insertions or deletions (indels).

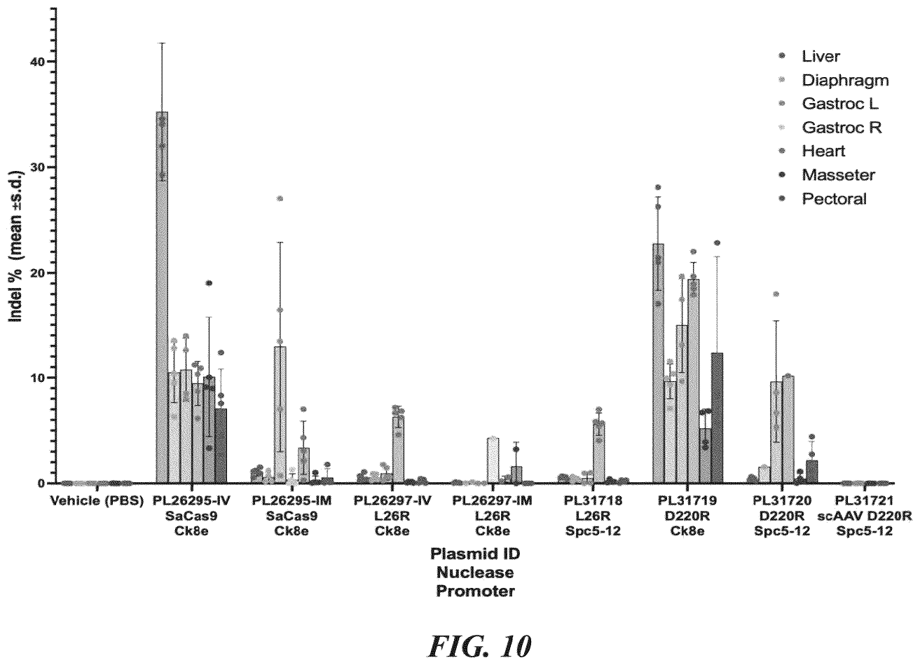

In FIG. 10, the data reveals that CasPhi.12 L26R, delivered in the PL26297 vector via IV administration, resulted in an about 8% indel rate in the PCSK9 gene in the heart. Likewise, CasPhi. 12 L26R delivered in the PL26297 vector via IM administration generated about 5% indel rate in the right gastrocnemius and about 3% indel rate in masseter. Additionally, CasPhi.12 L26R delivered in the PL31718 vector via IV administration was able to generate about 8% indel rate in the PCSK9 gene in the heart.

As shown in FIG. 10, CasM.265466 D220R delivered in the PL31719 vector via IV administration resulted in an about 25% indel rate in the liver, about 10% in the diaphragm, about 15% in the left gastrocnemius, about 20% in the heart, about 5% in the masseter, and about 13% in the pectoral. Remarkably, CasM.265466 D220R demonstrated an approximately 2-fold greater indel rate in the heart compared to SaCas9 delivered in the PL26295 vector via IV administration. Additionally, CasM.265466 D220R delivered in the PL31719 vector via IV administration generated about 10% in the left gastrocnemius, about 11% in the heart, and about 3% in the pectoral.

The results demonstrate that both CasPhi.12 variant L26R and CasM.265466 variant D220R are highly efficient in inducing indels in vivo at the PCSK9 locus across different muscle tissues.

The in vivo experiment will be repeated using the gRNA for targeting DMD in conjunction with any one of the Cas variants disclosed herein.

### Example 9—CasPhi.12 Engineered Variant Mediate DMD Exon Editing in HEK293T Cells

This example demonstrated the ability of CasPhi.12 engineered variant comprising amino acid substitutions L26R, 1471T, S223P, and D703G, as set forth in SEQ ID NO: 1607, in mediating DMD exon editing.

HEK293T cells were transfected with plasmids encoding effector protein CasPhi.12 L26R, I471T, S223P, D703G (SEQ ID NO: 1607), a reverse transcriptase (RT) (SEQ ID NO: 1608), a retRNA (SEQ ID NO: 1590), and a guide nucleic acid targeting DMD (SEQ ID NO: 1327). The effector protein was not linked to the RT, but the RT was fused to an MS2 coat protein (MCP) capable of binding an MS2 aptamer loop in the retRNA. Exemplary RT and retRNA sequences are provided in TABLE 17. Cells were harvested 72 hours later and cell lysates were prepared for Amplicon Sequencing. The percentage of reads containing precise edits were analyzed. An average of 1.6% precise editing was achieved with this system, one replicate achieving 2.1% precise editing.

TABLE 17
Exemplary RT and retRNA sequences

| | | SEQ ID |
|---|---|---|
| | Sequence | NO: |
| MCP- | MASNFTQFVLVDNGGTGDVTVAPSNFANGV | 1608 |
| MMLV | AEWISSNSRSQAYKVTCSVRQSSAQKRKY | |
| | TIKVEVPKVATQTVGGVELPVAAWRSYLN | |
| | MELTIPIFATNSDCELIVKAMQGLLKDGN | |
| | PIPSAIAANSGIYSAGGGGSGGGGSGGGG | |
| | SGPKKKRKVAAAGSTLNIEDEYRLHETSK | |
| | EPDVSLGSTWLSDFPQAWAETGGMGLAVR | |
| | QAPLIIPLKATSTPVSIKQYPMSQEARLG | |
| | IKPHIQRLLDQGILVPCQSPWNTPLLPVK | |
| | KPGTNDYRPVQDLREVNKRVEDIHPTVPN | |
| | PYNLLSGLPPSHQWYTVLDLKDAFFCLRL | |
| | HPTSQPLFAFEWRDPEMGISGQLTWTRLP | |
| | QGFKNSPTLFNEALHRDLADFRIQHPDLI | |
| | LLQYVDDLLLAATSELDCQQGTRALLQTL | |
| | GNLGYRASAKKAQICQKQVKYLGYLLKEG | |
| | QRWLTEARKETVMGQPTPKTPRQLREFLG | |
| | KAGFCRLFIPGFAEMAAPLYPLTKPGTLF | |
| | NWGPDQQKAYQEIKQALLTAPALGLPDLT | |
| | KPFELFVDEKQGYAKGVLTQKLGPWRRPV | |
| | AYLSKKLDPVAAGWPPCLRMVAAIAVLTK | |
| | DAGKLTMGQPLVILAPHAVEALVKQPPDR | |
| | WLSNARMTHYQALLLDTDRVQFGPVVALN | |
| | PATLLPLPEEGLQHNCLDILAEAHGTRPD | |
| | LTDQPLPDADHTWYTDGSSLLQEGQRKAG | |
| | AAVTTETEVIWAKALPAGTSAQRAELIAL | |
| | TQALKMAEGKKLNVYTDSRYAFATAHIHG | |
| | EIYRRRGWLTSEGKEIKNKDEILALLKAL | |
| | FLPKRLSIIHCPGHQKGHSAEARGNRMAD | |
| | QAARKAAITETPDTSTLLIENSSP | |
| | (Italic: MS2 coat protein (MCP); | |
| | underline: NLS; | |
| | bold: engineered MMLV | |
| | (SEQ ID NO: 1593)) | |
| retRNA | GUGCUCGCUUCGGCAGCACAUAUACUAG | 1590 |
| | UCGACGGGCCGCACUCGCCGGUCCCAAG | |
| | CCCGGAUAAAAUGGGAGGGGGCGGGAAA | |
| | CCGCCUAACCAUGCCGAGUGCGGCCGCA | |
| | ACUAAGCACAUGAGGAUCACCCAUGUGC | |
| | UACAGGAACUCCAGGAGUACUGGCAUUG | |
| | GGCAGCGAAAUUAACAGUGGCCGCGGUC | |
| | GGCGUGGACUGUAGAACACUGCCAAUGC | |
| | CGGUCCCAAGCCCGGAUAAAAGUGGAGG | |
| | GUACAGUCCACGCUCUAGAGCGGACUUC | |
| | GGUCCGC | |
| | (Italic: Ribozyme; | |
| | underline: MS2 loop; | |
| | bold: RTT/PBS (SEQ ID NO: 1566)) | |

### Example 10. Dual-Cut Dual-Flap for Precise Deletions of a Region of DMD

Various systems, referred to as dual-cut dual-flap systems, for precise deletions were tested in this experiment. A non-limiting representative illustration of a dual-cut, dual-flap system is provided in FIG. 11. These systems comprised two guide RNAs targeting DMD and two RT template RNAs (retRNA) to create precise deletions. The effector protein used in these systems was CasM.265466 with a D220R amino acid substitution, denoted in this example as 466 (D220R). Various plasmids encoding the fusion proteins in TABLE 18 below were tested. Note however that the presence of a P2A peptide results in cleavage of the fusion protein at the site of the P2A peptide. Some of the protein fusions also have a Brex27 peptide fused that inhibits NHEJ. The amino acid sequence of the Brex27 peptide was set forth in SEQ ID NO: 1625 and the amino acid sequence of the MCP (MS2 aptamer binding protein) was set forth in SEQ ID NO: 1626. These systems were screened with and without a small peptide NHEJ inhibitor (UbvG08). UbvG08 was expressed separately and not fused to the effector protein or the RT. Guide RNA sequences targeting DMD and retRNA sequences used in this experiment are provided in TABLE 19. Results are provided in TABLE 20. Briefly, HEK293T cells were transfected with plasmids encoding the various proteins and guide RNAs. The retRNAs encoded a −38 precise deletion. Precise edits were quantified using amplicon NGS. Blasticidin selection was applied 24 hours after transfection.

TABLE 18
Description of Fusion Proteins

| Protein | | |
|---|---|---|
| Fusion Protein | | SEQ |
| Description | Amino Acid Sequence | ID |
| Brex27- | AATMKRTADGSEFESPKKKRKVALDFLSRLPLPPPVSPICTFVSP | 1610 |
| 466(D220R)-MCP- | AAQKAFQPPRSCGSATPGSSRMSVLTRKVQLIPVGDKEERDRVYK | |
| MLV---TS long | YLRDGIEAQNRAMNLYMSGLYFAAINEASKEDRKELNQLYSRIAT | |
| combined* | SSKGSAYTTDIEFPTGLASTSTLSMAVRQDFTKSLKDGLMYGRVS | |
| | LPTYRKDNPLFVDVRFVALRGTKQKYNGLYHEYKSHTEFLDNLYS | |
| | SDLKVYIKFANDITFQVIFGNPRKSSALRSEFQNIFEEYYKVCQS | |
| | SIQFSGTKIILNMAMRIPDKEIELDEDVCVGVDLGIAIPAVCALN | |
| | KNRYSRVSIGSKEDFLRVRTKIRNQRKRLQTNLKSSNGGHGRKKK | |
| | MKPMDRFRDYEANWVQNYNHYVSRQVVDFAVKNKAKYINLENLEG | |
| | IRDDVKNEWLLSNWSYYQLQQYITYKAKTYGIEVRKINPYHTSQR | |
| | CSCCGYEDAGNRPKKEKGQAYFKCLKCGEEMNADFNAARNIAMST | |
| | EFQSGKKTKKQKKEQHENKKRPAATKKAGQAKKKKSRKRTADGSE | |
| | FESPKKKRKVASNFTQFVLVDNGGTGDVTVAPSNFANGVAEWISS | |
| | NSRSQAYKVTCSVRQSSAQKRKYTIKVEVPKVATQTVGGVELPVA | |
| | AWRSYLNMELTIPIFATNSDCELIVKAMQGLLKDGNPIPSAIAAN | |
| | SGIYSGGSSGGSTLNIEDEYRLHETSKEPDVSLGSTWLSDFPQAW | |
| | AETGGMGLAVRQAPLIIPLKATSTPVSIKQYPMSQEARLGIKPHI | |
| | QRLLDQGILVPCQSPWNTPLLPVKKPGTNDYRPVQDLREVNKRVE | |
| | DIHPTVPNPYNLLSGLPPSHQWYTVLDLKDAFFCLRLHPTSQPLF | |
| | AFEWRDPEMGISGQLTWTRLPQGFKNSPTLFNEALHRDLADFRIQ | |
| | HPDLILLQYVDDLLLAATSELDCQQGTRALLQTLGNLGYRASAKK | |
| | AQICQKQVKYLGYLLKEGQRWLTEARKETVMGQPTPKTPRQLREF | |
| | LGKAGFCRLFIPGFAEMAAPLYPLTKPGTLFNWGPDQQKAYQEIK | |
| | QALLTAPALGLPDLTKPFELFVDEKQGYAKGVLTQKLGPWRRPVA | |
| | YLSKKLDPVAAGWPPCLRMVAAIAVLTKDAGKLTMGQPLVILAPH | |
| | AVEALVKQPPDRWLSNARMTHYQALLLDTDRVQFGPVVALNPATL | |
| | LPLPEEGLQHNCLDILAEAHGTRPDLTDQPLPDADHTWYTDGSSL | |
| | LQEGQRKAGAAVTTETEVIWAKALPAGTSAQRAELIALTQALKMA | |
| | EGKKLNVYTDSRYAFATAHIHGEIYRRRGWLTSEGKEIKNKDEIL | |
| | ALLKALFLPKRLSIIHCPGHQKGHSAEARGNRMADQAARKAAITE | |
| | TPDTSTLLIENSSPSGGSKRTADGSEFESPKKKRKVGSGPAAKRV | |
| | KLDGSG | |
| Brex27- | AATMKRTADGSEFESPKKKRKVALDFLSRLPLPPPVSPICTFVSP | 1611 |
| 466(D220R)-P2A- | AAQKAFQPPRSCGSATPGSSRMSVLTRKVQLIPVGDKEERDRVYK | |
| MCP-MMLV---TS | YLRDGIEAQNRAMNLYMSGLYFAAINEASKEDRKELNQLYSRIAT | |
| long combined | SSKGSAYTTDIEFPTGLASTSTLSMAVRQDFTKSLKDGLMYGRVS | |
| | LPTYRKDNPLFVDVRFVALRGTKQKYNGLYHEYKSHTEFLDNLYS | |
| | SDLKVYIKFANDITFQVIFGNPRKSSALRSEFQNIFEEYYKVCQS | |
| | SIQFSGTKIILNMAMRIPDKEIELDEDVCVGVDLGIAIPAVCALN | |
| | KNRYSRVSIGSKEDFLRVRTKIRNQRKRLQTNLKSSNGGHGRKKK | |
| | MKPMDRFRDYEANWVQNYNHYVSRQVVDFAVKNKAKYINLENLEG | |
| | IRDDVKNEWLLSNWSYYQLQQYITYKAKTYGIEVRKINPYHTSQR | |
| | CSCCGYEDAGNRPKKEKGQAYFKCLKCGEEMNADFNAARNIAMST | |
| | EFQSGKKTKKQKKEQHENKKRPAATKKAGQAKKKKSRATNFSLLK | |
| | QAGDVEENPGPKRTADGSEFESPKKKRKVASNFTQFVLVDNGGTG | |
| | DVTVAPSNFANGVAEWISSNSRSQAYKVTCSVRQSSAQKRKYTIK | |
| | VEVPKVATQTVGGVELPVAAWRSYLNMELTIPIFATNSDCELIVK | |
| | AMQGLLKDGNPIPSAIAANSGIYSGGSSGGSTLNIEDEYRLHETS | |
| | KEPDVSLGSTWLSDFPQAWAETGGMGLAVRQAPLIIPLKATSTPV | |
| | SIKQYPMSQEARLGIKPHIQRLLDQGILVPCQSPWNTPLLPVKKP | |
| | GTNDYRPVQDLREVNKRVEDIHPTVPNPYNLLSGLPPSHQWYTVL | |
| | DLKDAFFCLRLHPTSQPLFAFEWRDPEMGISGQLTWTRLPQGFKN | |
| | SPTLFNEALHRDLADFRIQHPDLILLQYVDDLLLAATSELDCQQG | |
| | TRALLQTLGNLGYRASAKKAQICQKQVKYLGYLLKEGQRWLTEAR | |
| | KETVMGQPTPKTPRQLREFLGKAGFCRLFIPGFAEMAAPLYPLTK | |
| | PGTLFNWGPDQQKAYQEIKQALLTAPALGLPDLTKPFELFVDEKQ | |
| | GYAKGVLTQKLGPWRRPVAYLSKKLDPVAAGWPPCLRMVAAIAVL | |
| | TKDAGKLTMGQPLVILAPHAVEALVKQPPDRWLSNARMTHYQALL | |
| | LDTDRVQFGPVVALNPATLLPLPEEGLQHNCLDILAEAHGTRPDL | |
| | TDQPLPDADHTWYTDGSSLLQEGQRKAGAAVTTETEVIWAKALPA | |
| | GTSAQRAELIALTQALKMAEGKKLNVYTDSRYAFATAHIHGEIYR | |
| | RRGWLTSEGKEIKNKDEILALLKALFLPKRLSIIHCPGHQKGHSA | |
| | EARGNRMADQAARKAAITETPDTSTLLIENSSPSGGSKRTADGSE | |
| | FESPKKKRKVGSGPAAKRVKLDGSG | |
| Brex27-xten20- | AATMKRTADGSEFESPKKKRKVALDFLSRLPLPPPVSPICTFVSP | 1612 |
| 466(D220R)-MCP- | AAQKAFQPPRSCGSATPGSSRMSVLTRKVQLIPVGDKEERDRVYK | |
| MMLV---TS long | YLRDGIEAQNRAMNLYMSGLYFAAINEASKEDRKELNQLYSRIAT | |
| combined | SSKGSAYTTDIEFPTGLASTSTLSMAVRQDFTKSLKDGLMYGRVS | |
| | LPTYRKDNPLFVDVRFVALRGTKQKYNGLYHEYKSHTEFLDNLYS | |
| | SDLKVYIKFANDITFQVIFGNPRKSSALRSEFQNIFEEYYKVCQS | |
| | SIQFSGTKIILNMAMRIPDKEIELDEDVCVGVDLGIAIPAVCALN | |
| | KNRYSRVSIGSKEDFLRVRTKIRNQRKRLQTNLKSSNGGHGRKKK | |
| | MKPMDRFRDYEANWVQNYNHYVSRQVVDFAVKNKAKYINLENLEG | |
| | IRDDVKNEWLLSNWSYYQLQQYITYKAKTYGIEVRKINPYHTSQR | |
| | CSCCGYEDAGNRPKKEKGQAYFKCLKCGEEMNADFNAARNIAMST | |
| | EFQSGKKTKKQKKEQHENKKRPAATKKAGQAKKKKSRKRTADGSE | |
| | FESPKKKRKVASNFTQFVLVDNGGTGDVTVAPSNFANGVAEWISS | |
| | NSRSQAYKVTCSVRQSSAQKRKYTIKVEVPKVATQTVGGVELPVA | |
| | AWRSYLNMELTIPIFATNSDCELIVKAMQGLLKDGNPIPSAIAAN | |
| | SGIYSGGSSGGSTLNIEDEYRLHETSKEPDVSLGSTWLSDFPQAW | |
| | AETGGMGLAVRQAPLIIPLKATSTPVSIKQYPMSQEARLGIKPHI | |
| | QRLLDQGILVPCQSPWNTPLLPVKKPGTNDYRPVQDLREVNKRVE | |
| | DIHPTVPNPYNLLSGLPPSHQWYTVLDLKDAFFCLRLHPTSQPLF | |
| | AFEWRDPEMGISGQLTWTRLPQGFKNSPTLFNEALHRDLADFRIQ | |
| | HPDLILLQYVDDLLLAATSELDCQQGTRALLQTLGNLGYRASAKK | |
| | AQICQKQVKYLGYLLKEGQRWLTEARKETVMGQPTPKTPRQLREF | |
| | LGKAGFCRLFIPGFAEMAAPLYPLTKPGTLFNWGPDQQKAYQEIK | |
| | QALLTAPALGLPDLTKPFELFVDEKQGYAKGVLTQKLGPWRRPVA | |
| | YLSKKLDPVAAGWPPCLRMVAAIAVLTKDAGKLTMGQPLVILAPH | |
| | AVEALVKQPPDRWLSNARMTHYQALLLDTDRVQFGPVVALNPATL | |
| | LPLPEEGLQHNCLDILAEAHGTRPDLTDQPLPDADHTWYTDGSSL | |
| | LQEGQRKAGAAVTTETEVIWAKALPAGTSAQRAELIALTQALKMA | |
| | EGKKLNVYTDSRYAFATAHIHGEIYRRRGWLTSEGKEIKNKDEIL | |
| | ALLKALFLPKRLSIIHCPGHQKGHSAEARGNRMADQAARKAAITE | |
| | TPDTSTLLIENSSPSGGSKRTADGSEFESPKKKRKVGSGPAAKRV | |
| | KLDGSG | |
| Brex27-xten20- | AATMKRTADGSEFESPKKKRKVALDFLSRLPLPPPVSPICTFVSP | 1613 |
| 466(D220R)-P2A- | AAQKAFQPPRSCGGSGGSPAGSPTSTEEGTSESATPGSGSRMSVL | |
| MCP-MMLV---TS | TRKVQLIPVGDKEERDRVYKYLRDGIEAQNRAMNLYMSGLYFAAI | |
| long combined | NEASKEDRKELNQLYSRIATSSKGSAYTTDIEFPTGLASTSTLSM | |
| | AVRQDFTKSLKDGLMYGRVSLPTYRKDNPLFVDVRFVALRGTKQK | |
| | YNGLYHEYKSHTEFLDNLYSSDLKVYIKFANDITFQVIFGNPRKS | |
| | SALRSEFQNIFEEYYKVCQSSIQFSGTKIILNMAMRIPDKEIELD | |
| | EDVCVGVDLGIAIPAVCALNKNRYSRVSIGSKEDFLRVRTKIRNQ | |
| | RKRLQTNLKSSNGGHGRKKKMKPMDRFRDYEANWVQNYNHYVSRQ | |
| | VVDFAVKNKAKYINLENLEGIRDDVKNEWLLSNWSYYQLQQYITY | |
| | KAKTYGIEVRKINPYHTSQRCSCCGYEDAGNRPKKEKGQAYFKCL | |
| | KCGEEMNADFNAARNIAMSTEFQSGKKTKKQKKEQHENKKRPAAT | |
| | KKAGQAKKKKSRATNFSLLKQAGDVEENPGPKRTADGSEFESPKK | |
| | KRKVASNFTQFVLVDNGGTGDVTVAPSNFANGVAEWISSNSRSQA | |
| | YKVTCSVRQSSAQKRKYTIKVEVPKVATQTVGGVELPVAAWRSYL | |
| | NMELTIPIFATNSDCELIVKAMQGLLKDGNPIPSAIAANSGIYSG | |
| | GSSGGSTLNIEDEYRLHETSKEPDVSLGSTWLSDFPQAWAETGGM | |
| | GLAVRQAPLIIPLKATSTPVSIKQYPMSQEARLGIKPHIQRLLDQ | |
| | GILVPCQSPWNTPLLPVKKPGTNDYRPVQDLREVNKRVEDIHPTV | |
| | PNPYNLLSGLPPSHQWYTVLDLKDAFFCLRLHPTSQPLFAFEWRD | |
| | PEMGISGQLTWTRLPQGFKNSPTLFNEALHRDLADFRIQHPDLIL | |
| | LQYVDDLLLAATSELDCQQGTRALLQTLGNLGYRASAKKAQICQK | |
| | QVKYLGYLLKEGQRWLTEARKETVMGQPTPKTPRQLREFLGKAGF | |
| | CRLFIPGFAEMAAPLYPLTKPGTLFNWGPDQQKAYQEIKQALLTA | |
| | PALGLPDLTKPFELFVDEKQGYAKGVLTQKLGPWRRPVAYLSKKL | |
| | DPVAAGWPPCLRMVAAIAVLTKDAGKLTMGQPLVILAPHAVEALV | |
| | KQPPDRWLSNARMTHYQALLLDTDRVQFGPVVALNPATLLPLPEE | |
| | GLQHNCLDILAEAHGTRPDLTDQPLPDADHTWYTDGSSLLQEGQR | |
| | KAGAAVTTETEVIWAKALPAGTSAQRAELIALTQALKMAEGKKLN | |
| | VYTDSRYAFATAHIHGEIYRRRGWLTSEGKEIKNKDEILALLKAL | |
| | FLPKRLSIIHCPGHQKGHSAEARGNRMADQAARKAAITETPDTST | |
| | LLIENSSPSGGSKRTADGSEFESPKKKRKVGSGPAAKRVKLDGSG | |
| 466(D220R)- | AATMKRTADGSEFESPKKKRKVSRMSVLTRKVQLIPVGDKEERDR | 1614 |
| Brex27-MCP- | VYKYLRDGIEAQNRAMNLYMSGLYFAAINEASKEDRKELNQLYSR | |
| MMLV---TS long | IATSSKGSAYTTDIEFPTGLASTSTLSMAVRQDFTKSLKDGLMYG | |
| combined | RVSLPTYRKDNPLFVDVRFVALRGTKQKYNGLYHEYKSHTEFLDN | |
| | LYSSDLKVYIKFANDITFQVIFGNPRKSSALRSEFQNIFEEYYKV | |
| | CQSSIQFSGTKIILNMAMRIPDKEIELDEDVCVGVDLGIAIPAVC | |
| | ALNKNRYSRVSIGSKEDFLRVRTKIRNQRKRLQTNLKSSNGGHGR | |
| | KKKMKPMDRFRDYEANWVQNYNHYVSRQVVDFAVKNKAKYINLEN | |
| | LEGIRDDVKNEWLLSNWSYYQLQQYITYKAKTYGIEVRKINPYHT | |
| | SQRCSCCGYEDAGNRPKKEKGQAYFKCLKCGEEMNADFNAARNIA | |
| | MSTEFQSGKKTKKQKKEQHENKSATPGSALDFLSRLPLPPPVSPI | |
| | CTFVSPAAQKAFQPPRSCGKRPAATKKAGQAKKKKSRKRTADGSE | |
| | FESPKKKRKVASNFTQFVLVDNGGTGDVTVAPSNFANGVAEWISS | |
| | NSRSQAYKVTCSVRQSSAQKRKYTIKVEVPKVATQTVGGVELPVA | |
| | AWRSYLNMELTIPIFATNSDCELIVKAMQGLLKDGNPIPSAIAAN | |
| | SGIYSGGSSGGSTLNIEDEYRLHETSKEPDVSLGSTWLSDFPQAW | |
| | AETGGMGLAVRQAPLIIPLKATSTPVSIKQYPMSQEARLGIKPHI | |
| | QRLLDQGILVPCQSPWNTPLLPVKKPGTNDYRPVQDLREVNKRVE | |
| | DIHPTVPNPYNLLSGLPPSHQWYTVLDLKDAFFCLRLHPTSQPLF | |
| | AFEWRDPEMGISGQLTWTRLPQGFKNSPTLFNEALHRDLADFRIQ | |
| | HPDLILLQYVDDLLLAATSELDCQQGTRALLQTLGNLGYRASAKK | |
| | AQICQKQVKYLGYLLKEGQRWLTEARKETVMGQPTPKTPRQLREF | |
| | LGKAGFCRLFIPGFAEMAAPLYPLTKPGTLFNWGPDQQKAYQEIK | |
| | QALLTAPALGLPDLTKPFELFVDEKQGYAKGVLTQKLGPWRRPVA | |
| | YLSKKLDPVAAGWPPCLRMVAAIAVLTKDAGKLTMGQPLVILAPH | |
| | AVEALVKQPPDRWLSNARMTHYQALLLDTDRVQFGPVVALNPATL | |
| | LPLPEEGLQHNCLDILAEAHGTRPDLTDQPLPDADHTWYTDGSSL | |
| | LQEGQRKAGAAVTTETEVIWAKALPAGTSAQRAELIALTQALKMA | |
| | EGKKLNVYTDSRYAFATAHIHGEIYRRRGWLTSEGKEIKNKDEIL | |
| | ALLKALFLPKRLSIIHCPGHQKGHSAEARGNRMADQAARKAAITE | |
| | TPDTSTLLIENSSPSGGSKRTADGSEFESPKKKRKVGSGPAAKRV | |
| | KLDGSG | |
| 466(D220R)- | AATMKRTADGSEFESPKKKRKVSRMSVLTRKVQLIPVGDKEERDR | 1615 |
| Brex27-P2A-MCP- | VYKYLRDGIEAQNRAMNLYMSGLYFAAINEASKEDRKELNQLYSR | |
| MMLV---TS long | IATSSKGSAYTTDIEFPTGLASTSTLSMAVRQDFTKSLKDGLMYG | |
| combined | RVSLPTYRKDNPLFVDVRFVALRGTKQKYNGLYHEYKSHTEFLDN | |
| | LYSSDLKVYIKFANDITFQVIFGNPRKSSALRSEFQNIFEEYYKV | |
| | CQSSIQFSGTKIILNMAMRIPDKEIELDEDVCVGVDLGIAIPAVC | |
| | ALNKNRYSRVSIGSKEDFLRVRTKIRNQRKRLQTNLKSSNGGHGR | |
| | KKKMKPMDRFRDYEANWVQNYNHYVSRQVVDFAVKNKAKYINLEN | |
| | LEGIRDDVKNEWLLSNWSYYQLQQYITYKAKTYGIEVRKINPYHT | |
| | SQRCSCCGYEDAGNRPKKEKGQAYFKCLKCGEEMNADFNAARNIA | |
| | MSTEFQSGKKTKKQKKEQHENKSATPGSALDFLSRLPLPPPVSPI | |
| | CTFVSPAAQKAFQPPRSCGKRPAATKKAGQAKKKKSRATNFSLLK | |
| | QAGDVEENPGPKRTADGSEFESPKKKRKVASNFTQFVLVDNGGTG | |
| | DVTVAPSNFANGVAEWISSNSRSQAYKVTCSVRQSSAQKRKYTIK | |
| | VEVPKVATQTVGGVELPVAAWRSYLNMELTIPIFATNSDCELIVK | |
| | AMQGLLKDGNPIPSAIAANSGIYSGGSSGGSTLNIEDEYRLHETS | |
| | KEPDVSLGSTWLSDFPQAWAETGGMGLAVRQAPLIIPLKATSTPV | |
| | SIKQYPMSQEARLGIKPHIQRLLDQGILVPCQSPWNTPLLPVKKP | |
| | GTNDYRPVQDLREVNKRVEDIHPTVPNPYNLLSGLPPSHQWYTVL | |
| | DLKDAFFCLRLHPTSQPLFAFEWRDPEMGISGQLTWTRLPQGFKN | |
| | SPTLFNEALHRDLADFRIQHPDLILLQYVDDLLLAATSELDCQQG | |
| | TRALLQTLGNLGYRASAKKAQICQKQVKYLGYLLKEGQRWLTEAR | |
| | KETVMGQPTPKTPRQLREFLGKAGFCRLFIPGFAEMAAPLYPLTK | |
| | PGTLFNWGPDQQKAYQEIKQALLTAPALGLPDLTKPFELFVDEKQ | |
| | GYAKGVLTQKLGPWRRPVAYLSKKLDPVAAGWPPCLRMVAAIAVL | |
| | TKDAGKLTMGQPLVILAPHAVEALVKQPPDRWLSNARMTHYQALL | |
| | LDTDRVQFGPVVALNPATLLPLPEEGLQHNCLDILAEAHGTRPDL | |
| | TDQPLPDADHTWYTDGSSLLQEGQRKAGAAVTTETEVIWAKALPA | |
| | GTSAQRAELIALTQALKMAEGKKLNVYTDSRYAFATAHIHGEIYR | |
| | RRGWLTSEGKEIKNKDEILALLKALFLPKRLSIIHCPGHQKGHSA | |
| | EARGNRMADQAARKAAITETPDTSTLLIENSSPSGGSKRTADGSE | |
| | FESPKKKRKVGSGPAAKRVKLDGSG | |
| 466(D220R)-MCP- | AATMKRTADGSEFESPKKKRKVSRMSVLTRKVQLIPVGDKEERDR | 1616 |
| MMLV---TS long | VYKYLRDGIEAQNRAMNLYMSGLYFAAINEASKEDRKELNQLYSR | |
| combined | IATSSKGSAYTTDIEFPTGLASTSTLSMAVRQDFTKSLKDGLMYG | |
| | RVSLPTYRKDNPLFVDVRFVALRGTKQKYNGLYHEYKSHTEFLDN | |
| | LYSSDLKVYIKFANDITFQVIFGNPRKSSALRSEFQNIFEEYYKV | |
| | CQSSIQFSGTKIILNMAMRIPDKEIELDEDVCVGVDLGIAIPAVC | |
| | ALNKNRYSRVSIGSKEDFLRVRTKIRNQRKRLQTNLKSSNGGHGR | |
| | KKKMKPMDRFRDYEANWVQNYNHYVSRQVVDFAVKNKAKYINLEN | |
| | LEGIRDDVKNEWLLSNWSYYQLQQYITYKAKTYGIEVRKINPYHT | |
| | SQRCSCCGYEDAGNRPKKEKGQAYFKCLKCGEEMNADFNAARNIA | |
| | MSTEFQSGKKTKKQKKEQHENKKRPAATKKAGQAKKKKSRKRTAD | |
| | GSEFESPKKKRKVASNFTQFVLVDNGGTGDVTVAPSNFANGVAEW | |
| | ISSNSRSQAYKVTCSVRQSSAQKRKYTIKVEVPKVATQTVGGVEL | |
| | PVAAWRSYLNMELTIPIFATNSDCELIVKAMQGLLKDGNPIPSAI | |
| | AANSGIYSGGSSGGSTLNIEDEYRLHETSKEPDVSLGSTWLSDFP | |
| | QAWAETGGMGLAVRQAPLIIPLKATSTPVSIKQYPMSQEARLGIK | |
| | PHIQRLLDQGILVPCQSPWNTPLLPVKKPGTNDYRPVQDLREVNK | |
| | RVEDIHPTVPNPYNLLSGLPPSHQWYTVLDLKDAFFCLRLHPTSQ | |
| | PLFAFEWRDPEMGISGQLTWTRLPQGFKNSPTLFNEALHRDLADF | |
| | RIQHPDLILLQYVDDLLLAATSELDCQQGTRALLQTLGNLGYRAS | |
| | AKKAQICQKQVKYLGYLLKEGQRWLTEARKETVMGQPTPKTPRQL | |
| | REFLGKAGFCRLFIPGFAEMAAPLYPLTKPGTLFNWGPDQQKAYQ | |
| | EIKQALLTAPALGLPDLTKPFELFVDEKQGYAKGVLTQKLGPWRR | |
| | PVAYLSKKLDPVAAGWPPCLRMVAAIAVLTKDAGKLTMGQPLVIL | |
| | APHAVEALVKQPPDRWLSNARMTHYQALLLDTDRVQFGPVVALNP | |
| | ATLLPLPEEGLQHNCLDILAEAHGTRPDLTDQPLPDADHTWYTDG | |
| | SSLLQEGQRKAGAAVTTETEVIWAKALPAGTSAQRAELIALTQAL | |
| | KMAEGKKLNVYTDSRYAFATAHIHGEIYRRRGWLTSEGKEIKNKD | |
| | EILALLKALFLPKRLSIIHCPGHQKGHSAEARGNRMADQAARKAA | |
| | ITETPDTSTLLIENSSPSGGSKRTADGSEFESPKKKRKVGSGPAA | |
| | KRVKLDGSG | |
| 466(D220R)-MCP- | AATMKRTADGSEFESPKKKRKVSRMSVLTRKVQLIPVGDKEERDR | 1617 |
| MMLV-Brex27--- | VYKYLRDGIEAQNRAMNLYMSGLYFAAINEASKEDRKELNQLYSR | |
| TS long combined | IATSSKGSAYTTDIEFPTGLASTSTLSMAVRQDFTKSLKDGLMYG | |
| | RVSLPTYRKDNPLFVDVRFVALRGTKQKYNGLYHEYKSHTEFLDN | |
| | LYSSDLKVYIKFANDITFQVIFGNPRKSSALRSEFQNIFEEYYKV | |
| | CQSSIQFSGTKIILNMAMRIPDKEIELDEDVCVGVDLGIAIPAVC | |
| | ALNKNRYSRVSIGSKEDFLRVRTKIRNQRKRLQTNLKSSNGGHGR | |
| | KKKMKPMDRFRDYEANWVQNYNHYVSRQVVDFAVKNKAKYINLEN | |
| | LEGIRDDVKNEWLLSNWSYYQLQQYITYKAKTYGIEVRKINPYHT | |
| | SQRCSCCGYEDAGNRPKKEKGQAYFKCLKCGEEMNADFNAARNIA | |
| | MSTEFQSGKKTKKQKKEQHENKKRPAATKKAGQAKKKKSRKRTAD | |
| | GSEFESPKKKRKVASNFTQFVLVDNGGTGDVTVAPSNFANGVAEW | |
| | ISSNSRSQAYKVTCSVRQSSAQKRKYTIKVEVPKVATQTVGGVEL | |
| | PVAAWRSYLNMELTIPIFATNSDCELIVKAMQGLLKDGNPIPSAI | |
| | AANSGIYSGGSSGGSTLNIEDEYRLHETSKEPDVSLGSTWLSDFP | |
| | QAWAETGGMGLAVRQAPLIIPLKATSTPVSIKQYPMSQEARLGIK | |
| | PHIQRLLDQGILVPCQSPWNTPLLPVKKPGTNDYRPVQDLREVNK | |
| | RVEDIHPTVPNPYNLLSGLPPSHQWYTVLDLKDAFFCLRLHPTSQ | |
| | PLFAFEWRDPEMGISGQLTWTRLPQGFKNSPTLFNEALHRDLADF | |
| | RIQHPDLILLQYVDDLLLAATSELDCQQGTRALLQTLGNLGYRAS | |
| | AKKAQICQKQVKYLGYLLKEGQRWLTEARKETVMGQPTPKTPRQL | |
| | REFLGKAGFCRLFIPGFAEMAAPLYPLTKPGTLFNWGPDQQKAYQ | |
| | EIKQALLTAPALGLPDLTKPFELFVDEKQGYAKGVLTQKLGPWRR | |
| | PVAYLSKKLDPVAAGWPPCLRMVAAIAVLTKDAGKLTMGQPLVIL | |
| | APHAVEALVKQPPDRWLSNARMTHYQALLLDTDRVQFGPVVALNP | |
| | ATLLPLPEEGLQHNCLDILAEAHGTRPDLTDQPLPDADHTWYTDG | |
| | SSLLQEGQRKAGAAVTTETEVIWAKALPAGTSAQRAELIALTQAL | |
| | KMAEGKKLNVYTDSRYAFATAHIHGEIYRRRGWLTSEGKEIKNKD | |
| | EILALLKALFLPKRLSIIHCPGHQKGHSAEARGNRMADQAARKAA | |
| | ITETPDTSTLLIENSSPSGGSKRTADGSEFESSATPGSALDFLSR | |
| | LPLPPPVSPICTFVSPAAQKAFQPPRSCGPKKKRKVGSGPAAKRV | |
| | KLDGSG | |
| 466(D220R)-P2A- | AATMKRTADGSEFESPKKKRKVSRMSVLTRKVQLIPVGDKEERDR | 1618 |
| MCP-MMLV---TS | VYKYLRDGIEAQNRAMNLYMSGLYFAAINEASKEDRKELNQLYSR | |
| long combined | IATSSKGSAYTTDIEFPTGLASTSTLSMAVRQDFTKSLKDGLMYG | |
| | RVSLPTYRKDNPLFVDVRFVALRGTKQKYNGLYHEYKSHTEFLDN | |
| | LYSSDLKVYIKFANDITFQVIFGNPRKSSALRSEFQNIFEEYYKV | |
| | CQSSIQFSGTKIILNMAMRIPDKEIELDEDVCVGVDLGIAIPAVC | |
| | ALNKNRYSRVSIGSKEDFLRVRTKIRNQRKRLQTNLKSSNGGHGR | |
| | KKKMKPMDRFRDYEANWVQNYNHYVSRQVVDFAVKNKAKYINLEN | |
| | LEGIRDDVKNEWLLSNWSYYQLQQYITYKAKTYGIEVRKINPYHT | |
| | SQRCSCCGYEDAGNRPKKEKGQAYFKCLKCGEEMNADFNAARNIA | |
| | MSTEFQSGKKTKKQKKEQHENKKRPAATKKAGQAKKKKSRATNFS | |
| | LLKQAGDVEENPGPKRTADGSEFESPKKKRKVASNFTQFVLVDNG | |
| | GTGDVTVAPSNFANGVAEWISSNSRSQAYKVTCSVRQSSAQKRKY | |
| | TIKVEVPKVATQTVGGVELPVAAWRSYLNMELTIPIFATNSDCEL | |
| | IVKAMQGLLKDGNPIPSAIAANSGIYSGGSSGGSTLNIEDEYRLH | |
| | ETSKEPDVSLGSTWLSDFPQAWAETGGMGLAVRQAPLIIPLKATS | |
| | TPVSIKQYPMSQEARLGIKPHIQRLLDQGILVPCQSPWNTPLLPV | |
| | KKPGTNDYRPVQDLREVNKRVEDIHPTVPNPYNLLSGLPPSHQWY | |
| | TVLDLKDAFFCLRLHPTSQPLFAFEWRDPEMGISGQLTWTRLPQG | |
| | FKNSPTLFNEALHRDLADFRIQHPDLILLQYVDDLLLAATSELDC | |
| | QQGTRALLQTLGNLGYRASAKKAQICQKQVKYLGYLLKEGQRWLT | |
| | EARKETVMGQPTPKTPRQLREFLGKAGFCRLFIPGFAEMAAPLYP | |
| | LTKPGTLFNWGPDQQKAYQEIKQALLTAPALGLPDLTKPFELFVD | |
| | EKQGYAKGVLTQKLGPWRRPVAYLSKKLDPVAAGWPPCLRMVAAI | |
| | AVLTKDAGKLTMGQPLVILAPHAVEALVKQPPDRWLSNARMTHYQ | |
| | ALLLDTDRVQFGPVVALNPATLLPLPEEGLQHNCLDILAEAHGTR | |
| | PDLTDQPLPDADHTWYTDGSSLLQEGQRKAGAAVTTETEVIWAKA | |
| | LPAGTSAQRAELIALTQALKMAEGKKLNVYTDSRYAFATAHIHGE | |
| | IYRRRGWLTSEGKEIKNKDEILALLKALFLPKRLSIIHCPGHQKG | |
| | HSAEARGNRMADQAARKAAITETPDTSTLLIENSSPSGGSKRTAD | |
| | GSEFESPKKKRKVGSGPAAKRVKLDGSG | |
| 466(D220R)-P2A- | AATMKRTADGSEFESPKKKRKVSRMSVLTRKVQLIPVGDKEERDR | 1619 |
| MCP-MMLV- | VYKYLRDGIEAQNRAMNLYMSGLYFAAINEASKEDRKELNQLYSR | |
| Brex27---TS long | IATSSKGSAYTTDIEFPTGLASTSTLSMAVRQDFTKSLKDGLMYG | |
| combined | RVSLPTYRKDNPLFVDVRFVALRGTKQKYNGLYHEYKSHTEFLDN | |
| | LYSSDLKVYIKFANDITFQVIFGNPRKSSALRSEFQNIFEEYYKV | |
| | CQSSIQFSGTKIILNMAMRIPDKEIELDEDVCVGVDLGIAIPAVC | |
| | ALNKNRYSRVSIGSKEDFLRVRTKIRNQRKRLQTNLKSSNGGHGR | |
| | KKKMKPMDRFRDYEANWVQNYNHYVSRQVVDFAVKNKAKYINLEN | |
| | LEGIRDDVKNEWLLSNWSYYQLQQYITYKAKTYGIEVRKINPYHT | |
| | SQRCSCCGYEDAGNRPKKEKGQAYFKCLKCGEEMNADFNAARNIA | |
| | MSTEFQSGKKTKKQKKEQHENKKRPAATKKAGQAKKKKSRKRTAD | |
| | GSEFESPKKKRKVASNFTQFVLVDNGGTGDVTVAPSNFANGVAEW | |
| | ISSNSRSQAYKVTCSVRQSSAQKRKYTIKVEVPKVATQTVGGVEL | |
| | PVAAWRSYLNMELTIPIFATNSDCELIVKAMQGLLKDGNPIPSAI | |
| | AANSGIYSGGSSGGSTLNIEDEYRLHETSKEPDVSLGSTWLSDFP | |
| | QAWAETGGMGLAVRQAPLIIPLKATSTPVSIKQYPMSQEARLGIK | |
| | PHIQRLLDQGILVPCQSPWNTPLLPVKKPGTNDYRPVQDLREVNK | |
| | RVEDIHPTVPNPYNLLSGLPPSHQWYTVLDLKDAFFCLRLHPTSQ | |
| | PLFAFEWRDPEMGISGQLTWTRLPQGFKNSPTLFNEALHRDLADF | |
| | RIQHPDLILLQYVDDLLLAATSELDCQQGTRALLQTLGNLGYRAS | |
| | AKKAQICQKQVKYLGYLLKEGQRWLTEARKETVMGQPTPKTPRQL | |
| | REFLGKAGFCRLFIPGFAEMAAPLYPLTKPGTLFNWGPDQQKAYQ | |
| | EIKQALLTAPALGLPDLTKPFELFVDEKQGYAKGVLTQKLGPWRR | |
| | PVAYLSKKLDPVAAGWPPCLRMVAAIAVLTKDAGKLTMGQPLVIL | |
| | APHAVEALVKQPPDRWLSNARMTHYQALLLDTDRVQFGPVVALNP | |
| | ATLLPLPEEGLQHNCLDILAEAHGTRPDLTDQPLPDADHTWYTDG | |
| | SSLLQEGQRKAGAAVTTETEVIWAKALPAGTSAQRAELIALTQAL | |
| | KMAEGKKLNVYTDSRYAFATAHIHGEIYRRRGWLTSEGKEIKNKD | |
| | EILALLKALFLPKRLSIIHCPGHQKGHSAEARGNRMADQAARKAA | |
| | ITETPDTSTLLIENSSPSGGSKRTADGSEFESSATPGSALDFLSR | |
| | LPLPPPVSPICTFVSPAAQKAFQPPRSCGPKKKRKVGSGPAAKRV | |
| | KLDGSG | |

*TS long combined refers to a system in which the retRNA is not circularized.

TABLE 19
Description of gRNAs and retRNAs

| RNA | | SEQ |
|---|---|---|
| Description | Nucleotide Sequence | ID NO |
| gRNA1 | ACAGCUUAUUUGGAAGCUGAAAUGUGAGGUUUAUAACACUCAC | 1620 |
| | AAGAAUCCUGAAAAAGGAUGCCAAACUUCCAAAUCCUGCAUUG | |
| | UUG | |
| gRNA2 | ACAGCUUAUUUGGAAGCUGAAAUGUGAGGUUUAUAACACUCAC | 1621 |
| | AAGAAUCCUGAAAAAGGAUGCCAAACGAAGAACUCAUUACCGC | |
| | UGC | |
| retRNA3 | UGCUCGCUUCGGCAGCACAUAUACUAGUCGACGGGCCGCACUC | 1622 |
| | GCCGGUCCCAAGCCCGGAUAAAAUGGGAGGGGGCGGGAAACCG | |
| | CCUAACCAUGCCGAGUGCGGCCGCAACUAAGCACAUGAGGAU | |
| | CACCCAUGUGCUAAGGGAUAUUUGUUCUUACAGGCAACAAUG | |
| | CAUUACCGCUGCCCAAAAUUUGAAAAACAAGACCAGCAAUCA | |
| | AAUUAACAGUGGCCGCGGUCGGCGUGGACUGUAGAACACUGCC | |
| | AAUGCCGGUCCCAAGCCCGGAUAAAAGUGGAGGGUACAGUCCA | |
| | CGCUCUAGAGCGGACUUCGGUCCGC | |
| retRNA4 | UGCUCGCUUCGGCAGCACAUAUACUAGUCGACGGGCCGCACUC | 1623 |
| | GCCGGUCCCAAGCCCGGAUAAAAUGGGAGGGGGCGGGAAACCG | |
| | CCUAACCAUGCCGAGUGCGGCCGCAACUAAGCACAUGAGGAU | |
| | CACCCAUGUGCGAUUGCUGGUCUUGUUUUUCAAAUUUUGGGC | |
| | AGCGGUAAUGCAUUGUUGCCUGUAAGAACAAAUAUCCCUUAA | |
| | AAUUAACAGUGGCCGCGGUCGGCGUGGACUGUAGAACACUGCC | |
| | AAUGCCGGUCCCAAGCCCGGAUAAAAGUGGAGGGUACAGUCCA | |
| | CGCUCUAGAGCGGACUUCGGUCCGC | |

In rows for gRNA1 and gRNA2, italic sequences represent the spacer sequence targeting DMD; in rows for retRNA3 and retRNA4, bolded sequences represent the template sequence and regular font sequences represent MS2 aptamer and ribozyme sequences.

TABLE 20
Precise Editing Achieved with Dual-Cut, Dual-Flap Systems

| | | % precise |
|---|---|---|
| | % precise | edits with |
| Fusion Protein Description | edits | NHEJi |
| Brex27-466(D220R)-MCP-MMLV---TS long combined* | 1.1 | 0.7 |
| Brex27-466(D220R)-P2A-MCP-MMLV---TS long combined | | 3.1 |
| Brex27-xten20-466(D220R)-MCP-MMLV---TS long combined | 0.8 | 2.7 |
| Brex27-xten20-466(D220R)-P2A-MCP-MMLV---TS long combined | 3.4 | 9.4 |
| 466(D220R)-Brex27-MCP-MMLV---TS long combined | | 3.4 |
| 466(D220R)-Brex27-P2A-MCP-MMLV---TS long combined | 2.0 | 5.3 |
| 466(D220R)-MCP-MMLV---TS long combined | 0.3 | 1.8 |
| 466(D220R)-MCP-MMLV-Brex27---TS long combined | 0.9 | 2.5 |
| 466(D220R)-P2A-MCP-MMLV---TS long combined | 2.2 | 3.0 |
| 466(D220R)-P2A-MCP-MMLV-Brex27---TS long combined | | 11.3 |

*TS long combined refers to a system in which the retRNA is not circularized.

### Example 11. CasM.265466 Nuclease Target Strand (TS) Precision Editing of DMD

This experiment demonstrates precision editing using a fully active nuclease (CasM.265466) that creates double stranded breaks. See FIG. 12 for a non-limiting exemplary illustration of such a system. In this instance, the strand being extended by the RT is the target strand. Precision editing with a nuclease and TS extension is different from standard RT editing done with a non-target strand nickase and non-target strand extension. The target strand extension with Type V Cas proteins, such as CasM.265466, allows one to edit the seed region or PAM of the target, preventing subsequent rounds of targeting, thereby reducing the amount of imprecise indels. The fusion proteins tested are described in TABLE 18 with their corresponding sequences described in Example 10. gRNA1, gRNA2, retRNA3 and retRNA4 are also described in Example 10. The sequence of retRNA1 is UGCUCGCUUCGGCAGCACAUAUACUAGUCGACGGGCCGCACUCGCCGGUCCCAAGCCCGG AUAAAAUGGGAGGGGGCGGGAAACCGCCUAACCAUGCCGAGUGCGGCCGCAACUAAGC ACAUGAGGAUCACCCAUGUGCACAGAGGCGUCCCCAGUACGAAGAACUCAUUACCGG UACCCUGCCCAAAAUUUGAAAAACAAGACCAGCAAUCAAGAGGCUAGAACAAUCAUUA CGGAUCGAAAAUUAACAGUGGCCGCGGUCGGCGUGGACUGUAGAACACUGCCAAUGCCG GUCCCAAGCCCGGAUAAAAGUGGAGGGUACAGUCCACGCUCUAGAGCGGACUUCGGUCCG C (SEQ ID NO: 1624), with the bolded sequence being the template sequence. Briefly, HEK293T cells were transfected with plasmids encoding the various proteins, guide RNAs, and retRNAs. Precise edits were quantified using amplicon NGS. Results are presented in TABLE 21.

TABLE 21
Editing results

| | precise editing | precise editing |
|---|---|---|
| | percent without | percent with |
| Fusion Proteins and RNAs Tested | UBVG08 | UBVG08 |
| Brex27-Nano-MCP-MMLV + TS circular*; gRNA1, retRNA1 | 0.36 | 0.71 |
| Brex27-Nano-P2A-MCP-MMLV + TS circular; gRNA1, | 1.81 | 3.00 |
| retRNA1 | | |
| Nano-MCP-MMLV + TS circular; gRNA1, retRNA1 | 0.21 | |
| Brex27-xten20-Nano-P2A-MCP-MMLV-TS long** 2; gRNA2, | 0.23 | |
| retRNA4 | | |
| Nano-Brex27-MCP-MMLV-TS long 1; gRNA1, retRNA3 | 0.68 | 1.04 |
| Nano-MCP-MMLV-TS long 1; gRNA1, retRNA3 | 0.37 | 0.51 |
| Nano-P2A-MCP-MMLV-TS long 1; gRNA1, retRNA3 | 2.58 | 3.92 |
| Nano-P2A-MCP-MMLV-Brex27-TS long 1; gRNA1, retRNA3 | 3.19 | 11.96 |
| Brex27-Nano-MCP-MMLV-TS long 1; gRNA1, retRNA3 | | 0.39 |
| Brex27-Nano-P2A-MCP-MMLV-TS long 1; gRNA1, retRNA3 | | 1.21 |
| Brex27-xten20-Nano-MCP-MMLV-TS long 1; gRNA1, | | 1.04 |
| retRNA3 | | |
| Brex27-xten20-Nano-P2A-MCP-MMLV-TS long 1; gRNA1, | | 6.16 |
| retRNA3 | | |
| Nano-Brex27-P2A-MCP-MMLV-TS long 1; gRNA1, retRNA3 | | 2.33 |
| Nano-MCP-MMLV-Brex27-TS long 1; gRNA1, retRNA3 | | 2.48 |

*TS circular refers to a system in which the retRNA is circularized (the 5 end is covalently connected to the 3' end.
** TS long refers to a system in which the retRNA is not circularized.

1. A system comprising:
a) an effector protein or a nucleic acid encoding the effector protein;
b) an RNA-directed DNA polymerase (RDDP) or a nucleic acid encoding the RDDP;
c) a guide RNA or a nucleic acid encoding the guide RNA, wherein the guide RNA comprises
i. a first region comprising a protein binding sequence, and
ii. a second region comprising a spacer sequence that hybridizes to a target sequence of a first strand of a double stranded DNA (dsDNA) target nucleic acid, wherein the dsDNA target nucleic acid comprises a human dystrophin gene (DMD),
wherein the first region is located 5′ of the second region; and
d) a template RNA (retRNA) or a nucleic acid encoding the retRNA, wherein the retRNA comprises
i. a primer binding sequence (PBS), and
ii. a template sequence (RTT) that hybridizes to at least a portion of the target sequence of a second strand of the dsDNA target nucleic acid.

2. The system of claim 1, wherein the guide RNA and the retRNA are not linked.

3. The system of claim 1 or 2, wherein the template sequence is located 5′ of the PBS, optionally wherein the 3′ end of the PBS is linked to the 5′ end of the protein binding sequence.

4. The system of any one of claims 1-3, wherein the retRNA is circularized.

5. The system of any one of claims 1-4, wherein the retRNA comprises a protein localization sequence that can localize a protein to the retRNA.

6. The system of claim 5, wherein the protein localization sequence comprises an MS2 coat protein localization sequence.

7. The system of claim 6, wherein the RDDP is not linked to the effector protein, optionally wherein the RDDP is linked to an MS2 coat protein.

8. The system of any one of claims 1-4, wherein the RDDP is linked to the effector protein.

9. The system of any one of claims 1-8, wherein the template sequence comprises a difference of at least one nucleotide relative to an equal length portion of the target sequence.

10. The system of any one of claims 1-9, wherein the effector protein is a Type V Cas protein.

11. The system of any one of claims 1-10, wherein the length of the effector protein is 400 to 800 linked amino acids.

12. The system of any one of claims 1-11, wherein the effector protein comprises an amino acid sequence that is at least 65%, at least 70%, at least 75%, at least 80%, at least 85%, at least 90%, at least 95%, at least 97%, at least 98%, at least 99%, or 100% identical to a sequence in TABLE 1.

13. The system of any one of claims 1-12, wherein the effector protein comprises at least one amino acid alteration relative to a relative amino acid sequence in TABLE 1 that results in reduced nuclease activity, increased nickase activity, or a combination thereof.

14. The system of claim 13, wherein the target nucleic acid comprises a PAM sequence and at least a portion of the PBS is complementary at least a portion of the target nucleic acid sequence that is 5′ of the nucleotide at position 13 relative to the PAM sequence.

15. The system of any one of claims 1-13, wherein the effector protein is a nickase, or wherein the effector protein has nicking activity.

16. The system of any one of claims 1-13, wherein the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 1.

17. The system of claim 16, wherein the effector protein comprises at least one amino acid alteration relative to the amino acid sequence of SEQ ID NO: 1.

18. The system of claim 17, wherein the at least one amino acid alteration comprises an amino acid alteration set forth in TABLE 1.1.

19. The system of claim 18, wherein the at least one amino acid alteration is selected from D220R and A306K, and D220R and K250N.

20. The system of any one of claims 16-19, wherein the guide RNA comprises a nucleotide sequence that is at least 90%, at least 95%, at least 97%, at least 98%, at least 99%, or 100% identical to any one of the nucleotide sequences of SEQ ID NOs: 466-648.

21. The system of claim 20, wherein the spacer sequence comprises a nucleotide sequence that is at least 90%, at least 95%, at least 97%, at least 98%, at least 99%, or 100% identical to any one of the nucleotide sequences of SEQ ID NOs: 100-282.

22. The system of claim 20 or 21, wherein the protein binding sequence comprises a nucleotide sequence that is at least 90%, at least 95%, at least 97%, at least 98%, at least 99%, or 100% identical to the nucleotide sequences of SEQ ID NO: 283.

23. The system of claim 20, wherein the guide RNA comprises a nucleotide sequence that is at least 90%, at least 95%, at least 97%, at least 98%, at least 99%, or 100% identical to any one of the nucleotide sequences of SEQ ID NOs: 649-831.

24. The system of claims 16-23, wherein the retRNA comprises a nucleotide sequence that is at least 90%, at least 95%, at least 97%, at least 98%, at least 99%, or 100% identical to any one of the nucleotide sequences of SEQ ID NOs: 880-903.

25. The system of claim 24, wherein the PBS comprises a nucleotide sequence that is at least 90%, at least 95%, at least 97%, at least 98%, at least 99%, or 100% identical to any one of the nucleotide sequences of SEQ ID NOs: 832-855.

26. The system of claim 24 or 25, wherein the RTT comprises a nucleotide sequence that is at least 90%, at least 95%, at least 97%, at least 98%, at least 99%, or 100% identical to any one of the nucleotide sequences of SEQ ID NOs: 856-879.

27. The system of claim 24, wherein the retRNA comprises a nucleotide sequence that is at least 90%, at least 95%, at least 97%, at least 98%, at least 99%, or 100% identical to any one of the nucleotide sequences of SEQ ID NOs: 904-927.

28. The system of any one of claims 1-15, wherein the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 2.

29. The system of any one of claim 28, wherein the effector protein comprises at least one amino acid alteration relative to the amino acid sequence of SEQ ID NO: 2.

30. The system of claim 29, wherein the at least one amino acid alteration comprises an alteration set forth in TABLE 1.2.

31. The system of claim 30, wherein the at least one amino acid alteration is selected from N355R, N148R, H208R, and a combination thereof.

32. The system of claim 30, wherein the at least one amino acid alteration is selected from L26X and A121Q, K99R and L149R, L26X and N148R, L26X and H208R, N30R and N148R, L26X and N355R, L26X and K99R, L26X and K348R, L26X and A121Q, K99R and N148R, L149R and H208R, L26X and L149R, and S362R and L26X, wherein X is selected from R or K.

33. The system of any one of claims 28-32, wherein the guide RNA comprises a nucleotide sequence that is at least 90%, at least 95%, at least 97%, at least 98%, at least 99%, or 100% identical to any one of the nucleotide sequences of SEQ ID NOs: 1212-1353.

34. The system of claim 33, wherein the spacer sequence comprises a nucleotide sequence that is at least 90%, at least 95%, at least 97%, at least 98%, at least 99%, or 100% identical to any one of the nucleotide sequences of SEQ ID NOs: 928-1069.

35. The system of claim 33 or 34, wherein the protein binding sequence comprises a nucleotide sequence that is at least 90%, at least 95%, at least 97%, at least 98%, at least 99%, or 100% identical to the nucleotide sequences of SEQ ID NO: 6.

36. The system of claim 33, wherein the guide RNA comprises a nucleotide sequence that is at least 90%, at least 95%, at least 97%, at least 98%, at least 99%, or 100% identical to any one of the nucleotide sequences of SEQ ID NOs: 1354-1495.

37. The system of claim 33, wherein the retRNA comprises a nucleotide sequence that is at least 90%, at least 95%, at least 97%, at least 98%, at least 99%, or 100% identical to any one of the nucleotide sequences of SEQ ID NOs: 1544-1567.

38. The system of claim 37, wherein the PBS comprises a nucleotide sequence that is at least 90%, at least 95%, at least 97%, at least 98%, at least 99%, or 100% identical to any one of the nucleotide sequences of SEQ ID NOs: 1496-1519.

39. The system of claim 37 or 38, wherein the RTT comprises a nucleotide sequence that is at least 90%, at least 95%, at least 97%, at least 98%, at least 99%, or 100% identical to any one of the nucleotide sequences of SEQ ID NOs: 1520-1543.

40. The system of claim 37, wherein the retRNA comprises a nucleotide sequence that is at least 90%, at least 95%, at least 97%, at least 98%, at least 99%, or 100% identical to any one of the nucleotide sequences of SEQ ID NOs: 1568-1591.

41. The system of any one of claims 1-40, wherein the primer binding sequence is less than 20, less than 19, less than 18, less than 17, less than 16, less than 16, less than 15, less than 14, less than 13, less than 12, less than 11, less than 10, less than 9, less than 8, less than 7, less than 6, less than 5 nucleotides long, or at least 4 nucleotides long.

42. The system of any one of claims 1-41, wherein the template sequence is less than 35, less than 34, less than 33, less than 32, less than 31, less than 30, less than 29, less than 28, less than 27, less than 26, less than 25, less than 24, less than 23, less than 22, less than 21, less than 20, less than 19, less than 18 nucleotides long, or at least 8 nucleotides long.

43. The system of any one of claims 1-42, wherein the RDDP comprises an amino acid sequence that is at least 65%, at least 70%, at least 75%, at least 80%, at least 85%, at least 90%, at least 95%, at least 97%, at least 98%, at least 99%, or 100% identical to any one of SEQ ID NOs: 11-89.

44. The system of any one of claims 1-43, wherein the RDDP comprises an amino acid sequence that is at least 65%, at least 70%, at least 75%, at least 80%, at least 85%, at least 90%, at least 95%, at least 97%, at least 98%, at least 99%, or 100% identical to any one of SEQ ID NOs: 22, 24, 30, 32, or 40.

45. The system of any one of claims 1-44, comprising an expression vector, wherein the expression vector comprises any combination of: the nucleic acid encoding the effector protein; the nucleic acid encoding the RDDP; the nucleic acid encoding the guide RNA; and the nucleic acid encoding the retRNA.

46. The system of claim 45, wherein the expression vector is an adeno-associated viral (AAV) vector, optionally wherein the AAV vector is an scAAV vector.

47. The system of any one of claims 1-45, comprising a lipid or lipid nanoparticle.

48. The system of any one of claims 1-46, wherein the nucleic acid encoding the effector protein or the nucleic acid encoding the RDDP comprises a messenger RNA.

49. The system of any one of claims 1-47, comprising a non-homologous end joining (NHEJ) inhibitor.

50. The system of any one of claims 1-49, wherein the effector protein comprises four amino acid substitutions relative to SEQ ID NO: 2, wherein the amino acid substitutions comprise L26R, 1471T, S223P, and D703G.

51. The system of claim 50, wherein the effector protein comprises an amino acid sequence that is at least 90%, at least 95%, at least 97%, at least 98%, at least 99%, or 100% identical to SEQ ID NO: 1607.

52. The system of claim 50 or 51, wherein the RDDP is not linked to the effector protein, and wherein the RDDP is linked to an MS2 coat protein (MCP).

53. The system of any one of claims 50-52, wherein the RDDP comprises an amino acid sequence that is at least 90%, at least 95%, at least 97%, at least 98%, at least 99%, or 100% identical to the amino acid sequence of SEQ ID NO: 1608.

54. The system of any one of claims 50-53, wherein the guide RNA comprise a nucleotide sequence that is at least 90%, at least 95%, at least 97%, at least 98%, at least 99%, or 100% identical to the nucleotide sequence of SEQ ID NO: 1327.

55. The system of any one of claims 50-54, wherein the protein binding sequence comprise a nucleotide sequence that is at least 90%, at least 95%, at least 97%, at least 98%, at least 99%, or 100% identical to the nucleotide sequence of SEQ ID NO: 6.

56. The system of any one of claims 50-55, wherein the spacer sequence comprise a nucleotide sequence that is at least 90%, at least 95%, at least 97%, at least 98%, at least 99%, or 100% identical to the nucleotide sequence of SEQ ID NO: 1043.

57. The system of any one of claims 50-56, wherein the retRNA comprises a nucleotide sequence that is at least 90%, at least 95%, at least 97%, at least 98%, at least 99%, or 100% identical to the nucleotide sequence of SEQ ID NO: 1590.

58. The system of any one of claims 50-57, wherein the PBS comprises a nucleotide sequence that is at least 90%, at least 95%, at least 97%, at least 98%, at least 99%, or 100% identical to the nucleotide sequence of SEQ ID NO: 1518.

59. The system of any one of claims 50-58, wherein the RTT comprises a nucleotide sequence that is at least 90%, at least 95%, at least 97%, at least 98%, at least 99%, or 100% identical to the nucleotide sequence of SEQ ID NO: 1542.

60. A pharmaceutical composition comprising the system of any one of claims 1-59; and a pharmaceutically acceptable excipient.

61. A cell modified by the system of any one of claims 1-59.

62. A cell comprising the system of any one of claims 1-59.

63. The cell of claim 61 or 62, wherein the cell is a eukaryotic cell.

64. An expression cassette comprising, from 5′ to 3′:
a) a first inverted terminal repeat (ITR);
b) a first promoter sequence operably linked to a nucleic acid sequence encoding a guide RNA wherein the guide RNA comprises:
i. a first region comprising a protein binding sequence; and
ii. a second region comprising a spacer sequence that is complementary to a target sequence of DMD, wherein the spacer sequence comprises a nucleic acid sequence selected from SEQ ID NOs: 100-282 and 928-1069;
c) a second promoter sequence operably linked to a nucleic acid sequence encoding an effector protein;
d) a poly(A) signal; and
e) a second ITR.

65. The expression cassette of claim 64, wherein the expression cassette further comprises a WPRE sequence located between the nucleic acid sequence encoding an effector protein and the poly(A) signal.

66. The expression cassette of claim 64 or 65, wherein the first promoter is a U6 promoter.

67. The expression cassette of any one of claims 64-66, wherein the second promoter is a CK8E promoter or a SPC5 promoter.

68. The expression cassette of any one of claims 64-67, wherein the poly(A) signal is a bGH or an hGH poly(A) signal.

69. The expression cassette of any one of claims 64-68, wherein the effector protein comprises an amino acid sequence that is at least 90% identical to SEQ ID NO:2.

70. The expression cassette of claim 69, wherein the effector protein comprises the amino acid substitution of L26R relative to SEQ ID NO: 2.

71. The expression cassette of any one of claims 64-68, wherein the effector protein comprises an amino acid sequence that is at least 90% identical to SEQ ID NO:1.

72. The expression cassette of claim 71, wherein the effector protein comprises the amino acid substitution of D220R relative to SEQ ID NO: 1.

73. An adeno-associated virus (AAV) vector comprising the expression cassette of any one of claims 64-72.

74. A method of modifying a target nucleic acid in a cell, the method comprising contacting a target nucleic acid with the system of any one of claims 1-59.

75. The method of claim 74, wherein the cell is a eukaryotic cell.

76. The method of claim 74 or 75, wherein the target nucleic acid is DMD.

77. A method of treating a disease associated with a mutation of a human dystrophin gene in a subject in need thereof, the method comprising administering to the subject:
a) the system of any one of claims 1-59; or
b) the pharmaceutical composition of claim 60; or
c) the AAV vector of claim 73.

78. The method of claim 77, wherein the disease or disorder is any one of the diseases or disorders set forth in TABLE 13.

79. The method of claim 77 or 78, wherein the disease or disorder is Duchenne muscular dystrophy (DMD), becker muscular dystrophy (BMD), or x-linked dilated cardiomyopathy (CMD) Type 3B.

80. The method of any one of claims 77-79, wherein the disease is DMD.

81. A system for deleting a region of DMD, the system comprising:
a) an effector protein or a nucleic acid encoding the effector protein, wherein the effector protein forms a dimer with itself in a cell;
b) an RNA-directed DNA polymerase (RDDP) or a nucleic acid encoding the RDDP;
c) a first guide RNA (gRNA) or a nucleic acid encoding the first gRNA, wherein the first gRNA comprises
i. a first scaffold sequence, and
ii. a first spacer sequence that hybridizes to a first target sequence on a first strand of the DMD gene,
wherein the first scaffold sequence is located 5′ of the first spacer sequence, and
wherein the effector protein and the first gRNA form a first RNP complex that cleaves the DMD gene to form a first single stranded DNA (ssDNA) flap from the first strand of the DMD gene;
d) a second gRNA or a nucleic acid encoding the second gRNA, wherein the second gRNA comprises
i. a second scaffold sequence, and
ii. a second spacer sequence that hybridizes to a second target sequence on a second strand of the DMD gene,
wherein the second scaffold sequence is located 5′ of the second spacer sequence, and
wherein the effector protein and the second gRNA form a second RNP complex that cleaves the DMD gene to form a second ssDNA flap from the second strand of the DMD gene;
e) a first template RNA (retRNA) or a nucleic acid encoding the first retRNA, wherein the first retRNA comprises
i. a first primer binding sequence (PBS) that hybridizes to at least a portion of the first ssDNA flap, and
ii. a first template sequence; and
f) a second retRNA or a nucleic acid encoding the second retRNA, wherein the second retRNA comprises
i. a second PBS that hybridizes to at least a portion of the second ssDNA flap, and
ii. a second template sequence.

82. The system of claim 81, wherein the nucleic acid encoding the effector protein, the RDDP, the gRNAs, and the retRNAs are combined in a single AAV vector.

83. The system of claim 81 or 82, wherein the first spacer sequence and the second spacer sequence hybridize to the first target sequence and the second target sequence of DMD respectively, wherein the first target sequence and the second target sequence are 10 to 10,000 base pairs apart.

84. The system of any preceding claim, comprising a Brex27 peptide or a nucleic acid encoding the Brex27 peptide, optionally wherein the Brex27 peptide comprises an amino acid sequence that is at least 90%, at least 95%, at least 98%, at least 99%, or 100% identical to SEQ ID NO: 1625.

85. A system for modifying a target strand (TS) of a DMD, the system comprising:
a) an effector protein or a nucleic acid encoding the effector protein, wherein the effector protein forms a dimer with itself in a cell;
b) an RNA-directed DNA polymerase (RDDP) or a nucleic acid encoding the RDDP;
c) a guide RNA (gRNA) or a nucleic acid encoding the gRNA, wherein the gRNA comprises
i. a first region comprising a scaffold sequence, and
ii. a second region comprising a spacer sequence that hybridizes to a target sequence of the TS of the DMD gene,
wherein the first region is located 5′ of the second region; and
wherein the effector protein and the gRNA form a RNP complex that produces a double stranded break;
d) a TS template RNA (retRNA) or a nucleic acid encoding the TS retRNA, wherein the TS retRNA comprises
i. a TS primer binding sequence (PBS) that hybridizes to the TS of the DMD gene, and
ii. a TS template sequence.

86. The system of any one of claims 81-85, wherein the effector protein comprises an amino acid sequence that is at least 70%, at least 75%, at least 80%, at least 85%, at least 90%, at least 95%, at least 97%, at least 98%, at least 99%, or 100% identical to SEQ ID NO: 1.

87. The system of claim 86, wherein the effector protein comprises at least one amino acid substitution relative to SEQ ID NO: 1, wherein the amino acid substitution is D220R.

88. The system of any one of claims 81-87, wherein the effector protein is linked to RDDP to form a fusion protein.

89. The system of claim 88, wherein the fusion protein comprises an amino acid sequence that is at least 70%, at least 75%, at least 80%, at least 85%, at least 90%, at least 95%, at least 97%, at least 98%, at least 99%, or 100% identical to an amino acid sequence selected from SEQ ID NOs: 1610-1619.

90. The system of claim 88, wherein the fusion protein comprises an amino acid sequence that is at least 70%, at least 75%, at least 80%, at least 85%, at least 90%, at least 95%, at least 97%, at least 98%, at least 99%, or 100% identical to an amino acid sequence selected from SEQ ID NOs: 1611, 1613, 1615, 1618, and 1619, wherein the amino acid sequence comprises a P2A peptide that results in cleavage of the fusion protein at the site of the P2A.

91. The system of any one of claims 81-90, wherein the gRNA comprises a nucleic acid sequence that is at least 70%, at least 75%, at least 80%, at least 85%, at least 90%, at least 95%, at least 97%, at least 98%, at least 99%, or 100% identical to SEQ ID NO: 1620 or 1621.

92. The system of any one of claims 85-91, wherein the retRNA comprises a nucleic acid sequence that is at least 70%, at least 75%, at least 80%, at least 85%, at least 90%, at least 95%, at least 97%, at least 98%, at least 99%, or 100% identical to a sequence of any one of SEQ ID NO: 1622-1624.

93. The system of claim 85, wherein the nucleic acid sequences encoding the effector protein, the RDDP, the gRNA, and the TS retRNAs are combined in a single AAV vector.

94. The system of claim 93, wherein the nucleic acid sequence encoding the effector protein and the nucleic acid sequence encoding the RDDP is linked by a nucleic acid sequence encoding a P2A peptide.
